(19) United States
(12) Patent Application Publication
(10) Pub. No.: US 2019/0282628 A1
(43) Pub. Date: Sep. 19, 2019

(54) RECOMBINANT BACTERIA ENGINEERED TO TREAT DISEASES AND DISORDERS ASSOCIATED WITH AMINO ACID METABOLISM AND METHODS OF USE THEREOF

(71) Applicant: Synlogic Inc., Cambridge, MA (US)

(72) Inventors: Dean Falb, Sherborn, MA (US); Jonathan W. Kotula, Somerville, MA (US); Vincent M. Isabella, Cambridge, MA (US); Paul F. Miller, Salem, CT (US); Alex Tucker, Somerville, MA (US); Yves Millet, Newton, MA (US); Adam B. Fisher, Cambridge, MA (US)

(21) Appl. No.: 16/069,199

(22) PCT Filed: Jan. 11, 2017
(86) PCT No.: PCT/US2017/013074

§ 371 Date: Jul. 11, 2018

Related U.S. Application Data
(63) Continuation-in-part of application No. 15/379,445, filed on Dec. 14, 2016.
Application No. 15/379,445 is a continuation-in-part of application No. PCT/US2016/069052, filed on Dec. 28, 2016.
Application No. PCT/US2016/069052 is a continuation-in-part of application No. PCT/US2016/062369, filed on Nov. 16, 2016.
Application No. PCT/US2016/062369 is a continuation-in-part of application No. 15/260,319, filed on Sep. 8, 2016.
Application No. 15/260,319 is a continuation-in-part of application No. PCT/US2016/050836, filed on Sep. 8, 2016.
Application No. PCT/US2016/050836 is a continuation-in-part of application No. PCT/US2016/039444, filed on Jun. 24, 2016.
Application No. PCT/US2016/039444 is a continuation-in-part of application No. PCT/US2016/037098, filed on Jun. 10, 2016.
Application No. PCT/US2016/037098 is a continuation-in-part of application No. PCT/US2016/032565, filed on May 13, 2016.
Application No. PCT/US2016/032565 is a continuation-in-part of application No. 15/154,934, filed on May 13, 2016.
Application No. 15/154,934 is a continuation-in-part of application No. PCT/US2016/032562, filed on May 13, 2016.
Application No. PCT/US2016/032562 is a continuation-in-part of application No. PCT/US2016/020530, filed on Mar. 2, 2016.
(60) Provisional application No. 62/439,871, filed on Dec. 28, 2016.
(60) Provisional application No. 62/439,820, filed on Dec. 28, 2016.
(60) Provisional application No. 62/443,639, filed on Jan. 6, 2017.
(60) Provisional application No. 62/434,406, filed on Dec. 14, 2016.
(60) Provisional application No. 62/423,170, filed on Nov. 16, 2016.
(60) Provisional application No. 62/385,235, filed on Sep. 8, 2016.
(60) Provisional application No. 62/362,954, filed on Jul. 15, 2016.
(60) Provisional application No. 62/354,682, filed on Jun. 24, 2016.
(60) Provisional application No. 62/348,360, filed on Jun. 10, 2016.
(60) Provisional application No. 62/348,620, filed on Jun. 10, 2016.
(60) Provisional application No. 62/347,508, filed on Jun. 8, 2016.
(60) Provisional application No. 62/347,576, filed on Jun. 8, 2016.
(60) Provisional application No. 62/345,242, filed on Jun. 3, 2016.
(60) Provisional application No. 62/335,780, filed on May 13, 2016.
(60) Provisional application No. 62/336,338, filed on May 13, 2016.
(60) Provisional application No. 62/277,455, filed on Jan. 11, 2016.
(60) Provisional application No. 62/277,450, filed on Jan. 11, 2016.
(60) Provisional application No. 62/277,413, filed on Jan. 11, 2016.
(60) Provisional application No. 62/314,322, filed on Mar. 28, 2016.
(60) Provisional application No. 62/323,691, filed on Apr. 16, 2016.
(60) Provisional application No. 62/305,462, filed on Mar. 8, 2016.
(60) Provisional application No. 62/297,778, filed on Feb. 19, 2016.
(60) Provisional application No. 62/291,468, filed on Feb. 4, 2016.
(60) Provisional application No. 62/291,461, filed on Feb. 4, 2016.
(60) Provisional application No. 62/291,470, filed on Feb. 4, 2016.
(60) Provisional application No. 62/335,940, filed on May 13, 2016.

(51) Int. Cl. A61K 35/74; C12N 9/88; C12N 9/10; C12N 9/06; C12N 9/04; C12N 9/02; C12N 9/14

(52) U.S. Cl. CPC A61K 35/74; C12Y 401/01028; C12Y 401/01001; C12N 9/1029; C12Y 203/01012; C12N 9/0016; C12Y 104/01009; C12Y 104/01005; C12N 9/0006; C12Y 101/01001; C12N 9/0008; C12Y 102/01003; C12N 9/14; C12Y 307/01003; C12N 9/88

(57) ABSTRACT

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject. These recombinant bacterial cells are designed to drive therapeutic effects throughout the body of a host from a point of origin of the microbiome. Specifically, the present disclosure provides recombinant bacterial cells that comprise an amino acid catabolism enzyme for the treatment of diseases and disorders associated with amino acid metabolism, including cancer, in a subject. The disclosure further provides pharmaceutical compositions and methods of treating disorders associated with amino acid metabolism, such as cancer.

### RELATED APPLICATIONS

This application is a 35 U.S.C. § 371 national stage filing of International Application No. PCT/US2017/013074, filed on Jan. 11, 2017, which in turn claims priority to U.S. Provisional Application No. 62/277,413, filed on Jan. 11, 2016; U.S. Provisional Application No. 62/277,450, filed on Jan. 11, 2016; U.S. Provisional Application No. 62/277,455, filed on Jan. 11, 2016; U.S. Provisional Application No. 62/291,461, filed on Feb. 4, 2016; U.S. Provisional Application No. 62/291,468, filed on Feb. 4, 2016; U.S. Provisional Application No. 62/291,470, filed on Feb. 4, 2016; U.S. Provisional Application No. 62/297,778, filed on Feb. 19, 2016; U.S. Provisional Application No. 62/305,462, filed on Mar. 8, 2016; U.S. Provisional Application No. 62/313,691, filed on Mar. 25, 2016; U.S. Provisional Application No. 62/314,322, filed on Mar. 28, 2016; U.S. Provisional Application No. 62/335,780, filed on May 13, 2016; and U.S. Provisional Application No. 62/335,940, filed on May 13, 2016; U.S. Provisional Application No. 62/336,338, filed on May 13, 2016; U.S. Provisional Application No. 62/345,242, filed on Jun. 3, 2016; U.S. Provisional Application No. 62/347,508, filed on Jun. 8, 2016; U.S. Provisional Application No. 62/347,576, filed on Jun. 8, 2016; U.S. Provisional Application No. 62/348,360, filed on Jun. 10, 2016; U.S. Provisional Application No. 62/348,620, filed on Jun. 10, 2016; U.S. Provisional Application No. 62/354,682, filed on Jun. 24, 2016; U.S. Provisional Application No. 62/362,954, filed on Jul. 15, 2016; U.S. Provisional Application No. 62/385,235, filed on Sep. 8, 2016; U.S. Provisional Application No. 62/423,170, filed on Nov. 16, 2016; U.S. Provisional Application No. 62/434,406, filed on Dec. 14, 2016; U.S. Provisional Application No. 62/439,820, filed on Dec. 28, 2016; U.S. Provisional Application No. 62/439,871, filed on Dec. 28, 2016; and U.S. Provisional Application No. 62/443,639, filed on Jan. 6, 2017; and which is a continuation-in-part of PCT Application No. PCT/US2016/020530, filed on Mar. 2, 2016; a continuation-in-part of PCT Application No. PCT/US2016/032562, filed on May 13, 2016; a continuation-in-part of PCT Application No. PCT/US2016/032565, filed on May 13, 2016; a continuation-in-part of U.S. application Ser. No. 15/154,934, filed on May 13, 2016; a continuation-in-part of PCT Application No. PCT/US2016/037098, filed on Jun. 10, 2016; a continuation-in-part of PCT Application No. PCT/US2016/039444, filed on Jun. 24, 2016; a continuation-in-part of PCT Application No. PCT/US2016/050836, filed on Sep. 8, 2016; a continuation-in-part of U.S. application Ser. No. 15/260,319, filed on Sep. 8, 2016; a continuation-in-part of PCT Application No. PCT/US2016/062369, filed on Nov. 16, 2016; a continuation-in-part of U.S. application Ser. No. 15/379,445, filed on Dec. 14, 2016; a continuation-in-part of PCT Application No. PCT/US2016/069052, filed on Dec. 28, 2016; and a continuation-in-part of PCT Application No. PCT/US2017/013072, filed on Jan. 11, 2017. The entire contents of each of the foregoing which are expressly incorporated herein by reference.

### SEQUENCE LISTING

The instant application contains a Sequence Listing which has been submitted electronically in ASCII format and is hereby incorporated by reference in its entirety. Said ASCII copy, created on Jul. 10, 2018, is named 126046-00704_SL.txt and is 324,166 bytes in size.

### BACKGROUND

It has recently been discovered that the microbiome in mammals plays a large role in health and disease (see Cho and Blaser, Nature Rev. Genet., 13:260-270, 2012 and Owyang and Wu, Gastroenterol., 146(6):1433-1436, 2014). Indeed, bacteria-free animals have abnormal gut epithelial and immune function, suggesting that the microbiome in the gut plays a critical role in the mammalian immune system. Specifically, the gut microbiome has been shown to be involved in diseases, including, for example, diseases associated with amino acid metabolism, cancer, immune diseases (such as Inflammatory Bowel Disease), autism, liver disease, food allergy, metabolic diseases (such as urea cycle disorder, phenylketonuria, and maple syrup urine disease), obesity, and infection, among many others.

With respect to cancers, it is known that many tumors depend on certain amino acids for survival. For example, it is known that melanomas depend on leucine for survival, and that leucine deprivation causes the apoptotic death of melanoma cells through inhibiting mTORC1, the main repressor of autophagy (Shee et al., Cancer Cell, 19:613-628, 2011). Furthermore, it is known that arginine is also necessary for mTORC1 activation. Other groups have demonstrated that deprivation of the amino acids serine and glycine improve survival in cancer based mouse models, and studies have recommended serine-free diets in cancer patients in order to improve their survival odds (Locasale, Nature Reviews, 13:572-583, 2013). Other tumors have been shown to have a dependence on asparagine, such as leukemia (Amylon et al., Leukemia, 13:335-342, 1999).

Moreover, therapeutic administration of isolated recombinant bacterial proteins which catabolize amino acids, such as asparagine, have been approved by the FDA and shown to be therapeutically beneficial and to increase survival for cancer patients (Amylon et al., Leukemia, 13:335-342, 1999). However, patients treated with the isolated amino acid catabolism enzymes have also been shown to develop severe side effects, including an immune response (hypersensitivity) against the asparaginase enzyme (Vrooman et al., Pediatr. Blood Cancer, 54(2):199-205, 2010 and Wetzler, Blood, 124(8): 1206-1207, 2014), and other severe side effects such as coagulopathy, bone marrow suppression, and stroke (Muller, Critical Reviews in Oncology/Hematology, 28(2):97-11, 1998). Accordingly, a need remains for the development of more effective therapeutic options with fewer side effects for treating diseases associated with amino acid metabolism, such as cancer.

### SUMMARY

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject and are designed to drive therapeutic effects throughout the body of a host from a point of origin of the microbiome.

Specifically, the present disclosure provides recombinant bacterial cells, pharmaceutical compositions thereof, and methods of modulating and treating diseases associated with amino acid metabolism, such as cancer and other diseases, such as metabolic diseases and other diseases. Specifically, the recombinant bacteria disclosed herein have been constructed to comprise genetic circuits comprising gene sequence encoding one or more amino acid catabolism enzyme(s). In other embodiments, the recombinant bacteria disclosed herein have been constructed to comprise genetic circuits gene sequence encoding one or more amino acid biosynthetic enzyme(s). In some embodiment, the bacterial cells further comprise other genetic circuitry in order to guarantee the safety and non-colonization of the subject that is administered the recombinant bacteria, such as auxotrophies, kill switches, etc. These recombinant bacteria are safe and well tolerated and augment the innate activities of the subject's microbiome to achieve a therapeutic effect.

In some embodiments, a bacterial cell disclosed herein has been genetically engineered to comprise a heterologous gene sequence encodingone or more amino acid catabolism enzyme(s) and is capable of processing (e.g., metabolizing) and reducing levels of one or more amino acids. In some embodiments, a bacterial cell disclosed herein has been genetically engineered to comprise a heterologous gene sequence encoding one or more amino acid catabolism enzyme(s) and is capable of processing (e.g., metabolizing) and reducing levels of one or more amino acids in low-oxygen environments, e.g., the gut. In some embodiments, a bacterial cell disclosed herein has been genetically engineered to comprise a heterologous gene sequence encoding one or more amino acid biosynthetic enzyme(s) and is capable of producing one or more amino acids, e.g., arginine, thereby increasing levels of one or more amino acids. In some embodiments, a bacterial cell disclosed herein has been genetically engineered to comprise a heterologous gene sequence encoding one or more amino acid biosynthetic enzyme(s) and is capable of producing one or more amino acids, e.g., arginine, thereby increasing levels of one or more amino acids in low oxygen environments, e.g., the gut. Thus, the genetically engineered bacterial cells and pharmaceutical compositions comprising the bacterial cells disclosed herein may be used to convert excess amino acids into non-toxic molecules in order to treat and/or prevent diseases associated with amino acid metabolism, such as cancer. The genetically engineered bacterial cells and pharmaceutical compositions comprising the bacterial cells disclosed herein may be used to produce amino acids, such as arginine, in order to treat and/or prevent diseases associated with amino acid metabolism, such as cancer.

In another embodiment, the amino acid is arginine, lysine, asparagine, serine, glutamine, tryptophan, phenylalanine, leucine, valine, isoleucine, methionine, threonine, cysteine, tyrosine, glutamic acid, histidine, or proline. In one embodiment, the amino acid is not leucine, isoleucine, valine, tryptophan, arginine, or phenylalanine. In another embodiment, the amino acid is not glycine, aspartic acid, or alanine.

In one embodiment, the amino acid is leucine. In another embodiment, the amino acid is isoleucine. In another embodiment, the amino acid is valine. In another embodiment, the amino acid is arginine. In another embodiment, the amino acid is lysine. In another embodiment, the amino acid is asparagine. In another embodiment, the amino acid is serine. In another embodiment, the amino acid is glycine. In another embodiment, the amino acid is glutamine. In another embodiment, the amino acid is tryptophan. In another embodiment, the amino acid is methionine. In another embodiment, the amino acid is threonine. In another embodiment, the amino acid is cysteine. In another embodiment, the amino acid is tyrosine. In another embodiment, the amino acid is phenylalanine. In another embodiment, the amino acid is glutamic acid. In another embodiment, the amino acid is aspartic acid. In another embodiment, the amino acid is alanine. In another embodiment, the amino acid is histidine. In another embodiment, the amino acid is proline.

In one embodiment, the amino acid is not leucine. In another embodiment, the amino acid is not isoleucine. In another embodiment, the amino acid is not valine. In another embodiment, the amino acid is not arginine. In another embodiment, the amino acid is not lysine. In another embodiment, the amino acid is not asparagine. In another embodiment, the amino acid is not serine. In another embodiment, the amino acid is not glycine. In another embodiment, the amino acid is not glutamine. In another embodiment, the amino acid is not tryptophan. In another embodiment, the amino acid is not methionine. In another embodiment, the amino acid is not threonine. In another embodiment, the amino acid is not cysteine. In another embodiment, the amino acid is not tyrosine. In another embodiment, the amino acid is not phenylalanine. In another embodiment, the amino acid is not glutamic acid. In another embodiment, the amino acid is not aspartic acid. In another embodiment, the amino acid is not alanine. In another embodiment, the amino acid is not histidine. In another embodiment, the amino acid is not proline.

### BRIEF DESCRIPTION OF THE DRAWINGS

FIG. 1 depicts the state of one non-limiting embodiment of the amino acid catabolism enzyme construct under inducing conditions. Specifically, FIG. 1 depicts up-regulated amino acid catabolism enzyme production under anaerobic conditions due to FNR dimerizing and inducing FNR responsive promoter-mediated expression of the genes (squiggle) in a recombinant bacterial cell. Each arrow adjacent to one or a cluster of rectangles depicts the promoter responsible for driving transcription, in the direction of the arrow, of such gene(s). Arrows above each rectangle depict the expression product of each gene. The recombinant bacterial cell may further comprise an auxotrophic mutation, and/or a kill switch, as further described herein.

FIG. 2 depicts the generation of a recombinant bacterial strain that has been engineered to catabolize the amino acid, leucine.

FIG. 3 is a graph depicting that recombinant bacteria engineered to catabolize leucine degrade 2 mM leucine in less than 6 hours as compared to a control bacterial strain.

FIG. 4 is a schematic that depicts some of the processes for designing and producing the genetically engineered bacteria of the present disclosure.

FIG. 5 depicts a summary of the benefits of a synthetic biology platform, including the use of metabolic circuit technology and programmable components.

FIG. 6 depicts a model of how recombinant bacteria disclosed herein are designed to be safe, including both inherent safety and engineered safety/waste management using bacterial auxotrophs and kill switches.

FIG. 7 depicts a table of exemplary bacterial genes which may be disrupted or deleted to produce an auxotrophic bacterial cell. These genes include, but are not limited to, genes required for oligonucleotide synthesis, genes required for amino acid synthesis, and genes required for cell wall synthesis.

FIG. 8 depicts a table illustrating the survival of various amino acid auxotrophs in the mouse gut, as detected 24 hours and 48 hours post-gavage. These auxotrophs were generated using BW25113, a non-Nissle strain of E. coli.

FIG. 9 depicts the design of a repression-based kill switch. A repression-based kill switch depends on the presence of an inducer (such as arabinose) to keep the cells alive. The essential gene switch involves the expression of a gene, e.g., DNA polymerase, which is not found in the gut environment. A repression-based kill switch may be toxin-based or essential-gene based.

FIG. 10 depicts the design of a repression-based kill switch. A repression-based kill switch depends on the presence of an inducer (such as arabinose) to keep the cells alive. The essential gene switch involves the expression of a gene, e.g., DNA polymerase, which is not found in the gut environment. A repression-based kill switch may be toxin-based or essential-gene based.

FIG. 11 depicts one example of a repression-based kill switch design. In the presence of Arabinose, the AraC transcription factor is activated and induces expression of TetR. TetR prevents the expression of the toxin. When Arabinose is removed, TetR does not get made and the toxin is produced which eventually overcomes the antitoxin and kills the cell. This circuit is less likely to be inactivated by random mutations than switches where something needs to be produced de novo to kill the cell. The only mutations that inactivate this construct are mutations in the toxin, itself.

FIG. 12 depicts another two examples of a repression-based kill switch design. In circuit 2, in the presence of Arabinose, the AraC transcription factor is activated and induces expression of TetR and the antitoxin. TetR prevents the expression of the toxin. When Arabinose is removed, TetR and the antitoxin do not get made and the toxin is produced which kills the cell. This circuit is less likely to be inactivated by random mutations than switches where something needs to be produced de novo to kill the cell. The only mutations that will inactivate this are mutations in the toxin, itself.

FIGS. 13-17 depict examples of activation-based kill switch designs.

FIG. 13 demonstrates that, in the presence of the inducer molecule, the therapeutic gene, e.g., the amino acid catabolism gene, is expressed, along with a recombinase. The recombinase flips a toxin gene into an active conformation, killing the cell. The natural kinetics of both recombinases provide the timing mechanism for this activation-based kill switch.

FIG. 14 demonstrates that, in the presence of the inducer molecule, the therapeutic gene, e.g., the amino acid catabolism gene, is expressed, along with a recombinase and an anti-toxin. The recombinase flips a toxin gene into an active conformation, and the toxin binds to the anti-toxin being expressed from the inducible promoter. Once the presence of the inducer is lost, the levels of toxin build-up in the cell, overcoming the anti-toxin, and killing the cell.

FIG. 15 is a schematic demonstrating that, when the cell produces equal amounts of toxin and anti-toxin, the cell is stable. However, when the cell no longer produces the anti-toxin, the anti-toxin proteins begin to decay. Once the anti-toxin has decayed completely, the cell dies.

FIG. 16 demonstrates that, in the presence of the inducer molecule, the therapeutic gene, e.g., the amino acid catabolism gene, is expressed, along with a recombinase. The recombinase flips one or more excision enzyme genes (Xis 1 and Xis 2) into an active conformation. The excision enzymes excise one or more essential genes, killing the cell.

FIG. 17 is a schematic depicting an activation-based kill switch, in which Pᵢ is any inducible promoter, e.g., FNR or ROS. When the therapeutic is induced, the anti-toxin and recombinases are immediately turned on, which results in the toxin being ‘flipped’ to the ON position after 4-6 hours, which results in a build-up of anti-toxin before the toxin is expressed. In absence of the inducing signal, only toxin is made and the cell dies.

FIG. 18 depicts an exemplary schematic of the E. coli Nissle 1917 Chromosome.

FIG. 19 depicts a map of integration sites within the E. coli Nissle chromosome. These sites indicate regions where circuit components may be inserted into the chromosome without interfering with essential gene expression. Backslashes (/) are used to show that the insertion will occur between divergently or convergently expressed genes. Insertions within biosynthetic genes, such as thyA, can be useful for creating nutrient auxotrophies. In some embodiments, an individual circuit component is inserted into more than one of the indicated sites.

FIG. 20 depicts three bacterial strains which constitutively express red fluorescent protein (RFP). In strains 1-3, the rfp gene was inserted into different sites in the bacterial chromosome, and resulted in varying degrees of brightness under fluorescent light. Unmodified E. coli Nissle (strain 4) is non-fluorescent.

FIG. 21 depicts a schematic of a wild-type clbA construct and a clbA knock-out construct.

FIG. 22 depicts exemplary sequences of a wild-type clbA construct and a clbA knock-out construct.

FIG. 23 depicts an exemplary manufacturing process for the bacterial cells disclosed herein.

FIG. 24 depicts additional FNR-responsive regulatory regions.

FIG. 25 depicts a graph of Nissle residence in vivo. Streptomycin-resistant Nissle was administered to mice via oral gavage without antibiotic pre-treatment. Fecal pellets from six total mice were monitored post-administration to determine the amount of administered Nissle still residing within the mouse gastrointestinal tract. The bars represent the number of bacteria administered to the mice. The line represents the number of Nissle recovered from the fecal samples each day for 10 consecutive days.

FIG. 26 depicts an example of a genetically engineered bacteria that comprises a plasmid that has been modified to create a host-plasmid mutual dependency, such as the GeneGuard system described in more detail herein.

FIG. 27 depicts the prpR propionate-responsive inducible promoter. The sequence for one propionate-responsive promoter is also disclosed herein as SEQ ID NO:106.

FIG. 28 depicts various branched chain amino acid degradative pathways and the metabolites and associated diseases relating to BCAA metabolism.

FIG. 29 depicts aspects of the branched chain amino acid degradative pathways for leucine, isoleucine, and valine.

FIG. 30 depicts aspects of alternate branched chain amino acid degradative pathways for leucine, isoleucine, and valine involving a ketoacid decarboxylase and an alcohol dehydrogenase, resulting in isopentanol, isobutanol, and 2-methylbutanol, respectively.

FIG. 31 depicts aspects of alternate branched chain amino acid degradative pathways for leucine, isoleucine, and valine involving a ketoacid decarboxylase and an aldehyde dehydrogenase, resulting in isovalerate, isobutyrate, and 2-methylbutyrate, respectively.

FIG. 32. depicts aspects of alternate branched chain amino acid degradative pathways for leucine, isoleucine, and valine involving a branched chain keto acid dehydrogenase complex (bkd), and the Liu operon from Pseudomonas aeruginosa, resulting in the acylCoA derivative of BCAA. In the case of leucine, the Liu operon coverts isovalerylCoA into acetoacetate and acetyl CoA.

FIG. 33 depicts the the conversion of isovaleryCoA to acetoacetate and acetylCoA by the Liu operon enzymes. In the case of isovaleric acidemia, accumulating isovaleric acid can be activated into isoveralyCoA by an acylCoA synthetase, such as LbuL from Steptomyces lividans.

FIG. 34 depicts possible components of a branched chain amino acid synthetic biotic disclosed herein. An exemplary modified bacterium (E. Coli Nissle 1917) for metabolizing leucine to isopentanol may comprise gene sequence(s) for encoding one or more of the following: (1) livKHMGF (a high affinity leucine transporter that can transport leucine into the bacterial cell); (2) LivJHMGF (a high affinity BCAA transporter that can transport leucine, isoleucine, and valine into the bacterial cell); (3) leuDH (leucine dehydrogenase, e.g., derived from P. aeruginosa PAO1 or Bacillus cereus which converts the BCAA into its corresponding α-ketoacid); (4) IlvE (branched chain amino acid aminotransferase, which also converts BCAA into its corresponding α-ketoacid); (5) KivD (branched chain α-ketoacid decarboxylase, e.g., derived from Lactococcus lactis IFPL730, which converts the α-ketoacid to its corresponding aldehyde); and (6) Adh2 (an alcohol dehydrogenase, e.g., derived from S. cerevisiae; which converts the aldehyde to its corresponding alcohol). The bacterium may further be a gene knockout for the gene encoding LeuE (leucine exporter; knocking out this gene keeps intracellular leucine concentration high) and/or the gene encoding IlvC (keto acid reductoisomerase, which is required for BCAA synthesis; knocking out this gene creates an auxotroph and requires the bacterial cell to import isoleucine and valine to survive).

FIG. 35 depicts possible components of a branched chain amino acid synthetic biotic disclosed herein. An exemplary modified bacterium for metabolizing leucine to isopentanol may comprise gene sequence(s) for encoding one or more of the following: (1) livKHMGF (a high affinity leucine transporter that can transport leucine into the bacterial cell); (2) BrnQ (a low affinity BCAA transporter that can transport branched chain amino acids into the bacterial cell); (3) leuDH (leucine dehydrogenase, e.g., derived from P. aeruginosa PAO1 or Bacillus cereus, which converts the BCAA into its corresponding α-ketoacid); (4) IlvE (branched chain amino acid aminotransferase, which also converts BCAA into its corresponding α-ketoacid); (5) L-AAD (amino acid oxidase, which also converts BCAA into its corresponding α-ketoacid; LAAD(Pv)/LAAD(Pm) are from Proteus vulgaris and Proteus mirabilis, respectively); (6) KivD (branched chain α-ketoacid decarboxylase, e.g., derived from Lactococcus lactis IFPL730, which converts the α-ketoacid to its corresponding aldehyde); and (7) an alcohol dehydrogenase (e.g., Adh2, e.g., derived from S. cerevisiae; YghD, e.g., derived from E. coli, which converts the aldehyde to its corresponding alcohol). The bacterium may further be a gene knockout for the gene encoding LeuE (leucine exporter; knocking out this gene keeps intracellular leucine concentration high) and/or the gene encoding IlvC (keto acid reductoisomerase, which is required for BCAA synthesis; knocking out this gene creates an auxotroph and requires the bacterial cell to import isoleucin and valine to survive). An exemplary modified bacterium for metabolizing leucine to isovalerate may comprise gene sequence(s) for encoding one or more of the following: (1) livKHMGF (a high affinity leucine transporter that can transport leucine into the bacterial cell); (2) BrnQ (a low affinity BCAA transporter that can transport branched chain amino acids into the bacterial cell); (3) leudh (leucine dehydrogenase, e.g., derived from P. aeruginosa PAO1 or Bacillus cereus, which converts the BCAA into its corresponding α-ketoacid); (4) IlvE (branched chain amino acid aminotransferase, which also converts BCAA into its corresponding α-ketoacid); (5) L-AAD (amino acid oxidase, which also converts BCAA into its corresponding α-ketoacid); (6) KivD (branched chain α-ketoacid decarboxylase, e.g., derived from Lactococcus lactis IFPL730, which converts the α-ketoacid to its corresponding aldehyde); and (7) an aldehyde dehydrogenase (e.g., PadA, e.g., derived from E. coli K12, which converts the aldehyde to its corresponding carboxylic acid). The bacterium may further be a gene knockout for the gene encoding LeuE (leucine exporter; knocking out this gene keeps intracellular leucine concentration high) and/or the gene encoding IlvC (keto acid reductoisomerase, which is required for BCAA synthesis; knocking out this gene creates an auxotroph and requires the bacterial cell to import isoleucine and valine to survive).

FIG. 36 depicts possible components of a branched chain amino acid synthetic biotic disclosed herein. An exemplary modified bacterium for metabolizing leucine to isopentanol is shown. Leucine is transported into the bacterium via the high affinity leucine transporter, LivKHMGF, where it is converted to alpha-ketoisocaproic acid using leuDH (Leucine dehydrogenase). The alpha-ketoisocaproic acid is converted to isovalderaldehyde using KivD (BCAA α-ketoacid decarboxylase) and further converted to isopentanol using Adh (alcohol dehydrogenase 2). One or more of the catabolic enzymes, transporters, or other genes may be under the control of an inducible promoter that is induced under exogenous environmental conditions, such as any of the inducible promoters provided herein, e.g., a promoter induced under low oxygen or anaerobic conditions.

FIG. 37 depicts one exemplary branched chain amino acid circuit. Genes shown are high affinity leucine transporter complex (LivKHMGF), leucine dehydrogenase (leuDH), e.g., from Pseudomonas aeruginosa, the branched chain α-ketoacid decarboxylase (KivD), e.g., from Lactococcus lactis, and alcohol dehydrogenase 2 (Adh2), e.g., from Saccharomyces cerevisiae. The genes for the leucine exporter (LeuE) and IlvC (keto acid reductoisomerase, required for BCAA synthesis) have been deleted. The gene for LivJ (a BCAA binding protein that can transport branched chain amino acids into the bacterial cell) is added which can be under the control of the native promoter or the constitutive promoter Ptac. One or more of the genes encoding a catabolic enzyme, transporter, and/or other genes (e.g., livJ) may be under the control of an inducible promoter that is induced under exogenous environmental conditions, such as any of the inducible promoters provided herein, e.g., a promoter induced under low oxygen or anaerobic conditions.

FIG. 38 depicts one exemplary branched chain amino acid circuit. Genes shown are high affinity leucine transporter complex (LivKHMGF), BCAA amino transferase (ilvE), the branched chain α-ketoacid decarboxylase (KivD), e.g., from Lactococcus lactis, and alcohol dehydrogenase 2 (Adh2), e.g., from Saccharomyces cerevisiae. The genes for the leucine exporter (LeuE) and keto acid reductoisomerase (IlvC) have been deleted. The gene for LivJ is added which can be under the control of the native promoter or the constitutive promoter Ptac. One or more of the genes encoding a catabolic enzyme, transporter, and/or other genes (e.g., livJ) may be under the control of an inducible promoter that is induced under exogenous environmental conditions, such as any of the inducible promoters provided herein, e.g., a promoter induced under low oxygen or anaerobic conditions.

FIG. 39 A-F depicts exemplary components of branched chain amino acid synthetic biotics. FIG. 39A and FIG. 39A depicts 2 exemplary components of a branched chain amino acid synthetic biotic disclosed herein for leucine catabolism to isopentanol or isovalerate (FIG. 39A) or alpha-ketoisocaproic acid (FIG. 39B), wherein the second step is catalyzed by Ketoacid decarboxylase (KivD). FIG. 39C depicts a schematic of the corresponding metabolic pathway for FIG. 39A and FIG. 39B. In some embodiments, both circuits can be expressed in the same strain. Alternatively, the circuits can each be expressed individually. Genes shown in FIGS. 13A and B are amino transferase (ilvE), leuDH (derived from P. aeruginosa PAO1 or Bacillus cereus) and/or LAAD (derived from Proteus mirabilis or Proteus vulgaris) for conversion of BCAA to the α-keto acid; the branched chain α-ketoacid decarboxylase (KivD) for conversion from the α-keto acid to the corresponding aldehyde; and alcohol dehydrogenase 2 (Adh2; yqhD) for conversion to the corresponding alcohol or aldehyde dehydrogenase (padA) for conversion to the corresponding carboxylic acid. The genes for the leucine exporter (LeuE) and keto acid reductoisomerase (ilvC) can be deleted. FIG. 39D and FIG. 39E depict 2 exemplary components of a branched chain amino acid synthetic biotic disclosed herein for leucine catabolism to isovalerylCoA (FIG. 39E) or alpha-ketoisocaproic acid (FIG. 39E and FIG. 39F), wherein the second step is catalyzed by Bkd complex from Pseudomonas aeruginosa. FIG. 39F depicts a schematic of the corresponding metabolic pathway for FIG. 39D and FIG. 39E. In some embodiments, both circuits can be expressed in the same strain. Alternatively, the circuit shown in FIG. 39D can each be expressed individually, without the circuit of FIG. 39E. The circuit in FIG. 39E (the Liu operon) requires the circuit of FIG. 39D to to generate its substrate, isovalerylCoA, and therefore is used together with the the circuit of FIG. 39E. Genes shown in FIG. 13D and FIG. 13E are amino transferase (ilvE), leuDH (derived from P. aeruginosa PAO1 or Bacillus cereus) and/or LAAD (derived from Proteus mirabilis or Proteus vulgaris) for conversion of BCAA to the α-keto acid; the Bkd complex (comprising bkdA1, bkdA2, bkdB, and lpdV) for conversion from the α-keto acid to the corresponding CoA thioester, and the Liu operon (comprising liuA, liuB, liuC, liuD, and liuE) for conversion of isovaleryl-CoA to acetoacetate and acetylCoA. One or more of the genes encoding a catabolic enzyme, transporter, and/or other genes may be under the control of an inducible promoter that is induced under exogenous environmental conditions, such as any of the inducible promoters provided herein, e.g., a promoter induced under low oxygen or anaerobic conditions. In certain embodiments, the constructs are expressed on a high copy plasmid. In certain embodiments, any of the genes may be under the control of a tetR promoter. For example, the construct may comprise a (constitutive or inducible) promoter driving expression of the Tet repressor (TetR) from the tetR gene, which is linked to a second promoter comprising a TetR binding site that drives expression of any of the BCAA catabolic cassettes described above. TetR is (either constitutively or inducibly) expressed and inhibits the expression of the BCAA catabolic cassette(s). Upon addition of anhydrotetracylcine (ATC), TetR binds to ATC and binds removing the inhibition by TetR allowing expression of the BCAA catabolic cassettes.

FIG. 40 depicts one exemplary branched chain amino acid circuit. Genes shown are high affinity leucine transporter complex (LivKHMGF), L-AAD, e.g., derived from Proteus vulgaris or Proteus mirabilis, the branched chain α-ketoacid decarboxylase (KivD), e.g., from Lactococcus lactis, and either aldehyde dehydrogenase (PadA), e.g., from E. Coli K-12, alcohol dehydrogenase YqhD, e.g., from E. Coli, or alcohol dehydrogenase Adh2 from S. cerevisiae. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 41 depicts one exemplary branched chain amino acid circuit. Genes shown are high affinity leucine transporter complex (LivKHMGF), LeuDh, e.g., derived from Pseudomonas aeruginosa PAO1 or Bacillus cereus, the branched chain α-ketoacid decarboxylase (KivD), e.g., from Lactococcus lactis, and either aldehyde dehydrogenase (PadA), e.g., from E. Coli K-12, alcohol dehydrogenase YqhD from E. Coli, or alcohol dehydrogenase Adh2, e.g., from S. cerevisiae. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 42 depicts one exemplary branched chain amino acid circuit. Genes shown are high affinity leucine transporter complex (LivKHMGF), low affinity BCAA transporter (brnQ), a leucine dehydrogenase leuDH (from Pseudomonas aeruginosa or Bacillus cereus), Bkd complex (comprising bkdA1, bkdA2, bkdB, and lpdV) for conversion from the α-keto acid to the corresponding CoA thioester. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 43 depicts one exemplary branched chain amino acid circuit. Genes shown are high affinity leucine transporter complex (LivKHMGF), low affinity BCAA transporter (brnQ), a leucine dehydrogenase leuDH (from Pseudomonas aeruginosa or Bacillus cereus), the Bkd complex (comprising bkdA1, bkdA2, bkdB, and lpdV) for conversion from the α-keto acid to the corresponding CoA thioester, and Liu operon (comprising liuA, liuB, liuC, liuD, and liuE) for conversion of isovaleryl-CoA to acetoacetate and acetylCoA. The genes for the leucine exporter (LeuE) and IlvC have been deleted. The gene for BrnQ is added. In some embodiments, any of the genes may be under the control of a promoter inducible under low oxygen or anaerobic conditions, e.g., an FNR promoter.

FIG. 44 depicts one exemplary branched chain amino acid circuit.

FIG. 45 depicts exemplary constructs of circuit components for LeuDH, kivD and livKHMGF inducible expression in E. coli. FIG. 45A depicts kivD under the control of the Tet promoter, e.g., cloned in a high-copy plasmid. FIG. 45B depicts kivD and LeuDH under the control of the Tet promoter, e.g., cloned into a high-copy plasmid. FIG. 45C depicts livKHMGF operon under the control of the Tet promoter, flanked by the lacZ homologous region for chromosomal integration by lamba-red recombination.

FIG. 46 depicts the gene organization of the Tet-livKHMGF construct.

FIG. 47 depicts leucine levels in the Nissle ΔleuE deletion strain harboring a high-copy plasmid expressing kivD from the Tet promoter or further with a copy of the livKHMGF operon driven by the Tet promoter integrated into the chromosome at the lacZ locus, which were induced with ATC and incubated in culture medium supplemented with 2 mM leucine. Samples were removed at 0, 1.5, 6 and 18 h, and leucine concentration was determined by liquid chromatography tandem mass spectrometry.

FIG. 48 depicts leucine degradation in the Nissle ΔleuE deletion strain harboring a high-copy plasmid expressing the branch-chain keto-acid dehydrogenase (bkd) complex (comprising bkdA1, bkdA2, bkdB, and lpdV) with or without expression of a leucine dehydrogenase (LeuDH) from the Tet promoter or further with a copy of the leucine importer livKHMGF driven by the Tet promoter integrated into the chromosome at the lacZ locus, which were induced with ATC and incubated in culture medium supplemented with 2 mM leucine. Samples were removed at 0, 1.5, 6 and 18 h, and leucine concentration was determined by liquid chromatography tandem mass spectrometry.

FIGS. 49A, 49B, and 49C depict the simultaneous degradation of leucine (FIG. 49A), isoleucine (FIG. 49B), and valine (FIG. 49C) by E. coli Nissle and its ΔleuE deletion strain harboring a high-copy plasmid expressing the keto-acid decarboxylase kivD from the Tet promoter or further with a copy of the livKHMGF operon driven by the Tet promoter integrated into the chromosome at the lacZ locus, which were induced with ATC and incubated in culture medium supplemented with 2 mM leucine, 2 mM isoleucine and 2 mM valine. Samples were removed at 0, 1.5, 6 and 18 h, and BCAA concentration was determined by liquid chromatography tandem mass spectrometry. The strains were grown overnight at 37° C. in LB media, and the overnight culture was used to inoculate a new batch at a 1/100 dilution in LB, which was grown for three hours at 37° C. Induction was for two hours with 100 ng/mL ATC. The cells were then collected by centrifugation and resuspended in M9+0.5% glucose and 2 mM each of leucine, isoleucine, and valine. Samples were removed at 0, 1.5, 6 and 18 h, and BCAA concentration was determined by liquid chromatography tandem mass spectrometry. The results demonstrate that isoleucine and valine were also consumed by leucine-degrading strains. Moreover, deletion of leuE and expression of livKHMGF improved the rate of BCAA degradation.

FIG. 50 depicts a bar graph showing that the expression of kivD in E. coli Nissle leads to leucine degradation in vitro. The strains were grown overnight at 37° C. in LB media, and the overnight culture was used to inoculate a new batch at a 1/100 dilution in LB. Induction was for two hours with 100 ng/mL ATC. The cells were then collected by centrifugation and resuspended in M9+0.5% glucose and 2 mM leucine. Aliquots were removed at the indicated times for leucine determination by mass spectrometry. Inclusion of kivD resulted in increased bacterial cell consumption of leucine.

FIGS. 51A and 51B depict the determination of the leucine degradation rate, as mediated by KivD. The strains were grown overnight at 37° C. in LB media, and the overnight culture was used to inoculate a new batch at a 1/100 dilution in LB, which was grown for two hours at 37° C. Induction was for one hour with 100 ng/mL ATC. The cells were then collected by centrifugation and resuspended in M9+0.5% glucose and 2 mM leucine at OD₆₀₀=1. Samples were collected at 3 hours. The total degradation rate was about 250 nmol/10⁹ CFU/hour. The degradation rate attributable to KivD was about 50 nmol/10⁹ CFU/hour.

FIGS. 52A, 52B, and 52C depicts bargraphs which shows the efficient degradation of leucine (FIG. 52A), isoleucine (FIG. 52B), and valine (FIG. 52C) by the engineered strains. FIG. 52D depicts a bargraph showing that expression of leucine dehydrogenase (LeuDH from Pseudomonas aeruginosa) improves the rate of leucine degradation to about 160 nmol/10⁹ CFU/hour. The background strain is Nissle ΔleuE, lacZ:tet-livKHMGF.

FIG. 53 depicts the pathway of leucine degradation and KIC degradation engineered into the SYN469 strain.

FIGS. 54A and 54B depicts the rate of leucine degradation or KIC degradation in several different engineered bacteria. The background strain used was SYN469 (ΔleuEΔilvC, lacZ:tet-livKHMGF), and the circuit was under the control of the Tet promoter on a high-copy plasmid. SYN479, SYN467, SYN949, SYN954, and SYN950 strains were fed leucine (FIG. 54A) or ketoisocaproate (KIC, also known as 4-methyl-2-oxopentanoate) (FIG. 54B), and products were monitored. A higher conversion of KIC than leucine to end-products demonstrates that leucine uptake and/or conversion to KIC is rate-limiting.

FIG. 55 depicts the use of valine sensitivity in E. coli as a genetic screening tool. There are three AHAS (acetohydroxybutanoate synthase) isozymes in E. coli (AHAS I: ilvBN, AHAS II: ilvGM, and AHAS III: ilvIH). Valine and leucine exert feedback inhibition on AHAS I and AHAS III; AHAS II is resistant to Val and Leu inhibition. E. coli K12 has a frameshift mutation in ilvG (AHAS II) and is unable to produce isoleucine and leucine in the presence of valine. Nissle has a functional ilvG and is insensitive to valine and leucine. A genetically engineered strain derived from E. coli K12, which more efficiently degrades leucine, has a greater reduction in sensitivity to leucine (through relieving the feedback inhibition on AHAS I and III). As a result, this pathway can be used as a tool to select snf identify a strain with improved resistance to leucine.

FIG. 56A depicts a bar graph showing the the leucine degration rates for various engineered bacterial strains. Bacterial strain Syn 469 is a leuE and ilvC knockout and comprises the leucine transporter under the control of tet promoter. Other tested engineered bacterial strains include: (1) strain having ilvE, kivD, and adh2; (2) strain having leuDh, kivD, and adh2; and (3) strain having L-AAD, kivD, and adh2. The strains are tet-inducible constructs on a high copy plasmid. The results show that L-amino acid deaminase (L-AAD) provides the best leucine degradation rate. FIG. 56B depicts a schematic of the corresponding pathways.

FIG. 57A shows the leucine degration rates for various engineered bacterial strains. Bacterial strain Syn 469 is a leuE and ilvC knockout and comprises the leucine transporter under the control of tet promoter. Other tested engineered bacterial strains include: (1) strain having L-AAD derived from P. vulgaris, kivD, and adh2; (2) strain having L-AAD derived from P. vulgaris (LAAD_{Pv}), kivD, and yqhD; (3) strain having L-AAD derived from P. vulgaris, kivD, and padA and (4) strain having L-AAD derived from P. mirabilis (LAAD_{Pm}). The results show that yqhD, adh2, and padAhave similar activities and that LAAD_{Pm} is a good alternative to LAAD_{Pv}. FIG. 57B depicts a schematic of the corresponding pathways.

FIG. 58A shows the leucine degration rates for various engineered bacterial strains. Bacterial strain Syn 458 is a leuE knockout. Syn 452 is a leuE knockout and comprises the leucine transporter under the control of tet promoter. These background strains were tested with bacterial strains additionally having leuDH derived from P. aeruginosa, kivD, and padA. The results show that overexpression of the high affinity leucine transporter livKHMGF does not dramatically improved the rate of leucine degradation in a LeuE knockout strain having LeuDH, kivD, and padA with and without the leucine transporter livKHMGF under the control of tet promoter as measured by leucine degradation, KIC production, and isovalerate production. FIG. 58B depicts a schematic of the corresponding pathways.

FIGS. 59A and 59B depict a bar graph which shows the leucine degration rates for various engineered bacterial strains. SYN469 is a LeuE and ilvC knockout bacterial strain and comprises the leucine transporter under the control of a tet promoter. The tet inducible leuDH-kivD-padA construct was expressed on a high copy plasmid. Two different leucine dehydrogenases were used in the tested constructs: leuDH_{PA} derived from P. aeruginosa and leuDH_{BC} derived from Bacillus cereus. The tet inducible brnQ construct was expressed on a low copy plasmid. FIG. 59A depicts a bargraph which shows that overexpression of the low-affinity BCAA transporter BrnQ greatly improves the rate of leucine degradation in a LeuE and ilvC knockout bacterial strain having either LeuDH derived from P. aeruginosa or LeuDH derived from Bacillus cereus, kivD, and padA with and without the BCAA transporter brnQ under the control of tet promoter as measured by leucine degradation, KIC production, and isovalerate production. FIG. 59B depicts a bar graph which shows the overexpression of the low-affinity BCAA transporter BrnQ greatly improves the rate of leucine degradation in leuDH-kivD-padA constructs. FIG. 59C depicts a schematic of the corresponding pathways.

FIG. 60 depicts a graph which shows that leucine is able to recirculate from the the periphery into the small intestine. BL6 animals were subjected to subcutaneous injection of isotopic leucine (¹³C₆) (0.1 mg/g). Plasma, small intestine (SI), large intestine (LI) and cecum effluent was tested for the presence of ¹³C₆-Leucine.

FIG. 61 depicts a bar graph showing the efficient import of valine by the expression of an inducible leucine high affinity transporter, livKHMGF, and the constitutive expression of livJ encoding for the BCAA binding protein of the BCAA high affinity transporter livJHMHGF. The natural secretion of valine by E. coli Nissle is observed for the ΔleuE strain. The secretion of valine is strongly reduced for ΔleuE, lacZ:Ptet-livKHMGF in the presence of ATC. This strongly suggests that the secreted valine is efficiently imported back into the cell by livKHMGF. The secretion of valine is abolished in the ΔleuE, lacZ:Ptet-livKHMGF, Ptac-livJ strain, with or without ATC. This strongly suggests that the constitutive expression of livJ is sufficient to import back the entire amount of valine secreted by the cell via the livJHMGF transporter.

FIG. 62A and FIG. 62B depict bar graphs of leucine concentrations (FIG. 62A) and degradation rates (FIG. 62B) measured in an in vitro leucine degradation assay comparing strains with (SYN1980) and without (SYN1992) a tetracycline inducible brnQ construct. FIG. 62A depicts a bar graph of leucine concentrations present at 0, 1.5 and 3 h in the media of SYN1992 (ΔleuE, ΔilvC, lacZ:tetR-Ptet-livKHMGF, tetR-Ptet-leuDH(Bc)-kivD-adh2-rrnB ter (pSC101)) and SYN1980 (ΔleuE, ΔilvC, lacZ:tetR-Ptet-livKHMGF, tetR-Ptet-leuDH(Bc)-kivD-adh2-brnQ-rrnB ter (pSC101)). SYN469 (comprising ΔleuE, ΔilvC, and integrated lacZ:tetR-Ptet-livKHMGF) was used as a control. FIG. 62B depicts a bar graph showing the leucine degradation rates for SYN1992, SYN1980, and SYN469 in the presence and absence of ATC. Leucine degradation rates were increased in both SYN1992 and SYN1980 upon addition of tetracycline, with SYN1980 (comprising tet-inducible BrnqQ) having a greater overall degradation rate. FIG. 62C depicts a schematic of a construct comprising codon optimized LeuDH-kivD-adh2-brnQ construct driven by a tetracycline inducible promoter, e.g., as used in FIG. 62A and FIG. 62B. FIG. 62D depicts a schematic of a construct comprising codon optimized LeuDH-kivD-padA-brnQ construct driven by a tetracycline inducible promoter; in other embodiments, the construct can be driven by a different promoter, e.g., an FNR promoter. FIG. 62E depicts a schematic of a construct comprising codon optimized LeuDH-kivD-yqhD-brnQ construct driven by a tetracycline inducible promoter; in other embodiments, the construct can be driven by a different promoter, e.g., an FNR promoter.

FIG. 63A and FIG. 63B depict bar graphs of leucine concentrations (FIG. 63A) and degradation rates (FIG. 63B) measured in an in vitro leucine degradation assay comparing strains with (SYN1981) and without (SYN1993) a tetracycline inducible brnQ construct. FIG. 63A depicts a bar graph of leucine concentrations present at 0, 1.5 and 3 h in the media of SYN1993 (ΔleuE, ΔilvC, lacZ:tetR-Ptet-livKHMGF, PfnrS-leuDH(Bc)-kivD-adh2-rrnB ter (pSC101)) and SYN1981 (ΔleuE, ΔilvC, lacZ:tetR-Ptet-livKHMGF, PfnrS-leuDH(Bc)-kivD-adh2-brnQ-rrnB ter (pSC101)). SYN469 (comprising ΔleuE, ΔilvC, and integrated lacZ:tetR-Ptet-livKHMGF) was used as a control. FIG. 63B depicts a bar graph showing the leucine degradation rates for SYN1993, SYN1981, and SYN469 with or without anaerobic induction of FNR mediated expression. Leucine degradation rates were increased in both SYN1993 and SYN1981 upon anaerobic induction, with SYN1981 (comprising FNR-inducible BrnqQ) having a greater overall degradation rate. FIG. 63C depicts a schematic of a construct comprising codon optimized LeuDH-kivD-adh2-brnQ construct driven by an FNR promoter, e.g., as used in FIG. 63A and FIG. 63B.

FIG. 64 depicts a synthetic biotic for treating phenylketonuria (PKU) and disorders characterized by hyperphenylalaninemia.

FIG. 65 depicts an exemplary synthetic biotic for treating phenylketonuria (PKU) and disorders characterized by hyperphenylalaninemia.

FIG. 66 depicts an exemplary synthetic biotic for treating phenylketonuria (PKU) and disorders characterized by hyperphenylalaninemia.

FIG. 67A depicts phenylalanine concentrations in samples comprising bacteria expressing PAL1 or on low-copy (LC; SYN-PKU101) or high-copy (HC; SYN-PKU102) plasmids or PAL3 on low-copy (LC; SYN-PKU201) or high-copy (HC; SYN-PKU202) plasmids, induced with anhydrous tetracycline (ATC), and then grown in culture medium supplemented with 4 mM (660,000 ng/mL) of phenylalanine. Samples were removed at 0 hrs, 4 hrs, and 23 hrs. Phenylalanine concentrations were determined by mass spectrometry. FIG. 67B depicts cinnamate levels in samples at 4 hrs and 23 hrs post-induction. In PAL3-expressing strains, the PAL3 gene is derived from Photorhabdus luminescens, an enterobacterium in the same taxonomic subdivision as Escherichia coli.

FIG. 68A depicts phenylalanine concentrations in samples comprising bacteria expressing PAL1 or PAL3 on low-copy (LC) or high-copy (HC) plasmids, or further comprising a copy of pheP driven by the Tet promoter integrated into the chromosome. Bacteria were induced with ATC, and then grown in culture medium supplemented with 4 mM (660,000 ng/mL) of phenylalanine to an OD₆₀₀ of 2.0. Samples were removed at 0 hrs, 2 hrs, and 4 hrs post-induction and phenylalanine concentrations were determined by mass spectrometry. Notably, the additional copy of pheP enhanced the degradation of phenylalanine (4 mM) in 4 hrs. FIG. 68B depicts cinnamate levels in samples at 2 hrs and 4 hrs post-induction. In some embodiments, cinnamate may be used as an alternative biomarker for strain activity. PheP overexpression improves phenylalanine metabolism in engineered bacteria. Strains analyzed in this data set are SYN-PKU101, SYN-PKU102, SYN-PKU202, SYN-PKU201, SYN-PKU401, SYN-PKU402, SYN-PKU203, SYN-PKU302, SYN-PKU303.

FIG. 69 depicts phenylalanine concentrations in cultures of synthetic probiotic strains, with and without an additional copy of pheP inserted on the chromosome. After 1.5 hrs of growth, cultures were placed in Coy anaerobic chamber supplying 90% N₂, 5% CO₂, and 5% H₂. After 4 hrs of induction, bacteria were resuspended in assay buffer containing 4 mM phenylalanine. Aliquots were removed from cell assays every 30 min for 3 hrs for phenylalanine quantification by mass spectrometry. Phenylalanine degradation rates in strains comprising an additional copy of pheP (SYN-PKU304 and SYN-PKU305; left) were higher than strains lacking an additional copy of pheP (SYN-PKU308 and SYN-PKU307; right).

FIG. 70 depicts trans-cinnamate concentrations (PAL activity) for strains comprising single PAL3 insertions at various locations on the chromosome.

FIG. 71 depicts trans-cinnamate concentrations (PAL activity) for strains comprising multiple PAL3 insertions at various locations on the chromosome.

FIG. 72 depicts phenylalanine concentrations in cultures of synthetic probiotic strain SYN-PKU511 over time. After 2.5 hrs of growth, cultures were placed in Coy anaerobic chamber supplying 90% N2, 5% CO2, and 5% H2. After 3.5 hrs of induction in phenylalanine containing medium, whole cell extracts were prepared every 30 min for 3 hrs and phenylalanine was quantified by mass spectrometry. SYN-PKU511 comprises 5 integrated copies of an anaerobically (FNR) controlled gene encoding phenylalanine ammonia lyase (PAL) at 5 chromosomal locations and an anaerobically controlled gene encoding a high affinity Phe transporter (pheP) integrated in the lacZ locus.

FIGS. 73A and 73B depict phenylalanine concentrations in cultures of a synthetic probiotic strain, SYN-PKU401, which comprises a high copy pUC57-plasmid with LAAD driven by a Tet inducible promoter, cells were grown in flasks shaking at 37 C, and induced with TCA at early log phase for a duration of 2 hours. Cells were spun down and re-suspended in assay buffer containing phenylalanine. Cells were measured at various cell concentrations and at varying oxygen levels. Cells were incubated aerobically (1 ml) in a 14 ml culture tube, shaking at 250 rpm, incubated under microaerobic conditions, 1 ml cells incubated in a 1.7 ml conical tube without shaking, or incubated anaerobically in a Coy anaerobic chamber supplying 90% N2, 5% CO2, and 5% H2. Aliquots were removed from cell assays every 30 min for 2 hrs for phenylalanine quantification by mass spectrometry. FIG. 73A depicts phenylalanine concentrations under aerobic conditions using two cell densities. A and B are duplicates under the same experimental conditions. The activity in aerobic conditions is ˜50 umol/hr./1e9 cells. FIG. 73B depicts phenylalanine concentrations of aerobically, microaerobically, or anaerobically grown cells.

FIG. 74A shows phenylalanine concentrations before and after feeding in an in vivo mouse model of PKU. At the beginning of the study, homozygous BTBR-Pah^{enu2} mice were given water supplemented with 100 micrograms/mL ATC and 5% sucrose. Mice were fasted by removing chow overnight (10 hrs), and blood samples were collected by mandibular bleeding the next morning in order to determine baseline phenylalanine levels. Mice were given chow again, gavaged with 100 microliters (5×10⁹ CFU) of bacteria (SYN-PKU302 or control Nissle) after 1 hr., and allowed to feed for another 2 hrs. Serum phenylalanine concentrations were determined 2 hrs post-gavage. FIG. 74B shows the percent (%) change in blood phenylalanine concentrations before and after feeding as a male or female group average (p<0.01).

FIGS. 75A and 75B depict blood phenylalanine concentrations relative to baseline following subcutaneous phenylalanine challenge in an in vivo mouse model of PKU. Mice were orally gavaged with 200 μL of H₂O (n=30), SYN-PKU901 (n=33), or SYN-PKU303 (n=34) at 30 and 90 minutes post-phenylalanine injection (0.1 mg/gram of average group body weight). FIGS. 75A and 75B show blood phenylalanine concentrations at 2 hrs and 4 hrs post-phenylalanine injection, respectively. These data indicate that oral administration of the engineered probiotic strain SYN-PKU303 significantly reduces blood phenylalanine levels in mice, compared to mice administered mock treatment (H₂O) or the parental strain (SYN-PKU901) (*, p<0.05; ***, p<0.001; ****, p<0.00001). SYN-PKU303 is capable of intercepting enterorecirculating phenylalanine.

FIG. 76 depicts blood phenylalanine concentrations relative to baseline following subcutaneous phenylalanine challenge in an in vivo mouse model of PKU. Mice were orally gavaged with 200 μL of H₂O (n=30), SYN-PKU901 (n=33), SYN-PKU303 (n=34), or SYN-PKU304 (n=34) at 30 and 90 minutes post-phenylalanine injection (0.1 mg/gram of average group body weight). Blood phenylalanine concentrations post phenylalanine injection indicate that SYN-PKU304 (low copy plasmid containing fnrS-PAL) is at least as effective as SYN-PKU303 (high copy plasmid containing Tet-PAL) in reducing circulating Phe levels in the enterorecirculation model.

FIGS. 77A-D depict blood phenylalanine concentrations relative to baseline following subcutaneous phenylalanine challenge in an in vivo mouse model of PKU. Mice were orally gavaged with H2O, SYN-PKU901, SYN-PKU303, or SYN-PKU304 at 30 and 90 minutes post-phenylalanine injection (0.1 mg/gram of average group body weight). FIGS. 77A and 77B show blood phenylalanine concentrations at 2 hrs and 4 hrs post-phenylalanine injection, respectively. These data indicate that oral administration of engineered probiotic strains SYN-PKU303 and SYN-PKU304 significantly reduces blood phenylalanine levels in mice compared to mice administered mock treatment (H2O) or the parental strain (SYN-PKU901) (*, p<0.05; **, p<0.01; ***, p<0.001; ****, p<0.0001). FIGS. 77C and 77D depict scatter plots of the data shown in FIGS. 77A and 77B.

FIGS. 78A and 78B depict blood phenylalanine concentrations relative to baseline following subcutaneous phenylalanine challenge in an in vivo mouse model of PKU. Mice were orally gavaged with 200 μL of H2O (n=12), 200 μL of SYN-PKU901 (n=12), or 100, 200, or 400 μL of SYN-PKU304 (n=12 in each dose group) at 30 and 90 minutes post-phenylalanine injection (0.1 mg/gram of average group body weight). FIGS. 78A and 78B show a dose-dependent decrease in blood phenylalanine levels in SYN-PKU304-treated mice compared to mice administered mock treatment (H₂O) or the parental strain (SYN-PKU901) (* 30% decrease; p<0.05). This experiment represents one of eight studies of this same design, and each one shows that SYN-PKU304 is capable of intercepting enterorecirculating phenylalanine.

FIG. 79 depicts a bar graph showing the effect of pheP, various copy numbers of PAL, and the further addition of LAAD on the rate of phenylalanine degradation in vitro. Results demonstrate that increasing the copy number of PAL increases the rate of phenylalanine degradation. Addition of the high affinity transporter pheP abrogates the transport limitation, allowing greater PAL activity. The transporter copy number does not increase rate (PAL, and not transport (pheP), is limiting). In the presence of oxygen, LAAD can degrade Phe at an extremely high rate.

FIG. 80A, FIG. 80B, and FIG. 80C depict bar graphs showing measurements for characterization of the phenylalanine enterorecirculation model. PKU mice were maintained on Phe-free chow and were injected with phenylalanine subcutaneously (0.1 mg/kg body weight) at T=0. Blood was sampled at indicated timepoints to determine the kinetics of serum Phe post injection. The Whisker plots in FIG. 80A and FIG. 80B show distribution of mouse blood Phe levels, both overall Phe levels (FIG. 80A) and change in Phe levels from T0 (FIG. 80B). As seen in FIG. 80C, Phe levels are stably elevated over at least a 6 hour period.

FIG. 81 depicts a bar graph showing blood phenylalanine concentrations relative to baseline following subcutaneous phenylalanine challenge in an in vivo mouse model of PKU. Mice were orally gavaged three times with a total of 750 μL of H2O (n=9), SYN-PKU901 (n=9), or 800 μL of SYN-PKU707 (n=9) (1×10e11 cfu/mouse) at 1, 2, and 3 hours post-phenylalanine injection and blood and urine was collected 4 hours post injection. FIG. 81 depicts blood phenylalanine concentrations relative to baseline; total metabolic activity for SYN-PKU707 was calculated as 269 umol/hr and the total reduction in Δphe was =49% (P<0.05) relative to SYN-PKU901 (P<0.05).

FIG. 82A and FIG. 82B depict bar graphs showing the change in phenylalanine over baseline in blood (FIG. 57A) and the absolute levels of hippuric acid in urine (FIG. 57B) at 4 hours post phenylalanine challenge in PKU mice gavaged with SYN-PKU708 at the indicated doses. SYN-PKU708 was efficacious in reducing blood phenylalanine and hippurate was excreted in a dose dependent manner in the cages of mice treated with SYN-PKU708, indicating that the cells were active in vivo.

FIG. 83A, FIG. 83B, FIG. 83C, and FIG. 83D depicts schematics of exemplary embodiments of the disclosure, in which the genetically engineered bacteria comprise circuits for the production of tryptophan. Any of the gene(s), gene sequence(s) and/or gene circuit(s) or cassette(s) are optionally expressed from an inducible promoter. Exemplary inducible promoters which may control the expression of the gene(s), gene sequence(s) and/or gene circuit(s) or cassette(s) include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. The bacteria may also include an auxotrophy, e.g., deletion of thyA (A thyA; thymidine dependence). FIG. 83A shows a schematic depicting an exemplary Tryptophan circuit. Tryptophan is produced from its precursor, chorismate, through expression of the trpE, trpG-D (also referred to as trpD), trpC-F (also referred to as trpC), trpB and trpA genes. Optional knockout of the tryptophan repressor trpR is also depicted. Optional production of chorismate through expression of aroG/F/H and aroB, aroD, aroE, aroK and aroC genes is also shown. The bacteria may optionally also include gene sequence(s) for the expression of YddG, which functions as a tryptophan exporter. The bacteria may optionally also comprise one or more gene sequence(s) depicted or described in FIG. 83B, and/or FIG. 83C, and/or FIG. 83D. FIG. 83B depicts a tryptophan producing strain, in which tryptophan is produced from the chorismate precursor through expression of the trpE, trpG-D, trpC-F, trpB and trpA genes. AroG and TrpE are replaced with feedback resistant versions to improve tryptophan production. Optionally, bacteria may comprise any of the transporters and/or additional tryptophan circuits depicted in FIG. 83A and/or described in the description of FIG. 83A. The bacteria may optionally also comprise one or more gene sequence(s) depicted or described in FIG. 83C, and/or FIG. 83D. Optionally, trpR and/or the tnaA gene (encoding a tryptophanase converting tryptophan into indole) are deleted to further increase levels of tryptophan produced. FIG. 83C depicts a tryptophan producing strain, in which tryptophan is produced from the chorismate precursor through expression of the trpE, trpG-D, trpC-F, trpB and trpA genes. AroG and TrpE are replaced with feedback resistant versions to improve tryptophan production. The strain further comprises either a wild type or a feedback resistant SerA gene. Escherichia coli serA-encoded 3-phosphoglycerate (3PG) dehydrogenase catalyzes the first step of the major phosphorylated pathway of L-serine (Ser) biosynthesis. This step is an oxidation of 3PG to 3-phosphohydroxypyruvate (3PHP) with the concomitant reduction of NAD1 to NADH. E. coli uses one serine for each tryptophan produced. As a result, by expressing serA, tryptophan production is improved. Optionally, bacteria may comprise any of the transporters and/or additional tryptophan circuits depicted in FIG. 83A and/or described in the description of FIG. 83A. The bacteria may optionally also comprise one or more gene sequence(s) depicted or described in FIG. 83B, and/or FIG. 83D. Optionally, Trp Repressor and/or the tnaA gene are deleted to further increase levels of tryptophan produced. The bacteria may optionally also include gene sequence(s) for the expression of YddG, which functions as a tryptophan exporter. FIG. 83D depicts a non-limiting example of a tryptophan producing strain, in which tryptophan is produced from the chorismate precursor through expression of the trpE, trpG-D, trpC-F, trpB and trpA genes. AroG and TrpE are replaced with feedback resistant versions to improve tryptophan production. The strain further optionally comprises either a wild type or a feedback resistant SerA gene. Optionally, bacteria may comprise any of the transporters and/or additional tryptophan circuits depicted in FIG. 83A and/or described in the description of FIG. 83A. The bacteria may optionally also comprise one or more gene sequence(s) depicted or described in FIG. 83B, and/or FIG. 83C. Optionally, Trp Repressor and/or the tnaA gene are deleted to further increase levels of tryptophan produced. The bacteria may optionally also include gene sequence(s) for the expression of YddG, which functions as a tryptophan exporter. Optionally, the bacteria may also comprise a deletion in PheA, which prevents conversion of chorismate into phenylalanine and thereby promotes the production of anthranilate and tryptophan.

FIG. 84 depicts a schematic of the E. coli tryptophan synthesis pathway. In Escherichia coli, tryptophan is biosynthesized from chorismate, the principal common precursor of the aromatic amino acids tryptophan, tyrosine and phenylalanine, as well as the essential compounds tetrahydrofolate, ubiquinone-8, menaquinone-8 and enterobactin (enterochelin), as shown in the superpathway of chorismate metabolism. Five genes encode five enzymes that catalyze tryptophan biosynthesis from chorismate. The five genes trpE trpD trpC trpB trpA form a single transcription unit, the trp operon. A weak internal promoter also exists within the trpD structural gene that provides low, constitutive levels of mRNA.

FIG. 85 depicts a schematic of the trypophan catabolic pathway/indole biosynthesis pathways. Host and microbiota metabolites with AhR agonistic activity are in in diamond and circled, respectively (see, e.g., Lamas et al., CARDS impacts colitis by altering gut microbiota metabolism of tryptophan into aryl hydrocarbon receptor ligands; Nature Medicine 22, 598-605 (2016). In certain embodiments of the disclosure, the genetically engineered bacteria comprise gene cassettes comprising one or more of the bacterial tryptophan metabolism enzymes which catalyze the reactions shown in FIG. 85. In certain embodiments, the genetically engineered bacteria comprise one or more gene cassettes which produce one or more of the metabolites depicted in FIG. 85 including but not limited to, kynurenine, indole-3-aldehyde, indole-3-acetic acid, and/or indole-3 acetaldehyde.

FIG. 86A and FIG. 86B depict diagrams of bacterial tryptophan metabolism pathways. FIG. 86A depicts a schematic of the bacterial tryptophan metabolism, as described, e.g., in Enzymes are numbered as follows 1) Trp 2,3 dioxygenase (EC 1.13.11.11); 2) kynurenine formidase (EC 3.5.1.49); 3) kynureninase (EC 3.7.1.3); 4) tryptophanase (EC 4.1.99.1); 5) Trp aminotransferase (EC 2.6.1.27); 6) indole lactate dehydrogenase (EC1.1.1.110); 7) Trp decarboxylase (EC 4.1.1.28); 8) tryptamine oxidase (EC 1.4.3.4); 9) Trp side chain oxidase (EC 4.1.1.43); 10) indole acetaldehyde dehydrogenase (EC 1.2.1.3); 11) indole acetic acid oxidase; 13) Trp 2-monooxygenase (EC 1.13.12.3); and 14) indole acetamide hydrolase (EC 3.5.1.0). The dotted lines (—) indicate a spontaneous reaction. FIG. 86B Depicts a schematic of tryptophan derived pathways. Known AHR agonists are with asterisk. Abbreviations are as follows. Trp: Tryptophan; TrA: Tryptamine; IAAld: Indole-3-acetaldehyde; IAA: Indole-3-acetic acid; FICZ: 6-formylindolo(3,2-b)carbazole; IPyA: Indole-3-pyruvic acid; IAM: Indole-3-acetamine; IAOx: Indole-3-acetaldoxime; IAN: Indole-3-acetonitrile; N-formyl Kyn: N-formylkynurenine; Kyn:Kynurenine; KynA: Kynurenic acid; I3 C: Indole-3-carbinol; IAld: Indole-3-aldehyde; DIM: 3,3′-Diindolylmethane; ICZ: Indolo(3,2-b)carbazole. Enzymes are numbered as follows: 1. EC 1.13.11.11 (Tdo2, Bna2), EC 1.13.11.11 (Ido1); 2. EC 4.1.1.28 (Tdc); 3. EC 1.4.3.22, EC 1.4.3.4 (TynA); 4. EC 1.2.1.3 (lad1), EC 1.2.3.7 (Aao1); 5. EC 3.5.1.9 (Afmid Bna3); 6. EC 2.6.1.7 (Cclb1, Cclb2, Aadat, Got2); 7. EC 1.4.99.1 (TnaA); 8. EC 1.14.13.125 (CYP79B2, CYP79B3); 9. EC 1.4.3.2 (StaO), EC 2.6.1.27 (Aro9, aspC), EC 2.6.1.99 (Taa1), EC 1.4.1.19 (TrpDH); 10. EC 1.13.12.3 (laaM); 11. EC 4.1.1.74 (IpdC); 12. EC 1.14.13.168 (Yuc2); 13. EC 3.5.1.4 (IaaH); 14. EC 3.5.5.1. (Nit1); 15. EC 4.2.1.84 (Nit1); 16. EC 4.99.1.6 (CYP71A13); 17. EC 3.2.1.147 (Pent). In certain embodiments of the disclosure, the genetically engineered bacteria comprise gene cassettes comprising one or more of the bacterial tryptophan metabolism enzymes depicted in FIG. 96A and FIG. 86B. In certain embodiments, the genetically engineered bacteria comprise one or more gene cassettes which produce one or more of the metabolites depicted in FIG. 86A and FIG. 86B. In certain embodiments, the one or more cassettes are on a plasmid; in other embodiments, the cassettes are integrated into the genome. In certain embodiments the one or more cassettes are under the control of inducible promoters which are induced under low-oxygen conditions, in the presence of certain molecules or metabolites, in the presence of molecules or metabolites associated with inflammation or an inflammatory response, or in the presence of some other metabolite that may or may not be present in the gut, such as arabinose.

FIG. 87 depicts a schematic of one embodiment of the disclosure. In this embodiment, tryptophan is synthesized from kynurenine. Through this conversion, a immune-suppressive metabolite (kynurenine) can be removed from the external environment, e.g., a tumor environment, and a pro-inflammatory metabolite (tryptophan) is generated. Kynureninase from Pseudomonas fluorescens converts KYN to AA (Anthranillic acid), which then can be converted to tryptophan through the enzymes of the E. coli trp operon. Optionally, the trpE gene may be deleted as it is not needed for the generation of tryptophan from kynurenine. In alternate embodiments, the trpE gene is not deleted, in order to maximize tryptophan production by using both kynurenine and chorismate as a substrate. In one embodiment of the invention, the genetically engineered bacteria comprising this circuit may be useful for reducing immune escape in cancer. [[In another embodiment, the genetically engineered bacteria comprising this circuit may be useful in neurological applications. Kynureninase from Pseudomonas fluorescens preferentially uses KYN as a substrate over 3-HK, in contrast to human kynureninase, which prefers 3-HK over KYN. As a result, Pseudomonas fluorescens kynureninase expressed by the genetically engineered bacteria may be useful in diverting the catabolism of KYN from neurotoxic downstream metabolites, such as QUIN, to AA, which may lead to a beneficial change in KP metabolite ratios.]] In some embodiments, a new strain is generated through adaptive laboratory evolution. The ability of this strain to metabolize kynurenine is improved (through lowering of kynurenine substrate). Additionally, the ability or preference of the strain take up tryptophan is lowered (due to selection pressure imposed by toxic tryptophan analogs. As a result, this strain has improved therapeutic properties in a number of applications, including but not limited to immunoncology.

A figure (not shown) depicts a bar graph showing the kynurenine consumption rates of original and ALE evolved kynureninase expressing strains in M9 media supplemented with 75 uM kynurenine. Strains are labeled as follows: SYN1404: E. coli Nissle comprising a deletion in Trp:E and a medium copy plasmid expressing kynureninase from Pseudomonas fluorescens under the control of a tetracycline inducible promoter (Nissle delta TrpE::CmR+Ptet-Pseudomonas KYNU p15a KanR); SYN2027: E. coli Nissle comprising a deletion in Trp:E and expressing kynureninase from Pseudomonas fluorescens under the control of a constitutive promoter (the endogenous lpp promoter) integrated into the genome at the HA3/4 site (HA3/4::Plpp-pKYNase KanR TrpE::CmR); SYN2028: E. coli Nissle comprising a deletion in Trp:E and expressing kynureninase from Pseudomonas fluorescens under the control of a constitutive promoter (the synthetic J23119 promoter) integrated into the genome at the HA3/4 site (HA3/4::PSynJ23119-pKYNase KanR TrpE::CmR); SYN2027-R1: a first evolved strain resulting from ALE, derived from the parental SYN2027 strain (Plpp-pKYNase KanR TrpE::CmR EVOLVED STRAIN Replicate 1). SYN2027-R2: a second evolved strain resulting from ALE, derived from the parental SYN2027 strain (Plpp-pKYNase KanR TrpE::CmR EVOLVED STRAIN Replicate 2). SYN2028-R1: a first evolved strain resulting from ALE, derived from the parental SYN2028 strain (HA3/4::PSynJ23119-pKYNase KanR TrpE::CmR EVOLVED STRAIN Replicate 1). SYN2028-R2: a second evolved strain resulting from ALE, derived from the parental SYN2028 strain (HA3/4::PSynJ23119-pKYNase KanR TrpE::CmR EVOLVED STRAIN Replicate 1).

Figures (not shown) depict dot plots showing intratumoral kynurenine depletion by strains producing kynureninase from Pseudomonas fluorescens. The first figure depicts a dot plot showing a intra tumor concentrations observed for the kynurenine consuming strain SYN1704, carrying a constitutively expressed Pseudomonase fluorescens kynureninase on a medium copy plasmid. The second figure depicts a dot plot showing a intra tumor concentrations observed for the kynurenine consuming strain SYN2028 carrying a constitutively expressed chromosomally integrated copy of Pseudomonase fluorescens kynureninase. The IDO inhibitor INCB024360 is used as a positive control.

FIG. 88A, FIG. 88B, FIG. 88C, FIG. 88D, FIG. 88E, FIG. 88F, FIG. 88G, and FIG. 88H depict schematics of non-limiting examples of embodiments of the disclosure. In all embodiments, optionally gene(s) which encode exporters may also be included. FIG. 88A depicts one embodiment of the disclosure, in which the genetically engineered bacteria produce tryptamine from tryptophan. The bacteria may comprise any of the transporters and/or tryptophan circuits. Alternatively, optionally, tryptophan can be imported through a transporter. In addition, the genetically engineered bacteria comprise a circuit for Tryptophan decarboxylase, e.g., from Catharanthus roseus, which converts tryptophan to tryptamine, e.g., under the control of an inducible promoter e.g., an FNR promoter. FIG. 88B depicts one embodiment of the disclosure, in which the genetically engineered bacteria produce indole-3-acetaldehyde and FICZ from tryptophan. Alternatively, optionally, tryptophan can be imported through a transporter. In addition, the genetically engineered bacteria comprise a circuit for aro9 (L-tryptophan aminotransferase, e.g., from S. cerevisae) or aspC (aspartate aminotransferase, e.g., from E. coli, or taal (L-tryptophan-pyruvate aminotransferase, e.g., from Arabidopsis thaliana) or staO (L-tryptophan oxidase, e.g., from streptomyces sp. TP-A0274) or trpDH (Tryptophan dehydrogenase, e.g., from Nostoc punctiforme NIES-2108) and ipdC (Indole-3-pyruvate decarboxylase, e.g., from Enterobacter cloacae) which together produce indole-3-acetaldehyde and FICZ from tryptophan, e.g., under the control of an inducible promoter e.g., an FNR promoter. FIG. 88C depicts one embodiment of the disclosure, in which the genetically engineered bacteria produce indole-3-acetaldehyde and FICZ from tryptophan. In addition, the genetically engineered bacteria comprise a circuit comprising tdc (Tryptophan decarboxylase, e.g., from Catharanthus roseus or tdc from Clostridium sporogenes), and tynA (Monoamine oxidase, e.g., from E. coli), which converts tryptophan to indole-3-acetaldehyde and FICZ, e.g., under the control of an inducible promoter e.g., an FNR promoter. FIG. 88D depicts one embodiment of the disclosure, in which the genetically engineered bacteria produce indole-3-acetonitrile from tryptophan. Alternatively, optionally, tryptophan can be imported through a transporter. In addition, the genetically engineered bacteria comprise a circuit for cyp79B2, (tryptophan N-monooxygenase, e.g., from Arabidopsis thaliana) or cyp79B3 (tryptophan N-monooxygenase, e.g., from Arabidopsis thaliana), which together convert tryptophan to indole-3-acetonitrile, e.g., under the control of an inducible promoter e.g., an FNR promoter. FIG. 88E depicts one embodiment of the disclosure, in which the genetically engineered bacteria produce kynurenine from tryptophan. Alternatively, optionally, tryptophan can be imported through a transporter. In addition, the genetically engineered bacteria comprise a circuit comprising IDO1 (indoleamine 2,3-dioxygenase, e.g., from Homo sapiens or TDO2 (tryptophan 2,3-dioxygenase, e.g., from Homo sapiens) or BNA2 (indoleamine 2,3-dioxygenase, e.g., from S. cerevisiae) and Afmid: Kynurenine formamidase, e.g., from mouse) or BNA3 (kynurenine—oxoglutarate transaminase, e.g., from S. cerevisae) which together convert tryptophan to kynurenine, e.g., under the control of an inducible promoter e.g., an FNR promoter. FIG. 88F depicts one embodiment of the disclosure, in which the genetically engineered bacteria produce kynureninic acid from tryptophan. Alternatively, optionally, tryptophan can be imported through a transporter. In addition, the genetically engineered bacteria comprise a circuit comprising IDO1 (indoleamine 2,3-dioxygenase, e.g., from Homo sapiens or TDO2 (tryptophan 2,3-dioxygenase, e.g., from Homo sapiens) or BNA2 (indoleamine 2,3-dioxygenase, e.g., from S. cerevisiae) and Afmid: Kynurenine formamidase, e.g., from mouse) or BNA3 (kynurenine—oxoglutarate transaminase, e.g., from S. cerevisae) and GOT2 (Aspartate aminotransferase, mitochondrial, e.g., from Homo sapiens or AADAT (Kynurenine/alpha-aminoadipate aminotransferase, mitochondrial, e.g., from Homo sapiens), or CCLB1 (Kynurenine—oxoglutarate transaminase 1, e.g., from Homo sapiens) or CCLB2 (kynurenine—oxoglutarate transaminase 3, e.g., from Homo sapiens, which together produce kynureninic acid from tryptophan, under the control of an inducible promoter, e.g., an FNR promoter. FIG. 88G depicts one embodiment of the disclosure, in which the genetically engineered bacteria produce indole from tryptophan. Alternatively, optionally, tryptophan can be imported through a transporter. In addition, the genetically engineered bacteria comprise a circuit for tnaA (tryptophanase, e.g., from E. coli), which converts tryptophan to indole, e.g., under the control of an inducible promoter e.g., an FNR promoter. FIG. 88H depicts one embodiment of the disclosure, in which the genetically engineered bacteria produce indole-3-carbinol, indole-3-aldehyde, 3,3′ diindolylmethane (DIM), indolo(3,2-b) carbazole (ICZ) from indole glucosinolate taken up through the diet. The genetically engineered bacteria comprise a circuit comprising pne2 (myrosinase, e.g., from Arabidopsis thaliana) under the control of an inducible promoter, e.g. an FNR promoter. The engineered bacterium shown in any of FIG. 88A-H, may also have an auxotrophy, e.g., in one example, the thyA gene can be been mutated in the E. coli Nissle genome, so thymidine must be supplied in the culture medium to support growth.

FIG. 89A, FIG. 89B, FIG. 89C, FIG. 89D, and FIG. 89E depict schematics of exemplary embodiments of the disclosure, in which the genetically engineered bacteria convert tryptophan into indole-3-acetic acid. In FIG. 89A, Alternatively, optionally, tryptophan can be imported through a transporter. In addition, the genetically engineered bacteria comprise a circuit comprising aro9 (L-tryptophan aminotransferase, e.g., from S. cerevisae) or aspC (aspartate aminotransferase, e.g., from E. coli, or taal (L-tryptophan-pyruvate aminotransferase, e.g., from Arabidopsis thaliana) or staO (L-tryptophan oxidase, e.g., from streptomyces sp. TP-A0274) or trpDH (Tryptophan dehydrogenase, e.g., from Nostoc punctiforme NIES-2108) and ipdC (Indole-3-pyruvate decarboxylase, e.g., from Enterobacter cloacae) and iad1 (Indole-3-acetaldehyde dehydrogenase, e.g., from Ustilago maydis) or AAO1 (Indole-3-acetaldehyde oxidase, e.g., from Arabidopsis thaliana) which together produce indole-3-acetic acid from tryptophan, e.g., under the control of an inducible promoter e.g., an FNR promoter. In FIG. 89B Alternatively, optionally, tryptophan can be imported through a transporter. In addition, the genetically engineered bacteria comprise a circuit comprising tdc (Tryptophan decarboxylase, e.g., tdc from Catharanthus roseus or tdc from Clostridium sporogenes) of tynA (Monoamine oxidase, e.g., from E. coli) and or iad1 (Indole-3-acetaldehyde dehydrogenase, e.g., from Ustilago maydis) or AAO1 (Indole-3-acetaldehyde oxidase, e.g., from Arabidopsis thaliana), e.g., under the control of an inducible promoter e.g., an FNR promoter. In FIG. 89C Alternatively, optionally, tryptophan can be imported through a transporter. In addition, the genetically engineered bacteria comprise a circuit comprising aro9 (L-tryptophan aminotransferase, e.g., from S. cerevisae) or aspC (aspartate aminotransferase, e.g., from E. coli, or taal (L-tryptophan-pyruvate aminotransferase, e.g., from Arabidopsis thaliana) or staO (L-tryptophan oxidase, e.g., from streptomyces sp. TP-A0274) or trpDH (Tryptophan dehydrogenase, e.g., from Nostoc punctiforme NIES-2108) and yuc2 (indole-3-pyruvate monoxygenase, e.g., from Arabidopsis thaliana) e.g., under the control of an inducible promoter e.g., an FNR promoter. In FIG. 89D Alternatively, optionally, tryptophan can be imported through a transporter. In addition, the genetically engineered bacteria comprise a circuit comprising IaaM (Tryptophan 2-monooxygenase e.g., from Pseudomonas savastanoi) and iaaH (Indoleacetamide hydrolase, e.g., from Pseudomonas savastanoi), e.g., under the control of an inducible promoter e.g., an FNR promoter. In FIG. 89E Alternatively, optionally, tryptophan can be imported through a transporter. In addition, the genetically engineered bacteria comprise a circuit comprising cyp79B2 (tryptophan N-monooxygenase, e.g., from Arabidopsis thaliana) or cyp79B3 (tryptophan N-monooxygenase, e.g., from Arabidopsis thaliana and cyp71a13 (indoleacetaldoxime dehydratase, e.g., from Arabidopis thaliana) and nitl (Nitrilase, e.g., from Arabidopsis thaliana) and iaaH (Indoleacetamide hydrolase, e.g., from Pseudomonas savastanoi), e.g., under the control of an inducible promoter e.g., an FNR promoter. the engineered bacterium shown in any of FIG. 89A-E, may also have an auxotrophy, e.g., in one example, the thyA gene can be been mutated in the E. coli Nissle genome, so thymidine must be supplied in the culture medium to support growth.

FIG. 90A and FIG. 90B depict schematics of circuits for the production of indole metabolites. FIG. 90A depicts a schematic of an indole-3-propionic acid (IPA) synthesis circuit. IPA produced by the gut microbiota has a significant positive effect on barrier integrity. IPA does not signal through AhR, but rather through a different receptor (PXR) (Venkatesh et al., Symbiotic Bacterial Metabolites Regulate Gastrointestinal Barrier Function via the Xenobiotic Sensor PXR and Toll-like Receptor 4; Immunity 41, 296-310, Aug. 21, 2014, and US Patent Publication No. 20150258151). In some embodiments, IPA can be produced in a synthetic circuit by expressing two enzymes, a tryptophan ammonia lyase and an indole-3-acrylate reductase (e.g., Tryptophan ammonia lyase (WAL) (e.g., from Rubrivivax benzoatilyticus) and indole-3-acrylate reductase (e.g., from Clostridum botulinum). Tryptophan ammonia lyase converts tryptophan to indole-3-acrylic acid, and indole-3-acrylate reductase converts indole-3-acrylic acid into IPA. Without wishing to be bound by theory, no oxygen is needed for this reaction, allowing it to proceed under low or no oxygen conditions, e.g., as those found in the mammalian gut. FIG. 90B depicts a schematic of another indole-3-propionic acid (IPA) synthesis circuit. Enzymes are as follows: 1. TrpDH: tryptophan dehydrogenase, e.g., from Nostoc punctiforme NIES-2108; FldH1/FldH2: indole-3-lactate dehydrogenase, e.g., from Clostridium sporogenes; FldA: indole-3-propionyl-CoA:indole-3-lactate CoA transferase, e.g., from Clostridium sporogenes; FldBC: indole-3-lactate dehydratase, e.g., from Clostridium sporogenes; FldD: indole-3-acrylyl-CoA reductase, e.g., from Clostridium sporogenes; AcuI: acrylyl-CoA reductase, e.g., from Rhodobacter sphaeroides. Tryptophan dehydrogenase (EC 1.4.1.19) is an enzyme that catalyzes the reversible chemical reaction converting L-tryptophan, NAD(P) and water to indol-3-yl)pyruvate, NH₃, NAD(P)H and H⁺. Indole-3-lactate dehydrogenase ((EC 1.1.1.110, e.g., Clostridium sporogenes or Lactobacillus casei) converts (indol-3yl)pyruvate and NADH and H+ to indole-3-lactate and NAD+. Indole-3-propionyl-CoA:indole-3-lactate CoA transferase (FldA) converts indole-3-lactate and indol-3-propionyl-CoA to indole-3-propionic acid and indole-3-lactate-CoA. Indole-3-acrylyl-CoA reductase (FldD) and acrylyl-CoA reductase (AcuI) convert indole-3-acrylyl-CoA to indole-3-propionyl-CoA. Indole-3-lactate dehydratase (FldBC) converts indole-3-lactate-CoA to indole-3-acrylyl-CoA.

FIG. 91A and FIG. 91B depict schematics showing exemplary engineering strategies which can be employed for tryptophan production. FIG. 91A depicts a schematic showing intermediates in tryptophan biosynthesis and the gene products catalyzing the production of these intermediates. Phosphoenolpyruvate (PEP) and D-erythrose 4-phosphate (E4P) are used to generate 3-deoxy-D-arabino-heptulosonate 7-phosphate (DAHP). DHAP is catabolized to chorismate and then anthranilate, which is converted to tryptophan (Trp) by the tryptophan operon. Alternatively, chorismate can be used in the synthesis of tyrosine (Tyr) and/or phenylalanine (Phe). In the serine biosynthesis pathway, D-3-phosphoglycerate is converted to serine, which can also be a source for tryptophan biosynthesis. AroG, AroF, AroH: DAHP synthase catalyzes an aldol reaction between phosphoenolpyruvate and D-erythrose 4-phosphate to generate 3-deoxy-D-arabino-heptulosonate 7-phosphate (DAHP). There are three isozymes of DAHP synthase, each specifically feedback regulated by tyrosine (AroF), phenylalanine (AroG) or tryptophan(AroH). AroB: Dehydroquinate synthase (DHQ synthase) is involved in the second step of the chorismate pathway, which leads to the biosynthesis of aromatic amino acids. DHQ synthase catalyzes the cyclization of 3-deoxy-D-arabino-heptulosonic acid 7-phosphate (DAHP) to dehydroquinate (DHQ). AroD: 3-Dehydroquinate dehydratase (DHQ dehydratase) is involved in the 3rd step of the chorismate pathway, which leads to the biosynthesis of aromatic amino acids. DHQ dehydratase catalyzes the conversion of DHQ to 3-dehydroshikimate and introduces the first double bond of the aromatic ring. AroE, YdiB: E. coli expresses two shikimate dehydrogenase paralogs, AroE and YdiB Shikimate dehydrogenase is involved in the 4th step of the chorismate pathway, which leads to the biosynthesis of aromatic amino acids. This enzyme converts 3-dehydroshikimate to shikimate by catalyzing the NADPH linked reduction of 3-dehydro-shikimate AroL/AroK: Shikimate kinase is involved in the fifth step of the chorismate pathway, which leads to the biosynthesis of aromatic amino acids. Shikimate kinase catalyzes the formation of shikimate 3-phosphate from shikimate and ATP. There are two shikimate kinase enzymes, I (AroK) and II (AroL). AroA: 3-Phosphoshikimate-1-carboxyvinyltransferase (EPSP synthase) is involved in the 6th step of the chorismate pathway, which leads to the biosynthesis of aromatic amino acids. EPSP synthase catalyzes the transfer of the enolpyruvoyl moiety from phosphoenolpyruvate to the hydroxyl group of carbon 5 of shikimate 3-phosphate with the elimination of phosphate to produce 5-enolpyruvoyl shikimate 3-phosphate (EPSP). AroC: Chorismate synthase (AroC) is involved in the 7th and last step of the chorismate pathway, which leads to the biosynthesis of aromatic amino acids. This enzyme catalyzes the conversion of 5-enolpyruvylshikimate 3-phosphate into chorismate, which is the branch point compound that serves as the starting substrate for the three terminal pathways of aromatic amino acid biosynthesis. This reaction introduces a second double bond into the aromatic ring system. TrpEDCAB (E. coli trp operon): TrpE (anthranilate synthase) converts chorismate and L-glutamine into anthranilate, pyruvate and L-glutamate. Anthranilate phosphoribosyl transferase (TrpD) catalyzes the second step in the pathway of tryptophan biosynthesis. TrpD catalyzes a phosphoribosyltransferase reaction that generates N-(5′-phosphoribosyl)-anthranilate. The phosphoribosyl transferase and anthranilate synthase contributing portions of TrpD are present in different portions of the protein. Bifunctional phosphoribosylanthranilate isomerase/indole-3-glycerol phosphate synthase (TrpC) carries out the third and fourth steps in the tryptophan biosynthesis pathway. The phosphoribosylanthranilate isomerase activity of TrpC catalyzes the Amadori rearrangement of its substrate into carboxyphenylaminodeoxyribulose phosphate. The indole-glycerol phosphate synthase activity of TrpC catalyzes the ring closure of this product to yield indole-3-glycerol phosphate. The TrpA polypeptide (TSase α) functions as the a subunit of the tetrameric (α2-β2) tryptophan synthase complex. The TrpB polypeptide functions as the β subunit of the complex, which catalyzes the synthesis of L-tryptophan from indole and L-serine, also termed the β reaction. TnaA: Tryptophanase or tryptophan indole-lyase (TnaA) is a pyridoxal phosphate (PLP)-dependent enzyme that catalyzes the cleavage of L-tryptophan to indole, pyruvate and NH4+. PheA: Bifunctional chorismate mutase/prephenate dehydratase (PheA) carries out the shared first step in the parallel biosynthetic pathways for the aromatic amino acids tyrosine and phenylalanine, as well as the second step in phenylalanine biosynthesis. TyrA: Bifunctional chorismate mutase/prephenate dehydrogenase (TyrA) carries out the shared first step in the parallel biosynthetic pathways for the aromatic amino acids tyrosine and phenylalanine, as well as the second step in tyrosine biosynthesis. TyrB, ilvE, AspC: Tyrosine aminotransferase (TyrB), also known as aromatic-amino acid aminotransferase, is a broad-specificity enzyme that catalyzes the final step in tyrosine, leucine, and phenylalanine biosynthesis. TyrB catalyzes the transamination of 2-ketoisocaproate, p-hydroxyphenylpyruvate, and phenylpyruvate to yield leucine, tyrosine, and phenylalanine, respectively. TyrB overlaps with the catalytic activities of branched-chain amino-acid aminotransferase (IlvE), which also produces leucine, and aspartate aminotransferase, PLP-dependent (AspC), which also produces phenylalanine. SerA: D-3-phosphoglycerate dehydrogenase catalyzes the first committed step in the biosynthesis of L-serine. SerC: The serC-encoded enzyme, phosphoserine/phosphohydroxythreonine aminotransferase, functions in the biosythesis of both serine and pyridoxine, by using different substrates. Pyridoxal 5′-phosphate is a cofactor for both enzyme activities. SerB: Phosphoserine phosphatase catalyzes the last step in serine biosynthesis. Steps which are negatively regulated by the Trp Repressor (2), Tyr Repressor (1), or tyrosine (3), phenylalanine (4), or tryptophan (4) or positively regulated by trptophan (6) are indicated. FIG. 91B depicts a schematic showing exemplary engineering strategies which can improve tryptophan production. Each of these exemplary strategies can be used alone or two or more strategies can be combined to increase tryptophan production. Intervention points are in bold, italics and underlined. In one embodiment of the disclosure, bacteria are engineered to express a feedback resistant from of AroG (AroGfbr). In one embodiment, bacteria are engineered to express AroL. In one embodiment, bacteria are engineered to comprise one or more copies of a feedback resistant form of TrpE (TrpEfbr). In one embodiment, bacteria are engineered to comprise one or more additional copies of the Trp operon, e.g., TrpE, e.g. TrpEfbr, and/or TrpD, and/or TrpC, and/or TrpA, and/or TrpB. In one embodiment, endogenous TnaA is knocked out through mutation(s) and/or deletion(s). In one embodiment, bacteria are engineered to comprise one or more additional copies of SerA. In one embodiment, bacteria are engineered to comprise one or more additional copies of YddG, a tryptophan exporter. In one embodiment, endogenous PheA is knocked out through mutation(s) and/or deletion(s). In one embodiment, bacteria are engineered to comprise a circuit for the expression of kynureninase, e.g., kynureninase from Pseudomonas fluorescens or human kynureninase, Without wishing to be bound by theory, addition of a circuit expressing kynureninase will increase production of tryptophan if kynurenine is present in the extracellular environment, such as for example a tumor microenvironment. A strain comprising circuitry to enhance tryptophan production and circuitry for the consumption of kynurenine reduces kynurenine levels while increasing tryptophan levels, e.g., in the extracellular environment, such as a tumor microenvironment, thereby more effectively changing the tryptophan to kynurenine ratio. In one embodiment, two or more of the strategies depicted in the schematic of FIG. 93B are engineered into a bacterial strain. Alternatively, other gene products in this pathway may be mutated or overexpressed.

FIG. 92 depicts schematics of exemplary embodiments of the disclosure, in which the genetically engineered bacteria comprise circuits for the production of tryptophan and the degradation of kynurenine. Any of the gene(s), gene sequence(s) and/or gene circuit(s) or cassette(s) are optionally expressed from an inducible promoter. Exemplary inducible promoters which may control the expression of the gene(s), gene sequence(s) and/or gene circuit(s) or cassette(s) include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. The bacteria may also include an auxotrophy, e.g., deletion of thyA (A thyA; thymidine dependence). =In one embodiment, the tryptophan is produced from the chorismate precursor through expression of the trpE, trpG-D, trpC-F, trpB and trpA genes. Optionally, Trp Repressor and/or the tnaA gene (encoding a tryptophanase converting tryptophan into indole) are deleted to further increase levels of tryptophan produced. Additionally, AroG and TrpE are replaced with feedback resistant versions to improve tryptophan production, and the strain further optionally comprises either a wild type or a feedback resistant serA gene. The bacteria may also optionally include gene sequence(s) for the expression of YddG to assist in tryptophan export. Additionally, the bacteria further comprise kynureninase, e.g., kynureninase from Pseudomonas fluorescens. When extracellular kynurenine is present, it is imported into the cell and is then converted by kynureninase into anthranilate. Anthranilate is then metabolized into tryptophan via the TrpDCAB pathway enzymes, resulting in further increased levels of tryptophan production.

FIG. 93A, FIG. 93B, and FIG. 93C depict schematics of exemplary embodiments of the disclosure, in which the genetically engineered bacteria comprise circuits for the production of tryptophan, tryptamine, indole acetic acid, and indole propionic acid. Any of the gene(s), gene sequence(s) and/or gene circuit(s) or cassette(s) are optionally expressed from an inducible promoter. Exemplary inducible promoters which may control the expression of the gene(s), gene sequence(s) and/or gene circuit(s) or cassette(s) include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. The bacteria may also include an auxotrophy, e.g., deletion of thyA (Δ thyA; thymidine dependence). FIG. 93A a depicts non-limiting example of a tryptamine producing strain. Additionally, the strain comprises tdc (tryptophan decarboxylase, e.g., from Catharanthus roseus or tdc from Clostridium sporogenes), which converts tryptophan into tryptamine FIG. 93B depicts a non-limiting example of an indole-3-acetate producing strain. Additionally, the strain comprises trpDH (Tryptophan dehydrogenase, e.g., from Nostoc punctiforme NIES-2108) and ipdC (Indole-3-pyruvate decarboxylase, e.g., from Enterobacter cloacae) which together produce indole-3-acetaldehyde and FICZ though an (indol-3yl)pyruvate intermediate, and iad1 (Indole-3-acetaldehyde dehydrogenase, e.g., from Ustilago maydis), which converts indole-3-acetaldehyde into indole-3-acetate. FIG. 93C depicts a non-limiting example of an indole-3-propionate-producing strain. Additionally, the strain comprises a circuit as described, comprising trpDH (Tryptophan dehydrogenase, e.g., from Nostoc punctiforme NIES-2108, which produces (indol-3yl)pyruvate from tryptophan), fldA (indole-3-propionyl-CoA:indole-3-lactate CoA transferase, e.g., from Clostridium sporogenes, which converts converts indole-3-lactate and indol-3-propionyl-CoA to indole-3-propionic acid and indole-3-lactate-CoA), fldB and fldC (indole-3-lactate dehydratase e.g., from Clostridium sporogenes, which converts indole-3-lactate-CoA to indole-3-acrylyl-CoA) fldD and/or AcuI: (indole-3-acrylyl-CoA reductase, e.g., from Clostridium sporogenes and/or acrylyl-CoA reductase, e.g., from Rhodobacter sphaeroides, which convert indole-3-acrylyl-CoA to indole-3-propionyl-CoA). The circuits further comprise fldH1 and/or fldH2 (indole-3-lactate dehydrogenase 1 and/or 2, e.g., from Clostridium sporogenes), which converts (indol-3-yl)pyruvate into indole-3-lactate).

FIG. 94A, FIG. 95B, FIG. 95C, and FIG. 95D depict bar graphs showing tryptophan production by various engineered bacterial strains. FIG. 94A depicts a bar graph showing tryptophan production by various tryptophan producing strains. The data show expressing a feedback resistant form of AroG (AroG^{fbr}) is necessary to get tryptophan production. Additionally, using a feedback resistant trpE (trpE^{fbr}) has a positive effect on tryptophan production. FIG. 95B shows tryptophan production from a strain comprising a tet-trpE^{fbr}DCBA, tet-aroG^{fbr} construct, comparing glucose and glucuronate as carbon sources in the presence and absence of oxygen. It takes E. coli two molecules of phosphoenolpyruvate (PEP) to produce one molecule of tryptophan. When glucose is used as the carbon source, 50% of all available PEP is used to import glucose into the cell through the PTS system (Phosphotransferase system). Tryptophan production is improved by using a non-PTS sugar (glucuronate) aerobically. The data also show the positive effect of deleting tnaA (only at early time point aerobically). FIG. 95C depicts a bar graph showing improved tryptophan production by engineered strain comprising ΔtrpRΔtnaA, tet-trpE^{fbr}DCBA, tet-aroG^{fbr} through the addition of serine. FIG. 95D depicts a bar graph showing a comparison in tryptophan production in strains SYN2126, SYN2323, SYN2339, SYN2473, and SYN2476. SYN2126 ΔtrpRΔtnaA. ΔtrpRΔtnaA, tet-aroGfbr. SYN2339 comprises ΔtrpRΔtnaA, tet-aroGfbr, tet-trpEfbrDCBA. SYN2473 comprises ΔtrpRΔtnaA, tet-aroGfbr-serA, tet-trpEfbrDCBA. SYN2476 comprises ΔtrpRΔtnaA, tet-trpEfbrDCBA. Results indicate that expressing aroG is not sufficient nor necessary under these conditions to get Trp production and that expressing serA is beneficial for tryptophan production.

FIG. 96A depicts a bar graph showing tryptophan and indole acetic acid production for strains SYN2126, SYN2339 and SYN2342. SYN2126: comprises ΔtrpR and ΔtnaA (ΔtrpRΔtnaA). SYN2339 comprises circuitry for the production of tryptophan (ΔtrpRΔtnaA, tetR-Ptet-trpEfbrDCBA (pSC101), tetR-Ptet-aroGfbr (p15A)). SYN2342 comprises the same tryptophan production circuitry as the parental strain SYN2339, and additionally comprises ipdC-iad1 incorporated at the end of the second construct (ΔtrpRΔtnaA, tetR-Ptet-trpEfbrDCBA (pSC101), tetR-Ptet-aroGfbr-trpDH-ipdC-iad1 (p15A)). SYN2126 produced no tryptophan, SYN2339 produces increasing tryptophan over the time points measured, and SYN2342 converts all trypophan it produces into IAA.

FIG. 96B depicts a bar graph showing tryptophan and tryptamine production for strains SYN2339, SYN2340, and SYN2794. SYN2339 is used as a control which can produce tryptophan but cannot convert it to tryptamine and comprises ΔtrpRΔtnaA, tetR-Pₜₑₜ-trpE^{fbr}DCBA (pSC101), tetR-Pₜₑₜ-aroG^{fbr} (p15A). SYN2340 comprises ΔtrpRΔtnaA, tetR-Pₜₑₜ-trpE^{fbr}DCBA (pSC101), tetR-Pₜₑₜ-aroG^{fbr}-tdc_{Cr} (p15A). SYN2794 comprises ΔtrpRΔtnaA, tetR-Pₜₑₜ-trpE^{fbr}DCBA (pSC101), tetR-Pₜₑₜ-aroG^{fbr}-tdc_{Cs} (p15A). Results indicate that Tdc_{Cs} from Clostridium sporogenes is more efficient the Tdc_{Cr} from Catharanthus roseus in tryptamine production and converts all the tryptophan produced into tryptamine

### DETAILED DESCRIPTION

The present disclosure provides recombinant bacterial cells that have been engineered with genetic circuitry which allow the recombinant bacterial cells to sense a patient's internal environment and respond by turning an engineered metabolic pathway on or off. When turned on, the recombinant bacterial cells complete all of the steps in a metabolic pathway to achieve a therapeutic effect in a host subject and are designed to drive therapeutic effects throughout the body of a host from a point of origin of the microbiome.

Specifically, the present disclosure provides recombinant bacterial cells, pharmaceutical compositions thereof, and methods of modulating and treating diseases associated with amino acid metabolism, such as cancer. Specifically, the recombinant bacteria disclosed herein have been constructed to comprise genetic circuits composed of, for example, an amino acid catabolism enzyme to treat cancer, as well as other circuitry in order to guarantee the safety and non-colonization of the subject that is administered the recombinant bacteria, such as auxotrophies, kill switches, etc. These recombinant bacteria are safe and well tolerated and augment the innate activities of the subject's microbiome to achieve a therapeutic effect.

In some embodiments, a bacterial cell disclosed herein has been genetically engineered to comprise a heterologous gene sequence encodingone or more amino acid catabolism enzymes and is capable of processing (e.g., metabolizing) and reducing levels of amino acid(s). In some embodiments, a bacterial cell disclosed herein has been genetically engineered to comprise a heterologous gene sequence encoding one or more amino acid catabolism enzymes and is capable of processing and reducing levels of amino acid(s) in low-oxygen environments, e.g., the gut. Thus, the genetically engineered bacterial cells and pharmaceutical compositions comprising the bacterial cells disclosed herein may be used to convert excess amino acids into non-toxic molecules in order to treat and/or prevent diseases associated with amino acid metabolism, such as cancer. In some embodiments, a bacterial cell disclosed herein has been genetically engineered to comprise a heterologous gene sequence encoding one or more amino acid biosynthesis enzymes and is capable of producing an amino acid. In some embodiments, a bacterial cell disclosed herein has been genetically engineered to comprise a heterologous gene sequence encoding one or more amino acid biosynthesis enzymes and is capable of producing an amino acid in low-oxygen environments, e.g., the gut. Thus, the genetically engineered bacterial cells and pharmaceutical compositions comprising the bacterial cells disclosed herein may be used to produce an amino acid in order to treat and/or prevent diseases associated with amino acid metabolism, such as cancer.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells of the disclosure may comprise exogenous nucleotide sequences on plasmids. Alternatively, recombinant bacterial cells may comprise exogenous nucleotide sequences stably incorporated into their chromosome.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature. As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures. In one embodiment, the regulatory sequence comprises a promoter, e.g., an FNR responsive promoter.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding at least one amino acid catabolism enzyme, that is joined to a regulatory sequence in a manner which allows expression of the nucleic acid sequence, e.g., the gene(s) encoding the amino acid catabolism enzyme. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive.

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Examples of inducible promoters include, but are not limited to, an FNR promoter, a P_{araC} promoter, a P_{araBAD} promoter, a propionate promoter, and a P_{TetR} promoter, each of which are described in more detail herein. Examples of other inducible promoters are provided herein below.

As used herein, “stably maintained” or “stable” bacterium is used to refer to a bacterial host cell carrying non-native genetic material, e.g., an amino acid catabolism enzyme, that is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising an amino acid catabolism gene, in which the plasmid or chromosome carrying the amino acid catabolism gene is stably maintained in the bacterium, such that the amino acid catabolism enzyme can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding at least one amino acid catabolism enzyme.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising at least one amino acid catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of an asparagine. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

“Exogenous environmental condition(s)” or “environmental conditions” refer to settings or circumstances under which the promoter described herein is directly or indirectly induced. The phrase is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions refer to the presence of molecules or metabolites that are specific to the mammalian gut in a healthy or disease-state, e.g., propionate. In some embodiments, the exogenous environmental condition is a tissue-specific or disease-specific metabolite or molecule(s). In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics.

As used herein, “exogenous environmental conditions” or “environmental conditions” also refers to settings or circumstances or environmental conditions external to the engineered microorganism, which relate to in vitro culture conditions of the microorganism. “Exogenous environmental conditions” may also refer to the conditions during growth, production, and manufacture of the organism. Such conditions include aerobic culture conditions, anaerobic culture conditions, low oxygen culture conditions and other conditions under set oxygen concentrations. Such conditions also include the presence of a chemical and/or nutritional inducer, such as tetracycline, arabinose, IPTG, rhamnose, and the like in the culture medium. Such conditions also include the temperatures at which the microorganisms are grown prior to in vivo administration. For example, using certain promoter systems, certain temperatures are permissive to expression of a payload, while other temperatures are non-permissive. Oxygen levels, temperature and media composition influence such exogenous environmental conditions. Such conditions affect proliferation rate, rate of induction of the payload or gene of interest, e.g., amino acid catabolism gene, other regulators (e.g., FNRS24Y), and overall viability and metabolic activity of the strain during strain production.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut).

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003). Non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic and/or low oxygen conditions by the global transcriptional regulator FNR that is naturally found in Nissle. Under anaerobic and/or low oxygen conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 1
Examples of transcription factors and
responsive genes and regulatory regions

| | Examples of responsive genes, |
|---|---|
| Transcription factor | promoters, and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions. In some embodiments, the genetically engineered bacteria of the invention comprise a gene encoding a phenylalanine-metabolizing enzyme that is operably linked to a directly or indirectly inducible promoter that is not associated with said gene in nature, e.g., an FNR promoter operably linked to a gene encoding an amino acid metabolism gene.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

In some embodiments, the genetically engineered bacteria are active in the gut. In some embodiments, the genetically engineered bacteria are active in the large intestine. In some embodiments, the genetically engineered bacteria are active in the small intestine. In some embodiments, the genetically engineered bacteria are active in the small intestine and in the large intestine. In some embodiments, the genetically engineered bacteria transit through the small intestine. In some embodiments, the genetically engineered bacteria have increased residence time in the small intestine. In some embodiments, the genetically engineered bacteria colonize the small intestine. In some embodiments, the genetically engineered bacteria do not colonize the small intestine. In some embodiments, the genetically engineered bacteria have increased residence time in the gut. In some embodiments, the genetically engineered bacteria colonize the small intestigutne. In some embodiments, the genetically engineered bacteria do not colonize the gut.

As used herein, the term “low oxygen” is meant to refer to a level, amount, or concentration of oxygen (O₂) that is lower than the level, amount, or concentration of oxygen that is present in the atmosphere (e.g., <21% O₂, <160 torr O₂)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorportated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table A summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring.) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE A

| Compartment | Oxygen Tension |
|---|---|
| stomach | ~60 torr (e.g., 58 +/− 15 torr) |
| duodenum and first | ~30 torr (e.g., 32 +/− 8 torr); ~20% oxygen in |
| part of jejunum | ambient air |
| Ileum (mid-small | ~10 torr; ~6% oxygen in ambient air (e.g., 11 +/− 3 |
| intestine) | torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, yeast, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment, e.g., the gut. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to, Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., amino acid metabolism gene, which is incorporated into the host genome or propagated on a self-replicating extra-chromosomal plasmid, such that the non-native genetic material is retained, expressed, and/or propagated. The stable bacterium is capable of survival and/or growth in vitro, e.g., in medium, and/or in vivo, e.g., in the gut. For example, the stable bacterium may be a genetically modified bacterium comprising an amino acid metabolism gene, in which the plasmid or chromosome carrying the amino acid metabolism gene is stably maintained in the host cell, such that amino acid metabolism gene can be expressed in the host cell, and the host cell is capable of survival and/or growth in vitro and/or in vivo. In some embodiments, copy number affects the stability of expression of the non-native genetic material, e.g., a amino acid metabolism gene. In some embodiments, copy number affects the level of expression of the non-native genetic material, e.g., amino acid metabolism gene.

As used herein, the term “auxotroph” or “auxotrophic” refers to an organism that requires a specific factor, e.g., an amino acid, a sugar, or other nutrient, to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Disorders associated with or involved with amino acid metabolism, e.g., cancer, may be caused by inborn genetic mutations for which there are no known cures. Diseases can also be secondary to other conditions, e.g., an intestinal disorder or a bacterial infection. Treating diseases associated with amino acid metabolism may encompass reducing normal levels of one or more amino acids, reducing excess levels of one or more amino acids, or eliminating one or more amino acids, and does not necessarily encompass the elimination of the underlying disease.

As used herein the terms “disease associated with amino acid metabolism” or a “disorder associated with amino acid metabolism” is a disease or disorder involving the abnormal, e.g., increased, levels of one or more amino acids in a subject. In one embodiment, a disease or disorder associated with amino acid metabolism is a cancer. In another embodiment, a disease or disorder associated with amino acid metabolism is a metabolic disease. In one embodiment, the cancer is glioma. In another embodiment, the cancer is breast cancer. In another embodiment, the cancer is melanoma. In another embodiment, the cancer is hepatocarcinoma. In another embodiment, the cancer is acute lymphoblastic leukemia (ALL). In another embodiment, the cancer is ovarian cancer. In another embodiment, the cancer is prostate cancer. In another embodiment, the cancer is lymphoblastic leukemia. In another embodiment, the cancer is non-small cell lung cancer.

As used herein, the term “amino acid” refers to a class of organic compounds that contain at least one amino group and one carboxyl group Amino acids include leucine, isoleucine, valine, arginine, lysine, asparagine, serine, glycine, glutamine, tryptophan, methionine, threonine, cysteine, tyrosine, phenylalanine, glutamic acid, aspartic acid, alanine, histidine, and proline.

As used herein, the term “amino acid catabolism” or “amino acid metabolism” refers to the processing, breakdown and/or degradation of an amino acid molecule (e.g., asparagine, lysine or arginine) into other compounds that are not associated with the disease associated with amino acid metabolism, such as cancer, or other compounds which can be utilized by the bacterial cell.

In one embodiment, the term “amino acid catabolism” refers to the processing, breakdown, and/or degradation of lysine into saccharopine. In another embodiment, the term “amino acid catabolism” refers to the processing, breakdown, and/or degradation of serine into 2-aminoprop-2-enoate. In yet another embodiment, the term “amino acid catabolism” refers to the processing, breakdown, and/or degradation of glutamine into ammonium and glutamate. In one embodiment, the term “amino acid catabolism” refers to the processing, breakdown, and/or degradation of tryptophan into indole-3-pyruvate.

In another embodiment, the term “amino acid catabolism” refers to the processing, breakdown, and/or degradation of methionine into S-adenosyl-L-homocysteine. In yet another embodiment, the term “amino acid catabolism” refers to the processing, breakdown, and/or degradation of methionine to sulfate. In one embodiment, the term “amino acid catabolism” refers to the processing, breakdown, and/or degradation of methionine into methanethiol and 2-aminobut-2-enoate. In another embodiment, the term “amino acid catabolism” refers to the processing, breakdown, and/or degradation of methionine into 3-methylthio-2-oxobutyric acid.

In yet another embodiment, the term “amino acid catabolism refers to the processing, breakdown, and/or degradation of cysteine into cystathione. In one embodiment, the term “amino acid catabolism” refers to the processing, breakdown, and/or degradation of threonine into amino-ketobutyrate. In another embodiment, the term “amino acid catabolism” refers to the processing, breakdown, and/or degradation of threonine into glycine and acetaldehyde. In yet another embodiment, the term “amino acid catabolism” refers to the processing, breakdown, and/or degradation of serine into glycine. In one embodiment, the term “amino acid catabolism” refers to the processing, breakdown, and/or degradation of cysteine into sulfide, NH₃ and pyruvate.

In another embodiment, the term “amino acid catabolism” refers to the processing, breakdown, and/or degradation of leucine into its respective acyl-CoA derivative. In another embodiment, the term “amino acid catabolism” refers to the processing, breakdown, and/or degradation of leucine into isobutyraldehyde. In another embodiment, the term “amino acid catabolism” refers to the processing, breakdown, and/or degradation of isoleucine into its corresponding α-keto acid counterpart and/or its acyl-CoA counterpart. In another embodiment, the term “amino acid catabolism” refers to the processing, breakdown, and/or degradation of valine into its corresponding α-keto acid counterpart and/or its acyl-CoA counterpart. In another embodiment, the term “amino acid catabolism” refers to the processing, breakdown, and/or degradation of arginine into agmatine.

In another embodiment, the term “amino acid catabolism” refers to the processing, breakdown, and/or degradation of asparagine into aspartic acid. In another embodiment, the term “amino acid catabolism” refers to the processing, breakdown, and/or degradation of tyrosine into glutamate. In another embodiment, the term “amino acid catabolism” refers to the processing, breakdown, and/or degradation of phenylalanine into trans-cinammic acid, ammonia, and/or tyrosine. In another embodiment, the term “amino acid catabolism” refers to the processing, breakdown, and/or degradation of glutamic acid into γ-Aminobutyric acid (GABA). In another embodiment, the term “amino acid catabolism” refers to the processing, breakdown, and/or degradation of histidine into glutamate. In another embodiment, the term “amino acid catabolism” refers to the processing, breakdown, and/or degradation of proline into 5-aminovalerate.

As used herein, the term “transporter” is meant to refer to a mechanism, e.g., protein, proteins, or protein complex, for importing a molecule, e.g., amino acid, peptide (di-peptide, tri-peptide, polypeptide, etc), toxin, metabolite, substrate, as well as other biomolecules into the microorganism from the extracellular milieu.

As used herein, “payload” refers to one or more molecules of interest to be produced by a genetically engineered microorganism, such as a bacteria or a virus. In some embodiments, the payload is a therapeutic payload, e.g., an amino acid catabolic enzyme or an amino acid transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments the payload comprises a repressor element, such as a kill switch. In some embodiments, the payload is encoded by a gene or multiple genes or an operon. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition. A therapeutically effective amount may, for example, be sufficient to treat, prevent, reduce the severity, delay the onset, and/or reduce the risk of occurrence of one or more symptoms of a disease or condition associated with excess amino acid levels. A therapeutically effective amount, as well as a therapeutically effective frequency of administration, can be determined by methods known in the art and discussed below.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids, Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (hut not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. Arm amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785, For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60 at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. The term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. In some embodiments, the improvement of transcription and/or translation involves increasing the level of transcription and/or translation. In some embodiments, the improvement of transcription and/or translation involves decreasing the level of transcription and/or translation. In some embodiments, codon optimization is used to fine-tune the levels of expression from a construct of interest, e.g., PAL3 levels and/or PheP levels. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent, inter alia, on the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting the protein(s) of interest or therapeutic protein(s) from the microbial, e.g., bacterial cytoplasm. The secretion system may comprise a single protein or may comprise two or more proteins assembled in a complex e.g., HlyBD. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the proteins of interest include a “secretion tag” of either RNA or peptide origin to direct the protein(s) of interest or therapeutic protein(s) to specific secretion systems. In some embodiments, the secretion system is able to remove this tag before secreting the protein(s) of interest from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the protein(s) of interest into the extracellular milieu.]]

As used herein, the term “transporter” is meant to refer to a mechanism, e.g., protein or proteins, for importing a molecule, e.g., amino acid, toxin, metabolite, substrate, etc. into the microorganism from the extracellular milieu. For example, a phenylalanine transporter such as PheP imports phenylalanine into the microorganism.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein a “pharmaceutical composition” refers to a preparation of bacterial cells disclosed herein with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The articles “a” and “an,” as used herein, should be understood to mean “at least one,” unless clearly indicated to the contrary. For example, as used herein, “a heterologous gene encoding an amino acid catabolism enzyme” should be understood to mean “at least one heterologous gene encoding at least one amino acid catabolism enzyme.” Similarly, as used herein, “a heterologous gene encoding an amino acid transporter” should be understood to mean “at least one heterologous gene encoding at least one amino acid transporter.”

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Bacterial Strains

The disclosure provides a bacterial cell that comprises a heterologous gene encoding an amino acid catabolism enzyme. In some embodiments, the bacterial cell is a non-pathogenic bacterial cell. In some embodiments, the bacterial cell is a commensal bacterial cell. In some embodiments, the bacterial cell is a probiotic bacterial cell.

In certain embodiments, the bacterial cell is selected from the group consisting of a Bacteroides fragilis, Bacteroides thetaiotaomicron, Bacteroides subtilis, Bifidobacterium animalis, Bifidobacterium bifidum, Bifidobacterium infantis, Bifidobacterium lactis, Clostridium butyricum, Clostridium scindens, Escherichia coli, Lactobacillus acidophilus, Lactobacillus plantarum, Lactobacillus reuteri, Lactococcus lactis, and Oxalobacter formigenes bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium animalis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is a Clostridium scindens bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a Oxalobacter formigenes bacterial cell. In another embodiment, the bacterial cell does not include Oxalobacter formigenes.

In one embodiment, the bacterial cell is a Gram positive bacterial cell. In another embodiment, the bacterial cell is a Gram negative bacterial cell.

In some embodiments, the bacterial cell is Escherichia coli strain Nissle 1917 (E. coli Nissle), a Gram-positive bacterium of the Enterobacteriaceae family that “has evolved into one of the best characterized probiotics” (Ukena et al., 2007). The strain is characterized by its “complete harmlessness” (Schultz, 2008), and “has GRAS (generally recognized as safe) status” (Reister et al., 2014, emphasis added). Genomic sequencing confirmed that E. coli Nissle “lacks prominent virulence factors (e.g., E. coli α-hemolysin, P-fimbrial adhesins)” (Schultz, 2008), and E. coli Nissle “does not carry pathogenic adhesion factors and does not produce any enterotoxins or cytotoxins, it is not invasive, not uropathogenic” (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's “therapeutic efficacy and safety have convincingly been proven” (Ukena et al., 2007).

In one embodiment, the recombinant bacterial cell of the disclosure does not colonize the subject having cancer.

One of ordinary skill in the art would appreciate that the genetic modifications disclosed herein may be adapted for other species, strains, and subtypes of bacteria. Furthermore, genes from one or more different species can be introduced into one another, e.g., a amino acid catabolism gene from Klebsiella quasipneumoniae can be expressed in Escherichia coli.

In some embodiments, the bacterial cell is a genetically engineered bacterial cell. In another embodiment, the bacterial cell is a recombinant bacterial cell. In some embodiments, the disclosure comprises a colony of bacterial cells.

In another aspect, the disclosure provides a recombinant bacterial culture which comprises bacterial cells disclosed herein. In one aspect, the disclosure provides a recombinant bacterial culture which reduces levels of an amino acid, e.g., asparagine, in the media of the culture. In one embodiment, the levels of an amino acid are reduced by about 50%, about 75%, or about 100% in the media of the cell culture. In another embodiment, the levels of an amino acid are reduced by about two-fold, three-fold, four-fold, five-fold, six-fold, seven-fold, eight-fold, nine-fold, or ten-fold in the media of the cell culture. In one embodiment, the levels of an amino acid are reduced below the limit of detection in the media of the cell culture.

In some embodiments of the above described genetically engineered bacteria, the gene encoding an amino acid catabolism enzyme is present on a plasmid in the bacterium and operatively linked on the plasmid to the promoter that is induced under low-oxygen or anaerobic conditions. In other embodiments, the gene encoding an amino acid catabolism enzyme is present in the bacterial chromosome and is operatively linked in the chromosome to the promoter that is induced under low-oxygen or anaerobic conditions.

In some embodiments, the genetically engineered bacteria comprising an amino acid catabolism enzyme is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyA and ΔdapA auxotroph.

In some embodiments, the genetically engineered bacteria comprising an amino acid catabolism enzyme further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of an promoter having a TetR repressor binding site and a gene encoding the TetR under the control of an inducible promoter that is induced by arabinose, such as P_{araBAD}. In some embodiments, the genetically engineered bacteria further comprise one or more genes encoding an antitoxin.

In some embodiments, the genetically engineered bacteria is an auxotroph comprising an amino acid catabolism enzyme gene and further comprises a kill-switch circuit, such as any of the kill-switch circuits described herein.

In some embodiments of the above described genetically engineered bacteria, the gene encoding an amino acid catabolism enzyme is present on a plasmid in the bacterium and operatively linked on the plasmid to the promoter that is induced under low-oxygen or anaerobic conditions. In other embodiments, the gene encoding an amino acid catabolism enzyme is present in the bacterial chromosome and is operatively linked in the chromosome to the promoter that is induced under low-oxygen or anaerobic conditions.

Amino Acid Catabolism Enzymes

As used herein, the term “amino acid catabolism enzyme” refers to an enzyme involved in the processing, degradation, or breakdown of an amino acid to a non-toxic molecule or other non-toxic byproducts. Enzymes involved in the catabolism of amino acids may be expressed or modified in the bacteria disclosed herein in order to enhance catabolism of at least one amino acid. Specifically, when at least one amino acid catabolism enzyme is expressed in the recombinant bacterial cells disclosed herein, the bacterial cells convert more of the target amino acid into one or more byproducts when the catabolism enzyme is expressed than unmodified bacteria of the same bacterial subtype under the same conditions. Thus, the genetically engineered bacteria comprising a heterologous gene encoding at least one amino acid catabolism enzyme can catabolize the target amino acid to treat a disease and/or disorder, e.g., cancer.

In one embodiment, the amino acid catabolism enzyme catabolizes leucine. In another embodiment, the amino acid catabolism enzyme catabolizes isoleucine. In another embodiment, the amino acid catabolism enzyme catabolizes valine. In another embodiment, the amino acid catabolism enzyme catabolizes arginine. In another embodiment, the amino acid catabolism enzyme catabolizes lysine. In another embodiment, the amino acid catabolism enzyme catabolizes asparagine. In another embodiment, the amino acid catabolism enzyme catabolizes serine. In another embodiment, the amino acid catabolism enzyme catabolizes glutamate. In another embodiment, the amino acid catabolism enzyme catabolizes tryptophan. In another embodiment, the amino acid catabolism enzyme catabolizes methionine. In another embodiment, the amino acid catabolism enzyme catabolizes threonine. In another embodiment, the amino acid catabolism enzyme catabolizes cysteine. In another embodiment, the amino acid catabolism enzyme catabolizes tyrosine. In another embodiment, the amino acid catabolism enzyme catabolizes phenylalanine. In another embodiment, the amino acid catabolism enzyme catabolizes glutamic acid. In another embodiment, the amino acid catabolism enzyme catabolizes histidine. In another embodiment, the amino acid catabolism enzyme catabolizes proline.

In one embodiment, the amino acid catabolism enzyme increases the rate of catabolism of at least one amino acid in the cell. In one embodiment, the amino acid catabolism enzyme decreases the level of at least one amino acid in the cell or in the subject. In another embodiment, the amino acid catabolism enzyme increases the level of an amino acid byproduct in the cell or in the subject as compared to the level of the catabolized amino acid in the cell or in the subject.

In one embodiment, the recombinant bacterial cell comprises a heterologous gene encoding an amino acid catabolism enzyme. In some embodiments, the disclosure provides a bacterial cell that comprises a heterologous gene encoding an amino acid catabolism enzyme operably linked to a first promoter, e.g., an inducible promoter or a constitutive promoter. In one embodiment, the bacterial cell comprises gene encoding an amino acid catabolism enzyme from a different organism, e.g., a different species of bacteria. In another embodiment, the bacterial cell comprises more than one copy of a native gene encoding an amino acid catabolism enzyme. In yet another embodiment, the bacterial cell comprises a native gene encoding an amino acid catabolism enzyme, as well as at least one copy of a gene encoding an amino acid catabolism enzyme from a different organism, e.g., a different species of bacteria. In one embodiment, the bacterial cell comprises at least one, two, three, four, five, or six copies of a gene encoding an amino acid catabolism enzyme. In one embodiment, the bacterial cell comprises multiple copies of a gene encoding an amino acid catabolism enzyme.

In one embodiment, the recombinant bacterial cell comprises a heterologous gene encoding an amino acid catabolism enzyme, wherein said amino acid catabolism enzyme comprises an amino acid sequence that has at least 80%, 81%, 82%, 83% 84%, 85%, 86%, 87%, 88%, 89%, 90%, 91%, 92%, 93%, 94%, 95%, 96%, 97%, 98%, 99%, or 100% identity to the amino acid sequence of a polypeptide encoded by an amino acid catabolism enzyme gene disclosed herein.

Multiple distinct an amino acid catabolism enzymes are known in the art. In some embodiments, amino acid catabolism enzyme is encoded by a gene encoding an amino acid catabolism enzyme derived from a bacterial species. In some embodiments, an amino acid catabolism enzyme is encoded by a gene encoding an amino acid catabolism enzyme derived from a non-bacterial species. In some embodiments, an amino acid catabolism enzyme is encoded by a gene derived from a eukaryotic species, e.g., protozoan species, a fungal species, a yeast species, or a plant species. In one embodiment, an amino acid catabolism enzyme is encoded by a gene derived from a human. In one embodiment, the gene encoding the amino acid catabolism enzyme is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Pseudomonas, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartite, Nasonia vitipennis, Nostoc punctiforme, Pantoea ananatis, Pantoea agglomerans, Pectobacterium atrosepticum, Pectobacterium carotovorum, Pseudomonas aeruginosa, Psychrobacter anticus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae.

In one embodiment, the at least one gene encoding the at least one amino acid catabolism enzyme is derived from an organism of the genus or species that includes, but is not limited to, Achromobacter parvulus, Acidomonas methanolica, Agrobacterium tumefaciens, Aminobacter aminovorans, Ancylobacter aquaticus, Arthrobacter spp., Bacillus spp., such as Bacillus amyloliquefaciens, Bacillus atrophaeus, Bacillus methanolicus, Bacillus halodurans, or Bacillus subtilis, Beggiatoa alba, Ceriporiopsis subvermispora, Clostridium botulinum, Clostridium carboxidivorans, Corynebacterium glutamicum, Cupriavidus necator, Cupriavidus oxalaticus, Desulfovibrio desulfuricans, Escherichia coli, Flavobacterium spp., such as Flavobacterium limnosediminis, Glycine max, Glycine soja, Gottschalkia acidurici, Helicobacter pylori, Hyphomicrobium spp., Klebsiella spp., such as Klebsiella pneumoniae or Klebsiella quasipneumoniae, Kloeckera spp., Komagataella pastrois, Lactobacillus spp., such as Lactobacillus saniviri, Lotus japonicas, Methylobacterium spp., such as Methylobacterium aquaticum, Methylobacterium extorquens, Methylobacterium organophilum, Methylobacterium lusitanum, Methylobacterium oryzae, or Methylobacterium salsuginis, Methylococcus spp., such as Methylococcus capsulatus, Methylomicrobium album, or Methylophaga spp., Methylocella silvestris, Methylophaga spp., such as Methylophaga marina or Methylophaga thalassica, Methylophilus methylotrophus, Methylosinus trichosporium, Methyloversatilis universalis, Methylovorus mays, Moraxella spp., Mycobacterium spp., such as Mycobacterium bovis or Mycobacterium vaccae, Ogataea angusta, Ogataea pini, Paracoccus spp., such as Paracoccus dentrificans, Pisum sativum, Pseudomonas spp., such as Pseudomonas putida or Pseudomonas methylica or Pseudomonas fluorescens, Rastrelliger kanagurta, Rhodopseudomonas palustris, Salmonella spp., such as Salmonella enterica, Sinorhizobium meliloti, Thiobacillus spp., or Viqna radiate. In another embodiment, the at least one gene encoding the at least one amino acid catabolism enzyme is derived from an organism of the genus or species that includes, but is not limited to Arabidopsis thaliana, Candida spp., such as Candida boidinii, Candida methanolica, or Candida methylica, Saccharomyces cerevisiae, or Torulopsis candida.

In one embodiment, the at least one gene encoding the at least one amino acid catabolism enzyme is derived from an organism of the genus or species that includes, but is not limited to, Bifidobacterium, Bordetella, Bradyrhizobium, Burkholderia, Clostridium, Enterococcus, Escherichia, Eubacterium, Lactobacillus, Magnetospirillium, Mycobacterium, Neurospora, Oxalobacter, Ralstonia, Rhodopseudomonas, Shigella, Thermoplasma, and Thauera, e.g., Bifidobacterium animalis, Bifidobacterium bifidum, Bifidobacterium infantis, Bifidobacterium lactis, Bifidobacterium longum, Bordatella bronchiseptica, Bordatella parapertussis, Burkholderia fungorum, Burkholderia xenovorans, Bradyrhizobium japonicum, Clostridium acetobutylicum, Clostridium difficile, Clostridium scindens, Clostridium sporogenes, Clostridium tentani, Enterococcus faecalis, Escherichia coli, Eubacterium lentum, Lactobacillus acidophilus, Lactobacillus bulgaricus, Lactobacillus casei, Lactobacillus gasseri, Lactobacillus plantarum, Lactobacillus rhamnosus, Lactococcus lactis, Magnetospirillium magentotaticum, Mycobacterium avium, Mycobacterium intracellulare, Mycobacterium kansasii, Mycobacterium leprae, Mycobacterium smegmatis, Mycobacterium tuberculosis, Mycobacterium ulcerans, Neurospora crassa, Oxalobacter formigenes, Ralstonia eutropha, Ralstonia metallidurans, Rhodopseudomonas palustris, Shigella flexneri, Thermoplasma volcanium, and Thauera aromatics.

In one embodiment, the at least one gene encoding the at least one amino acid catabolism enzyme has been codon-optimized for use in the recombinant bacterial cell disclosed herein. In one embodiment, the at least one gene encoding the at least one amino acid catabolism enzyme has been codon-optimized for use in Escherichia coli. In another embodiment, the at least one gene encoding the at least one amino acid catabolism enzyme has been codon-optimized for use in Lactococcus.

When the at least one gene encoding the at least one amino acid catabolism enzyme is expressed in the recombinant bacterial cells disclosed herein, the bacterial cells catabolize more of the target amino acid than unmodified bacteria of the same bacterial subtype under the same conditions (e.g., culture or environmental conditions). Thus, the genetically engineered bacteria comprising at least one heterologous gene encoding at least one amino acid catabolism enzyme may be used to catabolize any amino acid of interest in order to treat a disease and/or disorder associated with amino acid metabolism, e.g., cancer.

The present disclosure further provides genes encoding functional fragments of at least one amino acid catabolism enzyme or functional variants of at least one amino acid catabolism enzyme. As used herein, the term “functional fragment thereof” or “functional variant thereof” of at least one amino acid catabolism enzyme relates to an element having qualitative biological activity in common with the wild-type amino acid catabolism enzyme from which the fragment or variant was derived (e.g., a domain of the amino acid catabolism enzyme). For example, a functional fragment or a functional variant of a mutated amino acid catabolism enzyme is one which retains essentially the same ability to catabolize amino acids as the amino acid catabolism enzyme from which the functional fragment or functional variant was derived. For example a polypeptide having amino acid catabolism enzyme activity may be truncated at the N-terminus or C-terminus and the retention of amino acid catabolism enzyme activity assessed using assays known to those of skill in the art, including the exemplary assays provided herein. In one embodiment, the recombinant bacterial cell disclosed herein comprises a heterologous gene encoding at least one amino acid catabolism enzyme functional variant. In another embodiment, the recombinant bacterial cell disclosed herein comprises a heterologous gene encoding at least one amino acid catabolism enzyme functional fragment.

In some embodiments, the gene encoding an amino acid catabolism enzyme is mutagenized; mutants exhibiting increased activity are selected; and the mutagenized gene encoding the amino acid catabolism enzyme is isolated and inserted into the bacterial cell described herein. In one embodiment, spontaneous mutants that arise that allow bacteria to grow on amino acids as the sole carbon source can be screened for and selected. The gene comprising the modifications described herein may be present on a plasmid or chromosome.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The present disclosure encompasses genes encoding at least one amino acid catabolism enzyme comprising amino acids in its sequence that are substantially the same as an amino acid sequence described herein Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

Assays for testing the activity of an amino acid catabolism enzyme, an amino acid catabolism enzyme functional variant, or an amino acid catabolism enzyme functional fragment are well known to one of ordinary skill in the art. For example, amino acid catabolism can be assessed by expressing the protein, functional variant, or fragment thereof, in a recombinant bacterial cell that lacks endogenous amino acid catabolism enzyme activity. Amino acid catabolism can be assessed using the coupled enzymatic assay method as described by Zhang et al. (see, for example, Zhang et al., Proc. Natl. Acad. Sci., 105(52):20653-58, 2008). Furthermore, catabolism of amino acids can also be assessed in vitro by measuring the disappearance of amino acids as described by de la Plaza (see, for example, de la Plaza et al., FEMS Microbiol. Letters, 2004, 238(2):367-374). Additional assays are described in detail in the amino acid catabolism enzyme subsections, below.

In one embodiment, the bacterial cell disclosed herein comprises at least one heterologous gene encoding at least one amino acid catabolism enzyme. In one embodiment, the recombinant bacterial cells described herein comprise one amino acid catabolism enzyme. In another embodiment, the recombinant bacterial cells described herein comprise two amino acid catabolism enzymes. In another embodiment, the recombinant bacterial cells described herein comprise three amino acid catabolism enzymes. In another embodiment, the recombinant bacterial cells described herein comprise four amino acid catabolism enzymes. In another embodiment, the recombinant bacterial cells described herein comprise five amino acid catabolism enzymes.

In some embodiments, the disclosure provides a bacterial cell that comprises at least one heterologous gene encoding at least one amino acid catabolism enzyme operably linked to a first promoter. In one embodiment, the first promoter is an inducible promoter. In one embodiment, the first promoter is a constitutive promoter. In one embodiment, the bacterial cell comprises at least one gene encoding at least one amino acid catabolism enzyme from a different organism, e.g., a different species of bacteria. In another embodiment, the bacterial cell comprises more than one copy of a native gene encoding at least one amino acid catabolism enzyme. In yet another embodiment, the bacterial cell comprises at least one native gene encoding at least one amino acid catabolism enzyme, as well as at least one copy of at least one gene encoding at least one amino acid catabolism enzyme from a different organism, e.g., a different species of bacteria. In one embodiment, the bacterial cell comprises at least one, two, three, four, five, or six copies of a gene encoding at least one amino acid catabolism enzyme. In one embodiment, the bacterial cell comprises multiple copies of a gene or genes encoding at least one amino acid catabolism enzyme. In one embodiment, the gene encoding the amino acid catabolism enzyme is directly operably linked to a first promoter. In another embodiment, the gene encoding the amino acid catabolism enzyme is indirectly operably linked to a first promoter. In one embodiment, the gene encoding the amino acid catabolism enzyme is operably linked to a promoter that is not associated with the amino acid catabolism gene in nature.

In some embodiments, the gene encoding the amino acid catabolism enzyme is expressed under the control of a constitutive promoter. In another embodiment, the gene encoding the amino acid catabolism enzyme is expressed under the control of an inducible promoter. In some embodiments, the gene encoding the amino acid catabolism enzyme is expressed under the control of a promoter that is directly or indirectly induced by exogenous environmental conditions. In one embodiment, the gene encoding the amino acid catabolism enzyme is expressed under the control of a promoter that is directly or indirectly induced by low-oxygen or anaerobic conditions, wherein expression of the gene encoding the amino acid catabolism enzyme is activated under low-oxygen or anaerobic environments, such as the environment of the mammalian gut. Inducible promoters are described in more detail infra.

The gene encoding the amino acid catabolism enzyme may be present on a plasmid or chromosome in the bacterial cell. In one embodiment, the gene encoding the amino acid catabolism enzyme is located on a plasmid in the bacterial cell. In another embodiment, the gene encoding the amino acid catabolism enzyme is located in the chromosome of the bacterial cell. In yet another embodiment, a native copy of the gene encoding the amino acid catabolism enzyme is located in the chromosome of the bacterial cell, and a gene encoding an amino acid catabolism enzyme from a different species of bacteria is located on a plasmid in the bacterial cell. In yet another embodiment, a native copy of the gene encoding the amino acid catabolism enzyme is located on a plasmid in the bacterial cell, and a gene encoding the amino acid catabolism enzyme from a different species of bacteria is located on a plasmid in the bacterial cell. In yet another embodiment, a native copy of the gene encoding the amino acid catabolism enzyme is located in the chromosome of the bacterial cell, and a gene encoding the amino acid catabolism enzyme from a different species of bacteria is located in the chromosome of the bacterial cell.

In some embodiments, the gene encoding the amino acid catabolism enzyme is expressed on a low-copy plasmid. In some embodiments, the gene encoding the amino acid catabolism enzyme is expressed on a high-copy plasmid. In some embodiments, the high-copy plasmid may be useful for increasing expression of the amino acid catabolism enzyme, thereby increasing the catabolism of the amino acid.

In some embodiments, a recombinant bacterial cell comprising the gene encoding the amino acid catabolism enzyme expressed on a high-copy plasmid does not increase amino acid catabolism or decrease amino acid levels as compared to a recombinant bacterial cell comprising the same gene expressed on a low-copy plasmid in the absence of a heterologous transporter of the amino acid and additional copies of a native transporter of the amino acid. It has been surprisingly discovered that in some embodiments, the rate-limiting step of amino acid catabolism is not expression of an amino acid catabolism enzyme, but rather availability of the amino acid. Thus, in some embodiments, it may be advantageous to increase amino acid transport into the cell, thereby enhancing amino acid catabolism. The inventors of the instant application have surprisingly found that, in conjunction with overexpression of a transporter of an amino acid even low copy number plasmids comprising a gene encoding an amino acid catabolism enzyme are capable of almost completely eliminating an amino acid from a sample. Furthermore, in some embodiments that incorporate a transporter of an amino acid into the recombinant bacterial cell, there may be additional advantages to using a low-copy plasmid comprising the gene encoding the amino acid catabolism enzyme in conjunction in order to enhance the stability of expression of the amino acid catabolism enzyme, while maintaining high amino acid catabolism and to reduce negative selection pressure on the transformed bacterium. In alternate embodiments, the amino acid transporter is used in conjunction with a high-copy plasmid.

In one embodiment, the amino acid catabolism enzyme catabolizes arginine. In another embodiment, the amino acid catabolism enzyme catabolizes asparagine. In another embodiment, the amino acid catabolism enzyme catabolizes serine. In another embodiment, the amino acid catabolism enzyme catabolizes glycine. In another embodiment, the amino acid catabolism enzyme catabolizes tryptophan. In another embodiment, the amino acid catabolism enzyme catabolizes methionine. In another embodiment, the amino acid catabolism enzyme catabolizes threonine. In another embodiment, the amino acid catabolism enzyme catabolizes cysteine. In another embodiment, the amino acid catabolism enzyme catabolizes tyrosine. In another embodiment, the amino acid catabolism enzyme catabolizes phenylalanine. In another embodiment, the amino acid catabolism enzyme catabolizes glutamic acid. In another embodiment, the amino acid catabolism enzyme catabolizes histidine. In another embodiment, the amino acid catabolism enzyme catabolizes proline.

Multiple distinct amino acid catabolism enzymes are well known in the art and are described in the subsections, below.

Transporters of Amino Acids

See PCT/US2016/032565, filed May 13, 2016, which application is hereby incorporated by reference in its entirety, including the drawings. The uptake of amino acids into bacterial cells is mediated by proteins well known to those of skill in the art Amino acid transporters, e.g., amino acid transporters, may be expressed or modified in the bacteria in order to enhance amino acid transport into the cell. Specifically, when the transporter of an amino acid is expressed in the recombinant bacterial cells, the bacterial cells import more amino acid(s) into the cell when the transporter is expressed than unmodified bacteria of the same bacterial subtype under the same conditions. Thus, the genetically engineered bacteria comprising a heterologous gene encoding a transporter of an amino acid, which may be used to import an amino acid(s) into the bacteria so that any gene encoding an amino acid catabolism enzyme expressed in the organism, e.g., co-expressed amino acid catabolism enzyme, can catabolize the amino acid to treat diseases associated with the catabolism of amino acids, such as cancer. In one embodiment, the bacterial cell comprises a heterologous gene encoding one or more transporter(s) of an amino acid. In one embodiment, the bacterial cell comprises a heterologous gene encoding a transporter of an amino acid and a heterologous gene encoding one or more amino acid catabolism enzymes. In one embodiment, the bacterial cell comprises a heterologous gene encoding a transporter of amino acid and a genetic modification that reduces export of an amino acid, e.g., a genetic mutation in an exporter gene or promoter. In one embodiment, the bacterial cell comprises a heterologous gene encoding a transporter of an amino acid, a heterologous gene encoding an amino acid catabolism enzyme, and a genetic modification that reduces export of an amino acid.

Thus, in some embodiments, disclosed herein is a bacterial cell that comprises a heterologous gene encoding an amino acid catabolism enzyme operably linked to a first promoter and at least one heterologous gene encoding a transporter of an amino acid. In some embodiments, disclosed herein is a bacterial cell that comprises at least one heterologous gene encoding a transporter of an amino acid operably linked to the first promoter. In another embodiment, disclosed herein is a bacterial cell that comprises a heterologous gene encoding an amino acid catabolism enzyme operably linked to a first promoter and at least one heterologous gene encoding a transporter of an amino acid operably linked to a second promoter. In one embodiment, the first promoter and the second promoter are separate copies of the same promoter. In another embodiment, the first promoter and the second promoter are different promoters.

In one embodiment, the bacterial cell comprises at least one gene encoding a transporter of an amino acid from a different organism, e.g., a different species of bacteria. In one embodiment, the bacterial cell comprises at least one native gene encoding a transporter of an amino acid. In some embodiments, the at least one native gene encoding a transporter of an amino acid is not modified. In another embodiment, the bacterial cell comprises more than one copy of at least one native gene encoding a transporter of an amino acid. In yet another embodiment, the bacterial cell comprises a copy of at least one gene encoding a native transporter of an amino acid, as well as at least one copy of at least one heterologous gene encoding a transporter of an amino acid from a different bacterial species. In one embodiment, the bacterial cell comprises at least one, two, three, four, five, or six copies of the at least one heterologous gene encoding a transporter of an amino acid. In one embodiment, the bacterial cell comprises multiple copies of the at least one heterologous gene encoding a transporter of an amino acid.

In one embodiment, the recombinant bacterial cell comprises a heterologous gene encoding an amino acid transporter (e.g., an amino acid transporter), wherein said amino acid transporter comprises an amino acid sequence that has at least 80%, 81%, 82%, 83% 84%, 85%, 86%, 87%, 88%, 89%, 90%, 91%, 92%, 93%, 94%, 95%, 96%, 97%, 98%, 99%, or 100% identity to the amino acid sequence of a polypeptide encoded by an amino acid transporter gene disclosed herein.

In some embodiments, the transporter of an amino acid is encoded by a transporter of an amino acid gene derived from a bacterial genus or species, including but not limited to, Bacillus, Campylobacter, Clostridium, Escherichia, Lactobacillus, Pseudomonas, Salmonella, Staphylococcus, Bacillus subtilis, Campylobacter jejuni, Clostridium perfringens, Escherichia coli, Lactobacillus delbrueckii, Pseudomonas aeruginosa, Salmonella typhimurium, or Staphylococcus aureus. In some embodiments, the bacterial species is Escherichia coli. In some embodiments, the bacterial species is Escherichia coli strain Nissle.

The present disclosure further comprises genes encoding functional fragments of a transporter of an amino acid or functional variants of a transporter of an amino acid. As used herein, the term “functional fragment thereof” or “functional variant thereof” of a transporter of an amino acid relates to an element having qualitative biological activity in common with the wild-type transporter of an amino acid from which the fragment or variant was derived. For example, a functional fragment or a functional variant of a mutated transporter of an amino acid protein is one which retains essentially the same ability to import leucine into the bacterial cell as does the transporter protein from which the functional fragment or functional variant was derived. In one embodiment, the recombinant bacterial cell comprises at least one heterologous gene encoding a functional fragment of a transporter of amino acid. In another embodiment, the recombinant bacterial cell comprises a heterologous gene encoding a functional variant of a transporter of amino acid.

Assays for testing the activity of a transporter of an amino acid, a functional variant of a transporter of an amino acid, or a functional fragment of transporter of an amino acid are well known to one of ordinary skill in the art. For example, import of an amino acid may be determined using the methods as described in Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of each of which are expressly incorporated by reference herein.

In one embodiment, the genes encoding the transporter of an amino acid have been codon-optimized for use in the host organism. In one embodiment, the genes encoding the transporter of an amino acid have been codon-optimized for use in Escherichia coli.

The present disclosure also encompasses genes encoding a transporter of an amino acid comprising amino acids in its sequence that are substantially the same as an amino acid sequence described herein Amino acid sequences that are substantially the same as the sequences described herein include sequences comprising conservative amino acid substitutions, as well as amino acid deletions and/or insertions.

In some embodiments, the at least one gene encoding a transporter of an amino acid is mutagenized; mutants exhibiting increased amino acid transport are selected; and the mutagenized at least one gene encoding a transporter of an amino acid is isolated and inserted into the bacterial cell. In some embodiments, the at least one gene encoding a transporter of an amino acid is mutagenized; mutants exhibiting decreased amino acid transport are selected; and the mutagenized at least one gene encoding a transporter of an amino acid is isolated and inserted into the bacterial cell. The transporter modifications described herein may be present on a plasmid or chromosome.

In some embodiments, the bacterial cell comprises a heterologous gene encoding an amino acid catabolism enzyme operably linked to a first promoter and at least one heterologous gene encoding a transporter of an amino acid. In some embodiments, the at least one heterologous gene encoding a transporter of an amino acid is operably linked to the first promoter. In other embodiments, the at least one heterologous gene encoding a transporter of an amino acid is operably linked to a second promoter. In one embodiment, the at least one gene encoding a transporter of an amino acid is directly operably linked to the second promoter. In another embodiment, the at least one gene encoding a transporter of an amino acid is indirectly operably linked to the second promoter.

In some embodiments, expression of at least one gene encoding a transporter of an amino acid is controlled by a different promoter than the promoter that controls expression of the gene encoding the amino acid catabolism enzyme. In some embodiments, expression of the at least one gene encoding a transporter of an amino acid is controlled by the same promoter that controls expression of the amino acid catabolism enzyme. In some embodiments, at least one gene encoding a transporter of an amino acid and the amino acid catabolism enzyme are divergently transcribed from a promoter region. In some embodiments, expression of each of genes encoding the at least one gene encoding a transporter of an amino acid and the gene encoding the amino acid catabolism enzyme is controlled by different promoters.

In one embodiment, the promoter is not operably linked with the at least one gene encoding a transporter of an amino acid in nature. In some embodiments, the at least one gene encoding the transporter of an amino acid is controlled by its native promoter. In some embodiments, the at least one gene encoding the transporter of an amino acid is controlled by an inducible promoter. In some embodiments, the at least one gene encoding the transporter of an amino acid is controlled by a promoter that is stronger than its native promoter. In some embodiments, the at least one gene encoding the transporter of an amino acid is controlled by a constitutive promoter.

In another embodiment, the promoter is an inducible promoter. Inducible promoters are described in more detail infra.

In one embodiment, the at least one gene encoding a transporter of an amino acid is located on a plasmid in the bacterial cell. In another embodiment, the at least one gene encoding a transporter of an amino acid is located in the chromosome of the bacterial cell. In yet another embodiment, a native copy of the at least one gene encoding a transporter of an amino acid is located in the chromosome of the bacterial cell, and a copy of at least one gene encoding a transporter of an amino acid from a different species of bacteria is located on a plasmid in the bacterial cell. In yet another embodiment, a native copy of the at least one gene encoding a transporter of an amino acid is located on a plasmid in the bacterial cell, and a copy of at least one gene encoding a transporter of an amino acid from a different species of bacteria is located on a plasmid in the bacterial cell. In yet another embodiment, a native copy of the at least one gene encoding a transporter of an amino acid is located in the chromosome of the bacterial cell, and a copy of the at least one gene encoding a transporter of an amino acid from a different species of bacteria is located in the chromosome of the bacterial cell.

In some embodiments, the at least one native gene encoding the transporter in the bacterial cell is not modified, and one or more additional copies of the native transporter are inserted into the genome. In one embodiment, the one or more additional copies of the native transporter that is inserted into the genome are under the control of the same inducible promoter that controls expression of the gene encoding the amino acid catabolism enzyme, e.g., the FNR promoter, or a different inducible promoter than the one that controls expression of the amino acid catabolism enzyme, or a constitutive promoter. In alternate embodiments, the at least one native gene encoding the transporter is not modified, and one or more additional copies of the transporter from a different bacterial species is inserted into the genome of the bacterial cell. In one embodiment, the one or more additional copies of the transporter inserted into the genome of the bacterial cell are under the control of the same inducible promoter that controls expression of the gene encoding the amino acid catabolism enzyme, e.g., the FNR promoter, or a different inducible promoter than the one that controls expression of the gene encoding the amino acid catabolism enzyme, or a constitutive promoter.

In some embodiments, at least one native gene encoding the transporter in the genetically modified bacteria is not modified, and one or more additional copies of at least one native gene encoding the transporter are present in the bacterial cell on a plasmid. In one embodiment, the at least one native gene encoding the transporter present in the bacterial cell on a plasmid is under the control of the same inducible promoter that controls expression of the gene encoding the amino acid catabolism enzyme, e.g., the FNR promoter, or a different inducible promoter than the one that controls expression of the gene encoding the amino acid catabolism enzyme, or a constitutive promoter. In alternate embodiments, the at least one native gene encoding the transporter is not modified, and a copy of at least one gene encoding the transporter from a different bacterial species is present in the bacteria on a plasmid. In one embodiment, the copy of at least one gene encoding the transporter from a different bacterial species is under the control of the same inducible promoter that controls expression of the gene encoding the amino acid catabolism enzyme, e.g., the FNR promoter, or a different inducible promoter than the one that controls expression of the gene encoding the amino acid catabolism enzyme, or a constitutive promoter.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome under the control of the same inducible promoter that controls expression of the gene encoding the amino acid catabolism enzyme, e.g., the FNR promoter, or a different inducible promoter than the one that controls expression of the gene encoding the amino acid catabolism enzyme, or a constitutive promoter. In an alternate embodiment, the at least one native gene encoding the transporter in E. coli Nissle is not modified, and a copy of at least one gene encoding the transporter from a different bacterial species is inserted into the E. coli Nissle genome under the control of the same inducible promoter that controls expression of the gene encoding the amino acid catabolism enzyme, e.g., the FNR promoter, or a different inducible promoter than the one that controls expression of the gene encoding the amino acid catabolism enzyme, or a constitutive promoter.

In one embodiment, when the transporter of an amino acid is expressed in the recombinant bacterial cells, the bacterial cells import 10% more amino acids into the bacterial cell when the transporter is expressed than unmodified bacteria of the same bacterial subtype under the same conditions. In another embodiment, when the transporter of an amino acid is expressed in the recombinant bacterial cells, the bacterial cells import 20%, 30%, 40%, 50%, 60%, 70%, 80%, 90% or 100% more amino acids, into the bacterial cell when the transporter is expressed than unmodified bacteria of the same bacterial subtype under the same conditions. In yet another embodiment, when the transporter of an amino acid is expressed in the recombinant bacterial cells, the bacterial cells import two-fold more amino acids into the cell when the transporter is expressed than unmodified bacteria of the same bacterial subtype under the same conditions. In yet another embodiment, when the transporter of an amino acid is expressed in the recombinant bacterial cells, the bacterial cells import three-fold, four-fold, five-fold, six-fold, seven-fold, eight-fold, nine-fold, or ten-fold more amino acids into the cell when the transporter is expressed than unmodified bacteria of the same bacterial subtype under the same conditions.

In one embodiment, the recombinant bacterial cells described herein further comprise at least one amino acid transporter. In another embodiment, the recombinant bacterial cells described herein comprise two amino acid transporters. In another embodiment, the recombinant bacterial cells described herein comprise three amino acid transporters. In another embodiment, the recombinant bacterial cells described herein comprise four amino acid transporters. In another embodiment, the recombinant bacterial cells described herein comprise five amino acid transporters.

In one embodiment, the transporter of an amino acid imports an amino acid into the bacterial cell. In one embodiment, the transporter of an amino acid is a transporter of arginine. In another embodiment, the transporter of an amino acid is a transporter of asparagine. In another embodiment, the transporter of an amino acid is a transporter of serine. In another embodiment, the transporter of an amino acid is a transporter of glycine. In another embodiment, the transporter of an amino acid is a transporter of tryptophan. In another embodiment, the transporter of an amino acid is a transporter of methionine. In another embodiment, the transporter of an amino acid is a transporter of threonine. In another embodiment, the transporter of an amino acid is a transporter of cysteine. In another embodiment, the transporter of an amino acid is a transporter of tyrosine. In another embodiment, the transporter of an amino acid is a transporter of phenylalanine. In another embodiment, the transporter of an amino acid is a transporter of glutamic acid. In another embodiment, the transporter of an amino acid is a transporter of histidine. In another embodiment, the transporter of an amino acid is a transporter of proline.

Multiple distinct transporters of amino acids are well known in the art and are described in the subsections, below.

Exporters of Amino Acids

The export of amino acids from bacterial cells is mediated by proteins well known to those of skill in the art. The bacterial cells may comprise a genetic modification that reduces export of an amino acid from the bacterial cell.

In one embodiment, the recombinant bacterial cell comprises a genetic modification that reduces export of an amino acid from the bacterial cell and a heterologous gene encoding an amino acid catabolism enzyme. When the recombinant bacterial cells comprise a genetic modification that reduces export of an amino acid, the bacterial cells retain more amino acids in the bacterial cell than unmodified bacteria of the same bacterial subtype under the same conditions. Thus, the recombinant bacteria comprising a genetic modification that reduces export of an amino acid may be used to retain more amino acids in the bacterial cell so that any amino acid catabolism enzyme expressed in the organism can catabolize the amino acids to treat diseases associated with the catabolism of amino acids, including cancer. In one embodiment, the recombinant bacteria further comprise a heterologous gene encoding a transporter of an amino acid gene.

In one embodiment, the recombinant bacterial cell comprises a genetic modification in a gene encoding an amino acid exporter, wherein said amino acid exporter comprises an amino acid sequence that has at least 80%, 81%, 82%, 83% 84%, 85%, 86%, 87%, 88%, 89%, 90%, 91%, 92%, 93%, 94%, 95%, 96%, 97%, 98%, 99%, or 100% identity to the amino acid sequence of a polypeptide encoded by an amino acid exporter gene disclosed herein.

In one embodiment, the genetic modification reduces export of an amino acid from the bacterial cell. In one embodiment, the bacterial cell is from a bacterial genus or species that includes but is not limited to, Bacillus, Bacteroides, Bifidobacterium, Brevibacteria, Clostridium, Enterococcus, Escherichia, Lactobacillus, Lactococcus, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis. In another embodiment, the bacterial cell is an Escherichia coli bacterial cell. In another embodiment, the bacterial cell is an Escherichia coli strain Nissle bacterial cell.

In one embodiment, the genetic modification is a mutation in an endogenous gene encoding an exporter of an amino acid. In one embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity, i.e., results in an exporter which cannot export an amino acid from the bacterial cell.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of the exporter of an amino acid. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254(2):157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, for example, Datta et al., Gene, 379:109-115 (2006)).

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

Assays for testing the activity of an exporter of an amino acid are well known to one of ordinary skill in the art. For example, export of an amino acid may be determined using the methods described by Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of which are expressly incorporated herein by reference.

In another embodiment, the genetic modification is a mutation in a promoter of an endogenous gene encoding an exporter of an amino acid. In one embodiment, the genetic mutation results in decreased expression of the exporter gene. In one embodiment, exporter gene expression is reduced by about 50%, 75%, or 100%. In another embodiment, exporter gene expression is reduced about two-fold, three-fold, four-fold, or five-fold. In another embodiment, the genetic mutation completely inhibits expression of the exporter gene.

Assays for testing the level of expression of a gene, such as an exporter of an amino acid are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In another embodiment, the genetic modification is an overexpression of a repressor of an exporter of an amino acid. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

In one embodiment, the recombinant bacterial cells described herein comprise at least one genetic modification that reduces export of an amino acid from the bacterial cell. In another embodiment, the recombinant bacterial cells described herein comprise two genetic modifications that reduce export of an amino acid from the bacterial cell. In another embodiment, the recombinant bacterial cells described herein comprise three genetic modifications that reduce export of an amino acid from the bacterial cell. In another embodiment, the recombinant bacterial cells described herein comprise four genetic modifications that reduce export of an amino acid from the bacterial cell. In another embodiment, the recombinant bacterial cells described herein comprise five genetic modifications that reduce export of an amino acid from the bacterial cell.

In one embodiment, the exporter of an amino acid exports an amino acid out of the bacterial cell. In one embodiment, the exporter of an amino acid is an exporter of arginine. In another embodiment, the exporter of an amino acid is an exporter of asparagine. In another embodiment, the exporter of an amino acid is an exporter of serine. In another embodiment, the exporter of an amino acid is an exporter of glycine. In another embodiment, the exporter of an amino acid is an exporter of tryptophan. In another embodiment, the exporter of an amino acid is an exporter of methionine. In another embodiment, the exporter of an amino acid is an exporter of threonine. In another embodiment, the exporter of an amino acid is an exporter of cysteine. In another embodiment, the exporter of an amino acid is an exporter of tyrosine. In another embodiment, the exporter of an amino acid is an exporter of phenylalanine. In another embodiment, the exporter of an amino acid is an exporter of glutamic acid. In another embodiment, the exporter of an amino acid is an exporter of histidine. In another embodiment, the exporter of an amino acid is an exporter of proline.

Multiple distinct exporter of amino acids are well known in the art and are described in the subsections, below.

Specific Amino Acid Catabolism Enzymes, Transporters, and Exporters

Amino acid catabolism enzymes are described in more detail in the subsections, below.

1. Branched Chain Amino Acids: Leucine, Isoleucine, and Valine

The term “branched chain amino acid” or “BCAA,” as used herein, refers to an amino acid which comprises a branched side chain. Leucine, isoleucine, and valine are naturally occurring amino acids comprising a branched side chain. However, non-naturally occurring, usual, and/or modified amino acids comprising a branched side chain are also encompassed by the term branched chain amino acid.

The term “alpha-keto acid” or “α-keto acid” refers to the immediate precursor of a branched chain amino acid. α-ketoisocaproic acid (MC), α-ketoisovaleric acid (KIV), and α-keto-beta-methylvaleric acid (KMV) are naturally occurring alpha-keto acids. However, non-naturally occurring, unusual, or modified alpha-keto acids are also encompassed by the term “alpha-keto acid.” Conversion of a branched chain amino acid to its corresponding alpha-keto acid is the first step in branched chain amino acid catabolism and is reversible.

Genetic Circuits, Bacterial Strains, and gene sequences for catabolizing branched chain amino acids, e.g., leucine, isovaline, and valine are described in PCT/US2016/37098 filed Jun. 10, 2016 and U.S. Ser. No. 15/379,445 filed Dec. 14, 2016, both of which applications are hereby incorporated by reference herein in their entireties, including the drawings.

A. Branched Chain Amino Acid Catabolism Enzymes

Branched chain amino acid catabolism enzymes may be expressed or modified in the bacteria disclosed herein in order to enhance catabolism of one or more branched chain amino acids. As used herein, the term “branched chain amino acid catabolism enzyme” refers to an enzyme involved in the catabolism of a branched chain amino acid or its branched chain α-keto acid counterpart or its acyl-CoA counterpart. Specifically, when a branched chain amino acid catabolism enzyme is expressed in a recombinant bacterial cell, the bacterial cell hydrolyzes more branched chain amino acids into its branched chain alpha-keto acid counterpart or its acyl-CoA counterpart when the catabolism enzyme is expressed than unmodified bacteria of the same bacterial subtype under the same conditions. Thus, the genetically engineered bacteria comprising a heterologous gene encoding a branched chain amino acid catabolism enzyme can catabolize one or more branched chain amino acids to treat a disease associated with a branched chain amino acid, such as cancer.

In one embodiment, the branched chain amino acid catabolism enzyme increases the rate of branched chain amino acid catabolism in the cell. In one embodiment, the branched chain amino acid catabolism enzyme decreases the level of branched chain amino acids in the cell. In another embodiment, the branched chain amino acid catabolism enzyme increases the level of branched chain α-keto acid counterparts or acyl-CoA counterparts.

Enzymes involved in the catabolism of branched chain amino acids are well known to those of skill in the art. For example, in bacteria, α-ketoisovalerate decarboxylase enzymes are capable of converting α-keto acids into aldehydes. Specifically, the α-ketoisovalerate decarboxylase enzyme KivD is capable of metabolizing leucine, isoleucine, and valine by converting ketoisovalerate to isovaleraldehyde, 2-methylbutyraldehyde, and isobutyraldehyde (see, for example, de la Plaza et al., FEMS Microbiol. Lett. 2004, 238(2):367-374). In bacteria, branched chain keto acid dehydrogenases (“BCKDs”) are enzyme complexes that oxidatively decarboxylate all three branched chain keto acids into their respective acyl-CoA derivatives (see, for example, Massey et al., Bacteriol Rev., 40(1):42-54, 1976). Moreover, in mammals, dehydrogenases specific for 2-ketoisovalerate (EC 1.2.4.4) and 2-keto-3-methylvalerate and 2-keto-isocaproate (EC 1.2.4.3) have been identified (see, for example, Massey et al., Bacteriol Rev., 40(1):42-54, 1976). Other examples of branched chain amino acid metabolic enzymes include, but are not limited to, leucine dehydrogenase (e.g., LeuDH), branched chain amino acid aminotransferase (e.g., IlvE), branched chain α-ketoacid dehydrogenase (e.g., KivD), L-Amino acid deaminase (e.g., L-AAD), alcohol dehydrogenase (e.g., Adh2, YqhD)), and aldehyde dehydrogenase (e.g., PadA), and any other enzymes that catabolizes BCAA.

In some embodiments, the present disclosure provides an engineered bacteria comprising gene sequence(s) encoding one or more branched chain amino acid catabolic enzyme(s). In some embodiments, the branched chain amino acid catabolism enzyme is used to convert a branched chain amino acid, e.g., leucine, valine, isoleucine, to its corresponding α-keto-acid. In some embodiments, wherein a branched chain amino acid catabolism enzyme is used to convert a branched chain amino acid, e.g., leucine, valine, isoleucine, to its corresponding α-keto-acid, the engineered bacteria further comprise a branched chain amino acid catabolism enzyme to convert an α-keto-acid to its corresponding aldehyde. In some embodiments, the engineered bacteria may further comprise an alcohol dehydrogenase enzyme in order to convert the branched chain amino acid-derived aldehyde (e.g., isovaleraldehyde, isobutyraldehyde, 2-methylbutyraldehyde) to its respective alcohol. In some embodiments, the engineered bacteria may further comprise an aldehyde dehydrogenase enzyme in order to convert the branched chain amino acid-derived aldehyde (e.g., isovaleraldehyde, isobutyraldehyde, 2-methylbutyraldehyde) to its respective carboxylic acid. Enzymes involved in the catabolism of branched chain amino acids are well known to those of skill in the art. For example, in bacteria, leucine dehydrogenase (LeuDH), branched achain amino acid transferase (IlvE), amino acid oxidase (also known as amino acid deaminase) (L-AAD), as well as other known enzymes can be used to convert a BCAA to its corresponding α-keto acid, e.g., ketoisocaproate (KIC), ketoisovalerate (MV), and ketomethylvalerate (KMV). Also, for example, in bacteria, α-ketoisovalerate decarboxylase (KivD) enzymes are capable of converting α-keto acids into aldehydes (e.g., isovaleraldehyde, isobutyraldehyde, 2-methylbutyraldehyde). Specifically, the α-ketoisovalerate decarboxylase enzyme KivD is capable of metabolizing valine by converting ketoisovalerate to isobutyraldehyde (see, for example, de la Plaza et al., FEMS Microbiol. Lett. 2004, 238(2):367-374), is capable of metabolizing leucine by converting ketoisocaproate (KIC) to isovaleraldehyde, and capable of metabolizing isoleucine by converting ketomethylvalerate (KMV) to 2-methylbutyraldehyde. In bacteria, branched chain keto acid dehydrogenases (“BCKDs”) are enzyme complexes that oxidatively decarboxylate all three branched chain keto acids into their respective acyl-CoA derivatives (see, for example, Massey et al., Bacteriol Rev., 40(1):42-54, 1976). Leucine dehydrogenases, branched chain amino acid transamination enzymes (EC 2.6.1.42), and L-amino acid deaminases (L-AAD), which oxidatively deaminate branched chain amino acids into their respective alpha-keto acid, are also known (Baker et al., Structure, 3(7):693-705, 1995; Peng et al., J. Bact., 139(2):339-45, 1979; and Kline et al., J. Bact., 130(2):951-3, 1977; Song et al., Scientific Reports, Nature, 5:12694; DOI: 10:1038/srep12694 (2015)).

Moreover, in mammals, dehydrogenases specific for 2-ketoisovalerate (EC 1.2.4.4) and 2-keto-3-methylvalerate and 2-keto-isocaproate (EC 1.2.4.3) have been identified (see, for example, Massey et al., Bacteriol Rev., 40(1):42-54, 1976). In addition, enzymes for converting aldehydes derived from BCAAs (e.g., isovaleraldehyde, isobutyraldehyde, 2-methylbutyraldehyde) to alcohols or carboxylic acids are known and available. For example, alcohol dehydrogenases (e.g., Adh2, YqhD) can convert isovaleraldehyde, isobutyraldehyde, 2-methylbutyraldehyde to isopentanol, isobutanol, and 2-methylbutanol, respectively. Aldehyde dehydrogenases (e.g., PadA) can convert isovaleraldehyde, isobutyraldehyde, 2-methylbutyraldehyde to isovalerate, isobutyrate, and 2-methylbutyrate, respectively.

In one embodiment, the branched chain amino acid catabolism enzyme increases the rate of branched chain amino acid catabolism in the cell. In one embodiment, the branched chain amino acid catabolism enzyme decreases the level of branched chain amino acid in the cell as compared to the level of its corresponding α-keto acid in the cell. In another embodiment, the branched chain amino acid catabolism enzyme increases the level of α-keto acid in the cell as compared to the level of its corresponding branched chain amino acid in the cell. In one embodiment, the branched chain amino acid catabolism enzyme decreases the level of the branched chain amino acid in the cell as compared to the level of its corresponding Acyl-CoA derivative in the cell. In one embodiment, the branched chain amino acid catabolism enzyme increases the level of the acyl-CoA derivative in the cell as compared to the level of the branched chain amino acid in the cell.

In one embodiment, the branched chain amino acid catabolism enzyme is a leucine catabolism enzyme. In another embodiment, the branched chain amino acid catabolism enzyme is an isoleucine catabolism enzyme. In another embodiment, the branched chain amino acid catabolism enzyme is a valine catabolism enzyme. In another embodiment, the branched chain amino acid catabolism enzyme is involved in the catabolism of leucine, isoleucine, and valine. In another embodiment, the branched chain amino acid catabolism enzyme is involved in the catabolism of leucine and valine, isoleucine and valine, or leucine and isoleucine. In one embodiment, the branched chain amino acid catabolism enzyme is an alpha-ketoisocaproic acid (MC) catabolism enzyme. In another embodiment, the branched chain amino acid catabolism enzyme is an α-ketoisovaleric acid (KIV) catabolism enzyme. In another embodiment, the branched chain amino acid catabolism enzyme is an α-keto-β-methylvaleric acid (KMV) catabolism enzyme. In another embodiment, the branched chain amino acid catabolism enzyme is involved in the catabolism of alpha-ketoisocaproic acid (MC), α-ketoisovaleric acid (KIV), and α-keto-β-methylvaleric acid (KMV). In another embodiment, the branched chain amino acid catabolism enzyme is involved in the catabolism of KIC and KIV, KIC and KMV, or MV and KMV.

In some embodiments, a branched chain amino acid catabolism enzyme is encoded by a gene encoding a branched chain amino acid catabolism enzyme derived from a bacterial species. In some embodiments, a branched chain amino acid catabolism enzyme is encoded by a gene encoding a branched chain amino acid catabolism enzyme derived from a non-bacterial species. In some embodiments, a branched chain amino acid catabolism enzyme is encoded by a gene derived from a eukaryotic species, e.g., a yeast species or a plant species. In one embodiment, the gene encoding the branched chain amino acid enzyme is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Pseudomonas, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartite, Nasonia vitipennis, Nostoc punctiforme, Pantoea ananatis, Pantoea agglomerans, Pectobacterium atrosepticum, Pectobacterium carotovorum, Pseudomonas aeruginosa, Psychrobacter anticus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae.

In one embodiment, the branched chain amino acid catabolism enzyme is an α-ketoisovalerate decarboxylase. As used herein “α-ketoisovalerate decarboxylase” or “alpha-ketoisovalerate decarboxylase” or “branched-chain α-keto acid decarboxylase” or “α-ketoacid decarboxylase” or “2-ketoisovalerate decarboxylase” (referred to herein also as KivD or ketoisovalerate decarboxylase) refers to any polypeptide having enzymatic activity that catalyzes the conversion of α-ketoisovalerate to isobutyraldehyde and carbon dioxide. α-ketoisovalerate decarboxylase sequences are available from many microorganism sources, including those disclosed herein. Alpha-ketoisovalerate decarboxylase employs the co-factor thiamine diphosphate (also known as thiamine pyrophosphate or “TPP” or “TDP”). Thiamine is the vitamin form of the co-factor which, when transported into a cell, is converted to thiamine diphosphate. Multiple distinct α-ketoisovalerate decarboxylase proteins are known in the art (see, e.g., US Pat. Appl. Publ. No. 2013/0203138, the entire contents of which are incorporated herein by reference).

In some embodiments, α-ketoisovalerate decarboxylase is encoded by an α-ketoisovalerate decarboxylase gene derived from a bacterial species. In some embodiments, α-ketoisovalerate decarboxylase is encoded by an α-ketoisovalerate decarboxylase gene derived from a non-bacterial species. In some embodiments, α-ketoisovalerate decarboxylase is encoded by an α-ketoisovalerate decarboxylase gene derived from a eukaryotic species, e.g., a yeast species or a plant species. In one embodiment, the α-ketoisovalerate decarboxylase gene is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Psychrobacter, Ralstonia, Saccharomyces, Salmonella, Sarcina, Serratia, Staphylococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartite, Nasonia vitipennis, Nostoc punctiforme, Pantoea ananatis, Pantoea agglomerans, Pectobacterium atrosepticum, Pectobacterium carotovorum, Psychrobacter anticus, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Yersinia enterocolitica, Yersinia mollaretii, Yersinia kristensenii, Yersinia rohdei, and Yersinia aldovae. In some embodiments, the α-ketoisovalerate decarboxylase is encoded by an α-ketoisovalerate decarboxylase gene derived from Lactococcus lactis. In another embodiment, the alpha-ketoisovalerate decarboxylase, e.g., kivD gene, is derived from Enterobacter cloacae (Accession No. P23234.1), Mycobacterium smegmatis (Accession No. A0R480.1), Mycobacterium tuberculosis (Accession NO. 053865.1), Mycobacterium avium (Accession No. Q742Q2.1), Azospirillum brasilense (Accession No. P51852.1), or Bacillus subtilis (see Oku et al., J. Biol. Chem. 263: 18386-96, 1988).

In one embodiment, the α-ketoisovalerate decarboxylase gene is a kivD gene. In another embodiment, the kivD gene is a Lactococcus lactis kivD gene.

Accordingly, in one embodiment, the kivD gene has at least about 80% identity with the sequence of SEQ ID NO:6. Accordingly, in one embodiment, the kivD gene has at least about 90% identity with the sequence of SEQ ID NO:6. Accordingly, in one embodiment, the kivD gene has at least about 95% identity with the sequence of SEQ ID NO:6. Accordingly, in one embodiment, the kivD gene has at least about 85%, 86%, 87%, 88%, 89%, 90%, 91%, 92%, 93%, 94%, 95%, 96%, 97%, 98%, or 99% identity with the sequence of SEQ ID NO:6. In another embodiment, the kivD gene comprises the sequence of SEQ ID NO:6. In yet another embodiment the kivD gene consists of the sequence of SEQ ID NO:6.

In another embodiment, the branched chain amino acid catabolism enzyme is a branched chain keto acid dehydrogenase (“BCKD”). As used herein “branched chain keto acid dehydrogenase” or “BCKD” refers to any polypeptide having enzymatic activity that oxidatively decarboxylates a branched chain keto acid into its respective acyl-CoA derivative. Multiple distinct branched chain keto acid dehydrogenases are known in the art and are available from many microorganism sources, including those disclosed herein, as well as eukaryotic sources. In bacteria, branched chain keto acid dehydrogenases are enzyme complexes that oxidatively decarboxylate all three branched chain keto acids into their respective acyl-CoA derivatives (see, for example, Massey et al., Bacteriol Rev., 40(1):42-54, 1976). Moreover, in mammals, dehydrogenases specific for 2-ketoisovalerate (EC 1.2.4.4) and 2-keto-3-methylvalerate and 2-keto-isocaproate (EC 1.2.4.3) have been identified (see, for example, Massey et al., Bacteriol Rev., 40(1):42-54, 1976). In one embodiment, the branched chain amino acid catabolism enzyme is a leucine catabolism enzyme.

In some embodiments, the branched chain amino acid catabolism enzyme is encoded by at least one gene encoding a branched chain amino acid catabolism enzyme derived from a bacterial species. In some embodiments, the branched chain amino acid catabolism enzyme is encoded by at least one gene encoding a branched chain amino acid catabolism enzyme derived from a non-bacterial species. In some embodiments, the branched chain amino acid catabolism enzyme is encoded by at least one gene derived from a eukaryotic species, e.g., a yeast species or a plant species. In another embodiment, the branched chain amino acid catabolism enzyme is encoded by at least one gene derived from a human.

In one embodiment, the at least one gene encoding the branched chain amino acid catabolism enzyme is derived from an organism of the genus or species that includes, but is not limited to, Acetinobacter, Azospirillum, Bacillus, Bacteroides, Bifidobacterium, Brevibacteria, Burkholderia, Citrobacter, Clostridium, Corynebacterium, Cronobacter, Enterobacter, Enterococcus, Erwinia, Helicobacter, Klebsiella, Lactobacillus, Lactococcus, Leishmania, Listeria, Macrococcus, Mycobacterium, Nakamurella, Nasonia, Nostoc, Pantoea, Pectobacterium, Proteus, Pseudomonas, Psychrobacter, Ralstonia, Saccharomyces, Salmonella, Sarcina, Serratia, Staphylococcus, Streptococcus, and Yersinia, e.g., Acetinobacter radioresistens, Acetinobacter baumannii, Acetinobacter calcoaceticus, Azospirillum brasilense, Bacillus anthracia, Bacillus cereus, Bacillus coagulans, Bacillus megaterium, Bacillus subtilis, Bacillus thuringiensis, Bacteroides fragilis, Bacteroides subtilis, Bacteroides thetaiotaomicron, Bifidobacterium bifidum, Bifidobacterium infantis, Bifidobacterium lactis, Bifidobacterium longum, Burkholderia xenovorans, Citrobacter youngae, Citrobacter koseri, Citrobacter rodentium, Clostridium acetobutylicum, Clostridium butyricum, Corynebacterium aurimucosum, Corynebacterium kroppenstedtii, Corynebacterium striatum, Cronobacter sakazakii, Cronobacter turicensis, Enterobacter cloacae, Enterobacter cancerogenus, Enterococcus faecium, Enterococcus faecalis, Erwinia amylovara, Erwinia pyrifoliae, Erwinia tasmaniensis, Helicobacter mustelae, Klebsiella pneumonia, Klebsiella variicola, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Leishmania infantum, Leishmania major, Leishmania brazilensis, Listeria grayi, Macrococcus caseolyticus, Mycobacterium avium, Mycobacterium intracellulare, Mycobacterium kansasii, Mycobacterium leprae, Mycobacterium marinum, Mycobacterium smegmatis, Mycobacterium tuberculosis, Mycobacterium ulcerans, Nakamurella multipartite, Nasonia vitipennis, Nostoc punctiforme, Pantoea ananatis, Pantoea agglomerans, Pectobacterium atrosepticum, Pectobacterium carotovorum, Pseudomonas putida, Pseudomonas aeruginosa, Psychrobacter anticus, Proteus vulgaris, Psychrobacter cryohalolentis, Ralstonia eutropha, Saccharomyces boulardii, Salmonella enterica, Sarcina ventriculi, Serratia odorifera, Serratia proteamaculans, Staphylococcus aerus, Staphylococcus capitis, Staphylococcys carnosus, Staphylococcus epidermidis, Staphylococcus hominis, Staphylococcus haemolyticus, Staphylococcus lugdunensis, Staphylococcus saprophyticus, Staphylococcus warneri, Streptococcus faecalis, Yersinia enterocolitica, Yersinia mollaretii, Yersinia kristensenii, Yersinia rohdei, and Yersinia aldovae. In some embodiments, the BCKD is encoded by at least one gene derived from Pseudomonas putida. In another embodiment, the BCKD is encoded by at least one gene derived from Pseudomonas aeruginosa. In another embodiment, the BCKD is encoded by at least one gene derived from Streptococcus faecalis. In another embodiment, the BCKD is encoded by at least one gene derived from Proteus vulgaris. In another embodiment, the BCKD is encoded by at least one gene derived from Bacillus subtilis. In another embodiment, the BCKD is encoded by at least one gene derived from Streptococcus faecalis. In another embodiment, the BCKD is encoded by at least one gene derived from Bacillus subtilis.

In one embodiment, the at least one gene encoding the branched chain keto acid dehydrogenase is a branched chain keto acid dehydrogenase gene from Pseudomonas aeruginosa PAO1. In one embodiment, the at least one gene encoding the branched chain keto acid dehydrogenase comprises the bkdA1-bkdA2-bkdB-lpdV operon. In one embodiment, the bkdA1-bkdA2-bkdB-lpdV operon is at least 90% identical to the uppercase sequence set forth in SEQ ID NO:7. In another embodiment, the bkdA1-bkdA2-bkdB-lpdV operon comprises the uppercase sequence set forth in SEQ ID NO:7. In another embodiment, the at least one gene encoding the branched chain keto acid dehydrogenase comprises the ldh-bkdA1-bkdA2-bkdB-lpdV operon. In one embodiment, the ldh-bkdA1-bkdA2-bkdB-lpdV operon is at least 90% identical to the uppercase sequence set forth in SEQ ID NO:8. In another embodiment, the ldh-bkdA1-bkdA2-bkdB-lpdV operon comprises the uppercase sequence as set forth in SEQ ID NO:8. In another embodiment, the at least one gene encoding the branched chain keto acid dehydrogenase is 2-ketoisovalerate (EC 1.2.4.4). In another embodiment, the at least one gene encoding the branched chain keto acid dehydrogenase is 2-keto-3-methylvalerate and 2-keto-isocaproate (EC 1.2.4.3). In yet another embodiment, the at least one gene encoding the branched chain keto acid dehydrogenase is the human dehydrogenase/decarboxylase (E1). In another embodiment, the at least one gene encoding the branched chain keto acid dehydrogenase comprises the human E1α and two E1β subunits. In another embodiment, the at least one gene encoding the branched chain keto acid dehydrogenase comprises the human dihydrolipoyl transacylase (E2) gene. In yet another embodiment, the at least one gene encoding the branched chain keto acid dehydrogenase comprises the human dihydrolipoamide dehydrogenase (E3) gene. In another embodiment, the at least one gene encoding the branched chain keto acid dehydrogenase comprises the human dehydrogenase/decarboxylase (E1) gene, the human dihydrolipoly transacylase (E2) gene, and the human dihydrolipoamide dehydrogenase (E3) gene.

In one embodiment, the at least one gene encoding the branched chain keto acid dehydrogenase comprises the bkdA1-bkdA2-bkdB-lpdV operon. In one embodiment, the at least one BCKD gene has at least about 80% identity with the entire uppercase sequence of SEQ ID NO:7. Accordingly, in one embodiment, the at least one BCKD gene has at least about 90% identity with the entire uppercase sequence of SEQ ID NO:7. Accordingly, in one embodiment, the at least one BCKD gene has at least about 95% identity with the entire uppercase sequence of SEQ ID NO:7. Accordingly, in one embodiment, the at least one BCKD gene has at least about 85%, 86%, 87%, 88%, 89%, 90%, 91%, 92%, 93%, 94%, 95%, 96%, 97%, 98%, or 99% identity with the entire uppercase sequence of SEQ ID NO:7. In another embodiment, the at least one BCKD gene comprises the uppercase sequence of SEQ ID NO:7. In yet another embodiment the at least one BCKD gene consists of the uppercase sequence of SEQ ID NO:7.

In another embodiment, the at least one gene encoding the branched chain keto acid dehydrogenase comprises the ldh-bkdA1-bkdA2-bkdB-lpdV operon. In another embodiment, the at least one BCKD gene is coexpressed with an additional branched chain amino acid dehydrogenase. In one embodiment, the at least one BCKD gene is coexpressed with a leucine dehydrogenase, e.g., ldh. In one embodiment, the ldh gene has at least about 80% identity with the entire uppercase sequence of SEQ ID NO:8. Accordingly, in one embodiment, the ldh gene has at least about 90% identity with the entire uppercase sequence of SEQ ID NO:8. Accordingly, in one embodiment, the ldh gene has at least about 95% identity with the entire uppercase sequence of SEQ ID NO:8. Accordingly, in one embodiment, the ldh gene has at least about 85%, 86%, 87%, 88%, 89%, 90%, 91%, 92%, 93%, 94%, 95%, 96%, 97%, 98%, or 99% identity with the entire uppercase sequence of SEQ ID NO:8. In another embodiment, the ldh gene comprises the uppercase sequence of SEQ ID NO:8. In yet another embodiment the ldh gene consists of the uppercase sequence of SEQ ID NO:8.

In some embodiments, the branched chain amino acid catabolism enzyme is used to convert a branched chain amino acid, e.g., leucine, valine, isoleucine, to its corresponding α-keto-acid, e.g., α-ketoisocaproate, α-keto-β-methylvalerate, and α-ketoisovalerate. In some embodiments, wherein a branched chain amino acid catabolism enzyme is used to convert a branched chain amino acid, e.g., leucine, valine, isoleucine, to its corresponding α-keto-acid, the engineered bacteria further comprise one or more branched chain amino acid catabolism enzyme(s) to convert an α-keto-acid to its corresponding acetyl CoA, e.g., isovaleryl-CoA, α-methylbutyryl-CoA, and isobutyryl-CoA. In some embodiments, wherein a branched chain amino acid catabolism enzyme is used to convert a branched chain amino acid, e.g., leucine, valine, isoleucine, to its corresponding α-keto-acid, the engineered bacteria further comprise one or more branched chain amino acid catabolism enzyme(s) to convert an α-keto-acid to its corresponding aldehyde, e.g., isovaleraldehyde, isobutyraldehyde, and 2-methylbutyraldehyde. In some embodiments, the engineered bacteria may further comprise an alcohol dehydrogenase enzyme in order to convert the branched chain amino acid-derived aldehyde (e.g., isovaleraldehyde, isobutyraldehyde, 2-methylbutyraldehyde) to its respective alcohol. In some embodiments, the engineered bacteria may further comprise an aldehyde dehydrogenase enzyme in order to convert the branched chain amino acid-derived aldehyde (e.g., isovaleraldehyde, isobutyraldehyde, 2-methylbutyraldehyde) to its respective carboxylic acid.

Enzymes involved in the catabolism of branched chain amino acids are well known to those of skill in the art. For example, in bacteria, leucine dehydrogenase (LeuDH), branched achain amino acid transferase (IlvE), amino acid oxidase (also known as amino acid deaminase) (L-AAD), as well as other known enzymes, can be used to convert a BCAA to its corresponding α-keto acid, e.g., ketoisocaproate (KIC), ketoisovalerate (KIV), and ketomethylvalerate (KMV). Leucine dehydrogenases, branched chain amino acid transamination enzymes (EC 2.6.1.42), and L-amino acid deaminases (L-AAD), which oxidatively deaminate branched chain amino acids into their respective alpha-keto acid, are known (Baker et al., Structure, 3(7):693-705, 1995; Peng et al., J. Bact., 139(2):339-45, 1979; and Kline et al., J. Bact., 130(2):951-3, 1977). In bacteria, branched chain keto acid dehydrogenases (“BCKDs”) are enzyme complexes that oxidatively decarboxylate all three branched chain keto acids into their respective acyl-CoA derivatives. Thus, in one embodiment, the branched chain amino acid catabolism enzyme is a branched chain keto acid dehydrogenase (BCKD). Moreover, in mammals, dehydrogenases specific for 2-ketoisovalerate (EC 1.2.4.4) and 2-keto-3-methylvalerate and 2-keto-isocaproate (EC 1.2.4.3) have been identified (see, for example, Massey et al., Bacteriol Rev., 40(1):42-54, 1976). In bacteria, branched chain keto acid dehydrogenases (“BCKDs”) are enzyme complexes that oxidatively decarboxylate all three branched chain keto acids into their respective acyl-CoA derivatives. Also, for example, in bacteria, α-ketoisovalerate decarboxylase (KivD) enzymes are capable of converting α-keto acids into aldehydes (e.g., isovaleraldehyde, isobutyraldehyde, 2-methylbutyraldehyde). Specifically, the α-ketoisovalerate decarboxylase enzyme KivD is capable of metabolizing valine by converting α-ketoisovalerate to isobutyraldehyde (see, for example, de la Plaza et al., FEMS Microbiol. Lett. 2004, 238(2):367-374). KivD is capable of metabolizing leucine by converting α-ketoisocaproate (MC) to isovaleraldehyde. KivD is also capable of metabolizing isoleucine by converting α-ketomethylvalerate (KMV) to 2-methylbutyraldehyde. In addition, enzymes for converting isovaleraldehyde, isobutyraldehyde, and 2-methylbutyraldehyde to their respective alcohols or carboxylic acids are known and available. For example, alcohol dehydrogenases (e.g., Adh2, YqhD) can convert isovaleraldehyde, isobutyraldehyde, 2-methylbutyraldehyde to isopentanol, isobutanol, and 2-methylbutanol, respectively. Aldehyde dehydrogenases (e.g., PadA) can convert isovaleraldehyde, isobutyraldehyde, 2-methylbutyraldehyde to isovalerate, isobutyrate, and 2-methylbutyrate, respectively.

In some embodiments, the branched chain amino acid catabolism enzyme increases the rate of branched chain amino acid catabolism. In some embodiments, the branched chain amino acid catabolism enzyme decreases the level of one or more branched chain amino acids, e.g., leucine, isoleucine, and/or valine, in a cell, tissue, or organism. In some embodiments, the branched chain amino acid catabolism enzyme decreases the level of alpha-keto acid derived from BCAA in a cell, tissue, or organism. In some embodiments, the branched chain amino acid catabolism enzyme decreases the level of branched chain amino acid as compared to the level of its corresponding alpha-keto acid in a cell, tissue, or organism. In other embodiments, the branched chain amino acid catabolism enzyme increases the level of alpha-keto acid as compared to the level of its corresponding branched chain amino acid in a cell, tissue, or organism. In some embodiments, the branched chain amino acid catabolism enzyme decreases the level of the branched chain amino acid as compared to the level of its corresponding Acyl-CoA derivative in a cell, tissue, or organism. In some embodiments, the branched chain amino acid catabolism enzyme increases the level of the Acyl-CoA derivative as compared to the level of the branched chain amino acid in a cell, tissue, or organism. In some embodiments, the branched chain amino acid catabolism enzyme decreases the level of alpha-keto aldehyde derived from BCAA, e.g., isovaleraldehyde, isobutyraldehyde, and 2-methylbutyraldehyde, in a cell, tissue, or organism. In some embodiments, the branched chain amino acid catabolism enzyme decreases the level of branched chain amino acid as compared to the level of its corresponding alpha-keto aldehyde, e.g., isovaleraldehyde, isobutyraldehyde, and 2-methylbutyraldehyde, in a cell, tissue, or organism. In other embodiments, the branched chain amino acid catabolism enzyme increases the level of alpha-keto aldehyde, e.g., isovaleraldehyde, isobutyraldehyde, and 2-methylbutyraldehyde, as compared to the level of its corresponding branched chain amino acid in a cell, tissue, or organism. In some embodiments, the branched chain amino acid catabolism enzyme decreases the level of a corresponding downstream metabolite, e.g., isovalerate, isobutyrate, 2-methylbutyrate, isopentanol, isobutanol, and 2-methylbutanol, in a cell, tissue, or organism. In some embodiments, the branched chain amino acid catabolism enzyme decreases the level of branched chain amino acid as compared to the level of a corresponding downstream metabolite, e.g., isovalerate, isobutyrate, 2-methylbutyrate, isopentanol, isobutanol, and 2-methylbutanol, in a cell, tissue, or organism. In other embodiments, the branched chain amino acid catabolism enzyme increases the level of a downstream metabolite, e.g., isovalerate, isobutyrate, 2-methylbutyrate, isopentanol, isobutanol, and 2-methylbutanol, as compared to the level of its corresponding branched chain amino acid in a cell, tissue, or organism.

In some embodiments, the branched chain amino acid catabolism enzyme is a leucine catabolism enzyme. In other embodiments, the branched chain amino acid catabolism enzyme is an isoleucine catabolism enzyme. In other embodiments, the branched chain amino acid catabolism enzyme is a valine catabolism enzyme. In some embodiments, the branched chain amino acid catabolism enzyme is involved in the catabolism of leucine, isoleucine, and valine. In another embodiment, the branched chain amino acid catabolism enzyme is involved in the catabolism of leucine and valine, isoleucine and valine, or leucine and isoleucine. In some embodiments, the branched chain amino acid catabolism enzyme converts leucine, isoleucine, and/or valine into its corresponding α-keto acid. In certain specific embodiments, the present disclosure provides an engineered bacteria comprising gene sequence(s) encoding one or more catabolism enzymes selected from leucine dehydrogenase (LeuDH), BCAA aminotransferase (IlvE), and/or amino acid oxidase (L-AAD).

In some embodiments, the branched chain amino acid catabolism enzyme is an alpha-ketoisocaproic acid (KIC) catabolism enzyme. In other embodiments, the branched chain amino acid catabolism enzyme is an alpha-ketoisovaleric acid (KIV) catabolism enzyme. In other embodiments, the branched chain amino acid catabolism enzyme is an alpha-keto-beta-methylvaleric acid (KMV) catabolism enzyme. In other embodiments, the branched chain amino acid catabolism enzyme is involved in the catabolism of alpha-ketoisocaproic acid (MC), alpha-ketoisovaleric acid (KIV), and alpha-keto-beta-methylvaleric acid (KMV). In other embodiments, the branched chain amino acid catabolism enzyme is involved in the catabolism of KIC and KIV, KIC and KMV, or KIV and KMV. In some embodiments, the branched chain amino acid catabolism enzyme converts alpha-ketoisocaproic acid (MC), alpha-ketoisovaleric acid (KIV), and/or alpha-keto-beta-methylvaleric acid (KMV) into its corresponding aldehyde, e.g., isovaleraldehyde, isobutyraldehyde, and/or 2-methylbutyraldehyde. In some embodiments, the present disclosure provides an engineered bacteria comprising gene sequence(s) encoding KivD.

In one embodiment, the branched chain amino acid catabolism enzyme is an isovaleraldehyde catabolism enzyme. In another embodiment, the branched chain amino acid catabolism enzyme is an isobutyraldehyde catabolism enzyme. In another embodiment, the branched chain amino acid catabolism enzyme is 2-methylbutyraldehyde catabolism enzyme. In another embodiment, the branched chain amino acid catabolism enzyme is involved in the catabolism of isovaleraldehyde, isobutyraldehyde, and 2-methylbutyraldehyde. In another embodiment, the branched chain amino acid catabolism enzyme is involved in the catabolism of isovaleraldehyde and isobutyraldehyde, isovaleraldehyde and 2-methylbutyraldehyde, or isobutyraldehyde and 2-methylbutyraldehyde. In some embodiments, the present disclosure provides an engineered bacteria comprising gene sequence(s) encoding one or more alcohol dehydrogenase(s), e.g., Ahd2, YqhD. In some embodiments, the present disclosure provides an engineered bacteria comprising gene sequence(s) encoding one or more aldehyde dehydrogenase(s), e.g., PadA. In some embodiments, the present disclosure provides an engineered bacteria comprising gene sequence(s) encoding one or more alcohol dehydrogenase(s), e.g., Ahd2, YqhD and one or more aldehyde dehydrogenase(s), e.g., PadA.

In some embodiments, the present disclosure provides an engineered bacteria comprising gene sequence(s) encoding one or more branched chain amino acid catabolism enzyme(s) involved in the catabolism of leucine, isoleucine, and/or valine, and further comprises gene sequence(s) encoding one or more branched chain amino acid catabolism enzyme(s) involved in the catabolism of KIC, MV, and/or KMV. In some embodiments, the present disclosure provides an engineered bacteria comprising gene sequence(s) encoding one or more branched chain amino acid catabolism enzyme(s) involved in the catabolism of leucine, isoleucine, and/or valine, further comprises gene sequence(s) encoding one or more branched chain amino acid catabolism enzyme(s) involved in the catabolism of MC, MV, and/or KMV, and further comprises gene sequence(s) encoding one or more branched chain amino acid catabolism enzyme(s) involved in the catabolism of. isovaleraldehyde, isobutyraldehyde, and/or 2-methylbutyraldehyde. In some embodiments, the present disclosure provides an engineered bacteria comprising gene sequence(s) encoding one or more branched chain amino acid catabolism enzyme(s) selected from LeuDH, IlvE, L-AAD, KivD, PadA, Adh2, and YqhD.

In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively. In some embodiments, the present disclosure provides an engineered bacteria comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively. In some embodiments, the present disclosure provides an engineered bacteria comprising gene sequence(s) encoding one or more branched chain amino acid catabolism enzyme(s) involved in the conversion of isovaleraldehyde, isobutyraldehyde, and/or 2-methylbutyraldehyde to isovalerate, isobutyrate, and/or 2-methylbutyrate, respectively. In some embodiments, the present disclosure provides an engineered bacteria comprising gene sequence(s) encoding one or more branched chain amino acid catabolism enzyme(s) involved in the conversion of, isovaleraldehyde, isobutyraldehyde, and/or 2-methylbutyraldehyde to isopentanol, isobutanol, and/or 2-methylbutanol respectively.

In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, and further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively. In some embodiments, the present disclosure provides an engineered bacteria comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of, isovaleraldehyde, isobutyraldehyde, and/or 2-methylbutyraldehyde to isovalerate, isobutyrate, and/or 2-methylbutyrate, respectively. In some embodiments, the present disclosure provides an engineered bacteria comprising gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of isovaleraldehyde, isobutyraldehyde, and/or 2-methylbutyraldehyde to isopentanol, isobutanol, and/or 2-methylbutanol respectively.

In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of isovaleraldehyde, isobutyraldehyde, and/or 2-methylbutyraldehyde to isovalerate, isobutyrate, and/or 2-methylbutyrate, respectively. In some embodiments, the present disclosure provides an engineered bacteria comprising gene sequence(s) encoding one or more branched chain amino acid catabolism enzyme(s) involved in the conversion of leucine, isoleucine, and/or valine to KIC, KIV, and/or KMV, respectively, further comprises gene sequence(s) encoding one or more branched chain amino acid catabolism enzyme(s) involved in the conversion of KIC, KIV, and/or KMV to isovaleraldehyde, isobutyraldehyde, and/or 2-methylbutyraldehyde, respectively, and further comprises gene sequence(s) encoding one or more branched chain amino acid catabolism enzyme(s) involved in the conversion of, isovaleraldehyde, isobutyraldehyde, and/or 2-methylbutyraldehyde to isopentanol, isobutanol, and/or 2-methylbutanol, respectively. In some embodiments, the present disclosure provides an engineered bacteria comprising gene sequence(s) encoding one or more branched chain amino acid catabolism enzyme(s) selected from LeuDH, IlvE, and/or L-AAD, KivD, PadA, Adh2, and YqhD.

Enzymes involved in the catabolism of a branched chain amino acid may be expressed or modified in the bacteria disclosed herein in order to enhance catabolism of a branched chain amino acid, e.g., leucine. Specifically, when a branched chain amino acid catabolism enzyme is expressed in the engineered bacteria disclosed herein, the engineered bacteria are able to convert (deaminate) more branched chain amino acids (e.g., leucine, valine, isoleucine) into their respective alpha-keto acids (KIC, KIV, KMV) and/or convert more BCAA alpha-keto acids (e.g., KIC, KIV, KMV) into respective BCAA-derived aldehydes (e.g., isovaleraldehyde, isobutyraldehyde, 2-methylbutyraldehyde) and/or convert more BCAA-derived aldehydes into respective alcohols (e.g., isopentanol, isobutanol, 2-methylbutanol) and/or convert more BCAA-derived aldehydes into respective carboxylic acids (isovalerate, isobutyrate, 2-methylbutyrate), and/or convert (decarboxylate) more branched chain alpha-keto acids into their respective acyl-CoA derivatives when the catabolism enzyme(s) is expressed, in comparison with unmodified bacteria of the same bacterial subtype under the same conditions. Thus, for example, the genetically engineered bacteria comprising gene sequence encoding a branched chain amino acid catabolism enzyme can catabolize the branched chain amino acid, e.g., leucine, and/or its corresponding alpha-keto acid, e.g., alpha-ketoisocaproate, to treat diseases associated with catabolism of branched chain amino acids, such as obesity related insulin resistance, T2D and other disorders described herein.

In some embodiments, the engineered bacteria comprise gene sequence(s) encoding one or more branched chain amino acid catabolism enzyme(s) and gene sequence(s) encoding one or more transporter(s) capable of importing a BCAA or metabolite thereof. In some embodiments, the engineered bacteria comprise gene sequence(s) encoding at least one branched chain amino acid catabolism enzyme and gene sequence(s) encoding two or more copies of a transporter capable of importing a BCAA or metabolite thereof. In some embodiments, the engineered bacteria comprise gene sequence(s) encoding at least one branched chain amino acid catabolism enzyme and gene sequence(s) encoding two or more different transporter(s) capable of importing a BCAA or metabolite thereof. In certain embodiments, the transporter is a leucine transporter. In certain embodiments, the transporter is a valine transporter. In certain embodiments, the transporter is an isoleucine transporter. In certain embodiments, the transporter is a branched chain amino acid transporter, e.g., capable of importing leucine, isoleucine, and valine. In certain specific embodiments, the transporter is selected from LivKHMGF and BrnQ.

In some embodiments, the engineered bacteria comprise gene sequence(s) encoding at least one branched chain amino acid catabolism enzyme and gene sequence(s) encoding one or more BCAA binding proteins, e.g., a BCAA binding protein that assists in bringing BCAA(s) into the bacterial cell. In some embodiments, the engineered bacteria comprise gene sequence(s) encoding at least one branched chain amino acid catabolism enzyme, gene sequence(s) encoding one or more transporter(s) capable of importing one or more BCAAs, and gene sequence(s) encoding one or more BCAA binding proteins, e.g., a BCAA binding protein that assists in bringing BCAA(s) into the bacterial cell. In any of these embodiments, the engineered bacteria comprise gene sequence(s) encoding two or more copies of a BCAA binding protein. In any of these embodiments, the engineered bacteria comprise gene sequence(s) encoding two or more different BCAA binding proteins. In certain embodiments, the BCAA binding protein is LivJ.

In any of the embodiments described above and herein, the engineered bacteria may further comprise one or more genetic modification(s) that reduces export of a branched chain amino acid from the bacteria, e.g., a deletion or mutation in at least one gene associated with the export of a BCAA, e.g., deletion or mutation in leuE gene and/or its promoter (which reduces or eliminates the export of leucine). In some embodiments, the engineered bacteria comprise gene sequence(s) encoding at least one branched chain amino acid catabolism enzyme and at least one genetic modification that reduces export of a branched chain amino acid. In certain specific embodiments, the engineered bacteria comprise gene sequence(s) encoding at least one branched chain amino acid catabolism enzyme, gene sequence(s) encoding one or more transporter(s) capable of importing a BCAA, and at least one genetic modification that reduces export of a branched chain amino acid. In certain specific embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, and at least one genetic modification that reduces export of a branched chain amino acid. In some embodiments, the engineered bacteria comprise gene sequence(s) encoding at least one branched chain amino acid catabolism enzyme, gene sequence(s) encoding one or more BCAA binding proteins, and at least one genetic modification that reduces export of a branched chain amino acid. In any of these embodiments, the genetic modification may be a deletion or mutation in one or more gene(s) that allow or assist in the export of a BCAA. In any of these embodiment, the genetic modification may be a deletion or mutation in a leuE gene and/or its promoter.

In any of the embodiments described above and herein, the engineered bacteria comprise gene sequence(s) encoding one or more branched chain amino acid catabolism enzyme(s), and at least one genetic modification that reduces endogenous biosynthesis of a branched chain amino acid, for example, a deletion or mutation in at least one gene required for BCAA synthesis, e.g., deletion or mutation in ilvC gene and/or its promoter, which gene is required for BCAA synthesis and whose absence creates an auxotroph requiring the bacterial cell to import leucine. In some embodiments, the engineered bacteria comprise gene sequence(s) encoding one or more branched chain amino acid catabolism enzyme(s), gene sequence(s) encoding one or more transporter(s) capable of importing a BCAA, and at least one genetic modification that reduces endogenous biosynthesis of a branched chain amino acid, for example, a deletion or mutation in at least one gene required for BCAA synthesis. In some embodiments, the engineered bacteria comprise gene sequence(s) encoding at least one branched chain amino acid catabolism enzyme, gene sequence(s) encoding one or more BCAA binding proteins, and at least one genetic modification that reduces endogenous biosynthesis of a branched chain amino acid. In some embodiments, the engineered bacteria comprise gene sequence(s) encoding at least one branched chain amino acid catabolism enzyme, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more BCAA binding proteins, at least one genetic modification that reduces export of a branched chain amino acid, and at least one genetic modification that reduces endogenous biosynthesis of a branched chain amino acid. In some embodiments, the engineered bacteria comprise gene sequence(s) encoding at least one branched chain amino acid catabolism enzyme, gene sequence(s) encoding one or more transporter(s) capable of importing a BCAA, gene sequence(s) encoding one or more BCAA binding proteins, at least one genetic modification that reduces export of a branched chain amino acid, and at least one genetic modification that reduces endogenous biosynthesis of a branched chain amino acid. In any of these embodiments, the at least one genetic modification that reduces endogenous biosynthesis of a branched chain amino acid can be a deletion or mutation in at least one gene required for BCAA synthesis, e.g., deletion or mutation in ilvC gene and/or its promoter.

In any of the embodiments described above and herein, the gene sequence(s) encoding at least one branched chain amino acid catabolism enzyme, and/or gene sequence(s) encoding one or more transporter(s) capable of importing a BCAA, and/or gene sequence(s) encoding one or more BCAA binding proteins, and/or other sequence can be present in the bacterial chromosome. In any of the embodiments described above and herein, the gene sequence(s) encoding at least one branched chain amino acid catabolism enzyme, and/or gene sequence(s) encoding one or more transporter(s) capable of importing a BCAA, and/or gene sequence(s) encoding one or more BCAA binding proteins, and/or other sequence can be present in one or more plasmids.

The present disclosure further comprises genes encoding functional fragments of a branched chain amino acid catabolism enzyme or functional variants of the branched chain amino acid catabolism enzyme.

Branched chain amino acid catabolism can be assessed using the coupled enzymatic assay method as described by Zhang et al. (see, for example, Zhang et al., Proc. Natl. Acad. Sci., 105(52):20653-58, 2008). Furthermore, catabolism of branched chain amino acids can also be assessed in vitro by measuring the disappearance of α-ketoisovalerate as described by de la Plaza (see, for example, de la Plaza et al., FEMS Microbiol. Letters, 2004, 238(2):367-374).

In one embodiment, the bacterial cell comprises a heterologous gene encoding at least one branched chain amino acid catabolism enzyme. In one embodiment, the bacterial cell comprises a heterologous gene encoding a transporter of a branched chain amino acid and a heterologous gene encoding a branched chain amino acid catabolism enzyme. In one embodiment, the bacterial cell comprises a heterologous gene encoding a branched chain amino acid catabolism enzyme and a genetic modification that reduces export of branched chain amino acids. In one embodiment, the bacterial cell comprises a heterologous gene encoding a transporter of branched chain amino acids, a heterologous gene encoding a branched chain amino acid catabolism enzyme, and a genetic modification that reduces export of branched chain amino acids. Transporters and exporters are described in more detail in the subsections, below.

B. Transporters of Branched Chain Amino Acids

Branched chain amino acid transporters may be expressed or modified in the recombinant bacteria described herein in order to enhance branched chain amino acid transport into the cell. Specifically, when the transporter of branched chain amino acids is expressed in the recombinant bacterial cells described herein, the bacterial cells import more branched chain amino acids into the cell when the transporter is expressed than unmodified bacteria of the same bacterial subtype under the same conditions. Thus, the genetically engineered bacteria comprising a heterologous gene encoding transporter of branched chain amino acids may be used to import one or more branched chain amino acids into the bacteria so that any gene encoding a branched chain amino acid catabolism enzyme expressed in the organism can catabolize the branched chain amino acid to treat a disease associated with amino acid metabolism, such as cancer.

The uptake of amino acids into bacterial cells is mediated by proteins well known to those of skill in the art. For example, two well characterized BCAA transport systems have been characterized in several bacteria, including Escherichia coli. BCAAs are transported by two systems into bacterial cells (i.e., imported), the osmotic-shock-sensitive systems designated LIV-I and LS (leucine-specific), and by an osmotic-shock resistant system, BrnQ, formerly known as LIV-II (see Adams et al., J. Biol. Chem. 265:11436-43 (1990); Anderson and Oxender, J. Bacteriol. 130:384-92 (1977); Anderson and Oxender, J. Bacteriol. 136:168-74 (1978); Haney et al., J. Bacteriol. 174:108-15 (1992); Landick and Oxender, J. Biol. Chem. 260:8257-61 (1985); Nazos et al., J. Bacteriol. 166:565-73 (1986); Nazos et al., J. Bacteriol. 163:1196-202 (1985); Oxender et al., Proc. Natl. Acad. Sci. USA 77:1412-16 (1980); Quay et al., J. Bacteriol. 129:1257-65 (1977); Rahmanian et al., J. Bacteriol. 116:1258-66 (1973); Wood, J. Biol. Chem. 250:4477-85 (1975); Guardiola et al., J. Bacteriol. 117:393-405 (1974); Guardiola and Iaccarino, J. Bacteriol. 108:1034-44 (1971); Ohnishi et al., Jpn. J. Genet. 63:343-57)(1988); Yamato and Anraku, J. Bacteriol. 144:36-44 (1980); and Yamato et al., J. Bacteriol. 138:24-32 (1979)). Transport by the BrnQ system is mediated by a single membrane protein. Transport mediated by the LIV-I system is dependent on the substrate binding protein LivJ (also known as LIV-BP), while transport mediated by LS system is mediated by the substrate binding protein LivK (also known as LS-BP). LivJ is encoded by the livJ gene, and binds isoleucine, leucine and valine with K_{d} values of ˜10⁻⁶ and ˜10⁻⁷ M, while LivK is encoded by the livK gene, and binds leucine with a K_{d} value of ˜10⁻⁶ M (See Landick and Oxender, J. Biol. Chem. 260:8257-61 (1985)). Both LivJ and LivK interact with the inner membrane components LivHMGF to enable ATP-hydrolysis-coupled transport of their substrates into the cell, forming the LIV-I and LS transport systems, respectively. The LIV-I system transports leucine, isoleucine and valine, and to a lesser extent serine threonine and alanine, whereas the LS system only transports leucine. The six genes encoding the E. coli LIV-I and LS systems are organized into two transcriptional units, with livKHMGF transcribed as a single operon, and livJ transcribed separately. The Escherichia coli liv genes can be grouped according to protein function, with the livJ and livK genes encoding periplasmic binding proteins with the binding affinities described above, the livH and livM genes encoding inner membrane permeases, and the livG and livF genes encoding cytoplasmic ATPases.

In one embodiment, the at least one gene encoding a transporter of a branched chain amino acid is the brnQ gene. In one embodiment, the at least one gene encoding a transporter of a branched chain amino acid is the livJ gene. In one embodiment, the at least one gene encoding a transporter of branched chain amino acid is the livH gene. In one embodiment, the at least one gene encoding a transporter of branched chain amino acid is the livM gene. In one embodiment, the at least one gene encoding a transporter of branched chain amino acid is the livG gene. In one embodiment, the at least one gene encoding a transporter of branched chain amino acid is the livF gene. In one embodiment, the at least one gene encoding a transporter of an amino acid is the livKHMGF operon. In one embodiment, the at least one gene encoding a transporter of an amino acid is the livK gene. In another embodiment, the livKHMGF operon is an Escherichia coli livKHMGF operon. In another embodiment, the at least one gene encoding a transporter of an amino acid comprises the livKHMGF operon and the livJ gene. In one embodiment, the bacterial cell of the invention has been genetically engineered to comprise at least one heterologous gene encoding a LIV-I system. In one embodiment, the bacterial cell of the invention has been genetically engineered to comprise at least one heterologous gene encoding a LS system. In one embodiment, the bacterial cell of the invention has been genetically engineered to comprise at least one heterologous gene encoding a LIV-I system. In one embodiment, the bacterial cell of the invention has been genetically engineered to comprise at least one heterologous livJ gene, and at least one heterologous gene selected from the group consisting of livH, livM, livG, and livF. In one embodiment, the bacterial cell of the invention has been genetically engineered to comprise at least one heterologous livK gene, and at least one heterologous gene selected from the group consisting of livH, livM, livG, and livF.

In one embodiment, the branched chain amino acid transporter gene has at least about 80% identity with the uppercase sequence of SEQ ID NO:9. Accordingly, in one embodiment, the branched chain amino acid transporter gene has at least about 90% identity with the uppercase sequence of SEQ ID NO:9. Accordingly, in one embodiment, the branched chain amino acid transporter gene has at least about 95% identity with the uppercase sequence of SEQ ID NO:9. Accordingly, in one embodiment, the branched chain amino acid transporter gene has at least about 85%, 86%, 87%, 88%, 89%, 90%, 91%, 92%, 93%, 94%, 95%, 96%, 97%, 98%, or 99% identity with the uppercase sequence of SEQ ID NO:9. In another embodiment, the branched chain amino acid transporter gene comprises the uppercase sequence of SEQ ID NO:9. In yet another embodiment the branched chain amino acid transporter gene consists of the uppercase sequence of SEQ ID NO:9.

In one embodiment, the branched chain amino acid transporter gene has at least about 80% identity with the sequence of SEQ ID NO:10. Accordingly, in one embodiment, the branched chain amino acid transporter gene has at least about 90% identity with the sequence of SEQ ID NO:10. Accordingly, in one embodiment, the branched chain amino acid transporter gene has at least about 95% identity with the sequence of SEQ ID NO:10. Accordingly, in one embodiment, the branched chain amino acid transporter gene has at least about 85%, 86%, 87%, 88%, 89%, 90%, 91%, 92%, 93%, 94%, 95%, 96%, 97%, 98%, or 99% identity with the sequence of SEQ ID NO:10. In another embodiment, the branched chain amino acid transporter gene comprises the sequence of SEQ ID NO:10. In yet another embodiment the branched chain amino acid transporter gene consists of the sequence of SEQ ID NO:10.

In some embodiments, the transporter of one or more branched chain amino acids is encoded by a transporter of the one or more branched chain amino acids gene derived from a bacterial genus or species, including but not limited to, Escherichia coli. In some embodiments, the bacterial species is Escherichia coli. In some embodiments, the bacterial species is Escherichia coli strain Nissle.

Assays for testing the activity of a transporter of a branched chain amino acid, a functional variant of a transporter of a branched chain amino acid, or a functional fragment of transporter of a branched chain amino acid are well known to one of ordinary skill in the art. For example, import of an amino acid may be determined using the methods as described in Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of each of which are expressly incorporated by reference herein.

In one embodiment, when the transporter of a branched chain amino acid is expressed in the recombinant bacterial cells described herein, the bacterial cells import 10% more branched chain amino acid into the bacterial cell when the transporter is expressed than unmodified bacteria of the same bacterial subtype under the same conditions. In another embodiment, when the transporter of branched chain amino acids is expressed in the recombinant bacterial cells described herein, the bacterial cells import 20%, 30%, 40%, 50%, 60%, 70%, 80%, 90% or 100% more branched chain amino acids into the bacterial cell when the transporter is expressed than unmodified bacteria of the same bacterial subtype under the same conditions. In yet another embodiment, when the transporter of branched chain amino acids is expressed in the recombinant bacterial cells described herein, the bacterial cells import two-fold more branched chain amino acids into the cell when the transporter is expressed than unmodified bacteria of the same bacterial subtype under the same conditions. In yet another embodiment, when the transporter of branched chain amino acids is expressed in the recombinant bacterial cells described herein, the bacterial cells import three-fold, four-fold, five-fold, six-fold, seven-fold, eight-fold, nine-fold, or ten-fold more branched chain amino acids into the cell when the transporter is expressed than unmodified bacteria of the same bacterial subtype under the same conditions.

C. Exporters of Branched Chain Amino Acids

Branched chain amino acid exporters may be modified in the recombinant bacteria described herein in order to reduce branched chain amino acid export from the cell. Specifically, when the recombinant bacterial cells described herein comprise a genetic modification that reduces export of branched chain amino acids, the bacterial cells retain more branched chain amino acids in the bacterial cell than unmodified bacteria of the same bacterial subtype under the same conditions. Thus, the recombinant bacteria comprising a genetic modification that reduces export of branched chain amino acids may be used to retain more branched chain amino acids in the bacterial cell so that any branched chain amino acid catabolism enzyme expressed in the organism can catabolize the branched chain amino acid(s).

The export of amino acids from bacterial cells is mediated by proteins well known to those of skill in the art. For example, one branched chain amino acid exporter, the leucine exporter LeuE has been characterized in Escherichia coli (Kutukova et al., FEBS Letters 579:4629-34 (2005); incorporated herein by reference). LeuE is encoded by the leuE gene in Escherichia coli (also known as yeaS) (SEQ ID NO:11). Additionally, a two-gene encoded exporter of the branched chain amino acids isoleucine, valine and leucine, denominated BrnFE was identified in the bacteria Corynebacterium glutamicum (Kennerknecht et al., J. Bacteriol. 184:3947-56 (2002); incorporated herein by reference). The BrnFE system is encoded by the Corynebacterium glutamicum genes brnF and brnE, and homologues of said genes have been identified in several organisms, including Agrobacterium tumefaciens, Achaeoglobus fulgidus, Bacillus subtilis, Deinococcus radiodurans, Escherichia coli, Haemophilus influenzae, Helicobacter pylori, Lactococcus lactis, Streptococcus pneumoniae, and Vibrio cholerae (see Kennerknecht et al., 2002).

In one embodiment, the genetic modification is a mutation in an endogenous gene encoding an exporter of a branched chain amino acid. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export branched chain amino acid(s) from the bacterial cell.

Assays for testing the activity of an exporter of a branched chain amino acid, e.g., leucine, are well known to one of ordinary skill in the art. For example, export of a branched chain amino acid, such as leucine, may be determined using the methods described by Haney et al., J. Bact., 174(1):108-15, 1992; Rahmanian et al., J. Bact., 116(3):1258-66, 1973; and Ribardo and Hendrixson, J. Bact., 173(22):6233-43, 2011, the entire contents of which are expressly incorporated herein by reference.

In another embodiment, the genetic modification is a mutation in a promoter of an endogenous gene encoding an exporter of a branched chain amino acid. In one embodiment, the genetic mutation results in decreased expression of the leuE gene. In one embodiment, leuE gene expression is reduced by about 50%, 75%, or 100%. In another embodiment, leuE gene expression is reduced about two-fold, three-fold, four-fold, or five-fold. In another embodiment, the genetic mutation completely inhibits expression of the leuE gene.

Assays for testing the level of expression of a gene, such as an exporter of a branched chain amino acid, e.g., leuE, are well known to one of ordinary skill in the art. For example, reverse-transcriptase polymerase chain reaction may be used to detect the level of mRNA expression of a gene. Alternatively, Western blots using antibodies directed against a protein may be used to determine the level of expression of the protein.

In yet another embodiment, the genetic modification is an overexpression of a repressor of an exporter of a branched chain amino acid. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

D. Disease Associated with Branched Chain Amino Acids

In one embodiment, the disorder involving the catabolism of a branched chain amino acid is a disorder caused by the activation of mTor (mammalian target of rapamycin). mTor is a serine-threonine kinase and has been implicated in a wide range of biological processes including transcription, translation, autophagy, actin organization and ribosome biogenesis, cell growth, cell proliferation, cell motility, and survival. mTOR exists in two complexes, mTORC1 and mTORC2. mTORC1 contains the raptor subunit and mTORC2 contains rictor. These complexes are differentially regulated, and have distinct substrate specificities and rapamycin sensitivity. For example, mTORC1 phosphorylates S6 kinase (S6K) and 4EBP1, promoting increased translation and ribosome biogenesis to facilitate cell growth and cell cycle progression. S6K also acts in a feedback pathway to attenuate PI3K/Akt activation. mTORC2 is generally insensitive to rapamycin and is thought to modulate growth factor signaling by phosphorylating the C-terminal hydrophobic motif of some AGC kinases, such as Akt.

It is known in the art that mTor activation is caused by branched chain amino acids or alpha keto acids in the subject (see, for example, Harlan et al., Cell Metabolism, 17:599-606, 2013). Specifically, activation of mTorC1 (mTor complex 1) is caused by leucine (see Han et al., Cell, 149:410-424, 2012 and Lynch, J. Nutr., 131(3):861S-865S, 2001). Thus, in one embodiment, the disclosure provides methods of treating disorders involving the catabolism of leucine, caused by the activation of mTor by leucine in the subject. In one embodiment, the leucine levels in the subject are normal, and lowering leucine levels in the subject leads to the decreased activity of mTor and, thus, treatment of the disease. In another embodiment, the leucine levels in the subject are increased, and lowering leucine levels in the subject leads to the decreased activity of mTor and, thus, treatment of the disease. In one embodiment, the activation of mTor is increased as compared to the normal level of activation of mTor in a healthy subject, and lowering leucine levels in the subject leads to the decreased activation of mTor and, thus, treatment of the disease. In one embodiment, the level of activity of mTor is increased as compared to the normal level of activity of mTor in a healthy subject, and lowering leucine levels in the subject leads to the decreased activity of mTor and, thus, treatment of the disease. In another embodiment, the expression of mTor is increased as compared to the normal level of expression of mTor in a healthy subject, and lowering leucine levels in the subject leads to the decreased activity of mTor and, thus, treatment of the disease. In one embodiment, the activation of mTor is an abnormal activation of mTor.

Diseases caused by the activation of mTor are known in the art. See, for example, Laplante and Sabatini, Cell, 149(2):74-293, 2012. As used herein, the term “disease caused by the activation of mTor” includes cancer, obesity, type 2 diabetes, neurodegeneration, autism, Alzheimer's disease, Lymphangioleiomyomatosis (LAM), transplant rejection, glycogen storage disease, obesity, tuberous sclerosis, hypertension, cardiovascular disease, hypothalamic activation, musculoskeletal disease, Parkinson's disease, Huntington's disease, psoriasis, rheumatoid arthritis, lupus, multiple sclerosis, Leigh's syndrome, and Friedrich's ataxia.

2. Arginine

A. Arginine Catabolism Enzymes

Arginine catabolism enzymes may be expressed or modified in the bacteria disclosed herein in order to enhance catabolism of arginine. As used herein, the term “arginine catabolism enzyme” refers to an enzyme involved in the catabolism of arginine. Specifically, when an arginine catabolism enzyme is expressed in a recombinant bacterial cell, the bacterial cell catabolizes more arginine when the catabolism enzyme is expressed than unmodified bacteria of the same bacterial subtype under the same conditions. Thus, the genetically engineered bacteria comprising a heterologous gene encoding an arginine catabolism enzyme can catabolize arginine to treat a disease associated with arginine, such as cancer.

In one embodiment, the arginine catabolism enzyme increases the rate of arginine catabolism in the cell. In one embodiment, the arginine catabolism enzyme decreases the level of arginine in the cell. In another embodiment, the arginine catabolism enzyme increases the level of agmatine in the cell.

Arginine catabolism enzymes are well known to those of skill in the art (see, e.g., Giles and Graham (2007) J. Bact. 187(20): 7376-83). In bacteria and plants, arginine decarboxylase enzymes (EC 4.1.1.19) are capable of converting arginine into agmatine and carbon dioxide. For example, Escherichia coli contains two types of arginine decarboxylase: degradative arginine decarboxylase and biosynthetic arginine decarboxylase. The expression of the degradative arginine decarboxylase, AdiA, encoded by the adiA gene, is induced in response to acidic pH, anaerobic conditions, and rich medium (see Stim and Bennett (1993) J. Bact. 175(5): 1221-34). Biosynthetic arginine decarboxylase (ADC; also known as SpeA) is constitutively expressed regardless of pH variations and is involved in the biosynthesis of polyamines (e.g., putrescine, spermidine and spermine) (see Forouhar et al. (2010) Acta Cryst. F66: 1562-6). Both types of arginine decarboxylase mediate the catabolism of arginine by removing acidic carboxyl groups from arginine, and utilize pyroxidal 5′-phosphate as a cofactor (see Stim-Herndon et al. (1996) Microbiology 142: 1311-20).

In some embodiments, an arginine catabolism enzyme is encoded by a gene encoding an arginine catabolism enzyme derived from a bacterial species. In some embodiments, an arginine catabolism enzyme is encoded by a gene encoding an arginine catabolism enzyme derived from a non-bacterial species. In some embodiments, an arginine catabolism enzyme is encoded by a gene derived from a eukaryotic species, e.g., a yeast species or a plant species. In some embodiments, an arginine catabolism enzyme is encoded by a gene derived from a plant species. In one embodiment, the gene encoding the arginine catabolism enzyme is derived from an organism of the genus or species that includes, but is not limited to, Chlamydophila and Escherichia.

In one embodiment, the arginine catabolism enzyme is an arginine decarboxylase (also known as ArgDC). As used herein “arginine decarboxylase” refers to any polypeptide having enzymatic activity that catalyzes the conversion of arginine to agmatine and carbon dioxide. Arginine decarboxylase sequences are available from many microorganism sources, including those disclosed herein. For example, the arginine decarboxylase enzyme AdiA is capable of metabolizing arginine (see, for example, de la Plaza et al., FEMS Microbiol. Lett. 2004, 238(2):367-374), and a cytosolically active KivD should generally exhibit the ability to convert ketoisovalerate to isobutyraldehyde. Some arginine decarboxylase enzymes employ the co-factor pyridoxal 5′-phosphate (PLP).

In one embodiment, the arginine decarboxylase gene is derived from an organism of the genus or species that includes, but is not limited to, Chlamydophila, e.g., Chlamydophila pneumoniae CWL029) (see, e.g., Giles and Graham (2007)), and Escherichia coli.

In one embodiment, the arginine decarboxylase gene is a adiA gene. In another embodiment, the adiA gene is a Escherichia coli adiA gene.

Accordingly, in one embodiment, the adiA gene has at least about 80% identity with the sequence of SEQ ID NO:12. Accordingly, in one embodiment, the adiA gene has at least about 90% identity with the sequence of SEQ ID NO:12. Accordingly, in one embodiment, the adiA gene has at least about 95% identity with the sequence of SEQ ID NO:12. Accordingly, in one embodiment, the adiA gene has at least about 85%, 86%, 87%, 88%, 89%, 90%, 91%, 92%, 93%, 94%, 95%, 96%, 97%, 98%, or 99% identity with the sequence of SEQ ID NO:12. In another embodiment, the adiA gene comprises the sequence of SEQ ID NO:12. In yet another embodiment the adiA gene consists of the sequence of SEQ ID NO:12.

The present disclosure further comprises genes encoding functional fragments of an arginine decarboxylase gene or functional variants of an arginine decarboxylase gene.

Assays for testing the activity of an arginine catabolism enzyme, an arginine catabolism enzyme functional variant, or an arginine catabolism enzyme functional fragment are well known to one of ordinary skill in the art. For example, arginine catabolism can be assessed by expressing the protein, functional variant, or fragment thereof, in a recombinant bacterial cell that lacks endogenous arginine catabolism enzyme activity. Arginine catabolism can be assessed using the arginine decarboxylase assay method (also known as a ¹⁴CO₂ capture assay) (see, e.g., Graham et al. (2002) J. Biol. Chem. 277: 23500-7; or Morris and Boecker (1983) Methods Enzymol. 94: 125-134).

In one embodiment of the disclosure, the gene encoding the arginine catabolism enzyme is an arginine decarboxylase gene. In another embodiment, the gene encoding the arginine decarboxylase is coexpressed with an additional arginine catabolism enzyme, for example, an arginine deiminase enzyme.

Genetic circuits and bacterial strains for the synthesis of arginine are described in PCT/US2015/64140 filed Dec. 4, 2015 and PCT/2016/34200 filed May 25, 2016, both of which applications are hereby incorporated by reference in their entireties, including the drawings.

B. Transporters of Arginine

Arginine transporters may be expressed or modified in the recombinant bacteria described herein in order to enhance arginine transport into the cell. Specifically, when the transporter of arginine is expressed in the recombinant bacterial cells described herein, the bacterial cells import more arginine into the cell when the transporter is expressed than unmodified bacteria of the same bacterial subtype under the same conditions. Thus, the genetically engineered bacteria comprising a heterologous gene encoding transporter of arginine which may be used to import arginine into the bacteria so that any gene encoding an arginine catabolism enzyme expressed in the organism, e.g., co-expressed arginine aminotransferase, can catabolize the arginine to treat a disease associated with amino acid metabolism, such as cancer.

The uptake of arginine into bacterial cells is mediated by proteins well known to those of skill in the art. For example, three different arginine transport systems have been characterized in several bacteria: the arginine-specific system encoded by the artPIQM operon and the artJ gene (see, e.g., Wissenbach et al. (1993) J. Bacteriol. 175(11): 3687-8); the basic amino acid uptake system, known as LAO (lysine, arginine, ornithine) (see, e.g., Rosin et al. (1971) J. Biol. Chem. 246: 3653-62); and the AO (arginine, ornithine) system (see, e.g., Celis (1977) J. Bacteriol. 130: 1234-43). Transport by the arginine-specific system is mediated by several proteins encoded by the two transcriptional units, the artPIQM operon and the artJ gene. In this system, ArtP (encoded by artP) is an ATPase, ArtQ and ArtM (encoded by artQ and artM, respectively) are transmembrane proteins, and ArtI and ArtJ (encoded by artI and artJ, respectively) are arginine-binding periplasmic proteins. This system has been well characterized in Escherichia coli (see, e.g., Wissenbach U. (1995) Mol. Microbiol. 17(4): 675-86; Wissenbach et al. (1993) J. Bacteriol. 175(11): 3687-88). In addition, bacterial systems that are homologous and orthologous of the E. coli arginine-specific system have been characterized in other bacterial species, including, for example, Haemophilus influenzae (see, e.g., Mironov et al. (1999) Nucleic Acids Res. 27(14): 2981-9). The second arginine transport system, the basic amino acid LAO system, consists of the periplasmic LAO protein (also referred to herein as ArgT; encoded by argT), which binds lysine, arginine and ornithine, and the membranous and membrane-associated proteins of the histidine permease (Q M P complex), encoded by the hisJQMP operon, resulting in the uptake of arginine (see, e.g., Oh et al. (1994) J. Biol. Chem. 269(42): 26323-30). Members of the basic amino acid LAO system have been well characterized in Escherichia coli and Salmonella enterica. Finally, the third arginine transport system, the AO system, consists of the binding protein AbpS (encoded by abpS) and the ATP hydrolase ArgK (encoded by argK) which mediate the ATP-dependent uptake of arginine (see, e.g., Celis et al. (1998) J. Bacteriol. 180(18): 4828-33).

In one embodiment, the at least one gene encoding a transporter of arginine is the artJ gene. In one embodiment, the at least one gene encoding a transporter of arginine is the artPIQM operon. In one embodiment, the at least one gene encoding a transporter of arginine is the artP gene. In one embodiment, the at least one gene encoding a transporter of arginine is the artI gene. In one embodiment, the at least one gene encoding a transporter of arginine is the artQ gene. In one embodiment, the at least one gene encoding a transporter of arginine is the artM gene. In one embodiment, the at least one gene encoding a transporter of arginine is the argT gene. In one embodiment, the at least one gene encoding a transporter of arginine is the hisJQMP operon. In one embodiment, the at least one gene encoding a transporter of arginine is the hisJ gene. In one embodiment, the at least one gene encoding a transporter of arginine is the hisQ gene. In one embodiment, the at least one gene encoding a transporter of arginine is the hisM gene. In one embodiment, the at least one gene encoding a transporter of arginine is the hisP gene. In one embodiment, the at least one gene encoding a transporter of arginine is the abpS gene. In one embodiment, the at least one gene encoding a transporter of arginine is the argK gene. In another embodiment, the at least one gene encoding a transporter of arginine comprises the artPIQM operon and the artJ gene. In another embodiment, the at least one gene encoding a transporter of arginine comprises the hisJQMP operon and the argT gene. In yet another embodiment, the at least one gene encoding a transporter of arginine comprises the abpS gene and the argK gene.

In one embodiment, the argT gene has at least about 80% identity with the sequence of SEQ ID NO:13. Accordingly, in one embodiment, the argT gene has at least about 90% identity with the sequence of SEQ ID NO:13. Accordingly, in one embodiment, the argT gene has at least about 95% identity with the sequence of SEQ ID NO:13. Accordingly, in one embodiment, the argT gene has at least about 85%, 86%, 87%, 88%, 89%, 90%, 91%, 92%, 93%, 94%, 95%, 96%, 97%, 98%, or 99% identity with the sequence of SEQ ID NO:13. In another embodiment, the argT gene comprises the sequence of SEQ ID NO:13. In yet another embodiment the argT gene consists of the sequence of SEQ ID NO:13.

In one embodiment, the artP gene has at least about 80% identity with the sequence of SEQ ID NO:14. Accordingly, in one embodiment, the artP gene has at least about 90% identity with the sequence of SEQ ID NO:14. Accordingly, in one embodiment, the artP gene has at least about 95% identity with the sequence of SEQ ID NO:14. Accordingly, in one embodiment, the artP gene has at least about 85%, 86%, 87%, 88%, 89%, 90%, 91%, 92%, 93%, 94%, 95%, 96%, 97%, 98%, or 99% identity with the sequence of SEQ ID NO:14. In another embodiment, the artP gene comprises the sequence of SEQ ID NO:14. In yet another embodiment the artP gene consists of the sequence of SEQ ID NO:14.

In one embodiment, the artI gene has at least about 80% identity with the sequence of SEQ ID NO:15. Accordingly, in one embodiment, the artI gene has at least about 90% identity with the sequence of SEQ ID NO:15. Accordingly, in one embodiment, the artI gene has at least about 95% identity with the sequence of SEQ ID NO:15. Accordingly, in one embodiment, the artI gene has at least about 85%, 86%, 87%, 88%, 89%, 90%, 91%, 92%, 93%, 94%, 95%, 96%, 97%, 98%, or 99% identity with the sequence of SEQ ID NO:15. In another embodiment, the artI gene comprises the sequence of SEQ ID NO:15. In yet another embodiment the artI gene consists of the sequence of SEQ ID NO:15.

In one embodiment, the artQ gene has at least about 80% identity with the sequence of SEQ ID NO:16. Accordingly, in one embodiment, the artQ gene has at least about 90% identity with the sequence of SEQ ID NO:16. Accordingly, in one embodiment, the artQ gene has at least about 95% identity with the sequence of SEQ ID NO:16. Accordingly, in one embodiment, the artQ gene has at least about 85%, 86%, 87%, 88%, 89%, 90%, 91%, 92%, 93%, 94%, 95%, 96%, 97%, 98%, or 99% identity with the sequence of SEQ ID NO:16. In another embodiment, the artQ gene comprises the sequence of SEQ ID NO:16. In yet another embodiment the artQ gene consists of the sequence of SEQ ID NO:16.

In one embodiment, the artM gene has at least about 80% identity with the sequence of SEQ ID NO:17. Accordingly, in one embodiment, the artM gene has at least about 90% identity with the sequence of SEQ ID NO:17. Accordingly, in one embodiment, the artM gene has at least about 95% identity with the sequence of SEQ ID NO:17. Accordingly, in one embodiment, the artM gene has at least about 85%, 86%, 87%, 88%, 89%, 90%, 91%, 92%, 93%, 94%, 95%, 96%, 97%, 98%, or 99% identity with the sequence of SEQ ID NO:17. In another embodiment, the artM gene comprises the sequence of SEQ ID NO:17. In yet another embodiment the artM gene consists of the sequence of SEQ ID NO:17.

In one embodiment, the artJ gene has at least about 80% identity with the sequence of SEQ ID NO:18. Accordingly, in one embodiment, the artJ gene has at least about 90% identity with the sequence of SEQ ID NO:18. Accordingly, in one embodiment, the artJ gene has at least about 95% identity with the sequence of SEQ ID NO:18. Accordingly, in one embodiment, the artJ gene has at least about 85%, 86%, 87%, 88%, 89%, 90%, 91%, 92%, 93%, 94%, 95%, 96%, 97%, 98%, or 99% identity with the sequence of SEQ ID NO:18. In another embodiment, the artJ gene comprises the sequence of SEQ ID NO:18. In yet another embodiment the artJ gene consists of the sequence of SEQ ID NO:18.

In some embodiments, the transporter of arginine is encoded by a transporter of arginine gene derived from a bacterial genus or species, including but not limited to, Escherichia, Haemophilus, Salmonella, Escherichia coli, Haemophilus influenza, Salmonella enterica, or Salmonella typhimurium. In some embodiments, the bacterial species is Escherichia coli. In some embodiments, the bacterial species is Escherichia coli strain Nissle.

Assays for testing the activity of a transporter of arginine, a functional variant of a transporter of arginine, or a functional fragment of transporter of arginine are well known to one of ordinary skill in the art. For example, import of arginine may be determined using the methods as described in Sakanaka et al (2015) J. Biol. Chem. 290(35): 21185-98, the entire contents of each of which are expressly incorporated by reference herein.

In one embodiment, when the transporter of an arginine is expressed in the recombinant bacterial cells described herein, the bacterial cells import 10% more arginine into the bacterial cell when the transporter is expressed than unmodified bacteria of the same bacterial subtype under the same conditions. In another embodiment, when the transporter of arginine is expressed in the recombinant bacterial cells described herein, the bacterial cells import 20%, 30%, 40%, 50%, 60%, 70%, 80%, 90% or 100% more arginine into the bacterial cell when the transporter is expressed than unmodified bacteria of the same bacterial subtype under the same conditions. In yet another embodiment, when the transporter of arginine is expressed in the recombinant bacterial cells described herein, the bacterial cells import two-fold more arginine into the cell when the transporter is expressed than unmodified bacteria of the same bacterial subtype under the same conditions. In yet another embodiment, when the transporter of arginine is expressed in the recombinant bacterial cells described herein, the bacterial cells import three-fold, four-fold, five-fold, six-fold, seven-fold, eight-fold, nine-fold, or ten-fold more arginine into the cell when the transporter is expressed than unmodified bacteria of the same bacterial subtype under the same conditions.

C. Exporters of Arginine

Arginine exporters may be modified in the recombinant bacteria described herein in order to reduce arginine export from the cell. Specifically, when the recombinant bacterial cells described herein comprise a genetic modification that reduces export of arginine, the bacterial cells retain more arginine in the bacterial cell than unmodified bacteria of the same bacterial subtype under the same conditions. Thus, the recombinant bacteria comprising a genetic modification that reduces export of arginine may be used to retain more arginine in the bacterial cell so that any arginine catabolism enzyme expressed in the organism, e.g., co-expressed arginine aminotransferase, can catabolize the arginine.

The export of arginine from bacterial cells is mediated by proteins well known to those of skill in the art. For example, the arginine exporter ArgO has been characterized in Escherichia coli (Pathania and Sardesai (2015) J. Bacteriol. 197(12): 2036-47; incorporated herein by reference). ArgO is encoded by the argO gene in Escherichia coli (also known as yeaS). In addition, an ortholog of ArgO, LysE, mediates the export of both arginine and lysine in Corynebacterium glutamicum (Bellmann et al. (2001) Microbiology 147: 1765-74).

In one embodiment, the genetic modification is a mutation in an endogenous gene encoding an exporter of arginine. In one embodiment, the recombinant bacterial cell comprises a genetic modification that reduces export of arginine from the bacterial cell, wherein the endogenous gene encoding an exporter of arginine is an argO gene. In another embodiment, the endogenous gene encoding an exporter of arginine is a lysE gene. In one embodiment, the recombinant bacterial cell comprises a genetic modification that reduces export of arginine from the bacterial cell and a heterologous gene encoding an arginine catabolism enzyme. In one embodiment, the recombinant bacteria further comprise a heterologous gene encoding a transporter of arginine.

In one embodiment, the genetic modification reduces export of arginine from the bacterial cell. In one embodiment, the bacterial cell is from a bacterial genus or species that includes but is not limited to, Escherichia coli and Corynebacterium glutamicum. In another embodiment, the bacterial cell is an Escherichia coli bacterial cell. In another embodiment, the bacterial cell is an Escherichia coli strain Nissle bacterial cell.

In one embodiment, the argO gene has at least about 80% identity with the sequence of SEQ ID NO:19. Accordingly, in one embodiment, the argO gene has at least about 90% identity with the sequence of SEQ ID NO:19. Accordingly, in one embodiment, the argO gene has at least about 95% identity with the sequence of SEQ ID NO:19. Accordingly, in one embodiment, the argO gene has at least about 85%, 86%, 87%, 88%, 89%, 90%, 91%, 92%, 93%, 94%, 95%, 96%, 97%, 98%, or 99% identity with the sequence of SEQ ID NO:19. In another embodiment, the argO gene comprises the sequence of SEQ ID NO:19. In yet another embodiment the argO gene consists of the sequence of SEQ ID NO:19.

In one embodiment, the genetic mutation results in an exporter having reduced activity as compared to a wild-type exporter protein. In another embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export arginine from the bacterial cell. Assays for testing the activity of an exporter of an arginine are well known to one of ordinary skill in the art.

In another embodiment, the genetic modification is a mutation in a promoter of an endogenous gene encoding an exporter of arginine.

Assays for testing the activity of an exporter of arginine are well known to one of ordinary skill in the art. For example, export of arginine may be determined using the methods described by Bellmann et al. (2001) Microbiology 147: 1765-74), the entire contents of which are expressly incorporated herein by reference.

In yet another embodiment, the genetic modification is an overexpression of a repressor of an exporter of arginine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

3. Lysine

A. Lysine Catabolism Enzymes

Lysine catabolism enzymes may be expressed or modified in the bacteria disclosed herein in order to enhance catabolism of lysine. As used herein, the term “lysine catabolism enzyme” refers to an enzyme involved in the catabolism of lysine. Specifically, when a lysine catabolism enzyme is expressed in a recombinant bacterial cell, the bacterial cell catabolizes more lysine when the catabolism enzyme is expressed than unmodified bacteria of the same bacterial subtype under the same conditions. Thus, the genetically engineered bacteria comprising a heterologous gene encoding a lysine catabolism enzyme can catabolize lysine to treat a disease associated with lysine, such as cancer.

In one embodiment, the lysine catabolism enzyme increases the rate of lysine catabolism in the cell. In one embodiment, the lysine catabolism enzyme decreases the level of lysine in the cell. In another embodiment, the lysine catabolism enzyme increases the level of glutamate in the cell. In one embodiment, the lysine catabolism enzyme increases the level of α-aminoadipic acid in the cell. In another embodiment, the lysine catabolism enzyme increases the level of saccharopine in the cell. In yet another embodiment, the lysine catabolism enzyme increases the level of α-aminoadipic-δ-semialdehyde in the cell. In one embodiment, the lysine catabolism enzyme increases the level of 2-aminoadipate 6-semialdehyde in the cell. In another embodiment, the lysine catabolism enzyme increases the level of 1,2-didehydropiperidine-2-carboxylate in the cell. In yet another embodiment, the lysine catabolism enzyme increases the level of _{D}-lysine in the cell. In one embodiment, the lysine catabolism enzyme increases the level of Δ¹-piperidine-2-carboxylate in the cell. In another embodiment, the lysine catabolism increases the level of pipecolate in the cell.

Lysine catabolism enzymes are well known to those of skill in the art (see, e.g., Neshich et al. (2013) ISME J. 7(12): 2400-10), and several lysine catabolism pathways have been identified and characterized in prokaryotes and eukaryotes. For example, four lysine catabolism pathways have been characterized in prokaryotes. In the saccharopine pathway, lysine is converted to α-aminoadipic semialdehyde via a two-step reaction in which lysine-ketoglutarate reductase condenses lysing and α-ketoglutarate into saccharopine, and saccharopine dehydrogenase hydrolyzes saccharopine into α-aminoadipic semialdehyde and glutamate. The second pathway involves the oxidative deamination of lysine as mediated by lysine dehydrogenase. In the third pathway, lysine aminotransferase catalyzes the transamination of α-ketoglutarate, yielding α-aminoadipic semialdehyde and glutamate. Finally, in the fourth pathway, a multistep catabolic reaction commences with the conversion of L-lysine into _{D}-lysine by lysine racemase. _{D}-lysine is then deaminated by an aminotransferase (i.e., _{D}-lysine aminotransferase to form Δ¹-piperidine-2-carboxylate, which is then converted to pipecolate by Δ¹-piperidine-2-carboxylate reductase. Pipecolate is then oxidized to α-aminoadipic semialdehyde by pipecolate oxidase.

In some embodiments, a lysine catabolism enzyme is encoded by a gene encoding a lysine catabolism enzyme derived from a bacterial species. In some embodiments, a lysine catabolism enzyme is encoded by a gene encoding a lysine catabolism enzyme derived from a non-bacterial species. In some embodiments, a lysine catabolism enzyme is encoded by a gene derived from a eukaryotic species, e.g., a yeast species or a plant species. In some embodiments, a lysine catabolism enzyme is encoded by a gene derived from a plant species. In one embodiment, the gene encoding the lysine catabolism enzyme is derived from an organism of the genus or species that includes, but is not limited to, Actinosynnema (e.g., Actinosynnema mirum), Agrobacterium (e.g., Agrobacterium tumefaciens), Alcaligenes (e.g., Alcaligenes eutropha H16) Anoxybacillus (e.g., Anoxybacillus flavithermus), Arabidopsis (e.g. Arabidopsis thaliana), Bacillus (e.g., Bacillus methanolicus), Brachypodium, Bradyrhizobium (e.g., Bradyrhizobium sp. BTAi1), Candidatus Nitrospira (e.g., Candidatus Nitrospira defluvii), Comamonas (e.g., Comamonas testosterone), Cupriavidus (e.g., Cupriavidus metallidurans and Cupriavidus necator), Desulfotalea (e.g., Desulfotalea psychrophila), Flavobacterium, (e.g., Flavobacterium limnosediminis and Flavobacterium sp. EM1321), Francisella (e.g., Francisella novicida and Francisella philomiragia), Frankia (e.g., Framkia alni), Geobacillus (e.g., Geobacillus kaustophilus, Geobacillus stearothermophilus and Geobacillus thermodenitrificans), Kangiella (e.g., Kangiella koreensis), Legionella (e.g., Legionella longbeachae and Legionella pneumophila), Leptothrix (e.g., Leptothrix cholodnii), Medicago, Mycobacterium (e.g., Mycobacterium abscessus, Mycobacterium africanum, Mycobacterium avium, Mycobacterium bovis, Mycobacterium canetti, and Mycobacterium tuberculosis), Nicotiana (e.g., Nicotiana tabacum) Nocardia (e.g., Nocardia farcinica), Oceanobacillus (e.g., Oceanobacillus iheyensis), Oryza (e.g., Oryza sativa), Poplar (e.g., Poplar trychocarpa), Populus (Populus nigra, Populus tremula, and Populus trichocarpa), Proteus (e.g., Proteus vulgaris), Pseudomonas (e.g., Pseudomonas putida), Pyrococcus (e.g., Pyrococcus horikoshii), Roseobacter (e.g., Roseobacter sp. MED193), Roseovarius (e.g., Roseovarius sp. 217), Rhodococcus (e.g., Rhodococcus equi, and Rhodococcus erythropolis), Saccharomyces (e.g., Saccharomyces cerevisiae), Salinispora (e.g., Salinispora arenicola), Silicibacter (e.g., Silicibacter pomeroyi), Sorghum (e.g., Sorghum bicolor), Streptomyces (e.g., Streptomyces scabies), Triticum (e.g., Triticum tugidum), Vitis (e.g., Vitis vinifera), Weeksella (e.g., Weeksella virosa), and Zea (e.g., Zea mays).

Lysine Ketoglutarate Reductase and Saccharopine Dehydrogenase Enzymes

In one embodiment, the lysine catabolism enzyme is a lysine ketoglutarate reductase (LKR; E.C. 1.5.1.8). As used herein, “lysine ketoglutarate reductase” refers to any polypeptide having enzymatic activity that catalyzes the condensation of lysine and α-ketoglutarate into saccharopine. In one embodiment, the lysine catabolism enzyme is a saccharopine dehydrogenase (SDH; E.C. 1.5.1.9). As used herein, “saccharopine dehydrogenase” refers to any polypeptide that catalyzes the hydrolysis of saccharopine into α-aminoadipic semialdehyde (AAAS). In some embodiments, the lysine catabolism enzyme is a catabolic bifunctional enzyme lysine ketoglutarate reductase—saccharopine dehydrogenase (LKR/SDH). For example, in some plants and animals, the LKR/SDH gene encodes an open reading frame composed of fused LKR and SDH domains, whereas in some yeast and fungi, the LKR and SDH activities are encoded by separate genes (see, e.g., Anderson et al. (2010) BMC Plant Biology 10: 113). Lysine ketoglutarate reductase enzymes, saccharopine dehydrogenase, and bifunctional lysine ketoglutarate reductase—saccharopine dehydrogenase enzymes have been characterized from many organisms (see, e.g., Anderson et al. (2010); and Neshich et al. (2013)).

In one embodiment, the lysine ketoglutarate reductase gene, the saccharopine dehydrogenase gene, or the bifunctional enzyme lysine ketoglutarate reductase—saccharopine dehydrogenase gene is derived from an organism of the genus or species that includes, but is not limited to, Actinosynnema, Arabidopsis, Brachypodium, Medicago, Nicotiana, Oryza, Poplar, Populus, Roseobacter, Roseovarius, Salinispora, Silicibacter, Sorghum, Triticum, Vitis, and Zea, Actinosynnema mirum, Arabidopsis thaliana, Nicotiana tabacum, Oryza sativa, Poplar trychocarpa, Populus nigra, Populus tremula, Populus trichocarpa, Roseobacter sp. MED193, Roseovarius sp. 217, Salinispora arenicola, Silicibacter pomeroyi, Sorghum bicolor, Triticum tugidum, Vitis vinifera, and Zea mays.

In one embodiment, the lysine ketoglutarate reductase gene has at least about 80% identity with the sequence of SEQ ID NO:20. Accordingly, in one embodiment, the lysine ketoglutarate reductase gene has at least about 90% identity with the sequence of SEQ ID NO:20. Accordingly, in one embodiment, the lysine ketoglutarate reductase gene has at least about 95% identity with the sequence of SEQ ID NO:20. Accordingly, in one embodiment, the lysine ketoglutarate reductase gene has at least about 85%, 86%, 87%, 88%, 89%, 90%, 91%, 92%, 93%, 94%, 95%, 96%, 97%, 98%, or 99% identity with the sequence of SEQ ID NO:20. In another embodiment, the lysine ketoglutarate reductase gene comprises the sequence of SEQ ID NO:20. In yet another embodiment the lysine ketoglutarate reductase gene consists of the sequence of SEQ ID NO:20.

In one embodiment, the saccharopine dehydrogenase gene has at least about 80% identity with the sequence of SEQ ID NO:21. Accordingly, in one embodiment, the saccharopine dehydrogenase gene has at least about 90% identity with the sequence of SEQ ID NO:21. Accordingly, in one embodiment, the saccharopine dehydrogenase gene has at least about 95% identity with the sequence of SEQ ID NO:21. Accordingly, in one embodiment, the saccharopine dehydrogenase gene has at least about 85%, 86%, 87%, 88%, 89%, 90%, 91%, 92%, 93%, 94%, 95%, 96%, 97%, 98%, or 99% identity with the sequence of SEQ ID NO:21. In another embodiment, the saccharopine dehydrogenase gene comprises the sequence of SEQ ID NO:21. In yet another embodiment the saccharopine dehydrogenase gene consists of the sequence of SEQ ID NO:21.

The present disclosure further comprises genes encoding functional fragments of a lysine ketoglutarate reductase gene or functional variants of a lysine ketoglutarate reductase gene.

The present disclosure also comprises genes encoding functional fragments of an saccharopine dehydrogenase gene or functional variants of an saccharopine dehydrogenase gene.

Assays for testing the activity of a lysine ketoglutarate reductase enzyme, a lysine ketoglutarate reductase enzyme functional variant, or a lysine ketoglutarate reductase enzyme functional fragment are well known to one of ordinary skill in the art. For example, lysine ketoglutarate reductase activity can be assessed by expressing the protein, functional variant, or fragment thereof, in a recombinant bacterial cell that lacks endogenous lysine ketoglutarate reductase enzyme activity. Lysine ketoglutarate reductase activity can then be assessed as described, e.g., in Zhu et al. (2000) Biochem. J. (2000) 351, 215-20, the entire contents of which are incorporated by reference.

Assays for testing the activity of a saccharopine dehydrogenase enzyme, a saccharopine dehydrogenase enzyme functional variant, or a saccharopine dehydrogenase enzyme functional fragment are well known to one of ordinary skill in the art. For example, saccharopine dehydrogenase activity can be assessed by expressing the protein, functional variant, or fragment thereof, in a recombinant bacterial cell that lacks endogenous saccharopine dehydrogenase enzyme activity. Saccharopine dehydrogenase activity can then be assessed as described, e.g., in Zhu et al. (2000) Plant Physiol. 124(3): 1363-72, the entire contents of which are incorporated by reference.

In another embodiment, the gene encoding the lysine ketoglutarate reductase enzyme is co-expressed with a gene encoding a saccharopine dehydrogenase enzyme.

Lysine Aminotransferase Enzymes

In one embodiment, the lysine catabolism enzyme is a lysine aminotransferase (LAT; E.C. 2.6.1.36). As used herein, “lysine aminotransferase” refers to any polypeptide having enzymatic activity that catalyzes the transamination of α-ketoglutarate yielding α-aminoadipic semialdehyde and glutamate. Multiple lysine aminotransferase enzymes are known in the art (see, e.g., Wu et al. (2007) J. Agric. Food Chem. 55(5): 1767-72; Tripathi and Ramanchandran (2006) Acta Crystallogr. Sect. F Struct. Biol. Cryst. Commun. 62(Pt 6): 572-5; and Neshich et al. (2013)).

In one embodiment, the lysine aminotransferase gene is derived from an organism of the genus or species that includes, but is not limited to, Bacillus, Bacillus methanolicus, Desulfotalea, Desulfotalea psychrophila, Frankia, Frankia alni, Mycobacterium, Mycobacterium abscessus, Mycobacterium africanum, Mycobacterium avium, Mycobacterium bovis, Mycobacterium canetti, Mycobacterium tuberculosis, Nocardia, Nocardia farcinica, Rhodococcus, Rhodococcus equi, Rhodococcus erythropolis, Streptomyces, Streptomyces clavuligerus, Weeksella, and Weeksella virosa.

In one embodiment, the lysine aminotransferase gene has at least about 80% identity with the sequence of SEQ ID NO:22. Accordingly, in one embodiment, the lysine aminotransferase gene has at least about 90% identity with the sequence of SEQ ID NO:22. Accordingly, in one embodiment, the lysine aminotransferase gene has at least about 95% identity with the sequence of SEQ ID NO:22. Accordingly, in one embodiment, the lysine aminotransferase gene has at least about 85%, 86%, 87%, 88%, 89%, 90%, 91%, 92%, 93%, 94%, 95%, 96%, 97%, 98%, or 99% identity with the sequence of SEQ ID NO:22. In another embodiment, the lysine aminotransferase gene comprises the sequence of SEQ ID NO:22. In yet another embodiment the lysine aminotransferase gene consists of the sequence of SEQ ID NO:22.

The present disclosure further comprises genes encoding functional fragments of a lysine aminotransferase gene or functional variants of a lysine aminotransferase gene.

Assays for testing the activity of a lysine aminotransferase enzyme, a lysine aminotransferase enzyme functional variant, or a lysine aminotransferase enzyme functional fragment are well known to one of ordinary skill in the art. For example, lysine aminotransferase activity can be assessed by expressing the protein, functional variant, or fragment thereof, in a recombinant bacterial cell that lacks endogenous lysine aminotransferase enzyme activity. Lysine aminotransferase activity can then be assessed as described, e.g., in Namwat et al. (2002) J. Bacteriol. 184(17): 4811-8, the entire contents of which are incorporated by reference.

Lysine Dehydrogenase Enzymes

In one embodiment, the lysine catabolism enzyme is a lysine dehydrogenase (LysDH; E.C. 1.4.1.15). As used herein, “lysine dehydrogenase” refers to any polypeptide having enzymatic activity that catalyzes the oxidative deamination of lysine yielding 1,2-didehydropiperidine-2-carboxylate (Δ¹-piperideine-2-carboxylate). Multiple lysine dehydrogenase enzymes are known in the art (see, e.g., Misono and Nagasaki (1982) J. Bacteriol. 150(1): 398-401; Misono and Nagasaki (1983) Agric. Biol. Chem. 47: 631-633; Neshich et al. (2013)).

In one embodiment, the lysine dehydrogenase gene is derived from an organism of the genus or species that includes, but is not limited to, Agrobacterium, Agrobacterium tumefaciens, Enterobacter, Enterobacter aerogenes, Micrococcus, Micrococcus flavus, Alcaligenes, Alcaligenes eutropha H16, Anoxybacillus, Anoxybacillus flavithermus, Bacillus, Bacillus sphaericus, Bradyrhizobium, Bradyrhizobium sp. BTAi1, Candidatus Nitrospira, Candidatus Nitrospira defluvii, Comamonas, Comamonas testosterone, Cupriavidus, Cupriavidus metallidurans, Cupriavidus necator, Francisella, Francisella novicida, Francisella philomiragia, Geobacillus, Geobacillus kaustophilus, Geobacillus thermodenitrificans, Kangiella, Kangiella koreensis, Legionella, Legionella longbeachae, Legionella pneumophila, Leptothrix, Leptothrix cholodnii, Oceanobacillus, Oceanobacillus iheyensis, Pedobacter, Pedobacter heparinus, Pyrococcus, Pyrococcus horikoshii, Silicibacter, Silicibacter pomeroyi, Thauera. and Thauera sp. MZ1T.

In one embodiment, the lysine dehydrogenase gene has at least about 80% identity with the sequence of SEQ ID NO:23. Accordingly, in one embodiment, the lysine dehydrogenase gene has at least about 90% identity with the sequence of SEQ ID NO:23. Accordingly, in one embodiment, the lysine dehydrogenase gene has at least about 95% identity with the sequence of SEQ ID NO:23. Accordingly, in one embodiment, the lysine dehydrogenase gene has at least about 85%, 86%, 87%, 88%, 89%, 90%, 91%, 92%, 93%, 94%, 95%, 96%, 97%, 98%, or 99% identity with the sequence of SEQ ID NO:23. In another embodiment, the lysine dehydrogenase gene comprises the sequence of SEQ ID NO:23. In yet another embodiment the lysine dehydrogenase gene consists of the sequence of SEQ ID NO:23.

The present disclosure further comprises genes encoding functional fragments of a lysine dehydrogenase gene or functional variants of a lysine dehydrogenase gene.

Assays for testing the activity of a lysine dehydrogenase enzyme, a lysine dehydrogenase enzyme functional variant, or a lysine dehydrogenase enzyme functional fragment are well known to one of ordinary skill in the art. For example, lysine dehydrogenase activity can be assessed by expressing the protein, functional variant, or fragment thereof, in a recombinant bacterial cell that lacks endogenous lysine dehydrogenase enzyme activity. Lysine dehydrogenase activity can then be assessed by HPLC as described, e.g., in Yoneda et al. (2010) J Biol. Chem. 285(11): 8444-53, the entire contents of which are incorporated by reference.

Lysine Racemase, _{D}-Lysine Aminotransferase and Δ¹-piperidine-2-carboxylate Reductase Enzymes

In one embodiment, the lysine catabolism enzyme is a lysine racemase (E.C. 5.1.1.5). As used herein, “lysine racemase” refers to any polypeptide having enzymatic activity that catalyzes the conversion of _{L}-lysine into _{D}-lysine. In one embodiment, the lysine catabolism enzyme is a _{D}-lysine aminotransferase. As used herein, “_{D}-lysine aminotransferase” refers to any polypeptide that catalyzes the deamination of D-lysine to form Δ¹-piperidine-2-carboxylate. In some embodiments, the lysine catabolism enzyme is a Δ¹-piperidine-2-carboxylate reductase. As used herein, “Δ¹-piperidine-2-carboxylate reductase” refers to any polypeptide that catalyzes the conversion of Δ¹-piperidine-2-carboxylate into pipecolate. Lysine racemase, _{D}-lysine aminotransferase and Δ¹-piperidine-2-carboxylate reductase enzymes are well known in the art (see, e.g., Revelles et al. (2007) J. Bacteriol. 189: 2787-92; Chen et al. (2009) Appl. Environ. Microbiol. 75(15): 5161-5166; Huan and Davisson (1958) J. Biol. Chem. 76: 495-98; and Neshich et al. (2013)).

In one embodiment, the lysine racemase enzyme gene, the _{D}-lysine aminotransferase enzyme gene, or the Δ¹-piperidine-2-carboxylate reductase enzyme gene is derived from an organism of the genus or species that includes, but is not limited to, Proteus, Proteus vulgaris, Pseudomonas, Pseudomonas aeruginosa, and Pseudomonas putida.

In one embodiment, the lysine racemase gene has at least about 80% identity with the sequence of SEQ ID NO:24. Accordingly, in one embodiment, the lysine racemase gene has at least about 90% identity with the sequence of SEQ ID NO:24. Accordingly, in one embodiment, the lysine racemase gene has at least about 95% identity with the sequence of SEQ ID NO:24. Accordingly, in one embodiment, the lysine racemase gene has at least about 85%, 86%, 87%, 88%, 89%, 90%, 91%, 92%, 93%, 94%, 95%, 96%, 97%, 98%, or 99% identity with the sequence of SEQ ID NO:24. In another embodiment, the lysine racemase gene comprises the sequence of SEQ ID NO:24. In yet another embodiment the lysine racemase gene consists of the sequence of SEQ ID NO:24.

The present disclosure further comprises genes encoding functional fragments of a lysine racemase gene or functional variants of a lysine racemase gene.

The present disclosure also comprises genes encoding functional fragments of a _{D}-lysine aminotransferase gene or functional variants of a _{D}-lysine aminotransferase gene.

The present disclosure also comprises genes encoding functional fragments of a Δ¹-piperidine-2-carboxylate reductase gene or functional variants of a Δ¹-piperidine-2-carboxylate reductase gene.

Assays for testing the activity of a lysine racemase enzyme, a lysine racemase enzyme functional variant, or a lysine racemase enzyme functional fragment are well known to one of ordinary skill in the art. For example, lysine racemase activity can be assessed by expressing the protein, functional variant, or fragment thereof, in a recombinant bacterial cell that lacks endogenous lysine racemase enzyme activity. Lysine racemase activity can then be assessed by measuring the production of _{D}-lysine from _{L}-lysine or viceversa by enantioselective column chromatography as described, e.g., in Chen et al. (2009) Appl. Environ. Microbiol. 75(15): 5161-5166, the entire contents of which are incorporated by reference.

Assays for testing the activity of a _{D}-lysine aminotransferase enzyme, a _{D}-lysine aminotransferase enzyme functional variant, or a _{D}-lysine aminotransferase enzyme functional fragment are well known to one of ordinary skill in the art. For example, _{D}-lysine aminotransferase activity can be assessed by expressing the protein, functional variant, or fragment thereof, in a recombinant bacterial cell that lacks endogenous _{D}-lysine aminotransferase enzyme activity. _{D}-lysine aminotransferase activity can then be assessed as described, e.g., in Revelles et al. (2007) J. Bacteriol. 189: 2787-92, the entire contents of which are incorporated by reference.

Assays for testing the activity of a Δ¹-piperidine-2-carboxylate reductase enzyme, a Δ¹-piperidine-2-carboxylate reductase enzyme functional variant, or a Δ¹-piperidine-2-carboxylate reductase enzyme functional fragment are well known to one of ordinary skill in the art. For example, Δ¹-piperidine-2-carboxylate reductase activity can be assessed by expressing the protein, functional variant, or fragment thereof, in a recombinant bacterial cell that lacks endogenous Δ¹-piperidine-2-carboxylate reductase enzyme activity. Δ¹-piperidine-2-carboxylate reductase activity can then be assessed as described, e.g., in Muramatsu et al. (2005) J. Biol. Chem. 2005 280(7): 5329-35, the entire contents of which are incorporated by reference.

In one embodiment, the gene encoding a lysine racemase enzyme is co-expressed with a gene encoding a _{D}-lysine aminotransferase enzyme. In another embodiment, the gene encoding a lysine racemase enzyme is co-expressed with a gene encoding a Δ¹-piperidine-2-carboxylate reductase enzyme. In yet another embodiment, the gene encoding a _{D}-lysine aminotransferase enzyme is co-expressed with a gene encoding a Δ¹-piperidine-2-carboxylate reductase enzyme. In yet an additional embodiment, the gene encoding a lysine racemase enzyme is co-expressed with a gene encoding a _{D}-lysine aminotransferase enzyme and with a gene encoding a Δ¹-piperidine-2-carboxylate reductase enzyme.

In one embodiment, the bacterial cell comprises a heterologous gene encoding a lysine catabolism enzyme. In one embodiment, the bacterial cell comprises a heterologous gene encoding a transporter of lysine and a heterologous gene encoding a lysine catabolism enzyme. In one embodiment, the bacterial cell comprises a heterologous gene encoding a lysine catabolism enzyme and a genetic modification that reduces export of lysine. In one embodiment, the bacterial cell comprises a heterologous gene encoding a transporter of lysine, a heterologous gene encoding a lysine catabolism enzyme, and a genetic modification that reduces export of lysine. Transporters and exporters of lysine are described in more detail in the subsections, below.

B. Transporters of Lysine

Lysine transporters may be expressed or modified in the recombinant bacteria described herein in order to enhance lysine transport into the cell. Specifically, when the transporter of lysine is expressed in the recombinant bacterial cells described herein, the bacterial cells import more lysine into the cell when the transporter is expressed than unmodified bacteria of the same bacterial subtype under the same conditions. Thus, the genetically engineered bacteria comprising a heterologous gene encoding transporter of lysine which may be used to import lysine into the bacteria so that any gene encoding a lysine catabolism enzyme expressed in the organism, e.g., co-expressed lysine aminotransferase, can catabolize the lysine to treat a disease, such as cancer.

The uptake of lysine into bacterial cells is mediated by proteins well known to those of skill in the art. For example, LysP is a lysine-specific permease originally identified in E. coli, that has now been further characterized in other bacterial species (Steffes et al. (1992) J. Bacteriol. 174: 3242-9; Trip et al. (2013) J. Bacteriol. 195(2): 340-50; Nji et al. (2014) Acta Crystallogr. F Struct. Biol. Commun. 70(Pt 10): 1362-7). Another lysine transporter, YsvH, has been described in Bacillus, having similarities to the lysine permease LysI of Corynebacterium glutamicum (Rodionov et al. (2003) Nucleic Acids Res. 31(23): 6748-57).

In one embodiment, the at least one gene encoding a transporter of lysine is the lysP gene. In one embodiment, the bacterial cell described herein has been genetically engineered to comprise at least one heterologous lysP gene. In one embodiment, the at least one gene encoding a transporter of lysine is the Escherichia coli lysP gene. In one embodiment, the at least one gene encoding a transporter of lysine is the Lactococcus lactis lysP gene. In one embodiment, the at least one gene encoding a transporter of lysine is the Pseudomonas aeruginosa lysP gene. In one embodiment, the at least one gene encoding a transporter of lysine is the Klebsiella pneumoniae lysP gene.

In one embodiment, the lysP gene has at least about 80% identity with the sequence of SEQ ID NO:26. Accordingly, in one embodiment, the lysP gene has at least about 90% identity with the sequence of SEQ ID NO:26. Accordingly, in one embodiment, the lysP gene has at least about 95% identity with the sequence of SEQ ID NO:26. Accordingly, in one embodiment, the lysP gene has at least about 85%, 86%, 87%, 88%, 89%, 90%, 91%, 92%, 93%, 94%, 95%, 96%, 97%, 98%, or 99% identity with the sequence of SEQ ID NO:26. In another embodiment, the lysP gene comprises the sequence of SEQ ID NO:26. In yet another embodiment the lysP gene consists of the sequence of SEQ ID NO:26.

In one embodiment, the at least one gene encoding a transporter of lysine is the ysvH gene. In one embodiment, the bacterial cell described herein has been genetically engineered to comprise at least one heterologous ysvH gene. In one embodiment, the at least one gene encoding a transporter of lysine is the Bacillus subtilis ysvH gene. In one embodiment, the at least one gene encoding a transporter of lysine is the Bacillus cereus ysvH gene. In one embodiment, the at least one gene encoding a transporter of lysine is the Bacillus stearothermophilus ysvH gene.

In one embodiment, the at least one gene encoding a transporter of lysine is the Corynebacterium glutamicum (see, e.g., Seep-Feldhaus et al. (1991) Mol. Microbiol. 5(12): 2995-3005, the entire contents of which are incorporated herein by reference).

In one embodiment, the ysvH gene has at least about 80% identity with the sequence of SEQ ID NO:25. Accordingly, in one embodiment, the ysvH gene has at least about 90% identity with the sequence of SEQ ID NO:25. Accordingly, in one embodiment, the ysvH gene has at least about 95% identity with the sequence of SEQ ID NO:25. Accordingly, in one embodiment, the ysvH gene has at least about 85%, 86%, 87%, 88%, 89%, 90%, 91%, 92%, 93%, 94%, 95%, 96%, 97%, 98%, or 99% identity with the sequence of SEQ ID NO:25. In another embodiment, the ysvH gene comprises the sequence of SEQ ID NO:25. In yet another embodiment the ysvH gene consists of the sequence of SEQ ID NO:25.

In some embodiments, the transporter of lysine is encoded by a transporter of lysine gene derived from a bacterial genus or species, including but not limited to, Bacillus subtilis, Bacillus cereus, Bacillus stearothermophilus, Corynebacterium glutamicum, Escherichia coli, Lactococcus lactis, Pseudomonas aeruginosa, and Klebsiella pneumoniae. In some embodiments, the bacterial species is Escherichia coli. In some embodiments, the bacterial species is Escherichia coli strain Nissle.

Assays for testing the activity of a transporter of lysine, a functional variant of a transporter of lysine, or a functional fragment of transporter of lysine are well known to one of ordinary skill in the art. For example, import of lysine may be determined using the methods as described in Steffes et al. (1992) J. Bacteriol. 174: 3242-9, the entire contents of each of which are expressly incorporated by reference herein.

In one embodiment, when the transporter of a lysine is expressed in the recombinant bacterial cells described herein, the bacterial cells import 10% more lysine into the bacterial cell when the transporter is expressed than unmodified bacteria of the same bacterial subtype under the same conditions. In another embodiment, when the transporter of lysine is expressed in the recombinant bacterial cells described herein, the bacterial cells import 20%, 30%, 40%, 50%, 60%, 70%, 80%, 90% or 100% more lysine into the bacterial cell when the transporter is expressed than unmodified bacteria of the same bacterial subtype under the same conditions. In yet another embodiment, when the transporter of lysine is expressed in the recombinant bacterial cells described herein, the bacterial cells import two-fold more lysine into the cell when the transporter is expressed than unmodified bacteria of the same bacterial subtype under the same conditions. In yet another embodiment, when the transporter of lysine is expressed in the recombinant bacterial cells described herein, the bacterial cells import three-fold, four-fold, five-fold, six-fold, seven-fold, eight-fold, nine-fold, or ten-fold more lysine into the cell when the transporter is expressed than unmodified bacteria of the same bacterial subtype under the same conditions.

C. Exporters of Lysine

Lysine exporters may be modified in the recombinant bacteria described herein in order to reduce lysine export from the cell. Specifically, when the recombinant bacterial cells described herein comprise a genetic modification that reduces export of lysine, the bacterial cells retain more lysine in the bacterial cell than unmodified bacteria of the same bacterial subtype under the same conditions. Thus, the recombinant bacteria comprising a genetic modification that reduces export of lysine may be used to retain more lysine in the bacterial cell so that any lysine catabolism enzyme expressed in the organism, e.g., co-expressed lysine aminotransferase, can catabolize the lysine. For example, the lysine carrier LysE regulates the cytoplasmic concentration of lysine by mediating its export from bacterial cells. Members of the LysE superfamily have been identified in many bacterial species including, e.g., Escherichia coli, Corynebacterium glutamicum, Bacillus subtilis, Mycobacterium tuberculosis, Pseudomonas aeruginosa, and Helicobacter pylori (see, e.g., Vrljic et al. (1999) J. Mol. Microbiol. Biotechnol. 1(2): 327-36, the entire contents of which are incorporated herein by reference).

In one embodiment, the genetic modification is a mutation in an endogenous gene encoding an exporter of lysine. In one embodiment, the recombinant bacterial cell comprises a genetic modification that reduces export of lysine from the bacterial cell, wherein the endogenous gene encoding an exporter of lysine is a lysE gene. In one embodiment, the recombinant bacterial cell comprises a genetic modification that reduces export of lysine from the bacterial cell and a heterologous gene encoding an lysine catabolism enzyme. In one embodiment, the recombinant bacteria further comprise a heterologous gene encoding a transporter of lysine.

In one embodiment, the genetic modification reduces export of lysine from the bacterial cell. In one embodiment, the bacterial cell is from a bacterial genus or species that includes but is not limited to, Bacillus, Corynebacterium, Escherichia, Helicobacter, Mycobacterium, Pseudomonas, Escherichia coli, Corynebacterium glutamicum, Bacillus subtilis, Mycobacterium tuberculosis, Pseudomonas aeruginosa, and Helicobacter pylori. In another embodiment, the bacterial cell is an Escherichia coli bacterial cell. In another embodiment, the bacterial cell is an Escherichia coli strain Nissle bacterial cell.

In one embodiment, the lysE gene has at least about 80% identity with the sequence of SEQ ID NO:27. Accordingly, in one embodiment, the lysE gene has at least about 90% identity with the sequence of SEQ ID NO:27. Accordingly, in one embodiment, the lysE gene has at least about 95% identity with the sequence of SEQ ID NO:27. Accordingly, in one embodiment, the lysE gene has at least about 85%, 86%, 87%, 88%, 89%, 90%, 91%, 92%, 93%, 94%, 95%, 96%, 97%, 98%, or 99% identity with the sequence of SEQ ID NO:27. In another embodiment, the lysE gene comprises the sequence of SEQ ID NO:27. In yet another embodiment the lysE gene consists of the sequence of SEQ ID NO:27.

In one embodiment, the genetic mutation results in an exporter having reduced activity as compared to a wild-type exporter protein. In another embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export lysine from the bacterial cell. Assays for testing the activity of an exporter of a lysine are well known to one of ordinary skill in the art.

In another embodiment, the genetic modification is a mutation in a promoter of an endogenous gene encoding an exporter of lysine.

Assays for testing the activity of an exporter of lysine are well known to one of ordinary skill in the art. For example, export of arginine may be determined using the methods described by Vrljic et al. (1996) Mol. Microbiol. 22(5): 815-26, the entire contents of which are expressly incorporated herein by reference.

In yet another embodiment, the genetic modification is an overexpression of a repressor of an exporter of lysine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

4. Asparagine

A. Asparagine Catabolism Enzymes

Asparagine catabolism enzymes may be expressed or modified in the bacteria disclosed herein in order to enhance catabolism of asparagine. As used herein, the term “asparagine catabolism enzyme” refers to an enzyme involved in the catabolism of asparagine. Specifically, when an asparagine catabolism enzyme is expressed in a recombinant bacterial cell, the bacterial cell hydrolyzes more asparagine into aspartic acid when the catabolism enzyme is expressed than unmodified bacteria of the same bacterial subtype under the same conditions. Thus, the genetically engineered bacteria comprising a heterologous gene encoding an asparagine catabolism enzyme can catabolize asparagine to treat a disease associated with asparagine, such as cancer.

In one embodiment, the asparagine catabolism enzyme increases the rate of asparagine catabolism in the cell. In one embodiment, the asparagine catabolism enzyme decreases the level of asparagine in the cell. In another embodiment, the asparagine catabolism enzyme increases the level of aspartic acid in the cell.

Asparagine catabolism enzymes are well known to those of skill in the art (see, e.g., Spring et al. (1986) J. Bacteriol. 166: 135-42). In bacteria and plants, asparaginase enzymes (EC 3.5.1.1) are capable of converting asparagine to aspartic acid. For example, Escherichia coli contains two types of asparaginase: asparaginase I and asparaginase II. Asparaginase I is located in the cytoplasm, whereas asparaginase II is secreted. Asparaginase I has a relatively low affinity for asparagine, whereas asparagine II has a much higher affinity (see, e.g., Cedar and Schwartz (1967) J. Biol. Chem. 242: 3753-3755).

In some embodiments, an asparagine catabolism enzyme is encoded by a gene encoding an asparagine catabolism enzyme derived from a bacterial species. In some embodiments, an asparagine catabolism enzyme is encoded by a gene encoding an asparagine catabolism enzyme derived from a non-bacterial species. In some embodiments, an asparagine catabolism enzyme is encoded by a gene derived from a eukaryotic species, e.g., a yeast species or a plant species. In one embodiment, the gene encoding the asparagine catabolism enzyme is derived from an organism of the genus or species that includes, but is not limited to, Bacillus, Erwinia, Escherichia, Rhizobium, and Saccharomyces.

In one embodiment, the asparagine catabolism enzyme is an asparaginase. As used herein, “asparaginase” refers to any polypeptide having enzymatic activity that catalyzes the conversion of asparagine to aspartic acid and ammonia. For example, the asparaginase I enzyme of Escherichia coli (encoded by the ansA gene) is capable of metabolizing asparagine (see, e.g., Spring et al. (1986) J. Bacteriol. 166(1): 135-42; Jerlström et al. (1989) Gene 78(1): 37-46). Other distinct asparaginase enzymes are also known in the art (see, e.g., U.S. Pat. No. 7,396,670 B2, the entire contents of which are incorporated herein by reference).

In one embodiment, the asparaginase gene is derived from an organism of the genus or species that includes, but is not limited to Bacillus subtilis (Sun and Setlow (1991) J. Bacteriol. 173(12): 3831-45), Erwinia chrysanthemi, Escherichia coli, Rhizobium etli (Moreno-Enriquez (2012) J. Microbiol. Biotechnol 22(3): 292-300), and Saccharomyces (Jones (1977) J. Bacteriol. 130(1): 128-130).

In one embodiment, the asparagine catabolism enzyme is an asparaginase I enzyme. In one embodiment, the asparagine catabolism enzyme is an asparaginase II enzyme.

In one embodiment, the asparaginase gene is a ansA gene. In another embodiment, the ansA gene is a Escherichia coli ansA gene. In one embodiment, the asparaginase gene is a ansB gene. In another embodiment, the ansB gene is a Escherichia coli ansB gene.

In one embodiment, the ansA gene has at least about 80% identity with the sequence of SEQ ID NO:28. Accordingly, in one embodiment, the ansA gene has at least about 90% identity with the sequence of SEQ ID NO:28. Accordingly, in one embodiment, the ansA gene has at least about 95% identity with the sequence of SEQ ID NO:28. Accordingly, in one embodiment, the ansA gene has at least about 85%, 86%, 87%, 88%, 89%, 90%, 91%, 92%, 93%, 94%, 95%, 96%, 97%, 98%, or 99% identity with the sequence of SEQ ID NO:28. In another embodiment, the ansA gene comprises the sequence of SEQ ID NO:28. In yet another embodiment the ansA gene consists of the sequence of SEQ ID NO:28.

The present disclosure further comprises genes encoding functional fragments of an asparaginase gene or functional variants of an asparaginase gene.

Assays for testing the activity of an asparagine catabolism enzyme, an asparagine catabolism enzyme functional variant, or an asparagine catabolism enzyme functional fragment are well known to one of ordinary skill in the art. For example, asparagine catabolism can be assessed by expressing the protein, functional variant, or fragment thereof, in a recombinant bacterial cell that lacks endogenous asparagine catabolism enzyme activity. Asparagine catabolism can be assessed by measuring the conversion of _{L}-[U-¹⁴C] asparagine to _{L}-[U-¹⁴C] aspartate (see, e.g., Spring et al. (1986) J. Bacteriol. 166: 135-42), the entire contents of which are incorporated by reference).

In another embodiment, the gene encoding the asparaginase enzyme is co-expressed with an additional asparagine catabolism enzyme, for example, an asparaginase I enzyme is co-expressed with an asparaginase II enzyme.

In one embodiment, the bacterial cell comprises a heterologous gene encoding an asparagine catabolism enzyme. In one embodiment, the bacterial cell comprises a heterologous gene encoding a transporter of asparagine and a heterologous gene encoding an asparagine catabolism enzyme. In one embodiment, the bacterial cell comprises a heterologous gene encoding an asparagine catabolism enzyme and a genetic modification that reduces export of asparagine. In one embodiment, the bacterial cell comprises a heterologous gene encoding a transporter of asparagine, a heterologous gene encoding an asparagine catabolism enzyme, and a genetic modification that reduces export of asparagine. Transporters and exporters are described in more detail in the subsections, below.

B. Transporters of Asparagine

Asparagine transporters may be expressed or modified in the recombinant bacteria described herein in order to enhance asparagine transport into the cell. Specifically, when the transporter of asparagine is expressed in the recombinant bacterial cells described herein, the bacterial cells import more asparagine into the cell when the transporter is expressed than unmodified bacteria of the same bacterial subtype under the same conditions. Thus, the genetically engineered bacteria comprising a heterologous gene encoding transporter of asparagine which may be used to import asparagine into the bacteria so that any gene encoding an asparagine catabolism enzyme expressed in the organism, e.g., co-expressed asparaginase, can catabolize the asparagine to treat a disease, such as cancer.

The uptake of asparagine into bacterial cells is mediated by proteins well known to those of skill in the art. For example, two distinct systems for asparaginase uptake, distinguishable on the basis of their specificity for asparaginase have been identified in E. coli (see, e.g., Willis and Woolfolk (1975) J. Bacteriol. 123: 937-945). The bacterial gene ansP encodes an asparagine permease responsible for asparagine uptake in many bacteria (see, e.g., Jennings et al. (1995) Microbiology 141: 141-6; Ortuño-Olea and Durán-Vargas (2000) FEMS Microbiol. Lett. 189(2): 177-82; Barel et al. (2015) Front. Cell. Infect. Microbiol. 5: 9; and Gouzy et al. (2014) PLoS Pathog. 10(2): e1003928).

In one embodiment, the at least one gene encoding a transporter of asparagine is the ansP gene. In one embodiment, the bacterial cell described herein has been genetically engineered to comprise at least one heterologous ansP gene. In one embodiment, the at least one gene encoding a transporter of asparagine is the Escherichia coli ansP gene. In one embodiment, the at least one gene encoding a transporter of asparagine is the Francisella tularensis ansP gene. In one embodiment, the at least one gene encoding a transporter of asparagine is the Mycobacterium bovis ansP2 gene. In one embodiment, the at least one gene encoding a transporter of asparagine is the Salmonella enterica ansP gene. In one embodiment, the at least one gene encoding a transporter of asparagine is the Yersinia pestis ansP gene.

In one embodiment, the ansP2 gene has at least about 80% identity with the sequence of SEQ ID NO:29. Accordingly, in one embodiment, the ansP2 gene has at least about 90% identity with the sequence of SEQ ID NO:29. Accordingly, in one embodiment, the ansP2 gene has at least about 95% identity with the sequence of SEQ ID NO:29. Accordingly, in one embodiment, the ansP2 gene has at least about 85%, 86%, 87%, 88%, 89%, 90%, 91%, 92%, 93%, 94%, 95%, 96%, 97%, 98%, or 99% identity with the sequence of SEQ ID NO:29. In another embodiment, the ansP2 gene comprises the sequence of SEQ ID NO:29. In yet another embodiment the ansP2 gene consists of the sequence of SEQ ID NO:29.

In some embodiments, the transporter of asparagine is encoded by a transporter of asparagine gene derived from a bacterial genus or species, including but not limited to, Escherichia, Francisella, Mycobacterium, Salmonella, Yersinia, Escherichia coli, Francisella tularensis, Mycobacterium tuberculosis, Salmonella enterica, or Yersinia pestis. In some embodiments, the bacterial species is Escherichia coli. In some embodiments, the bacterial species is Escherichia coli strain Nissle.

Assays for testing the activity of a transporter of asparagine, a functional variant of a transporter of asparagine, or a functional fragment of transporter of asparagine are well known to one of ordinary skill in the art. For example, import of asparagine may be determined using the methods as described in Jennings et al. (1995) Microbiology 141: 141-6, the entire contents of each of which are expressly incorporated by reference herein.

In one embodiment, when the transporter of an asparagine is expressed in the recombinant bacterial cells described herein, the bacterial cells import 10% more asparagine into the bacterial cell when the transporter is expressed than unmodified bacteria of the same bacterial subtype under the same conditions. In another embodiment, when the transporter of asparagine is expressed in the recombinant bacterial cells described herein, the bacterial cells import 20%, 30%, 40%, 50%, 60%, 70%, 80%, 90% or 100% more asparagine into the bacterial cell when the transporter is expressed than unmodified bacteria of the same bacterial subtype under the same conditions. In yet another embodiment, when the transporter of asparagine is expressed in the recombinant bacterial cells described herein, the bacterial cells import two-fold more asparagine into the cell when the transporter is expressed than unmodified bacteria of the same bacterial subtype under the same conditions. In yet another embodiment, when the transporter of asparagine is expressed in the recombinant bacterial cells described herein, the bacterial cells import three-fold, four-fold, five-fold, six-fold, seven-fold, eight-fold, nine-fold, or ten-fold more asparagine into the cell when the transporter is expressed than unmodified bacteria of the same bacterial subtype under the same conditions.

C. Exporters of Asparagine

Asparagine exporters may be modified in the recombinant bacteria described herein in order to reduce asparagine export from the cell. Specifically, when the recombinant bacterial cells described herein comprise a genetic modification that reduces export of asparagine, the bacterial cells retain more asparagine in the bacterial cell than unmodified bacteria of the same bacterial subtype under the same conditions. Thus, the recombinant bacteria comprising a genetic modification that reduces export of asparagine may be used to retain more asparagine in the bacterial cell so that any asparagine catabolism enzyme expressed in the organism, e.g., co-expressed asparaginase, can catabolize the asparagine.

In one embodiment, the genetic modification is a mutation in an endogenous gene encoding an exporter of asparagine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export asparagine from the bacterial cell. Assays for testing the activity of an exporter of an asparagine are well known to one of ordinary skill in the art.

In another embodiment, the genetic modification is a mutation in a promoter of an endogenous gene encoding an exporter of asparagine.

In yet another embodiment, the genetic modification is an overexpression of a repressor of an exporter of asparagine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

5. Serine

A. Serine Catabolism Enzymes

Serine catabolism enzymes may be expressed or modified in the bacteria disclosed herein in order to enhance catabolism of serine. As used herein, the term “serine catabolism enzyme” refers to an enzyme involved in the catabolism of serine. Specifically, when a serine catabolism enzyme is expressed in a recombinant bacterial cell, the bacterial cell catabolizes more serine when the catabolism enzyme is expressed than unmodified bacteria of the same bacterial subtype under the same conditions. Thus, the genetically engineered bacteria comprising a heterologous gene encoding a serine catabolism enzyme can catabolize serine to treat a disease associated with serine, such as cancer.

In one embodiment, the serine catabolism enzyme increases the rate of serine catabolism in the cell. In one embodiment, the serine catabolism enzyme decreases the level of serine in the cell. In another embodiment, the serine catabolism enzyme increases the level of pyruvate in the cell. In one embodiment, the serine catabolism enzyme increases the level of glycine in the cell. In another embodiment, the serine catabolism enzyme increases the level of 5,10-methylenetetrahydrofolate in the cell.

Serine catabolism enzymes are well known to those of skill in the art (see, e.g., Florio et al. (2009) FEBS Journal 276: 132-43; Burman et al. (2004) FEBS Letters 576: 442-4; Netzer et al. (2004) Appl. Environ. Microbiol. 70(12): 7148-55; and Cicchillo et al. (2004) J. Biol. Chem. 279: 32418-25).

In some embodiments, a serine catabolism enzyme is encoded by a gene encoding a serine catabolism enzyme derived from a bacterial species. In some embodiments, a serine catabolism enzyme is encoded by a gene encoding a serine catabolism enzyme derived from a non-bacterial species. In some embodiments, a serine catabolism enzyme is encoded by a gene derived from a eukaryotic species, e.g., a yeast species or a plant species. In some embodiments, a serine catabolism enzyme is encoded by a gene derived from a plant species. In one embodiment, the gene encoding the serine catabolism enzyme is derived from an organism of the genus or species that includes, but is not limited to, Bacillus (e.g., Bacillus stearothermophilus and Bacillus subtilis), Escherichia (e.g., Escherichia coli), Klebsiella (e.g., Klebsiella pneumoniae), and Pseudomonas (e.g., Pseudomonas fluorescens).

In one embodiment, the serine catabolism enzyme is a serine deaminase (also known as L-serine ammonia lyase and serine dehydratase; E.C. 4.1.1.17). As used herein, “serine deaminase” refers to any polypeptide having enzymatic activity that catalyzes the deamination of serine to produce pyruvate and ammonia. Serine deaminases have been isolated and characterized from multiple organisms. Bacterial serine deaminases do not require pyroxisal phosphate, and instead have catalytically active [4Fe-4S]²⁺ clusters. Three serine deaminases have been identified in Escherichia coli, and are encoded by the sdaA (SdaA), sdaB (SdaB), and the multicistronic tdcABCDEFG operon (TdcG) (see, e.g., Burman et al. (2004) FEBS Letters 576: 442-4; Zhang et al. (2010) J. Bacteriol. 192: 5515-25; Shao and Newman (1993) Eur. J. Biochem. 212: 777-84; and Cicchillo et al. (2004)). Homologues of these enzymes have been identified and are known in the art.

In one embodiment, the at least one gene encoding a serine deaminase is a Bacillus subtilis serine deaminase gene. In one embodiment, the serine deaminase gene has at least about 80% identity with the sequence of SEQ ID NO:30. Accordingly, in one embodiment, the serine deaminase gene has at least about 90% identity with the sequence of SEQ ID NO:30. Accordingly, in one embodiment, the serine deaminase gene has at least about 95% identity with the sequence of SEQ ID NO:30. Accordingly, in one embodiment, the serine deaminase gene has at least about 85%, 86%, 87%, 88%, 89%, 90%, 91%, 92%, 93%, 94%, 95%, 96%, 97%, 98%, or 99% identity with the sequence of SEQ ID NO:30. In another embodiment, the serine deaminase gene comprises the sequence of SEQ ID NO:30. In yet another embodiment the serine deaminase gene consists of the sequence of SEQ ID NO:30.

In one embodiment, the at least one gene encoding a serine deaminase is the sdaA gene. In one embodiment, the bacterial cell described herein has been genetically engineered to comprise at least one heterologous sdaA gene. In one embodiment, the at least one gene encoding a serine deaminase is the Escherichia coli sdaA gene. In one embodiment, the at least one gene encoding a serine deaminase is the Pseudomonas fluorescens sdaA gene.

In one embodiment, the sdaA gene has at least about 80% identity with the sequence of SEQ ID NO:31. Accordingly, in one embodiment, the sdaA gene has at least about 90% identity with the sequence of SEQ ID NO:31. Accordingly, in one embodiment, the sdaA gene has at least about 95% identity with the sequence of SEQ ID NO:31. Accordingly, in one embodiment, the sdaA gene has at least about 85%, 86%, 87%, 88%, 89%, 90%, 91%, 92%, 93%, 94%, 95%, 96%, 97%, 98%, or 99% identity with the sequence of SEQ ID NO:31. In another embodiment, the sdaA gene comprises the sequence of SEQ ID NO:31. In yet another embodiment the sdaA gene consists of the sequence of SEQ ID NO:31.

In one embodiment, the at least one gene encoding a serine deaminase is the sdaB gene. In one embodiment, the bacterial cell described herein has been genetically engineered to comprise at least one heterologous sdaB gene. In one embodiment, the at least one gene encoding a serine deaminase is the Escherichia coli sdaB gene. In one embodiment, the at least one gene encoding a serine deaminase is the Klebsiella pneumoniae sdaB gene.

In one embodiment, the sdaB gene has at least about 80% identity with the sequence of SEQ ID NO:32. Accordingly, in one embodiment, the sdaB gene has at least about 90% identity with the sequence of SEQ ID NO:32. Accordingly, in one embodiment, the sdaB gene has at least about 95% identity with the sequence of SEQ ID NO:32. Accordingly, in one embodiment, the sdaB gene has at least about 85%, 86%, 87%, 88%, 89%, 90%, 91%, 92%, 93%, 94%, 95%, 96%, 97%, 98%, or 99% identity with the sequence of SEQ ID NO:32. In another embodiment, the sdaB gene comprises the sequence of SEQ ID NO:32. In yet another embodiment the sdaB gene consists of the sequence of SEQ ID NO:32.

In one embodiment, the at least one gene encoding a serine deaminase is the tdcG gene. In one embodiment, the bacterial cell described herein has been genetically engineered to comprise at least one heterologous tdcG gene. In one embodiment, the at least one gene encoding a serine deaminase is the Escherichia coli tdcG gene. In one embodiment, the at least one gene encoding a serine deaminase is the Escherichia coli tdcG gene.

In one embodiment, the tdcG gene has at least about 80% identity with the sequence of SEQ ID NO:33. Accordingly, in one embodiment, the tdcG gene has at least about 90% identity with the sequence of SEQ ID NO:33. Accordingly, in one embodiment, the tdcG gene has at least about 95% identity with the sequence of SEQ ID NO:33. Accordingly, in one embodiment, the tdcG gene has at least about 85%, 86%, 87%, 88%, 89%, 90%, 91%, 92%, 93%, 94%, 95%, 96%, 97%, 98%, or 99% identity with the sequence of SEQ ID NO:33. In another embodiment, the tdcG gene comprises the sequence of SEQ ID NO:33. In yet another embodiment the tdcG gene consists of the sequence of SEQ ID NO:33.

In one embodiment, the at least one gene encoding a serine deaminase is the tdcABCDEFG operon. In one embodiment, the bacterial cell described herein has been genetically engineered to comprise at least one heterologous tdcABCDEFG operon. In one embodiment, the at least one gene encoding a serine deaminase is the Escherichia coli tdcABCDEFG operon. In one embodiment, the at least one gene encoding a serine deaminase is the Escherichia coli tdcABCDEFG operon.

The present disclosure further comprises genes encoding functional fragments of a serine deaminase gene or functional variants of a serine deaminase gene.

The present disclosure also comprises genes encoding functional fragments of a serine deaminase gene or functional variants of an serine deaminase gene.

Assays for testing the activity of a serine deaminase enzyme, a serine deaminase enzyme functional variant, or a serine deaminase enzyme functional fragment are well known to one of ordinary skill in the art. For example, serine deaminase activity can be assessed by expressing the protein, functional variant, or fragment thereof, in a recombinant bacterial cell that lacks endogenous serine deaminase enzyme activity. Serine deaminase activity can then be assessed as described, e.g., in Burman et al. (2004) FEBS Letters 576: 442-4, the entire contents of which are incorporated by reference.

In one embodiment, the serine catabolism enzyme is a serine hydroxymethyltransferase. As used herein, “serine hydroxymethyltransferase” refers to any polypeptide having enzymatic activity that catalyzes the conversion of serine to produce glycine. In some embodiments, the serine hydroxymethyltransferase also catalyzes the conversion of tetrahydrofolate to 5,10-methylenetetrahydrofolate. Serine hydroxymethyltransferases have been isolated and characterized from multiple organisms and are known in the art (see, e.g., Florio et al. (2009) FEBS Journal 276: 132-43).

In one embodiment, the at least one gene encoding a serine hydroxymethyltransferase is the glyA gene. In one embodiment, the bacterial cell described herein has been genetically engineered to comprise at least one heterologous glyA gene. In one embodiment, the at least one gene encoding a serine hydroxymethyltransferase is a Escherichia coli glyA gene. In one embodiment, the at least one gene encoding a serine hydroxymethyltransferase is a Bacillus stearothermophilus glyA gene.

In one embodiment, the glyA gene has at least about 80% identity with the sequence of SEQ ID NO:34. Accordingly, in one embodiment, the glyA gene has at least about 90% identity with the sequence of SEQ ID NO:34. Accordingly, in one embodiment, the glyA gene has at least about 95% identity with the sequence of SEQ ID NO:34. Accordingly, in one embodiment, the glyA gene has at least about 85%, 86%, 87%, 88%, 89%, 90%, 91%, 92%, 93%, 94%, 95%, 96%, 97%, 98%, or 99% identity with the sequence of SEQ ID NO:34. In another embodiment, the glyA gene comprises the sequence of SEQ ID NO:34. In yet another embodiment the glyA gene consists of the sequence of SEQ ID NO:34.

The present disclosure further comprises genes encoding functional fragments of a serine hydroxymethyltransferase gene or functional variants of a serine hydroxymethyltransferase gene.

The present disclosure also comprises genes encoding functional fragments of a serine deaminase gene or functional variants of an serine hydroxymethyltransferase gene.

Assays for testing the activity of a serine hydroxymethyltransferase enzyme, a serine hydroxymethyltransferase enzyme functional variant, or a serine hydroxymethyltransferase enzyme functional fragment are well known to one of ordinary skill in the art. For example, serine hydroxymethyltransferase activity can be assessed by expressing the protein, functional variant, or fragment thereof, in a recombinant bacterial cell that lacks endogenous serine hydroxymethyltransferase enzyme activity. Serine hydroxymethyltransferase activity can then be assessed as described, e.g., in Florio et al. (2009) FEBS Journal 276: 132-43, the entire contents of which are incorporated by reference.

B. Transporters of Serine

Serine transporters may be expressed or modified in the recombinant bacteria described herein in order to enhance serine transport into the cell. Specifically, when the transporter of serine is expressed in the recombinant bacterial cells described herein, the bacterial cells import more serine into the cell when thetransporter is expressed than unmodified bacteria of the same bacterial subtype under the same conditions. Thus, the genetically engineered bacteria comprising a heterologous gene encoding transporter of serine which may be used to import serine into the bacteria so that any gene encoding a serine catabolism enzyme expressed in the organism, e.g., co-expressed serine deaminase, can catabolize the serine to treat a disease, such as cancer.

The uptake of serine into bacterial cells is mediated by proteins well known to those of skill in the art. For example, SdaC (encoded by the sdaC gene; also known as DcrA) is an inner membrane threonine-insensitive serine transporter that was originally identified in Escherichia coli (Shao et al. (1994) Eur. J. Biochem. 222: 901-7). Additional serine transporters that have been identified include the Na⁺/serine symporter, SstT (encoded by the sstT gene), the leucine-isoleucine-valine transporter LIV-1, which transports serine slowly, and the H⁺/serine-threonine symporter TdcC (encoded by the tdcC gene) (see, e.g., Ogawa et al. (1998) J. Bacteriol. 180: 6749-52; Ogawa et al. (1997) J. Biochem. 122(6): 1241-5).

In one embodiment, the at least one gene encoding a transporter of serine is the sdaC gene. In one embodiment, the bacterial cell described herein has been genetically engineered to comprise at least one heterologous sdaC gene. In one embodiment, the at least one gene encoding a transporter of serine is the Escherichia coli sdaC gene. In one embodiment, the at least one gene encoding a transporter of serine is the Campylobacter jejuni sdaC gene.

In one embodiment, the sdaC gene has at least about 80% identity with the sequence of SEQ ID NO:35. Accordingly, in one embodiment, the sdaC gene has at least about 90% identity with the sequence of SEQ ID NO:35. Accordingly, in one embodiment, the sdaC gene has at least about 95% identity with the sequence of SEQ ID NO:35. Accordingly, in one embodiment, the sdaC gene has at least about 85%, 86%, 87%, 88%, 89%, 90%, 91%, 92%, 93%, 94%, 95%, 96%, 97%, 98%, or 99% identity with the sequence of SEQ ID NO:35. In another embodiment, the sdaC gene comprises the sequence of SEQ ID NO:35. In yet another embodiment the sdaC gene consists of the sequence of SEQ ID NO:35.

In one embodiment, the at least one gene encoding a transporter of serine is the sstT gene. In one embodiment, the bacterial cell described herein has been genetically engineered to comprise at least one heterologous sstT gene. In one embodiment, the at least one gene encoding a transporter of serine is the Escherichia coli sstT gene.

In one embodiment, the at least one gene encoding a transporter of serine is the tdcC gene. In one embodiment, the bacterial cell described herein has been genetically engineered to comprise at least one heterologous tdcC gene. In one embodiment, the at least one gene encoding a transporter of serine is the Escherichia coli tdcC gene.

In some embodiments, the transporter of serine is encoded by a transporter of serine gene derived from a bacterial genus or species, including but not limited to, Campylobacter, Campylobacter jejuni, Escherichia, and Escherichia coli In some embodiments, the bacterial species is Escherichia coli. In some embodiments, the bacterial species is Escherichia coli strain Nissle.

Assays for testing the activity of a transporter of serine, a functional variant of a transporter of serine, or a functional fragment of transporter of serine are well known to one of ordinary skill in the art. For example, import of serine may be determined using the methods as described in Hama et al. (1987) Biochim. Biophys. Acta 905: 231-9, the entire contents of each of which are expressly incorporated by reference herein.

In one embodiment, when the transporter of a serine is expressed in the recombinant bacterial cells described herein, the bacterial cells import 10% more serine into the bacterial cell when the transporter is expressed than unmodified bacteria of the same bacterial subtype under the same conditions. In another embodiment, when the transporter of serine is expressed in the recombinant bacterial cells described herein, the bacterial cells import 20%, 30%, 40%, 50%, 60%, 70%, 80%, 90% or 100% more serine into the bacterial cell when the transporter is expressed than unmodified bacteria of the same bacterial subtype under the same conditions. In yet another embodiment, when the transporter of serine is expressed in the recombinant bacterial cells described herein, the bacterial cells import two-fold more serine into the cell when the transporter is expressed than unmodified bacteria of the same bacterial subtype under the same conditions. In yet another embodiment, when the transporter of serine is expressed in the recombinant bacterial cells described herein, the bacterial cells import three-fold, four-fold, five-fold, six-fold, seven-fold, eight-fold, nine-fold, or ten-fold more serine into the cell when the transporter is expressed than unmodified bacteria of the same bacterial subtype under the same conditions.

C. Exporters of Serine

Serine exporters may be modified in the recombinant bacteria described herein in order to reduce serine export from the cell. Specifically, when the recombinant bacterial cells described herein comprise a genetic modification that reduces export of serine, the bacterial cells retain more serine in the bacterial cell than unmodified bacteria of the same bacterial subtype under the same conditions. Thus, the recombinant bacteria comprising a genetic modification that reduces export of serine may be used to retain more serine in the bacterial cell so that any serine catabolism enzyme expressed in the organism, e.g., co-expressed serine deaminase, can catabolize the serine. For example, the serine/threonine exporter ThrE (encoded by the thrE gene) mediates the export of serine from bacterial cells (Simic et al. (2001) J. Bacteriol. 183: 5317-24; Simic et al. (2002) Appl. Environ. Microbiol. 68(7): 3321-3327). ThrE homologues have been identified in multiple bacterial species, including Corynebacterium glutamicum, Mycobacterium tuberculosis, and Streptomyces coelicolor.

In one embodiment, the genetic modification is a mutation in an endogenous gene encoding an exporter of serine. In one embodiment, the recombinant bacterial cell of the invention comprises a genetic modification that reduces export of serine from the bacterial cell, wherein the endogenous gene encoding an exporter of serine is a ThrE gene. In one embodiment, the recombinant bacterial cell of the invention comprises a genetic modification that reduces export of serine from the bacterial cell, wherein the endogenous gene encoding an exporter of serine is at least 80% homologous to the gene of SEQ ID NO: 36. In one embodiment, the recombinant bacterial cell of the invention comprises a genetic modification that reduces export of serine from the bacterial cell and a heterologous gene encoding an serine catabolism enzyme. In one embodiment, the recombinant bacteria further comprise a heterologous gene encoding a transporter of serine.

In one embodiment, the genetic modification reduces export of serine from the bacterial cell. In one embodiment, the bacterial cell is from a bacterial genus or species that includes but is not limited to, Corynebacterium, Corynebacterium glutamicum, Escherichia, Lactobacillus, Lactobacillus saniviri, Mycobacterium, Mycobacterium tuberculosis, and Streptomyces, Streptomyces coelicolor. In another embodiment, the bacterial cell is an Escherichia coli bacterial cell. In another embodiment, the bacterial cell is an Escherichia coli strain Nissle bacterial cell.

In one embodiment, the genetic mutation results in an exporter having reduced activity as compared to a wild-type exporter protein. In another embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export serine from the bacterial cell. Assays for testing the activity of an exporter of a serine are well known to one of ordinary skill in the art.

In another embodiment, the genetic modification is a mutation in a promoter of an endogenous gene encoding an exporter of serine.

Assays for testing the activity of an exporter of serine are well known to one of ordinary skill in the art. For example, export of serine may be determined using the methods described by Simic et al. (2001) J. Bacteriol. 183: 5317-24, the entire contents of which are hereby incorporated by reference, the entire contents of which are expressly incorporated herein by reference.

In yet another embodiment, the genetic modification is an overexpression of a repressor of an exporter of serine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

6. Glutamine

A. Glutamine Catabolism Enzymes

Glutamine catabolism enzymes may be expressed or modified in the bacteria disclosed herein in order to enhance catabolism of glutamine. As used herein, the term “glutamine catabolism enzyme” refers to an enzyme involved in the catabolism of glutamine Specifically, when a glutamine catabolism enzyme is expressed in a recombinant bacterial cell, the bacterial cell hydrolyzes more glutamine into glutamate when the catabolism enzyme is expressed than unmodified bacteria of the same bacterial subtype under the same conditions. Thus, the genetically engineered bacteria comprising a heterologous gene encoding a glutamine catabolism enzyme can catabolize glutamine to treat a disease associated with glutamine, such as cancer. Indeed, human fibroblasts with activated c-MYC have been shown to depend on glutamine.

In one embodiment, the glutamine catabolism enzyme increases the rate of glutamine catabolism in the cell. In one embodiment, the glutamine catabolism enzyme decreases the level of glutamine in the cell. In another embodiment, the glutamine catabolism enzyme increases the level of glutamate in the cell.

Glutamine catabolism enzymes are well known to those of skill in the art (see, e.g., Brown et al., Biochemistry, 47(21):5724-5735, 2008). For example, the YbaS and YneH glutaminases have been identified in Escherichia coli, and the YlaM and YbgJ glutaminases have been identified in Bacillus subtilis.

In some embodiments, a glutamine catabolism enzyme is encoded by a gene encoding a glutamine catabolism enzyme derived from a bacterial species. In some embodiments, a glutamine catabolism enzyme is encoded by a gene encoding a glutamine catabolism enzyme derived from a non-bacterial species. In some embodiments, a glutamine catabolism enzyme is encoded by a gene derived from a eukaryotic species, e.g., a yeast species or a plant species. In one embodiment, the gene encoding the glutamine catabolism enzyme is derived from an organism of the genus or species that includes, but is not limited to, Bacillus subtilis and Escherichia coli.

In one embodiment, the glutamine catabolism enzyme is a glutaminase. As used herein, the term “glutaminase” refers to an enzyme capable of hydrolytic deamidation of L-glutamine to L-glutamate (see, e.g., Brown et al., Biochemistry, 47(21):5724-5735, 2008).

In one embodiment, the glutaminase gene is a ybaS gene. In another embodiment, the glutaminase gene is a ybaS gene from Escherichia coli. In one embodiment, the glutaminase gene is a yneH gene. In another embodiment, the glutaminase gene is a yneH gene from Escherichia coli. In one embodiment, the glutaminase gene is a ylaM gene. In another embodiment, the glutaminase gene is a ylaM gene from Bacillus subtilis. In one embodiment, the glutaminase gene is a ybgJ gene. In another embodiment, the glutaminase gene is a ybgJ gene from Bacillus subtilis.

In one embodiment, the glutamine transaminase gene has at least about 80% identity with the sequence of any one of SEQ ID NOs:37-40. Accordingly, in one embodiment, the glutamine transaminase gene has at least about 90% identity with the sequence of any one of SEQ ID NOs:37-40. Accordingly, in one embodiment, the glutamine transaminase gene has at least about 95% identity with the sequence of any one of SEQ ID NOs:37-40. Accordingly, in one embodiment, the glutamine transaminase gene has at least about 85%, 86%, 87%, 88%, 89%, 90%, 91%, 92%, 93%, 94%, 95%, 96%, 97%, 98%, or 99% identity with the sequence of any one of SEQ ID NOs:37-40. In another embodiment, the glutamine transaminase gene comprises the sequence of any one of SEQ ID NOs:37-40. In yet another embodiment the glutamine transaminase gene consists of the sequence of any one of SEQ ID NOs:37-40.

The present disclosure further comprises genes encoding functional fragments of a glutamine amino acid catabolism enzyme or functional variants of a glutamine amino acid catabolism enzyme.

Assays for testing the activity of a glutamine catabolism enzyme, a glutamine catabolism enzyme functional variant, or a glutamine catabolism enzyme functional fragment are well known to one of ordinary skill in the art. For example, glutamine catabolism can be assessed by expressing the protein, functional variant, or fragment thereof, in a recombinant bacterial cell that lacks endogenous glutamine catabolism enzyme activity. Other methods are also well known to one of ordinary skill in the art (see, e.g., Brown et al., Biochemistry, 47(21):5724-5735, 2008, the entire contents of which are incorporated by reference).

In one embodiment, the bacterial cell comprises a heterologous gene encoding a glutamine catabolism enzyme. In one embodiment, the bacterial cell comprises a heterologous gene encoding a transporter of glutamine and a heterologous gene encoding a glutamine catabolism enzyme. In one embodiment, the bacterial cell comprises a heterologous gene encoding a glutamine catabolism enzyme and a genetic modification that reduces export of glutamine. In one embodiment, the bacterial cell comprises a heterologous gene encoding a transporter of glutamine, a heterologous gene encoding a glutamine catabolism enzyme, and a genetic modification that reduces export of glutamine Transporters and exporters are described in more detail in the subsections, below.

B. Transporters of Glutamine

Glutamine transporters may be expressed or modified in the recombinant bacteria described herein in order to enhance glutamine transport into the cell. Specifically, when the transporter of glutamine is expressed in the recombinant bacterial cells described herein, the bacterial cells import more glutamine into the cell when the transporter is expressed than unmodified bacteria of the same bacterial subtype under the same conditions. Thus, the genetically engineered bacteria comprising a heterologous gene encoding transporter of glutamine which may be used to import glutamine into the bacteria so that any gene encoding a glutamine catabolism enzyme expressed in the organism can catabolize the glutamine to treat a disease associated with glutamine, such as cancer.

The uptake of glutamine into bacterial cells is mediated by proteins well known to those of skill in the art. For example, a glutamine permease glnHPQ operon has been identified in Escherichia coli (Nohno et al., Mol. Gen. Genet., 205(2):260-269, 1986).

In one embodiment, the at least one gene encoding a transporter of glutamine is the glnHPQ operon. In one embodiment, the bacterial cell described herein has been genetically engineered to comprise at least one heterologous gene from the glnHPQ operon. In one embodiment, the bacterial cell described herein has been genetically engineered to comprise at least one heterologous glnH gene. In one embodiment, the bacterial cell described herein has been genetically engineered to comprise at least one heterologous glnP gene. In one embodiment, the bacterial cell described herein has been genetically engineered to comprise at least one heterologous glnQ gene.

In one embodiment, the glnHPQ operon has at least about 80% identity with the sequence of SEQ ID NO:41. Accordingly, in one embodiment, the glnHPQ operon has at least about 90% identity with the sequence of SEQ ID NO:41. Accordingly, in one embodiment, the glnHPQ operon has at least about 95% identity with the sequence of SEQ ID NO:41. Accordingly, in one embodiment, the glnHPQ operon has at least about 85%, 86%, 87%, 88%, 89%, 90%, 91%, 92%, 93%, 94%, 95%, 96%, 97%, 98%, or 99% identity with the sequence of SEQ ID NO:41. In another embodiment, the glnHPQ operon comprises the sequence of SEQ ID NO:41. In yet another embodiment the glnHPQ operon consists of the sequence of SEQ ID NO:41.

In one embodiment, the glnH gene has at least about 80% identity with the sequence of SEQ ID NO:42. Accordingly, in one embodiment, the glnH gene has at least about 90% identity with the sequence of SEQ ID NO:42. Accordingly, in one embodiment, the glnH gene has at least about 95% identity with the sequence of SEQ ID NO:42. Accordingly, in one embodiment, the glnH gene has at least about 85%, 86%, 87%, 88%, 89%, 90%, 91%, 92%, 93%, 94%, 95%, 96%, 97%, 98%, or 99% identity with the sequence of SEQ ID NO:42. In another embodiment, the glnH gene comprises the sequence of SEQ ID NO:42. In yet another embodiment the glnH gene consists of the sequence of SEQ ID NO:42.

In one embodiment, the glnP gene has at least about 80% identity with the sequence of SEQ ID NO:43. Accordingly, in one embodiment, the glnP gene has at least about 90% identity with the sequence of SEQ ID NO:43. Accordingly, in one embodiment, the glnP gene has at least about 95% identity with the sequence of SEQ ID NO:43. Accordingly, in one embodiment, the glnP gene has at least about 85%, 86%, 87%, 88%, 89%, 90%, 91%, 92%, 93%, 94%, 95%, 96%, 97%, 98%, or 99% identity with the sequence of SEQ ID NO:43. In another embodiment, the glnP gene comprises the sequence of SEQ ID NO:43. In yet another embodiment the glnP gene consists of the sequence of SEQ ID NO:43.

In one embodiment, the glnQ gene has at least about 80% identity with the sequence of SEQ ID NO:44. Accordingly, in one embodiment, the glnQ gene has at least about 90% identity with the sequence of SEQ ID NO:44. Accordingly, in one embodiment, the glnQ gene has at least about 95% identity with the sequence of SEQ ID NO:44. Accordingly, in one embodiment, the glnQ gene has at least about 85%, 86%, 87%, 88%, 89%, 90%, 91%, 92%, 93%, 94%, 95%, 96%, 97%, 98%, or 99% identity with the sequence of SEQ ID NO:44. In another embodiment, the glnQ gene comprises the sequence of SEQ ID NO:44. In yet another embodiment the glnQ gene consists of the sequence of SEQ ID NO:44.

In some embodiments, the transporter of glutamine is encoded by a transporter of glutamine gene derived from a bacterial genus or species, including but not limited to, Escherichia coli. In some embodiments, the bacterial species is Escherichia coli strain Nissle.

Assays for testing the activity of a transporter of glutamine, a functional variant of a transporter of glutamine, or a functional fragment of transporter of glutamine are well known to one of ordinary skill in the art. For example, import of glutamine may be determined using the methods as described in Nohno et al., Mol. Gen. Genet., 205(2):260-269, 1986, the entire contents of which are expressly incorporated by reference herein.

In one embodiment, when the transporter of a glutamine is expressed in the recombinant bacterial cells described herein, the bacterial cells import 10% more glutamine into the bacterial cell when the transporter is expressed than unmodified bacteria of the same bacterial subtype under the same conditions. In another embodiment, when the transporter of glutamine is expressed in the recombinant bacterial cells described herein, the bacterial cells import 20%, 30%, 40%, 50%, 60%, 70%, 80%, 90% or 100% more glutamine into the bacterial cell when the transporter is expressed than unmodified bacteria of the same bacterial subtype under the same conditions. In yet another embodiment, when the transporter of glutamine is expressed in the recombinant bacterial cells described herein, the bacterial cells import two-fold more glutamine into the cell when the transporter is expressed than unmodified bacteria of the same bacterial subtype under the same conditions. In yet another embodiment, when the transporter of glutamine is expressed in the recombinant bacterial cells described herein, the bacterial cells import three-fold, four-fold, five-fold, six-fold, seven-fold, eight-fold, nine-fold, or ten-fold more glutamine into the cell when the transporter is expressed than unmodified bacteria of the same bacterial subtype under the same conditions.

C. Exporters of Glutamine

Glutamine exporters may be modified in the recombinant bacteria described herein in order to reduce glutamine export from the cell. Specifically, when the recombinant bacterial cells described herein comprise a genetic modification that reduces export of glutamine, the bacterial cells retain more glutamine in the bacterial cell than unmodified bacteria of the same bacterial subtype under the same conditions. Thus, the recombinant bacteria comprising a genetic modification that reduces export of glutamine may be used to retain more glutamine in the bacterial cell so that any glutamine catabolism enzyme expressed in the organism, e.g., co-expressed glutamine catabolism enzyme, can catabolize the glutamine.

In one embodiment, the genetic modification is a mutation in an endogenous gene encoding an exporter of glutamine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export glutamine from the bacterial cell.

In another embodiment, the genetic modification is a mutation in a promoter of an endogenous gene encoding an exporter of glutamine

In yet another embodiment, the genetic modification is an overexpression of a repressor of an exporter of glutamine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

7. Tryptophan

A. Tryptophan Catabolism Enzymes

Tryptophan catabolism enzymes may be expressed or modified in the bacteria disclosed herein in order to enhance catabolism of tryptophan. As used herein, the term “tryptophan catabolism enzyme” refers to an enzyme involved in the catabolism of tryptophan. Specifically, when a tryptophan catabolism enzyme is expressed in a recombinant bacterial cell, the bacterial cell catabolizes more tryptophan when the catabolism enzyme is expressed than unmodified bacteria of the same bacterial subtype under the same conditions. Thus, the genetically engineered bacteria comprising at least one heterologous gene encoding a tryptophan catabolism enzyme can catabolize tryptophan to treat a disease associated with tryptophan, such as cancer, e.g., lymphoblastic leukemia.

In one embodiment, the tryptophan catabolism enzyme increases the rate of tryptophan catabolism in the cell. In one embodiment, the tryptophan catabolism enzyme decreases the level of tryptophan in the cell.

Tryptophan catabolism enzymes are well known to those of skill in the art (see, e.g., Aklujkar et al., Microbiology, 160:2694-2709, 2014). For example, a tryptophan transaminase enzyme has been identified in Ferroglobus placidus. Additionally, a tryptophan amino transferase (transaminase) has been identified in Ustilago maydis.

In some embodiments, a tryptophan catabolism enzyme is encoded by a gene encoding a tryptophan catabolism enzyme derived from a bacterial species. In some embodiments, a tryptophan catabolism enzyme is encoded by a gene encoding a tryptophan catabolism enzyme derived from a non-bacterial species. In some embodiments, a tryptophan catabolism enzyme is encoded by a gene derived from a eukaryotic species, e.g., a yeast species or a plant species. In one embodiment, the gene encoding the tryptophan catabolism enzyme is derived from an organism of the genus or species that includes, but is not limited to, Ferroglobus placidus and Ustilago maydis.

In one embodiment, the at least one tryptophan catabolism enzyme comprises an tryptophan amino transferase (transaminase). In one embodiment, the tryptophan amino transferase (transaminase) gene is from Ustilago maydis.

In one embodiment, the tryptophan amino transferase (transaminase) gene has at least about 80% identity with the sequence of SEQ ID NO:45. Accordingly, in one embodiment, the tryptophan amino transferase (transaminase) gene has at least about 90% identity with the sequence of SEQ ID NO:45. Accordingly, in one embodiment, the tryptophan amino transferase (transaminase) gene has at least about 95% identity with the sequence of SEQ ID NO:45. Accordingly, in one embodiment, the tryptophan amino transferase (transaminase) gene has at least about 85%, 86%, 87%, 88%, 89%, 90%, 91%, 92%, 93%, 94%, 95%, 96%, 97%, 98%, or 99% identity with the sequence of SEQ ID NO:45. In another embodiment, the tryptophan amino transferase (transaminase) gene comprises the sequence of SEQ ID NO:45. In yet another embodiment the tryptophan amino transferase (transaminase) gene consists of the sequence of SEQ ID NO:45.

The present disclosure further comprises genes encoding functional fragments of a tryptophan amino acid catabolism enzyme or functional variants of a tryptophan amino acid catabolism enzyme.

Assays for testing the activity of a tryptophan catabolism enzyme, a tryptophan catabolism enzyme functional variant, or a tryptophan catabolism enzyme functional fragment are well known to one of ordinary skill in the art. For example, tryptophan catabolism can be assessed by expressing the protein, functional variant, or fragment thereof, in a recombinant bacterial cell that lacks endogenous tryptophan catabolism enzyme activity. Other methods are also well known to one of ordinary skill in the art (see, e.g., Aklujkar et al., Microbiology, 160:2694-2709, 2014, the entire contents of which are incorporated by reference).

In one embodiment, the bacterial cell comprises a heterologous gene encoding a tryptophan catabolism enzyme. In one embodiment, the bacterial cell comprises a heterologous gene encoding a transporter of tryptophan and a heterologous gene encoding a tryptophan catabolism enzyme. In one embodiment, the bacterial cell comprises a heterologous gene encoding a tryptophan catabolism enzyme and a genetic modification that reduces export of tryptophan. In one embodiment, the bacterial cell comprises a heterologous gene encoding a transporter of tryptophan, a heterologous gene encoding a tryptophan catabolism enzyme, and a genetic modification that reduces export of tryptophan. Transporters and exporters are described in more detail in the subsections, below.

B. Transporters of Tryptophan

Tryptopha transporters may be expressed or modified in the recombinant bacteria described herein in order to enhance tryptophan transport into the cell. Specifically, when the transporter of tryptophan is expressed in the recombinant bacterial cells described herein, the bacterial cells import more tryptophan into the cell when the transporter is expressed than unmodified bacteria of the same bacterial subtype under the same conditions. Thus, the genetically engineered bacteria comprising a heterologous gene encoding transporter of tryptophan which may be used to import tryptophan into the bacteria so that any gene encoding an tryptophan catabolism enzyme expressed in the organism, e.g., co-expressed tryptophan amino transferase, can catabolize the tryptophan to treat a disease, such as cancer.

The uptake of tryptophan into bacterial cells is mediated by proteins well known to those of skill in the art. For example, three different transporters for tryptophan uptake, distinguishable on the basis of their affinity for tryptophan have been identified in E. coli (see, e.g., Yanofsky et al. (1991) J. Bacteriol. 173: 6009-17). The bacterial genes mtr, aroP, and tnaB encodes tryptophan permeases responsible for tryptophan uptake in bacteria. High affinity permease, Mtr, is negatively regulated by the trp repressor and positively regulated by the TyR product (see, e.g., Yanofsky et al. (1991) J. Bacteriol. 173: 6009-17 and Heatwole et al. (1991) J. Bacteriol. 173: 3601-04), while AroP is negatively regulated by the tyR product (Chye et al. (1987) J. Bacteriol. 169:386-93).

In one embodiment, the at least one gene encoding a transporter of tryptophan is selected from the mtr, aroP or tnaB genes. In one embodiment, the bacterial cell described herein has been genetically engineered to comprise at least one heterologous selected from the mtr, aroP or tnaB genes. In one embodiment, the at least one gene encoding a transporter of tryptophan is the Escherichia coli mtr, aroP or tnaB genes.

In one embodiment, the mtr gene has at least about 80% identity with the sequence of SEQ ID NO:46. Accordingly, in one embodiment, the mtr gene has at least about 90% identity with the sequence of SEQ ID NO:46. Accordingly, in one embodiment, the mtr gene has at least about 95% identity with the sequence of SEQ ID NO:46. Accordingly, in one embodiment, the mtr gene has at least about 85%, 86%, 87%, 88%, 89%, 90%, 91%, 92%, 93%, 94%, 95%, 96%, 97%, 98%, or 99% identity with the sequence of SEQ ID NO:46. In another embodiment, the mtr gene comprises the sequence of SEQ ID NO:46. In yet another embodiment the mtr gene consists of the sequence of SEQ ID NO:46.

In one embodiment, the tnaB gene has at least about 80% identity with the sequence of SEQ ID NO:47. Accordingly, in one embodiment, the tnaB gene has at least about 90% identity with the sequence of SEQ ID NO:47. Accordingly, in one embodiment, the tnaB gene has at least about 95% identity with the sequence of SEQ ID NO:47. Accordingly, in one embodiment, the tnaB gene has at least about 85%, 86%, 87%, 88%, 89%, 90%, 91%, 92%, 93%, 94%, 95%, 96%, 97%, 98%, or 99% identity with the sequence of SEQ ID NO:47. In another embodiment, the tnaB gene comprises the sequence of SEQ ID NO:47. In yet another embodiment the tnaB gene consists of the sequence of SEQ ID NO:47.

In one embodiment, the aroP gene has at least about 80% identity with the sequence of SEQ ID NO:48. Accordingly, in one embodiment, the aroP gene has at least about 90% identity with the sequence of SEQ ID NO:48. Accordingly, in one embodiment, the aroP gene has at least about 95% identity with the sequence of SEQ ID NO:48. Accordingly, in one embodiment, the aroP gene has at least about 85%, 86%, 87%, 88%, 89%, 90%, 91%, 92%, 93%, 94%, 95%, 96%, 97%, 98%, or 99% identity with the sequence of SEQ ID NO:48. In another embodiment, the aroP gene comprises the sequence of SEQ ID NO:48. In yet another embodiment the aroP gene consists of the sequence of SEQ ID NO:48.

In some embodiments, the transporter of tryptophan is encoded by a transporter of tryptophan gene derived from a bacterial genus or species, including but not limited to, Escherichia, Corynebacterium, Escherichia coli, Saccharomyces cerevisiae or Corynebacterium glutamicum. In some embodiments, the bacterial species is Escherichia coli. In some embodiments, the bacterial species is Escherichia coli strain Nissle.

Assays for testing the activity of a transporter of tryptophan, a functional variant of a transporter of tryptophan, or a functional fragment of transporter of tryptophan are well known to one of ordinary skill in the art. For example, import of tryptophan may be determined using the methods as described in Shang et al. (2013) J. Bacteriol. 195:5334-42, the entire contents of each of which are expressly incorporated by reference herein.

In one embodiment, when the transporter of a tryptophan is expressed in the recombinant bacterial cells described herein, the bacterial cells import 10% more tryptophan into the bacterial cell when the transporter is expressed than unmodified bacteria of the same bacterial subtype under the same conditions. In another embodiment, when the transporter of tryptophan is expressed in the recombinant bacterial cells described herein, the bacterial cells import 20%, 30%, 40%, 50%, 60%, 70%, 80%, 90% or 100% more tryptophan into the bacterial cell when the transporter is expressed than unmodified bacteria of the same bacterial subtype under the same conditions. In yet another embodiment, when the transporter of tryptophan is expressed in the recombinant bacterial cells described herein, the bacterial cells import two-fold more tryptophan into the cell when the transporter is expressed than unmodified bacteria of the same bacterial subtype under the same conditions. In yet another embodiment, when the transporter of tryptophan is expressed in the recombinant bacterial cells described herein, the bacterial cells import three-fold, four-fold, five-fold, six-fold, seven-fold, eight-fold, nine-fold, or ten-fold more tryptophan into the cell when the transporter is expressed than unmodified bacteria of the same bacterial subtype under the same conditions.

C. Exporters of Tryptophan

The export of tryptophan from bacterial cells is mediated by proteins well known to those of skill in the art. Salmonella enterica ser. Typhimurium and Escherichia coli were shown to have the ability to export tryptophan (see, e.g., Doroshenko et al. (2007) FEMS Microbiol. Lett. 275:312-18 and Nikaido (2003) Microbiol. Mol. Biol. Rev. 67:593-656). YddG is an aromatic amino acid exporter and is a member of the Paraquat (Methyl viologen) Exporter (PE) Family (TC: 2.A.7.17) within the Drug/Metabolite Transporter (DMT) superfamily. YddG of Salmonella typhimurium have 95% identity with E. coli YddG. In Salmonella typhimurium, YddG works with OmpD, which is a porin protein, to excrete methyl viologen (Santiviago et al. (2002) Mol. Microbiol. 46:687-98). OmpD porin forms a multiprotein complex with YddG to form an exit channel Expression of yddG from a multicopy plasmid resulted in increased resistance to phenylalanine, DL-p-fluorophenylalanine, DL-o-fluorophenylalanine, and 5-fluorotryptophane. The yddG over-expressing strain also exported more phenylalanine, tyrosine, and tryptophan than normal (Doroshenko et al. (2007) FEMS Microbiol. Lett. 275:312-18).

Tryptophan exporters may be modified in the recombinant bacteria described herein in order to reduce tryptophan export from the cell. Specifically, when the recombinant bacterial cells described herein comprise a genetic modification that reduces export of tryptophan, the bacterial cells retain more tryptophan in the bacterial cell than unmodified bacteria of the same bacterial subtype under the same conditions. Thus, the recombinant bacteria comprising a genetic modification that reduces export of tryptophan may be used to retain more tryptophan in the bacterial cell so that any tryptophan catabolism enzyme expressed in the organism, e.g., co-expressed tryptophan amino transferase, can catabolize the tryptophan.

In one embodiment, the genetic modification is a mutation in an endogenous gene encoding an exporter of tryptophan. In one embodiment, the genetic modification is a mutation in an endogenous gene encoding YddG (see, e.g., SEQ ID NO: 49). In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export tryptophan from the bacterial cell. Assays for testing the activity of an exporter of a tryptophan are well known to one of ordinary skill in the art.

In another embodiment, the genetic modification is a mutation in a promoter of an endogenous gene encoding an exporter of tryptophan.

In yet another embodiment, the genetic modification is an overexpression of a repressor of an exporter of tryptophan. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

8. Methionine

A. Methionine Catabolism Enzymes

In healthy individuals, acquired dietary methionine is catabolized via the trans-sulfuration pathway, where mammalian cells catabolize methionine into homocysteine via S-Adenosyl-methionine and S-Adenosyl-homocysteine. The cystathionine β-synthase (CBS) enzyme then catalyzes the conversion of homocysteine to cystathionine using vitamin B₆ (pyridoxal 5′-phosphate, PLP) as a co-enzyme. Another PLP-dependent enzyme, cystathionine γ-lyase, converts cystathionine into cysteine. Genetic mutations in one or more of these genes can cause metabolic perturbation in the trans-sulfuration pathway that leads to homocystinuria, also known as cystathionine beta synthase deficiency (“CBS deficiency”) (Garland et al., J. Ped. Child Health, 4(8):557-562, 1999). In homocystinuria patients, CBS enzyme deficiency causes elevated levels of homocysteine and low levels of cystathionine in the serum, which leads to excretion of homocysteine into the urine. Inherited homocystinuria, a serious life threatening disease, results in high levels of homocysteine in plasma, tissues and urine. Some of the characteristics of the most common form of homocystinuria are myopia (nearsightedness), displacement of the lens at the front of the eye, higher level of risk of abnormal blood clotting, and fragile bones that are prone to fracture (osteoporosis) or other skeletal irregularities. Homocystinuria may also cause developmental delay/intellectual disability (Mudd et al., Am. J. Hum. Genet., 37:1-31, 1985).

To treat homocystinuria, patients currently receive doses of vitamin B₆ to increase the residual activity of the CBS enzyme and/or restrict intake of dietary methionine to lower the levels of serum homocysteine. Some patients are not responsive to the vitamin B₆ option, while other patients have poor compliance to methionine-restricted diets (Mudd et al., Am. J. Hum. Genet., 37:1-31, 1985). Hence, other options for treating homocystinuria are needed.

Methionine catabolism enzymes may be expressed or modified in the bacteria disclosed herein in order to enhance catabolism of methionine. For example, the genetically engineered bacteria comprising at least one heterologous gene encoding a methionine catabolism enzyme can catabolize methionine to treat a disease associated with methionine, including, but not limited to homocystinuria, cystathionine β-synthase (CBS) deficiency, or cancer, e.g., lymphoblastic leukemia. As used herein, the term “methionine catabolism enzyme” refers to an enzyme involved in the catabolism of methionine. Specifically, when a methionine catabolism enzyme is expressed in a recombinant bacterial cell, the bacterial cell hydrolyzes more methionine into _{L}-homocysteine when the catabolism enzyme is expressed than unmodified bacteria of the same bacterial subtype under the same conditions. In some embodiments, methionine transporters may also be expressed or modified in the recombinant bacteria to enhance methionine import into the cell in order to increase the catabolism of methionine by the methionine catabolism enzyme.

In one embodiment, the methionine catabolism enzyme increases the rate of methionine catabolism in the cell. In one embodiment, the methionine catabolism enzyme decreases the level of methionine in the cell. In another embodiment, the methionine catabolism enzyme increases the level of _{L}-homocysteine in the cell. In one embodiment, the methionine catabolism enzyme increases the level od S-adenosyl-L homocysteine in the cell. In another embodiment, the methionine catabolism enzyme increases the level of L-cystathionine in the cell. In one embodiment, the methionine catabolism enzyme increases the level of 2-oxobutanoate in the cell. In another embodiment, the methionine catabolism enzyme increases the level of _{L}-cysteine in the cell. In one embodiment, the methionine catabolism enzyme increases the level of 3-sulfinoalanine in the cell. In another embodiment, the methionine catabolism enzyme increases the level of 3-sulfinyl-pyruvate in the cell. In one embodiment, the methionine catabolism enzyme increases the level of pyruvate in the cell. In another embodiment, the methionine catabolism enzyme increases the level of sulfite in the cell. In yet another embodiment, the methionine catabolism enzyme increases the level of sulfate in the cell. In one embodiment, the methionine catabolism enzyme increases the level of 2-aminobut-2-enoate in the cell. In another embodiment, the methionine catabolism enzyme increases the level of 4-methylthio-2-oxobutyric acid in the cell. In one embodiment, the methionine catabolism enzyme increases the level of 4-methylthio-2-hydroxybutyric acid in the cell. In another embodiment, the methionine catabolism enzyme increases the level of methional in the cell. In yet another embodiment, the methionine catabolism enzyme increases the level of methionol in the cell.

Methionine catabolism enzymes are well known to those of skill in the art (see, e.g., Huang et al., Mar. Drugs, 13(8):5492-5507, 2015). For example, the adenosylmethionine synthase pathway has been identified in Anabaena cylindrica. In the adenosylmethionine synthase pathway, methionine is catabolized into S-adenosyl-L-homocysteine by an S-adenosylmethionine synthase enzyme, followed by conversion of the S-adenosyl-L-homocysteine into _{L}-homocysteine by an adenosylhomocysteinase enzyme. As another example, two methionine aminotransferase enzymes (including Aro8 and Aro9), and one decarboxylase gene (Aro10) have been identified in Saccharomyces cerevisiae which catabolize methionine (Yin et al. (2015) FEMS Microbiol. Lett. 362(5) pii: fnu043). Methionine aminotransferase enzymes catabolize methionine and 2-oxo carboxylate into 2-oxo-4-methylthiobutanoate and an L-amino acid.

In some embodiments, a methionine catabolism enzyme is encoded by a gene encoding a methionine catabolism enzyme derived from a bacterial species. In some embodiments, a methionine catabolism enzyme is encoded by a gene encoding a methionine catabolism enzyme derived from a non-bacterial species. In some embodiments, a methionine catabolism enzyme is encoded by a gene derived from a eukaryotic species, e.g., a yeast species or a plant species. In one embodiment, the gene encoding the methionine catabolism enzyme is derived from an organism of the genus or species that includes, but is not limited to, Klebsiella quasipneumoniae, Bacillus subtilis, Caenorhabditis elegans, Entamoeba histolytica, Bacillus halodurans, Methylobacterium aquaticum, Saccharomyces cerevisiae, Escherichia coli, and Anabaena cylindrica.

In one embodiment, the at least one methionine catabolism enzyme comprises an S-adenosylmethionine synthase. In another embodiment, the at least one methionine catabolism enzyme comprises an adenosylhomocysteinase. In one embodiment, the at least one methionine catabolism enzyme comprises an S-adenosylmethionine synthase and an adenosylhomocysteinase. In another embodiment, the at least one methionine catabolism enzyme comprises a cystathionine beta-synthase. In one embodiment, the at least one methionine catabolism enzyme comprises a cystathionine gamma-lyase. In one embodiment, the at least one methionine catabolism enzyme comprises a cysteine deoxygenase. In another embodiment, the at least one methionine catabolism enzyme comprises a glutamate oxaloacetate transaminase. In one embodiment, the at let least one methionine catabolism enzyme comprises a sulfite oxidase.

In one embodiment, the methionine catabolism enzyme is an S-adenosylmethionine synthase (E.C. 2.5.1.6). In one embodiment, the S-adenosylmethionine synthase gene is a metK gene. In another embodiment, the S-adenosylmethionine synthase gene is a metK gene from Escherichia coli. In one embodiment, the S-adenosylmethionine synthase gene is from Anabaena cylindrica.

In one embodiment, the S-adenosylmethionine synthase gene has at least about 80% identity with the sequence of SEQ ID NO:50. Accordingly, in one embodiment, the S-adenosylmethionine synthase gene has at least about 90% identity with the sequence of SEQ ID NO:50. Accordingly, in one embodiment, the S-adenosylmethionine synthase gene has at least about 95% identity with the sequence of SEQ ID NO:50. Accordingly, in one embodiment, the S-adenosylmethionine synthase gene has at least about 85%, 86%, 87%, 88%, 89%, 90%, 91%, 92%, 93%, 94%, 95%, 96%, 97%, 98%, or 99% identity with the sequence of SEQ ID NO:50. In another embodiment, the S-adenosylmethionine synthase gene comprises the sequence of SEQ ID NO:50. In yet another embodiment the S-adenosylmethionine synthase gene consists of the sequence of SEQ ID NO:50.

In one embodiment, the methionine catabolism enzyme is an adenosylhomocysteinase (E.C. 3.3.1.1). In one embodiment, the adenosylhomocysteinase gene is an ahcY gene. In another embodiment, the adenosylhomocysteinase gene is an ahcY gene from Anabaena cylindrica.

In one embodiment, the S-adenosylhomocysteinase gene has at least about 80% identity with the sequence of SEQ ID NO:51. Accordingly, in one embodiment, the adenosylhomocysteinase gene has at least about 90% identity with the sequence of SEQ ID NO:51. Accordingly, in one embodiment, the adenosylhomocysteinase gene has at least about 95% identity with the sequence of SEQ ID NO:51. Accordingly, in one embodiment, the adenosylhomocysteinase gene has at least about 85%, 86%, 87%, 88%, 89%, 90%, 91%, 92%, 93%, 94%, 95%, 96%, 97%, 98%, or 99% identity with the sequence of SEQ ID NO:51. In another embodiment, the adenosylhomocysteinase gene comprises the sequence of SEQ ID NO:51. In yet another embodiment the adenosylhomocysteinase gene consists of the sequence of SEQ ID NO:51.

In one embodiment, the methionine catabolism enzyme is an cystathionine beta-synthase (E.C. 4.2.1.22). In one embodiment, the cystathionine beta-synthase gene is a cystathionine beta-synthase gene from Klebsiella quasipneumoniae.

In one embodiment, the cystathionine beta-synthase gene has at least about 80% identity with the sequence of SEQ ID NO:52. Accordingly, in one embodiment, the cystathionine beta-synthase gene has at least about 90% identity with the sequence of SEQ ID NO:52. Accordingly, in one embodiment, the cystathionine beta-synthase gene has at least about 95% identity with the sequence of SEQ ID NO:52. Accordingly, in one embodiment, the cystathionine beta-synthase gene has at least about 85%, 86%, 87%, 88%, 89%, 90%, 91%, 92%, 93%, 94%, 95%, 96%, 97%, 98%, or 99% identity with the sequence of SEQ ID NO:52. In another embodiment, the cystathionine beta-synthase gene comprises the sequence of SEQ ID NO:52. In yet another embodiment the cystathionine beta-synthase gene consists of the sequence of SEQ ID NO:52.

In one embodiment, the methionine catabolism enzyme is an cystathionine gamma-lyase (E.C. 4.4.1.1). In one embodiment, the cystathionine gamma-lyase gene is a cystathionine gamma-lyase gene from Klebsiella pneumoniae.

In one embodiment, the cystathionine gamma-lyase gene has at least about 80% identity with the sequence of SEQ ID NO:53. Accordingly, in one embodiment, the cystathionine gamma-lyase gene has at least about 90% identity with the sequence of SEQ ID NO:53. Accordingly, in one embodiment, the cystathionine gamma-lyase gene has at least about 95% identity with the sequence of SEQ ID NO:53. Accordingly, in one embodiment, the cystathionine gamma-lyase gene has at least about 85%, 86%, 87%, 88%, 89%, 90%, 91%, 92%, 93%, 94%, 95%, 96%, 97%, 98%, or 99% identity with the sequence of SEQ ID NO:53. In another embodiment, the cystathionine gamma-lyase gene comprises the sequence of SEQ ID NO:53. In yet another embodiment the cystathionine gamma-lyase gene consists of the sequence of SEQ ID NO:53.

In one embodiment, the methionine catabolism enzyme is an cysteine dioxygenase (E.C. 1.13.11.20). In one embodiment, the cysteine dioxygenase gene is a cysteine dioxygenase gene from Bacillus subtilis.

In one embodiment, the cysteine dioxygenase gene has at least about 80% identity with the sequence of SEQ ID NO:54. Accordingly, in one embodiment, the cysteine dioxygenase gene has at least about 90% identity with the sequence of SEQ ID NO:54. Accordingly, in one embodiment, the cysteine dioxygenase gene has at least about 95% identity with the sequence of SEQ ID NO:54. Accordingly, in one embodiment, the cysteine dioxygenase gene has at least about 85%, 86%, 87%, 88%, 89%, 90%, 91%, 92%, 93%, 94%, 95%, 96%, 97%, 98%, or 99% identity with the sequence of SEQ ID NO:54. In another embodiment, the cysteine dioxygenase gene comprises the sequence of SEQ ID NO:54. In yet another embodiment the cysteine dioxygenase gene consists of the sequence of SEQ ID NO:54.

In one embodiment, the methionine catabolism enzyme is an glutamate oxaloacetate transaminase (E.C. 2.6.1.1). In one embodiment, the glutamate oxaloacetate transaminase gene is a glutamate oxaloacetate transaminase gene from Caenorhabditis elegans.

In one embodiment, the glutamate oxaloacetate transaminase gene has at least about 80% identity with the sequence of SEQ ID NO:55. Accordingly, in one embodiment, the glutamate oxaloacetate transaminase gene has at least about 90% identity with the sequence of SEQ ID NO:55. Accordingly, in one embodiment, the glutamate oxaloacetate transaminase gene has at least about 95% identity with the sequence of SEQ ID NO:55. Accordingly, in one embodiment, the glutamate oxaloacetate transaminase gene has at least about 85%, 86%, 87%, 88%, 89%, 90%, 91%, 92%, 93%, 94%, 95%, 96%, 97%, 98%, or 99% identity with the sequence of SEQ ID NO:55. In another embodiment, the glutamate oxaloacetate transaminase gene comprises the sequence of SEQ ID NO:55. In yet another embodiment the glutamate oxaloacetate transaminase gene consists of the sequence of SEQ ID NO:55.

In one embodiment, the methionine catabolism enzyme comprises a methionine gamma lyase (E.C. 4.4.1.11). In one embodiment, the methionine gamma lyase gene is a methionine gamma lyase gene from Bacillus halodurans. In one embodiment, the methionine gamma lyase is an Entamoeba histolytica methionine gamma lyase gene.

In one embodiment, the methionine gamma lyase gene has at least about 80% identity with the sequence of SEQ ID NO:56. Accordingly, in one embodiment, the methionine gamma lyase gene has at least about 90% identity with the sequence of SEQ ID NO:56. Accordingly, in one embodiment, the methionine gamma lyase gene has at least about 95% identity with the sequence of SEQ ID NO:56. Accordingly, in one embodiment, the methionine gamma lyase gene has at least about 85%, 86%, 87%, 88%, 89%, 90%, 91%, 92%, 93%, 94%, 95%, 96%, 97%, 98%, or 99% identity with the sequence of SEQ ID NO:56. In another embodiment, the methionine gamma lyase gene comprises the sequence of SEQ ID NO:56. In yet another embodiment the methionine gamma lyase gene consists of the sequence of SEQ ID NO:56.

In another embodiment, the at least one methionine catabolism enzyme comprises a methionine aminotransferase (EC 2.6.1.88). In one embodiment, the methionine aminotransferase gene is a bcaT gene, a KMAT gene, a TyrAT gene, Aro8 gene, Aro9 gene, or a YbdL gene. In another embodiment, the methionine aminotransferase gene is a gene from Saccharomyces cerevisiae, Mycobacterium tuberculosis, Arabidopsis thaliana, Klebsiella pneumonia, or Escherichia coli. In one embodiment, the methionine aminotransferase gene is a Methylyobacterium aquaticum methionine aminotransferase gene.

In one embodiment, the methionine aminotransferase gene has at least about 80% identity with the sequence of SEQ ID NO:57. Accordingly, in one embodiment, the methionine aminotransferase gene has at least about 90% identity with the sequence of SEQ ID NO:57. Accordingly, in one embodiment, the methionine aminotransferase gene has at least about 95% identity with the sequence of SEQ ID NO:57. Accordingly, in one embodiment, the methionine aminotransferase gene has at least about 85%, 86%, 87%, 88%, 89%, 90%, 91%, 92%, 93%, 94%, 95%, 96%, 97%, 98%, or 99% identity with the sequence of SEQ ID NO:57. In another embodiment, the methionine aminotransferase gene comprises the sequence of SEQ ID NO:57. In yet another embodiment the methionine aminotransferase gene consists of the sequence of SEQ ID NO:57.

In one embodiment, the at least one methionine catabolism enzyme comprises a 2-oxo acid decarboxylase. In one embodiment, the 2-oxo acid decarboxylase gene is a Saccharomyces cerevisiae 2-oxo acid decarboxylase gene. In another embodiment, the 2-oxo acid decarboxylase gene is a ARO10 gene from Saccharomyces cerevisiae.

In one embodiment, the ARO10 gene has at least about 80% identity with the sequence of SEQ ID NO:58. Accordingly, in one embodiment, the ARO10 gene has at least about 90% identity with the sequence of SEQ ID NO:58. Accordingly, in one embodiment, the ARO10 gene has at least about 95% identity with the sequence of SEQ ID NO:58. Accordingly, in one embodiment, the ARO10 gene has at least about 85%, 86%, 87%, 88%, 89%, 90%, 91%, 92%, 93%, 94%, 95%, 96%, 97%, 98%, or 99% identity with the sequence of SEQ ID NO:58. In another embodiment, the ARO10 gene comprises the sequence of SEQ ID NO:58. In yet another embodiment the ARO10 gene consists of the sequence of SEQ ID NO:58.

The present disclosure further comprises genes encoding functional fragments of a methionine amino acid catabolism enzyme or functional variants of a methionine amino acid catabolism enzyme.

Assays for testing the activity of a methionine catabolism enzyme, a methionine catabolism enzyme functional variant, or a methionine catabolism enzyme functional fragment are well known to one of ordinary skill in the art. For example, methionine catabolism can be assessed by expressing the protein, functional variant, or fragment thereof, in a recombinant bacterial cell that lacks endogenous methionine catabolism enzyme activity. Other methods are also well known to one of ordinary skill in the art (see, e.g., Dolzan et al., FEBS Letters, 574:141-146, 2004, the entire contents of which are incorporated by reference).

In one embodiment, the bacterial cell comprises a heterologous gene encoding a methionine catabolism enzyme. In one embodiment, the bacterial cell comprises a heterologous gene encoding a transporter of methionine and a heterologous gene encoding a methionine catabolism enzyme. In one embodiment, the bacterial cell comprises a heterologous gene encoding a methionine catabolism enzyme and a genetic modification that reduces export of methionine. In one embodiment, the bacterial cell comprises a heterologous gene encoding a transporter of methionine, a heterologous gene encoding a methionine catabolism enzyme, and a genetic modification that reduces export of methionine. Transporters and exporters are described in more detail in the subsections, below.

B. Transporters of Methionine

Methionine transporters may be expressed or modified in the recombinant bacteria described herein in order to enhance methionine transport into the cell. Specifically, when the transporter of methionine is expressed in the recombinant bacterial cells described herein, the bacterial cells import more methionine into the cell when the transporter is expressed than unmodified bacteria of the same bacterial subtype under the same conditions. Thus, the genetically engineered bacteria comprising a heterologous gene encoding transporter of methionine which may be used to import methionine into the bacteria so that any gene encoding a methionine catabolism enzyme expressed in the organism can catabolize the methionine to treat a disease associated with methionine, such as cancer.

The uptake of methionine into bacterial cells is mediated by proteins well known to those of skill in the art. For example, a methionine transporter operon has been identified in Corynebacterium glutamicum (Trotschel et al., J. Bacteriology, 187(11):3786-3794, 2005). In addition, the high affinity MetD ABC transporter system has been characterized in Escherichia coli (Kadaba et al. (2008) Science 5886: 250-253; Kadner and Watson (1974) J. Bacteriol. 119: 401-9). The MetD transporter system is capable of mediating the translocation of several substrates across the bacterial membrane, including methionine. The metD system of Escherichia coli consists of MetN (encoded by metN), which comprises the ATPase domain, MetI (encoded by metI), which comprises the transmembrane domain, and MetQ (encoded by metQ), the cognate binding protein which is located in the periplasm. Orthologues of the genes encoding the E. coli metD transporter system have been identified in multiple organisms including, e.g., Yersinia pestis, Vibrio cholerae, Pasteurella multocida, Haemophilus influenza, Agrobacterium tumefaciens, Sinorhizobium meliloti, Brucella meliloti, and Mesorhizobium loti (Merlin et al. (2002) J. Bacteriol. 184: 5513-7).

In one embodiment, the at least one gene encoding a transporter of methionine is a metP gene, a metN gene, a metI gene, or a metQ gene from Corynebacterium glutamicum, Escherichia coli, and Bacillus subtilis (Trotschel et al., J. Bacteriology, 187(11):3786-3794, 2005).

In one embodiment, the metP gene has at least about 80% identity with the sequence of SEQ ID NO:59. Accordingly, in one embodiment, the metP gene has at least about 90% identity with the sequence of SEQ ID NO:59. Accordingly, in one embodiment, the metP gene has at least about 95% identity with the sequence of SEQ ID NO:59. Accordingly, in one embodiment, the metP gene has at least about 85%, 86%, 87%, 88%, 89%, 90%, 91%, 92%, 93%, 94%, 95%, 96%, 97%, 98%, or 99% identity with the sequence of SEQ ID NO:59. In another embodiment, the metP gene comprises the sequence of SEQ ID NO:59. In yet another embodiment the metP gene consists of the sequence of SEQ ID NO:59.

In one embodiment, the metN gene has at least about 80% identity with the sequence of SEQ ID NO:60. Accordingly, in one embodiment, the metN gene has at least about 90% identity with the sequence of SEQ ID NO:60. Accordingly, in one embodiment, the metN gene has at least about 95% identity with the sequence of SEQ ID NO:60. Accordingly, in one embodiment, the metN gene has at least about 85%, 86%, 87%, 88%, 89%, 90%, 91%, 92%, 93%, 94%, 95%, 96%, 97%, 98%, or 99% identity with the sequence of SEQ ID NO:60. In another embodiment, the metN gene comprises the sequence of SEQ ID NO:60. In yet another embodiment the metN gene consists of the sequence of SEQ ID NO:60.

In one embodiment, the metI gene has at least about 80% identity with the sequence of SEQ ID NO:61. Accordingly, in one embodiment, the metI gene has at least about 90% identity with the sequence of SEQ ID NO:61. Accordingly, in one embodiment, the metI gene has at least about 95% identity with the sequence of SEQ ID NO:61. Accordingly, in one embodiment, the metI gene has at least about 85%, 86%, 87%, 88%, 89%, 90%, 91%, 92%, 93%, 94%, 95%, 96%, 97%, 98%, or 99% identity with the sequence of SEQ ID NO:61. In another embodiment, the metI gene comprises the sequence of SEQ ID NO:61. In yet another embodiment the metI gene consists of the sequence of SEQ ID NO:61.

In one embodiment, the metQ gene has at least about 80% identity with the sequence of SEQ ID NO:62. Accordingly, in one embodiment, the metQ gene has at least about 90% identity with the sequence of SEQ ID NO:62. Accordingly, in one embodiment, the metQ gene has at least about 95% identity with the sequence of SEQ ID NO:62. Accordingly, in one embodiment, the metQ gene has at least about 85%, 86%, 87%, 88%, 89%, 90%, 91%, 92%, 93%, 94%, 95%, 96%, 97%, 98%, or 99% identity with the sequence of SEQ ID NO:62. In another embodiment, the metQ gene comprises the sequence of SEQ ID NO:62. In yet another embodiment the metQ gene consists of the sequence of SEQ ID NO:62.

In some embodiments, the transporter of methionine is encoded by a transporter of methionine gene derived from a bacterial genus or species, including but not limited to, Corynebacterium glutamicum, Escherichia coli, and Bacillus subtilis. In some embodiments, the bacterial species is Escherichia coli strain Nissle.

Assays for testing the activity of a transporter of methionine, a functional variant of a transporter of methionine, or a functional fragment of transporter of methionine are well known to one of ordinary skill in the art. For example, import of methionine may be determined using the methods as described in Trotschel et al., J. Bacteriology, 187(11):3786-3794, 2005, the entire contents of which are expressly incorporated by reference herein.

In one embodiment, when the transporter of a methionine is expressed in the recombinant bacterial cells described herein, the bacterial cells import 10% more methionine into the bacterial cell when the transporter is expressed than unmodified bacteria of the same bacterial subtype under the same conditions. In another embodiment, when the transporter of methionine is expressed in the recombinant bacterial cells described herein, the bacterial cells import 20%, 30%, 40%, 50%, 60%, 70%, 80%, 90% or 100% more methionine into the bacterial cell when the transporter is expressed than unmodified bacteria of the same bacterial subtype under the same conditions. In yet another embodiment, when the transporter of methionine is expressed in the recombinant bacterial cells described herein, the bacterial cells import two-fold more methionine into the cell when the transporter is expressed than unmodified bacteria of the same bacterial subtype under the same conditions. In yet another embodiment, when the transporter of methionine is expressed in the recombinant bacterial cells described herein, the bacterial cells import three-fold, four-fold, five-fold, six-fold, seven-fold, eight-fold, nine-fold, or ten-fold more methionine into the cell when the transporter is expressed than unmodified bacteria of the same bacterial subtype under the same conditions.

C. Exporters of Methionine

Methionine exporters may be modified in the recombinant bacteria described herein in order to reduce methionine export from the cell. Specifically, when the recombinant bacterial cells described herein comprise a genetic modification that reduces export of methionine, the bacterial cells retain more methionine in the bacterial cell than unmodified bacteria of the same bacterial subtype under the same conditions. Thus, the recombinant bacteria comprising a genetic modification that reduces export of methionine may be used to retain more methionine in the bacterial cell so that any methionine catabolism enzyme expressed in the organism, e.g., co-expressed methionine catabolism enzyme, can catabolize the methionine.

Exporters of methionine are well known to one of ordinary skill in the art. For example, the MetE methionine exporter from Bacillus atrophaeus, and the BrnFE methionine exporter from Corynebacterium glutamicum have been described (Trotschel et al., J. Bacteriology, 187(11):3786-3794, 2005).

In one embodiment, the metE gene has at least about 80% identity with the sequence of SEQ ID NO:63. Accordingly, in one embodiment, the metE gene has at least about 90% identity with the sequence of SEQ ID NO:63. Accordingly, in one embodiment, the metE gene has at least about 95% identity with the sequence of SEQ ID NO:63. Accordingly, in one embodiment, the metE gene has at least about 85%, 86%, 87%, 88%, 89%, 90%, 91%, 92%, 93%, 94%, 95%, 96%, 97%, 98%, or 99% identity with the sequence of SEQ ID NO:63. In another embodiment, the metE gene comprises the sequence of SEQ ID NO:63. In yet another embodiment the metE gene consists of the sequence of SEQ ID NO:63.

In one embodiment, the brnF gene has at least about 80% identity with the sequence of SEQ ID NO:64. Accordingly, in one embodiment, the brnF gene has at least about 90% identity with the sequence of SEQ ID NO:64. Accordingly, in one embodiment, the brnF gene has at least about 95% identity with the sequence of SEQ ID NO:64. Accordingly, in one embodiment, the brnF gene has at least about 85%, 86%, 87%, 88%, 89%, 90%, 91%, 92%, 93%, 94%, 95%, 96%, 97%, 98%, or 99% identity with the sequence of SEQ ID NO:64. In another embodiment, the brnF gene comprises the sequence of SEQ ID NO:64. In yet another embodiment the brnF gene consists of the sequence of SEQ ID NO:64.

In one embodiment, the brnE gene has at least about 80% identity with the sequence of SEQ ID NO:65. Accordingly, in one embodiment, the brnE gene has at least about 90% identity with the sequence of SEQ ID NO:65. Accordingly, in one embodiment, the brnE gene has at least about 95% identity with the sequence of SEQ ID NO:65. Accordingly, in one embodiment, the brnE gene has at least about 85%, 86%, 87%, 88%, 89%, 90%, 91%, 92%, 93%, 94%, 95%, 96%, 97%, 98%, or 99% identity with the sequence of SEQ ID NO:65. In another embodiment, the brnE gene comprises the sequence of SEQ ID NO:65. In yet another embodiment the brnE gene consists of the sequence of SEQ ID NO:65.

In one embodiment, the genetic modification is a mutation in an endogenous gene encoding an exporter of methionine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export methionine from the bacterial cell.

In another embodiment, the genetic modification is a mutation in a promoter of an endogenous gene encoding an exporter of methionine.

In yet another embodiment, the genetic modification is an overexpression of a repressor of an exporter of methionine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

9. Threonine

A. Threonine Catabolism Enzymes

Threonine catabolism enzymes may be expressed or modified in the bacteria disclosed herein in order to enhance catabolism of threonine. As used herein, the term “threonine catabolism enzyme” refers to an enzyme involved in the catabolism of threonine. Specifically, when a threonine catabolism enzyme is expressed in a recombinant bacterial cell, the bacterial cell hydrolyzes more threonine into glycine or amino-ketobutyrate when the catabolism enzyme is expressed than unmodified bacteria of the same bacterial subtype under the same conditions. Thus, the genetically engineered bacteria comprising a heterologous gene encoding a threonine catabolism enzyme can catabolize threonine to treat a disease associated with threonine, such as cancer, e.g., non-small cell lung cancer.

In one embodiment, the threonine catabolism enzyme increases the rate of threonine catabolism in the cell. In one embodiment, the threonine catabolism enzyme decreases the level of threonine in the cell. In another embodiment, the threonine catabolism enzyme increases the level of glycine in the cell. In another embodiment, threonine catabolism enzyme increases the level of amino-ketobutyrate in the cell.

Threonine catabolism enzymes are well known to those of skill in the art (see, e.g., Simic et al., Applied and Environmental Microbiology, 68(7):3321-3327, 2002). In bacteria and plants, threonine dehydrogenase is capable of converting threonine into amino-ketobutyrate, with subsequent conversion of 2-amino-3-ketobutyrate by amino-keto-butyrate lyase (AKB-CoA lyase) or spontaneous decarboxylation of 2-amino-3-ketobutyrate. Threonine is also converted into glycine and acetaldehyde by serine hydroxymethyltransferase (SMHT).

In some embodiments, a threonine catabolism enzyme is encoded by a gene encoding a threonine catabolism enzyme derived from a bacterial species. In some embodiments, a threonine catabolism enzyme is encoded by a gene encoding a threonine catabolism enzyme derived from a non-bacterial species. In some embodiments, a threonine catabolism enzyme is encoded by a gene derived from a eukaryotic species, e.g., a yeast species or a plant species. In one embodiment, the gene encoding the threonine catabolism enzyme is derived from an organism of the genus or species that includes, but is not limited to, Corynebacterium glutamicum, Salmonella enterica, and Escherichia coli.

In one embodiment, the threonine catabolism enzyme is a serine hydroxymethyltransferase (SHMT). As used herein, “serine hydroxymethyltransferase” refers to any polypeptide having enzymatic activity that catalyzes the conversion of threonine to glycine and acetaldehyde. For example, the SHMT enzyme of Corynebacterium glutamicum (encoded by the glyA gene) is capable of metabolizing threonine (see, e.g., Simic et al., Applied and Environmental Microbiology, 68(7):3321-3327, 2002). SHMT may also convert serine to glycine. Other distinct serine hydroxymethyltransferase enzymes are also known in the art. In some embodiments, an SHMT enzyme is co-expressed with an AKB-CoA lyase.

In one embodiment, the SHMT gene is a glyA gene. In another embodiment, the glyA gene is a Corynebacterium glutamicum glyA gene. In another embodiment, the glyA gene is a Escherichia coli glyA gene.

In one embodiment, the glyA gene has at least about 80% identity with the sequence of SEQ ID NO:68. Accordingly, in one embodiment, the glyA gene has at least about 90% identity with the sequence of SEQ ID NO:68. Accordingly, in one embodiment, the glyA gene has at least about 95% identity with the sequence of SEQ ID NO:68. Accordingly, in one embodiment, the glyA gene has at least about 85%, 86%, 87%, 88%, 89%, 90%, 91%, 92%, 93%, 94%, 95%, 96%, 97%, 98%, or 99% identity with the sequence of SEQ ID NO:68. In another embodiment, the glyA gene comprises the sequence of SEQ ID NO:68. In yet another embodiment the glyA gene consists of the sequence of SEQ ID NO:68.

In another embodiment, the threonine catabolism enzyme is a threonine dehydrogenase (EC 1.1.1.103). As used herein, “threonine dehydrogenase” refers to any polypeptide having enzymatic activity that catalyzes the conversion of threonine to amino-ketobutyrate. For example, the threonine dehydrogenase enzyme of Escherichia coli (encoded by the tdh gene) is capable of metabolizing threonine (see, e.g., Simic et al., Applied and Environmental Microbiology, 68(7):3321-3327, 2002). Other distinct threonine dehydrogenase enzymes are also known in the art.

In one embodiment, the threonine dehydrogenase gene is a tdh gene. In another embodiment, the tdh gene is an Escherichia coli tdh gene. In another embodiment, the tdh gene is an Salmonella enterica tdh gene.

In one embodiment, the tdh gene has at least about 80% identity with the sequence of SEQ ID NO:66. Accordingly, in one embodiment, the tdh gene has at least about 90% identity with the sequence of SEQ ID NO:66. Accordingly, in one embodiment, the tdh gene has at least about 95% identity with the sequence of SEQ ID NO:66. Accordingly, in one embodiment, the tdh gene has at least about 85%, 86%, 87%, 88%, 89%, 90%, 91%, 92%, 93%, 94%, 95%, 96%, 97%, 98%, or 99% identity with the sequence of SEQ ID NO:66. In another embodiment, the tdh gene comprises the sequence of SEQ ID NO:66. In yet another embodiment the tdh gene consists of the sequence of SEQ ID NO:66.

In another embodiment, the threonine catabolism enzyme is a threonine aldolase (EC 4.1.2.5). As used herein, “threonine aldolase” refers to any polypeptide having enzymatic activity that catalyzes the conversion of threonine to glycine and acetaldehyde. For example, the threonine aldolase enzyme of Escherichia coli (encoded by the ltaE gene) is capable of metabolizing threonine (see, e.g., di Salvo et al. (2014) FEBS J. 281(1): 129-145). Other distinct threonine aldolase enzymes are also known in the art.

In one embodiment, the threonine aldolase gene is a ltaE gene. In another embodiment, the ltaE gene is an Escherichia coli ltaE gene.

In one embodiment, the ltaE gene has at least about 80% identity with the sequence of SEQ ID NO:67. Accordingly, in one embodiment, the ltaE gene has at least about 90% identity with the sequence of SEQ ID NO:67. Accordingly, in one embodiment, the ltaE gene has at least about 95% identity with the sequence of SEQ ID NO:67. Accordingly, in one embodiment, the ltaE gene has at least about 85%, 86%, 87%, 88%, 89%, 90%, 91%, 92%, 93%, 94%, 95%, 96%, 97%, 98%, or 99% identity with the sequence of SEQ ID NO:67. In another embodiment, the ltaE gene comprises the sequence of SEQ ID NO:67. In yet another embodiment the ltaE gene consists of the sequence of SEQ ID NO:67.

The present disclosure further comprises genes encoding functional fragments of a threonine amino acid catabolism enzyme or functional variants of a threonine amino acid catabolism enzyme.

Assays for testing the activity of a threonine catabolism enzyme, a threonine catabolism enzyme functional variant, or a threonine catabolism enzyme functional fragment are well known to one of ordinary skill in the art. For example, threonine catabolism can be assessed by expressing the protein, functional variant, or fragment thereof, in a recombinant bacterial cell that lacks endogenous threonine catabolism enzyme activity. Threonine catabolism can be assessed by measuring the conversion of threonine to amino-ketobutyrate (see, e.g., Simic et al., Applied and Environmental Microbiology, 68(7):3321-3327, 2002), the entire contents of which are incorporated by reference).

In another embodiment, the gene encoding the threonine catabolism enzyme is co-expressed with an additional threonine catabolism enzyme, for example, a SHMT enzyme is co-expressed with a threonine dehydrogenase enzyme.

In one embodiment, the bacterial cell comprises a heterologous gene encoding a threonine catabolism enzyme. In one embodiment, the bacterial cell comprises a heterologous gene encoding a transporter of threonine and a heterologous gene encoding a threonine catabolism enzyme. In one embodiment, the bacterial cell comprises a heterologous gene encoding a threonine catabolism enzyme and a genetic modification that reduces export of threonine. In one embodiment, the bacterial cell comprises a heterologous gene encoding a transporter of threonine, a heterologous gene encoding a threonine catabolism enzyme, and a genetic modification that reduces export of threonine. Transporters and exporters are described in more detail in the subsections, below.

B. Transporters of Threonine

Threonine transporters may be expressed or modified in the recombinant bacteria described herein in order to enhance threonine transport into the cell. Specifically, when the transporter of threonine is expressed in the recombinant bacterial cells described herein, the bacterial cells import more threonine into the cell when the transporter is expressed than unmodified bacteria of the same bacterial subtype under the same conditions. Thus, the genetically engineered bacteria comprising a heterologous gene encoding transporter of threonine which may be used to import threonine into the bacteria so that any gene encoding a threonine catabolism enzyme expressed in the organism can catabolize the threonine to treat a disease associated with threonine, such as cancer.

The uptake of threonine into bacterial cells is mediated by proteins well known to those of skill in the art. For example, a threonine transporter TdcC has been identified (Wook Lee et al., Nature Chemical Biology, 8:536-546, 2012). Additional serine/threonine transporters have been identified and are disclosed in the serine section herein.

In one embodiment, the at least one gene encoding a transporter of threonine is the tdcC gene. In one embodiment, the bacterial cell described herein has been genetically engineered to comprise at least one heterologous tdcC gene. In one embodiment, the at least one gene encoding a transporter of threonine is the Escherichia coli tdcC gene. In one embodiment, the at least one gene encoding a transporter of threonine is the Salmonella typhimurium tdcC gene.

In one embodiment, the tdcC gene has at least about 80% identity with the sequence of SEQ ID NO:69. Accordingly, in one embodiment, the tdcC gene has at least about 90% identity with the sequence of SEQ ID NO:69. Accordingly, in one embodiment, the tdcC gene has at least about 95% identity with the sequence of SEQ ID NO:69. Accordingly, in one embodiment, the tdcC gene has at least about 85%, 86%, 87%, 88%, 89%, 90%, 91%, 92%, 93%, 94%, 95%, 96%, 97%, 98%, or 99% identity with the sequence of SEQ ID NO:69. In another embodiment, the tdcC gene comprises the sequence of SEQ ID NO:69. In yet another embodiment the tdcC gene consists of the sequence of SEQ ID NO:69.

In some embodiments, the transporter of threonine is encoded by a transporter of threonine gene derived from a bacterial genus or species, including but not limited to, Escherichia coli or Salmonella typhimurium. In some embodiments, the bacterial species is Escherichia coli. In some embodiments, the bacterial species is Escherichia coli strain Nissle.

Assays for testing the activity of a transporter of threonine, a functional variant of a transporter of threonine, or a functional fragment of transporter of threonine are well known to one of ordinary skill in the art. For example, import of threonine may be determined using the methods as described in Wook Lee et al., Nature Chemical Biology, 8:536-546, 2012, the entire contents of which are expressly incorporated by reference herein.

In one embodiment, when the transporter of a threonine is expressed in the recombinant bacterial cells described herein, the bacterial cells import 10% more threonine into the bacterial cell when the transporter is expressed than unmodified bacteria of the same bacterial subtype under the same conditions. In another embodiment, when the transporter of threonine is expressed in the recombinant bacterial cells described herein, the bacterial cells import 20%, 30%, 40%, 50%, 60%, 70%, 80%, 90% or 100% more threonine into the bacterial cell when the transporter is expressed than unmodified bacteria of the same bacterial subtype under the same conditions. In yet another embodiment, when the transporter of threonine is expressed in the recombinant bacterial cells described herein, the bacterial cells import two-fold more threonine into the cell when the transporter is expressed than unmodified bacteria of the same bacterial subtype under the same conditions. In yet another embodiment, when the transporter of threonine is expressed in the recombinant bacterial cells described herein, the bacterial cells import three-fold, four-fold, five-fold, six-fold, seven-fold, eight-fold, nine-fold, or ten-fold more threonine into the cell when the transporter is expressed than unmodified bacteria of the same bacterial subtype under the same conditions.

C. Exporters of Threonine

Threonine exporters may be modified in the recombinant bacteria described herein in order to reduce threonine export from the cell. Specifically, when the recombinant bacterial cells described herein comprise a genetic modification that reduces export of threonine, the bacterial cells retain more threonine in the bacterial cell than unmodified bacteria of the same bacterial subtype under the same conditions. Thus, the recombinant bacteria comprising a genetic modification that reduces export of threonine may be used to retain more threonine in the bacterial cell so that any threonine catabolism enzyme expressed in the organism, e.g., co-expressed threonine catabolism enzyme, can catabolize the threonine.

In one embodiment, the genetic modification is a mutation in an endogenous gene encoding an exporter of threonine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export threonine from the bacterial cell.

Multiple threonine exporters are known in the art. For example, the rhtA gene (SEQ ID NO: 70) encodes an exporter of threonine (Livshits et al., Res. Microbiol., 154(2):123-135, 2003). The rhtB (SEQ ID NO: 71) and rhtC (SEQ ID NO: 72) genes also encode threonine exporters (Wook Lee et al., Nature Chemical Biology, 8:536-546, 2012). Additional serine/threonine exporters have been identified and are disclosed in the serine section herein. Assays for testing the activity of an exporter of a threonine are well known to one of ordinary skill in the art (Wook Lee et al., Nature Chemical Biology, 8:536-546, 2012).

In another embodiment, the genetic modification is a mutation in a promoter of an endogenous gene encoding an exporter of threonine.

In yet another embodiment, the genetic modification is an overexpression of a repressor of an exporter of threonine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

10. Cysteine

A. Cysteine Catabolism Enzymes

Cysteine catabolism enzymes may be expressed or modified in the bacteria disclosed herein in order to enhance catabolism of cysteine. As used herein, the term “cysteine catabolism enzyme” refers to an enzyme involved in the catabolism of cysteine. Specifically, when a cysteine catabolism enzyme is expressed in a recombinant bacterial cell, the bacterial cell catabolizes more cysteine when the catabolism enzyme is expressed than unmodified bacteria of the same bacterial subtype under the same conditions. Thus, the genetically engineered bacteria comprising a heterologous gene encoding a cysteine catabolism enzyme can catabolize cysteine to treat a disease associated with cysteine, such as cancer, e.g., lymphoblastic lymphoma.

In one embodiment, the cysteine catabolism enzyme increases the rate of cysteine catabolism in the cell. In one embodiment, the cysteine catabolism enzyme decreases the level of cysteine in the cell. In another embodiment, the cysteine catabolism enzyme increases the level of hydrogen sulfide in the cell. In another embodiment, the cysteine catabolism enzyme increases the level of ammonia in the cell. In yet another embodiment, the cysteine catabolism enzyme increases the level of pyruvate in the cell. In one embodiment, the cysteine catabolism enzyme increases the level of glutamate in the cell. In another embodiment, the cysteine catabolism enzyme increases the level of 3-mercaptopyruvate in the cell. In one embodiment, the cysteine catabolism enzyme increases the level of cystathionine in the cell. In another embodiment, the cysteine catabolism enzyme increases the level of serine in the cell.

Cysteine catabolism enzymes are well known to those of skill in the art (see, e.g., Carbonero et al. (2012) Front Physiol. 3: 448; Quazi and Aitken (2009) Biochim. Biophys Acta. 1794(6): 892-7; and Shatalin et al. (2011) Science 334: 986-90). For example, cysteine desulfhydrase is capable of converting cysteine into hydrogen sulfide. Cysteine is also converted into cystathionine or into hydrogen sulfide by cystathionine β-synthase (CBS). Cystathionine γ-lyase (CSE) is also capable of catalyzing the conversion of cysteine to hydrogen sulfide. Finally, 3-mercaptopyruvate sulfurtransferase (3MST) is also capable of catalyzing the conversion of cysteine to hydrogen sulfide via the intermediate synthesis of 3-mercaptopyruvate produced by cysteine aminotransferase.

In some embodiments, a cysteine catabolism enzyme is encoded by a gene encoding a cysteine catabolism enzyme derived from a bacterial species. In some embodiments, a cysteine catabolism enzyme is encoded by a gene encoding a cysteine catabolism enzyme derived from a non-bacterial species. In some embodiments, a cysteine catabolism enzyme is encoded by a gene derived from a eukaryotic species, e.g., a yeast species, a mammalian species or a plant species. In one embodiment, the gene encoding the cysteine catabolism enzyme is derived from an organism of the genus or species that includes, but is not limited to, Streptococcus, Prevotella, Fusobacterium, Clostridium, Bacillus, Enterobacter, Escherichia, Klebsiella, Desulfovibrio, Helicobacter, Lactobacillus, Leishmania, Pseudomonas, Salmonella, Staphylococcus, Trypanosoma, Mycobacterium, Desulfovibrio desulfuricans, Escherichia coli, Trypanosoma grayi, Helicobacter pylori, Bacillus anthracia, Leishmania major, Pseudomonas aeruginosa, Salmonella typhimurium, Mycobacterium tuberculosis, and Staphylococcus aureus.

In one embodiment, the cysteine catabolism enzyme is a cysteine desulfhydrase. As used herein, “cysteine desulfhydrase” refers to any polypeptide having enzymatic activity that catalyzes the conversion of cysteine to hydrogen sulfide. Many cysteine desulfhydrases are known in the art (see, e.g., Carbonero et al. (2012) Front Physiol. 3: 448; Awano et al. (2005) Appl. Environ. Microbiol. 71(7): 4149-52).

In one embodiment, the cysteine desulfhydrase gene is a dcyD gene. In another embodiment, the desulfhydrase gene is a Escherichia coli dcyD gene.

In one embodiment, the dcyD gene has at least about 80% identity with the sequence of SEQ ID NO:73. Accordingly, in one embodiment, the dcyD gene has at least about 90% identity with the sequence of SEQ ID NO:73. Accordingly, in one embodiment, the dcyD gene has at least about 95% identity with the sequence of SEQ ID NO:73. Accordingly, in one embodiment, the dcyD gene has at least about 85%, 86%, 87%, 88%, 89%, 90%, 91%, 92%, 93%, 94%, 95%, 96%, 97%, 98%, or 99% identity with the sequence of SEQ ID NO:73. In another embodiment, the dcyD gene comprises the sequence of SEQ ID NO:73. In yet another embodiment the dcyD gene consists of the sequence of SEQ ID NO:73.

In one embodiment, the cysteine desulfhydrase gene is a tnaA gene. In another embodiment, the desulfhydrase gene is a Escherichia coli tnaA gene.

In one embodiment, the tnaA gene has at least about 80% identity with the sequence of SEQ ID NO:74. Accordingly, in one embodiment, the tnaA gene has at least about 90% identity with the sequence of SEQ ID NO:74. Accordingly, in one embodiment, the tnaA gene has at least about 95% identity with the sequence of SEQ ID NO:74. Accordingly, in one embodiment, the tnaA gene has at least about 85%, 86%, 87%, 88%, 89%, 90%, 91%, 92%, 93%, 94%, 95%, 96%, 97%, 98%, or 99% identity with the sequence of SEQ ID NO:74. In another embodiment, the tnaA gene comprises the sequence of SEQ ID NO:74. In yet another embodiment the tnaA gene consists of the sequence of SEQ ID NO:74.

In one embodiment, the cysteine desulfhydrase gene is a cysK gene. In another embodiment, the desulfhydrase gene is a Escherichia coli cysK gene.

In one embodiment, the cysK gene has at least about 80% identity with the sequence of SEQ ID NO:75. Accordingly, in one embodiment, the cysK gene has at least about 90% identity with the sequence of SEQ ID NO:75. Accordingly, in one embodiment, the cysK gene has at least about 95% identity with the sequence of SEQ ID NO:75. Accordingly, in one embodiment, the cysK gene has at least about 85%, 86%, 87%, 88%, 89%, 90%, 91%, 92%, 93%, 94%, 95%, 96%, 97%, 98%, or 99% identity with the sequence of SEQ ID NO:75. In another embodiment, the cysK gene comprises the sequence of SEQ ID NO:75. In yet another embodiment the cysK gene consists of the sequence of SEQ ID NO:75.

In one embodiment, the cysteine desulfhydrase gene is a cysM gene. In another embodiment, the desulfhydrase gene is a Escherichia coli cysM gene.

In one embodiment, the cysM gene has at least about 80% identity with the sequence of SEQ ID NO:76. Accordingly, in one embodiment, the cysM gene has at least about 90% identity with the sequence of SEQ ID NO:76. Accordingly, in one embodiment, the cysM gene has at least about 95% identity with the sequence of SEQ ID NO:76. Accordingly, in one embodiment, the cysM gene has at least about 85%, 86%, 87%, 88%, 89%, 90%, 91%, 92%, 93%, 94%, 95%, 96%, 97%, 98%, or 99% identity with the sequence of SEQ ID NO:76. In another embodiment, the cysM gene comprises the sequence of SEQ ID NO:76. In yet another embodiment the cysM gene consists of the sequence of SEQ ID NO:76.

In one embodiment, the cysteine desulfhydrase gene is a malY gene. In another embodiment, the desulfhydrase gene is a Escherichia coli malY gene.

In one embodiment, the malY gene has at least about 80% identity with the sequence of SEQ ID NO:77. Accordingly, in one embodiment, the malY gene has at least about 90% identity with the sequence of SEQ ID NO:77. Accordingly, in one embodiment, the malY gene has at least about 95% identity with the sequence of SEQ ID NO:77. Accordingly, in one embodiment, the malY gene has at least about 85%, 86%, 87%, 88%, 89%, 90%, 91%, 92%, 93%, 94%, 95%, 96%, 97%, 98%, or 99% identity with the sequence of SEQ ID NO:77. In another embodiment, the malY gene comprises the sequence of SEQ ID NO:77. In yet another embodiment the malY gene consists of the sequence of SEQ ID NO:77.

In another embodiment, the cysteine catabolism enzyme is a cystathionine (3-synthase. As used herein, “cystathionine β-synthase” refers to any polypeptide having enzymatic activity that catalyzes the condensation of cysteine and homocysteine to form cystathionine and hydrogen sulfide. Many distinct cystathionine β-synthase enzymes are also known in the art (see, e.g., Shatalin et al. (2011)).

In one embodiment, the cystathionine β-synthase gene is a CBS gene. In one embodiment, the cystathionine β-synthase gene is a Bacillus anthracia CBS gene. In one embodiment, the CBS gene is a Pseudomonas aeruginosa CBS gene. In one embodiment, the cystathionine β-synthase gene is a Staphylococcus aureus CBS gene. In one embodiment, the cystathionine β-synthase gene is a Helicobacter pylori CBS gene.

In one embodiment, the CBS gene has at least about 80% identity with the sequence of SEQ ID NO:80. Accordingly, in one embodiment, the CBS gene has at least about 90% identity with the sequence of SEQ ID NO:80. Accordingly, in one embodiment, the CBS gene has at least about 95% identity with the sequence of SEQ ID NO:80. Accordingly, in one embodiment, the CBS gene has at least about 85%, 86%, 87%, 88%, 89%, 90%, 91%, 92%, 93%, 94%, 95%, 96%, 97%, 98%, or 99% identity with the sequence of SEQ ID NO:80. In another embodiment, the CBS gene comprises the sequence of SEQ ID NO:80. In yet another embodiment the CBS gene consists of the sequence of SEQ ID NO:80.

In another embodiment, the cysteine catabolism enzyme is a cystathionine γ-lyase. As used herein, “cystathionine γ-lyase” refers to any polypeptide having enzymatic activity that catalyzes the catabolism of cysteine to produce hydrogen sulfide. Many distinct cystathionine γ-lyase enzymes are also known in the art (see, e.g., Shatalin et al. (2011)).

In one embodiment, the cystathionine β-synthase gene is a CSE gene. In one embodiment, the cystathionine γ-lyase gene is a Bacillus anthracia CSE gene. In one embodiment, the CBS gene is a Pseudomonas aeruginosa CSE gene. In one embodiment, the cystathionine β-synthase gene is a Staphylococcus aureus CSE gene. In one embodiment, the cystathionine β-synthase gene is a Helicobacter pylori CSE gene. In one embodiment, the cystathionine β-synthase gene is a Trypanosoma grayi CSE gene.

In one embodiment, the CSE gene has at least about 80% identity with the sequence of SEQ ID NO:79. Accordingly, in one embodiment, the CSE gene has at least about 90% identity with the sequence of SEQ ID NO:79. Accordingly, in one embodiment, the CSE gene has at least about 95% identity with the sequence of SEQ ID NO:79. Accordingly, in one embodiment, the CSE gene has at least about 85%, 86%, 87%, 88%, 89%, 90%, 91%, 92%, 93%, 94%, 95%, 96%, 97%, 98%, or 99% identity with the sequence of SEQ ID NO:79. In another embodiment, the CSE gene comprises the sequence of SEQ ID NO:79. In yet another embodiment the CSE gene consists of the sequence of SEQ ID NO:79.

In another embodiment, the cysteine catabolism enzyme is a cysteine aminotransferase (also known as a cysteine transaminase; CAT). As used herein, “cysteine aminotransferase” refers to any polypeptide having enzymatic activity that catalyzes the transamination between _{L}-cysteine and α-ketoglutarate to produce 3-mercaptopyruvate and glutamate. Many distinct cysteine aminotransferase enzymes are also known in the art (see, e.g., Kabil et al. (2014) Biochim. Biophys. Acta 1844(8): 1355-1366).

In one embodiment, the cysteine transaminase gene is a CAT gene. In one embodiment, the cysteine transaminase gene is a E. coli CAT gene.

In another embodiment, the cysteine catabolism enzyme is a cystathionine β-lyase (EC 4.4.1.8). Many distinct cysteine aminotransferase enzymes are also known in the art (see, e.g., Rossol and Paler (1992) J. Bacteriol. 174(9): 2968-77; and Dwivedi et al. (1982) Biochemistry 21(13): 3064-9).

In one embodiment, the cystathionine β-lyase gene is a metC gene. In one embodiment, the cystathionine β-lyase gene is a Escherichia coli metC gene.

In one embodiment, the metC gene has at least about 80% identity with the sequence of SEQ ID NO:78. Accordingly, in one embodiment, the metC gene has at least about 90% identity with the sequence of SEQ ID NO:78. Accordingly, in one embodiment, the metC gene has at least about 95% identity with the sequence of SEQ ID NO:78. Accordingly, in one embodiment, the metC gene has at least about 85%, 86%, 87%, 88%, 89%, 90%, 91%, 92%, 93%, 94%, 95%, 96%, 97%, 98%, or 99% identity with the sequence of SEQ ID NO:78. In another embodiment, the metC gene comprises the sequence of SEQ ID NO:78. In yet another embodiment the metC gene consists of the sequence of SEQ ID NO:78.

In one embodiment, the cystathionine β-lyase gene is a aecD gene. In one embodiment, the cystathionine β-lyase gene is a Corynebacterium glutamicum aecD gene.

In another embodiment, the cysteine catabolism enzyme is a cysteine desulfarase (EC 2.8.1.7). As used herein, “cysteine desulfarase” refers to any polypeptide having enzymatic activity that catalyzes the conversion of cysteine to produce alanine. Many distinct cysteine desulfarase enzymes are also known in the art (see, e.g., Mihara and Esaki (2002) Appl. Microbiol. Biotechnol. 60(1-2): 12-23).

In one embodiment, the cysteine desulfarase gene is an iscS gene. In one embodiment, the cysteine desulfarase gene is a Escherichia coli iscS gene. In one embodiment, the cysteine desulfarase gene is a Helicobacter pylori cysteine desulfarase gene.

In one embodiment, the cysteine desulfarase gene has at least about 80% identity with the sequence of SEQ ID NO:81. Accordingly, in one embodiment, the cysteine desulfarase gene has at least about 90% identity with the sequence of SEQ ID NO:81. Accordingly, in one embodiment, the cysteine desulfarase gene has at least about 95% identity with the sequence of SEQ ID NO:81. Accordingly, in one embodiment, the cysteine desulfarase gene has at least about 85%, 86%, 87%, 88%, 89%, 90%, 91%, 92%, 93%, 94%, 95%, 96%, 97%, 98%, or 99% identity with the sequence of SEQ ID NO:81. In another embodiment, the cysteine desulfarase gene comprises the sequence of SEQ ID NO:81. In yet another embodiment the cysteine desulfarase gene consists of the sequence of SEQ ID NO:81.

The present disclosure further comprises genes encoding functional fragments of a cysteine amino acid catabolism enzyme or functional variants of a cysteine amino acid catabolism enzyme.

Assays for testing the activity of a cysteine catabolism enzyme, a cysteine catabolism enzyme functional variant, or a cysteine catabolism enzyme functional fragment are well known to one of ordinary skill in the art. For example, cysteine catabolism can be assessed by expressing the protein, functional variant, or fragment thereof, in a recombinant bacterial cell that lacks endogenous cysteine catabolism enzyme activity.

In another embodiment, the gene encoding the cysteine catabolism enzyme is co-expressed with an additional cysteine catabolism enzyme. For example, a cysteine desulfhydrase enzyme is co-expressed with a cysteine aminotransferase enzyme.

In one embodiment, the bacterial cell comprises a heterologous gene encoding a cysteine catabolism enzyme. In one embodiment, the bacterial cell comprises a heterologous gene encoding a transporter of cysteine and a heterologous gene encoding a cysteine catabolism enzyme. In one embodiment, the bacterial cell comprises a heterologous gene encoding a cysteine catabolism enzyme and a genetic modification that reduces export of cysteine. In one embodiment, the bacterial cell comprises a heterologous gene encoding a transporter of cysteine, a heterologous gene encoding a cysteine catabolism enzyme, and a genetic modification that reduces export of cysteine. Transporters and exporters are described in more detail in the subsections, below.

B. Transporters of Cysteine

Cysteine transporters may be expressed or modified in the recombinant bacteria described herein in order to enhance cysteine transport into the cell. Specifically, when the transporter of cysteine is expressed in the recombinant bacterial cells described herein, the bacterial cells import more cysteine into the cell when the transporter is expressed than unmodified bacteria of the same bacterial subtype under the same conditions. Thus, the genetically engineered bacteria comprising a heterologous gene encoding transporter of cysteine which may be used to import cysteine into the bacteria so that any gene encoding a cysteine catabolism enzyme expressed in the organism can catabolize the cysteine to treat a disease associated with cysteine, such as cancer.

The uptake of cysteine into bacterial cells is mediated by proteins well known to those of skill in the art.

In some embodiments, the transporter of cysteine is encoded by a transporter of cysteine gene derived from a bacterial genus or species, including but not limited to, Escherichia coli. In some embodiments, the bacterial species is Escherichia coli. In some embodiments, the bacterial species is Escherichia coli strain Nissle.

Assays for testing the activity of a transporter of cysteine, a functional variant of a transporter of cysteine, or a functional fragment of transporter of cysteine are well known to one of ordinary skill in the art.

In one embodiment, when the transporter of a cysteine is expressed in the recombinant bacterial cells described herein, the bacterial cells import 10% more cysteine into the bacterial cell when the transporter is expressed than unmodified bacteria of the same bacterial subtype under the same conditions. In another embodiment, when the transporter of cysteine is expressed in the recombinant bacterial cells described herein, the bacterial cells import 20%, 30%, 40%, 50%, 60%, 70%, 80%, 90% or 100% more cysteine into the bacterial cell when the transporter is expressed than unmodified bacteria of the same bacterial subtype under the same conditions. In yet another embodiment, when the transporter of cysteine is expressed in the recombinant bacterial cells described herein, the bacterial cells import two-fold more cysteine into the cell when the transporter is expressed than unmodified bacteria of the same bacterial subtype under the same conditions. In yet another embodiment, when the transporter of cysteine is expressed in the recombinant bacterial cells described herein, the bacterial cells import three-fold, four-fold, five-fold, six-fold, seven-fold, eight-fold, nine-fold, or ten-fold more cysteine into the cell when the transporter is expressed than unmodified bacteria of the same bacterial subtype under the same conditions.

C. Exporters of Cysteine

Cysteine exporters may be modified in the recombinant bacteria described herein in order to reduce cysteine export from the cell. Specifically, when the recombinant bacterial cells described herein comprise a genetic modification that reduces export of cysteine, the bacterial cells retain more cysteine in the bacterial cell than unmodified bacteria of the same bacterial subtype under the same conditions. Thus, the recombinant bacteria comprising a genetic modification that reduces export of cysteine may be used to retain more cysteine in the bacterial cell so that any cysteine catabolism enzyme expressed in the organism, e.g., a co-expressed cysteine catabolism enzyme, can catabolize the cysteine.

In one embodiment, the genetic modification is a mutation in an endogenous gene encoding an exporter of cysteine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export cysteine from the bacterial cell.

Multiple cysteine exporters are known in the art. For example, the cysteine exporters YdeD, YfiK and Bcr mediate the export of cysteine from the cytoplasm of Escherichia coli into the periplasm (Ohtsu et al., J. Biol. Chem. 285: 117479-87). It has been suggested that TolC further mediates the export of cysteine from the periplasm to the cell exterior. Additional cysteine exporters have been identified and are disclosed known in the art.

In one embodiment, the cysteine exporter gene is a ydeD gene. In another embodiment, the cysteine exporter gene is a Escherichia coli ydeD gene. In another embodiment, the cysteine exporter gene is a Bacillus atrophaeusi ydeD gene.

In one embodiment, the ydeD gene has at least about 80% identity with the sequence of SEQ ID NO:82. Accordingly, in one embodiment, the ydeD gene has at least about 90% identity with the sequence of SEQ ID NO:82. Accordingly, in one embodiment, the ydeD gene has at least about 95% identity with the sequence of SEQ ID NO:82. Accordingly, in one embodiment, the ydeD gene has at least about 85%, 86%, 87%, 88%, 89%, 90%, 91%, 92%, 93%, 94%, 95%, 96%, 97%, 98%, or 99% identity with the sequence of SEQ ID NO:82. In another embodiment, the ydeD gene comprises the sequence of SEQ ID NO:82. In yet another embodiment the ydeD gene consists of the sequence of SEQ ID NO:82.

In one embodiment, the cysteine exporter gene is a yfiK gene. In another embodiment, the cysteine exporter gene is a Escherichia coli yfiK gene.

In one embodiment, the yfiK gene has at least about 80% identity with the sequence of SEQ ID NO:83. Accordingly, in one embodiment, the yfiK gene has at least about 90% identity with the sequence of SEQ ID NO:83. Accordingly, in one embodiment, the yfiK gene has at least about 95% identity with the sequence of SEQ ID NO:83. Accordingly, in one embodiment, the yfiK gene has at least about 85%, 86%, 87%, 88%, 89%, 90%, 91%, 92%, 93%, 94%, 95%, 96%, 97%, 98%, or 99% identity with the sequence of SEQ ID NO:83. In another embodiment, the yfiK gene comprises the sequence of SEQ ID NO:83. In yet another embodiment the yfiK gene consists of the sequence of SEQ ID NO:83.

In one embodiment, the cysteine exporter gene is a bcr gene. In another embodiment, the cysteine exporter gene is a Escherichia coli bcr gene.

In one embodiment, the bcr gene has at least about 80% identity with the sequence of SEQ ID NO:84. Accordingly, in one embodiment, the bcr gene has at least about 90% identity with the sequence of SEQ ID NO:84. Accordingly, in one embodiment, the bcr gene has at least about 95% identity with the sequence of SEQ ID NO:84. Accordingly, in one embodiment, the bcr gene has at least about 85%, 86%, 87%, 88%, 89%, 90%, 91%, 92%, 93%, 94%, 95%, 96%, 97%, 98%, or 99% identity with the sequence of SEQ ID NO:84. In another embodiment, the bcr gene comprises the sequence of SEQ ID NO:84. In yet another embodiment the bcr gene consists of the sequence of SEQ ID NO:84.

In one embodiment, the cysteine exporter gene is a tolC gene. In another embodiment, the cysteine exporter gene is a Escherichia coli tolC gene.

In one embodiment, the tolC gene has at least about 80% identity with the sequence of SEQ ID NO:85. Accordingly, in one embodiment, the tolC gene has at least about 90% identity with the sequence of SEQ ID NO:85. Accordingly, in one embodiment, the tolC gene has at least about 95% identity with the sequence of SEQ ID NO:85. Accordingly, in one embodiment, the tolC gene has at least about 85%, 86%, 87%, 88%, 89%, 90%, 91%, 92%, 93%, 94%, 95%, 96%, 97%, 98%, or 99% identity with the sequence of SEQ ID NO:85. In another embodiment, the tolC gene comprises the sequence of SEQ ID NO:85. In yet another embodiment the tolC gene consists of the sequence of SEQ ID NO:85.

Assays for testing the activity of an exporter of a cysteine are well known to one of ordinary skill in the art (Yamada et al. (2006) Appl. Environ. Microbiol. 72(7): 4735-4742).

In another embodiment, the genetic modification is a mutation in a promoter of an endogenous gene encoding an exporter of cysteine.

In yet another embodiment, the genetic modification is an overexpression of a repressor of an exporter of cysteine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

11. Tyrosine

A. Tyrosine Catabolism Enzymes

Tyrosine catabolism enzymes may be expressed or modified in the bacteria disclosed herein in order to enhance catabolism of tyrosine. As used herein, the term “tyrosine catabolism enzyme” refers to an enzyme involved in the catabolism of tyrosine. Specifically, when a tyrosine catabolism enzyme is expressed in a recombinant bacterial cell, the bacterial cell hydrolyzes more tyrosine into glutamate when the catabolism enzyme is expressed than unmodified bacteria of the same bacterial subtype under the same conditions. Thus, the genetically engineered bacteria comprising a heterologous gene encoding a tyrosine catabolism enzyme can catabolize tyrosine to treat a disease associated with tyrosine, such as cancer.

In one embodiment, the tyrosine catabolism enzyme increases the rate of tyrosine catabolism in the cell. In one embodiment, the tyrosine catabolism enzyme decreases the level of tyrosine in the cell. In another embodiment, the tyrosine catabolism enzyme increases the level of glutamate in the cell.

Tyrosine catabolism enzymes are well known to those of skill in the art (see, e.g., Aklujkar et al., Microbiology, 160:2694-2709, 2014). In bacteria such as Ferroglobus placidus, tyrosine catabolism enzymes are capable of converting tyrosine to 4-hydroxypenylpyruvate and glutamate, and subsequently decarboxylate 4-hydroxyphenylpyruvate into hydroxyphenylacetaldehyde, which is then oxidized into 4-hydroxyphenylacetate by one of several aldehyde:ferredoxin oxioreductases (Aklujkar et al. 2014).

In some embodiments, a tyrosine catabolism enzyme is encoded by a gene encoding a tyrosine catabolism enzyme derived from a bacterial species. In some embodiments, a tyrosine catabolism enzyme is encoded by a gene encoding a tyrosine catabolism enzyme derived from a non-bacterial species. In some embodiments, a tyrosine catabolism enzyme is encoded by a gene derived from a eukaryotic species, e.g., a yeast species or a plant species. In one embodiment, the gene encoding the tyrosine catabolism enzyme is derived from an organism of the genus or species that includes, but is not limited to, Ferroglobus placidus and Sinorhizobium meliloti.

In one embodiment, the tyrosine catabolism enzyme is tyrosine transaminase. For example, the tyrosine transaminase enzyme of Sinorhizobium meliloti is capable of metabolizing tyrosine (see, e.g., Aklujkar et al. 2014). In one embodiment, the tyrosine transaminase gene is a is a Sinorhizobium meliloti tyrosine transaminase gene.

In one embodiment, the tyrosine transaminase gene has at least about 80% identity with the sequence of SEQ ID NO:86. Accordingly, in one embodiment, the tyrosine transaminase gene has at least about 90% identity with the sequence of SEQ ID NO:86. Accordingly, in one embodiment, the tyrosine transaminase gene has at least about 95% identity with the sequence of SEQ ID NO:86. Accordingly, in one embodiment, the tyrosine transaminase gene has at least about 85%, 86%, 87%, 88%, 89%, 90%, 91%, 92%, 93%, 94%, 95%, 96%, 97%, 98%, or 99% identity with the sequence of SEQ ID NO:86. In another embodiment, the tyrosine transaminase gene comprises the sequence of SEQ ID NO:86. In yet another embodiment the tyrosine transaminase gene consists of the sequence of SEQ ID NO:86.

The present disclosure further comprises genes encoding functional fragments of a tyrosine amino acid catabolism enzyme or functional variants of a tyrosine amino acid catabolism enzyme.

Assays for testing the activity of a tyrosine catabolism enzyme, a tyrosine catabolism enzyme functional variant, or a tyrosine catabolism enzyme functional fragment are well known to one of ordinary skill in the art. For example, tyrosine catabolism can be assessed by expressing the protein, functional variant, or fragment thereof, in a recombinant bacterial cell that lacks endogenous tyrosine catabolism enzyme activity. Tyrosine catabolism can be assessed using methods well known to one of ordinary skill in the art (see, e.g., Aklujkar et al. 2014), the entire contents of which are incorporated by reference).

In one embodiment, the bacterial cell comprises a heterologous gene encoding a tyrosine catabolism enzyme. In one embodiment, the bacterial cell comprises a heterologous gene encoding a transporter of tyrosine and a heterologous gene encoding a tyrosine catabolism enzyme. In one embodiment, the bacterial cell comprises a heterologous gene encoding a tyrosine catabolism enzyme and a genetic modification that reduces export of tyrosine. In one embodiment, the bacterial cell comprises a heterologous gene encoding a transporter of tyrosine, a heterologous gene encoding a tyrosine catabolism enzyme, and a genetic modification that reduces export of tyrosine. Transporters and exporters are described in more detail in the subsections, below.

B. Transporters of Tyrosine

Tyrosine transporters may be expressed or modified in the recombinant bacteria described herein in order to enhance tyrosine transport into the cell. Specifically, when the transporter of tyrosine is expressed in the recombinant bacterial cells described herein, the bacterial cells import more tyrosine into the cell when the transporter is expressed than unmodified bacteria of the same bacterial subtype under the same conditions. Thus, the genetically engineered bacteria comprising a heterologous gene encoding transporter of tyrosine which may be used to import tyrosine into the bacteria so that any gene encoding a tyrosine catabolism enzyme expressed in the organism can catabolize the tyrosine to treat a disease associated with tyrosine, such as cancer.

The uptake of tyrosine into bacterial cells is mediated by proteins well known to those of skill in the art. For example, a tyrosine transporter TyrP has been identified in Lactobacillus brevis (Wolken et al., J. Bacteriol., 188(6): 2198-2206, 2006) and Escherichia coli.

In one embodiment, the at least one gene encoding a transporter of tyrosine is the tyrP gene. In one embodiment, the bacterial cell described herein has been genetically engineered to comprise at least one heterologous tyrP gene. In one embodiment, the at least one gene encoding a transporter of tyrosine is the Escherichia coli tyrP gene. In one embodiment, the at least one gene encoding a transporter of tyrosine is the Lactobacillus brevi tyrP gene.

In one embodiment, the tyrP gene has at least about 80% identity with the sequence of SEQ ID NO:87. Accordingly, in one embodiment, the tyrP gene has at least about 90% identity with the sequence of SEQ ID NO:87. Accordingly, in one embodiment, the tyrP gene has at least about 95% identity with the sequence of SEQ ID NO:87. Accordingly, in one embodiment, the tyrP gene has at least about 85%, 86%, 87%, 88%, 89%, 90%, 91%, 92%, 93%, 94%, 95%, 96%, 97%, 98%, or 99% identity with the sequence of SEQ ID NO:87. In another embodiment, the tyrP gene comprises the sequence of SEQ ID NO:87. In yet another embodiment the tyrP gene consists of the sequence of SEQ ID NO:87.

In some embodiments, the transporter of tyrosine is encoded by a transporter of tyrosine gene derived from a bacterial genus or species, including but not limited to, Escherichia coli or Lactobacillus brevis. In some embodiments, the bacterial species is Escherichia coli. In some embodiments, the bacterial species is Escherichia coli strain Nissle.

Assays for testing the activity of a transporter of tyrosine, a functional variant of a transporter of tyrosine, or a functional fragment of transporter of tyrosine are well known to one of ordinary skill in the art. For example, import of tyrosine may be determined using the methods as described in Wolken et al., J. Bacteriol., 188(6):2198-2206, 2006, the entire contents of which are expressly incorporated by reference herein.

In one embodiment, when the transporter of a tyrosine is expressed in the recombinant bacterial cells described herein, the bacterial cells import 10% more tyrosine into the bacterial cell when the transporter is expressed than unmodified bacteria of the same bacterial subtype under the same conditions. In another embodiment, when the transporter of tyrosine is expressed in the recombinant bacterial cells described herein, the bacterial cells import 20%, 30%, 40%, 50%, 60%, 70%, 80%, 90% or 100% more tyrosine into the bacterial cell when the transporter is expressed than unmodified bacteria of the same bacterial subtype under the same conditions. In yet another embodiment, when the transporter of tyrosine is expressed in the recombinant bacterial cells described herein, the bacterial cells import two-fold more tyrosine into the cell when the transporter is expressed than unmodified bacteria of the same bacterial subtype under the same conditions. In yet another embodiment, when the transporter of tyrosine is expressed in the recombinant bacterial cells described herein, the bacterial cells import three-fold, four-fold, five-fold, six-fold, seven-fold, eight-fold, nine-fold, or ten-fold more tyrosine into the cell when the transporter is expressed than unmodified bacteria of the same bacterial subtype under the same conditions.

C. Exporters of Tyrosine

Tyrosine exporters may be modified in the recombinant bacteria described herein in order to reduce tyrosine export from the cell. Specifically, when the recombinant bacterial cells described herein comprise a genetic modification that reduces export of tyrosine, the bacterial cells retain more tyrosine in the bacterial cell than unmodified bacteria of the same bacterial subtype under the same conditions. Thus, the recombinant bacteria comprising a genetic modification that reduces export of tyrosine may be used to retain more tyrosine in the bacterial cell so that any tyrosine catabolism enzyme expressed in the organism, e.g., co-expressed tyrosine catabolism enzyme, can catabolize the tyrosine.

In one embodiment, the genetic modification is a mutation in an endogenous gene encoding an exporter of tyrosine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export tyrosine from the bacterial cell.

In another embodiment, the genetic modification is a mutation in a promoter of an endogenous gene encoding an exporter of tyrosine.

In yet another embodiment, the genetic modification is an overexpression of a repressor of an exporter of tyrosine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

12. Phenylalanine

A. Phenylalanine Catabolism Enzymes

Phenylalanine catabolism enzymes may be expressed or modified in the bacteria disclosed herein in order to enhance catabolism of phenylalanine. As used herein, the term “phenylalanine catabolism enzyme” refers to an enzyme involved in the catabolism of phenylalanine. Specifically, when a phenylalanine catabolism enzyme is expressed in a recombinant bacterial cell, the bacterial cell converts more phenylalanine into trans-cinnamic acid, ammonia, and/or tyrosine when the catabolism enzyme is expressed than unmodified bacteria of the same bacterial subtype under the same conditions. Thus, the genetically engineered bacteria comprising a heterologous gene encoding a phenylalanine catabolism enzyme can catabolize phenylalanine to treat a disease associated with phenylalanine, such as cancer, e.g., melanoma and breast cancer. See PCT/US2016/32562, filed May 13, 2016 and PCT/US2016/062369, filed Nov. 16, 2016, both of which applications are hereby incorporated by reference in their entireties, including he drawings.

In one embodiment, the phenylalanine catabolism enzyme increases the rate of phenylalanine catabolism in the cell. In one embodiment, the phenylalanine catabolism enzyme decreases the level of phenylalanine in the cell. In another embodiment, the phenylalanine catabolism enzyme increases the level of trans-cinammic acid in the cell. In another embodiment, phenylalanine catabolism enzyme increases the level of ammonia in the cell. In another embodiment, phenylalanine catabolism enzyme increases the level of tyrosine in the cell.

Phenylalanine catabolism enzymes are well known to those of skill in the art (see, e.g., Sarkissian et al. (1999) Proc. Natl. Acad. Sci. USA 96(5): 2339-44; Xiang et al. (2005) J. Bacteriol. 187(12): 4286-9; Kobe et al. (1997) Protein Sci. 6(6): 1352-7; Kwok et al. (1985) Biochemistry 24(3): 556-61). For example, phenylalanine ammonia lyase (PAL; E.C. 4.3.1.24) is capable of converting phenylalanine into ammonia and trans-cinnamic acid. Phenylalanine is also converted into tyrosine by phenylalanine hydroxylase (PAH; E.C. 1.14.16.1). In other embodiments, phenylalanine is converted to phenylpyruvate by amino acid oxidase (also known as amino acid deaminase) (L-AAD gene).

In some embodiments, a phenylalanine catabolism enzyme is encoded by a gene encoding a phenylalanine catabolism enzyme derived from a bacterial species. In some embodiments, a phenylalanine catabolism enzyme is encoded by a gene encoding a phenylalanine catabolism enzyme derived from a non-bacterial species. In some embodiments, a phenylalanine catabolism enzyme is encoded by a gene derived from a eukaryotic species, e.g., a yeast species or a plant species. In one embodiment, the gene encoding the phenylalanine catabolism enzyme is derived from an organism of the genus or species that includes, but is not limited to, Achromobacter, Agrobacterium, Anabaena, Arabidopsis, Colwellia, Photorhabdus, Legionella, Pseudomonas, Streptomyces, Rhodosporidium, Rhodotorula, Achromobacter xylosoxidans, Agrobacterium tumefaciens, Anabaena variabilis, Arabidopsis thaliana, Colwellia psychrerythraea, Homo sapiens, Legionella pneumophila, Photorhabdus luminescens, Pseudomonas aeruginosa, Streptomyces verticillatus, Rhodosporidium toruloides, Rhodotorula glutinis, Proteus vulgaris and Proteus mirabilis.

In one embodiment, the phenylalanine catabolism enzyme is a phenylalanine ammonia lyase (“PAL”). As used herein, “phenylalanine ammonia lyase” refers to any polypeptide having enzymatic activity that catalyzes the conversion of phenylalanine to ammonia and trans-cinnamic acid. For example, the PAL enzyme of the yeast Rhodotorula glutinis is capable of metabolizing phenylalanine (see, e.g., Hodgins (1971) J. Biol. Chem. 246: 2977-85). Other distinct PAL enzymes are also known in the art (see, e.g., Gilbert et. al. (1985) J. Bacteriol. 161: 314-20; Sarkissian et al. (1999) Proc. Natl. Acad. Sci. USA 96(5): 2339-44; Xiang et al. (2005) J. Bacteriol. 187(12): 4286-9).

In one embodiment, the PAL enzyme is encoded by a PAL gene. In another embodiment, the PAL enzyme is encoded by a PAL1 gene. In one embodiment, the PAL1 gene is the Anabaena variabilis PAL1 gene. In one embodiment, the PAL enzyme is encoded by a PAL3 gene. In one embodiment, the PAL3 gene is the Photorhabdus luminescens PAL3 gene.

In one embodiment, the PAL1 gene has at least about 80% identity with the sequence of SEQ ID NO:99. Accordingly, in one embodiment, the PAL1 gene has at least about 90% identity with the sequence of SEQ ID NO:99. Accordingly, in one embodiment, the PAL1 gene has at least about 95% identity with the sequence of SEQ ID NO:99. Accordingly, in one embodiment, the PAL1 gene has at least about 85%, 86%, 87%, 88%, 89%, 90%, 91%, 92%, 93%, 94%, 95%, 96%, 97%, 98%, or 99% identity with the sequence of SEQ ID NO:99. In another embodiment, the PAL1 gene comprises the sequence of SEQ ID NO:99. In yet another embodiment the PAL1 gene consists of the sequence of SEQ ID NO:99.

In one embodiment, the PAL3 gene has at least about 80% identity with the sequence of SEQ ID NO:100. Accordingly, in one embodiment, the PAL3 gene has at least about 90% identity with the sequence of SEQ ID NO:100. Accordingly, in one embodiment, the PAL3 gene has at least about 95% identity with the sequence of SEQ ID NO:100. Accordingly, in one embodiment, the PAL3 gene has at least about 85%, 86%, 87%, 88%, 89%, 90%, 91%, 92%, 93%, 94%, 95%, 96%, 97%, 98%, or 99% identity with the sequence of SEQ ID NO:100. In another embodiment, the PAL3 gene comprises the sequence of SEQ ID NO:100. In yet another embodiment the PAL3 gene consists of the sequence of SEQ ID NO:100.

In another embodiment, the phenylalanine catabolism enzyme is a phenylalanine hydroxylase (“PAH”). As used herein, “phenylalanine hydroxylase” refers to any polypeptide having enzymatic activity that catalyzes the hydroxylation of phenylalanine to generate tyrosine. In one embodiment, the PAH enzyme requires the co-factor tetrahydrobiopterin. For example, the phenylalanine hydroxylase enzyme of Legionella pneumophila (encoded by the phhA gene) is capable of metabolizing phenylalanine (see, e.g., Flydal et al. (2012) PLoS One 7, e46209). Other distinct phenylalanine dehydrogenase enzymes are also known in the art (see, e.g., Flydal and Martinez (2013) IUBMB Life 65(4): 341-9).

In one embodiment, the phenylalanine hydroxylase gene is a phhA gene. In another embodiment, the phhA gene is a Legionella pneumophila phhA gene. In one embodiment, the phhA gene is a Colwellia psychrerythraea phhA gene. In another embodiment, the phhA gene is a Pseudomonas aeruginosa phhA gene. In one embodiment, the phhA gene is a Chromobacterium violaceum phhA gene. In another embodiment, the phenylalanine hydroxylase gene is a PAH gene. In one embodiment, the PAH gene is a Homo sapiens PAH gene.

In one embodiment, the phhA gene has at least about 80% identity with the sequence of SEQ ID NO:101. Accordingly, in one embodiment, the phhA gene has at least about 90% identity with the sequence of SEQ ID NO:101. Accordingly, in one embodiment, the phhA gene has at least about 95% identity with the sequence of SEQ ID NO:101. Accordingly, in one embodiment, the phhA gene has at least about 85%, 86%, 87%, 88%, 89%, 90%, 91%, 92%, 93%, 94%, 95%, 96%, 97%, 98%, or 99% identity with the sequence of SEQ ID NO:101. In another embodiment, the phhA gene comprises the sequence of SEQ ID NO:101. In yet another embodiment the phhA gene consists of the sequence of SEQ ID NO:101.

In one embodiment, the phenylalanine catabolism enzyme is amino acid oxidase (also known as amino acid deaminase) (“L-AAD”). As used herein, “amino acid oxidase” or “amino acid deaminase” refers to any polypeptide having enzymatic activity that catalyzes the conversion of phenylalanine to phenylpyruvate. For example, the L-AAD enzyme of the yeast Proteus mirabilis is capable of metabolizing phenylalanine (see, e.g., (Hou et al. 2015, Appl Microbiol Biotechnol. 2015 October; 99(20):8391-402; “Production of phenylpyruvic acid from L-phenylalanine using an L-amino acid deaminase from Proteus mirabilis: comparison of enzymatic and whole-cell biotransformation approaches”). Other L-AAD enzymes are also known in the art (see, e.g., Song et al., Scientific Reports, Nature, 5:12694; DOI: 10:1038/srep12694 (2015)). Proteus mirabilis contains two types of L-AADs (Duerre and Chakrabarty 1975). One has broad substrate specificity and catalyzes the oxidation of aliphatic and aromatic L-amino acids to keto acids, typically L-phenylalanine (GenBank: U35383.1) (Baek et al., Journal of Basic Microbiology 2011, 51, 129-135; “Expression and characterization of a second L-amino acid deaminase isolated from Proteus mirabilis in Escherichia coli”). The other type acts mainly on basic L-amino acids (GenBank: EU669819.1). Most eukaryotic and prokaryotic L-amino acid deaminases are extracellularly secreted, with the exception of from Proteus species LAADs, which are membrane-bound. In Proteus mirabilis, L-AADs have been reported to be located in the plasma membrane, facing outward into the periplasmic space, in which the enzymatic activity resides (Pelmont J et al., (1972) “L-amino acid oxidases of Proteus mirabilis: general properties” Biochimie 54: 1359-1374).The present disclosure further comprises genes encoding functional fragments of a phenylalanine amino acid catabolism enzyme or functional variants of a phenylalanine amino acid catabolism enzyme.

In some embodiments, the disclosure provides genetically engineered bacteria that encode and express a phenylalanine metabolizing enzyme (PME). In some embodiments, the disclosure provides genetically engineered bacteria that encode and express phenylalanine ammonia lyase and/or phenylalanine hydroxylase and/or L-aminoacid deaminase and are capable of reducing hyperphenylalaninemia.

The enzyme phenylalanine ammonia lyase (PAL) is capable of metabolizing phenylalanine to non-toxic levels of ammonia and transcinnamic acid. Unlike PAH, PAL does not require THB cofactor activity in order to metabolize phenylalanine. L-amino acid deaminase (LAAD) catalyzes oxidative deamination of phenylalanine to generate phenylpyruvate, and trace amounts of ammonia and hydrogen peroxide. Phenylpyruvic acid (PPA) is widely used in the pharmaceutical, food, and chemical industries, and PPA is the starting material for the synthesis of D-phenylalanine, a raw intermediate in the production of many chiral drugs and food additives. LAAD has therefore been studied in the context of industrial PPA production (Hou et al. 2015, Appl Microbiol Biotechnol. 2015 October; 99(20):8391-402; “Production of phenylpyruvic acid from L-phenylalanine using an L-amino acid deaminase from Proteus mirabilis: comparison of enzymatic and whole-cell biotransformation approaches”). Phenylpyruvate is unable to cross the blood brain barrier (Steele, Fed Proc. 1986 June; 45(7):2060-4; “Blood-brain barrier transport of the alpha-keto acid analogs of amino acids.,” indicating that this conversion is useful in controlling the neurological phenotypes of PKU.

In some embodiments, the disclosure provides genetically engineered bacteria that encode and express a phenylalanine metabolizing enzyme (PME). In some embodiments, the disclosure provides genetically engineered bacteria that encode and express phenylalanine ammonia lyase (PAL) and/or phenylalanine hydroxylase (PAH) and/or L-aminoacid deaminase (L-AAD) and are capable of reducing hyperphenylalaninemia.

In some embodiments, the genetically engineered bacteria comprise a gene sequence encoding one or more non-native phenylalanine ammonia lyase (PAL). In some embodiments, the genetically engineered bacteria comprise a gene sequence encoding one or more non-native phenylalanine hydroxylase (PAH). In some embodiments, the genetically engineered bacteria comprise a gene sequence encoding one or more non-native L-aminoacid deaminase (L-AAD). In some embodiments, the genetically engineered bacteria comprise a gene sequence encoding one or more phenylalanine transporter, e.g., PheP. In some embodiments, the genetically engineered bacteria comprise a gene sequence encoding one or more non-native phenylalanine ammonia lyase (PAL) and are capable of processing and reducing phenylalanine in a mammal. In some embodiments, the genetically engineered bacteria comprise a gene sequence encoding one or more non-native phenylalanine hydroxylase (PAH) and are capable of processing and reducing phenylalanine in a mammal. In some embodiments, the genetically engineered bacteria comprise a gene sequence encoding one or more non-native L-aminoacid deaminase (L-AAD) and are capable of processing and reducing phenylalanine in a mammal. In some embodiments, the genetically engineered bacteria comprise a gene sequence encoding one or more non-native phenylalanine ammonia lyase (PAL) and gene sequence encoding one or more non-native L-aminoacid deaminase (L-AAD). In some embodiments, the genetically engineered bacteria comprise a gene sequence encoding one or more non-native phenylalanine ammonia lyase (PAL) and gene sequence encoding one or more phenylalanine transporter, e.g., PheP. In some embodiments, the genetically engineered bacteria comprise a gene sequence encoding one or more non-native L-aminoacid deaminase (L-AAD) and gene sequence encoding one or more phenylalanine transporter, e.g., PheP. In some embodiments, the genetically engineered bacteria comprise a gene sequence encoding one or more non-native phenylalanine ammonia lyase (PAL), gene sequence encoding one or more non-native L-aminoacid deaminase (L-AAD), and gene sequence encoding one or more phenylalanine transporter, e.g., PheP.

The engineered bacteria may also contain one or more gene sequences relating to bio-safety and/or bio-containment, e.g., a kill-switch, gene guard system, and/or auxotrophy. In some embodiments, the engineered bacteria may contain an antibiotic resistance gene. The expression of any these gene sequence(s) may be regulated using a variety of promoter systems, such as any of the promoter systems disclosed herein, which promoter system may involve use of the same promoter to regulate one or more different genes, may involve use of a different copy of the same promoter to regulate different genes, and/or may involve the use of different promoters used in combination to regulate the expression of different genes. The use of different regulatory or promoter systems to control gene expression provides flexibility (e.g., the ability to differentially control gene expression under different environmental conditions and/or the ability to differentially control gene expression temporally) and also provides the ability to “fine-tune” gene expression, any or all of which regulation may serve to optimize gene expression and/or growth of the bacteria.

In certain embodiments, the genetically engineered bacteria are non-pathogenic and may be introduced into the gut in order to reduce toxic levels of phenylalanine. In certain embodiments, the phenylalanine ammonia lyase and/or phenylalanine hydroxylase and/or L-aminoacid deaminase is stably produced by the genetically engineered bacteria, and/or the genetically engineered bacteria are stably maintained in vivo and/or in vitro. In certain embodiments, the genetically engineered bacteria further comprise a phenylalanine transporter gene to increase their uptake of phenylalanine. The invention also provides pharmaceutical compositions comprising the genetically engineered bacteria, and methods of modulating and treating disorders associated with hyperphenylalaninemia.

Assays for testing the activity of a phenylalanine catabolism enzyme, a phenylalanine catabolism enzyme functional variant, or a phenylalanine catabolism enzyme functional fragment are well known to one of ordinary skill in the art. For example, phenylalanine catabolism can be assessed by expressing the protein, functional variant, or fragment thereof, in a recombinant bacterial cell that lacks endogenous phenylalanine catabolism enzyme activity. Phenylalanine ammonia lyase activity can be assessed as described in Gilbert and Jack (1981) Biochem. J. 199: 715-723, the entire contents of which are incorporated by reference. Phenylalanine hydroxylase activity can be assessed by measuring the conversion of phenylalanine to tyrosine (see, e.g., Flydal et al. (2012) PLoS One 7, e46209, the entire contents of which are incorporated by reference).

In another embodiment, the gene encoding the phenylalanine catabolism enzyme is co-expressed with an additional phenylalanine catabolism enzyme, for example, a phenylalanine ammonia lyase enzyme is co-expressed with a phenylalanine hydroxylase enzyme.

In one embodiment, the bacterial cell comprises a heterologous gene encoding a phenylalanine catabolism enzyme. In one embodiment, the bacterial cell comprises a heterologous gene encoding a transporter of phenylalanine and a heterologous gene encoding a phenylalanine catabolism enzyme. In one embodiment, the bacterial cell comprises a heterologous gene encoding a phenylalanine catabolism enzyme and a genetic modification that reduces export of phenylalanine. In one embodiment, the bacterial cell comprises a heterologous gene encoding a transporter of phenylalanine, a heterologous gene encoding a phenylalanine catabolism enzyme, and a genetic modification that reduces export of phenylalanine. Transporters and exporters are described in more detail in the subsections, below.

B. Transporters of Phenylalanine

Phenylalanine transporters may be expressed or modified in the recombinant bacteria described herein in order to enhance phenylalanine transport into the cell. Specifically, when the transporter of phenylalanine is expressed in the recombinant bacterial cells described herein, the bacterial cells import more phenylalanine into the cell when thetransporter is expressed than unmodified bacteria of the same bacterial subtype under the same conditions. Thus, the genetically engineered bacteria comprising a heterologous gene encoding transporter of phenylalanine which may be used to import phenylalanine into the bacteria so that any gene encoding a phenylalanine catabolism enzyme expressed in the organism can catabolize the phenylalanine to treat a disease associated with phenylalanine, such as cancer.

The uptake of phenylalanine into bacterial cells is mediated by proteins well known to those of skill in the art. For example, a phenylalanine transporter PheP has been identified (Pi et al. (1991) J. Bacteriol. 173(12): 3622-9; Pi et al. (1996) J. Bacteriol. 178(9): 2650-5; Pi et al. (1998) J. Bacteriol. 180(21): 5515-9; and Horsburgh et al. (2004) Infect. Immun. 72(5): 3073-3076). Additional phenylalanine transporters have been identified and are known in the art.

In one embodiment, the at least one gene encoding a transporter of phenylalanine is the pheP gene. In one embodiment, the bacterial cell described herein has been genetically engineered to comprise at least one heterologous pheP gene. In one embodiment, the at least one gene encoding a transporter of phenylalanine is the Escherichia coli pheP gene. In one embodiment, the at least one gene encoding a transporter of phenylalanine is the Staphylococcus aureus pheP gene.

In one embodiment, the pheP gene has at least about 80% identity with the sequence of SEQ ID NO:98. Accordingly, in one embodiment, the pheP gene has at least about 90% identity with the sequence of SEQ ID NO:98. Accordingly, in one embodiment, the pheP gene has at least about 95% identity with the sequence of SEQ ID NO:98. Accordingly, in one embodiment, the pheP gene has at least about 85%, 86%, 87%, 88%, 89%, 90%, 91%, 92%, 93%, 94%, 95%, 96%, 97%, 98%, or 99% identity with the sequence of SEQ ID NO:98. In another embodiment, the pheP gene comprises the sequence of SEQ ID NO:98. In yet another embodiment the pheP gene consists of the sequence of SEQ ID NO:98.

In some embodiments, the transporter of phenylalanine is encoded by a transporter of phenylalanine gene derived from a bacterial genus or species, including but not limited to, Escherichia coli or Staphylococcus aureus. In some embodiments, the bacterial species is Escherichia coli. In some embodiments, the bacterial species is Escherichia coli strain Nissle.

Assays for testing the activity of a transporter of phenylalanine, a functional variant of a transporter of phenylalanine, or a functional fragment of transporter of phenylalanine are well known to one of ordinary skill in the art. For example, import of phenylalanine may be determined using the methods as described in Pi et al. (1998) J. Bacteriol. 180(21): 5515-9, the entire contents of which are expressly incorporated by reference herein.

In one embodiment, when the transporter of a phenylalanine is expressed in the recombinant bacterial cells described herein, the bacterial cells import 10% more phenylalanine into the bacterial cell when the transporter is expressed than unmodified bacteria of the same bacterial subtype under the same conditions. In another embodiment, when the transporter of phenylalanine is expressed in the recombinant bacterial cells described herein, the bacterial cells import 20%, 30%, 40%, 50%, 60%, 70%, 80%, 90% or 100% more phenylalanine into the bacterial cell when the transporter is expressed than unmodified bacteria of the same bacterial subtype under the same conditions. In yet another embodiment, when the transporter of phenylalanine is expressed in the recombinant bacterial cells described herein, the bacterial cells import two-fold more phenylalanine into the cell when the transporter is expressed than unmodified bacteria of the same bacterial subtype under the same conditions. In yet another embodiment, when the transporter of phenylalanine is expressed in the recombinant bacterial cells described herein, the bacterial cells import three-fold, four-fold, five-fold, six-fold, seven-fold, eight-fold, nine-fold, or ten-fold more phenylalanine into the cell when the transporter is expressed than unmodified bacteria of the same bacterial subtype under the same conditions.

C. Exporters of Phenylalanine

Phenylalanine exporters may be modified in the recombinant bacteria described herein in order to reduce phenylalanine export from the cell. Specifically, when the recombinant bacterial cells described herein comprise a genetic modification that reduces export of phenylalanine, the bacterial cells retain more phenylalanine in the bacterial cell than unmodified bacteria of the same bacterial subtype under the same conditions. Thus, the recombinant bacteria comprising a genetic modification that reduces export of phenylalanine may be used to retain more phenylalanine in the bacterial cell so that any phenylalanine catabolism enzyme expressed in the organism, e.g., co-expressed phenylalanine catabolism enzyme, can catabolize the phenylalanine.

In one embodiment, the genetic modification is a mutation in an endogenous gene encoding an exporter of phenylalanine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export phenylalanine from the bacterial cell.

Multiple phenylalanine exporters are known in the art. For example, the aromatic amino acid exporter YddG (encoded by the yddG gene) is capable of exporting phenylalanine (Doroshenko et al. (2007) FEMS Microbiol. Lett. 275:312-18). Additional phenylalanine exporters have been identified and are disclosed in the serine section herein. Assays for testing the activity of an exporter of a phenylalanine are well known to one of ordinary skill in the art (Doroshenko et al. (2007) FEMS Microbiol. Lett. 275:312-18).

In another embodiment, the genetic modification is a mutation in a promoter of an endogenous gene encoding an exporter of phenylalanine.

In yet another embodiment, the genetic modification is an overexpression of a repressor of an exporter of phenylalanine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

13. Glutamic Acid

A. Glutamic Acid Catabolism Enzymes

Glutamic acid catabolism enzymes may be expressed or modified in the bacteria disclosed herein in order to enhance catabolism of glutamic acid. As used herein, the term “glutamic acid catabolism enzyme” refers to an enzyme involved in the catabolism of glutamic acid. Specifically, when an glutamic acid catabolism enzyme is expressed in a recombinant bacterial cell, the bacterial cell hydrolyzes more glutamic acid into gamma-aminobutyric acid (γ-Aminobutyric acid) (GABA) when the catabolism enzyme is expressed than unmodified bacteria of the same bacterial subtype under the same conditions. Thus, the genetically engineered bacteria comprising a heterologous gene encoding an glutamic acid catabolism enzyme can catabolize glutamic acid to treat a disease associated with glutamic acid, such as cancer.

In one embodiment, the glutamic acid catabolism enzyme increases the rate of glutamic acid catabolism in the cell. In one embodiment, the glutamic acid catabolism enzyme decreases the level of glutamic acid in the cell. In another embodiment, the glutamic acid catabolism enzyme increases the level of GABA in the cell.

Glutamic acid catabolism enzymes are well known to those of skill in the art (see, e.g., Smith et al. (1992) J. Bacteriol. 174: 5820-26, De Biase et al. (1996) Prot. Exp. Purif. 117: 1411-21 and Turano and Fang (1998) Plant Physiology 8: 430-8). In bacteria and plants, glutamate decarboxylase enzymes (EC 4.1.1.15) are capable of catalyzing the alpha-decarboxylation of glutamic acid to GABA and carbon dioxide. For example, Escherichia coli contains two genes gadA and gadB, which encode the two isozymes GADa and GADb (see, e.g., Smith et al. (1992) J. Bacteriol. 174: 5820-26 and De Biase et al. (1996) Plant Physiology 117: 1411-21). The protein expressed from the two isozymes GADa and GADb are different in five amino-acid residues and have similar functional properties (see, e.g., McCormick and Tunnicliff (2001) Acta Biochem. Pol. 48: 573-78). Glutamate decarboxylase from Streptococcus pneumoniae has been found to exhibit 28% homology with Glutamate decarboxylase 65 from human brain (see, e.g., Garcia and Lopez (1995) FEMS Microbiol. Lett. 133:113-8).

In some embodiments, a glutamic acid catabolism enzyme is encoded by a gene encoding a glutamic acid catabolism enzyme derived from a bacterial species. In some embodiments, a glutamic acid catabolism enzyme is encoded by a gene encoding a glutamic acid catabolism enzyme derived from a non-bacterial species. In some embodiments, a glutamic acid catabolism enzyme is encoded by a gene derived from a eukaryotic species, e.g., a yeast species or a plant species. In one embodiment, the gene encoding the glutamic acid catabolism enzyme is derived from an organism of the genus or species that includes, but is not limited to, Lactococcus, Streptococcus, Escherichia, Arabidopsis, and Thermococcus.

In one embodiment, the glutamic acid catabolism enzyme is a glutamate decarboxylase. As used herein, “glutamate decarboxylase” refers to any polypeptide having enzymatic activity that catalyzes the conversion of glutamic acid to GABA and carbon dioxide. For example, the glutamate decarboxylase enzymes of Escherichia coli (encoded by the gadA and gadB genes) are capable of metabolizing glutamic acid (see, e.g., Smith et al. (1992) J. Bacteriol. 174: 5820-26 and De Biase et al. (1996) Plant Physiology 117: 1411-21). Other distinct glutamate decarboxylase enzymes are also known in the art (see, e.g., U.S. Patent No. CN102911927B, US20020028212, and WO2010/007496, the entire contents of which are expressly incorporated herein by reference in their entireties).

In one embodiment, the glutamate decarboxylase gene is derived from an organism of the genus or species that includes, but is not limited to Streptococcus pneumonia (García and López (1995) FEMS Microbiol. Lett. 133:113-8), Lactococcus lactis (Nomura et al. (1999) Microbiol. 145: 1375-80), Escherichia coli (Smith et al. (1992) J. Bacteriol. 174: 5820-26 and De Biase et al. (1996) Prot. Exp. Purif. 117: 1411-21), Arabidopsis sp. (Turano and Fang (1998) Plant Physiology 8: 430-8), and Thermococcus kodakarensis (Tomita et al. (2014) J. Bacteriol. 196: 1222-30).

In one embodiment, the glutamic acid catabolism enzyme is a glutamate decarboxylase enzyme GadA. In one embodiment, the glutamic acid catabolism enzyme is a glutamate decarboxylase GadB.

In one embodiment, the glutamate decarboxylase gene is gadA gene. In another embodiment, the gadA gene is a Escherichia coli gadA gene. In one embodiment, the glutamate decarboxylase gene is a gadB gene. In another embodiment, the gadB gene is a Escherichia coli gadB gene. In one embodiment, the at least one glutamate decarboxylase gene comprises both a gadA gene and a gadB gene.

In one embodiment, the gadA gene has at least about 80% identity with the sequence of SEQ ID NO:89. Accordingly, in one embodiment, the gadA gene has at least about 90% identity with the sequence of SEQ ID NO:89. Accordingly, in one embodiment, the gadA gene has at least about 95% identity with the sequence of SEQ ID NO:89. Accordingly, in one embodiment, the gadA gene has at least about 85%, 86%, 87%, 88%, 89%, 90%, 91%, 92%, 93%, 94%, 95%, 96%, 97%, 98%, or 99% identity with the sequence of SEQ ID NO:89. In another embodiment, the gadA gene comprises the sequence of SEQ ID NO:89. In yet another embodiment the gadA gene consists of the sequence of SEQ ID NO:89.

In one embodiment, the gadB gene has at least about 80% identity with the sequence of SEQ ID NO:90. Accordingly, in one embodiment, the gadB gene has at least about 90% identity with the sequence of SEQ ID NO:90. Accordingly, in one embodiment, the gadB gene has at least about 95% identity with the sequence of SEQ ID NO:90. Accordingly, in one embodiment, the gadB gene has at least about 85%, 86%, 87%, 88%, 89%, 90%, 91%, 92%, 93%, 94%, 95%, 96%, 97%, 98%, or 99% identity with the sequence of SEQ ID NO:90. In another embodiment, the gadB gene comprises the sequence of SEQ ID NO:90. In yet another embodiment the gadB gene consists of the sequence of SEQ ID NO:90.

The present disclosure further comprises genes encoding functional fragments of a glutamate decarboxylase or functional variants of a glutamate decarboxylase.

Assays for testing the activity of a glutamic acid catabolism enzyme, a glutamic acid catabolism enzyme functional variant, or a glutamic acid catabolism enzyme functional fragment are well known to one of ordinary skill in the art. For example, glutamic acid catabolism can be assessed by expressing the protein, functional variant, or fragment thereof, in a recombinant bacterial cell that lacks endogenous glutamic acid catabolism enzyme activity. Glutamic acid catabolism can be assessed by measuring the activity of glutamate decarboxylase (see, e.g., Yu et al. (2011) Enzy. Microb. Techn. 49:272-6), the entire contents of which are incorporated by reference).

In another embodiment, the gene encoding the glutamate decarboxylase enzyme is dependent on another factor, for example, a glutamate decarboxylase enzyme is dependent on the pyridoxal 5′-phosphate (PLP) co-enzyme.

In one embodiment, the bacterial cell comprises a heterologous gene encoding a glutamic acid catabolism enzyme. In one embodiment, the bacterial cell comprises a heterologous gene encoding a transporter of glutamic acid and a heterologous gene encoding a glutamic acid catabolism enzyme. In one embodiment, the bacterial cell comprises a heterologous gene encoding a glutamic acid catabolism enzyme and a genetic modification that reduces export of glutamic acid. In one embodiment, the bacterial cell comprises a heterologous gene encoding a transporter of glutamic acid, a heterologous gene encoding a glutamic acid catabolism enzyme, and a genetic modification that reduces export of glutamic acid. Transporters and exporters are described in more detail in the subsections, below.

B. Transporters of Glutamic acid

Glutamic acid transporters may be expressed or modified in the recombinant bacteria described herein in order to enhance glutamic acid transport into the cell. Specifically, when thetransporter of glutamic acid is expressed in the recombinant bacterial cells described herein, the bacterial cells import more glutamic acid into the cell when the transporter is expressed than unmodified bacteria of the same bacterial subtype under the same conditions. Thus, the genetically engineered bacteria comprising a heterologous gene encoding transporter of glutamic acid which may be used to import glutamic acid into the bacteria so that any gene encoding an glutamic acid catabolism enzyme expressed in the organism, e.g., co-expressed glutamate decarboxylase, can catabolize the glutamic acid to treat a disease associated with glutamic acid, such as cancer.

The uptake of glutamic acid into bacterial cells is mediated by proteins well known to those of skill in the art. For example, a Na⁺-coupled symporter GltT for glutamic acid uptake has been identified in Bacillus subtilis (see, e.g., Zaprasis et al. (2015) App. Env. Microbiol. 81:250-9). The bacterial gene gltT encodes a glutamic acid transporter responsible for glutamic acid uptake in many bacteria (see, e.g., Jan Slotboom et al. (1999) Microb. Mol. Biol. Rev. 63:293-307; Takahashi et al. (2015) Inf. Imm. 83:3555-67; Ryan et al. (2007) Nat. Struct. Mol. Biol. 14:365-71; and Tolner et al. (1992) Mol. Microbiol. 6:2845-56).

In one embodiment, the at least one gene encoding a transporter of glutamic acid is the gltT gene. In one embodiment, the bacterial cell described herein has been genetically engineered to comprise at least one heterologous gltT gene. In one embodiment, the at least one gene encoding a transporter of glutamic acid is the Escherichia coli gltP gene. In one embodiment, the at least one gene encoding a transporter of glutamic acid is the Bacillus subtilis gltT gene. In one embodiment, the at least one gene encoding a transporter of glutamic acid is the Mycobacterium tuberculosis dctA gene. In one embodiment, the at least one gene encoding a transporter of glutamic acid is the Salmonella typhimurium dctA gene. In one embodiment, the at least one gene encoding a transporter of glutamic acid is the Caenorhabditis elegans gltT gene.

In one embodiment, the gltT gene has at least about 80% identity with the sequence of SEQ ID NO:91. Accordingly, in one embodiment, the gltT gene has at least about 90% identity with the sequence of SEQ ID NO:91. Accordingly, in one embodiment, the gltT gene has at least about 95% identity with the sequence of SEQ ID NO:91. Accordingly, in one embodiment, the gltT gene has at least about 85%, 86%, 87%, 88%, 89%, 90%, 91%, 92%, 93%, 94%, 95%, 96%, 97%, 98%, or 99% identity with the sequence of SEQ ID NO:91. In another embodiment, the gltT gene comprises the sequence of SEQ ID NO:91. In yet another embodiment the gltT gene consists of the sequence of SEQ ID NO:91.

In some embodiments, the transporter of glutamic acid is encoded by a transporter of glutamic acid gene derived from a bacterial genus or species, including but not limited to, Escherichia, Bacillus, Chlamydia, Mycobacterium, Salmonella, Escherichia coli, Mycobacterium tuberculosis, Salmonella typhimurium, or Caenorhabditis elegans (see, e.g., Jan Slotboom et al. (1999) Microbiol. Mol. Biol. Rev. 63:293-307) In some embodiments, the bacterial species is Escherichia coli. In some embodiments, the bacterial species is Escherichia coli strain Nissle.

Assays for testing the activity of a transporter of glutamic acid, a functional variant of a transporter of glutamic acid, or a functional fragment of transporter of glutamic acid are well known to one of ordinary skill in the art. For example, import of glutamic acid may be determined using the methods as described in Zaprasis et al. (2015) App. Env. Microbiol. 81:250-9, the entire contents of each of which are expressly incorporated by reference herein.

In one embodiment, when the transporter of a glutamic acid is expressed in the recombinant bacterial cells described herein, the bacterial cells import 10% more glutamic acid into the bacterial cell when the transporter is expressed than unmodified bacteria of the same bacterial subtype under the same conditions. In another embodiment, when the transporter of glutamic acid is expressed in the recombinant bacterial cells described herein, the bacterial cells import 20%, 30%, 40%, 50%, 60%, 70%, 80%, 90% or 100% more glutamic acid into the bacterial cell when the transporter is expressed than unmodified bacteria of the same bacterial subtype under the same conditions. In yet another embodiment, when the transporter of glutamic acid is expressed in the recombinant bacterial cells described herein, the bacterial cells import two-fold more glutamic acid into the cell when the transporter is expressed than unmodified bacteria of the same bacterial subtype under the same conditions. In yet another embodiment, when the transporter of glutamic acid is expressed in the recombinant bacterial cells described herein, the bacterial cells import three-fold, four-fold, five-fold, six-fold, seven-fold, eight-fold, nine-fold, or ten-fold more glutamic acid into the cell when the transporter is expressed than unmodified bacteria of the same bacterial subtype under the same conditions.

C. Exporters of Glutamic Acid

The export of glutamic acid from bacterial cells is mediated by proteins well known to those of skill in the art. For example, Corynebacterium glutamicum and Escherichia coli were shown to have the ability to export glutamic acid through the proteins MscCG and MscS (encoded by mscS (SEQ ID NO: 92)), respectively (see, e.g., Becker et al. (2013) Bioch. Bioph. Acta 1828: 1230-40).

Glutamic acid exporters may be modified in the recombinant bacteria described herein in order to reduce glutamic acid export from the cell. Specifically, when the recombinant bacterial cells described herein comprise a genetic modification that reduces export of glutamic acid, the bacterial cells retain more glutamic acid in the bacterial cell than unmodified bacteria of the same bacterial subtype under the same conditions. Thus, the recombinant bacteria comprising a genetic modification that reduces export of glutamic acid may be used to retain more glutamic acid in the bacterial cell so that any glutamic acid catabolism enzyme expressed in the organism, e.g., co-expressed glutamate decarboxylase, can catabolize the glutamic acid.

In one embodiment, the genetic modification is a mutation in an endogenous gene encoding an exporter of glutamic acid. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export glutamic acid from the bacterial cell. Assays for testing the activity of an exporter of a glutamic acid are well known to one of ordinary skill in the art.

In another embodiment, the genetic modification is a mutation in a promoter of an endogenous gene encoding an exporter of glutamic acid.

In yet another embodiment, the genetic modification is an overexpression of a repressor of an exporter of glutamic acid. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

14. Histidine

A. Histidine Catabolism Enzymes

Histidine catabolism enzymes may be expressed or modified in the bacteria disclosed herein in order to enhance catabolism of histidine. As used herein, the term “histidine catabolism enzyme” refers to an enzyme involved in the catabolism of histidine. Specifically, when a histidine catabolism enzyme is expressed in a recombinant bacterial cell, the bacterial cell hydrolyzes more histidine into urocanate, or histidine into formamide and glutamate when the catabolism enzyme is expressed than unmodified bacteria of the same bacterial subtype under the same conditions. Thus, the genetically engineered bacteria comprising a heterologous gene encoding an histidine catabolism enzyme can catabolize histidine to treat a disease associated with histidine, such as cancer.

In one embodiment, the histidine catabolism enzyme increases the rate of histidine catabolism in the cell. In one embodiment, the histidine catabolism enzyme decreases the level of histidine in the cell. In another embodiment, the histidine catabolism enzyme increases the level of glutamate in the cell.

Histidine catabolism enzymes are well known to those of skill in the art (see, e.g., Bender (2012) Microbiol. Mol. Biol. Rev. 76: 565-584). In bacteria and eukaryotes, histidase enzymes (EC 4.3.1.3) are capable of eliminating ammonia from histidine as the first step in histidine catabolism. For example, one pathway of histidine catabolism involves the elimination of ammonia from histidine to yield urocanate, hydration of urocanate to give imidazolone propionate (IP), and ring cleavage of IP to yield formiminoglutamate (FIG). In some genera, e.g., Klebsiella and Bacillus, FIG is hydrolyzed to formamide and glutamate, with the formamide being excreted as a waste product (see, e.g., Kaminska et al. (1970) J. Biol. Chem. 245: 3536-3544 and Magasanik and Bowser (1955) J. Biol. Chem. 213: 571-580).

In some embodiments, a histidine catabolism enzyme is encoded by a gene encoding a histidine catabolism enzyme derived from a bacterial species. In some embodiments, a histidine catabolism enzyme is encoded by a gene encoding a histidine catabolism enzyme derived from a non-bacterial species. In some embodiments, a histidine catabolism enzyme is encoded by a gene derived from a eukaryotic species, e.g., a yeast species or a plant species. In one embodiment, the gene encoding the histidine catabolism enzyme is derived from an organism of the genus or species that includes, but is not limited to, Bacillus and Escherichia.

In one embodiment, the histidine catabolism enzyme is a histidine ammonia-lyase enzyme (also known as HutH). As used herein “histidine ammonia-lyase” refers to any polypeptide having enzymatic activity that catalyzes the conversion of histidine to urocanate and ammonia. For example, the histidine ammonia-lyase enzyme HutT is capable of metabolizing histidine, and catalyzes a non-oxidative reaction that liberates the amino group, yielding urocanate as the first intermediate in the pathway (Magasanik and Bowser (1955) J. Biol. Chem. 213: 571-580 and Tabor et al. (1952) J. Biol. Chem. 196: 121-128).

In one embodiment, the histidine ammonia-lyase gene is derived from an organism of the genus or species that includes, but is not limited to enteric bacteria, pseudomonads, Bacillus subtilis (see, e.g., Bender (20126) Microbiology and Molecular Biology reviews. 76: 565-584), and Escherichia coli.

In one embodiment, the histidine ammonia-lyase gene is an hutH gene. In another embodiment, the hutH gene is a Escherichia coli hutH gene. In another embodiment, the hutH gene is a Bacillus amyloliquefaciens hutH gene.

In one embodiment, the hutH gene has at least about 80% identity with the sequence of SEQ ID NO:93. Accordingly, in one embodiment, the hutH gene has at least about 90% identity with the sequence of SEQ ID NO:93. Accordingly, in one embodiment, the hutH gene has at least about 95% identity with the sequence of SEQ ID NO:93. Accordingly, in one embodiment, the hutH gene has at least about 85%, 86%, 87%, 88%, 89%, 90%, 91%, 92%, 93%, 94%, 95%, 96%, 97%, 98%, or 99% identity with the sequence of SEQ ID NO:93. In another embodiment, the hutH gene comprises the sequence of SEQ ID NO:93. In yet another embodiment the hutH gene consists of the sequence of SEQ ID NO:93.

The present disclosure further comprises genes encoding functional fragments of a histidine catabolism enzyme or functional variants of a histidine catabolism enzyme.

Assays for testing the activity of a histidine catabolism enzyme, a histidine catabolism enzyme functional variant, or a histidine catabolism enzyme functional fragment are well known to one of ordinary skill in the art. For example, histidine catabolism can be assessed by expressing the protein, functional variant, or fragment thereof, in a recombinant bacterial cell that lacks endogenous histidine catabolism enzyme activity. Histidine catabolism can be assessed using the histidine ammonia-lyase assay method (see, e.g., Hassall, H. (1971) Methods in Enzymology, XVII B, 895-897; or Shin et al. (1983) Journal of Inherited Metabolic Disease 6: 113-114).

In one embodiment, the gene encoding the histidine catabolism enzyme is a histidine ammonia-lyase gene. In another embodiment, the gene encoding the histidine ammonia-lyase co-expressed with an additional histidine catabolism enzyme, for example, an formimino glutamate deiminase enzyme.

In one embodiment, the bacterial cell comprises a heterologous gene encoding a histidine catabolism enzyme. In one embodiment, the bacterial cell comprises a heterologous gene encoding a transporter of histidine and a heterologous gene encoding a histidine catabolism enzyme. In one embodiment, the bacterial cell comprises a heterologous gene encoding a histidine catabolism enzyme and a genetic modification that reduces export of histidine. In one embodiment, the bacterial cell comprises a heterologous gene encoding a transporter of histidine, a heterologous gene encoding a histidine catabolism enzyme, and a genetic modification that reduces export of histidine. Transporters and exporters are described in more detail in the subsections, below.

B. Transporters of Histidine

Histidine transporters may be expressed or modified in the recombinant bacteria described herein in order to enhance histidine transport into the cell. Specifically, when the transporter of histidine is expressed in the recombinant bacterial cells described herein, the bacterial cells import more histidine into the cell when thetransporter is expressed than unmodified bacteria of the same bacterial subtype under the same conditions. Thus, the genetically engineered bacteria comprising a heterologous gene encoding a transporter of histidine which may be used to import histidine into the bacteria so that any gene encoding a histidine catabolism enzyme expressed in the organism, e.g., co-expressed histidine ammonia-lyase, can catabolize the histidine to treat a disease associated with amino acid metabolism, such as cancer.

The uptake of histidine into bacterial cells is mediated by proteins well known to those of skill in the art. For example, a histidine transport system is encoded by the hisJQMP operon and the artJ gene (see, e.g., Caldara et al. (2007) J. Mol. Biol. 373(2): 251-67). Transport by the histidine transport system is mediated by several proteins regulated by the ArgR-L-arginine DNA-binding transcriptional dual regulator. ArgR complexed with L-arginine represses the transcription of several genes involved in transport of histidine. In this system, HisJ (encoded by hisJ) is an histidine ABC transporter—periplasmic binding protein, HisQ and HisM (encoded by hisQ and hisM respectively) are lysine/arginine/ornithine ABC transporter/histidine ABC transporter—membrane subunit, HisP (encoded by hisP) is a lysine/arginine/ornithine ABC transporter/histidine ABC transporter—ATP binding subunit. This system has been well characterized in Escherichia coli (see, e.g., Caldara et al. (2007) J. Mol. Biol. 373(2): 251-67). In addition, bacterial systems that are homologous and orthologous of the E. coli histidine-specific system have been characterized in other bacterial species, including, for example, Pseudomonas fluorescens (see, e.g., M. Bender (20126) Microbiology and Molecular Biology reviews. 76: 565-584). These membranous and membrane-associated proteins of the histidine permease (Q M P complex), encoded by the hisJQMP operon, resulting in the uptake of histidine (see, e.g., Oh et al. (1994) J. Biol. Chem. 269(42): 26323-30).

In one embodiment, the at least one gene encoding a transporter of histidine comprises the hisJQMP operon. In one embodiment, the at least one gene encoding a transporter of histidine comprises the hisJ gene. In one embodiment, the at least one gene encoding a transporter of histidine comprises the hisQ gene. In one embodiment, the at least one gene encoding a transporter of histidine comprises the hisM gene. In one embodiment, the at least one gene encoding a transporter of histidine comprises the hisP gene.

In one embodiment, the hisJ gene has at least about 80% identity with the sequence of SEQ ID NO:94. Accordingly, in one embodiment, the hisJ gene has at least about 90% identity with the sequence of SEQ ID NO:94. Accordingly, in one embodiment, the hisJ gene has at least about 95% identity with the sequence of SEQ ID NO:94. Accordingly, in one embodiment, the hisJ gene has at least about 85%, 86%, 87%, 88%, 89%, 90%, 91%, 92%, 93%, 94%, 95%, 96%, 97%, 98%, or 99% identity with the sequence of SEQ ID NO:94. In another embodiment, the hisJ gene comprises the sequence of SEQ ID NO:94. In yet another embodiment, the hisJ gene consists of the sequence of SEQ ID NO:94.

In one embodiment, the hisQ gene has at least about 80% identity with the sequence of SEQ ID NO:95. Accordingly, in one embodiment, the hisQ gene has at least about 90% identity with the sequence of SEQ ID NO:95. Accordingly, in one embodiment, the hisQ gene has at least about 95% identity with the sequence of SEQ ID NO:95. Accordingly, in one embodiment, the hisQ gene has at least about 85%, 86%, 87%, 88%, 89%, 90%, 91%, 92%, 93%, 94%, 95%, 96%, 97%, 98%, or 99% identity with the sequence of SEQ ID NO:95. In another embodiment, the hisQ gene comprises the sequence of SEQ ID NO:95. In yet another embodiment, the hisQ gene consists of the sequence of SEQ ID NO:95.

In one embodiment, the hisM gene has at least about 80% identity with the sequence of SEQ ID NO:103. Accordingly, in one embodiment, the hisM gene has at least about 90% identity with the sequence of SEQ ID NO:103. Accordingly, in one embodiment, the hisM gene has at least about 95% identity with the sequence of SEQ ID NO:103. Accordingly, in one embodiment, the hisM gene nhas at least about 85%, 86%, 87%, 88%, 89%, 90%, 91%, 92%, 93%, 94%, 95%, 96%, 97%, 98%, or 99% identity with the sequence of SEQ ID NO:103. In another embodiment, the hisM gene comprises the sequence of SEQ ID NO:103. In yet another embodiment, the hisM gene consists of the sequence of SEQ ID NO:103.

In one embodiment, the hisP gene has at least about 80% identity with the sequence of SEQ ID NO:96. Accordingly, in one embodiment, the hisP gene has at least about 90% identity with the sequence of SEQ ID NO:96. Accordingly, in one embodiment, the hisP gene has at least about 95% identity with the sequence of SEQ ID NO:96. Accordingly, in one embodiment, the hisP gene nhas at least about 85%, 86%, 87%, 88%, 89%, 90%, 91%, 92%, 93%, 94%, 95%, 96%, 97%, 98%, or 99% identity with the sequence of SEQ ID NO:96. In another embodiment, the hisP gene comprises the sequence of SEQ ID NO:96. In yet another embodiment, the hisP gene consists of the sequence of SEQ ID NO:96.

In some embodiments, the transporter of histidine is encoded by a transporter of histidine gene derived from a bacterial genus or species, including but not limited to, Escherichia and Pseudomonas In some embodiments, the bacterial species is Escherichia coli. In some embodiments, the bacterial species is Escherichia coli strain Nissle.

Assays for testing the activity of a transporter of histidine, a functional variant of a transporter of histidine, or a functional fragment of transporter of histidine are well known to one of ordinary skill in the art. For example, import of histidine may be determined using the methods as described in Liu et al (1997) J. Biol. Chem. 272: 859-866 or Shang et al (2013) J. Bacteriology. 195(23): 5334-5342., the entire contents of each of which are expressly incorporated by reference herein.

In one embodiment, when the transporter of a histidine is expressed in the recombinant bacterial cells described herein, the bacterial cells import 10% more histidine into the bacterial cell when the transporter is expressed than unmodified bacteria of the same bacterial subtype under the same conditions. In another embodiment, when the transporter of histidine is expressed in the recombinant bacterial cells described herein, the bacterial cells import 20%, 30%, 40%, 50%, 60%, 70%, 80%, 90% or 100% more histidine into the bacterial cell when the transporter is expressed than unmodified bacteria of the same bacterial subtype under the same conditions. In yet another embodiment, when the transporter of histidine is expressed in the recombinant bacterial cells described herein, the bacterial cells import two-fold more histidine into the cell when the transporter is expressed than unmodified bacteria of the same bacterial subtype under the same conditions. In yet another embodiment, when the transporter of histidine is expressed in the recombinant bacterial cells described herein, the bacterial cells import three-fold, four-fold, five-fold, six-fold, seven-fold, eight-fold, nine-fold, or ten-fold more histidine into the cell when the transporter is expressed than unmodified bacteria of the same bacterial subtype under the same conditions.

C. Exporters of Histidine

Histidine exporters may be modified in the recombinant bacteria described herein in order to reduce histidine export from the cell. Specifically, when the recombinant bacterial cells described herein comprise a genetic modification that reduces export of histidine, the bacterial cells retain more histidine in the bacterial cell than unmodified bacteria of the same bacterial subtype under the same conditions. Thus, the recombinant bacteria comprising a genetic modification that reduces export of histidine may be used to retain more histidine in the bacterial cell so that any histidine catabolism enzyme expressed in the organism, e.g., co-expressed histidine ammonia-lyase, can catabolize the histidine.

The export of histidine from bacterial cells is mediated by proteins well known to those of skill in the art. For example, Corynebacterium glutamicum was shown to have the ability to export histidine, which may allow to maintain histidine homoeostasis in an environment rich in histidine-containing peptides (see, e.g., Bellmann et al. (2001) Microbiology 147:1765-1774). Assays for testing the activity of an exporter of a histidine are also well known to one of ordinary skill in the art. For example, export of histidine may be determined using the methods described by Bellmann et al. (2001) Microbiology 147: 1765-74), the entire contents of which are expressly incorporated herein by reference.

In one embodiment, the genetic modification is a mutation in an endogenous gene encoding an exporter of histidine. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export histidine from the bacterial cell. Assays for testing the activity of an exporter of a histidine are well known to one of ordinary skill in the art.

In another embodiment, the genetic modification is a mutation in a promoter of an endogenous gene encoding an exporter of histidine.

In yet another embodiment, the genetic modification is an overexpression of a repressor of an exporter of histidine. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

15. Proline

A. Proline Catabolism Enzymes

Proline catabolism enzymes may be expressed or modified in the bacteria disclosed herein in order to enhance catabolism of proline. As used herein, the term “proline catabolism enzyme” refers to an enzyme involved in the catabolism of proline. Specifically, when an proline catabolism enzyme is expressed in a recombinant bacterial cell, the bacterial cell hydrolyzes more proline into 5-aminovalerate when the catabolism enzyme is expressed than unmodified bacteria of the same bacterial subtype under the same conditions. Thus, the genetically engineered bacteria comprising a heterologous gene encoding an proline catabolism enzyme can catabolize proline to treat a disease associated with proline, such as cancer.

In one embodiment, the proline catabolism enzyme increases the rate of proline catabolism in the cell. In one embodiment, the proline catabolism enzyme decreases the level of proline in the cell. In another embodiment, the proline catabolism enzyme increases the level of 5-aminovalerate in the cell.

Proline catabolism enzymes are well known to those of skill in the art (see, e.g., Kabisch et al. (1999) J. Biol. Chem. 274: 8445-54). For example, in bacteria of the genus Clostridium proline reductases (EC 1.4.1.6) catalyze the reductive ring cleavage of _{D}-proline to 5-aminovalerate.

In some embodiments, an proline catabolism enzyme is encoded by a gene encoding an proline catabolism enzyme derived from a bacterial species. In some embodiments, an proline catabolism enzyme is encoded by a gene encoding an proline catabolism enzyme derived from a non-bacterial species. In some embodiments, an proline catabolism enzyme is encoded by a gene derived from a eukaryotic species, e.g., a yeast species or a plant species. In one embodiment, the gene encoding the proline catabolism enzyme is derived from an organism of the genus or species that includes, but is not limited to, Clostridium botulinum and Clostridium sticklandii.

In one embodiment, the proline catabolism enzyme is a proline reductase. As used herein, “proline reductase” refers to any polypeptide having enzymatic activity that catalyzes the conversion of proline to 5-aminovalerate. For example, the proline reductase PrdA of Clostridium sticklandii (encoded by the prdA gene) is capable of metabolizing proline (see, e.g., Kabisch et al. (1999) J. Biol. Chem. 274: 8445-54).

In one embodiment, the proline reductase gene is a prdA gene. In one embodiment, the proline reductase gene is an Clostridum sticklandii prdA gene. In one embodiment, the proline reductase gene is an Clostridum botulinum prdA gene.

In one embodiment, the prdA gene has at least about 80% identity with the sequence of SEQ ID NO:97. Accordingly, in one embodiment, the prdA gene has at least about 90% identity with the sequence of SEQ ID NO:97. Accordingly, in one embodiment, the ansA gene has at least about 95% identity with the sequence of SEQ ID NO:97. Accordingly, in one embodiment, the prdA gene has at least about 85%, 86%, 87%, 88%, 89%, 90%, 91%, 92%, 93%, 94%, 95%, 96%, 97%, 98%, or 99% identity with the sequence of SEQ ID NO:97. In another embodiment, the prdA gene comprises the sequence of SEQ ID NO:97. In yet another embodiment the prdA gene consists of the sequence of SEQ ID NO:97.

The present disclosure further comprises genes encoding functional fragments of a proline catabolism enzyme or functional variants of a proline catabolism enzyme.

Assays for testing the activity of an proline catabolism enzyme, a proline catabolism enzyme functional variant, or an proline catabolism enzyme functional fragment are well known to one of ordinary skill in the art. For example, proline catabolism can be assessed by expressing the protein, functional variant, or fragment thereof, in a recombinant bacterial cell that lacks endogenous proline catabolism enzyme activity. Proline catabolism can be assessed as described in Kabisch et al. (1999) J. Biol. Chem. 274: 8445-54, the entire contents of which are incorporated by reference.

In one embodiment, the bacterial cell comprises a heterologous gene encoding an proline catabolism enzyme. In one embodiment, the bacterial cell comprises a heterologous gene encoding a transporter of proline and a heterologous gene encoding an proline catabolism enzyme. In one embodiment, the bacterial cell comprises a heterologous gene encoding an proline catabolism enzyme and a genetic modification that reduces export of proline. In one embodiment, the bacterial cell comprises a heterologous gene encoding a transporter of proline, a heterologous gene encoding an proline catabolism enzyme, and a genetic modification that reduces export of proline. Transporters and exporters are described in more detail in the subsections, below.

B. Transporters of Proline

Proline transporters may be expressed or modified in the recombinant bacteria described herein in order to enhance proline transport into the cell. Specifically, when the transporter of proline is expressed in the recombinant bacterial cells described herein, the bacterial cells import more proline into the cell when the transporter is expressed than unmodified bacteria of the same bacterial subtype under the same conditions. Thus, the genetically engineered bacteria comprising a heterologous gene encoding transporter of proline which may be used to import proline into the bacteria so that any gene encoding an proline catabolism enzyme expressed in the organism can catabolize the proline to treat a disease associated with proline, such as cancer.

The uptake of proline into bacterial cells is mediated by proteins well known to those of skill in the art. The proline utilization operon (put) allows bacterial cells to transport and use proline. The put operon consists of two genes putA and putP. In bacteria, there are two distinct systems for proline uptake, proline porter I (PPI) and proline porter II (PPII) (see, e.g., Grothe (1986) J. Bacteriol. 166: 253-259). The bacterial gene putP encodes a proline transporter responsible for proline uptake in many bacteria (see, e.g., Ostrovsky et al. (1993) Proc. Natl. Acad. Sci. 90: 429-8; Grothe (1986) J. Bacteriol. 166: 253-259). The putA gene expresses a polypeptide that has proline dehydrogenase (EC 1.5.99.8) activity and pyrroline-5-carboxylate (P5C) (EC 1.5.1.12) activity (see, e.g., Menzel and Roth (1981) J. Biol. Chem. 256:9755-61). In the absence of proline, putA remains in the cytoplasm and represses put gene expression. In the presence of proline, putA binds to the membrane relieving put repression allowing put gene expression (see, e.g., Ostrovsky et al. (1993) Proc. Natl. Acad. Sci. 90: 429-8).

In one embodiment, the at least one gene encoding the transporter of proline is the putP gene. In one embodiment, the bacterial cell described herein has been genetically engineered to comprise at least one heterologous putP gene. In one embodiment, the at least one gene encoding the transporter of proline is the Escherichia coli putP gene. In one embodiment, the at least one gene encoding the transporter of proline is the Salmonella typhimurium putP gene. In one embodiment, the at least one gene encoding a transporter of proline is the Escherichia coli putP gene.

In one embodiment, the putP gene has at least about 80% identity with the sequence of SEQ ID NO:98. Accordingly, in one embodiment, the putP gene has at least about 90% identity with the sequence of SEQ ID NO:98. Accordingly, in one embodiment, the putP gene has at least about 95% identity with the sequence of SEQ ID NO:98. Accordingly, in one embodiment, the putP gene has at least about 85%, 86%, 87%, 88%, 89%, 90%, 91%, 92%, 93%, 94%, 95%, 96%, 97%, 98%, or 99% identity with the sequence of SEQ ID NO:98. In another embodiment, the putP gene comprises the sequence of SEQ ID NO:98. In yet another embodiment the putP gene consists of the sequence of SEQ ID NO:98.

In some embodiments, the transporter transporter of proline is encoded by a transporter of proline gene derived from a bacterial genus or species, including but not limited to, Escherichia, Salmonella, Escherichia coli or Salmonella typhimurium. In some embodiments, the bacterial species is Escherichia coli. In some embodiments, the bacterial species is Escherichia coli strain Nissle.

Assays for testing the activity of a transporter transporter of proline, a functional variant of a transporter transporter of proline, or a functional fragment of a transporter of proline are well known to one of ordinary skill in the art. For example, import of proline may be determined using the methods as described in Moses et al. (2012) Journal of Bacteriology 194: 745-58 and Hoffman et al. (2012) App. and Enviro. Microbiol. 78: 5753-62), the entire contents of each of which are expressly incorporated by reference herein.

In one embodiment, when the transporter of a proline is expressed in the recombinant bacterial cells described herein, the bacterial cells import 10% more proline into the bacterial cell when the transporter is expressed than unmodified bacteria of the same bacterial subtype under the same conditions. In another embodiment, when the transporter of proline is expressed in the recombinant bacterial cells described herein, the bacterial cells import 20%, 30%, 40%, 50%, 60%, 70%, 80%, 90% or 100% more proline into the bacterial cell when the transporter is expressed than unmodified bacteria of the same bacterial subtype under the same conditions. In yet another embodiment, when the transporter of proline is expressed in the recombinant bacterial cells described herein, the bacterial cells import two-fold more proline into the cell when the transporter is expressed than unmodified bacteria of the same bacterial subtype under the same conditions. In yet another embodiment, when the transporter of proline is expressed in the recombinant bacterial cells described herein, the bacterial cells import three-fold, four-fold, five-fold, six-fold, seven-fold, eight-fold, nine-fold, or ten-fold more proline into the cell when the transporter is expressed than unmodified bacteria of the same bacterial subtype under the same conditions.

C. Exporters of Proline

Proline exporters may be modified in the recombinant bacteria described herein in order to reduce proline export from the cell. Specifically, when the recombinant bacterial cells described herein comprise a genetic modification that reduces export of proline, the bacterial cells retain more proline in the bacterial cell than unmodified bacteria of the same bacterial subtype under the same conditions. Thus, the recombinant bacteria comprising a genetic modification that reduces export of proline may be used to retain more proline in the bacterial cell so that any proline catabolism enzyme expressed in the organism can catabolize the proline.

In one embodiment, the genetic modification is a mutation in an endogenous gene encoding an exporter of proline. In another embodiment, the genetic mutation results in an exporter having reduced activity as compared to a wild-type exporter protein. In one embodiment, the activity of the exporter is reduced at least 50%, at least 75%, or at least 100%. In another embodiment, the activity of the exporter is reduced at least two-fold, three-fold, four-fold, or five-fold. In another embodiment, the genetic mutation results in an exporter having no activity and which cannot export proline from the bacterial cell. Assays for testing the activity of an exporter of a proline are well known to one of ordinary skill in the art.

In another embodiment, the genetic modification is a mutation in a promoter of an endogenous gene encoding an exporter of proline.

In yet another embodiment, the genetic modification is an overexpression of a repressor of an exporter of proline. In one embodiment, the overexpression of the repressor of the exporter is caused by a mutation which renders the promoter of the repressor constitutively active. In another embodiment, the overexpression of the repressor of the exporter is caused by the insertion of an inducible promoter in front of the repressor so that the expression of the repressor can be induced. Inducible promoters are described in more detail herein.

Generation of Bacterial Strains with Enhance Ability to Transport Amino Acids

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing an amino acid is disrupted a strain capable of high-affinity capture of said amino acid can be evolved via ALE. First, the strain is grown in varying concentrations of the auxotrophic amino acid, until a minimum concentration to support growth is established. The strain is then passaged at that concentration, and diluted into lowering concentrations of the amino acid at regular intervals. Over time, cells that are most competitive for the amino acid—at growth-limiting concentrations—will come to dominate the population. These strains will likely have mutations in their amino acid-transporters resulting in increased ability to import the essential and limiting amino acid.

Similarly, by using an auxotroph that cannot use an upstream metabolite to form an amino acid, a strain can be evolved that not only can more efficiently import the upstream metabolite, but also convert the metabolite into the essential downstream metabolite. These strains will also evolve mutations to increase import of the upstream metabolite, but may also contain mutations which increase expression or reaction kinetics of downstream enzymes, or that reduce competitive substrate utilization pathways.

A metabolite innate to the microbe can be made essential via mutational auxotrophy and selection applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate.

Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

Similar methods can be used to generate E. Coli Nissle mutants that consume or import amino acids.

Tryptophan

A. Tryptophan

1-Tryptophan (TRP) is one of the nine essential amino acids and is the least abundant of all 21 dietary amino acids in human beings. Dietary TRP is transported from the digestive tract through the portal vein to the liver where it is used for the synthesis of proteins. The distinguishing structural characteristic of TRP is that it contains an indole functional group. Apart from protein synthesis, TRP is used in the generation of products such as serotonin, melatonin, tryptamine, and the products of the kynurenine pathway (KP, collectively called the kynurenines). TRP and its catabolites have well characterized immunosuppressive and disease tolerance functions, and contribute to immune privileged sites such as eyes, brain, placenta, and testes. The kynurenine pathway represents >95% of TRP-catabolizing pathways and is now established as a key regulator of innate and adaptive immunity through its involvement in cancer, autoimmunity, and infection.

Several KP Pathway metabolites, most notably kynurenine, have been shown to be activating ligands for the arylcarbon receptor (AhR; also known as dioxin receptor). Kynurenine (KYN) was initially shown in the cancer setting as an endogenous AHR ligand in immune and tumor cells, acting both in an autocrine and paracrine manner, and promoting tumor cell survival.

In the gut, e kynurenine pathway metabolism is regulated by gut microbiota, which can regulate tryptophan availability for kynurenine pathway metabolism. Tryptophan may be transported across the epithelium by transport machinery comprising angiotensin I converting enzyme 2 (ACE2), and converted to kynurenine, where it functions in the suppression of T cell responses and promotion of Treg cells.

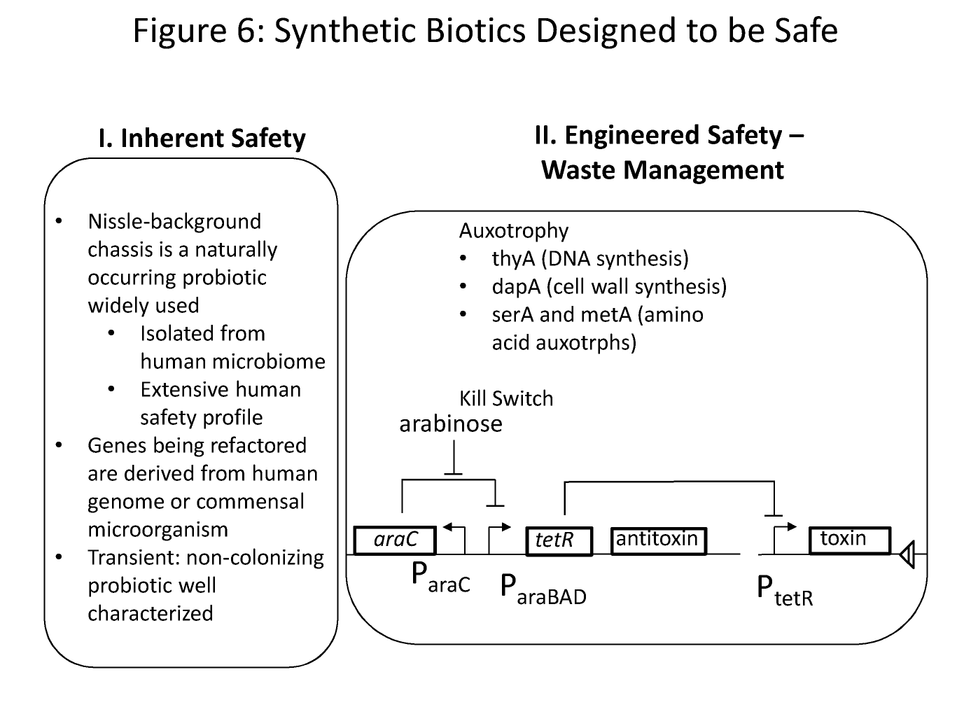

More recently, additional tryptophan metabolites, collectively termed “indoles”, herein, also have been shown to function as AhR agonists. The metabolites include for example, indole-3 aldehyde, indole-3 acetate, indole-3 propionic acid, indole, indole-3 acetaladehyde, indole-3acetonitrile, FICZ, etc., and tryptamine (are, see e.g., Table 8 and FIGS. 6A and 6B and elsewhere herein, and Lama et al., Nat Med. 2016 June; 22(6):598-605; CARDS impacts colitis by altering gut microbiota metabolism of tryptophan into aryl hydrocarbon receptor ligands). The majority of these metabolites are generated by the microbiota; some are generated by the human host and/or taken up from the diet.

Ahr best known as a receptor for xenobiotics such as polycyclic aromatic hydrocarbons AhR is a ligand-dependent cytosolic transcription factor that is able to translocate to the cell nucleus after ligand binding. The in addition to kynurenine, other tryptophan metabolites, e.g., indoles (described herein, tryptamine, and kynurenic acide (KYNA) have recently been identified as endogenous AhR ligands mediating immunosuppressive functions. To induce transcription of AhR target genes in the nucleus, AhR partners with proteins such as AhR nuclear translocator (ARNT) or NF-κB subunit RelB. Studies on human cancer cells have shown that KYN activates the AhR-ARNT associated transcription of IL-6, which induced autocrine activation of IDO1 via STAT3. This AhR-IL-6-STAT3 loop is associated with a poor prognosis in lung cancer, supporting the idea that IDO/kynurenine-mediated immunosuppression enables the immune escape of tumor cells.

B. Kynurenine Pathway

Kynurenine, IDO, and TDO

The rate-limiting conversion of tryptophan to kynurenine (KYN) may be mediated by either of two forms of indoleamine 2, 3-dioxygenase, IDO1 expressed ubiquitously, IDO2 expressed in kidneys, epididymis, testis, and liver or by tryptophan 2,3-dioxygenase (TDO) expressed in the liver and brain. IDO1 expression is specifically induced by inflammatory stimuli, such as the cytokines TNF-α or IFN-γ. IDO1 activity by professional antigen presenting cells reduces local tryptophan concentrations and elevates toxic kynurenine metabolites to limit activated T-cell responses and promote regulatory Tcell activity, e.g. mediated through AhR signaling.

TDO is essential for homeostasis of TRP concentrations and has a lower affinity to TRP than IDO1. Its expression is activated mainly by increased plasma TRP concentrations but can also be activated by glucocorticoids and glucagon.

The tryptophan kynurenine pathway is also expressed in a large number of microbiota, most prominently in Enterobacteriaceae, and kynurenine and metabolites may be synthesized in the gut and Sci Transl Med. 2013 Jul. 10; 5(193): 193ra91). In some embodiments, the genetically engineered bacteria comprise one or more heterologous bacterially derived genes from Enterobacteriaceae, e.g. whose gene products catalyze the conversion of TRP:KYN.

In one embodiment, the genetically engineered bacteria comprise any suitable gene for producing kynurenine. In some embodiments, the genetically engineered bacteria may comprise a gene or gene cassette for producing a tryptophan transporter, a gene or gene cassette for producing IDO-1, and a gene or gene cassette for producing TDO. In some embodiments, the gene for producing kynurenine is modified and/or mutated, e.g., to enhance stability, increase kynurenine production, and/or increase anti-inflammatory potency under inducing conditions. In some embodiments, the engineered bacteria have enhanced uptake or import of tryptophan, e.g., comprise a transporter or other mechanism for increasing the uptake of tryptophan into the bacterial cell. In some embodiments, the genetically engineered bacteria are capable of producing kynurenine under inducing conditions, e.g., under a condition(s) associated with inflammation. In some embodiments, the genetically engineered bacteria are capable of producing kynurenine in low-oxygen conditions. In some embodiments the genetically engineered bacteria secrete an enzyme which produces kynurenine.

Post-Kynurenine Metabolism

As shown in FIG. XXX, kynurenine is further metabolized along the two distinct routes competing for kynurenine as a substrate: (a) KYN, kynurenic acid (KYNA) pathway; and (b) KYN, nicotinamide adenine dinucleotide (NAD) pathway.

a. Kynurenic Acid, Xanthurenic Acid, Anthranillic Acid

Kynurenine is further metabolized along the two distinct routes competing for KYN as a substrate: (a) KYN, kynurenic acid (KYNA) pathway; and (b) KYN, nicotinamide adenine dinucleotide (NAD) pathway

Along one arm, KYN may be further metabolized to another bioactive metabolite, kynurenic acid, (KYNA).

KYNA is generated by kynurenine aminotransferases (KAT I, II, III), e.g., in astrocytes in the brain and can also bind AHR and GPCRs, e.g., GPR35, glutamate receptors, N-methyl D-aspartate (NMDA)-receptors. Elevated levels of KYNA have previously been observed in patients with schizophrenia, both in the cerebrospinal fluid (CSF) and postmortem prefrontal cortex.

The major nerve supply to the gut is also activated the activation of NMDA glutamate receptors in the major nerve supply to the GI tract (i.e., the myenteric plexus) leads to an increase in gut motility (Forrest et al., 2003), but rats treated with kynurenic acid exhibit decreased gut motility and inflammation in the early phase of acute colitis (Varga et al., 2010). Thus, the elevated levels of kynurenic acid reported in IBD patients may represent a compensatory response to the increased activation of enteric neurons (Forrest et al., 2003).

KYNA also has signaling functions through activation of its recently identified receptor, GPR35. GPR35 is predominantly detected in immune cells in the gastrointestinal tract, and might be involved in nociceptive perception. KYNA might have an anti-inflammatory effect by inhibition of lipopolysaccharide-induced tumor necrosis factor (TNF)-alpha secretion in peripheral blood mononuclear cells.

Additionally, KYNA has been found to be generated by macrophages. Increased concentrations of KYNA and xanthurenic acid (3-Hydroxy KYNA, XA) were detected in the plasma of patients with type 2 diabetes, presumably due to chronic stress or the low-grade inflammation that are prominent risk factors for diabetes. Thermochemical and kinetic data show that KYNA and XA are the best free-radical scavengers from the eight tested TRP metabolites, suggesting that the production is a regulatory mechanism to attenuate damage by the inflammation-induced production of reactive oxygen species.

The genetically engineered bacteria may comprise any suitable gene for producing kynurenic acid. In some embodiments, the genetically engineered bacteria are capable of producing kynurenic acid, e.g., from kynurenine through a circuit comprising gene(s) or gene sequence(s) comprising kynurenine-oxoglutarate transaminase or an equivalent thereof.

In some embodiments, the gene for producing kynurenic acid is modified and/or mutated, e.g., to enhance stability, increase kynurenic acid production, and/or increase anti-inflammatory potency under inducing conditions. In some embodiments, the genetically engineered bacteria are capable of producing kynurenic acid under inducing conditions, e.g., under a condition(s) associated with inflammation. In some embodiments, the genetically engineered bacteria are capable of producing kynurenic acid in low-oxygen conditions. IN some embodiments, the genetically engineered bacteria secrete an enzyme for the production of kynurenic acid.

b. The KYN-Nicotinamide Adenine Dinucleotide Pathway

The major enzymes of the KYN-NAD pathway are KYN-3-monooxygenase and kynureninase. Among more than 30 intermediate metabolites (collectively named “kynurenines”) are NMDA agonists (quinolinic and picolinic acids) and free radical generators (3-hydroxykynurenine and 3-hydroxyanthranilic acids). One of the major metabolites of this pathway, 3-hydroxykynurenine (3-HK), is a potential neurotoxin involved in several neurodegenerative disorders. The other metabolite, xanthurenic acid, reacts with insulin with formation of a complex antigenetically indistinguishable from insulin. Quinolinic acid (a glutamate receptor agonist) and picolinic acids exert anxiogenic (anxiety causing) effects in animal models, and play a neurotoxic role. Quinolinic and picolinic acids stimulate inducible nitric oxide synthase (iNOS and together with 3-hydroxykynurenine and 3-hydroxyanthranilic acids might increase lipid peroxidation, and trigger an arachidonic acid cascade resulting in the increased production of inflammatory factors.

Anthranilic and xanthurenic acid can act as antioxidants in certain chemical environments. Patients suffering from neurological disorders as Huntington's disease or brain injury often showed decreased levels of xanthurenic acid combined with increased levels of anthranilic acid (AA). However, the biological importance of the 3-hydroxyanthranilic acid (3-HA) to AA ratio as either neurotoxic or neuroprotective mechanism is still discussed.

Therefore, finding a means to upregulate and/or downregulate the levels of flux through the KP and to reset relative amounts and/or ratios of tryptophan and its various bioactive metabolites may be useful in the prevention, treatment and/or management of a number of diseases as described herein. The present disclosure describes compositions for modulating, regulating and fine tuning tryptophan and tryptophan metabolite levels, e.g., in the serum or in the gastrointestinal system, through genetically engineered bacteria which comprise circuitry enabling the synthesis, bacterial uptake and catabolism of tryptophan and/or tryptophan metabolites, and provides methods for using these compositions in the treatment, management and/or prevention of a number of different diseases.

In certain embodiments the genetically engineered bacteria comprise one or more genes(s) or gene cassettes, which can synthesize tryptophan and/or one or more of its metabolites, thereby modulating local and/or systemic concentrations and or ratios of tryptophan and/or one or more of its metabolites.

In some embodiments, the genetically engineered bacteria modulate the inflammatory status, influence immunosuppression, disease tolerance, or neurological status.

C. Other Indole Tryptophan Metabolites

In addition to kynurenine and KYNA, numerous compounds have been proposed as endogenous AHR ligands, many of which are generated through pathways involved in the metabolism of tryptophan and indole (Bittinger et al., 2003; Chung and Gadupudi, 2011) A large number of metabolites generated through the tryptophan indole pathway are generated by microbiota in the gut. For example, bacteria take up tryptophan, which can be converted to mono-substituted indole compounds, such as indole acetic acid (IAA) and tryptamine, and other compounds, which have been found to activate the AHR (Hubbard et al., 2015, Adaptation of the human aryl hydrocarbon receptor to sense microbiota-derived indoles; Nature Scientific Reoports 5:12689).

In the gastronintestinal tract, diet derived and bacterially AhR ligands promote IL-22 production by innate lymphoid cells, referred to as group 3 ILCs (Spits et al., 2013, Zelante et al., Tryptophan Catabolites from Microbiota Engage Aryl Hydrocarbon Receptor and Balance Mucosal Reactivity via Interleukin-22; Immunity 39, 372-385, Aug. 22, 2013). AHR is essential for IL-22-production in the intestinal lamina propria (Lee et al., Nature Immunology 13, 144-151 (2012); AHR drives the development of gut ILC22 cells and postnatal lymphoid tissues via pathways dependent on and independent of Notch).

Through initiation of Jak-STAT signaling pathways, IL-22 expression can trigger expression of antimicrobial compounds as well as a range of cell growth related pathways, both of which enhance tissue repair mechanisms. IL-22 is critical in promoting intestinal barrier fidelity and healing, while modulating inflammatory states. Murine models have demonstrated improved intestinal inflammation states following administration of IL-22. Additionally, IL-22 activates STAT3 signaling to promote enhanced mucus production to preserve barrier function.

Table 8 lists exemplary tryptophan metabolites which have been shown to bind to AhR and which can be produced by the genetically engineered bacteria of the disclosure.

TABLE 8.
i. Indole Tryptophan Metabolites

| Origin | Compound |
|---|---|
| Exogenous | 2,3,7,8-Tetrachlorodibenzo-p-dioxin (TCDD) |
| Dietary | Indole-3-carbinol (I3C) |
| Dietary | Indole-3-acetonitrile (I3ACN) |
| Dietary | 3.3′-Diindolylmethane (DIM) |
| Dietary | 2-(indol-3-ylmethyl)-3.3′-diindolylmethane (Ltr-1) |
| Dietary | Indolo(3,2-b)carbazole (ICZ) |
| Dietary | 2-(1′H-indole-3′-carbony)-thiazole-4- |
| | carboxylic acid methyl ester (ITE) |
| Microbial | Indole |
| Microbial | Indole-3-acetic acid (IAA) |
| Microbial | Indole-3-aldehyde (IAId) |
| Microbial | Tryptamine |
| Microbial | 3-methyl-indole (Skatole) |
| Yeast | Tryptanthrin |
| Microbial/Host | Indigo |
| Metabolism | |
| Microbial/Host | Indirubin |
| Metabolism | |
| Microbial/Host | Indoxyl-3-sulfate (I3S) |
| Metabolism | |
| Host | Kynurenine (Kyn) |
| Metabolism | |
| Host | Kynurenic acid (KA) |
| Metabolism | |
| Host | Xanthurenic acid |
| Metabolism | |
| Host | Cinnabarinic acid (CA) |
| Metabolism | |
| UV-Light | 6-formylindolo(3,2-b)carbazole (FICZ) |
| Oxidation | |
| Microbial | |
| metabolism | |

In addition, some indole metabolites may exert their effect through Pregnane X receptor (PXR), which is thought to play a key role as an essential regulator of intestinal barrier function. PXR-deficient (Nrli2−/−) mice showed a distinctly “leaky” gut physiology coupled with upregulation of the Toll-like receptor 4 (TLR4), a receptor well known for recognizing LPS and activating the innate immune system (Venkatesh et al., 2014 Symbiotic Bacterial Metabolites Regulate Gastrointestinal Barrier Function via the Xenobiotic Sensor PXR and Toll-like Receptor 4; Immunity 41, 296-310, Aug. 21, 2014). In particular, indole 3-propionic acid (IPA), produced by microbiota in the gut, has been shown to be a ligand for PXR in vivo.

As a result of PXR agonism, indole metabolite levels e.g., produced by commensal bacteria, or by genetically engineered bacteria, may through the activation of PXR regulate and balance the levels of TLR4 expression to promote homeostasis and gut barrier health. In other words, low levels of IPA and/or PXR and an excess of TLR4 may lead to intestinal barrier dysfunction, while increasing levels of IPA may promote PXR activation and TLR4 downregulation, and improved gut barrier health.

Although microbial degradation of tryptophan to indole-3-propionate has been shown in a numver of microorganisms (see, e.g., Elsden et al., The end products of the metabolism of aromatic amino acids by Clostridia, Arch Microbiol. 1976 Apr. 1; 107(3):283-8), to date, the bacterial entire biosynthetic pathway from tryptophan to IPA is unknown. In Clostridium sporogenes, tryptophan is catabolized via indole-3-pyruvate, indole-3-lactate, and indole-3-acrylate to indole-3-propionate (O'Neill and DeMoss, Tryptophan transaminase from Clostridium sporogenes, Arch Biochem Biophys. 1968 Sep. 20; 127(1):361-9). Two enzymes that have been purified from C. sporogenes are tryptophan transaminase and indole-3-lactate dehydrogenase (Jean and DeMoss, Indolelactate dehydrogenase from Clostridium sporogenes, Can J Microbiol. 1968 April; 14(4):429-35). Lactococcus lactis, catabolizes tryptophan by an aminotransferase to indole-3-pyruvate. In Lactobacillus casei and Lactobacillus helveticus tryptophan is also catabolized to indole-3-lactate through successive transamination and dehydrogenation (see, e.g., Tryptophan catabolism by Lactobacillus casei and Lactobacillus helveticus cheese flavor adjuncts Gummalla, S., Broadbent, J. R. J. Dairy Sci 82:2070-2077, and references therein).

L-tryptophan transaminase (e.g., EC 2.6.1.27, e.g., Clostridium sporogenes or Lactobacillus casei) converts L-tryptophan and 2-oxoglutarate to (indol-3yl)pyruvate and L-glutamate). Indole-3-lactate dehydrogenase (EC 1.1.1.110, e.g., Clostridium sporogenes or Lactobacillus casei) converts (indol-3yl) pyruvate and NADH and H+ to indole-3 lactate and NAD+.

In some embodiments, the engineered bacteria encode one or more enzymes selected from tryptophan transaminase (e.g., from C. sporogenes) and/or indole-3-lactate dehydrogenase (e.g., from C. sporogenes), and/or indole-3-pyruvate aminotransferase (e.g., from Lactococcus lactis). In other embodiments, such enzymes encoded by the bacteria are from Lactobacillus casei and/or Lactobacillus helveticus.

In other embodiments, IPA producing circuits comprise enzymes depicted and described in FIG. 6A and FIG. 6B and elsewhere herein.

D. Methoxyindole pathway, Serotonin and Melatonin

The methoxyindole pathway leads to formation of serotonin (5-HT) and melatonin. Serotonin (5-hydroxytryptamine, 5-HT) is a biogenic amine synthesized in a two-step enzymatic reaction: First, enzymes encoded by one of two tryptophan hydroxylase genes (Tph1 or Tph2) catalyze the rate-limiting conversion of tryptophan to 5-hydroxytryptophan (5-HTP). Tph1 2 is active in the brain and Tph2 is active in the periphery. Subsequently, 5-HTP undergoes decarboxylation to serotonin. Serotonin metabolism is independently regulated in the brain and periphery because the blood-brain barrier partitions bioavailability.

The majority (95%-98%) of total body serotonin is found in the gut (Berger et al., 2009). Peripheral serotonin acts autonomously on many cells, tissues, and organs, including the cardiovascular, gastrointestinal, hematopoietic, and immune systems as well as bone, liver, and placenta (Amireault et al., 2013). Serotonin functions as a ligand for any of 15 membrane-bound mostly G protein-coupled serotonin receptors (5-HTRs) that are involved in various signal transduction pathways in both CNS and periphery. Intestinal serotonin is released by enterochromaffin cells and neurons and is regulated via the serotonin re-uptake transporter (SERT). The SERT is located on epithelial cells and neurons in the intestine. Gut microbiota are interconnected with serotonin signaling and are for example capable of increasing serotonin levels through host serotonin production (Jano et al., Cell. 2015 Apr. 9; 161(2):264-76. doi: 10.1016/j.cell.2015.02.047. Indigenous bacteria from the gut microbiota regulate host serotonin biosynthesis).

Modulation of tryptophan metabolism, especially serotonin synthesis is considered a novel potential strategy the treatment of gastrointestinal (GI) disorders. For example, diarrhea-predominant irritable bowel disorder (IBD) is associated with elevated serotonin, while constipation-predominant IBD) is associated with decreased levels of serotonin in the colon mucosa.

In certain embodiments, the genetically engineered bacteria described herein may modulate serotonin levels in the gut, e.g., decrease or increase serotonin levels in the gut. In certain embodiments, the genetically engineered bacteria influence serotonin synthesis, release, and/or degradation. In some embodiments, the genetically engineered bacteria may modulate the serotonin levels in the gut to improve gut barrier function, modulate the inflammatory status or otherwise ameliorate symptoms of a metabolic disease and/or an gastrointestinal disorder or inflammatory bowel disorder. In some embodiments, the genetically engineered bacteria take up serotonin from the environment, e.g., the gut. In some embodiments, the genetically engineered bacteria release serotonin into the environment, e.g., the gut. In some embodiments, the genetically engineered modulate or influence serotonin levels produced by the host. In some embodiments, the genetically engineered bacteria counteract microbiota which are responsible for altered serotonin function in many GI diseases.

In some embodiments, the genetically engineered bacteria comprise tryptophan hydroxylase (TpH (1 and/or 2)) and/or 1-amino acid decarboxylase, e.g. for the treatment of constipation-associated IBD. In some embodiments, the genetically engineered bacteria comprise cassettes which allow trptophan uptake and catalysis, reducing trptophan availability for serotonin synthesis (serotonin depletion). In some embodiments, the genetically engineered bacteria comprise cassettes which promote serotonin uptake from the environment, e.g., the gut, and serotonin catalysis.

The Tph2-dependent serotoninergic system acts solely at specific sites in the brain, which accounts for 2%-5% of total body serotonin. In the brain, serotonin modulates mood, anxiety, appetite, and potentially cognitive performance. In certain embodiments, the genetically engineered bacteria described herein may modulate serotonin levels in the brain.

Additionally, serotonin also functions a substrate for melatonin biosynthesis. Melatonin acts as a neurohormone and is associated with the development of circadian rhythm and the sleep-wake cycle. Melatonin is a well-known lipophylic hormone produced at night by pineal gland and after feeding with tryptophan containing protein or tryptophan itself by neuroendocrine cells of the digestive system. It acts through high-affinity Gprotein-coupled membrane receptors through endocrine, paracrine or neurocrine pathway to protect the mucosa of the upper gastrointestinal tract from various irritants and ulcerogens.

The rate-limiting step of melatonin biosynthesis is 5-HT-N-acetylation resulting in the formation of N-acetyl-serotonin (NAS) with subsequent Omethylation into 5-methoxy-N-acetyltryptamine (melatonin). The deficient production of 5-HT, NAS, and melatonin contribute to depressed mood, disturbances of sleep and circadian rhythms.

In bacteria, melatonin is synthesized indirectly with tryptophan as an intermediate product of the shikimic acid pathway. In these cells, synthesis starts with d-erythrose-4-phosphate and phosphoenolpyruvate. In some embodiments the genetically engineered bacteria comprise an endogenous or exogenous cassette for the production of melatonin. As a non-limiting example, the cassette is described in Bochkov, Denis V.; Sysolyatin, Sergey V.; Kalashnikov, Alexander I.; Surmacheva, Irina A. (2011). “Shikimic acid: review of its analytical, isolation, and purification techniques from plant and microbial sources”. Journal of Chemical Biology 5 (1): 5-17. doi:10.1007/s12154-011-0064-8.

In a non-limiting example, genetically engineered bacteria convert tryptophan and/or serotonin to melatonin by, e.g., tryptophan hydroxylase (TPH), hydroxyl-O-methyltransferase (HIOMT), N-acetyltransferase (NAT), and aromatic—amino acid decarboxylase (AAAD), or equivalents thereof, e.g., bacterial equivalents.

II. Tryptophan and Tryptophan Metabolite Circuits

(a) Decreasing Exogenous Tryptophan

In some embodiments, the genetically engineered bacteria are capable of decreasing the level of tryptophan and/or the level of a tryptophan metabolite. In some embodiments, the engineered bacteria comprise gene sequence(s) for encoding one or more aromatic amino acid transporter(s). In one embodiment, the amino acid transporter is a tryptophan transporter. Tryptophan transporters may be expressed or modified in the recombinant bacteria described herein in order to enhance tryptophan transport into the cell. Specifically, when the tryptophan transporter is expressed in the recombinant bacterial cells described herein, the bacterial cells import more tryptophan into the cell when the transporter is expressed than unmodified bacteria of the same bacterial subtype under the same conditions. Thus, the genetically engineered bacteria comprising a heterologous gene encoding a tryptophan transporter which may be used to import tryptophan into the bacteria.

The uptake of tryptophan into bacterial cells is mediated by proteins well known to those of skill in the art. For example, three different tryptophan transporters, distinguishable on the basis of their affinity for tryptophan have been identified in E. coli (see, e.g., Yanofsky et al. (1991) J. Bacteriol. 173: 6009-17). The bacterial genes mtr, aroP, and tnaB encode tryptophan permeases responsible for tryptophan uptake in bacteria. High affinity permease, Mtr, is negatively regulated by the trp repressor and positively regulated by the TyR product (see, e.g., Yanofsky et al. (1991) J. Bacteriol. 173: 6009-17 and Heatwole et al. (1991) J. Bacteriol. 173: 3601-04), while AroP is negatively regulated by the tyR product (Chye et al. (1987) J. Bacteriol. 169:386-93).

In one embodiment, the at least one gene encoding a tryptophan transporter is a gene selected from the group consisting of mtr, aroP and tnaB. In one embodiment, the bacterial cell described herein has been genetically engineered to comprise at least one heterologous gene selected from the group consisting of mtr, aroP and tnaB. In one embodiment, the at least one gene encoding a tryptophan transporter is the Escherichia coli mtr gene. In one embodiment, the at least one gene encoding a tryptophan transporter is the Escherichia coli aroP gene. In one embodiment, the at least one gene encoding a tryptophan transporter is the Escherichia coli tnaB gene.

In some embodiments, the tryptophan transporter is encoded by a tryptophan transporter gene derived from a bacterial genus or species, including but not limited to, Escherichia, Corynebacterium, Escherichia coli, Saccharomyces cerevisiae or Corynebacterium glutamicum. In some embodiments, the bacterial species is Escherichia coli. In some embodiments, the bacterial species is Escherichia coli strain Nissle.

Assays for testing the activity of a tryptophan transporter, a functional variant of a tryptophan transporter, or a functional fragment of transporter of tryptophan are well known to one of ordinary skill in the art. For example, import of tryptophan may be determined using the methods as described in Shang et al. (2013) J. Bacteriol. 195:5334-42, the entire contents of each of which are expressly incorporated by reference herein.

In one embodiment, when the tryptophan transporter is expressed in the recombinant bacterial cells described herein, the bacterial cells import 10% more tryptophan into the bacterial cell when the transporter is expressed than unmodified bacteria of the same bacterial subtype under the same conditions. In another embodiment, when the tryptophan transporter is expressed in the recombinant bacterial cells described herein, the bacterial cells import 20%, 30%, 40%, 50%, 60%, 70%, 80%, 90% or 100% more tryptophan into the bacterial cell when the transporter is expressed than unmodified bacteria of the same bacterial subtype under the same conditions. In yet another embodiment, when the tryptophan transporter is expressed in the recombinant bacterial cells described herein, the bacterial cells import two-fold more tryptophan into the cell when the transporter is expressed than unmodified bacteria of the same bacterial subtype under the same conditions. In yet another embodiment, when the tryptophan transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, fourty-fold, or fifty-fold, more tryptophan into the cell when the transporter is expressed than unmodified bacteria of the same bacterial subtype under the same conditions.

In some embodiments, the genetically engineered bacteria are capable of decreasing the level of tryptophan. In some embodiments, the engineered bacteria comprises one or more gene sequences for converting tryptophan to kynurenine. In some embodiments, the engineered bacteria comprises gene sequence(s) for encoding the enzyme indoleamine 2,3-dioxygenase (IDO-1). In some embodiments, the engineered bacteria comprises gene sequence(s) for encoding the enzyme tryptophan dioxygenase (TDO). In some embodiments, the engineered bacteria comprises gene sequence(s) for encoding the enzyme indoleamine 2,3-dioxygenase (IDO-1) and the enzyme tryptophan dioxygenase (TDO). In some embodiments, the genetically engineered bacteria comprise a gene cassette encoding Indoleamine 2, 3 dioxygenase (EC 1.13.11.52; producing N-formyl kynurenine from tryptophan) and Kynurenine formamidase (EC3.5.1.9) producing kynurenine from n-formylkynurenine). In some embodiments, the enzymes are bacterially derived, e.g., as described in Vujkovi-Cvijin et al. 2013.

In some embodiments, the genetically engineered bacteria are capable of decreasing the level of tryptophan, e.g., in combination with the production of indole metabolites, through expression of gene(s) and gene cassette(s) described herein.

(b) Increasing Kynurenine

In some embodiments, the genetically engineered bacteria are capable of producing kynurenine.

In some embodiments, the genetically engineered bacteria are capable of decreasing the level of tryptophan. In some embodiments, the engineered bacteria comprises one or more gene sequences for converting tryptophan to kynurenine. In some embodiments, the engineered bacteria comprises gene sequence(s) for encoding the enzyme indoleamine 2,3-dioxygenase (IDO-1). In some embodiments, the engineered bacteria comprises gene sequence(s) for encoding the enzyme tryptophan dioxygenase (TDO). In some embodiments, the engineered bacteria comprise on or more gene sequence(s) for encoding the enzyme indoleamine 2,3-dioxygenase (IDO-1) and the enzyme tryptophan dioxygenase (TDO). In some embodiments, the genetically engineered bacteria comprise a gene cassette encoding Indoleamine 2, 3 dioxygenase (EC 1.13.11.52; producing N-formyl kynurenine from tryptophan) and Kynurenine formamidase (EC3.5.1.9) producing kynurenine from n-formylkynurenine). In some embodiments, the enzymes are bacterially derived, e.g., as described in Vujkovi-Cvijin et al. 2013.

In one embodiment, the genetically engineered bacteria comprise one or more gene sequence(s) which encode one or more tryptophan catabolism enzymes, which produce kynurenine from tryptophan. Non-limiting example of such gene sequence(s) and described elsewhere herein. In one embodiment, the genetically engineered bacteria comprise one or more gene sequence(s) which encode IDO1 (indoleamine 2,3-dioxygenase). In one embodiment, the genetically engineered bacteria comprise one or more gene sequence(s) which encode IDO1 from Homo sapiens. In one embodiment, the genetically engineered bacteria comprise one or more gene sequence(s) which encode TDO2 (tryptophan 2,3-dioxygenase). In one embodiment, the genetically engineered bacteria comprise one or more gene sequence(s) which encode TDO2 from Homo sapiens. In one embodiment, the genetically engineered bacteria comprise one or more gene sequence(s) which encode BNA2 (indoleamine 2,3-dioxygenase). In one embodiment, the genetically engineered bacteria comprise one or more gene sequence(s) which encode BNA2 from S. cerevisiae). In one embodiment, the genetically engineered bacteria comprise one or more gene sequence(s) which encode Afmid: Kynurenine formamidase. In one embodiment, the genetically engineered bacteria comprise one or more gene sequence(s) which encode Afmid: Kynurenine formamidase from mouse. In one embodiment, the genetically engineered bacteria comprise one or more gene sequence(s) which encode Afmid in combination with one or more of idol and/or tdo2 and/or bna2. In one embodiment, the genetically engineered bacteria comprise one or more gene sequence(s) which encode Afmid in combination with idol. In one embodiment, the genetically engineered bacteria comprise one or more gene sequence(s) which encode BNA2 in combination with tdo2. In one embodiment, the genetically engineered bacteria comprise one or more gene sequence(s) which encode Afmid in combination with bna2. In one embodiment, the genetically engineered bacteria comprise one or more gene sequence(s) which encode BNA3 (kynurenine—oxoglutarate transaminase. In one embodiment, the genetically engineered bacteria comprise one or more gene sequence(s) which encode BNA3 from S. cerevisae. In one embodiment, the genetically engineered bacteria comprise one or more gene sequence(s) which encode BNA2 in combination with one or more of idol and/or tdo2 and/or bna2. In one embodiment, the genetically engineered bacteria comprise one or more gene sequence(s) which encode BNA2 in combination with idol. In one embodiment, the genetically engineered bacteria comprise one or more gene sequence(s) which encode BNA2 in combination with tdo2. In one embodiment, the genetically engineered bacteria comprise one or more gene sequence(s) which encode BNA2 in combination with bna2. In one embodiment, the genetically engineered bacteria comprise one or more gene sequence(s) which encode one or more of idol and/or tdo2 and/or bna2. In one embodiment, the genetically engineered bacteria comprise one or more gene sequence(s) which encode one or more of afmid and/or bna3.

In one embodiment, the genetically engineered bacteria comprise one or more gene sequence(s) which encode one or more of idol and/or tdo2 and/or bna2, in combination with one or more of afmid and/or bna3.

In any of these embodiments, the genetically engineered bacteria which produce kynurenine from tryptophan also optionally comprise one or more gene sequence(s) comprising one or more enzymes for tryptophan production, and gene deletions/or mutations as depicted and described described elsewhere herein. In some embodiments, the genetically engineered bacteria which produce kynurenine from tryptophan also optionally comprise one or more gene sequence(s) which encode one or more transporter(s) as described herein, through which tryptophan can be imported. Optionally, in some embodiments, the genetically engineered bacteria which produce kynurenine from tryptophan also optionally comprise one or more gene sequence(s) which encode an exporter as described herein, which can export tryptophan or any of its metabolites.

The genetically engineered bacteria may comprise any suitable gene for producing kynurenine. In some embodiments, the gene for producing kynurenine is modified and/or mutated, e.g., to enhance stability, increase kynurenine production, and/or increase anti-inflammatory potency under inducing conditions. In some embodiments, the engineered bacteria also have enhanced uptake or import of tryptophan, e.g., comprise a transporter or other mechanism for increasing the uptake of tryptophan into the bacterial cell, as discussed in detail above. In some embodiments, the genetically engineered bacteria are capable of producing kynurenine under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with disorders, such as liver damage, metabolic disease, inflammation or an inflammatory response, or in the presence of some other metabolite that may or may not be present in the gut, such as arabinose.

In some embodiments, the genetically engineered bacteria comprise one or more gene(s) or gene cassette(s) for the consumption of tryptophan and production of kynurenine, which are bacterially derived. In some embodiments, the enzymes for TRP to KYN conversion are derived from one or more of Pseudomonas, Xanthomonas, Burkholderia, Stenotrophomonas, Shewanella, and Bacillus, and/or members of the families Rhodobacteraceae, Micrococcaceae, and Halomonadaceae. In some embodiments the enzymes are derived from the species listed in table S7 of Vujkovic-Cvijin et al. (Dysbiosis of the gut microbiota is associated with HIV diseaseprogression and tryptophan catabolism Sci Transl Med. 2013 Jul. 10; 5(193): 193ra91), the contents of which is herein incorporated by reference in its entirety.

In some embodiments, the one or more genes for producing kynurenine are modified and/or mutated, e.g., to enhance stability, increase kynurenine production, and/or increase anti-inflammatory potency under inducing conditions. In some embodiments, the engineered bacteria have enhanced uptake or import of tryptophan, e.g., comprise a transporter or other mechanism for increasing the uptake of tryptophan into the bacterial cell. In some embodiments, the genetically engineered bacteria are capable of producing kynurenine under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

In some embodiments, the genetically engineered bacteria are capable of expressing any one or more of the described circuits in low-oxygen conditions, in the presence of disease or tissue specific molecules or metabolites, in the presence of molecules or metabolites associated with inflammation or an inflammatory response or immune suppression, liver damage, or metabolic disease, or in the presence of some other metabolite that may or may not be present in the gutor the tumor microenvironment, such as arabinose. In some embodiments, any one or more of the described circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits.

(c) Increasing Tryptophan

In some embodiments, the genetically engineered microorganisms of the present disclosure are capable of producing tryptophan

In some embodiments, the genetically engineered bacteria that produce tryptophan comprise one or more gene sequences encoding one or more enzymes of the tryptophan biosynthetic pathway. In some embodiments, the genetically engineered bacteria comprise a tryptophan operon. In some embodiments, the genetically engineered bacteria comprise the tryptophan operon of E. coli. (Yanofsky, RNA (2007), 13:1141-1154). In some embodiments, the genetically engineered bacteria comprise the tryptophan operon of B. subtilis. (Yanofsky, RNA (2007), 13:1141-1154). In some embodiments, the genetically engineered bacteria comprise sequence(s) encoding trypE, trypG-D, trypC-F, trypB, and trpA genes. In some embodiments, the genetically engineered bacteria comprise sequence(s) encoding trypE, trypG-D, trypC-F, trypB, and trpA genes from E. Coli. In some embodiments, the genetically engineered bacteria comprise sequence(s) encoding trypE, trypD, trypC, trypF, trypB, and trpA genes from B. subtilis.

Also, in any of these embodiments, the genetically engineered bacteria optionally comprise gene sequence(s) to produce the tryptophan precursor, chorismate. Thus, in some embodiments, the genetically engineered bacteria optionally comprise sequence(s) encoding aroG, aroF, aroH, aroB, aroD, aroE, aroK, and AroC. In some embodiments, the genetically engineered bacteria comprise one or more gene sequences encoding one or more enzymes of the tryptophan biosynthetic pathway and one or more gene sequences encoding one or more enzymes of the chorismate biosynthetic pathway. In some embodiments, the genetically engineered bacteria comprise sequence(s) encoding trypE, trypG-D, trypC-F, trypB, and trpA genes from E. Coli and sequence(s) encoding aroG, aroF, aroH, aroB, aroD, aroE, aroK, and AroC genes. In some embodiments, the genetically engineered bacteria comprise sequence(s) encoding trypE, trypD, trypC, trypF, trypB, and trpA genes from B. subtilis and sequence(s) encoding aroG, aroF, aroH, aroB, aroD, aroE, aroK, and AroC genes.

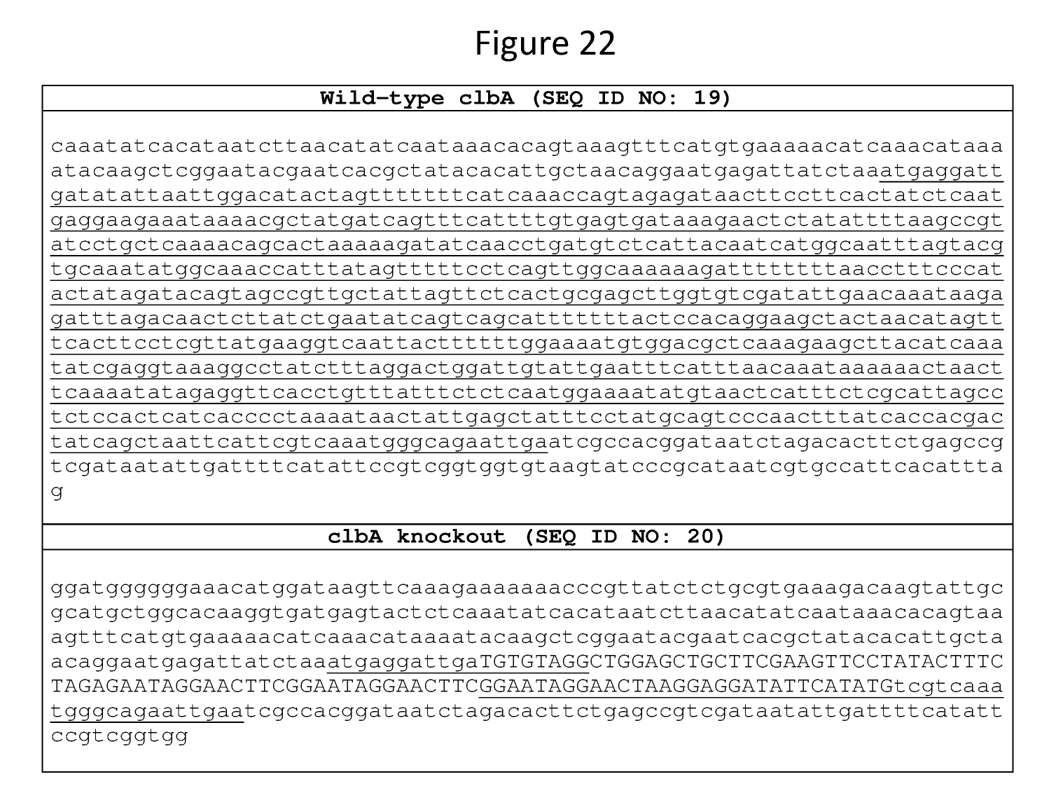

In some embodiments, the genetically engineered bacteria comprise sequence(s) encoding either a wild type or a feedback resistant SerA gene (Table 10). Escherichia coli serA-encoded 3-phosphoglycerate (3PG) dehydrogenase catalyzes the first step of the major phosphorylated pathway of L-serine (Ser) biosynthesis. This step is an oxidation of 3PG to 3-phosphohydroxypyruvate (3PHP) with the concomitant reduction of NAD+ to NADH. As part of Tryptophan biosynthesis, E. coli uses one serine for each tryptophan produced. As a result, by expressing serA, tryptophan production is improved (see, e.g., FIG. 22D.

In any of these embodiments, AroG and TrpE are optionally replaced with feedback resistant versions to improve tryptophan production (Table 10).

In any of these embodiments, the tryptophan repressor (trpR) optionally may be deleted, mutated, or modified so as to diminish or obliterate its repressor function.

In any of these embodiments the tnaA gene (encoding a tryptophanase converting Trp into indole) optionally may be deleted to prevent tryptophan catabolism along this pathway and to further increase levels of tryptophan produced (Table 10).

The inner membrane protein YddG of Escherichia coli, encoded by the yddG gene, is a homologue of the known amino acid exporters RhtA and YdeD. Studies have shown that YddG is capable of exporting aromatic amino acids, including tryptophan. Thus, YddG can function as a tryptophan exporter or a tryptophan secretion system (or tryptophan secretion protein). Other aromatic amino acid exporters are described in Doroshenko et al., FEMS Microbial Lett., 275:312-318 (2007). Thus, in some embodiments, the engineered bacteria optionally further comprise gene sequence(s) encoding YddG. In some embodiments, the engineered bacteria can over-express YddG. In some embodiments, the engineered bacteria optionally comprise one or more copies of yddG gene.

In some embodiments, the genetically engineered bacteria comprise a mechanism for metabolizing or degrading kynurenine, which, in some embodiments, also results in the increased production of tryptophan. In some embodiments, the genetically engineered bacteria modulate the TRP:KYN ratio or the KYN:TRP ratio in the extracellular environment. In some embodiments, the genetically engineered bacteria increase the TRP:KYN ratio or the KYN:TRP ratio. In some embodiments, the genetically engineered bacteria reduce the TRP:KYN ratio or the KYN:TRP ratio. In some embodiments, the genetically engineered bacteria comprise sequence encoding the enzyme kynureninase Kynureninase is produced to metabolize Kynurenine to Anthranilic acid in the cell. Schwarcz et al., Nature Reviews Neuroscience, 13, 465-477; 2012; Chen & Guillemin, 2009; 2; 1-19; Intl. J. Tryptophan Res. Exemplary kynureninase sequences are provided herein below in Table 11. In some embodiments, the engineered microbe has a mechanism for importing (transporting) kynurenine from the local environment into the cell. In some embodiments, the genetically engineered bacteria comprise one or more copies of aroP, tnaB or mtr gene. In some embodiments, the genetically engineered bacteria comprise gene sequence(s) encoding a kynureninase secreter.

In some embodiments, the genetically engineered bacteria comprise gene sequence(s) encoding enzymes of the tryptophan biosynthetic pathway and sequence encoding kynureninase. In some embodiments, the genetically engineered bacteria comprise a tryptophan operon, for example that of E. coli or B. subtilis, and sequence encoding kynureninase. In some embodiments, the genetically engineered bacteria comprise sequence(s) encoding trypE, trypG-D, trypC-F, trypB, and trpA genes, for example, from E. Coli and sequence encoding kyureninase. In some embodiments, the genetically engineered bacteria comprise sequence(s) encoding trypE, trypD, trypC, trypF, trypB, and trpA genes, for example from B. subtilis and sequence encoding kyureninase. In any of these embodiments, the tryptophan repressor (trpR) optionally may be deleted, mutated, or modified so as to diminish or obliterate its repressor function. Also, in any of these embodiments, the genetically engineered bacteria optionally comprise gene sequence(s) to produce the tryptophan precursor, Chorismate, for example, sequence(s) encoding aroG, aroF, aroH, aroB, aroD, aroE, aroK, and AroC. Thus, in some embodiments, the genetically engineered bacteria comprise sequence(s) encoding trypE, trypG-D, trypC-F, trypB, and trpA genes from E. Coli, sequence(s) encoding aroG, aroF, aroH, aroB, aroD, aroE, aroK, and AroC genes, and sequence encoding kyureninase. In some embodiments, the genetically engineered bacteria comprise sequence(s) encoding trypE, trypD, trypC, trypF, trypB, and trpA genes from B. subtilis, sequence(s) encoding aroG, aroF, aroH, aroB, aroD, aroE, aroK, and AroC genes, and sequence encoding kyureninase.

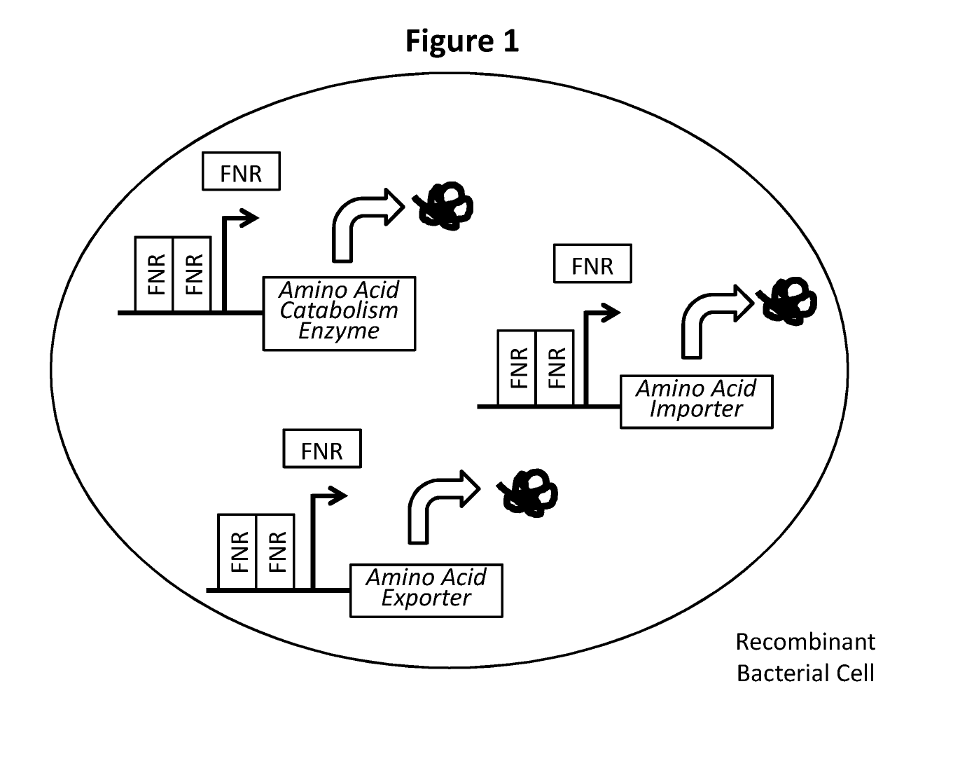

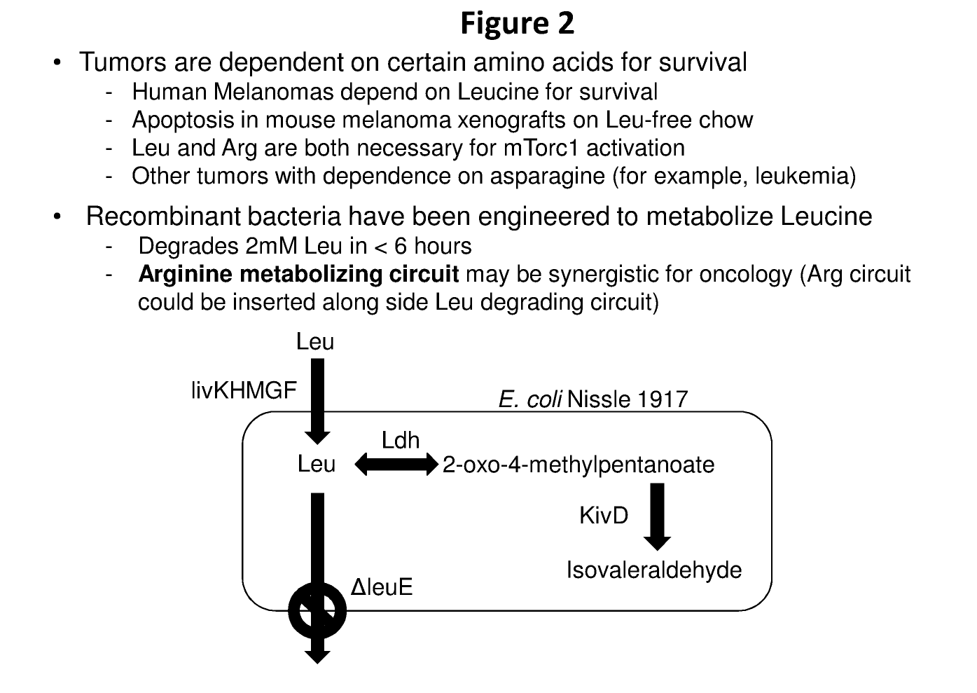

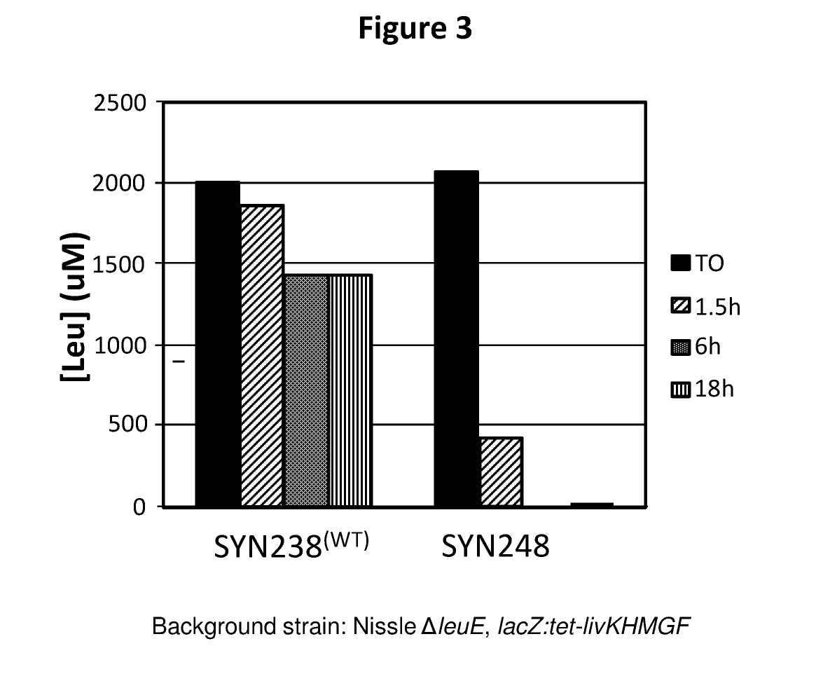

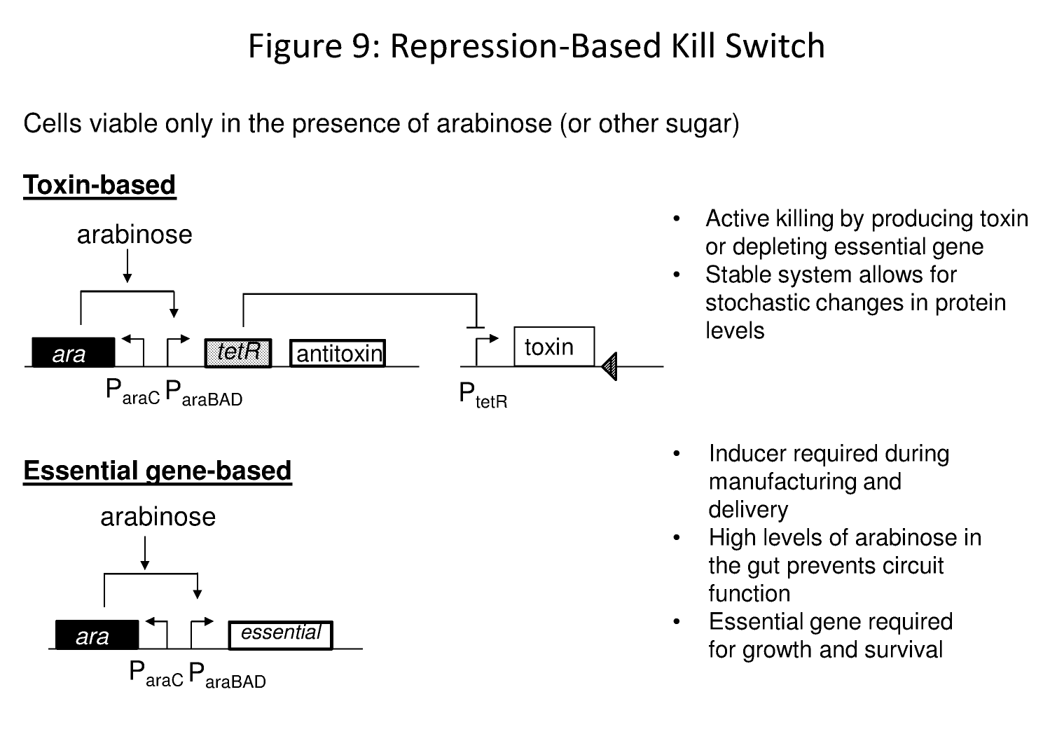

In some embodiments, the genetically engineered bacteria may optionally have a deletion or mutation in the endogenous trpE, rendering trpE non-functional. Accordingly, in one embodiment, the genetically engineered bacteria may comprise one or more gene(s) or gene cassette(s) encoding trpD, trpC, trpA, and trpD and kynureninase (see, e.g. FIG. 1 and FIG. 9). This deletion may prevent tryptophan production through the endogenous chorismate pathway, and may increase the production of tryptophan from kynurenine through kynureninase.

In some embodiments, the genetically engineered bacteria comprise sequence(s) encoding either a wild type or a feedback resistant SerA gene (Table 10).

In any of these embodiments, AroG and TrpE are optionally replaced with feedback resistant versions to improve tryptophan production (Table 10).

In any of these embodiments, the tryptophan repressor (trpR) optionally may be deleted, mutated, or modified so as to diminish or obliterate its repressor function.

In any of these embodiments the tnaA gene (encoding a tryptophanase converting Trp into indole) optionally may be deleted to prevent tryptophan catabolism along this pathway and to further increase levels of tryptophan produced (Table 10).

In any of these embodiments, the genetically engineered bacterium may further comprise gene sequence for exporting or secreting tryptophan from the cell. Thus, in some embodiments, the engineered bacteria further comprise gene sequence(s) encoding YddG. In some embodiments, the engineered bacteria can over-express YddG, an aromatic amino acid exporter. In some embodiments, the engineered bacteria optionally comprise one or more copies of yddG gene. In any of these embodiments, the genetically engineered bacterium may further comprise gene sequence for importing or transporting kynurenine into the cell. Thus, in some embodiments, the genetically engineered bacteria comprise gene sequence(s) encoding a kynureninase secreter. In some embodiments, the genetically engineered bacteria comprise one or more copies of aroP, tnaB or mtr gene.

In some embodiments, the genetically engineered bacterium or genetically engineered microorganism comprises one or more genes for producing tryptophan and/or kynureninase, under the control of a promoter that is activated by low-oxygen conditions, by inflammatory conditions, liver damage, and.or metabolic disease, such as any of the promoters activated by said conditions and described herein. In some embodiments, the genetically engineered bacteria expresses one or more genes for producing tryptophan and/or kynureninase, under the control of a cancer-specific promoter, a tissue-specific promoter, or a constitutive promoter, such as any of the promoters described herein. The genetically engineered bacteria may comprise any suitable gene for producing kynureninase. In some embodiments, the gene for producing kynureninase is modified and/or mutated, e.g., to enhance stability, increase kynureninase production. In some embodiments, the engineered bacteria also have enhanced uptake or import of tryptophan, e.g., comprise a transporter or other mechanism for increasing the uptake of tryptophan into the bacterial cell, as discussed in detail above. In some embodiments, the genetically engineered bacteria are capable of producing kynureninase under inducing conditions, e.g., under a condition(s) associated with inflammation. In some embodiments, the genetically engineered bacteria are capable of producing kynureninase in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

The genetically engineered bacteria may comprise any suitable gene for producing kynureninase. In some embodiments, the gene for producing kynureninase is modified and/or mutated, e.g., to enhance stability, increase kynureninase production. In some embodiments, the engineered bacteria also have enhanced uptake or import of tryptophan, e.g., comprise a transporter or other mechanism for increasing the uptake of tryptophan into the bacterial cell, as discussed in detail above. In some embodiments, the genetically engineered bacteria are capable of producing kynureninase under inducing conditions, e.g., under a condition(s) associated with inflammation. In some embodiments, the genetically engineered bacteria are capable of producing kynureninase in low-oxygen conditions. In some embodiments, the genetically engineered bacteria are capable of producing kynureninase in the presence of certain molecules or metabolites, in the presence of molecules or metabolites associated with liver damage, metabolic disease, inflammation or an inflammatory response, or in the presence of some other metabolite that may or may not be present in the gut, such as arabinose.

In some embodiments, the genetically engineered bacteria are capable of expressing any one or more of the described circuits in low-oxygen conditions, in the presence of disease or tissue specific molecules or metabolites, in the presence of molecules or metabolites associated with inflammation or an inflammatory response or immune suppression, liver damage, metabolic disease, or in the presence of some other metabolite that may or may not be present in the gut, such as arabinose. In some embodiments, any one or more of the described circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits.

(d) Producing Kynurenic Acid

In some embodiments, the genetically engineered bacteria are capable of producing kynurenic acid. Kynurenic acid is produced from the irreversible transamination of kynurenine in a reaction catalyzed by the enzyme kynurenine-oxoglutarate transaminase.

In some embodiments, the gene or genes for producing kynurenic acid is modified and/or mutated, e.g., to enhance stability, increase kynurenic acid production under inducing conditions. In some embodiments, the genetically engineered bacteria are capable of producing kynurenic acid under inducing conditions, e.g., under a condition(s) associated with inflammation. In some embodiments, the genetically engineered bacteria are capable of producing kynurenic acid in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

In some embodiments, the genetically engineered bacteria comprise one or more gene(s) or gene cassette(s) for the consumption of tryptophan and production of kynurenic acid, which are bacterially derived. In some embodiments, the enzymes for producing kynureic acid are derived from one or more of Pseudomonas, Xanthomonas, Burkholderia, Stenotrophomonas, Shewanella, and Bacillus, and/or members of the families Rhodobacteraceae, Micrococcaceae, and Halomonadaceae. In some embodiments the enzymes are derived from the species listed in table S7 of Vujkovic-Cvijin et al. (Dysbiosis of the gut microbiota is associated with HIV diseaseprogression and tryptophan catabolism Sci Transl Med. 2013 Jul. 10; 5(193): 193ra91), the contents of which is herein incorporated by reference in its entirety.

In some embodiments, the genetically engineered bacteria comprise gene sequence(s) encoding one or more tryptophan transporters and gene sequence(s) encoding kynureninase. In some embodiments, the genetically engineered bacteria comprise gene sequence(s) encoding one or more tryptophan transporters and gene sequence(s) encoding one or more kynurenine-oxoglutarate transaminases (kynurenine aminotransferases). In some embodiments, the genetically engineered bacteria comprise gene sequence(s) encoding one or more tryptophan transporters, gene sequence(s) encoding kynureninase, and gene sequence(s) encoding one or more kynurenine-oxoglutarate transaminases (kynurenine aminotransferases). In some embodiments, the genetically engineered bacteria comprise gene sequence(s) encoding kynureninase and gene sequence(s) encoding one or more kynurenine aminotransferases.

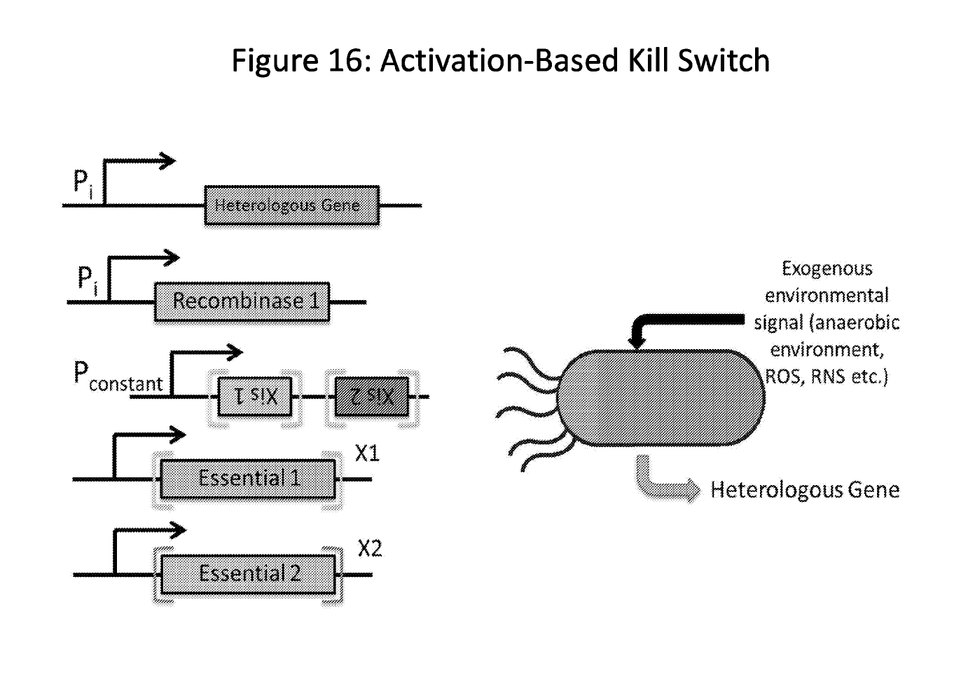

In one embodiment, the genetically engineered bacteria comprise one or more gene sequence(s) which encode one or more tryptophan catabolism enzymes, which produce kynurenic acid from tryptophan. Non-limiting example of such gene sequence(s) are shown FIG. 16F and described elsewhere herein. In one embodiment, the genetically engineered bacteria comprise one or more gene sequence(s) which encode IDO1 (indoleamine 2,3-dioxygenase). In one embodiment, the genetically engineered bacteria comprise one or more gene sequence(s) which encode IDO1 from Homo sapiens. In one embodiment, the genetically engineered bacteria comprise one or more gene sequence(s) which encode TDO2 (tryptophan 2,3-dioxygenase). In one embodiment, the genetically engineered bacteria comprise one or more gene sequence(s) which encode TDO2 from Homo sapiens. In one embodiment, the genetically engineered bacteria comprise one or more gene sequence(s) which encode BNA2 (indoleamine 2,3-dioxygenase). In one embodiment, the genetically engineered bacteria comprise one or more gene sequence(s) which encode BNA2 from S. cerevisiae). In one embodiment, the genetically engineered bacteria comprise one or more gene sequence(s) which encode Afmid: Kynurenine formamidase. In one embodiment, the genetically engineered bacteria comprise one or more gene sequence(s) which encode Afmid: Kynurenine formamidase from mouse. In one embodiment, the genetically engineered bacteria comprise one or more gene sequence(s) which encode Afmid in combination with one or more of idol and/or tdo2 and/or bna2. In one embodiment, the genetically engineered bacteria comprise one or more gene sequence(s) which encode Afmid in combination with IDO1. In one embodiment, the genetically engineered bacteria comprise one or more gene sequence(s) which encode BNA2 in combination with TDO2. In one embodiment, the genetically engineered bacteria comprise one or more gene sequence(s) which encode Afmid in combination with bna2. In one embodiment, the genetically engineered bacteria further comprise one or more gene sequence(s) which encode cclb1 and/or cclb2 and/or aadat and/or got2. In one embodiment, the genetically engineered bacteria comprise one or more gene sequence(s) which encode BNA3 (kynurenine—oxoglutarate transaminase. In one embodiment, the genetically engineered bacteria comprise one or more gene sequence(s) which encode BNA3 from S. cerevisae. In one embodiment, the genetically engineered bacteria comprise one or more gene sequence(s) which encode BNA2 in combination with one or more of idol and/or tdo2 and/or bna2. In one embodiment, the genetically engineered bacteria comprise one or more gene sequence(s) which encode BNA2 in combination with idol. In one embodiment, the genetically engineered bacteria comprise one or more gene sequence(s) which encode BNA2 in combination with tdo2. In one embodiment, the genetically engineered bacteria comprise one or more gene sequence(s) which encode BNA2 in combination with bna2. In one embodiment, the genetically engineered bacteria further comprise one or more gene sequence(s) which encode cclb1 and/or cclb2 and/or aadat and/or got2. In one embodiment, the genetically engineered bacteria comprise one or more gene sequence(s) which encode one or more of idol and/or tdo2 and/or bna2.

In one embodiment, the genetically engineered bacteria comprise one or more gene sequence(s) which encode one or more of afmid and/or bna3. In one embodiment, the genetically engineered bacteria comprise one or more gene sequence(s) which encode one or more of idol and/or tdo2 and/or bna2, in combination with one or more of afmid and/or bna3. In one embodiment, the genetically engineered bacteria comprise one or more gene sequence(s) which encode GOT2 (Aspartate aminotransferase, mitochondrial). In one embodiment, the genetically engineered bacteria comprise one or more gene sequence(s) which encode GOT2 from Homo sapiens. In one embodiment, the genetically engineered bacteria comprise one or more gene sequence(s) which encode AADAT (Kynurenine/alpha-aminoadipate aminotransferase, mitochondrial). In one embodiment, the genetically engineered bacteria comprise one or more gene sequence(s) which encode AADAT from Homo sapiens. In one embodiment, the genetically engineered bacteria comprise one or more gene sequence(s) which encode CCLB1 (Kynurenine—oxoglutarate transaminase). In one embodiment, the genetically engineered bacteria comprise one or more gene sequence(s) which encode CCLB1 from Homo sapiens). In one embodiment, the genetically engineered bacteria comprise one or more gene sequence(s) which encode CCLB2 (kynurenine—oxoglutarate transaminase 3) In one embodiment, the genetically engineered bacteria comprise one or more gene sequence(s) which encode CCLB2 from Homo sapiens. In one embodiment, the genetically engineered bacteria comprise one or more gene sequence(s) which encode cclb1 and/or cclb2 and/or aadat and/or got2. In one embodiment, the genetically engineered bacteria comprise one or more gene sequence(s) which encode one or more of idol and/or tdo2 and/or bna2, in combination with one or more of afmid and/or bna3, and in combination with one or more of .cclb1 and/or cclb2 and/or aadat and/or got2.

In any of these embodiments, the genetically engineered bacteria which produce kynurenic acid from tryptophan also optionally comprise one or more gene sequence(s) comprising one or more enzymes for tryptophan production, and gene deletions/or mutations as depicted and described elsewhere herein. In some embodiments, the genetically engineered bacteria which produce kynurenic acid from tryptophan also optionally comprise one or more gene sequence(s) which encode one or more transporter(s) as described herein, through which tryptophan can be imported. Optionally, in some embodiments, the genetically engineered bacteria which produce kynurenic acid from tryptophan also optionally comprise one or more gene sequence(s) which encode an exporter as described herein, which can export tryptophan or any of its metabolites.

In some embodiments, the one or more genes for producing kynurenic acid are modified and/or mutated, e.g., to enhance stability, increase kynurenic acid production under inducing conditions. In some embodiments, the engineered bacteria have enhanced uptake or import of tryptophan, e.g., comprise a transporter or other mechanism for increasing the uptake of tryptophan into the bacterial cell. In some embodiments, the genetically engineered bacteria are capable of producing kynurenic acid under inducing conditions, e.g., under a condition(s) associated with inflammation. In some embodiments, the genetically engineered bacteria are capable of producing kynurenic acid in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

In some embodiments, the genetically engineered bacteria are capable of expressing any one or more of the described circuits in low-oxygen conditions, in the presence of disease or tissue specific molecules or metabolites, in the presence of molecules or metabolites associated with inflammation or an inflammatory response or immune suppression, liver damage, metabolic disease, or in the presence of some other metabolite that may or may not be present in the gut, such as arabinose. In some embodiments, any one or more of the described circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits.

(e) Producing Indole Tryptophan Metabolites and Tryptamine

In some embodiments, the genetically engineered bacteria comprise genetic circuits for the production of indole metabolites and/or tryptamine

In in any of these embodiments the expression of the gene sequences for the production of the indole and other tryptophan metabolites, including, but not limited to, tryptamine and/or indole-3 acetaladehyde, indole-3acetonitrile, indole, indole acetic acid FICZ, indole-3-propionic acid is under the control of an inducible promoter. Exemplary inducible promoters which may control the expression of the biosynthetic cassettes include oxygen level-dependent promoters (e.g., FNR-inducible promoter), promoters induced by inflammation or an inflammatory response (RNS, ROS promoters), and promoters induced by a metabolite characteristic of a disorder, such as liver damage or a metabolic disease, or that may or may not be naturally present (e.g., can be exogenously added) in the gut, e.g., arabinose and tetracycline. In some embodiments, the one or more gene sequences(s) are under the control of a constitutive promoter.

In some embodiments, the genetically engineered bacteria are capable of expressing any one or more of the described circuits in low-oxygen conditions, in the presence of disease or tissue specific molecules or metabolites, in the presence of molecules or metabolites associated with inflammation or an inflammatory response or immune suppression, liver damage, or metabolic disease, or in the presence of some other metabolite that may or may not be present in the gutor the tumor microenvironment, such as arabinose. In some embodiments, any one or more of the described circuits are present on one or more plasmids (e.g., high copy or low copy) or are integrated into one or more sites in the bacterial chromosome. Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, and (6) combinations of one or more of such additional circuits.

(f) Tryptamine

In some embodiments the genetically engineered bacteria comprise one or more gene sequence(s) which encode one or more tryptophan catabolism enzymes, produce tryptamine from tryptophan. The monoamine alkaloid, tryptamine, is derived from the direct decarboxylation of tryptophan. Tryptophan is converted to indole-3-acetic acid (IAA) via the enzymes tryptophan monooxygenase (IaaM) and indole-3-acetamide hydrolase (IaaH), which constitute the indole-3-acetamide (IAM) pathway. In one embodiment, the genetically engineered bacteria comprise one or more gene sequence(s) which encode one or more Tryptophan decarboxylase(s). e.g., from Catharanthus roseus. In one embodiment the genetically engineered bacteria comprise one or more gene sequence(s) which encode one or more Tryptophan decarboxylase(s). e.g., from Catharanthus roseus. In one embodiment the genetically engineered bacteria comprise one or more gene sequence(s) which encode one or more Tryptophan decarboxylase(s). e.g., from Clostridium sporogenes. In one embodiment the genetically engineered bacteria comprise one or more gene sequence(s) which encode one or more Tryptophan decarboxylase(s) e.g., from Ruminococcus Gnavus.

In one embodiment, the genetically engineered bacteria comprise one or more gene sequence(s) which encode tdc(tryptophan decarboxylase). In one embodiment, the genetically engineered bacteria comprise one or more gene sequence(s) which encode tdc from Catharanthus roseus. In one embodiment, the genetically engineered bacteria comprise one or more gene sequence(s) which encode tdc from Clostridium sporogenes.

In some embodiments, the genetically engineered bacteria which produce tryptamine from tryptophan also optionally comprise one or more gene sequence(s) comprising one or more enzymes for tryptophan production, and gene deletions/or mutations as depicted and described elsewhere herein. In some embodiments, the genetically engineered bacteria which produce tryptamine from tryptophan also optionally comprise one or more gene sequence(s) which encode one or more transporter(s) as described herein, through which tryptophan can be imported. Optionally. In some embodiments, the genetically engineered bacteria which produce tryptamine from tryptophan also optionally comprise one or more gene sequence(s) which encode an exporter as described herein, which can export tryptophan or any of its metabolites.

In some embodiments, the genetically engineered bacteria are capable of producing Tryptamine under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

(g) Indole-3-acetaldehyde and FICZ

In one embodiment, the genetically engineered bacteria comprise one or more gene sequence(s) which encode one or more tryptophan catabolism enzymes, which produce indole-3-acetaldehyde and FICZ from tryptophan. Exemplary gene cassettes for the production of produce indole-3-acetaldehyde and FICZ from tryptophan are shown.

In one embodiment, the genetically engineered bacteria comprise one or more gene sequence(s) which encode aro9 (L-tryptophan aminotransferase). In one embodiment, the (L-tryptophan aminotransferase is from S. cerevisiae. In one embodiment, the genetically engineered bacteria comprise one or more gene sequence(s) which encode ipdC (Indole-3-pyruvate decarboxylase, e.g., from Enterobacter cloacae). In one embodiment, the genetically engineered bacteria comprise one or more gene sequence(s) which encode aro9 and ipdC. In one embodiment, the genetically engineered bacteria comprise one or more gene sequence(s) which encode aspC (aspartate aminotransferase. In one embodiment, the genetically engineered bacteria comprise one or more gene sequence(s) which encode aspC from E. coli. In one embodiment, the genetically engineered bacteria comprise one or more gene sequence(s) which encode aspC and ipdC. In one embodiment, the genetically engineered bacteria comprise one or more gene sequence(s) which encode taal (L-tryptophan-pyruvate aminotransferase. In one embodiment, the genetically engineered bacteria comprise one or more gene sequence(s) which encode taal from Arabidopsis thaliana. In one embodiment, the genetically engineered bacteria comprise one or more gene sequence(s) which encode taal and ipdC. In one embodiment, the genetically engineered bacteria comprise one or more gene sequence(s) which encode staO (L-tryptophan oxidase). In one embodiment, the genetically engineered bacteria comprise one or more gene sequence(s) which encode staO from streptomyces sp. TP-A0274. In one embodiment, the genetically engineered bacteria comprise one or more gene sequence(s) which encode staO and ipdC. In one embodiment, the genetically engineered bacteria comprise one or more gene sequence(s) which encode trpDH (Tryptophan dehydrogenase). In one embodiment, the genetically engineered bacteria comprise one or more gene sequence(s) which encode trpDH from Nostoc punctiforme NIES-2108. In one embodiment, the genetically engineered bacteria comprise one or more gene sequence(s) which encode trpDH and ipdC. In one embodiment, the genetically engineered bacteria comprise one or more gene sequence(s) which encode one or more of aro9 or aspC or taal or staO or trpDH. In one embodiment, the genetically engineered bacteria comprise one or more gene sequence(s) which encode one or more of aro9 or aspC or taal or staO or trpDH and ipdC.

Further exemplary gene cassettes for the production of produce indole-3-acetaldehyde and FICZ from tryptophan are shown. In one embodiment, the genetically engineered bacteria comprise one or more gene sequence(s) which encode tdc (Tryptophan decarboxylase). In one embodiment, the genetically engineered bacteria comprise one or more gene sequence(s) which encode tdc from Catharanthus roseus. In one embodiment, the genetically engineered bacteria comprise one or more gene sequence(s) which encode tdc from Clostridium sporogenes. In one embodiment, the genetically engineered bacteria comprise one or more gene sequence(s) which encode tynA (Monoamine oxidase). In one embodiment, the genetically engineered bacteria comprise one or more gene sequence(s) which encode tynA from E. coli. In one embodiment, the genetically engineered bacteria comprise one or more gene sequence(s) which encode tdc (e.g., from Clostridium sporogenes) and tynA.

In any of these embodiments, the genetically engineered bacteria which produce produce indole-3-acetaldehyde and FICZ also optionally comprise one or more gene sequence(s) comprising one or more enzymes for tryptophan production, and gene deletions/or mutations as depicted and described described elsewhere herein. In some embodiments, the genetically engineered bacteria which produce indole-3-acetaldehyde and FICZ also optionally comprise one or more gene sequence(s) which encode one or more transporter(s) as described herein, through which tryptophan can be imported. Optionally, in some embodiments, the genetically engineered bacteria which produce indole-3-acetaldehyde and FICZ also optionally comprise one or more gene sequence(s) which encode an exporter as described herein, which can export tryptophan or any of its metabolites.

In some embodiments, the genetically engineered bacteria are capable of producing Indole-3-aldehyde under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with inflammation or an inflammatory response, or in the presence of some other metabolite that may or may not be present in the gut, such as arabinose.

(h) Indole Acetic Acid

In some embodiments, the genetically engineered bacteria comprise one or more gene cassettes which convert tryptophan to Indole-3-aldehyde and Indole Acetic Acid, e.g., via a tryptophan aminotransferase cassette. In some embodiments, the genetically engineered bacteria take up tryptophan through an endogenous or exogenous transporter, and further produce Indole-3-aldehyde and Indole Acetic Acid from tryptophan. In some embodiments, the genetically engineered bacteria optionally comprise a tryptophan and/or indole metabolite exporter.

The genetically engineered bacteria may comprise any suitable gene for producing Indole-3-aldehyde and/or Indole Acetic Acid and/or Tryptamine. In some embodiments, the gene for producing kynurenine is modified and/or mutated, e.g., to enhance stability, increase Indole-3-aldehyde and/or Indole Acetic Acid and/or Tryptamine production, and/or increase anti-inflammatory potency under inducing conditions. In some embodiments, the engineered bacteria also have enhanced uptake or import of tryptophan, e.g., comprise a transporter or other mechanism for increasing the uptake of tryptophan into the bacterial cell, as discussed in detail above. In some embodiments, the engineered bacteria also have enhanced export of a indole tryptophan metabolite, e.g., comprise an exporter or other mechanism for increasing the uptake of tryptophan into the bacterial cell, as discussed in detail above. In some embodiments, the genetically engineered bacteria are capable of producing Indole-3-aldehyde and/or Indole Acetic Acid and/or Tryptamine under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with inflammation or an inflammatory response, or in the presence of some other metabolite that may or may not be present in the gut, such as arabinose.

In one embodiment, the genetically engineered bacteria comprise one or more gene sequence(s) which encode one or more tryptophan catabolism enzymes, which produce indole-3-acetic acid.

Non-limiting example of such gene sequence(s) are shown.

In one embodiment, the genetically engineered bacteria comprise one or more gene sequence(s) which encode aro9 (L-tryptophan aminotransferase). In one embodiment, the genetically engineered bacteria comprise one or more gene sequence(s) which encode aro9 from S. cerevisae). In one embodiment, the genetically engineered bacteria comprise one or more gene sequence(s) which encode aspC (aspartate aminotransferase), In one embodiment, the genetically engineered bacteria comprise one or more gene sequence(s) which encode aspC from E. coli. In one embodiment, the genetically engineered bacteria comprise one or more gene sequence(s) which encode taal (L-tryptophan-pyruvate aminotransferase. In one embodiment, the genetically engineered bacteria comprise one or more gene sequence(s) which encode taal from Arabidopsis thaliana). In one embodiment, the genetically engineered bacteria comprise one or more gene sequence(s) which encode staO (L-tryptophan oxidase). In one embodiment, the genetically engineered bacteria comprise one or more gene sequence(s) which encode staO from streptomyces sp. TP-A0274). In one embodiment, the genetically engineered bacteria comprise one or more gene sequence(s) which encode trpDH (Tryptophan dehydrogenase). In one embodiment, the genetically engineered bacteria comprise one or more gene sequence(s) which encode trpDH from Nostoc punctiforme NIES-2108). In one embodiment, the genetically engineered bacteria comprise one or more gene sequence(s) which encode iad1 (Indole-3-acetaldehyde dehydrogenase). In one embodiment, the genetically engineered bacteria comprise one or more gene sequence(s) which encode iad1 from Ustilago maydis. In one embodiment, the genetically engineered bacteria comprise one or more gene sequence(s) which encode AAO1 (Indole-3-acetaldehyde oxidase). In one embodiment, the genetically engineered bacteria comprise one or more gene sequence(s) which encode AAO1 from Arabidopsis thaliana. In one embodiment, the genetically engineered bacteria comprise one or more gene sequence(s) which encode ipdC (Indole-3-pyruvate decarboxylase, e.g., from Enterobacter cloacae). In one embodiment, the genetically engineered bacteria comprise one or more gene sequence(s) which encode ipdC (Indole-3-pyruvate decarboxylase, e.g., from Enterobacter cloacae) in combination with one or more sequences encoding enzymes selected from aro9 and/or aspC and/or taal and/or staO and/or trpDH. In one embodiment, the genetically engineered bacteria comprise one or more gene sequence(s) which encode ipdC (Indole-3-pyruvate decarboxylase, e.g., from Enterobacter cloacae) in combination with one or more sequences encoding enzymes selected from iad1 and/or aao1. In one embodiment, the genetically engineered bacteria comprise one or more gene sequence(s) which encode ipdC (Indole-3-pyruvate decarboxylase, e.g., from Enterobacter cloacae) in combination with one or more sequences encoding enzymes selected from aro9 and/or aspC and/or taal and/or staO and in combination with one or more sequences encoding enzymes selected from iad1 and/or aao1.

In one embodiment, the genetically engineered bacteria comprise one or more gene sequence(s) which encode tdc (Tryptophan decarboxylase). In one embodiment, the genetically engineered bacteria comprise one or more gene sequence(s) which encode tdc from Catharanthus roseus. In one embodiment, the genetically engineered bacteria comprise one or more gene sequence(s) which encode tdc from Clostridium sporogenes. In one embodiment, the genetically engineered bacteria comprise one or more gene sequence(s) which encode tynA (Monoamine oxidase). In one embodiment, the genetically engineered bacteria comprise one or more gene sequence(s) which encode tynA from E. coli). In one embodiment, the genetically engineered bacteria comprise one or more gene sequence(s) which encode iad1 (Indole-3-acetaldehyde dehydrogenase). In one embodiment, the genetically engineered bacteria comprise one or more gene sequence(s) which encode iad1 from Ustilago maydis). In one embodiment, the genetically engineered bacteria comprise one or more gene sequence(s) which encode AAO1 (Indole-3-acetaldehyde oxidase). In one embodiment, the genetically engineered bacteria comprise one or more gene sequence(s) which encode AAO1 from Arabidopsis thaliana). In one embodiment, the genetically engineered bacteria comprise one or more gene sequence(s) which encode tdc and tynA. In one embodiment, the genetically engineered bacteria comprise one or more gene sequence(s) which encode tdc and one or more sequence(s) selected from iad1 and/or aao1. In one embodiment, the genetically engineered bacteria comprise one or more gene sequence(s) which encode tynA and one or more sequence(s) selected from iad1 and/or aao1. In one embodiment, the genetically engineered bacteria comprise one or more gene sequence(s) which encode tdc and tynA and one or more sequence(s) selected from iad1 and/or aao1.

In one embodiment, the genetically engineered bacteria comprise one or more gene sequence(s) which encode trpDH (Tryptophan dehydrogenase). In one embodiment, the genetically engineered bacteria comprise one or more gene sequence(s) which encode trpDH from Nostoc punctiforme NIES-2108. In one embodiment, the genetically engineered bacteria comprise one or more gene sequence(s) which encode ipdC (Indole-3-pyruvate decarboxylase, e.g., from Enterobacter cloacae). In one embodiment, the genetically engineered bacteria comprise one or more gene sequence(s) which encode iad1 (Indole-3-acetaldehyde dehydrogenase). In one embodiment, the genetically engineered bacteria comprise one or more gene sequence(s) which encode iad1 from Ustilago maydis. In one embodiment, the genetically engineered bacteria comprise one or more gene sequence(s) which encode one or more of trpDH and/or ipdC and/or iad1. In one embodiment, the genetically engineered bacteria comprise one or more gene sequence(s) which encode one or more of trpDH and ipdC and iad1.

In one embodiment, the genetically engineered bacteria comprise one or more gene sequence(s) which encode yuc2 (indole-3-pyruvate monooxygenase). In one embodiment, the genetically engineered bacteria comprise one or more gene sequence(s) which encode yuc2 from Enterobacter cloacae. In one embodiment, the genetically engineered bacteria comprise one or more gene sequence(s) which encode aro9 (L-tryptophan aminotransferase). In one embodiment, the (L-tryptophan aminotransferase is from S. cerevisiae. In one embodiment, the genetically engineered bacteria comprise one or more gene sequence(s) which encode aro9 and yuc2. In one embodiment, the genetically engineered bacteria comprise one or more gene sequence(s) which encode aspC (aspartate aminotransferase. In one embodiment, the genetically engineered bacteria comprise one or more gene sequence(s) which encode aspC from E. coli. In one embodiment, the genetically engineered bacteria comprise one or more gene sequence(s) which encode aspC and yuc2. In one embodiment, the genetically engineered bacteria comprise one or more gene sequence(s) which encode taal (L-tryptophan-pyruvate aminotransferase. In one embodiment, the genetically engineered bacteria comprise one or more gene sequence(s) which encode taal from Arabidopsis thaliana. In one embodiment, the genetically engineered bacteria comprise one or more gene sequence(s) which encode taal and yuc2. In one embodiment, the genetically engineered bacteria comprise one or more gene sequence(s) which encode staO (L-tryptophan oxidase). In one embodiment, the genetically engineered bacteria comprise one or more gene sequence(s) which encode staO from streptomyces sp. TP-A0274. In one embodiment, the genetically engineered bacteria comprise one or more gene sequence(s) which encode staO and yuc2. In one embodiment, the genetically engineered bacteria comprise one or more gene sequence(s) which encode trpDH (Tryptophan dehydrogenase). In one embodiment, the genetically engineered bacteria comprise one or more gene sequence(s) which encode trpDH from Nostoc punctiforme NIES-2108. In one embodiment, the genetically engineered bacteria comprise one or more gene sequence(s) which encode trpDH and yuc2. In one embodiment, the genetically engineered bacteria comprise one or more gene sequence(s) which encode one or more of aro9 or aspC or taal or staO or trpDH. In one embodiment, the genetically engineered bacteria comprise one or more gene sequence(s) which encode one or more of aro9 or aspC or taal or staO or trpDH and yuc2.

In one embodiment, the genetically engineered bacteria comprise one or more gene sequence(s) which encode IaaM (Tryptophan 2-monooxygenase). In one embodiment, the genetically engineered bacteria comprise one or more gene sequence(s) which encode IaaM from Pseudomonas savastanoi). In one embodiment, the genetically engineered bacteria comprise one or more gene sequence(s) which encode iaaH (Indoleacetamide hydrolase). In one embodiment, the genetically engineered bacteria comprise one or more gene sequence(s) which encode iaaH from Pseudomonas savastanoi). In one embodiment, the genetically engineered bacteria comprise one or more gene sequence(s) which encode IaaM and iaaH.

In one embodiment, the genetically engineered bacteria comprise one or more gene sequence(s) which encode cyp71a13 (indoleacetaldoxime dehydratase). In one embodiment, the genetically engineered bacteria comprise one or more gene sequence(s) which encode cyp71a13 from Arabidopis thaliana. In one embodiment, the genetically engineered bacteria comprise one or more gene sequence(s) which encode nitl (Nitrilase). In one embodiment, the genetically engineered bacteria comprise one or more gene sequence(s) which encode nit1 from Arabidopsis thaliana. In one embodiment, the genetically engineered bacteria comprise one or more gene sequence(s) which encode iaaH (Indoleacetamide hydrolase). In one embodiment, the genetically engineered bacteria comprise one or more gene sequence(s) which encode iaaH from Pseudomonas savastanoi),In one embodiment, the genetically engineered bacteria comprise one or more gene sequence(s) which encode cyp79B2 (tryptophan N-monooxygenase). In one embodiment, the genetically engineered bacteria comprise one or more gene sequence(s) which encode cyp79B2 from Arabidopsis thaliana. In one embodiment, the genetically engineered bacteria comprise one or more gene sequence(s) which encode cyp79B2 and cyp71a13. In one embodiment, the genetically engineered bacteria comprise one or more gene sequence(s) which encode cyp79B2 from Arabidopsis thaliana. In one embodiment, the genetically engineered bacteria comprise one or more gene sequence(s) which encode cyp79B2 and nitl and/or iaaH. In one embodiment, the genetically engineered bacteria comprise one or more gene sequence(s) which encode cyp79B3 (tryptophan N-monooxygenase). In one embodiment, the genetically engineered bacteria comprise one or more gene sequence(s) which encode cyp79B3 from Arabidopsis thaliana. In one embodiment, the genetically engineered bacteria comprise one or more gene sequence(s) which encode cyp79B3 and cyp71a13. In one embodiment, the genetically engineered bacteria comprise one or more gene sequence(s) which encode cyp79B3 and cyp71a13 and nitl and/or iaaH. In one embodiment, the genetically engineered bacteria comprise one or more gene sequence(s) which encode cyp79B3, cyp79B2 and cyp71a13. In one embodiment, the genetically engineered bacteria comprise one or more gene sequence(s) which encode cyp79B3, cyp79B2 and cyp71a13, and nitl and/or iaaH. In one embodiment, the genetically engineered bacteria comprise one or more gene sequence(s) which encode cyp79B3 from Arabidopsis thaliana. In one embodiment, the genetically engineered bacteria comprise one or more gene sequence(s) which encode cyp79B3 and cyp71a13 and nitl and iaaH. In one embodiment, the genetically engineered bacteria comprise one or more gene sequence(s) which encode cyp79B3, cyp79B2 and cyp71a13 and nitl and iaaH.

In any of these embodiments, the genetically engineered bacteria which produce indole acetic acid also optionally comprise one or more gene sequence(s) comprising one or more enzymes for tryptophan production, and gene deletions/or mutations as depicted and described elsewhere herein. In some embodiments, the genetically engineered bacteria which produce indole acetic acid also optionally comprise one or more gene sequence(s) which encode one or more transporter(s) as described herein, through which tryptophan can be imported. Optionally, in some embodiments, the genetically engineered bacteria which produce indole acetic acid also optionally comprise one or more gene sequence(s) which encode an exporter as described herein, which can export tryptophan or any of its metabolites.

In some embodiments, the genetically engineered bacteria are capable of producing Indole Acetic Acid and under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

(I) Indole-3-acetonitrile

In one embodiment, the genetically engineered bacteria comprise one or more gene sequence(s) which encode one or more tryptophan catabolism enzymes, which produce indole-3-acetonitrile from tryptophan.

In one embodiment, the genetically engineered bacteria comprise one or more gene sequence(s) which encode cyp79B2 (tryptophan N-monooxygenase). In one embodiment, the genetically engineered bacteria comprise one or more gene sequence(s) which encode cyp79B2 from Arabidopsis thaliana. In one embodiment, the genetically engineered bacteria comprise one or more gene sequence(s) which encode cyp71a13 (indoleacetaldoxime dehydratase). In one embodiment, the genetically engineered bacteria comprise one or more gene sequence(s) which encode cyp71a13 from Arabidopis thaliana. In one embodiment, the genetically engineered bacteria comprise one or more gene sequence(s) which encode cyp79B2 and cyp71a13.

In one embodiment, the genetically engineered bacteria comprise one or more gene sequence(s) which encode cyp79B3 (tryptophan N-monooxygenase) In one embodiment, the genetically engineered bacteria comprise one or more gene sequence(s) which encode cyp79B3 from Arabidopsis thaliana. In one embodiment, the genetically engineered bacteria comprise one or more gene sequence(s) which encode cyp79B3 and cyp71a13. In one embodiment, the genetically engineered bacteria comprise one or more gene sequence(s) which encode cyp79B3, cyp79B2 and cyp71a13.

In any of these embodiments, the genetically engineered bacteria which produce indole-3-acetonitrile from tryptophan also optionally comprise one or more gene sequence(s) comprising one or more enzymes for tryptophan production, and gene deletions/or mutations as depicted and described elsewhere herein. In some embodiments, the genetically engineered bacteria which produce indole-3-acetonitrile from tryptophan also optionally comprise one or more gene sequence(s) which encode one or more transporter(s) as described herein, through which tryptophan can be imported. Optionally, in some embodiments, the genetically engineered bacteria which produce indole-3-acetonitrile from tryptophan also optionally comprise one or more gene sequence(s) which encode an exporter as described herein, which can export tryptophan or any of its metabolites.

(j) Indole-3-Propionic Acid (IPA)

In one embodiment, the genetically engineered bacteria comprise one or more gene sequence(s) which encode one or more tryptophan catabolism enzymes, which produce indole-3-propionic acid from tryptophan.

In some embodiments, the genetically engineered bacteria comprise one or more gene sequences encoding tryptophan ammonia lyase. In some embodiments, the genetically engineered bacteria comprise one or more gene sequences encoding tryptophan ammonia lyase from Rubrivivax benzoatilyticus. In some embodiments, the genetically engineered bacteria comprise one or more gene sequences encoding indole-3-acrylate reductase. In some embodiments, the genetically engineered bacteria comprise one or more gene sequences encoding indole-3-acrylate reductase from Clostridum botulinum. In some embodiments, the genetically engineered bacteria comprise one or more gene sequences encoding a tryptophan ammonia lyase and an indole-3-acrylate reductase. In some embodiments, the indole-3-propionate-producing strain optionally produces tryptophan from a chorismate precursor, and the strain optionally comprises additional circuits for tryptophan production and/or tryptophan uptake/transport s described herein.

The genetically engineered bacteria comprise a circuit, comprising trpDH (Tryptophan dehydrogenase, e.g., from Nostoc punctiforme NIES-2108, which produces (indol-3yl)pyruvate from tryptophan), fldA (indole-3-propionyl-CoA:indole-3-lactate CoA transferase, e.g., from Clostridium sporogenes, which converts converts indole-3-lactate and indol-3-propionyl-CoA to indole-3-propionic acid and indole-3-lactate-CoA), fldB and fldC (indole-3-lactate dehydratase e.g., from Clostridium sporogenes, which converts indole-3-lactate-CoA to indole-3-acrylyl-CoA) fldD and/or AcuI: (indole-3-acrylyl-CoA reductase, e.g., from Clostridium sporogenes and/or acrylyl-CoA reductase, e.g., from Rhodobacter sphaeroides, which convert indole-3-acrylyl-CoA to indole-3-propionyl-CoA). The circuits further comprise fldH1 and/or fldH2 (indole-3-lactate dehydrogenase 1 and/or 2, e.g., from Clostridium sporogenes), which converts (indol-3-yl)pyruvate into indole-3-lactate).

In some embodiments, the genetically engineered bacteria comprise one or more gene sequences encoding trpDH (Tryptophan dehydrogenase). In some embodiments, the genetically engineered bacteria comprise one or more gene sequences encoding trpDH from Nostoc punctiforme NIES-2108. In some embodiments, the genetically engineered bacteria comprise one or more gene sequences encoding fldA (indole-3-propionyl-CoA:indole-3-lactate CoA transferase). In some embodiments, the genetically engineered bacteria comprise one or more gene sequences encoding fldA from Clostridium sporogenes. In some embodiments, the genetically engineered bacteria comprise one or more gene sequences encoding fldB and fldC (indole-3-lactate dehydratase). In some embodiments, the genetically engineered bacteria comprise one or more gene sequences encoding fldB and fldC Clostridium sporogenes. In some embodiments, the genetically engineered bacteria comprise one or more gene sequences encoding fldD (indole-3-acrylyl-CoA reductase). In some embodiments, the genetically engineered bacteria comprise one or more gene sequences encoding fldD from Clostridium sporogenes. In some embodiments, the genetically engineered bacteria comprise one or more gene sequences encoding AcuI (acrylyl-CoA reductase). In some embodiments, the genetically engineered bacteria comprise one or more gene sequences encoding AcuI from Rhodobacter sphaeroides. In some embodiments, the genetically engineered bacteria comprise one or more gene sequences encoding fldH1 (3-lactate dehydrogenase 1). In some embodiments, the genetically engineered bacteria comprise one or more gene sequences encoding fldH1 from Clostridium sporogenes. In some embodiments, the genetically engineered bacteria comprise one or more gene sequences encoding fldH2 (indole-3-lactate dehydrogenase 2). In some embodiments, the genetically engineered bacteria comprise one or more gene sequences encoding fldH2 from Clostridium sporogenes). In some embodiments, the genetically engineered bacteria comprise one or more gene sequences encoding trpDH and/or fldA and/or fldB and/or flD and/or fldH1. In some embodiments, the genetically engineered bacteria comprise one or more gene sequences encoding trpDH and/or fldA and/or fldB and/or flD and/or fldH2. In some embodiments, the genetically engineered bacteria comprise one or more gene sequences encoding trpDH and/or fldA and/or fldB and/or acul and/or fldH1. In some embodiments, the genetically engineered bacteria comprise one or more gene sequences encoding trpDH and/or fldA and/or fldB and/or acul and/or fldH2. In some embodiments, the genetically engineered bacteria comprise one or more gene sequences encoding trpDH and fldA and fldB and flD and fldH1. In some embodiments, the genetically engineered bacteria comprise one or more gene sequences encoding trpDH and fldA and fldB and flD and fldH2. In some embodiments, the genetically engineered bacteria comprise one or more gene sequences encoding trpDH and fldA and fldB and acul and fldH1. In some embodiments, the genetically engineered bacteria comprise one or more gene sequences encoding trpDH and fldA and fldB and acul and fldH2.

In any of these embodiments, the genetically engineered bacteria which produce indole-3-propionic acid also optionally comprise one or more gene sequence(s) comprising one or more enzymes for tryptophan production, and gene deletions/or mutations as depicted and described elsewhere herein. In some embodiments, the genetically engineered bacteria which produce indole-3-propionic acid also optionally comprise one or more gene sequence(s) which encode one or more transporter(s) as described herein, through which tryptophan can be imported. Optionally, in some embodiments, the genetically engineered bacteria which produce indole-3-propionic acid also optionally comprise one or more gene sequence(s) which encode an exporter as described herein, which can export tryptophan or any of its metabolites.

In certain embodiments, the genetically engineered bacteria comprise one or more gene sequence(s) encoding one or more enzymes for the production of tryptophan metabolites. In some embodiments, the genetically engineered bacteria comprise one or more gene sequence(s) encoding 1, 2, 3, 4, 5, 6, 7, 8, 9, 10 different tryptophan metabolites. In certain embodiments the bacteria comprise one or more gene sequence(s) encoding one or more enzymes for the production of tryptophan metabolites selected from tryptamine and/or indole-3 acetaladehyde, indole-3acetonitrile, kynurenine, kynurenic acid, indole, indole acetic acid FICZ, indole-3-propionic acid.

In some embodiments, the genetically engineered bacteria are capable of producing Indole-3-aldehyde and/or Indole Acetic Acid and/or Tryptamine under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

(k) Indole

In one embodiment, the genetically engineered bacteria comprise one or more gene sequence(s) which encode one or more tryptophan catabolism enzymes, which produce indole from tryptophan. Non-limiting example of such gene sequence(s) are shown and described elsewhere herein. In one embodiment, the genetically engineered bacteria comprise one or more gene sequence(s) which encode tnaA (tryptophanase). In one embodiment, the genetically engineered bacteria comprise one or more gene sequence(s) which encode tnaA from E. coli.

In any of these embodiments, the genetically engineered bacteria which produce indole from tryptophan also optionally comprise one or more gene sequence(s) comprising one or more enzymes for tryptophan production, and gene deletions/or mutations as depicted and described elsewhere herein. In some embodiments, the genetically engineered bacteria which produce indole from tryptophan also optionally comprise one or more gene sequence(s) which encode one or more transporter(s) as described herein, through which tryptophan can be imported. Optionally, in some embodiments, the genetically engineered bacteria which produce indole from tryptophan also optionally comprise one or more gene sequence(s) which encode an exporter as described herein, which can export tryptophan or any of its metabolites.

In some embodiments, the genetically engineered bacteria are capable of producing Indole-3-acetonitrile under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

(l) Other Indole Metabolites

In one embodiment, the genetically engineered bacteria comprise one or more gene sequence(s) which encode one or more tryptophan catabolism enzymes, which produce indole-3-carbinol, indole-3-aldehyde, 3,3′ diindolylmethane (DIM), indolo(3,2-b) carbazole (ICZ) from indole glucosinolate taken up through the diet. Non-limiting example of such gene sequence(s) are shown FIG. 16G and described elsewhere herein. In one embodiment, the genetically engineered bacteria comprise one or more gene sequence(s) which encode pne2 (myrosinase). In one embodiment, the genetically engineered bacteria comprise one or more gene sequence(s) which encode pne2 from Arabidopsis thaliana.

In any of these embodiments, the genetically engineered bacteria also optionally comprise one or more gene sequence(s) comprising one or more enzymes for tryptophan production, and gene deletions/or mutations as depicted and described elsewhere herein. In some embodiments, the genetically engineered bacteria also optionally comprise one or more gene sequence(s) which encode one or more transporter(s) as described herein, through which tryptophan can be imported. Optionally, in some embodiments, the genetically engineered bacteria also optionally comprise one or more gene sequence(s) which encode an exporter as described herein, which can export tryptophan or any of its metabolites.

In some embodiments, the genetically engineered bacteria are capable of producing these metabolites under inducing conditions, e.g., under a condition(s) associated with inflammation. In some embodiments, the genetically engineered bacteria are capable of producing kynurenine in low-oxygen conditions, in the presence of certain molecules or metabolites, in the presence of molecules or metabolites associated with liver damage, metabolic disease, inflammation or an inflammatory response, or in the presence of some other metabolite that may or may not be present in the gut, such as arabinose.

B. Tryptophan Catabolic Pathway Enzymes

Table 15 comprises polypeptide sequences of such enzymes which are encoded by the genetically engineered bacteria of the disclosure.

TABLE 15
Tryptophan Pathway Catabolic Enzymes

| Description | Sequence |
|---|---|
| TDC: Tryptophan | MGSIDSTNVAMSNSPVGEFKPLEAEEFRKQAHRMVDFIADYY |
| decarboxylase from | KNVETYPVLSEVEPGYLRKRIPETAPYLPEPLDDIMKDIQKDII |
| Catharanthus roseus | PGMTNWMSPNFYAFFPATVSSAAFLGEMLSTALNSVGFTWV |
| SEQ ID NO: 99 | SSPAATELEMIVMDWLAQILKLPKSFMFSGTGGGVIQNTTSES |
| | ILCTIIAARERALEKLGPDSIGKLVCYGSDQTHTMFPKTCKLA |
| | GIYPNNIRLIPTTVETDFGISPQVLRKMVEDDVAAGYVPLFLC |
| | ATLGTTSTTATDPVDSLSEIANEFGIWIHVDAAYAGSACICPEF |
| | RHYLDGIERVDSLSLSPHKWLLAYLDCTCLWVKQPHLLLRAL |
| | TTNPEYLKNKQSDLDKVVDFKNWQIATGRKFRSLKLWLILRS |
| | YGVVNLQSHIRSDVAMGKMFEEWVRSDSRFEIVVPRNFSLVC |
| | FRLKPDVSSLHVEEVNKKLLDMLNSTGRVYMTHTIVGGIYML |
| | RLAVGSSLTEEHHVRRVWDLIQKLTDDLLKEA |
| TDC: Tryptophan | MKFWRKYTQQEMDEKITESLEKTLNYDNTKTIGIPGTKLDDT |
| decarboxylase from | VFYDDHSFVKHSPYLRTFIQNPNHIGCHTYDKADILFGGTFDIE |
| Clostridium | RELIQLLAIDVLNGNDEEFDGYVTQGGTEANIQAMWVYRNY |
| sporogenes | FKKERKAKHEEIAIITSADTHYSAYKGSDLLNIDIIKVPVDFYS |
| SEQ ID NO: XXXX | RKIQENTLDSIVKEAKEIGKKYFIVISNMGTTMFGSVDDPDLY |
| | ANIFDKYNLEYKIHVDGAFGGFIYPIDNKECKTDFSNKNVSSIT |
| | LDGHKMLQAPYGTGIFVSRKNLIHNTLTKEATYIENLDVTLSG |
| | SRSGSNAVAIWMVLASYGPYGWMEKINKLRNRTKWLCKQL |
| | NDMRIKYYKEDSMNIVTIEEQYVNKEIAEKYFLVPEVHNPTN |
| | NWYKIVVMEHVELDILNSLVYDLRKFNKEHLKAM |
| TYNA: Monoamine | MGSPSLYSARKTTLALAVALSFAWQAPVFAHGGEAHMVPM |
| oxidase from E. coli | DKTLKEFGADVQWDDYAQLFTLIKDGAYVKVKPGAQTAIVN |
| SEQ ID NO: 100 | GQPLALQVPVVMKDNKAWVSDTFINDVFQSGLDQTFQVEKR |
| | PHPLNALTADEIKQAVEIVKASADFKPNTRFTEISLLPPDKEAV |
| | WAFALENKPVDQPRKADVIMLDGKHIIEAVVDLQNNKLLSW |
| | QPIKDAHGMVLLDDFASVQNIINNSEEFAAAVKKRGITDAKK |
| | VITTPLTVGYFDGKDGLKQDARLLKVISYLDVGDGNYWAHPI |
| | ENLVAVVDLEQKKIVKIEEGPVVPVPMTARPFDGRDRVAPAV |
| | KPMQIIEPEGKNYTITGDMIHWRNWDFHLSMNSRVGPMISTV |
| | TYNDNGTKRKVMYEGSLGGMIVPYGDPDIGWYFKAYLDSGD |
| | YGMGTLTSPIARGKDAPSNAVLLNETIADYTGVPMEIPRAIAV |
| | FERYAGPEYKHQEMGQPNVSTERRELVVRWISTVGNYDYIFD |
| | WIFHENGTIGIDAGATGIEAVKGVKAKTMHDETAKDDTRYGT |
| | LIDHNIVGTTHQHIYNFRLDLDVDGENNSLVAMDPVVKPNTA |
| | GGPRTSTMQVNQYNIGNEQDAAQKFDPGTIRLLSNPNKENRM |
| | GNPVSYQIIPYAGGTHPVAKGAQFAPDEWIYHRLSFMDKQLW |
| | VTRYHPGERFPEGKYPNRSTHDTGLGQYSKDNESLDNTDAV |
| | VWMTTGTTHVARAEEWPIMPTEWVHTLLKPWNFFDETPTLG |
| | ALKKDK |
| AAO1: Indole-3- | MGEKAIDEDKVEAMKSSKTSLVFAINGQRFELELSSIDPSTTL |
| acetaldehyde oxidase | VDFLRNKTPFKSVKLGCGEGGCGACVVLLSKYDPLLEKVDEF |
| from Arabidopsis | TISSCLTLLCSIDGCSITTSDGLGNSRVGFHAVHERIAGFHATQ |
| thaliana | CGFCTPGMSVSMFSALLNADKSHPPPRSGFSNLTAVEAEKAV |
| SEQ ID NO: 101 | SGNLCRCTGYRPLVDACKSFAADVDIEDLGFNAFCKKGENRD |
| | EVLRRLPCYDHTSSHVCTFPEFLKKEIKNDMSLHSRKYRWSSP |
| | VSVSELQGLLEVENGLSVKLVAGNTSTGYYKEEKERKYERFI |
| | DIRKIPEFTMVRSDEKGVELGACVTISKAIEVLREEKNVSVLA |
| | KIATHMEKIANRFVRNTGTIGGNIMMAQRKQFPSDLATILVA |
| | AQATVKIMTSSSSQEQFTLEEFLQQPPLDAKSLLLSLEIPSWHS |
| | AKKNGSSEDSILLFETYRAAPRPLGNALAFLNAAFSAEVTEAL |
| | DGIVVNDCQLVFGAYGTKHAHRAKKVEEFLTGKVISDEVLM |
| | EAISLLKDEIVPDKGTSNPGYRSSLAVTFLFEFFGSLTKKNAKT |
| | TNGWLNGGCKEIGFDQNVESLKPEAMLSSAQQIVENQEHSPV |
| | GKGITKAGACLQASGEAVYVDDIPAPENCLYGAFIYSTMPLA |
| | RIKGIRFKQNRVPEGVLGIITYKDIPKGGQNIGTNGFFTSDLLF |
| | AEEVTHCAGQIIAFLVADSQKHADIAANLVVIDYDTKDLKPPI |
| | LSLEEAVENFSLFEVPPPLRGYPVGDITKGMDEAEHKILGSKIS |
| | FGSQYFFYMETQTALAVPDEDNCMVVYSSTQTPEFVHQTIAG |
| | CLGVPENNVRVITRRVGGGFGGKAVKSMPVAAACALAASK |
| | MQRPVRTYVNRKTDMITTGGRHPMKVTYSVGFKSNGKITAL |
| | DVEVLLDAGLTEDISPLMPKGIQGALMKYDWGALSFNVKVC |
| | KTNTVSRTALRAPGDVQGSYIGEAIIEKVASYLSVDVDEIRKV |
| | NLHTYESLRLFHSAKAGEFSEYTLPLLWDRIDEFSGFNKRRKV |
| | VEEFNASNKWRKRGISRVPAVYAVNMRSTPGRVSVLGDGSIV |
| | VEVQGIEIGQGLWTKVKQMAAYSLGLIQCGTTSDELLKKIRVI |
| | QSDTLSMVQGSMTAGSTTSEASSEAVRICCDGLVERLLPVKT |
| | ALVEQTGGPVTWDSLISQAYQQSINMSVSSKYMPDSTGEYLN |
| | YGIAASEVEVNVLTGETTILRTDIIYDCGKSLNPAVDLGQIEGA |
| | FVQGLGFFMLEEFLMNSDGLVVTDSTWTYKIPTVDTIPRQFN |
| | VEILNSGQHKNRVLSSKASGEPPLLLAASVHCAVRAAVKEAR |
| | KQILSWNSNKQGTDMYFELPVPATMPIVKEFCGLDVVEKYLE |
| | WKIQQRKNV |
| ARO9: L-tryptophan | MTAGSAPPVDYTSLKKNFQPFLSRRVENRSLKSFWDASDISD |
| aminotransferase | DVIELAGGMPNERFFPIESMDLKISKVPFNDNPKWHNSFTTAH |
| from S. cerevisae | LDLGSPSELPIARSFQYAETKGLPPLLHFVKDFVSRINRPAFSD |
| SEQ ID NO: 102 | ETESNWDVILSGGSNDSMFKVFETICDESTTVMIEEFTFTPAM |
| | SNVEATGAKVIPIKMNLTFDRESQGIDVEYLTQLLDNWSTGP |
| | YKDLNKPRVLYTIATGQNPTGMSVPQWKREKIYQLAQRHDF |
| | LIVEDDPYGYLYFPSYNPQEPLENPYHSSDLTTERYLNDFLMK |
| | SFLTLDTDARVIRLETFSKIFAPGLRLSFIVANKFLLQKILDLAD |
| | ITTRAPSGTSQAIVYSTIKAMAESNLSSSLSMKEAMFEGWIRW |
| | IMQIASKYNHRKNLTLKALYETESYQAGQFTVMEPSAGMFIII |
| | KINWGNFDRPDDLPQQMDILDKFLLKNGVKVVLGYKMAVCP |
| | NYSKQNSDFLRLTIAYARDDDQLIEASKRIGSGIKEFFDNYKS |
| aspC: aspartate | MFENITAAPADPILGLADLFRADERPGKINLGIGVYKDETGKT |
| aminotransferase | PVLTSVKKAEQYLLENETTKNYLGIDGIPEFGRCTQELLFGKG |
| from E. coli | SALINDKRARTAQTPGGTGALRVAADFLAKNTSVKRVWVSN |
| SEQ ID NO: 103 | PSWPNHKSVFNSAGLEVREYAYYDAENHTLDFDALINSLNEA |
| | QAGDVVLFHGCCHNPTGIDPTLEQWQTLAQLSVEKGWLPLF |
| | DFAYQGFARGLEEDAEGLRAFAAMHKELIVASSYSKNFGLYN |
| | ERVGACTLVAADSETVDRAFSQMKAAIRANYSNPPAHGASV |
| | VATILSNDALRAIWEQELTDMRQRIQRMRQLFVNTLQEKGAN |
| | RDFSFIIKQNGMFSFSGLTKEQVLRLREEFGVYAVASGRVNVA |
| | GMTPDNMAPLCEAIVAVL |
| TAA1: L-tryptophan- | MVKLENSRKPEKISNKNIPMSDFVVNLDHGDPTAYEEYWRK |
| pyruvate | MGDRCTVTIRGCDLMSYFSDMTNLCWFLEPELEDAIKDLHGV |
| aminotransferase | VGNAATEDRYIVVGTGSTQLCQAAVHALSSLARSQPVSVVA |
| from Arabidopsis | AAPFYSTYVEETTYVRSGMYKWEGDAWGFDKKGPYIELVTS |
| thaliana | PNNPDGTIRETVVNRPDDDEAKVIHDFAYYWPHYTPITRRQD |
| SEQ ID NO: 104 | HDIMLFTFSKITGHAGSRIGWALVKDKEVAKKMVEYIIVNSIG |
| | VSKESQVRTAKILNVLKETCKSESESENFFKYGREMMKNRWE |
| | KLREVVKESDAFTLPKYPEAFCNYFGKSLESYPAFAWLGTKE |
| | ETDLVSELRRHKVMSRAGERCGSDKKHVRVSMLSREDVFNV |
| | FLERLANMKLIKSIDL |
| STAO: L-tryptophan | MTAPLQDSDGPDDAIGGPKQVTVIGAGIAGLVTAYELERLGH |
| oxidase from | HVQIIEGSDDIGGRIHTHRFSGAGGPGPFAEMGAMRIPAGHRL |
| streptomyces sp. TP- | TMHYIAELGLQNQVREFRTLFSDDAAYLPSSAGYLRVREAHD |
| A0274 | TLVDEFATGLPSAHYRQDTLLFGAWLDASIRAIAPRQFYDGL |
| SEQ ID NO: 105 | HNDIGVELLNLVDDIDLTPYRCGTARNRIDLHALFADHPRVR |
| | ASCPPRLERFLDDVLDETSSSIVRLKDGMDELPRRLASRIRGKI |
| | SLGQEVTGIDVHDDTVTLTVRQGLRTVTRTCDYVVCTIPFTVL |
| | RTLRLTGFDQDKLDIVHETKYWPATKIAFHCREPFWEKDGIS |
| | GGASFTGGHVRQTYYPPAEGDPALGAVLLASYTIGPDAEALA |
| | RMDEAERDALVAKELSVMHPELRRPGMVLAVAGRDWGARR |
| | WSRGAATVRWGQEAALREAERRECARPQKGLFFAGEHCSSK |
| | PAWIEGAIESAIDAAHEIEWYEPRASRVFAASRLSRSDRSA |
| ipdC: Indole-3- | MRTPYCVADYLLDRLTDCGADHLFGVPGDYNLQFLDHVIDS |
| pyruvate | PDICWVGCANELNASYAADGYARCKGFAALLTTFGVGELSA |
| decarboxylase from | MNGIAGSYAEHVPVLHIVGAPGTAAQQRGELLHHTLGDGEFR |
| Enterobacter cloacae | HFYHMSEPITVAQAVLTEQNACYEIDRVLTTMLRERRPGYLM |
| SEQ ID NO: 106 | LPADVAKKAATPPVNALTHKQAHADSACLKAFRDAAENKLA |
| | MSKRTALLADFLVLRHGLKHALQKWVKEVPMAHATMLMG |
| | KGIFDERQAGFYGTYSGSASTGAVKEAIEGADTVLCVGTRFT |
| | DTLTAGFTHQLTPAQTIEVQPHAARVGDVWFTGIPMNQAIET |
| | LVELCKQHVHAGLMSSSSGAIPFPQPDGSLTQENFWRTLQTFI |
| | RPGDIILADQGTSAFGAIDLRLPADVNFIVQPLWGSIGYTLAA |
| | AFGAQTACPNRRVIVLTGDGAAQLTIQELGSMLRDKQHPIILV |
| | LNNEGYTVERAIHGAEQRYNDIALWNWTHIPQALSLDPQSEC |
| | WRVSEAEQLADVLEKVAHHERLSLIEVMLPKADIPPLLGALT |
| | KALEACNNA |
| IAD1: Indole-3- | MPTLNLDLPNGIKSTIQADLFINNKFVPALDGKTFATINPSTGK |
| acetaldehyde | EIGQVAEASAKDVDLAVKAAREAFETTWGENTPGDARGRLLI |
| dehydrogenase from | KLAELVEANIDELAAIESLDNGKAFSIAKSFDVAAVAANLRY |
| Ustilago maydis | YGGWADKNHGKVMEVDTKRLNYTRHEPIGVCGQIIPWNFPL |
| SEQ ID NO: 107 | LMFAWKLGPALATGNTIVLKTAEQTPLSAIKMCELIVEAGFPP |
| | GVVNVISGFGPVAGAAISQHMDIDKIAFTGSTLVGRNIMKAA |
| | ASTNLKKVTLELGGKSPNIIFKDADLDQAVRWSAFGIMFNHG |
| | QCCCAGSRVYVEESIYDAFMEKMTAHCKALQVGDPFSANTF |
| | QGPQVSQLQYDRIMEYIESGKKDANLALGGVRKGNEGYFIEP |
| | TIFTDVPHDAKIAKEEIFGPVVVVSKFKDEKDLIRIANDSIYGL |
| | AAAVFSRDISRAIETAHKLKAGTVWVNCYNQLIPQVPFGGYK |
| | ASGIGRELGEYALSNYTNIKAVHVNLSQPAPI |
| YUC2: indole-3- | MEFVTETLGKRIHDPYVEETRCLMIPGPIIVGSGPSGLATAACL |
| pyruvate | KSRDIPSLILERSTCIASLWQHKTYDRLRLHLPKDFCELPLMPF |
| monoxygenase from | PSSYPTYPTKQQFVQYLESYAEHFDLKPVFNQTVEEAKFDRR |
| Arabidopsis thaliana | CGLWRVRTTGGKKDETMEYVSRWLVVATGENAEEVMPEID |
| SEQ ID NO: 108 | GIPDFGGPILHTSSYKSGEIFSEKKILVVGCGNSGMEVCLDLCN |
| | FNALPSLVVRDSVHVLPQEMLGISTFGISTSLLKWFPVHVVDR |
| | FLLRMSRLVLGDTDRLGLVRPKLGPLERKIKCGKTPVLDVGT |
| | LAKIRSGHIKVYPELKRVMHYSAEFVDGRVDNFDAIILATGY |
| | KSNVPMWLKGVNMFSEKDGFPHKPFPNGWKGESGLYAVGF |
| | TKLGLLGAAIDAKKIAEDIEVQRHFLPLARPQHC |
| IaaM: Tryptophan 2- | MYDHFNSPSIDILYDYGPFLKKCEMTGGIGSYSAGTPTPRVAI |
| monooxygenase from | VGAGISGLVAATELLRAGVKDVVLYESRDRIGGRVWSQVFD |
| Pseudomonas | QTRPRYIAEMGAMRFPPSATGLFHYLKKFGISTSTTFPDPGVV |
| savastanoi | DTELHYRGKRYHWPAGKKPPELFRRVYEGWQSLLSEGYLLE |
| SEQ ID NO: 109 | GGSLVAPLDITAMLKSGRLEEAAIAWQGWLNVFRDCSFYNAI |
| | VCIFTGRHPPGGDRWARPEDFELFGSLGIGSGGFLPVFQAGFT |
| | EILRMVINGYQSDQRLIPDGISSLAARLADQSFDGKALRDRVC |
| | FSRVGRISREAEKIIIQTEAGEQRVFDRVIVTSSNRAMQMIHCL |
| | TDSESFLSRDVARAVRETHLTGSSKLFILTRTKFWIKNKLPTTI |
| | QSDGLVRGVYCLDYQPDEPEGHGVVLLSYTWEDDAQKMLA |
| | MPDKKTRCQVLVDDLAAIHPTFASYLLPVDGDYERYVLHHD |
| | WLTDPHSAGAFKLNYPGEDVYSQRLFFQPMTANSPNKDTGL |
| | YLAGCSCSFAGGWIEGAVQTALNSACAVLRSTGGQLSKGNPL |
| | DCINASYRY |
| iaaH: | MHEIITLESLCQALADGEIAAAELRERALDTEARLARLNCFIRE |
| Indoleacetamide | GDAVSQFGEADHAMKGTPLWGMPVSFKDNICVRGLPLTAGT |
| hydrolase from | RGMSGFVSDQDAAIVSQLRALGAVVAGKNNMHELSFGVTSI |
| Pseudomonas | NPHWGTVGNPVAPGYCAGGSSGGSAAAVASGIVPLSVGTDT |
| savastanoi | GGSIRIPAAFCGITGFRPTTGRWSTAGIIPVSHTKDCVGLLTRT |
| SEQ ID NO: 110 | AGDAGFLYGLLSGKQQSFPLSRTAPCRIGLPVSMWSDLDGEV |
| | ERACVNALSLLRKTGFEFIEIDDADIVELNQTLTFTVPLYEFFA |
| | DLAQSLLSLGWKHGIHHIFAQVDDANVKGIINHHLGEGAIKP |
| | AHYLSSLQNGELLKRKMDELFARHNIELLGYPTVPCRVPHLD |
| | HADRPEFFSQAIRNTDLASNAMLPSITIPVGPEGRLPVGLSFDA |
| | LRGRDALLLSRVSAIEQVLGFVRKVLPHTT |
| TrpDH: Tryptophan | MLLFETVREMGHEQVLFCHSKNPEIKAIIAIHDTTLGPAMGAT |
| dehydrogenase from | RILPYINEEAALKDALRLSRGMTYKAACANIPAGGGKAVIIAN |
| Nostoc punctiforme | PENKTDDLLRAYGRFVDSLNGRFITGQDVNITPDDVRTISQET |
| NIES-2108 | KYVVGVSEKSGGPAPITSLGVFLGIKAAVESRWQSKRLDGMK |
| SEQ ID NO: 111 | VAVQGLGNVGKNLCRHLHEHDVQLFVSDVDPIKAEEVKRLF |
| | GATVVEPTEIYSLDVDIFAPCALGGILNSHTIPFLQASIIAGAAN |
| | NQLENEQLHSQMLAKKGILYSPDYVINAGGLINVYNEMIGYD |
| | EEKAFKQVHNIYDTLLAIFEIAKEQGVTTNDAARRLAEDRINN |
| | SKRSKSKAIAA |
| CYP79B2: | MNTFTSNSSDLTTTATETSSFSTLYLLSTLQAFVAITLVMLLKK |
| tryptophan N- | LMTDPNKKKPYLPPGPTGWPIIGMIPTMLKSRPVFRWLHSIMK |
| monooxygenase from | QLNTEIACVKLGNTHVITVTCPKIAREILKQQDALFASRPLTY |
| Arabidopsis thaliana | AQKILSNGYKTCVITPFGDQFKKMRKVVMTELVCPARHRWL |
| SEQ ID NO: 112 | HQKRSEENDHLTAWVYNMVKNSGSVDFRFMTRHYCGNAIK |
| | KLMFGTRTFSKNTAPDGGPTVEDVEHMEAMFEALGFTFAFCI |
| | SDYLPMLTGLDLNGHEKIMRESSAIMDKYHDPIIDERIKMWR |
| | EGKRTQIEDFLDIFISIKDEQGNPLLTADEIKPTIKELVMAAPDN |
| | PSNAVEWAMAEMVNKPEILRKAMEEIDRVVGKERLVQESDIP |
| | KLNYVKAILREAFRLHPVAAFNLPHVALSDTTVAGYHIPKGS |
| | QVLLSRYGLGRNPKVWADPLCFKPERHLNECSEVTLTENDLR |
| | FISFSTGKRGCAAPALGTALTTMMLARLLQGFTWKLPENETR |
| | VELMESSHDMFLAKPLVMVGDLRLPEHLYPTVK |
| CYP79B3: | MDTLASNSSDLTTKSSLGMSSFTNMYLLTTLQALAALCFLMI |
| tryptophan N- | LNKIKSSSRNKKLHPLPPGPTGFPIVGMIPAMLKNRPVFRWLH |
| monooxygenase from | SLMKELNTEIACVRLGNTHVIPVTCPKIAREIFKQQDALFASRP |
| Arabidopsis thaliana | LTYAQKILSNGYKTCVITPFGEQFKKMRKVIMTEIVCPARHR |
| SEQ ID NO: 113 | WLHDNRAEETDHLTAWLYNMVKNSEPVDLRFVTRHYCGNA |
| | IKRLMFGTRTFSEKTEADGGPTLEDIEHMDAMFEGLGFTFAFC |
| | ISDYLPMLTGLDLNGHEKIMRESSAIMDKYHDPIIDERIKMWR |
| | EGKRTQIEDFLDIFISIKDEAGQPLLTADEIKPTIKELVMAAPDN |
| | PSNAVEWAIAEMINKPEILHKAMEEIDRVVGKERFVQESDIPK |
| | LNYVKAIIREAFRLHPVAAFNLPHVALSDTTVAGYHIPKGSQV |
| | LLSRYGLGRNPKVWSDPLSFKPERHLNECSEVTLTENDLRFIS |
| | FSTGKRGCAAPALGTAITTMMLARLLQGFKWKLAGSETRVE |
| | LMESSHDMFLSKPLVLVGELRLSEDLYPMVK |
| CYP71A13: | MSNIQEMEMILSISLCLTTLITLLLLRRFLKRTATKVNLPPSPW |
| indoleacetaldoxime | RLPVIGNLHQLSLHPHRSLRSLSLRYGPLMLLHFGRVPILVVSS |
| dehydratase from | GEAAQEVLKTHDHKFANRPRSKAVHGLMNGGRDVVFAPYG |
| Arabidopis thaliana | EYWRQMKSVCILNLLTNKMVESFEKVREDEVNAMIEKLEKA |
| SEQ ID NO: 114 | SSSSSSENLSELFITLPSDVTSRVALGRKHSEDETARDLKKRVR |
| | QIMELLGEFPIGEYVPILAWIDGIRGFNNKIKEVSRGFSDLMDK |
| | VVQEHLEASNDKADFVDILLSIEKDKNSGFQVQRNDIKFMILD |
| | MFIGGTSTTSTLLEWTMTELIRSPKSMKKLQDEIRSTIRPHGSY |
| | IKEKEVENMKYLKAVIKEVLRLHPSLPMILPRLLSEDVKVKGY |
| | NIAAGTEVIINAWAIQRDTAIWGPDAEEFKPERHLDSGLDYHG |
| | KNLNYIPFGSGRRICPGINLALGLAEVTVANLVGRFDWRVEA |
| | GPNGDQPDLTEAIGIDVCRKFPLIAFPSSVV |
| PEN2: myrosinase | MAHLQRTFPTEMSKGRASFPKGFLFGTASSSYQYEGAVNEGA |
| from Arabidopsis | RGQSVWDHFSNRFPHRISDSSDGNVAVDFYHRYKEDIKRMK |
| thaliana | DINMDSFRLSIAWPRVLPYGKRDRGVSEEGIKFYNDVIDELLA |
| SEQ ID NO: 115 | NEITPLVTIFHWDIPQDLEDEYGGFLSEQIIDDFRDYASLCFERF |
| | GDRVSLWCTMNEPWVYSVAGYDTGRKAPGRCSKYVNGASV |
| | AGMSGYEAYIVSHNMLLAHAEAVEVFRKCDHIKNGQIGIAHN |
| | PLWYEPYDPSDPDDVEGCNRAMDFMLGWHQHPTACGDYPE |
| | TMKKSVGDRLPSFTPEQSKKLIGSCDYVGINYYSSLFVKSIKH |
| | VDPTQPTWRTDQGVDWMKTNIDGKQIAKQGGSEWSFTYPTG |
| | LRNILKYVKKTYGNPPILITENGYGEVAEQSQSLYMYNPSIDT |
| | ERLEYIEGHIHAIHQAIHEDGVRVEGYYVWSLLDNFEWNSGY |
| | GVRYGLYYIDYKDGLRRYPKMSALWLKEFLRFDQEDDSSTS |
| | KKEEKKESYGKQLLHSVQDSQFVHSIKDSGALPAVLGSLFVV |
| | SATVGTSLFFKGANN |
| Nit1: Nitrilase from | MSSTKDMSTVQNATPFNGVAPSTTVRVTIVQSSTVYNDTPATI |
| Arabidopsis thaliana | DKAEKYIVEAASKGAELVLFPEGFIGGYPRGFRFGLAVGVHN |
| SEQ ID NO: 116 | EEGRDEFRKYHASAIHVPGPEVARLADVARKNHVYLVMGAI |
| | EKEGYTLYCTVLFFSPQGQFLGKHRKLMPTSLERCIWGQGDG |
| | STIPVYDTPIGKLGAAICWENRMPLYRTALYAKGIELYCAPTA |
| | DGSKEWQSSMLHIAIEGGCFVLSACQFCQRKHFPDHPDYLFT |
| | DWYDDKEHDSIVSQGGSVIISPLGQVLAGPNFESEGLVTADID |
| | LGDIARAKLYFDSVGHYSRPDVLHLTVNEHPRKSVTFVTKVE |
| | KAEDDSNK |
| IDO1: indoleamine | MAHAMENSWTISKEYHIDEEVGFALPNPQENLPDFYNDWMFI |
| 2,3-dioxygenase from | AKHLPDLIESGQLRERVEKLNMLSIDHLTDHKSQRLARLVLG |
| homo sapiens | CITMAYVWGKGHGDVRKVLPRNIAVPYCQLSKKLELPPILVY |
| SEQ ID NO: 117 | ADCVLANWKKKDPNKPLTYENMDVLFSFRDGDCSKGFFLVS |
| | LLVEIAAASAIKVIPTVFKAMQMQERDTLLKALLEIASCLEKA |
| | LQVFHQIHDHVNPKAFFSVLRIYLSGWKGNPQLSDGLVYEGF |
| | WEDPKEFAGGSAGQSSVFQCFDVLLGIQQTAGGGHAAQFLQ |
| | DMRRYMPPAHRNFLCSLESNPSVREFVLSKGDAGLREAYDA |
| | CVKALVSLRSYHLQIVTKYILIPASQQPKENKTSEDPSKLEAK |
| | GTGGTDLMNFLKTVRSTTEKSLLKEG |
| TDO2: tryptophan | MSGCPFLGNNFGYTFKKLPVEGSEEDKSQTGVNRASKGGLIY |
| 2,3-dioxygenase from | GNYLHLEKVLNAQELQSETKGNKIHDEHLFIITHQAYELWFK |
| homo sapiens | QILWELDSVREIFQNGHVRDERNMLKVVSRMHRVSVILKLLV |
| SEQ ID NO: 118 | QQFSILETMTALDFNDFREYLSPASGFQSLQFRLLENKIGVLQ |
| | NMRVPYNRRHYRDNFKGEENELLLKSEQEKTLLELVEAWLE |
| | RTPGLEPHGFNFWGKLEKNITRGLEEEFIRIQAKEESEEKEEQV |
| | AEFQKQKEVLLSLFDEKRHEHLLSKGERRLSYRALQGALMIY |
| | FYREEPRFQVPFQLLTSLMDIDSLMTKWRYNHVCMVHRMLG |
| | SKAGTGGSSGYHYLRSTVSDRYKVFVDLFNLSTYLIPRHWIPK |
| | MNPTIHKFLYTAEYCDSSYFSSDESD |
| BNA2: indoleamine | MNNTSITGPQVLHRTKMRPLPVLEKYCISPHHGFLDDRLPLTR |
| 2,3-dioxygenase from | LSSKKYMKWEEIVADLPSLLQEDNKVRSVIDGLDVLDLDETIL |
| S. cerevisiae | GDVRELRRAYSILGFMAHAYIWASGTPRDVLPECIARPLLETA |
| SEQ ID NO: 119 | HILGVPPLATYSSLVLWNFKVTDECKKTETGCLDLENITTINTF |
| | TGTVDESWFYLVSVRFEKIGSACLNHGLQILRAIRSGDKGDA |
| | NVIDGLEGLAATIERLSKALMEMELKCEPNVFYFKIRPFLAGW |
| | TNMSHMGLPQGVRYGAEGQYRIFSGGSNAQSSLIQTLDILLG |
| | VKHTANAAHSSQGDSKINYLDEMKKYMPREHREFLYHLESV |
| | CNIREYVSRNASNRALQEAYGRCISMLKIFRDNHIQIVTKYIIL |
| | PSNSKQHGSNKPNVLSPIEPNTKASGCLGHKVASSKTIGTGGT |
| | RLMPFLKQCRDETVATADIKNEDKN |
| Afmid: Kynurenine | MAFPSLSAGQNPWRNLSSEELEKQYSPSRWVIHTKPEEVVGN |
| formamidase from | FVQIGSQATQKARATRRNQLDVPYGDGEGEKLDIYFPDEDSK |
| mouse | AFPLFLFLHGGYWQSGSKDDSAFMVNPLTAQGIVVVIVAYDI |
| SEQ ID NO: 120 | APKGTLDQMVDQVTRSVVFLQRRYPSNEGIYLCGHSAGAHL |
| | AAMVLLARWTKHGVTPNLQGFLLVSGIYDLEPLIATSQNDPL |
| | RMTLEDAQRNSPQRHLDVVPAQPVAPACPVLVLVGQHDSPE |
| | FHRQSKEFYETLLRVGWKASFQQLRGVDHFDIIENLTREDDV |
| | LTQIILKTVFQKL |
| BNA3: kynurenine-- | MKQRFIRQFTNLMSTSRPKVVANKYFTSNTAKDVWSLTNEA |
| oxoglutarate | AAKAANNSKNQGRELINLGQGFFSYSPPQFAIKEAQKALDIPM |
| transaminase from S. cerevisae | VNQYSPTRGRPSLINSLIKLYSPIYNTELKAENVTVTTGANEGI |
| SEQ ID NO: 121 | LSCLMGLLNAGDEVIVFEPFFDQYIPNIELCGGKVVYVPINPPK |
| | ELDQRNTRGEEWTIDFEQFEKAITSKTKAVIINTPHNPIGKVFT |
| | REELTTLGNICVKHNVVIISDEVYEHLYFTDSFTRIATLSPEIGQ |
| | LTLTVGSAGKSFAATGWRIGWVLSLNAELLSYAAKAHTRICF |
| | ASPSPLQEACANSINDALKIGYFEKMRQEYINKFKIFTSIFDEL |
| | GLPYTAPEGTYFVLVDFSKVKIPEDYPYPEEILNKGKDFRISH |
| | WLINELGVVAIPPTEFYIKEHEKAAENLLRFAVCKDDAYLEN |
| | AVERLKLLKDYL |
| GOT2: Aspartate | MALLHSGRVLPGIAAAFHPGLAAAASARASSWWTHVEMGPP |
| aminotransferase, | DPILGVTEAFKRDTNSKKMNLGVGAYRDDNGKPYVLPSVRK |
| mitochondrial from | AEAQIAAKNLDKEYLPIGGLAEFCKASAELALGENSEVLKSG |
| homo sapiens | RFVTVQTISGTGALRIGASFLQRFFKFSRDVFLPKPTWGNHTPI |
| SEQ ID NO: 122 | FRDAGMQLQGYRYYDPKTCGFDFTGAVEDISKIPEQSVLLLH |
| | ACAHNPTGVDPRPEQWKEIATVVKKRNLFAFFDMAYQGFAS |
| | GDGDKDAWAVRHFIEQGINVCLCQSYAKNMGLYGERVGAFT |
| | MVCKDADEAKRVESQLKILIRPMYSNPPLNGARIAAAILNTPD |
| | LRKQWLQEVKVMADRIIGMRTQLVSNLKKEGSTHNWQHITD |
| | QIGMFCFTGLKPEQVERLIKEFSIYMTKDGRISVAGVTSSNVG |
| | YLAHAIHQVTK |
| AADAT: | MNYARFITAASAARNPSPIRTMTDILSRGPKSMISLAGGLPNP |
| Kynurenine/alpha- | NMFPFKTAVITVENGKTIQFGEEMMKRALQYSPSAGIPELLSW |
| aminoadipate | LKQLQIKLHNPPTIHYPPSQGQMDLCVTSGSQQGLCKVFEMII |
| aminotransferase, | NPGDNVLLDEPAYSGTLQSLHPLGCNIINVASDESGIVPDSLR |
| mitochondrial | DILSRWKPEDAKNPQKNTPKFLYTVPNGNNPTGNSLTSERKK |
| SEQ ID NO: 123 | EIYELARKYDFLIIEDDPYYFLQFNKFRVPTFLSMDVDGRVIRA |
| | DSFSKIISSGLRIGFLTGPKPLIERVILHIQVSTLHPSTFNQLMIS |
| | QLLHEWGEEGFMAHVDRVIDFYSNQKDAILAAADKWLTGLA |
| | EWHVPAAGMFLWIKVKGINDVKELIEEKAVKMGVLMLPGN |
| | AFYVDSSAPSPYLRASFSSASPEQMDVAFQVLAQLIKESL |
| CCLB1: Kynurenine-- | MAKQLQARRLDGIDYNPWVEFVKLASEHDVVNLGQGFPDFP |
| oxoglutarate | PPDFAVEAFQHAVSGDFMLNQYTKTFGYPPLTKILASFFGELL |
| transaminase 1 from | GQEIDPLRNVLVTVGGYGALFTAFQALVDEGDEVIIIEPFFDC |
| homo sapiens | YEPMTMMAGGRPVFVSLKPGPIQNGELGSSSNWQLDPMELA |
| SEQ ID NO: 124 | GKFTSRTKALVLNTPNNPLGKVFSREELELVASLCQQHDVVCI |
| | TDEVYQWMVYDGHQHISIASLPGMWERTLTIGSAGKTFSATG |
| | WKVGWVLGPDHIMKHLRTVHQNSVFHCPTQSQAAVAESFER |
| | EQLLFRQPSSYFVQFPQAMQRCRDHMIRSLQSVGLKPIIPQGS |
| | YFLITDISDFKRKMPDLPGAVDEPYDRRFVKWMIKNKGLVAI |
| | PVSIFYSVPHQKHFDHYIRFCFVKDEATLQAMDEKLRKWKVEL |
| CCLB2: kynurenine-- | MFLAQRSLCSLSGRAKFLKTISSSKILGFSTSAKMSLKFTNAKR |
| oxoglutarate | IEGLDSNVWIEFTKLAADPSVVNLGQGFPDISPPTYVKEELSKI |
| transaminase 3 from | AAIDSLNQYTRGFGHPSLVKALSYLYEKLYQKQIDSNKEILVT |
| homo sapiens | VGAYGSLFNTIQALIDEGDEVILIVPFYDCYEPMVRMAGATPV |
| SEQ ID NO: 125 | FIPLRSKPVYGKRWSSSDWTLDPQELESKFNSKTKAIILNTPHN |
| | PLGKVYNREELQVIADLCIKYDTLCISDEVYEWLVYSGNKHL |
| | KIATFPGMWERTITIGSAGKTFSVTGWKLGWSIGPNHLIKHLQ |
| | TVQQNTIYTCATPLQEALAQAFWIDIKRMDDPECYFNSLPKEL |
| | EVKRDRMVRLLESVGLKPIVPDGGYFIIADVSLLDPDLSDMK |
| | NNEPYDYKFVKWMTKHKKLSAIPVSAFCNSETKSQFEKFVRF |
| | CFIKKDSTLDAAEEIIKAWSVQKS |
| TnaA: tryptophanase | MENFKHLPEPFRIRVIEPVKRTTRAYREEAIIKSGMNPFLLDSE |
| from E. coli | DVFIDLLTDSGTGAVTQSMQAAMMRGDEAYSGSRSYYALAE |
| SEQ ID NO: 126 | SVKNIFGYQYTIPTHQGRGAEQIYIPVLIKKREQEKGLDRSKM |
| | VAFSNYFFDTTQGHSQINGCTVRNVYIKEAFDTGVRYDFKGN |
| | FDLEGLERGIEEVGPNNVPYIVATITSNSAGGQPVSLANLKAM |
| | YSIAKKYDIPVVMDSARFAENAYFIKQREAEYKDWTIEQITRE |
| | TYKYADMLAMSAKKDAMVPMGGLLCMKDDSFFDVYTECRT |
| | LCVVQEGFPTYGGLEGGAMERLAVGLYDGMNLDWLAYRIA |
| | QVQYLVDGLEEIGVVCQQAGGHAAFVDAGKLLPHIPADQFP |
| | AQALACELYKVAGIRAVEIGSFLLGRDPKTGKQLPCPAELLRL |
| | TIPRATYTQTHMDFIIEAFKHVKENAANIKGLTFTYEPKVLRH |
| | FTAKLKEV |
| Trp | MTATTISIETVPQAPAAGTKTNGTSGKYNPRTYLSDRAKVTEI |
| aminotransferase | DGSDAGRPNPDTFPFNSITLNLKPPLGLPESSNNMPVSITIEDPD |
| (EC 2.6.1.27); | LATALQYAPSAGIPKLREWLADLQAHVHERPRGDYAISVGSG |
| tryptophan | SQDLMFKGFQAVLNPGDPVLLETPMYSGVLPALRILKADYAE |
| aminotransferase | VDVDDQGLSAKNLEKVLSEWPADKKRPRVLYTSPIGSNPSGC |
| [Cryptococcus | SASKERKLEVLKVCKKYDVLIFEDDPYYYLAQELIPSYFALEK |
| deuterogattii R265] | QVYPEGGHVVRFDSFSKLLSAGMRLGFATGPKEILHAIDVSTA |
| SEQ ID NO: 97 | GANLHTSAVSQGVALRLMQYWGIEGFLAHGRAVAKLYTERR |
| | AQFEATAHKYLDGLATWVSPVAGMFLWIDLRPAGIEDSYELI |
| | RHEALAKGVLGVPGMAFYPTGRKSSHVRVSFSIVDLEDESDL |
| | GFQRLAEAIKDKRKALGLA |
| Trp | ATGACGGCAACTACAATTTCTATTGAGACCGTACCTCAGGC |
| aminotransferase | CCCGGCGGCGGGGACCAAAACTAATGGGACTTCAGGAAAA |
| (EC 2.6.1.27); | TACAACCCCCGCACTTACCTGTCCGACCGCGCCAAAGTCAC |
| tryptophan | TGAGATTGATGGATCTGACGCCGGTCGCCCCAATCCCGATA |
| aminotransferase | CTTTCCCATTTAACTCGATTACCTTAAATTTGAAACCACCTT |
| [Cryptococcus | TAGGCTTGCCCGAGAGTTCAAATAACATGCCGGTCTCTATC |
| deuterogattii R265], | ACGATTGAAGACCCCGATTTAGCGACGGCCTTACAATATG |
| codon optimized for | CACCTAGCGCCGGTATTCCTAAGCTGCGCGAATGGCTGGCT |
| expression in E. coli | GACTTACAAGCTCACGTTCATGAGCGCCCCCGTGGCGATTA |
| SEQ ID NO: 98 | TGCCATCTCGGTCGGGTCGGGGTCACAGGATTTGATGTTTA |
| | AGGGCTTCCAAGCTGTCTTGAATCCAGGTGATCCAGTCCTT |
| | CTGGAAACCCCAATGTATTCAGGTGTTCTGCCAGCGCTGCG |
| | CATTCTGAAGGCGGATTATGCAGAAGTTGATGTAGACGAC |
| | CAGGGGTTATCTGCTAAAAACCTTGAAAAAGTTTTATCAGA |
| | GTGGCCCGCAGATAAGAAGCGTCCTCGTGTCCTGTATACGT |
| | CGCCAATCGGCTCCAATCCTTCCGGATGTTCAGCATCCAAG |
| | GAACGCAAGTTAGAGGTACTGAAAGTCTGTAAGAAGTACG |
| | ATGTGCTGATCTTCGAAGACGATCCGTATTATTACCTTGCT |
| | CAAGAGCTTATTCCATCCTATTTTGCGTTGGAAAAACAAGT |
| | TTATCCGGAGGGTGGGCACGTTGTACGCTTTGACTCATTTA |
| | GTAAATTGCTTTCTGCTGGGATGCGCTTGGGATTTGCTACA |
| | GGGCCGAAGGAAATTCTTCATGCGATTGACGTCAGTACAG |
| | CAGGCGCAAATTTACATACTTCAGCGGTCTCTCAAGGTGTC |
| | GCTCTTCGCCTGATGCAGTATTGGGGGATCGAGGGATTCCT |
| | TGCACATGGCCGCGCGGTGGCCAAACTTTACACGGAGCGC |
| | CGCGCTCAGTTCGAGGCAACCGCACATAAGTACCTGGACG |
| | GGCTGGCCACTTGGGTATCTCCCGTAGCGGGAATGTTTTTA |
| | TGGATCGATCTTCGTCCAGCAGGAATCGAAGATTCTTACGA |
| | ATTAATTCGCCATGAAGCATTAGCCAAAGGCGTTTTAGGCG |
| | TTCCAGGGATGGCGTTTTATCCGACAGGCCGTAAGTCTTCC |
| | CATGTTCGTGTCAGTTTCAGTATCGTCGACCTGGAAGACGA |
| | ATCTGACCTTGGTTTTCAACGCCTGGCTGAAGCTATTAAGG |
| | ATAAACGCAAGGCTTTAGGGCTGGCT |
| Tryptophan | MSQVIKKKRNTFMIGTEYILNSTQLEEAIKSFVHDFCAEKHEIH |
| Decarboxylase (EC | DQPVVVEAKEHQEDKIKQIKIPEKGRPVNEVVSEMMNEVYRY |
| 4.1.1.28) Chain A, | RGDANHPRFFSFVPGPASSVSWLGDIMTSAYNIHAGGSKLAP |
| Ruminococcus | MVNCIEQEVLKWLAKQVGFTENPGGVFVSGGSMANITALTA |
| Gnavus Tryptophan | ARDNKLTDINLHLGTAYISDQTHSSVAKGLRIIGITDSRIRRIPT |
| | NSHFQMDTTKLEEAIETDKKSGYIPFVVIGTAGTTNTGSIDPLT |
| | EISALCKKHDMWFHIDGAYGASVLLSPKYKSLLTGTGLADSIS |
| | WDAHKWLFQTYGCAMVLVKDIRNLFHSFHVNPEYLKDLEN |
| | DIDNVNTWDIGMELTRPARGLKLWLTLQVLGSDLIGSAIEHG |
| | FQLAVWAEEALNPKKDWEIVSPAQMAMINFRYAPKDLTKEE |
| | QDILNEKISHRILESGYAAIFTTVLNGKTVLRICAIHPEATQED |
| | MQHTIDLLDQYGREIYTEMKKa |
| Tryptophan | ATGAGTCAAGTGATTAAGAAGAAACGTAACACCTTTATGA |
| Decarboxylase (EC | TCGGAACGGAGTACATTCTTAACAGTACACAATTGGAGGA |
| 4.1.1.28) Chain A, | AGCGATTAAATCATTCGTACATGATTTCTGCGCAGAGAAGC |
| Ruminococcus | ATGAGATCCATGATCAACCTGTGGTAGTAGAAGCTAAAGA |
| Gnavus Tryptophan | ACATCAGGAGGACAAAATCAAACAAATCAAAATCCCGGAA |
| Decarboxylase Rumgna_01526 | AAGGGACGTCCTGTAAATGAAGTCGTTTCTGAGATGATGA |
| (alpha- | ATGAAGTGTATCGCTACCGCGGAGACGCCAACCATCCTCG |
| fmt); codon | CTTTTTTTCTTTTGTGCCCGGACCTGCAAGCAGTGTGTCGTG |
| optimized for the | GTTGGGGGATATTATGACGTCCGCCTACAATATTCATGCTG |
| expression in E. coli | GAGGCTCAAAGCTGGCACCGATGGTTAACTGCATTGAGCA |
| SEQ ID NO: 96 | GGAAGTTCTGAAGTGGTTAGCAAAGCAAGTGGGGTTCACA |
| | GAAAATCCAGGTGGCGTATTTGTGTCGGGCGGTTCAATGG |
| | CGAATATTACGGCACTTACTGCGGCTCGTGACAATAAACTG |
| | ACCGACATTAACCTTCATTTGGGAACTGCTTATATTAGTGA |
| | CCAGACTCATAGTTCAGTTGCGAAAGGATTACGCATTATTG |
| | GAATCACTGACAGTCGCATCCGTCGCATTCCCACTAACTCC |
| | CACTTCCAGATGGATACCACCAAGCTGGAGGAAGCCATCG |
| | AGACCGACAAGAAGTCTGGCTACATTCCGTTCGTCGTTATC |
| | GGAACAGCAGGTACCACCAACACTGGTTCGATTGACCCCC |
| | TGACAGAAATCTCTGCGTTATGTAAGAAGCATGACATGTG |
| | GTTTCATATCGACGGAGCGTATGGAGCTAGTGTTCTGCTGT |
| | CACCTAAGTACAAGAGCCTTCTTACCGGAACCGGCTTGGCT |
| | GACAGTATTTCGTGGGATGCTCATAAATGGTTGTTCCAAAC |
| | GTACGGCTGTGCAATGGTACTTGTCAAAGATATCCGTAATT |
| | TATTCCACTCTTTTCATGTGAATCCCGAGTATCTTAAGGAT |
| | CTGGAAAACGACATCGATAACGTTAATACATGGGACATCG |
| | GCATGGAGCTGACGCGCCCTGCACGCGGTCTTAAATTGTG |
| | GCTTACTTTACAGGTCCTTGGATCTGACTTGATTGGGAGTG |
| | CCATTGAACACGGTTTCCAGCTGGCAGTTTGGGCTGAGGA |
| | AGCATTGAATCCAAAGAAAGACTGGGAGATCGTTTCTCCA |
| | GCTCAGATGGCTATGATTAATTTCCGTTATGCCCCTAAGGA |
| | TTTAACCAAAGAGGAACAGGATATTCTGAATGAAAAGATC |
| | TCCCACCGCATTTTAGAGAGCGGATACGCTGCAATTTTCAC |
| | TACTGTATTAAACGGCAAGACCGTTTTACGCATCTGTGCAA |
| | TTCACCCGGAGGCAACTCAAGAGGATATGCAACACACAAT |
| | CGACTTATTAGACCAATACGGTCGTGAAATCTATACCGAG |
| | ATGAAGAAAGCG |

In some embodiments, the genetically engineered bacteria comprise one or more nucleic acid sequence of Table 13 or a functional fragment thereof. In some embodiments, the genetically engineered bacteria comprise a nucleic acid sequence that, but for the redundancy of the genetic code, encodes the same polypeptide as one or more nucleic acid sequence of Table 13 or a functional fragment thereof. In some embodiments, genetically engineered bacteria comprise a nucleic acid sequence that is at least about 80%, at least about 85%, at least about 90%, at least about 95%, or at least about 99% homologous to the DNA sequence of one or more nucleic acid sequence of Table 13 or a functional fragment thereof, or a nucleic acid sequence that, but for the redundancy of the genetic code, encodes the same polypeptide as one or more nucleic acid sequence of Table 13 or a functional fragment thereof.

In one embodiment, the Tryptophan Decarboxylase gene has at least about 80% identity with the entire sequence of SEQ ID NO: 95 or SEQ ID NO: 96. In another embodiment, the Tryptophan Decarboxylase gene has at least about 85% identity with the entire sequence of SEQ ID NO: 95 or SEQ ID NO: 96. In one embodiment, the Tryptophan Decarboxylase gene has at least about 90% identity with the entire sequence of SEQ ID NO: 95 or SEQ ID NO: 96. In one embodiment, the Tryptophan Decarboxylase gene has at least about 95% identity with the entire sequence of SEQ ID NO: 95 or SEQ ID NO: 96. In another embodiment, the Tryptophan Decarboxylase gene has at least about 96%, 97%, 98%, or 99% identity with the entire sequence of SEQ ID NO: 95 or SEQ ID NO: 96. Accordingly, in one embodiment, the Tryptophan Decarboxylase gene has at least about 80%, 81%, 82%, 83%, 84%, 85%, 86%, 87%, 88%, 89%, 90%, 91%, 92%, 93%, 94%, 95%, 96%, 97%, 98%, or 99% identity with the entire sequence of SEQ ID NO: 95 or SEQ ID NO: 96. In another embodiment, the Tryptophan Decarboxylase gene comprises the sequence of SEQ ID NO: 95 or SEQ ID NO: 96. In yet another embodiment the Tryptophan Decarboxylase gene consists of the sequence of SEQ ID NO: 95 or SEQ ID NO: 96.

In some embodiments, the genetically engineered bacteria comprise one or more nucleic acid sequence of Table 14 or a functional fragment thereof. In some embodiments, the genetically engineered bacteria comprise a nucleic acid sequence that, but for the redundancy of the genetic code, encodes the same polypeptide as one or more nucleic acid sequence of Table 14 or a functional fragment thereof. In some embodiments, genetically engineered bacteria comprise a nucleic acid sequence that is at least about 80%, at least about 85%, at least about 90%, at least about 95%, or at least about 99% homologous to the DNA sequence of one or more nucleic acid sequence of Table 14 or a functional fragment thereof, or a nucleic acid sequence that, but for the redundancy of the genetic code, encodes the same polypeptide as one or more nucleic acid sequence of Table 14 or a functional fragment thereof.

In one embodiment, the Trp aminotransferase gene has at least about 80% identity with the entire sequence of SEQ ID NO: 97 or SEQ ID NO: 98. In another embodiment, the Trp aminotransferase gene has at least about 85% identity with the entire sequence of SEQ ID NO: 97 or SEQ ID NO: 98. In one embodiment, the Trp aminotransferase gene has at least about 90% identity with the entire sequence of SEQ ID NO: 97 or SEQ ID NO: 98. In one embodiment, the Trp aminotransferase gene has at least about 95% identity with the entire sequence of SEQ ID NO: 97 or SEQ ID NO: 98. In another embodiment, the Trp aminotransferase gene has at least about 96%, 97%, 98%, or 99% identity with the entire sequence of SEQ ID NO: 97 or SEQ ID NO: 98. Accordingly, in one embodiment, the Trp aminotransferase gene has at least about 80%, 81%, 82%, 83%, 84%, 85%, 86%, 87%, 88%, 89%, 90%, 91%, 92%, 93%, 94%, 95%, 96%, 97%, 98%, or 99% identity with the entire sequence of SEQ ID NO: 97 or SEQ ID NO: 98. In another embodiment, the Trp aminotransferase gene comprises the sequence of SEQ ID NO: 97 or SEQ ID NO: 98. In yet another embodiment the Trp aminotransferase gene consists of the sequence of SEQ ID NO: 97 or SEQ ID NO: 98.

In one embodiment, the tryptophan pathway catabolic enzyme has at least about 80% identity with the entire sequence of one or more of SEQ ID NO: 99 through SEQ ID NO: 126. In another embodiment, the tryptophan pathway catabolic enzyme has at least about 85% identity with the entire sequence of one or more SEQ ID NO: 99 through SEQ ID NO: 126. In one embodiment, the tryptophan pathway catabolic enzyme has at least about 90% identity with the entire sequence of one or more SEQ ID NO: 99 through SEQ ID NO: 126. In one embodiment, the tryptophan pathway catabolic enzyme has at least about 95% identity with the entire sequence of one or more SEQ ID NO: 99 through SEQ ID NO: 126. In another embodiment, the tryptophan pathway catabolic enzyme has at least about 96%, 97%, 98%, or 99% identity with the entire sequence of one or more SEQ ID NO: 99 through SEQ ID NO: 126. Accordingly, in one embodiment, the tryptophan pathway catabolic enzyme has at least about 80%, 81%, 82%, 83%, 84%, 85%, 86%, 87%, 88%, 89%, 90%, 91%, 92%, 93%, 94%, 95%, 96%, 97%, 98%, or 99% identity with the entire sequence of one or more SEQ ID NO: 99 through SEQ ID NO: 126. In another embodiment, the tryptophan pathway catabolic enzyme comprises the sequence of one or more SEQ ID NO: 99 through SEQ ID NO: 126. In yet another embodiment the tryptophan pathway catabolic enzyme consists of the sequence of one or more SEQ ID NO: 99 through SEQ ID NO: 126.

In some embodiments, the genetically engineered bacteria comprise a gene cassette for the production of tryptamine from tryptophan. In some embodiments, the genetically engineered bacteria take up tryptophan through an endogenous or exogenous transporter as described above herein. In some embodiments the bacteria further produce tryptamine from tryptophan. In some embodiments, the genetically engineered bacteria optionally comprise a tryptamine exporter. In some embodiments the genetically engineered bacteria comprise an exporter of one or more indole metabolites, in order to increase the export of indole metabolites produced.

Table 16 depicts non-limiting examples of contemplated polypeptide sequences, which are encoded by indole-3-propionate producing bacteria.

TABLE 16
Non-limiting Examples of Sequences for
indole-3-propionate Production

| Description | Sequence |
|---|---|
| FldA: indole-3- | MENNTNMFSGVKVIELANFIAAPAAGRFFADGGAEVIKIESPA |
| propionyl- | GDPLRYTAPSEGRPLSQEENTTYDLENANKKAIVLNLKSEKGK |
| CoA:indole-3- | KILHEMLAEADILLTNWRTKALVKQGLDYETLKEKYPKLVFA |
| lactate CoA | QITGYGEKGPDKDLPGFDYTAFFARGGVSGTLYEKGTVPPNV |
| transferase from | VPGLGDHQAGMFLAAGMAGALYKAKTTGQGDKVTVSLMHS |
| Clostridium | AMYGLGIMIQAAQYKDHGLVYPINRNETPNPFIVSYKSKDDYF |
| sporogenes | VQVCMPPYDVFYDRFMTALGREDLVGDERYNKIENLKDGRA |
| SEQ ID NO: 127 | KEVYSIIEQQMVTKTKDEWDKIFRDADIPFAIAQTWEDLLEDE |
| | QAWANDYLYKMKYPTGNERALVRLPVFFKEAGLPEYNQSPQI |
| | AENTVEVLKEMGYTEQEIEELEKDKDIMVRKEK |
| FldB: subunit of | MSDRNKEVKEKKAKHYLREITAKHYKEALEAKERGEKVGWC |
| indole-3-lactate | ASNFPQEIATTLGVKVVYPENHAAAVAARGNGQNMCEHAEA |
| dehydratase from | MGFSNDVCGYARVNLAVMDIGHSEDQPIPMPDFVLCCNNICN |
| Clostridium | QMIKWYEHIAKTLDIPMILIDIPYNTENTVSQDRIKYIRAQFDD |
| sporogenes | AIKQLEEITGKKWDENKFEEVMKISQESAKQWLRAASYAKYK |
| SEQ ID NO: 128 | PSPFSGFDLFNHMAVAVCARGTQEAADAFKMLADEYEENVKT |
| | GKSTYRGEEKQRILFEGIACWPYLRHKLTKLSEYGMNVTATV |
| | YAEAFGVIYENMDELMAAYNKVPNSISFENALKMRLNAVTST |
| | NTEGAVIHINRSCKLWSGFLYELARRLEKETGIPVVSFDGDQA |
| | DPRNFSEAQYDTRIQGLNEVMVAKKEAE |
| FldC: subunit of | MSNSDKFFNDFKDIVENPKKYIMKHMEQTGQKAIGCMPLYTP |
| indole-3-lactate | EELVLAAGMFPVGVWGSNTELSKAKTYFPAFICSILQTTLENA |
| dehydratase from | LNGEYDMLSGMMITNYCDSLKCMGQNFKLTVENIEFIPVTVPQ |
| Clostridium | NRKMEAGKEFLKSQYKMNIEQLEKISGNKITDESLEKAIEIYDE |
| sporogenes | HRKVMNDFSMLASKYPGIITPTKRNYVMKSAYYMDKKEHTE |
| SEQ ID NO: 129 | KVRQLMDEIKAIEPKPFEGKRVITTGIIADSEDLLKILEENNIAIV |
| | GDDIAHESRQYRTLTPEANTPMDRLAEQFANRECSTLYDPEKK |
| | RGQYIVEMAKERKADGIIFFMTKFCDPEEYDYPQMKKDFEEA |
| | GIPHVLIETDMQMKNYEQARTAIQAFSETL |
| FldD: indole-3- | MFFTEQHELIRKLARDFAEQEIEPIADEVDKTAEFPKEIVKKMA |
| acrylyl-CoA | QNGFFGIKMPKEYGGAGADNRAYVTIMEEISRASGVAGIYLSS |
| reductase from | PNSLLGTPFLLVGTDEQKEKYLKPMIRGEKTLAFALTEPGAGS |
| Clostridium | DAGALATTAREEGDYYILNGRKTFITGAPISDNIIVFAKTDMSK |
| sporogenes | GTKGITTFIVDSKQEGVSFGKPEDKMGMIGCPTSDIILENVKVH |
| SEQ ID NO: 130 | KSDILGEVNKGFITAMKTLSVGRIGVASQALGIAQAAVDEAVK |
| | YAKQRKQFNRPIAKFQAIQFKLANMETKLNAAKLLVYNAAYK |
| | MDCGEKADKEASMAKYFAAESAIQIVNDALQIHGGYGYIKDY |
| | KIERLYRDVRVIAIYEGTSEVQQMVIASNLLK |
| FldH1: indole-3- | MKILAYCVRPDEVDSFKKFSEKYGHTVDLIPDSFGPNVAHLAK |
| lactate | GYDGISILGNDTCNREALEKIKDCGIKYLATRTAGVNNIDFDA |
| dehydrogenase | AKEFGINVANVPAYSPNSVSEFTIGLALSLTRKIPFALKRVELN |
| from Clostridium | NFALGGLIGVELRNLTLGVIGTGRIGLKVIEGFSGFGMKKMIGY |
| sporogenes | DIFENEEAKKYIEYKSLDEVFKEADIITLHAPLTDDNYHMIGKE |
| SEQ ID NO: 131 | SIAKMKDGVFIINAARGALIDSEALIEGLKSGKIAGAALDSYEY |
| | EQGVFHNNKMNEIMQDDTLERLKSFPNVVITPHLGFYTDEAVS |
| | NMVEITLMNLQEFELKGTCKNQRVCK |
| FldH2: indole-3- | MKILMYSVREHEKPAIKKWLEANPGVQIDLCNNALSEDTVCK |
| lactate | AKEYDGIAIQQTNSIGGKAVYSTLKEYGIKQIASRTAGVDMIDL |
| dehydrogenase | KMASDSNILVTNVPAYSPNAIAELAVTHTMNLLRNIKTLNKRI |
| from Clostridium | AYGDYRWSADLIAREVRSVTVGVVGTGKIGRTSAKLFKGLGA |
| sporogenes | NVIGYDAYPDKKLEENNLLTYKESLEDLLREADVVTLHTPLLE |
| SEQ ID NO: 132 | STKYMINKNNLKYMKPDAFIVNTGRGGIINTEDLIEALEQNKIA |
| | GAALDTFENEGLFLNKVVDPTKLPDSQLDKLLKMDQVLITHH |
| | VGFFTTTAVQNIVDTSLDSVVEVLKTNNSVNKVN |
| AcuI: acrylyl- | MRAVLIEKSDDTQSVSVTELAEDQLPEGDVLVDVAYSTLNYK |
| CoA reductase | DALAITGKAPVVRRFPMVPGIDFTGTVAQSSHADFKPGDRVIL |
| from Rhodobacter | NGWGVGEKHWGGLAERARVRGDWLVPLPAPLDLRQAAMIG |
| sphaeroides | TAGYTAMLCVLALERHGVVPGNGEIVVSGAAGGVGSVATTLL |
| SEQ ID NO: 133 | AAKGYEVAAVTGRASEAEYLRGLGAASVIDRNELTGKVRPLG |
| | QERWAGGIDVAGSTVLANMLSMMKYRGVVAACGLAAGMDL |
| | PASVAPFILRGMTLAGVDSVMCPKTDRLAAWARLASDLDPAK |
| | LEEMTTELPFSEVIETAPKFLDGTVRGRIVIPVTP |

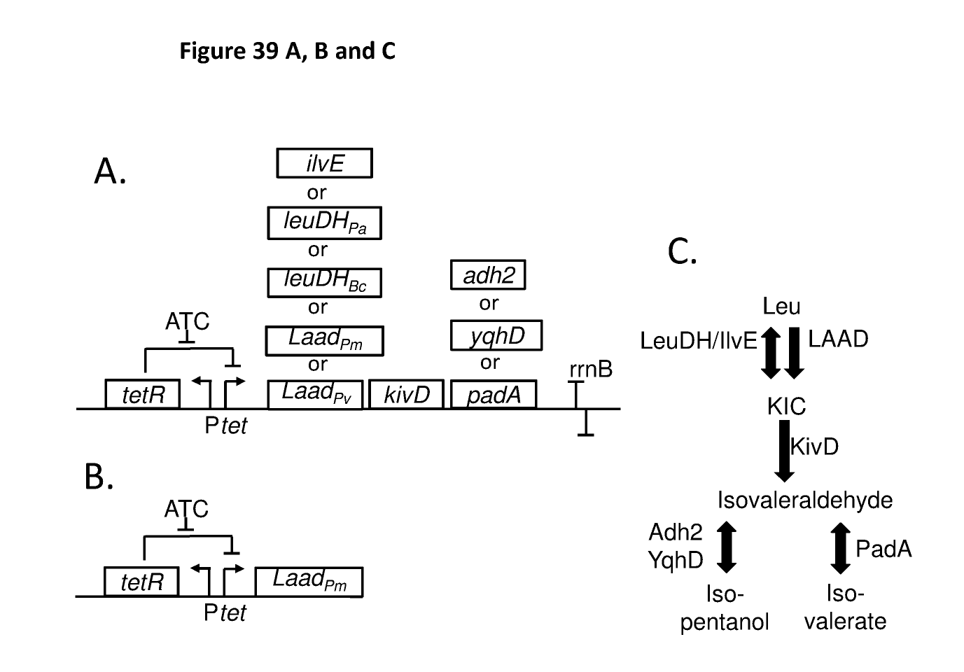

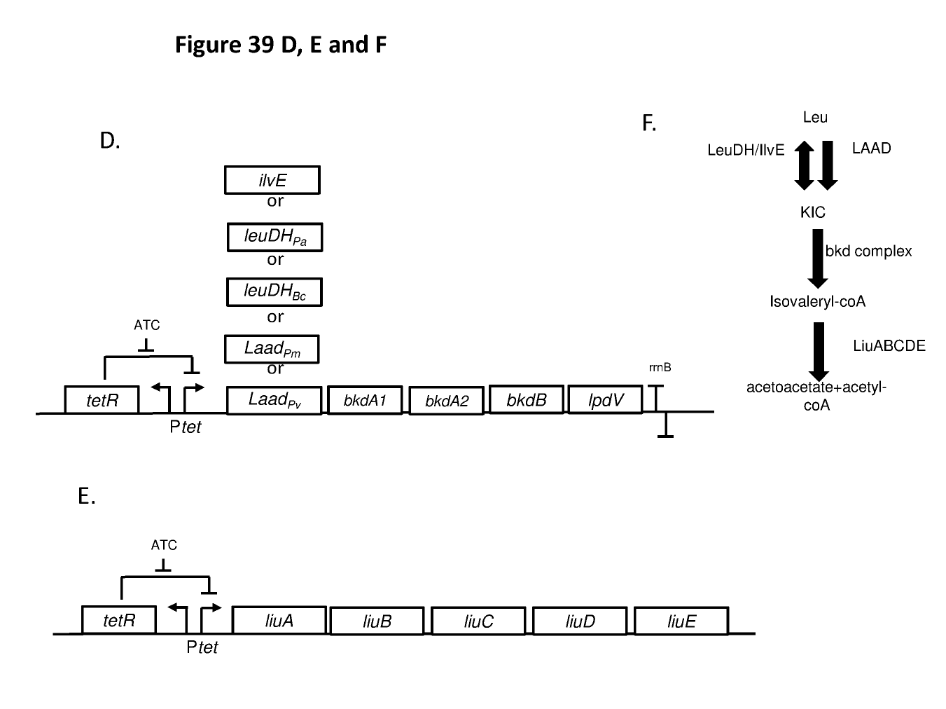

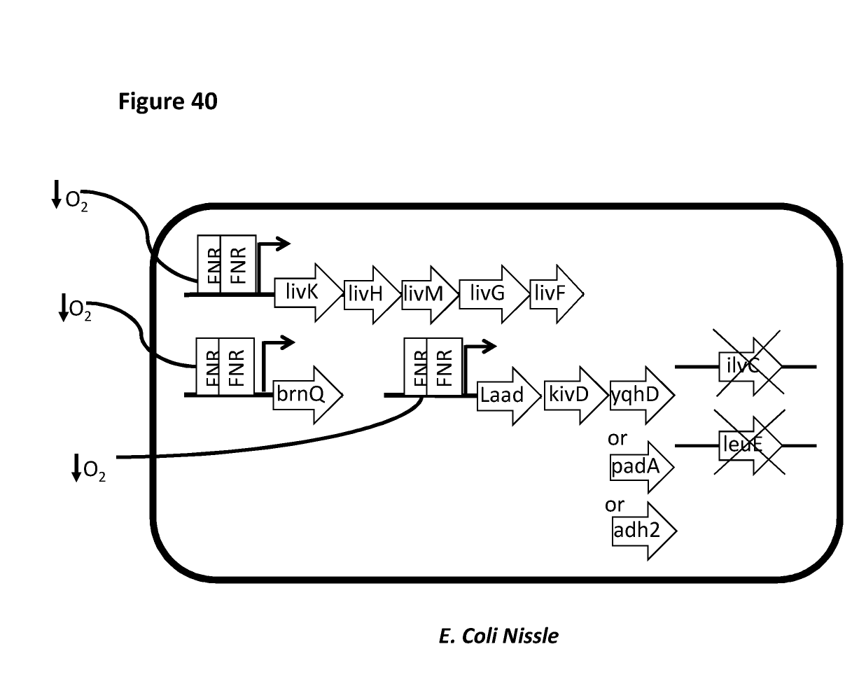

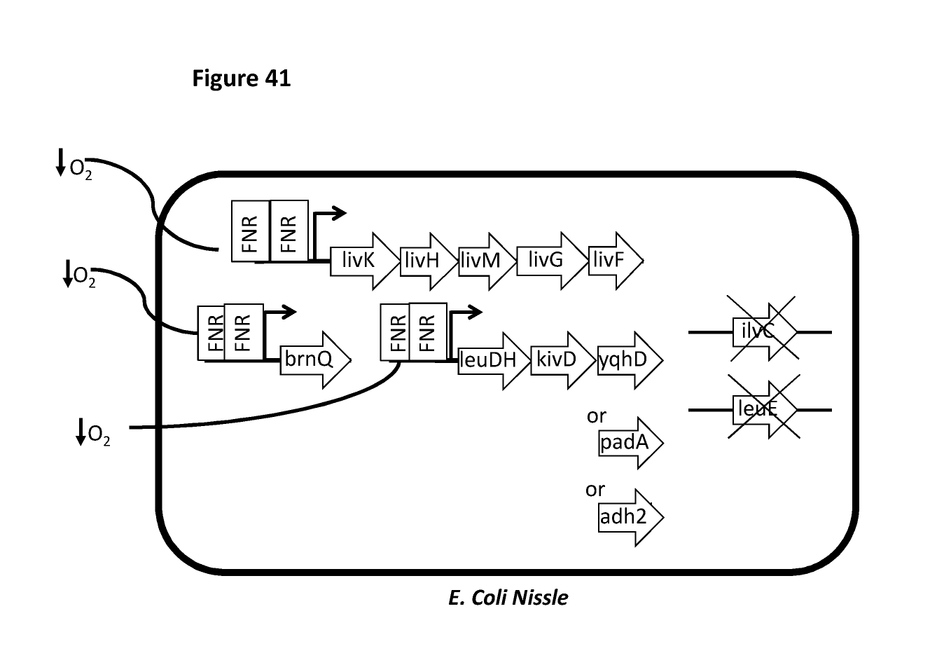

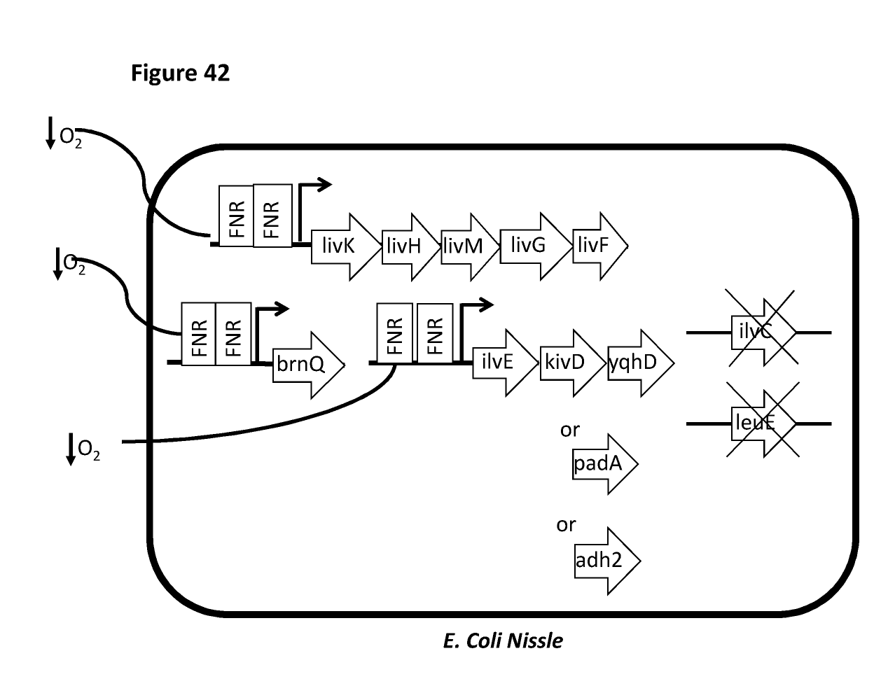

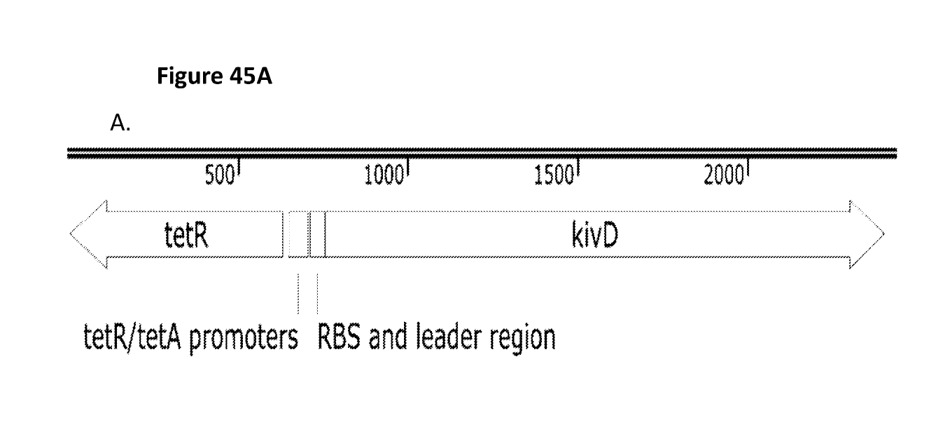

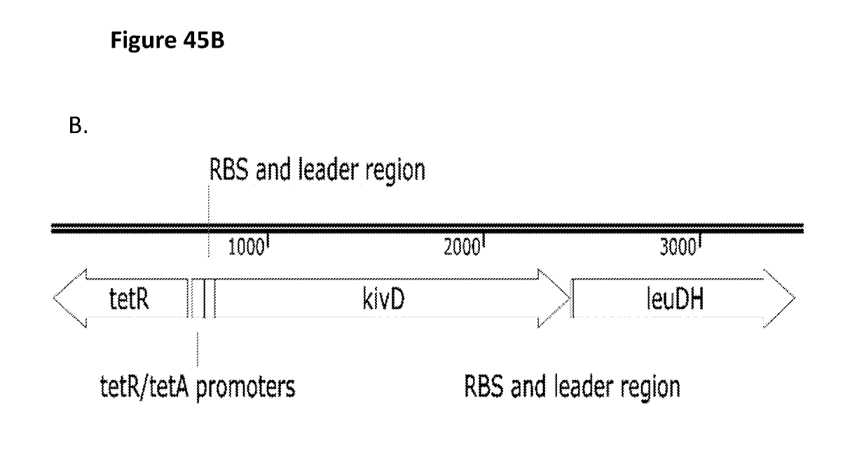

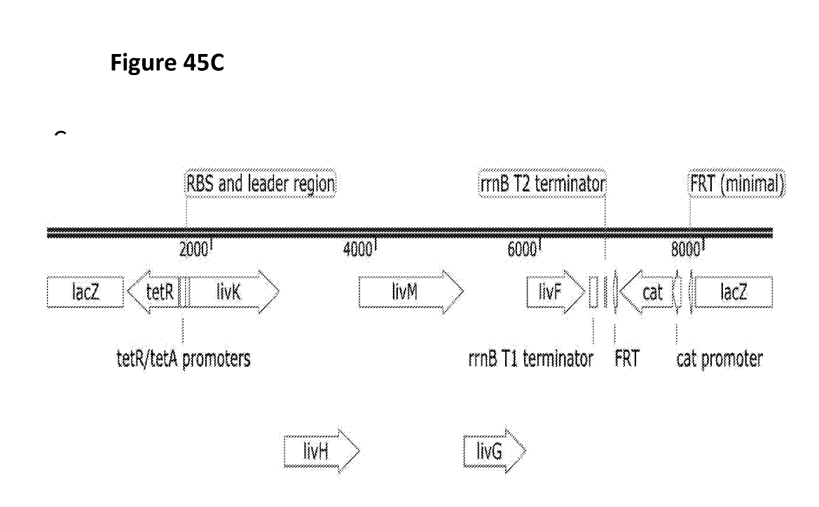

In some embodiments, the genetically engineered bacteria comprise a gene cassette for the production of one or more indole pathway metabolites described herein from tryptophan or a tryptophan metabolite. In some embodiments, the genetically engineered bacteria take up tryptophan through an endogenous or exogenous transporter as described above herein. In some embodiments, the genetically engineered bacteria additionally produce tryptophan and/or chorismate through any of the pathways described herein, e.g. FIG. 39, FIG. 45A and FIG. 45B. In some embodiments the genetically engineered bacteria comprise an exporter of one or more indole metabolites, in order to increase the export of indole metabolites produced.

In some embodiments, the genetically engineered bacteria are capable of expressing any one or more of the described circuits in low-oxygen conditions, in the presence of disease or tissue specific molecules or metabolites, in the presence of molecules or metabolites associated with inflammation or an inflammatory response or immune suppression or in the presence of some other metabolite that may or may not be present in the gut, such as arabinose or tetracycline. In some embodiments, any one or more of the described circuits are present on one or more plasmids (e.g., high copy or low copy) or are integrated into one or more sites in the bacterial chromosome. In some embodiments, the tryptophan synthesis and/or tryptophan catabolism cassette(s) is under control of an inducible promoter. Exemplary inducible promoters which may control the expression of the at least one sequence(s) include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline.

Also, in some embodiments, the genetically engineered bacteria are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more exporters for exporting biological molecules or substrates, such any of the exporters described herein or otherwise known in the art, (6) one or more secretion circuits, such as any of the secretion circuits described herein and otherwise known in the art, and (7) combinations of one or more of such additional circuits.

a. Tryptophan Repressor (TrpR)

In any of these embodiments, the tryptophan repressor (trpR) optionally may be deleted, mutated, or modified so as to diminish or obliterate its repressor function. Also, in any of these embodiments, the genetically engineered bacteria optionally comprise gene sequence(s) to produce the tryptophan precursor, Chorismate, e.g., sequence(s) encoding aroG, aroF, aroH, aroB, aroD, aroE, aroK, and AroC.

b. Tryptophan and Tryptophan Metabolite Transport

Metabolite transporters may further be expressed or modified in the genetically engineered bacteria of the invention in order to enhance tryptophan or KP metabolite transport into the cell.

The inner membrane protein YddG of E. coli, encoded by the yddG gene, is a homologue of the known amino acid exporters RhtA and YdeD. Studies have shown that YddG is capable of exporting aromatic amino acids, including tryptophan. Thus, YddG can function as a tryptophan exporter or a tryptophan secretion system (or tryptophan secretion protein). Other aromatic amino acid exporters are described in Doroshenko et al., FEMS Microbiol. Lett., 275:312-318 (2007). Thus, in some embodiments, the engineered bacteria optionally further comprise gene sequence(s) encoding YddG. In some embodiments, the engineered bacteria can over-express YddG. In some embodiments, the engineered bacteria optionally comprise one or more copies of yddG gene.

In some embodiments, the engineered microbe has a mechanism for importing (transporting) Kynurenine from the local environment into the cell. Thus, in some embodiments, the genetically engineered bacteria comprise gene sequence(s) encoding a kynureninase secreter. In some embodiments, the genetically engineered bacteria comprise one or more copies of aroP, tnaB or mtr gene.

In some embodiments the genetically engineered bacteria comprise a transporter to facilitate uptake of tryptophan into the cell. Three permeases, Mtr, TnaB, and AroP, are involved in the uptake of L-tryptophan in Escherichia coli. In some embodiments, the genetically engineered bacteria comprise one or more copies of one or more of Mtr, TnaB, and AroP.

In some embodiments, the genetically engineered bacteria of the invention also comprise multiple copies of the the transporter gene. In some embodiments, the genetically engineered bacteria of the invention also comprise a transporter gene from a different bacterial species. In some embodiments, the genetically engineered bacteria of the invention comprise multiple copies of a transporter gene from a different bacterial species. In some embodiments, the native transporter gene in the genetically engineered bacteria of the invention is not modified. In some embodiments, the genetically engineered bacteria of the invention comprise a transporter gene that is controlled by its native promoter, an inducible promoter, or a promoter that is stronger than the native promoter, e.g., a GlnRS promoter, a P(Bla) promoter, or a constitutive promoter.

In some embodiments, the native transporter gene in the genetically engineered bacteria is not modified, and one or more additional copies of the native transporter gene are inserted into the genome under the control of the same inducible promoter that controls expression of the payload, e.g., a FNR promoter, or a different inducible promoter than the one that controls expression of the payload or a constitutive promoter. In alternate embodiments, the native transporter gene is not modified, and a copy of a non-native transporter gene from a different bacterial species is inserted into the genome under the control of the same inducible promoter that controls expression of the payload, e.g., a FNR promoter, or a different inducible promoter than the one that controls expression of the payload or a constitutive promoter.

In some embodiments, the native transporter gene in the genetically engineered bacteria is not modified, and one or more additional copies of the native transporter gene are present in the bacteria on a plasmid and under the control of the same inducible promoter that controls expression of the payload e.g., a FNR promoter, or a different inducible promoter than the one that controls expression of the payload or a constitutive promoter. In alternate embodiments, the native transporter gene is not modified, and a copy of a non-native transporter gene from a different bacterial species is present in the bacteria on a plasmid and under the control of the same inducible promoter that controls expression of the payload, e.g., a FNR promoter, or a different inducible promoter than the one that controls expression of the payload or a constitutive promoter.

In some embodiments, the native transporter gene is mutagenized, the mutants exhibiting increased ammonia transport are selected, and the mutagenized transporter gene is isolated and inserted into the genetically engineered bacteria. In some embodiments, the native transporter gene is mutagenized, mutants exhibiting increased ammonia transport are selected, and those mutants are used to produce the bacteria of the invention. The transporter modifications described herein may be present on a plasmid or chromosome.

In some embodiments, the genetically engineered bacterium is E. coli Nissle, and the native transporter gene in E. coli Nissle is not modified; one or more additional copies the native E. coli Nissle transporter genes are inserted into the E. coli Nissle genome under the control of the same inducible promoter that controls expression of the payload e.g., a FNR promoter, or a different inducible promoter than the one that controls expression of the payload or a constitutive promoter. In an alternate embodiment, the native transporter gene in E. coli Nissle is not modified, and a copy of a non-native transporter gene from a different bacterium, e.g., Lactobacillus plantarum, is inserted into the E. coli Nissle genome under the control of the same inducible promoter that controls expression of the payload, e.g., a FNR promoter, or a different inducible promoter than the one that controls expression of the payload or a constitutive promoter.

In some embodiments, the genetically engineered bacterium is E. coli Nissle, and the native transporter gene in E. coli Nissle is not modified; one or more additional copies the native E. coli Nissle transporter genes are present in the bacterium on a plasmid and under the control of the same inducible promoter that controls expression of the payload, e.g., a FNR promoter, or a different inducible promoter than the one that controls expression of the payload, or a constitutive promoter. In an alternate embodiment, the native transporter gene in E. coli Nissle is not modified, and a copy of a non-native transporter gene from a different bacterium, e.g., Lactobacillus plantarum, are present in the bacterium on a plasmid and under the control of the same inducible promoter that controls expression of the payload, e.g., a FNR promoter, or a different inducible promoter than the one that controls expression of the payload, or a constitutive promoter.

Inducible Promoters

In some embodiments, the bacterial cell comprises a stably maintained plasmid or chromosome carrying the gene(s) encoding the amino acid catabolism enzyme(s), such that the amino acid catabolism enzyme(s) can be expressed in the host cell, and the host cell is capable of survival and/or growth in vitro, e.g., in medium, and/or in vivo, e.g., in the gut. In some embodiments, bacterial cell comprises two or more distinct amino acid catabolism enzymes or operons, e.g., two or more amino acid catabolism enzyme genes. In some embodiments, bacterial cell comprises three or more distinct amino acid catabolism enzymes or operons, e.g., three or more amino acid catabolism enzyme genes. In some embodiments, bacterial cell comprises 4, 5, 6, 7, 8, 9, 10, or more distinct amino acid catabolism enzymes or operons, e.g., 4, 5, 6, 7, 8, 9, 10, or more amino acid catabolism enzyme genes.

In some embodiments, the genetically engineered bacteria comprise multiple copies of the same amino acid catabolism enzyme gene(s). In some embodiments, the gene encoding the amino acid catabolism enzyme is present on a plasmid and operably linked to a directly or indirectly inducible promoter. In some embodiments, the gene encoding the amino acid catabolism enzyme is present on a plasmid and operably linked to a promoter that is induced under low-oxygen or anaerobic conditions. In some embodiments, the gene encoding the amino acid catabolism enzyme is present on a chromosome and operably linked to a directly or indirectly inducible promoter. In some embodiments, the gene encoding the amino acid catabolism enzyme is present in the chromosome and operably linked to a promoter that is induced under low-oxygen or anaerobic conditions. In some embodiments, the gene encoding the amino acid catabolism enzyme is present on a plasmid and operably linked to a promoter that is induced by exposure to tetracycline or arabinose.

In some embodiments, the promoter that is operably linked to the gene encoding the amino acid catabolism enzyme is directly induced by exogenous environmental conditions. In some embodiments, the promoter that is operably linked to the gene encoding the amino acid catabolism enzyme is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal. In some embodiments, the promoter is directly or indirectly induced by a molecule that is co-administered with the bacterial cell.

In certain embodiments, the bacterial cell comprises a gene encoding an amino acid catabolism enzyme expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters listed in the chart, below. Underlined sequences are predicted ribosome binding sites, and bolded sequences are restriction sites used for cloning.

| FNR Responsive | |
|---|---|
| Promoter | Sequence |
| SEQ ID NO: 1 | GTCAGCATAACACCCTGACCTCTCATTAATTGTTCATGCCGGGCGGCACTATCGTCGTCCGGCCT |
| | TTTCCTCTCTTACTCTGCTACGTACATCTATTTCTATAAATCCGTTCAATTTGTCTGTTTTTTGCACA |
| | AACATGAAATATCAGACAATTCCGTGACTTAAGAAAATTTATACAAATCAGCAATATACCCCTTA |
| | AGGAGTATATAAAGGTGAATTTGATTTACATCAATAAGCGGGGTTGCTGAATCGTTAAGGTAGG |
| | CGGTAATAGAAAAGAAATCGAGGCAAAA |
| SEQ ID NO: 2 | ATTTCCTCTCATCCCATCCGGGGTGAGAGTCTTTTCCCCCGACTTATGGCTCATGCATGCATCAAA |
| | AAAGATGTGAGCTTGATCAAAAACAAAAAATATTTCACTCGACAGGAGTATTTATATTGCGCCCG |
| | TTACGTGGGCTTCGACTGTAAATCAGAAAGGAGAAAACACCT |
| SEQ ID NO: 3 | GTCAGCATAACACCCTGACCTCTCATTAATTGTTCATGCCGGGCGGCACTATCGTCGTCCGGCCT |
| | TTTCCTCTCTTACTCTGCTACGTACATCTATTTCTATAAATCCGTTCAATTTGTCTGTTTTTTGCACA |
| | AACATGAAATATCAGACAATTCCGTGACTTAAGAAAATTTATACAAATCAGCAATATACCCCTTA |
| | AGGAGTATATAAAGGTGAATTTGATTTACATCAATAAGCGGGGTTGCTGAATCGTTAAGGATCC |
| | CTCTAGAAATAATTTTGTTTAACTTTAAGAAGGAGATATACAT |
| SEQ ID NO: 4 | CATTTCCTCTCATCCCATCCGGGGTGAGAGTCTTTTCCCCCGACTTATGGCTCATGCATGCATCAA |
| | AAAAGATGTGAGCTTGATCAAAAACAAAAAATATTTCACTCGACAGGAGTATTTATATTGCGCCC |
| | GGATCCCTCTAGAAATAATTTTGTTTAACTTTAAGAAGGAGATATACAT |
| SEQ ID NO: 5 | AGTTGTTCTTATTGGTGGTGTTGCTTTATGGTTGCATCGTAGTAAATGGTTGTAACAAAAGCAAT |
| | TTTTCCGGCTGTCTGTATACAAAAACGCCGTAAAGTTTGAGCGAAGTCAATAAACTCTCTACCCA |
| | TTCAGGGCAATATCTCTCTTGGATCCCTCTAGAAATAATTTTGTTTAACTTTAAGAAGGAGATATA |
| | CAT |

In one embodiment, the FNR responsive promoter comprises SEQ ID NO:1. In another embodiment, the FNR responsive promoter comprises SEQ ID NO:2. In another embodiment, the FNR responsive promoter comprises SEQ ID NO:3. In another embodiment, the FNR responsive promoter comprises SEQ ID NO:4. In yet another embodiment, the FNR responsive promoter comprises SEQ ID NO:5.

In some embodiments, multiple distinct FNR nucleic acid sequences are inserted in the genetically engineered bacteria. In alternate embodiments, the genetically engineered bacteria comprise a gene encoding an amino acid catabolism enzyme expressed under the control of an alternate oxygen level-dependent promoter, e.g., DNR (Trunk et al., 2010) or ANR (Ray et al., 1997). In these embodiments, expression of the amino acid catabolism enzyme gene is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the amino acid catabolism enzyme, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the amino acid catabolism enzyme, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the amino acid catabolism enzyme, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006).

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the amino acid catabolism enzyme are present on different plasmids. In some embodiments, the gene encoding the oxygen level-sensing transcriptional regulator and the gene encoding the amino acid catabolism enzyme are present on the same plasmid. In some embodiments, the gene encoding the oxygen level-sensing transcriptional regulator is present on a chromosome. In some embodiments, the gene encoding the oxygen level-sensing transcriptional regulator and the gene encoding the amino acid catabolism enzyme are present on different chromosomes. In some embodiments, the gene encoding the oxygen level-sensing transcriptional regulator and the gene encoding the amino acid catabolism enzyme are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the amino acid catabolism enzyme. In some embodiments, expression of the transcriptional regulator is controlled by the same promoter that controls expression of the amino acid catabolism enzyme. In some embodiments, the transcriptional regulator and the amino acid catabolism enzyme are divergently transcribed from a promoter region.

RNS-dependent regulation

In some embodiments, the genetically engineered bacteria or genetically engineered virus comprise a gene encoding an amino acid catabolism enzyme that is expressed under the control of an inducible promoter. In some embodiments, the genetically engineered bacterium or genetically engineered virus that expresses an amino acid catabolism enzyme under the control of a promoter that is activated by inflammatory conditions. In one embodiment, the gene for producing the amino acid catabolism enzyme is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO.), peroxynitrite or peroxynitrite anion (ONOO—), nitrogen dioxide (.NO2), dinitrogen trioxide (N203), peroxynitrous acid (ONOOH), and nitroperoxycarbonate (ONOOCO2-) (unpaired electrons denoted by .). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., an amino acid catabolism enzyme gene sequence(s), e.g., any of the amino acid catabolism enzymes described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or genes, e.g., an amino acid catabolism enzyme gene sequence(s). For example, in the presence of RNS, a transcription factor senses RNS and no longer binds to and/or represses the regulatory region, thereby derepressing an operatively linked gene sequence or gene cassette. Thus, RNS derepresses expression of the gene or genes.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 3.

TABLE 3
Examples of RNS-sensing transcription factors and RNS-responsive
genes

| | Primarily | |
|---|---|---|
| RNS-sensing | capable | Examples of responsive genes, |
| transcription factor: | of sensing: | promoters, and/or regulatory regions: |
| NsrR | NO | norB, aniA, nsrR, hmpA, ytfE, |
| | | ygbA, hcp, hcr, nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of an amino acid catabolism enzyme, thus controlling expression of the amino acid catabolism enzyme relative to RNS levels. For example, the tunable regulatory region is a RNS-inducible regulatory region, and the payload is an amino acid catabolism enzyme, such as any of the amino acid catabolism enzymes provided herein; when RNS is present, e.g., in an inflamed tissue, a RNS-sensing transcription factor binds to and/or activates the regulatory region and drives expression of the amino acid catabolism enzyme gene or genes. Subsequently, when inflammation is ameliorated, RNS levels are reduced, and production of the amino acid catabolism enzyme is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The genetically engineered bacteria of the invention may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012; Table 1). In certain embodiments, the genetically engineered bacteria of the invention comprise a RNS-inducible regulatory region from norVW that is operatively linked to a gene or genes, e.g., one or more amino acid catabolism enzyme gene sequence(s). In the presence of RNS, a NorR transcription factor senses RNS and activates to the norVW regulatory region, thereby driving expression of the operatively linked gene(s) and producing the amino acid catabolism enzyme.

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The genetically engineered bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008; Table 1). In certain embodiments, the genetically engineered bacteria of the invention comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more amino acid catabolism enzymes. In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor that can sense NO and control the expression of genes responsible for NO metabolism” (Isabella et al., 2009). The genetically engineered bacteria of the invention may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010; Table 1). In certain embodiments, the genetically engineered bacteria of the invention comprise a RNS-derepressible regulatory region from norB that is operatively linked to a gene or genes, e.g., an amino acid catabolism enzyme gene or genes. In the presence of RNS, an NsrR transcription factor senses RNS and no longer binds to the norB regulatory region, thereby derepressing the operatively linked an amino acid catabolism enzyme gene or genes and producing the encoding an amino acid catabolism enzyme(s).

In some embodiments, it is advantageous for the genetically engineered bacteria to express a RNS-sensing transcription factor that does not regulate the expression of a significant number of native genes in the bacteria. In some embodiments, the genetically engineered bacterium of the invention expresses a RNS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the genetically engineered bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the genetically engineered bacteria may comprise a two repressor activation regulatory circuit, which is used to express an amino acid catabolism enzyme. The two repressor activation regulatory circuit comprises a first RNS-sensing repressor and a second repressor, which is operatively linked to a gene or gene cassette, e.g., encoding an amino acid catabolism enzyme. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., an amino acid catabolism enzyme gene or genes is expressed.

A RNS-responsive transcription factor may induce, derepress, or repress gene expression depending upon the regulatory region sequence used in the genetically engineered bacteria. One or more types of RNS-sensing transcription factors and corresponding regulatory region sequences may be present in genetically engineered bacteria. In some embodiments, the genetically engineered bacteria comprise one type of RNS-sensing transcription factor, e.g., NsrR, and one corresponding regulatory region sequence, e.g., from norB. In some embodiments, the genetically engineered bacteria comprise one type of RNS-sensing transcription factor, e.g., NsrR, and two or more different corresponding regulatory region sequences, e.g., from norB and aniA. In some embodiments, the genetically engineered bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the genetically engineered bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the genetically engineered bacteria of the invention comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the genetically engineered bacteria of the invention comprise a gene for a RNS-sensing transcription factor from a different species, strain, or substrain of bacteria. In some embodiments, the genetically engineered bacteria comprise a RNS-responsive regulatory region from a different species, strain, or substrain of bacteria. In some embodiments, the genetically engineered bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the genetically engineered bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the genetically engineered bacteria of the invention comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the genetically engineered bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the amino acid catabolism enzyme in the presence of RNS, as compared to the wild-type regulatory region under the same conditions. In some embodiments, the genetically engineered bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the amino acid catabolism enzyme in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the amino acid catabolism enzyme in the presence of RNS.

In some embodiments, the gene or gene cassette for producing the anti-inflammation and/or gut barrier function enhancer molecule is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of one or more encoding an amino acid catabolism enzyme gene(s) may be integrated into the bacterial chromosome. Having multiple copies of the gene or gen(s) integrated into the chromosome allows for greater production of the amino acid catabolism enzyme(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

ROS-Dependent Regulation

In some embodiments, the genetically engineered bacteria or genetically engineered virus comprise a gene for producing an amino acid catabolism enzyme that is expressed under the control of an inducible promoter. In some embodiments, the genetically engineered bacterium or genetically engineered virus that expresses an amino acid catabolism enzyme under the control of a promoter that is activated by conditions of cellular damage. In one embodiment, the gene for producing the amino acid catabolism enzyme is expressed under the control of an cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H₂O₂), organic peroxide (ROOH), hydroxyl ion (OH—), hydroxyl radical (.OH), superoxide or superoxide anion (.O2-), singlet oxygen (1O2), ozone (O3), carbonate radical, peroxide or peroxyl radical (.O2-2), hypochlorous acid (HOCl), hypochlorite ion (OCl—), sodium hypochlorite (NaOCl), nitric oxide (NO.), and peroxynitrite or peroxynitrite anion (ONOO—) (unpaired electrons denoted by .). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more amino acid catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or genes, e.g., one or more genes encoding one or more amino acid catabolism enzyme(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 4.

TABLE 4
Examples of ROS-sensing transcription factors and ROS-responsive
genes

| ROS-sensing | Primarily | |
|---|---|---|
| transcription | capable of | Examples of responsive genes, |
| factor: | sensing: | promoters, and/or regulatory regions: |
| OxyR | H₂O₂ | ahpC; ahpF; dps; dsbG; JhuF; flu; fur; |
| | | gor; grxA; hemH; katG; oxyS; sufA; |
| | | sufB; sufC; sufD; sufE; sufS; trxC; uxuA; |
| | | yaaA; yaeH; yaiA; ybjM; ydcH; ydeN; |
| | | ygaQ; yljA; ytfK |
| PerR | H₂O₂ | katA; ahpCF; mrgA; zoaA; fur; |
| | | hemAXCDBL; srfA |
| OhrR | Organic | ohrA |
| | peroxides | |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; |
| | | tnp2d; phoD; tnp15b; pstA; tnp5b; xylC; |
| | | gabD1; rluC2; cgtS9; azlC; narKGHJI; |
| | | rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of an amino acid catabolism enzyme, thus controlling expression of the amino acid catabolism enzyme relative to ROS levels. For example, the tunable regulatory region is a ROS-inducible regulatory region, and the molecule is an amino acid catabolism enzyme; when ROS is present, e.g., in an inflamed tissue, a ROS-sensing transcription factor binds to and/or activates the regulatory region and drives expression of the gene sequence for the amino acid catabolism enzyme, thereby producing the amino acid catabolism enzyme. Subsequently, when inflammation is ameliorated, ROS levels are reduced, and production of the amino acid catabolism enzyme is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H2O2 detoxification (katE, ahpCF), heme biosynthesis (hemH), reductant supply (grxA, gor, trxC), thiol-disulfide isomerization (dsbG), Fe-S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012; Table 1). In certain embodiments, the genetically engineered bacteria of the invention comprise a ROS-inducible regulatory region from oxyS that is operatively linked to a gene, e.g., an amino acid catabolism enzyme gene. In the presence of ROS, e.g., H2O2, an OxyR transcription factor senses ROS and activates to the oxyS regulatory region, thereby driving expression of the operatively linked amino acid catabolism enzyme gene and producing the amino acid catabolism enzyme. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The genetically engineered bacteria of the invention may comprise any suitable ROS-responsive regulatory region from a gene that is activated by SoxR. Genes that are capable of being activated by SoxR are known in the art (see, e.g., Koo et al., 2003; Table 1). In certain embodiments, the genetically engineered bacteria of the invention comprise a ROS-inducible regulatory region from soxS that is operatively linked to a gene, e.g., an amino acid catabolism enzyme. In the presence of ROS, the SoxR transcription factor senses ROS and activates the soxS regulatory region, thereby driving expression of the operatively linked an amino acid catabolism enzyme gene and producing the an amino acid catabolism enzyme.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H2O2 but it shows much higher reactivity for organic hydroperoxides” (Duarte et al., 2010). The genetically engineered bacteria of the invention may comprise any suitable ROS-responsive regulatory region from a gene that is repressed by OhrR. Genes that are capable of being repressed by OhrR are known in the art (see, e.g., Dubbs et al., 2012; Table 1). In certain embodiments, the genetically engineered bacteria of the invention comprise a ROS-derepressible regulatory region from ohrA that is operatively linked to a gene or gene cassette, e.g., an amino acid catabolism enzyme gene. In the presence of ROS, e.g., NaOCl, an OhrR transcription factor senses ROS and no longer binds to the ohrA regulatory region, thereby derepressing the operatively linked amino acid catabolism enzyme gene and producing the an amino acid catabolism enzyme.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the genetically engineered bacteria of the invention comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA” and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The genetically engineered bacteria of the invention may comprise any suitable ROS-responsive regulatory region from a gene that is repressed by RosR. Genes that are capable of being repressed by RosR are known in the art (see, e.g., Bussmann et al., 2010; Table 1). In certain embodiments, the genetically engineered bacteria of the invention comprise a ROS-derepressible regulatory region from cgtS9 that is operatively linked to a gene or gene cassette, e.g., an amino acid catabolism enzyme. In the presence of ROS, e.g., H2O2, a RosR transcription factor senses ROS and no longer binds to the cgtS9 regulatory region, thereby derepressing the operatively linked amino acid catabolism enzyme gene and producing the amino acid catabolism enzyme.

In some embodiments, it is advantageous for the genetically engineered bacteria to express a ROS-sensing transcription factor that does not regulate the expression of a significant number of native genes in the bacteria. In some embodiments, the genetically engineered bacterium of the invention expresses a ROS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the genetically engineered bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA) residing within and near the promoter sequences of PerR-controlled genes” (Marinho et al., 2014). PerR is capable of binding a regulatory region that “overlaps part of the promoter or is immediately downstream from it” (Dubbs et al., 2012). The genetically engineered bacteria of the invention may comprise any suitable ROS-responsive regulatory region from a gene that is repressed by PerR. Genes that are capable of being repressed by PerR are known in the art (see, e.g., Dubbs et al., 2012; Table 1).

In these embodiments, the genetically engineered bacteria may comprise a two repressor activation regulatory circuit, which is used to express an amino acid catabolism enzyme. The two repressor activation regulatory circuit comprises a first ROS-sensing repressor, e.g., PerR, and a second repressor, e.g., TetR, which is operatively linked to a gene or gene cassette, e.g., an amino acid catabolism enzyme. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., an amino acid catabolism enzyme. In the absence of PerR binding (which occurs in the absence of ROS), tetR is transcribed, and TetR represses expression of the gene or gene cassette, e.g., an amino acid catabolism enzyme. In the presence of PerR binding (which occurs in the presence of ROS), tetR expression is repressed, and the gene or gene cassette, e.g., an amino acid catabolism enzyme, is expressed.

A ROS-responsive transcription factor may induce, derepress, or repress gene expression depending upon the regulatory region sequence used in the genetically engineered bacteria. For example, although “OxyR is primarily thought of as a transcriptional activator under oxidizing conditions . . . OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The genetically engineered bacteria of the invention may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 1). Or, for example, although RosR is capable of repressing a number of genes, it is also capable of activating certain genes, e.g., the narKGHJI operon. In some embodiments, the genetically engineered bacteria comprise any suitable ROS-responsive regulatory region from a gene that is activated by RosR. In addition, “PerR-mediated positive regulation has also been observed . . . and appears to involve PerR binding to distant upstream sites” (Dubbs et al., 2012). In some embodiments, the genetically engineered bacteria comprise any suitable ROS-responsive regulatory region from a gene that is activated by PerR.

One or more types of ROS-sensing transcription factors and corresponding regulatory region sequences may be present in genetically engineered bacteria. For example, “OhrR is found in both Gram-positive and Gram-negative bacteria and can coreside with either OxyR or PerR or both” (Dubbs et al., 2012). In some embodiments, the genetically engineered bacteria comprise one type of ROS-sensing transcription factor, e.g., OxyR, and one corresponding regulatory region sequence, e.g., from oxyS. In some embodiments, the genetically engineered bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the genetically engineered bacteria comprise two or more types of ROS-sensing transcription factors and one corresponding regulatory region sequence.

Nucleic acid sequences of several exemplary OxyR-regulated regulatory regions are shown in Table 5. OxyR binding sites are underlined and bolded. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 46, 47, 48, or 49, or a functional fragment thereof.

TABLE 5
Nucleotide sequences of exemplary OxyR-regulated
regulatory regions

| Regulatory | |
|---|---|
| sequence | 01234567890123456789012345678901234567890123456789 |
| katG | TGTGGCTTTTATGAAAATCACACAGTGATCACAAATTTTAAACA |
| (SEQ ID NO: | GAGCACAAAATGCTGCCTCGAAATGAGGGCGGGAAAATAAGGT |
| 46) | TATCAGCCTTGTTTTCTCCCTCATTACTTGAAGGATATGAAGCTA |
| | AAACCCTTTTTTATAAAGCATTTGTCCGAATTCGGACATAATCA |
| | AAAAAGCTTAATTAAGATCAATTTGATCTACATCTCTTTAACCA |
| | ACAATATGTAAGATCTCAACTATCGCATCCGTGGATTAATTC |
| | AATTATAACTTCTCTCTAACGCTGTGTATCGTAACGGTAACACT |
| | GTAGAGGGGAGCACATTGATGCGAATTCATTAAAGAGGAGAAA |
| | GGTACC |
| dps | TTCCGAAAATTCCTGGCGAGCAGATAAATAAGAATTGTTCTTAT |
| (SEQ ID NO: | CAATATATCTAACTCATTGAATCTTTATTAGTTTTGTTTTTCACG |
| 47) | CTTGTTACCACTATTAGTGTGATAGGAACAGCCAGAATAGCG |
| | GAACACATAGCCGGTGCTATACTTAATCTCGTTAATTACTGGGA |
| | CATAACATCAAGAGGATATGAAATTCGAATTCATTAAAGAGGA |
| | GAAAGGTACC |
| ahpC | GCTTAGATCAGGTGATTGCCCTTTGTTTATGAGGGTGTTGTAATC |
| (SEQ ID NO: | CATGTCGTTGTTGCATTTGTAAGGGCAACACCTCAGCCTGCAGG |
| 48) | CAGGCACTGAAGATACCAAAGGGTAGTTCAGATTACACGGTCA |
| | CCTGGAAAGGGGGCCATTTTACTTTTTATCGCCGCTGGCGGTGC |
| | AAAGTTCACAAAGTTGTCTTACGAAGGTTGTAAGGTAAAACTT |
| | ATCGATTTGATAATGGAAACGCATTAGCCGAATCGGCAAAAAT |
| | TGGTTACCTTACATCTCATCGAAAACACGGAGGAAGTATAGATG |
| | CGAATTCATTAAAGAGGAGAAAGGTACC |
| oxyS | CTCGAGTTCATTATCCATCCTCCATCGCCACGATAGTTCATGGC |
| (SEQ ID NO: | GATAGGTAGAATAGCAATGAACGATTATCCCTATCAAGCATTC |
| 49) | TGACTGATAATTGCTCACACGAATTCATTAAAGAGGAGAAAGGT |
| | ACC |

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the genetically engineered bacteria of the invention comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the genetically engineered bacteria of the invention comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the amino acid catabolism enzyme in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the amino acid catabolism enzyme in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the amino acid catabolism enzyme in the presence of ROS.

In some embodiments, the gene or gene cassette for producing the amino acid catabolism enzyme is present on a plasmid and operably linked to a promoter that is induced by ROS. In some embodiments, the gene or gene cassette for producing the amino acid catabolism enzyme is present in the chromosome and operably linked to a promoter that is induced by ROS. In some embodiments, the gene or gene cassette for producing the amino acid catabolism enzyme is present on a chromosome and operably linked to a promoter that is induced by exposure to tetracycline. In some embodiments, the gene or gene cassette for producing the amino acid catabolism enzyme is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing an amino acid catabolism enzyme(s). In some embodiments, the gene(s) capable of producing an amino acid catabolism enzyme(s) is present on a plasmid and operatively linked to a ROS-responsive regulatory region. In some embodiments, the gene(s) capable of producing an amino acid catabolism enzyme is present in a chromosome and operatively linked to a ROS-responsive regulatory region.

Thus, in some embodiments, the genetically engineered bacteria or genetically engineered virus produce one or more amino acid catabolism enzymes under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing an amino acid catabolism enzyme, such that the amino acid catabolism enzyme can be expressed in the host cell, and the host cell is capable of survival and/or growth in vitro, e.g., in medium, and/or in vivo. In some embodiments, a bacterium may comprise multiple copies of the gene encoding the amino acid catabolism enzyme. In some embodiments, the gene encoding the amino acid catabolism enzyme is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the amino acid catabolism enzyme is expressed on a high-copy plasmid. In some embodiments, the high-copy plasmid may be useful for increasing expression of the amino acid catabolism enzyme. In some embodiments, the gene encoding the amino acid catabolism enzyme is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular amino acid catabolism enzyme inserted at four different insertion sites. Alternatively, the genetically engineered bacteria may include three copies of the gene encoding a particular amino acid catabolism enzyme inserted at three different insertion sites and three copies of the gene encoding a different amino acid catabolism enzyme inserted at three different insertion sites.

In some embodiments, under conditions where the amino acid catabolism enzyme is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the amino acid catabolism enzyme, and/or transcript of the gene(s) in the operon as compared to unmodified bacteria of the same subtype under the same conditions.

In some embodiments, quantitative PCR (qPCR) is used to amplify, detect, and/or quantify mRNA expression levels of the amino acid catabolism enzyme gene(s). Primers specific for amino acid catabolism enzyme the gene(s) may be designed and used to detect mRNA in a sample according to methods known in the art. In some embodiments, a fluorophore is added to a sample reaction mixture that may contain amino acid catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the amino acid catabolism enzyme gene(s).

In some embodiments, quantitative PCR (qPCR) is used to amplify, detect, and/or quantify mRNA expression levels of the amino acid catabolism enzyme gene(s). Primers specific for amino acid catabolism enzyme the gene(s) may be designed and used to detect mRNA in a sample according to methods known in the art. In some embodiments, a fluorophore is added to a sample reaction mixture that may contain amino acid catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the amino acid catabolism enzyme gene(s).

In other embodiments, the inducible promoter is a propionate responsive promoter. For example, the prpR promoter is a propionate responsive promoter. In one embodiment, the propionate responsive promoter comprises SEQ ID NO:106.

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37: D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

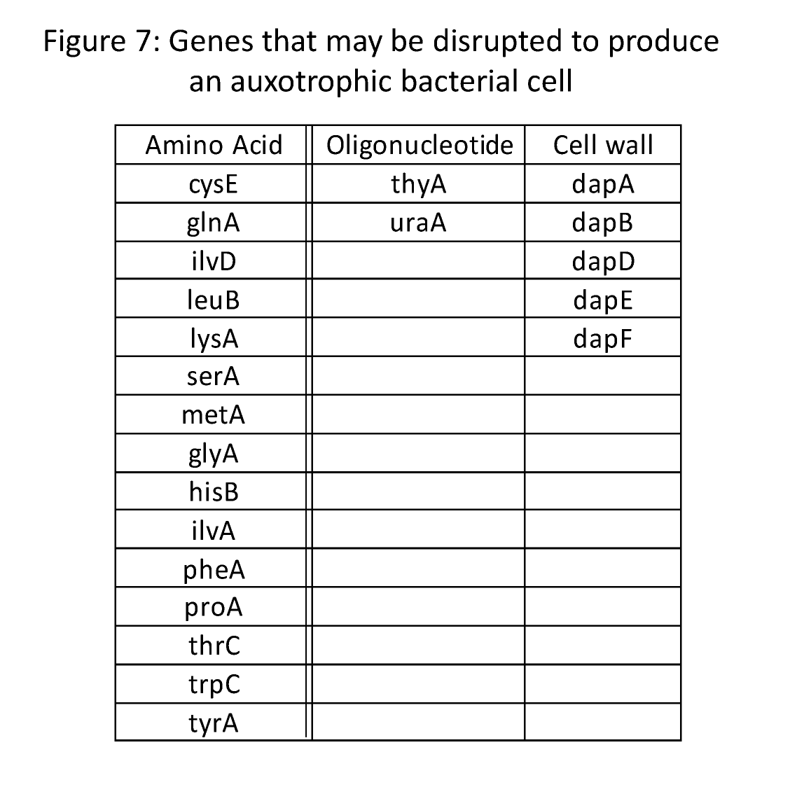

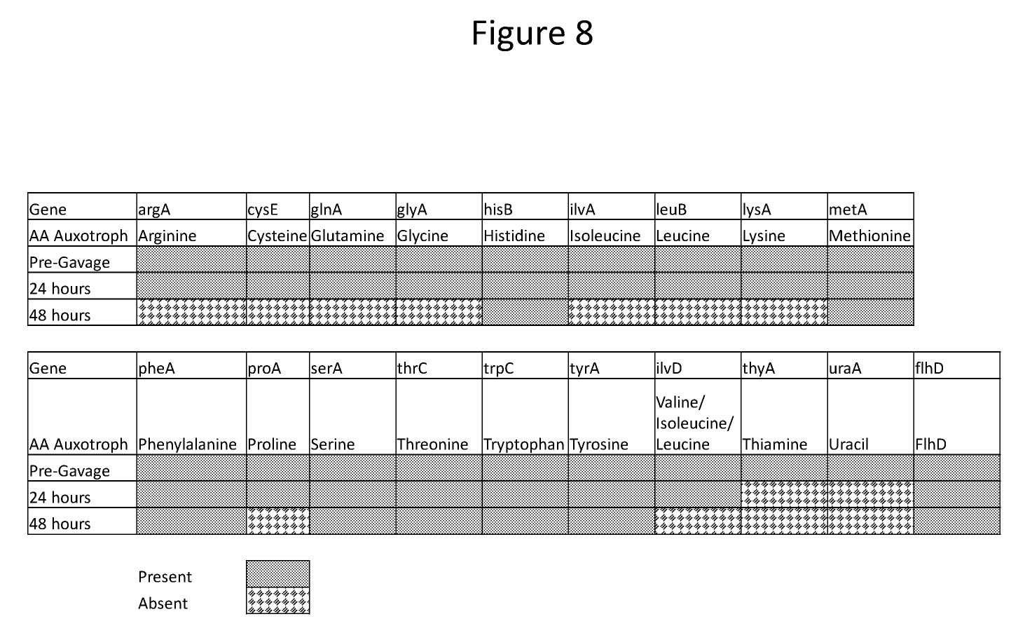

An auxotrophic modification is intended to cause bacteria to die in the absence of an exogenously added nutrient essential for survival or growth because they lack the gene(s) necessary to produce that essential nutrient (see FIGS. 7 and 8). In some embodiments, any of the genetically engineered bacteria described herein also comprise a deletion or mutation in a gene required for cell survival and/or growth. In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria. For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. Δ thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, mc, ftsB, eno, pyrG, chpR, lgt, fbaA, pgk, yqgD, metK, yqgF, plsC, ygiT, pare, ribB, cca, ygjD, tdcF, yraL, yihA, ftsN, murl, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, lspA, ispH, dapB, folA, imp, yabQ, ftsL, ftsl, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsl, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsK, rplE, rplX, rplN, rpsQ, rpmC, rplP, rpsL, rplV, rpsS, rplB, cdsA, yaeL, yaeT, lpxD, fabZ, lpxA, lpxB, dnaE, accA, tilS, proS, yafF, tsf, pyrH, olA, rlpB, leuS, int, glnS, fldA, cydA, infA, cydC, ftsK, lolA, serS, rpsA, msbA, lpxK, kdsB, mukF, mukE, mukB, asnS, fabA, mviN, me, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabl, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4: 307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

Genetic Regulatory Circuits

In some embodiments, the genetically engineered bacteria comprise multi-layered genetic regulatory circuits for expressing the constructs described herein (see, e.g., U.S. Provisional Application No. 62/184,811, incorporated herein by reference in its entirety). The genetic regulatory circuits are useful to screen for mutant bacteria that produce an amino acid catabolism enzyme or rescue an auxotroph. In certain embodiments, the invention provides methods for selecting genetically engineered bacteria that produce one or more genes of interest.

In some embodiments, the invention provides genetically engineered bacteria comprising a gene or gene cassette for producing a payload and a T7 polymerase-regulated genetic regulatory circuit. For example, the genetically engineered bacteria comprise a first gene encoding a T7 polymerase, wherein the first gene is operably linked to a fumarate and nitrate reductase regulator (FNR)-responsive promoter; a second gene or gene cassette for producing a payload, wherein the second gene or gene cassette is operably linked to a T7 promoter that is induced by the T7 polymerase; and a third gene encoding an inhibitory factor, lysY, that is capable of inhibiting the T7 polymerase. In the presence of oxygen, FNR does not bind the FNR-responsive promoter, and the payload is not expressed. LysY is expressed constitutively (P-lac constitutive) and further inhibits T7 polymerase. In the absence of oxygen, FNR dimerizes and binds to the FNR-responsive promoter, T7 polymerase is expressed at a level sufficient to overcome lysY inhibition, and the payload is expressed. In some embodiments, the lysY gene is operably linked to an additional FNR binding site. In the absence of oxygen, FNR dimerizes to activate T7 polymerase expression as described above, and also inhibits lysY expression.

In some embodiments, the invention provides genetically engineered bacteria comprising a gene or gene cassette for producing a payload and a protease-regulated genetic regulatory circuit. For example, the genetically engineered bacteria comprise a first gene encoding an mf-lon protease, wherein the first gene is operably linked to a FNR-responsive promoter; a second gene or gene cassette for producing a payload operably linked to a tet regulatory region (tetO); and a third gene encoding an mf-lon degradation signal linked to a tet repressor (tetR), wherein the tetR is capable of binding to the tet regulatory region and repressing expression of the second gene or gene cassette. The mf-lon protease is capable of recognizing the mf-lon degradation signal and degrading the tetR. In the presence of oxygen, FNR does not bind the FNR-responsive promoter, the repressor is not degraded, and the payload is not expressed. In the absence of oxygen, FNR dimerizes and binds the FNR-responsive promoter, thereby inducing expression of mf-lon protease. The mf-lon protease recognizes the mf-lon degradation signal and degrades the tetR, and the payload is expressed.

In some embodiments, the invention provides genetically engineered bacteria comprising a gene or gene cassette for producing a payload and a repressor-regulated genetic regulatory circuit. For example, the genetically engineered bacteria comprise a first gene encoding a first repressor, wherein the first gene is operably linked to a FNR-responsive promoter; a second gene or gene cassette for producing a payload operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the payload is not expressed. In the absence of oxygen, FNR dimerizes and binds the FNR-responsive promoter, the first repressor is expressed, the second repressor is not expressed, and the payload is expressed.

Examples of repressors useful in these embodiments include, but are not limited to, ArgR, TetR, ArsR, AscG, LacI, CscR, DeoR, DgoR, FruR, GalR, GatR, CI, LexA, RafR, QacR, and PtxS (US20030166191).

In some embodiments, the invention provides genetically engineered bacteria comprising a gene or gene cassette for producing a payload and a regulatory RNA-regulated genetic regulatory circuit. For example, the genetically engineered bacteria comprise a first gene encoding a regulatory RNA, wherein the first gene is operably linked to a FNR-responsive promoter, and a second gene or gene cassette for producing a payload. The second gene or gene cassette is operably linked to a constitutive promoter and further linked to a nucleotide sequence capable of producing an mRNA hairpin that inhibits translation of the payload. The regulatory RNA is capable of eliminating the mRNA hairpin and inducing payload translation via the ribosomal binding site. In the presence of oxygen, FNR does not bind the FNR-responsive promoter, the regulatory RNA is not expressed, and the mRNA hairpin prevents the payload from being translated. In the absence of oxygen, FNR dimerizes and binds the FNR-responsive promoter, the regulatory RNA is expressed, the mRNA hairpin is eliminated, and the payload is expressed.

In some embodiments, the invention provides genetically engineered bacteria comprising a gene or gene cassette for producing a payload and a CRISPR-regulated genetic regulatory circuit. For example, the genetically engineered bacteria comprise a Cas9 protein; a first gene encoding a CRISPR guide RNA, wherein the first gene is operably linked to a FNR-responsive promoter; a second gene or gene cassette for producing a payload, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the payload is not expressed. In the absence of oxygen, FNR dimerizes and binds the FNR-responsive promoter, the guide RNA is expressed, the repressor is not expressed, and the payload is expressed.

In some embodiments, the invention provides genetically engineered bacteria comprising a gene or gene cassette for producing a payload and a recombinase-regulated genetic regulatory circuit. For example, the genetically engineered bacteria comprise a first gene encoding a recombinase, wherein the first gene is operably linked to a FNR-responsive promoter, and a second gene or gene cassette for producing a payload operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the payload remains in the 3′ to 5′ orientation, and no functional payload is produced. In the absence of oxygen, FNR dimerizes and binds the FNR-responsive promoter, the recombinase is expressed, the payload is reverted to the 5′ to 3′ orientation, and functional payload is produced.

In some embodiments, the invention provides genetically engineered bacteria comprising a gene or gene cassette for producing a payload and a polymerase- and recombinase-regulated genetic regulatory circuit. For example, the genetically engineered bacteria comprise a first gene encoding a recombinase, wherein the first gene is operably linked to a FNR-responsive promoter; a second gene or gene cassette for producing a payload operably linked to a T7 promoter; a third gene encoding a T7 polymerase, wherein the T7 polymerase is capable of binding to the T7 promoter and inducing expression of the payload. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the payload is not expressed. In the absence of oxygen, FNR dimerizes and binds the FNR-responsive promoter, the recombinase is expressed, the T7 polymerase gene is reverted to the 5′ to 3′ orientation, and the payload is expressed.

Kill Switches

In some embodiments, the genetically engineered bacteria also comprise a kill switch (see, e.g., U.S. Provisional Application Nos. 62/183,935 and 62/263,329, each of which are expressly incorporated herein by reference in their entireties). The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

Bacteria engineered with kill switches have been engineered for in vitro research purposes, e.g., to limit the spread of a biofuel-producing microorganism outside of a laboratory environment. Bacteria engineered for in vivo administration to treat a disease or disorder may also be programmed to die at a specific time after the expression and delivery of a heterologous gene or genes, for example, a therapeutic gene(s) or after the subject has experienced the therapeutic effect. For example, in some embodiments, the kill switch is activated to kill the bacteria after a period of time following expression of an amino acid catabolism enzyme. In some embodiments, the kill switch is activated in a delayed fashion following expression of the amino acid catabolism gene, for example, after the production of the amino acid catabolism enzyme. Alternatively, the bacteria may be engineered to die after the bacteria has spread outside of a disease site. Specifically, it may be useful to prevent long-term colonization of subjects by the microorganism, spread of the microorganism outside the area of interest (for example, outside the gut) within the subject, or spread of the microorganism outside of the subject into the environment (for example, spread to the environment through the stool of the subject).

Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al., 2000), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al., 2010). In some embodiments, the kill switch is activated to kill the bacteria after a period of time following oxygen level-dependent expression of an amino acid catabolism enzyme. In some embodiments, the kill switch is activated in a delayed fashion following oxygen level-dependent expression of an amino acid catabolism enzyme.

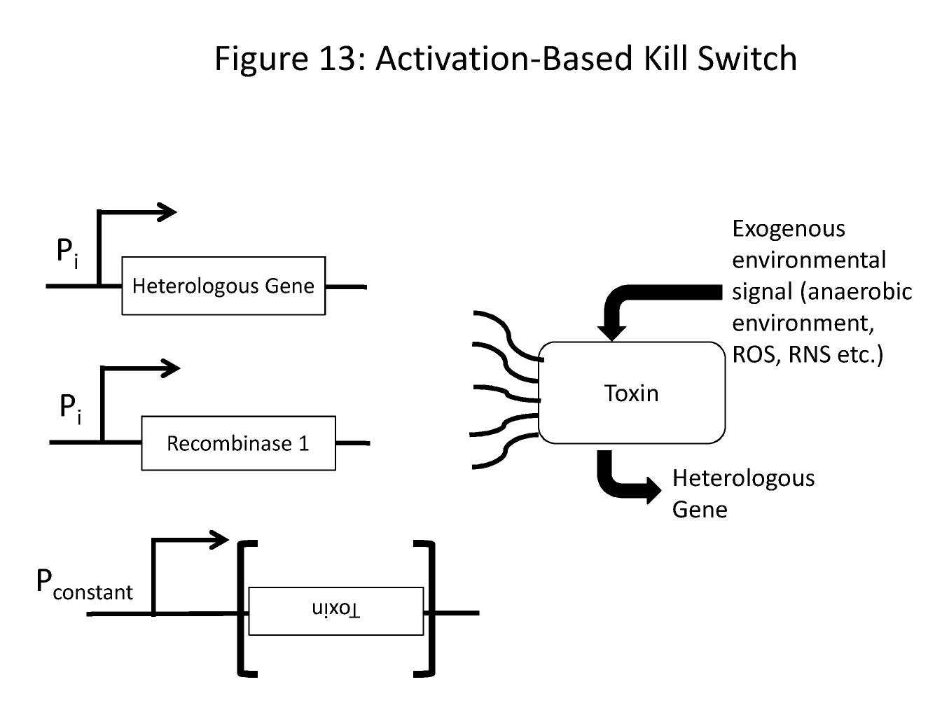

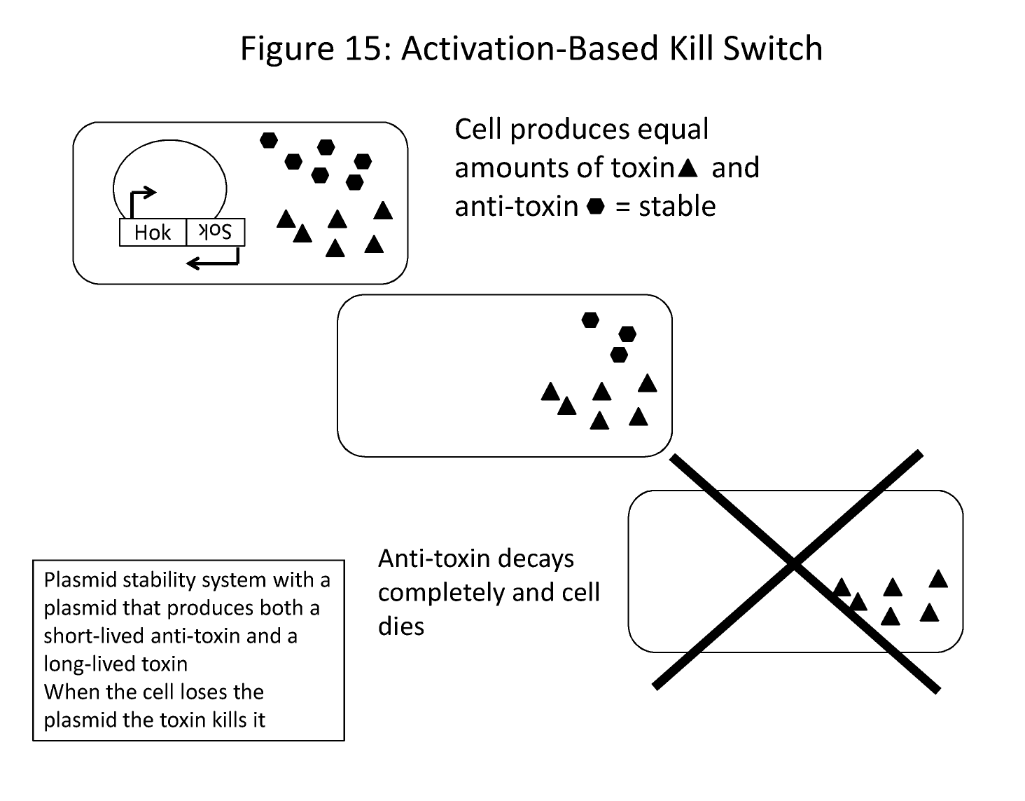

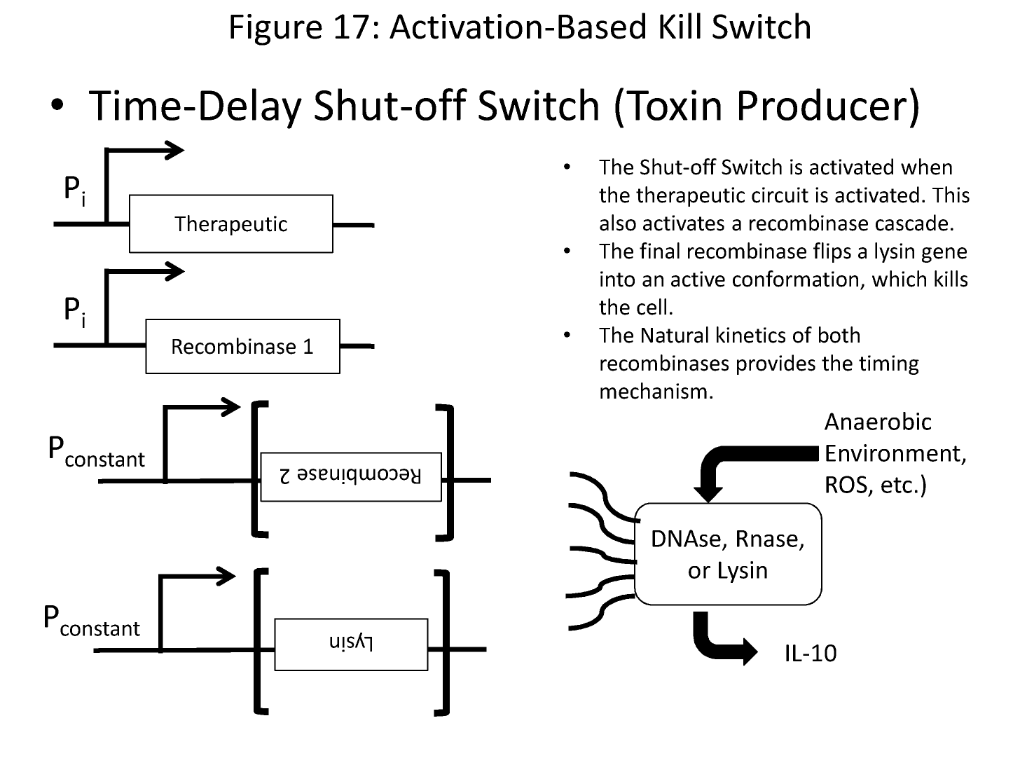

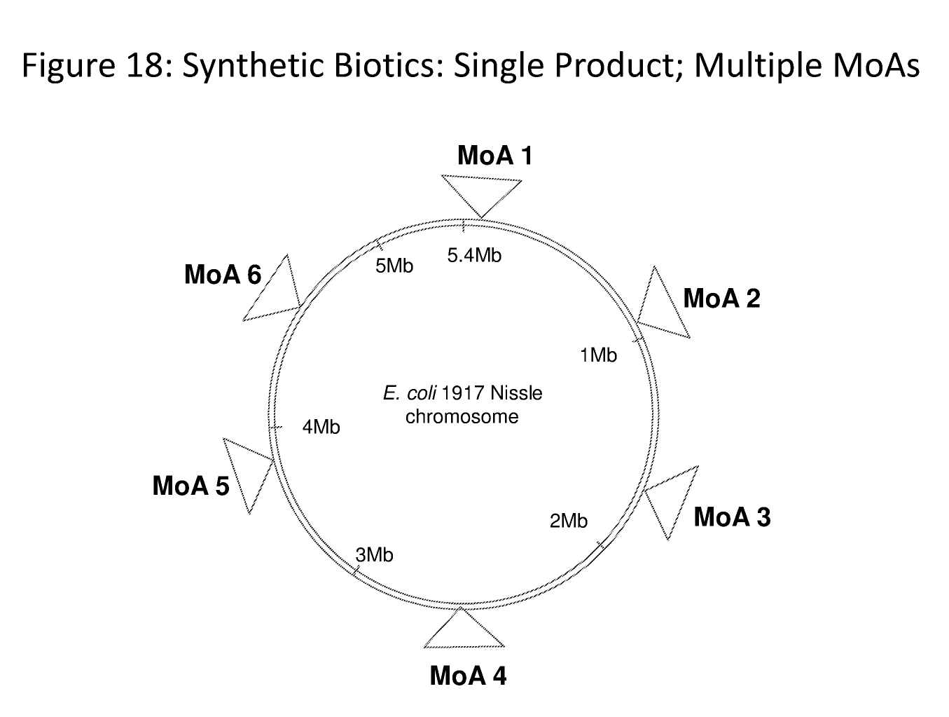

Kill-switches can be designed such that a toxin is produced in response to an environmental condition or external signal (e.g., the bacteria is killed in response to an external cue; i.e., an activation-based kill switch, see FIG. 13-17) or, alternatively designed such that a toxin is produced once an environmental condition no longer exists or an external signal is ceased (i.e., a repression-based kill switch, see FIGS. 9-12).

Thus, in some embodiments, the genetically engineered bacteria of the disclosure are further programmed to die after sensing an exogenous environmental signal, for example, in a low oxygen environment. In some embodiments, the genetically engineered bacteria of the present disclosure, e.g., bacteria expressing an amino acid catabolism enzyme, comprise one or more genes encoding one or more recombinase(s), whose expression is induced in response to an environmental condition or signal and causes one or more recombination events that ultimately leads to the expression of a toxin which kills the cell. In some embodiments, the at least one recombination event is the flipping of an inverted heterologous gene encoding a bacterial toxin which is then constitutively expressed after it is flipped by the first recombinase. In one embodiment, constitutive expression of the bacterial toxin kills the genetically engineered bacterium. In these types of kill-switch systems once the engineered bacterial cell senses the exogenous environmental condition and expresses the heterologous gene of interest, the recombinant bacterial cell is no longer viable.

In another embodiment in which the genetically engineered bacteria of the present disclosure, e.g., bacteria expressing an amino acid catabolism enzyme, express one or more recombinase(s) in response to an environmental condition or signal causing at least one recombination event, the genetically engineered bacterium further expresses a heterologous gene encoding an anti-toxin in response to an exogenous environmental condition or signal. In one embodiment, the at least one recombination event is flipping of an inverted heterologous gene encoding a bacterial toxin by a first recombinase. In one embodiment, the inverted heterologous gene encoding the bacterial toxin is located between a first forward recombinase recognition sequence and a first reverse recombinase recognition sequence. In one embodiment, the heterologous gene encoding the bacterial toxin is constitutively expressed after it is flipped by the first recombinase. In one embodiment, the anti-toxin inhibits the activity of the toxin, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In another embodiment, the at least one recombination event is flipping of an inverted heterologous gene encoding a second recombinase by a first recombinase, followed by the flipping of an inverted heterologous gene encoding a bacterial toxin by the second recombinase. In one embodiment, the inverted heterologous gene encoding the second recombinase is located between a first forward recombinase recognition sequence and a first reverse recombinase recognition sequence. In one embodiment, the inverted heterologous gene encoding the bacterial toxin is located between a second forward recombinase recognition sequence and a second reverse recombinase recognition sequence. In one embodiment, the heterologous gene encoding the second recombinase is constitutively expressed after it is flipped by the first recombinase. In one embodiment, the heterologous gene encoding the bacterial toxin is constitutively expressed after it is flipped by the second recombinase. In one embodiment, the genetically engineered bacterium is killed by the bacterial toxin. In one embodiment, the genetically engineered bacterium further expresses a heterologous gene encoding an anti-toxin in response to the exogenous environmental condition. In one embodiment, the anti-toxin inhibits the activity of the toxin when the exogenous environmental condition is present, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In one embodiment, the at least one recombination event is flipping of an inverted heterologous gene encoding a second recombinase by a first recombinase, followed by flipping of an inverted heterologous gene encoding a third recombinase by the second recombinase, followed by flipping of an inverted heterologous gene encoding a bacterial toxin by the third recombinase. Accordingly, in one embodiment, the disclosure provides at least 1, 2, 3, 4, 5, 6, 7, 8, 9, 10, 11, 12, 13, 14, 15, 16, 17, 18, 19, or 20 recombinases that can be used serially.

In one embodiment, the at least one recombination event is flipping of an inverted heterologous gene encoding a first excision enzyme by a first recombinase. In one embodiment, the inverted heterologous gene encoding the first excision enzyme is located between a first forward recombinase recognition sequence and a first reverse recombinase recognition sequence. In one embodiment, the heterologous gene encoding the first excision enzyme is constitutively expressed after it is flipped by the first recombinase. In one embodiment, the first excision enzyme excises a first essential gene. In one embodiment, the programmed recombinant bacterial cell is not viable after the first essential gene is excised.

In one embodiment, the first recombinase further flips an inverted heterologous gene encoding a second excision enzyme. In one embodiment, the wherein the inverted heterologous gene encoding the second excision enzyme is located between a second forward recombinase recognition sequence and a second reverse recombinase recognition sequence. In one embodiment, the heterologous gene encoding the second excision enzyme is constitutively expressed after it is flipped by the first recombinase. In one embodiment, the genetically engineered bacterium dies or is no longer viable when the first essential gene and the second essential gene are both excised. In one embodiment, the genetically engineered bacterium dies or is no longer viable when either the first essential gene is excised or the second essential gene is excised by the first recombinase.

In one embodiment, the first excision enzyme is Xis1. In one embodiment, the first excision enzyme is Xis2. In one embodiment, the first excision enzyme is Xis1, and the second excision enzyme is Xis2.

In one embodiment, the genetically engineered bacterium dies after the at least one recombination event occurs. In another embodiment, the genetically engineered bacterium is no longer viable after the at least one recombination event occurs.

In any of these embodiment, the recombinase can be a recombinase selected from the group consisting of: BxbI, PhiC31, TP901, BxbI, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34, or a biologically active fragment thereof.

In the above-described kill-switch circuits, a toxin is produced in the presence of an environmental factor or signal. In another aspect of kill-switch circuitry, a toxin may be repressed in the presence of an environmental factor (not produced) and then produced once the environmental condition or external signal is no longer present. Such kill switches are called repression-based kill switches and represent systems in which the bacterial cells are viable only in the presence of an external factor or signal, such as arabinose or other sugar. Exemplary kill switch designs in which the toxin is repressed in the presence of an external factor or signal (and activated once the external signal is removed) is shown in FIGS. 9-12. The disclosure provides recombinant bacterial cells which express one or more heterologous gene(s) upon sensing arabinose or other sugar in the exogenous environment. In this aspect, the recombinant bacterial cells contain the araC gene, which encodes the AraC transcription factor, as well as one or more genes under the control of the araBAD promoter. In the absence of arabinose, the AraC transcription factor adopts a conformation that represses transcription of genes under the control of the araBAD promoter. In the presence of arabinose, the AraC transcription factor undergoes a conformational change that allows it to bind to and activate the araBAD promoter, which induces expression of the desired gene, for example tetR, which represses expression of a toxin gene. In this embodiment, the toxing gene is repressed in the presence of arabinose or other sugar. In an environment where arabinose is not present, the tetR gene is not activated and the toxin is expressed, thereby killing the bacteria. The arabinose system can also be used to express an essential gene, in which the essential gene is only expressed in the presence of arabinose or other sugar and is not expressed when arabinose or other sugar is absent from the environment.

Thus, in some embodiments in which one or more heterologous gene(s) are expressed upon sensing arabinose in the exogenous environment, the one or more heterologous genes are directly or indirectly under the control of the araBAD promoter. In some embodiments, the expressed heterologous gene is selected from one or more of the following: a heterologous therapeutic gene, a heterologous gene encoding an antitoxin, a heterologous gene encoding a repressor protein or polypeptide, for example, a TetR repressor, a heterologous gene encoding an essential protein not found in the bacterial cell, and/or a heterologous encoding a regulatory protein or polypeptide.

Arabinose inducible promoters are known in the art, including Pₐᵣₐ, P_{araB}, P_{araC}, and P_{araBAD}. In one embodiment, the arabinose inducible promoter is from E. coli. In some embodiments, the P_{araC} promoter and the P_{araBAD} promoter operate as a bidirectional promoter, with the P_{araBAD} promoter controlling expression of a heterologous gene(s) in one direction, and the P_{araC} (in close proximity to, and on the opposite strand from the P_{araBAD} promoter), controlling expression of a heterologous gene(s) in the other direction. In the presence of arabinose, transcription of both heterologous genes from both promoters is induced. However, in the absence of arabinose, transcription of both heterologous genes from both promoters is not induced.

In one exemplary embodiment of the disclosure, the engineered bacteria of the present disclosure contains a kill-switch having at least the following sequences: a P_{araBAD} promoter operably linked to a heterologous gene encoding a Tetracycline Repressor Protein (TetR), a P_{araC} promoter operably linked to a heterologous gene encoding AraC transcription factor, and a heterologous gene encoding a bacterial toxin operably linked to a promoter which is repressed by the Tetracycline Repressor Protein (P_{TetR}). In the presence of arabinose, the AraC transcription factor activates the P_{araBAD} promoter, which activates transcription of the TetR protein which, in turn, represses transcription of the toxin. In the absence of arabinose, however, AraC suppresses transcription from the P_{araBAD} promoter and no TetR protein is expressed. In this case, expression of the heterologous toxin gene is activated, and the toxin is expressed. The toxin builds up in the recombinant bacterial cell, and the recombinant bacterial cell is killed. In one embodiment, the araC gene encoding the AraC transcription factor is under the control of a constitutive promoter and is therefore constitutively expressed.

In one embodiment of the disclosure, the recombinant bacterial cell further comprises an antitoxin under the control of a constitutive promoter. In this situation, in the presence of arabinose, the toxin is not expressed due to repression by TetR protein, and the antitoxin protein builds-up in the cell. However, in the absence of arabinose, TetR protein is not expressed, and expression of the toxin is induced. The toxin begins to build-up within the recombinant bacterial cell. The recombinant bacterial cell is no longer viable once the toxin protein is present at either equal or greater amounts than that of the anti-toxin protein in the cell, and the recombinant bacterial cell will be killed by the toxin.

In another embodiment of the disclosure, the recombinant bacterial cell further comprises an antitoxin under the control of the P_{araBAD} promoter. In this situation, in the presence of arabinose, TetR and the anti-toxin are expressed, the anti-toxin builds up in the cell, and the toxin is not expressed due to repression by TetR protein. However, in the absence of arabinose, both the TetR protein and the anti-toxin are not expressed, and expression of the toxin is induced. The toxin begins to build-up within the recombinant bacterial cell. The recombinant bacterial cell is no longer viable once the toxin protein is expressed, and the recombinant bacterial cell will be killed by the toxin.

In another exemplary embodiment of the disclosure, the engineered bacteria of the present disclosure contains a kill-switch having at least the following sequences: a P_{araBAD} promoter operably linked to a heterologous gene encoding an essential polypeptide not found in the recombinant bacterial cell (and required for survival), and a P_{araC} promoter operably linked to a heterologous gene encoding AraC transcription factor. In the presence of arabinose, the AraC transcription factor activates the P_{araBAD} promoter, which activates transcription of the heterologous gene encoding the essential polypeptide, allowing the recombinant bacterial cell to survive. In the absence of arabinose, however, AraC suppresses transcription from the P_{araBAD} promoter and the essential protein required for survival is not expressed. In this case, the recombinant bacterial cell dies in the absence of arabinose. In some embodiments, the sequence of P_{araBAD} promoter operably linked to a heterologous gene encoding an essential polypeptide not found in the recombinant bacterial cell can be present in the bacterial cell in conjunction with the TetR/toxin kill-switch system described directly above. In some embodiments, the sequence of P_{araBAD} promoter operably linked to a heterologous gene encoding an essential polypeptide not found in the recombinant bacterial cell can be present in the bacterial cell in conjunction with the TetR/toxin/anti-toxin kill-switch system described directly above. In yet other embodiments, the bacteria may comprise a plasmid stability system with a plasmid that produces both a short-lived anti-toxin and a long-lived toxin. In this system, the bacterial cell produces equal amounts of toxin and anti-toxin to neutralize the toxin. However, if/when the cell loses the plasmid, the short-lived anti-toxin begins to decay. When the anti-toxin decays completely the cell dies as a result of the longer-lived toxin killing it.

In some embodiments, the engineered bacteria of the present disclosure, for example, bacteria expressing an amino acid catabolism enzyme further comprise the gene(s) encoding the components of any of the above-described kill-switch circuits.

In any of the above-described embodiments, the bacterial toxin is selected from the group consisting of a lysin, Hok, Fst, TisB, LdrD, Kid, SymE, MazF, FlmA, Ibs, XCV2162, dinJ, CcdB, MazF, ParE, YafO, Zeta, hicB, relB, yhaV, yoeB, chpBK, hipA, microcin B, microcin B17, microcin C, microcin C7-051, microcin J25, microcin ColV, microcin 24, microcin L, microcin D93, microcin L, microcin E492, microcin H47, microcin I47, microcin M, colicin A, colicin E1, colicin K, colicin N, colicin U, colicin B, colicin Ia, colicin Ib, colicin 5, colicin10, colicin S4, colicin Y, colicin E2, colicin E7, colicin E8, colicin E9, colicin E3, colicin E4, colicin E6; colicin E5, colicin D, colicin M, and cloacin DF13, or a biologically active fragment thereof.

In any of the above-described embodiments, the anti-toxin is selected from the group consisting of an anti-lysin, Sok, RNAII, IstR, RdlD, Kis, SymR, MazE, FlmB, Sib, ptaRNA1, yafQ, CcdA, MazE, ParD, yafN, Epsilon, HicA, relE, prlF, yefM, chpBI, hipB, MccE, MccE^{CTD}, MccF, Cai, ImmE1, Cki, Cni, Cui, Cbi, Iia, Imm, Cfi, Im10, Csi, Cyi, Im2, Im7, Im8, Im9, Im3, Im4, ImmE6, cloacin immunity protein (Cim), ImmES, ImmD, and Cmi, or a biologically active fragment thereof.

In one embodiment, the bacterial toxin is bactericidal to the genetically engineered bacterium. In one embodiment, the bacterial toxin is bacteriostatic to the genetically engineered bacterium.

In one embodiment, the method further comprises administering a second recombinant bacterial cell to the subject, wherein the second recombinant bacterial cell comprises a heterologous reporter gene operably linked to an inducible promoter that is directly or indirectly induced by an exogenous environmental condition. In one embodiment, the heterologous reporter gene is a fluorescence gene. In one embodiment, the fluorescence gene encodes a green fluorescence protein (GFP). In another embodiment, the method further comprises administering a second recombinant bacterial cell to the subject, wherein the second recombinant bacterial cell expresses a lacZ reporter construct that cleaves a substrate to produce a small molecule that can be detected in urine (see, for example, Danio et al., Science Translational Medicine, 7(289):1-12, 2015, the entire contents of which are expressly incorporated herein by reference).

Isolated Plasmids

In other embodiments, the disclosure provides an isolated plasmid comprising a first nucleic acid encoding an amino acid catabolism enzyme operably linked to a first inducible promoter. In another embodiment, the disclosure provides an isolated plasmid comprising a second nucleic acid encoding at least one additional amino acid catabolism enzyme. In one embodiment, the first nucleic acid and the second nucleic acid are operably linked to the first promoter. In another embodiment, the second nucleic acid is operably linked to a second inducible promoter. In one embodiment, the first inducible promoter and the second inducible promoter are separate copies of the same inducible promoter. In another embodiment, the first inducible promoter and the second inducible promoter are different inducible promoters. In one embodiment, the first promoter, the second promoter, or the first promoter and the second promoter, are each directly or indirectly induced by low-oxygen or anaerobic conditions. In another embodiment, the first promoter, the second promoter, or the first promoter and the second promoter, are each a fumarate and nitrate reduction regulator (FNR) responsive promoter. In another embodiment, the first promoter, the second promoter, or the first promoter and second promoter are each a ROS-inducible regulatory region. In another embodiment, the first promoter, the second promoter, or the first promoter and second promoter are each a RNS-inducible regulatory region.

In one embodiment, the heterologous gene encoding the amino acid catabolism enzyme is operably linked to a constitutive promoter. In one embodiment, the constitutive promoter is a lac promoter. In another embodiment, the constitutive promoter is a tet promoter. In another embodiment, the constitutive promoter is a constitutive Escherichia coli σ³² promoter. In another embodiment, the constitutive promoter is a constitutive Escherichia coli σ⁷⁰ promoter. In another embodiment, the constitutive promoter is a constitutive Bacillus subtilis σ^{A} promoter. In another embodiment, the constitutive promoter is a constitutive Bacillus subtilis σ^{B} promoter. In another embodiment, the constitutive promoter is a Salmonella promoter. In another embodiment, the constitutive promoter is a bacteriophage T7 promoter. In another embodiment, the constitutive promoter is and a bacteriophage SP6 promoter. In any of the above-described embodiments, the plasmid further comprises a heterologous gene encoding a transporter of an amino acid and/or a kill switch construct, either or both of which may be operably linked to a constitutive promoter or an inducible promoter.

In one embodiment, the isolated plasmid comprises at least one heterologous gene encoding an amino acid catabolism enzyme operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a P_{TetR} promoter. In another embodiment, the isolated plasmid comprises at least one heterologous gene encoding an amino acid catabolism enzyme operably linked to a first inducible promoter; a heterologous gene encoding a TetR protein and an anti toxin operably linked to a P_{araBAD} promoter, a heterologous gene encoding AraC operably linked to a P_{araC} promoter, and a heterologous gene encoding a toxin operably linked to a P_{TetR} promoter.

In any of the above-described embodiments, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

C. Constitutive Promoters

In some embodiments, the gene encoding the payload is present on a plasmid and operably linked to a constitutive promoter. In some embodiments, the gene encoding the payload is present on a chromosome and operably linked to a constitutive promoter.

In some embodiments, the constitutive promoter is active under in vivo conditions, e.g., the gut, or in the presence of metabolites associated with certain bile salt diseases, as described herein. In some embodiments, the promoter is active under in vitro conditions, e.g., various cell culture and/or cell manufacturing conditions, as described herein. In some embodiments, the constitutive promoter is active under in vivo conditions, e.g., the gut and/or in the presence of metabolites associated with certain diseases, such as bile salt associated diseases and conditions, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions).

In some embodiments, the constitutive promoter is active in exogenous environmental conditions specific to the gut of a mammal. In some embodiments, the constitutive promoter is active in exogenous environmental conditions specific to the small intestine of a mammal. In some embodiments, the constitutive promoter is active in low-oxygen or anaerobic conditions such as the environment of the mammalian gut. In some embodiments, the constitutive promoter is active in the presence of molecules or metabolites that are specific to the gut of a mammal. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions.

Bacterial constitutive promoters are known in the art. Examplary constitutive promoters are listed in the following Tables. The strength of the constitutive promoter can be further fine-tuned through the selection of ribosome binding sites of the desired strengths.

In some embodiments, the gene sequence(s) encoding an amino acid catabolism enzyme is operably linked to a Escherichia coli σ70 promoter. Exemplary E. coli σ70 promoters are listed in Table 8.

In some embodiments, the gene sequence(s) encoding an amino acid catabolism enzyme is operably linked to a Escherichia coli σ70 promoter. Exemplary E. coli σ70 promoters are listed in Table 6A.

TABLE 8
Constitutive E. coli σ70 promoters

| SEQ | Name | Description | Promoter Sequence | Length |
|---|---|---|---|---|
| SEQ ID NO: | BBa_I14018 | P(Bla) | ... | 35 |
| 152 | | | gtttatacataggcgagtactctgttatgg | |
| SEQ ID NO: | BBa_I14033 | P(Cat) | ... | 38 |
| 153 | | | agaggttccaactacaccataatgaaaca | |
| SEQ ID NO: | BBa_I14034 | P(Kat) | ... | 45 |
| 154 | | | taaacaactaacggacaattctacctaaca | |
| SEQ ID NO: | BBa_I732021 | Template for Building | ... | 159 |
| 155 | | Primer Family Member | acatcaagccaaattaaacaggattaacac | |
| SEQ ID NO: | BBa_I742126 | Reverse lambda cI- | ... | 49 |
| 156 | | regulated promoter | gaggtaaaatagtcaacacgcacggtgtta | |
| SEQ ID NO: | BBa_J01006 | Key Promoter absorbs 3 | ... | 59 |
| 157 | | | caggccggaataactccctataatgcgcca | |
| SEQ ID NO: | BBa_J23100 | constitutive promoter | ... | 35 |
| 158 | | family member | ggctagctcagtcctaggtacagtgctagc | |
| SEQ ID NO: | BBa_J23101 | constitutive promoter | ... | 35 |
| 159 | | family member | agctagctcagtcctaggtattatgctagc | |
| SEQ ID NO: | BBa_J23102 | constitutive promoter | ... | 35 |
| 160 | | family member | agctagctcagtcctaggtactgtgctagc | |
| SEQ ID NO: | BBa_J23103 | constitutive promoter | ... | 35 |
| 161 | | family member | agctagctcagtcctagggattatgctagc | |
| SEQ ID NO: | BBa_J23104 | constitutive promoter | ... | 35 |
| 162 | | family member | agctagctcagtcctaggtattgtgctagc | |
| SEQ ID NO: | BBa_J23105 | constitutive promoter | ... | 35 |
| 163 | | family member | ggctagctcagtcctaggtactatgctagc | |
| SEQ ID NO: | BBa_J23106 | constitutive promoter | ... | 35 |
| 164 | | family member | ggctagctcagtcctaggtatagtgctagc | |
| SEQ ID NO: | BBa_J23107 | constitutive promoter | ... | 35 |
| 165 | | family member | ggctagctcagccctaggtattatgctagc | |
| SEQ ID NO: | BBa_J23108 | constitutive promoter | ... | 35 |
| 166 | | family member | agctagctcagtcctaggtataatgctagc | |
| SEQ ID NO: | BBa_J23109 | constitutive promoter | ... | 35 |
| 167 | | family member | agctagctcagtcctagggactgtgctagc | |
| SEQ ID NO: | BBa_J23110 | constitutive promoter | ... | 35 |
| 168 | | family member | ggctagctcagtcctaggtacaatgctagc | |
| SEQ ID NO: | BBa_J23111 | constitutive promoter | ... | 35 |
| 169 | | family member | ggctagctcagtcctaggtatagtgctagc | |
| SEQ ID NO: | BBa_J23112 | constitutive promoter | ... | 35 |
| 170 | | family member | agctagctcagtcctagggattatgctagc | |
| SEQ ID NO: | BBa_J23113 | constitutive promoter | ... | 35 |
| 171 | | family member | ggctagctcagtcctagggattatgctagc | |
| SEQ ID NO: | BBa_J23114 | constitutive promoter | ... | 35 |
| 172 | | family member | ggctagctcagtcctaggtacaatgctagc | |
| SEQ ID NO: | BBa_J23115 | constitutive promoter | ... | 35 |
| 173 | | family member | agctagctcagcccttggtacaatgctagc | |
| SEQ ID NO: | BBa_J23116 | constitutive promoter | ... | 35 |
| 174 | | family member | agctagctcagtcctagggactatgctagc | |
| SEQ ID NO: | BBa_J23117 | constitutive promoter | ... | 35 |
| 175 | | family member | agctagctcagtcctagggattgtgctagc | |
| SEQ ID NO: | BBa_J23118 | constitutive promoter | ... | 35 |
| 176 | | family member | ggctagctcagtcctaggtattgtgctagc | |
| SEQ ID NO: | BBa_J23119 | constitutive promoter | ... | 35 |
| 177 | | family member | agctagctcagtcctaggtataatgctagc | |
| SEQ ID NO: | BBa_J23150 | 1bp mutant from J23107 | ... | 35 |
| 178 | | | ggctagctcagtcctaggtattatgctagc | |
| SEQ ID NO: | BBa_J23151 | 1bp mutant from J23114 | ... | 35 |
| 179 | | | ggctagctcagtcctaggtacaatgctagc | |
| SEQ ID NO: | BBa_J44002 | pBAD reverse | ... | 130 |
| 180 | | | aaagtgtgacgccgtgcaaataatcaatgt | |
| SEQ ID NO: | BBa_J48104 | NikR promoter, a protein | ... | 40 |
| 181 | | of the ribbon helix-helix | gacgaatacttaaaatcgtcatacttattt | |
| | | family of trancription | | |
| | | factors that repress expre | | |
| SEQ ID NO: | BBa_J54200 | lacq_Promoter | ... | 50 |
| 182 | | | aaacctttcgcggtatggcatgatagcgcc | |
| SEQ ID NO: | BBa_J56015 | lacIQ - promoter sequence | ... | 57 |
| 183 | | | tgatagcgcccggaagagagtcaattcagg | |
| SEQ ID NO: | BBa_J64951 | E. Coli CreABCD | ... | 81 |
| 184 | | phosphate sensing operon | ttatttaccgtgacgaactaattgctcgtg | |
| | | promoter | | |
| SEQ ID NO: | BBa_K088007 | GlnRS promoter | ... | 38 |
| 185 | | | catacgccgttatacgttgtttacgctttg | |
| SEQ ID NO: | BBa_K119000 | Constitutive weak | ... | 38 |
| 186 | | promoter of lacZ | ttatgcttccggctcgtatgagtgtggac | |
| SEQ ID NO: | BBa_K119001 | Mutated LacZ promoter | ... | 38 |
| 187 | | | ttatgcttccggctcgtatggtgtgtggac | |
| SEQ ID NO: | BBa_K1330002 | Constitutive promoter | ... | 35 |
| 188 | | (J23105) | ggctagctcagtcctaggtactatgctagc | |
| SEQ ID NO: | BBa_K137029 | constitutive promoter with | ... atatatatatatatataatggaagcgtttt | 39 |
| 189 | | (TA)10 between −10 and −35 | | |
| | | elements | | |
| SEQ ID NO: | BBa_K137030 | constitutive promoter with | ... atatatatatatatataatggaagcgtttt | 37 |
| 190 | | (TA)9 between −10 and −35 | | |
| | | elements | | |
| SEQ ID NO: | BBa_K137031 | constitutive promoter with | ... | 62 |
| 191 | | (C)10 between −10 and −35 | ccccgaaagcttaagaatataattgtaagc | |
| | | elements | | |
| SEQ ID NO: | BBa_K137032 | constitutive promoter with | ... | 64 |
| 192 | | (C)12 between −10 and −35 | ccccgaaagcttaagaatataattgtaagc | |
| | | elements | | |
| SEQ ID NO: | BBa_K137085 | optimized (TA) repeat | ... | 31 |
| 193 | | constitutive promoter with | tgacaatatatatatatatataatgctagc | |
| | | 13 bp between −10 and −35 | | |
| | | elements | | |
| SEQ ID NO: | BBa_K137086 | optimized (TA) repeat | ... | 33 |
| 194 | | constitutive promoter with | acaatatatatatatatatataatgctagc | |
| | | 15 bp between −10 and −35 | | |
| | | elements | | |
| SEQ ID NO: | BBa_K137087 | optimized (TA) repeat | ... aatatatatatatatatatataatgctagc | 35 |
| 195 | | constitutive promoter with | | |
| | | 17 bp between −10 and −35 | | |
| | | elements | | |
| SEQ ID NO: | BBa_K137088 | optimized (TA) repeat | ... tatatatatatatatatatataatgctagc | 37 |
| 196 | | constitutive promoter with | | |
| | | 19 bp between −10 and −35 | | |
| | | elements | | |
| SEQ ID NO: | BBa_K137089 | optimized (TA) repeat | ... tatatatatatatatatatataatgctagc | 39 |
| 197 | | constitutive promoter with | | |
| | | 21 bp between −10 and −35 | | |
| | | elements | | |
| SEQ ID NO: | BBa_K137090 | optimized (A) repeat | ... | 35 |
| 198 | | constitutive promoter with | aaaaaaaaaaaaaaaaaatataatgctagc | |
| | | 17 bp between −10 and −35 | | |
| | | elements | | |
| SEQ ID NO: | BBa_K137091 | optimized (A) repeat | ... | 36 |
| 199 | | constitutive promoter with | aaaaaaaaaaaaaaaaaatataatgctagc | |
| | | 18 bp between −10 and −35 | | |
| | | elements | | |
| SEQ ID NO: | BBa_K1585100 | Anderson Promoter with | ... | 78 |
| 200 | | lacI binding site | ggaattgtgagcggataacaatttcacaca | |
| SEQ ID NO: | BBa_K1585101 | Anderson Promoter with | ... | 78 |
| 201 | | lacI binding site | ggaattgtgagcggataacaatttcacaca | |
| SEQ ID NO: | BBa_K1585102 | Anderson Promoter with | ... | 78 |
| 202 | | lacI binding site | ggaattgtgagcggataacaatttcacaca | |
| SEQ ID NO: | BBa_K1585103 | Anderson Promoter with | ... | 78 |
| 203 | | lacI binding site | ggaattgtgagcggataacaatttcacaca | |
| SEQ ID NO: | BBa_K1585104 | Anderson Promoter with | ... | 78 |
| 204 | | lacI binding site | ggaattgtgagcggataacaatttcacaca | |
| SEQ ID NO: | BBa_K1585105 | Anderson Promoter with | ... | 78 |
| 205 | | lacI binding site | ggaattgtgagcggataacaatttcacaca | |
| SEQ ID NO: | BBa_K1585106 | Anderson Promoter with | ... | 78 |
| 206 | | lacI binding site | ggaattgtgagcggataacaatttcacaca | |
| SEQ ID NO: | BBa_K1585110 | Anderson Promoter with | ... | 78 |
| 207 | | lacI binding site | ggaattgtgagcggataacaatttcacaca | |
| SEQ ID NO: | BBa_K1585113 | Anderson Promoter with | ... | 78 |
| 208 | | lacI binding site | ggaattgtgagcggataacaatttcacaca | |
| SEQ ID NO: | BBa_K1585115 | Anderson Promoter with | ... | 78 |
| 209 | | lacI binding site | ggaattgtgagcggataacaatttcacaca | |
| SEQ ID NO: | BBa_K1585116 | Anderson Promoter with | ... | 78 |
| 210 | | lacI binding site | ggaattgtgagcggataacaatttcacaca | |
| SEQ ID NO: | BBa_K1585117 | Anderson Promoter with | ... | 78 |
| 211 | | lacI binding site | ggaattgtgagcggataacaatttcacaca | |
| SEQ ID NO: | BBa_K1585118 | Anderson Promoter with | ... | 78 |
| 212 | | lacI binding site | ggaattgtgagcggataacaatttcacaca | |
| SEQ ID NO: | BBa_K1585119 | Anderson Promoter with | ... | 78 |
| 213 | | lacI binding site | ggaattgtgagcggataacaatttcacaca | |
| SEQ ID NO: | BBa_K1824896 | J23100 + RBS | ... | 88 |
| 214 | | | gattaaagaggagaaatactagagtactag | |
| SEQ ID NO: | BBa_K256002 | J23101:GFP | ... | 918 |
| 215 | | | caccttcgggtgggccatctgcgtttata | |
| SEQ ID NO: | BBa_K256018 | J23119:IFP | ... | 1167 |
| 216 | | | caccttcgggtgggccatctgcgtttata | |
| SEQ ID NO: | BBa_K256020 | J23119:HO1 | ... | 949 |
| 217 | | | caccttcgggtgggccatctgcgtttata | |
| SEQ ID NO: | BBa_K256033 | Infrared signal reporter | ... | 2124 |
| 218 | | (J23119:IFP:J23119:HO1) | caccttcgggtgggccatctgcgtttata | |
| SEQ ID NO: | BBa_K292000 | Double terminator + | ... | 138 |
| 219 | | constitutive promoter | ggctagctcagtcctaggtacagtgctagc | |
| SEQ ID NO: | BBa_K292001 | Double terminator + | ... | 161 |
| 220 | | Constitutive promoter + | tgctagctactagagattaaagaggagaaa | |
| | | Strong RBS | | |
| SEQ ID NO: | BBa_K418000 | IPTG inducible Lac | ... | 1416 |
| 221 | | promoter cassette | ttgtgagcggataacaagatactgagcaca | |
| SEQ ID NO: | BBa_K418002 | IPTG inducible Lac | ... | 1414 |
| 222 | | promoter cassette | ttgtgagcggataacaagatactgagcaca | |
| SEQ ID NO: | BBa_K418003 | IPTG inducible Lac | ... | 1416 |
| 223 | | promoter cassette | ttgtgagcggataacaagatactgagcaca | |
| SEQ ID NO: | BBa_K823004 | Anderson promoter | ... | 35 |
| 224 | | J23100 | ggctagctcagtcctaggtacagtgctagc | |
| SEQ ID NO: | BBa_K823005 | Anderson promoter | ... | 35 |
| 225 | | J23101 | agctagctcagtcctaggtattatgctagc | |
| SEQ ID NO: | BBa_K823006 | Anderson promoter | ... | 35 |
| 226 | | J23102 | agctagctcagtcctaggtactgtgctagc | |
| SEQ ID NO: | BBa_K823007 | Anderson promoter | ... | 35 |
| 227 | | J23103 | agctagctcagtcctagggattatgctagc | |
| SEQ ID NO: | BBa_K823008 | Anderson promoter | ... | 35 |
| 228 | | J23106 | ggctagctcagtcctaggtatagtgctagc | |
| SEQ ID NO: | BBa_K823010 | Anderson promoter | ... | 35 |
| 229 | | J23113 | ggctagctcagtcctagggattatgctagc | |
| SEQ ID NO: | BBa_K823011 | Anderson promoter | ... | 35 |
| 230 | | J23114 | ggctagctcagtcctaggtacaatgctagc | |
| SEQ ID NO: | BBa_K823013 | Anderson promoter | ... | 35 |
| 231 | | J23117 | agctagctcagtcctagggattgtgctagc | |
| SEQ ID NO: | BBa_K823014 | Anderson promoter | ... | 35 |
| 232 | | J23118 | ggctagctcagtcctaggtattgtgctagc | |
| SEQ ID NO: | BBa_M13101 | M13K07 gene I promoter | ... cctgatttatgttattctctctgtaaagg | 47 |
| 233 | | | | |
| SEQ ID NO: | BBa_M13102 | M13K07 gene II promoter | ... aaatatttgcttatacaatcttcctgtttt | 48 |
| 234 | | | | |
| SEQ ID NO: | BBa_M13103 | M13K07 gene III | ... | 48 |
| 235 | | promoter | gctgataaaccgatacaattaaaggctcct | |
| SEQ ID NO: | BBa_M13104 | M13K07 gene IV | ... | 49 |
| 236 | | promoter | ctcttctcagcgtcttaatctaagctatcg | |
| SEQ ID NO: | BBa_M13105 | M13K07 gene V promoter | ... | 50 |
| 237 | | | atgagccagttcttaaaatcgcataaggta | |
| SEQ ID NO: | BBa_M13106 | M13K07 gene VI | ... | 49 |
| 238 | | promoter | ctattgattgtgacaaaataaacttattcc | |
| SEQ ID NO: | BBa_M13108 | M13K07 gene VIII | ... | 47 |
| 239 | | promoter | gtttcgcgcttggtataatcgctgggggtc | |
| SEQ ID NO: | BBa_M13110 | M13110 | ... | 48 |
| 240 | | | ctttgcttctgactataatagtcagggtaa | |
| SEQ ID NO: | BBa_M31519 | Modified promoter | ... | 60 |
| 241 | | sequence of g3. | aaaccgatacaattaaaggctcctgctagc | |
| SEQ ID NO: | BBa_R1074 | Constitutive Promoter I | ... | 74 |
| 242 | | | caccacactgatagtgctagtgtagatcac | |
| SEQ ID NO: | BBa_R1075 | Constitutive Promoter II | ... | 49 |
| 243 | | | gccggaataactccctataatgcgccacca | |
| SEQ ID NO: | BBa_S03331 | --Specify Parts List-- | ttgacaagcttttcctcagctccgtaaact | |
| 244 | | | | |

In some embodiments, the gene sequence(s) encoding an amino acid catabolism enzyme is operably linked to a E. coli σS promoters. Exemplary E. coli σS promoters are listed in Table 9.

TABLE 9
Constitutive E. coli σ^{S} promoters

| SEQ | Name | Description | Promoter Sequence | Length |
|---|---|---|---|---|
| SEQ ID NO: | BBa_J45992 | Full-length stationary | ...ggtttcaaaattgtgatctatatttaacaa | 199 |
| 245 | | phase osmY promoter | | |
| SEQ ID NO: | BBa_J45993 | Minimal stationary | ...ggtttcaaaattgtgatctatatttaacaa | 57 |
| 246 | | phase osmY promoter | | |

In some embodiments, the gene sequence(s) encoding an amino acid catabolism enzyme and/or bile salt and/or bile acid transporter, and/or bile salt and/or bile acid exporter is operably linked to a E. coli σ³² promoters. Exemplary E. coli σ³² promoters are listed in Table 10.

TABLE 10
Constitutive E. coli σ³² promoters

| SEQ | Name | Description | Promoter Sequence | Length |
|---|---|---|---|---|
| SEQ ID NO: 247 | BBa_J45504 | htpG Heat Shock | ...tctattccaataaagaaatcttcctgcgtg | 405 |
| | | Promoter | | |
| SEQ ID NO: 248 | BBa_K1895002 | dnaK Promoter | ... | 182 |
| | | | gaccgaatatatagtggaaacgtttagatg | |
| SEQ ID NO: 249 | BBa_K1895003 | htpG Promoter | ...ccacatcctgtttttaaccttaaaatggca | 287 |

In some embodiments, the gene sequence(s) encoding amino acid catabolism enzyme and/or bile salt and/or bile acid transporter, and/or bile salt and/or bile acid exporter is operably linked to a B. subtilis σ^{A} promoters. Exemplary B. subtilis σ^{A} promoters are listed in Table 11.

TABLE 11
Constitutive B. subtilis σ^{A} promoters

| SEQ | Name | Description | Promoter Sequence | Length |
|---|---|---|---|---|
| SEQ ID NO: 250 | BBa_K143012 | Promoter veg a | ... | 97 |
| | | constitutive promoter | aaaaatgggctcgtgttgtacaataaatgt | |
| | | for B. subtilis | | |
| SEQ ID NO: 251 | BBa_K143013 | Promoter 43 a | ... | 56 |
| | | constitutive promoter | aaaaaaagcgcgcgattatgtaaaatataa | |
| | | for B. subtilis | | |
| SEQ ID NO: 252 | BBa_K780003 | Strong constitutive | ... | 36 |
| | | promoter for Bacillus | aattgcagtaggcatgacaaaatggactca | |
| | | subtilis | | |
| SEQ ID NO: 253 | BBa_K823000 | P_{liaG} | ... | 121 |
| | | | caagcnttcattataatagaatgaatga | |
| SEQ ID NO: 254 | BBa_K823002 | P_{lepA} | ...tctaagctagtgtattttgcgtttaatagt | 157 |
| SEQ ID NO: 255 | BBa_K823003 | P_{veg} | ... | 237 |
| | | | aatgggctcgtgttgtacaataaatgtagt | |

In some embodiments, the gene sequence(s) encoding amino acid catabolism enzyme and/or bile salt and/or bile acid transporter, and/or bile salt and/or bile acid exporter is operably linked to a B. subtilis σB promoters. Exemplary B. subtilis σB promoters are listed in Table 12.

TABLE 12
Constitutive B. subtilis σ^{B} promoters

| SEQ | Name | Description | Promoter Sequence | Length |
|---|---|---|---|---|
| SEQ ID NO: 256 | BBa_K143010 | Promoter ctc for | ...atccttatcgttatgggtattgtttgtaat | 56 |
| B. subtilis | | | | |
| SEQ ID NO: 257 | BBa_K143011 | Promoter gsiB for | ... | 38 |
| | | B. subtilis | taaaagaattgtgagcgggaatacaacaac | |
| SEQ ID NO: 258 | BBa_K143013 | Promoter 43 a | ... | 56 |
| | | constitutive promoter | aaaaaaagcgcgcgattatgtaaaatataa | |
| | | for B. subtilis | | |

In some embodiments, the gene sequence(s) encoding an amino acid catabolism enzyme is operably linked to promoters from Salmonella. Exemplary Salmonella promoters are listed in Table 13.

TABLE 13
Constitutive promoters from miscellaneous prokaryotes

| SEQ | Name | Description | Promoter Sequence | Length |
|---|---|---|---|---|
| SEQ ID NO: 259 | BBa_K112706 | Pspv2 | ... | 474 |
| | | from Salmonella | tacaaaataattcccctgcaaacattatca | |
| SEQ ID NO: 260 | BBa_K112707 | Pspv from Salmonella | ... | 1956 |
| | | | tacaaaataattcccctgcaaacattatcg | |

In some embodiments, the gene sequence(s) encoding an amino acid catabolism enzyme is operably linked to promoters from bacteriophage T7. Exemplary promoters from bacteriophage T7 are listed in Table 14.

TABLE 14
Constitutive promoters from bacteriophage T7

| SEQ | Name | Description | Promoter Sequence | Length |
|---|---|---|---|---|
| SEQ ID NO: 261 | BBa_I712074 | T7 promoter (strong | ... | 46 |
| | | promoter from T7 | agggaatacaagctacttgttctttttgca | |
| | | bacteriophage) | | |
| SEQ ID NO: 262 | BBa_I719005 | T7 Promoter | taatacgactcactatagggaga | 23 |
| SEQ ID NO: 263 | BBa_J34814 | T7 Promoter | gaatttaatacgactcactatagggaga | 28 |
| SEQ ID NO: 264 | BBa_J64997 | T7 consensus −10 and | taatacgactcactatagg | 19 |
| | | rest | | |
| SEQ ID NO: 265 | BBa_K113010 | overlapping T7 | ... | 40 |
| | | promoter | gagtcgtattaatacgactcactatagggg | |
| SEQ ID NO: 266 | BBa_K113011 | more overlapping T7 | ... | 37 |
| | | promoter | agtgagtcgtactacgactcactatagggg | |
| SEQ ID NO: 267 | BBa_K113012 | weaken overlapping | ... | 40 |
| | | T7 promoter | gagtcgtattaatacgactctctatagggg | |
| SEQ ID NO: 268 | BBa_K1614000 | T7 promoter for | taatacgactcactatag | 18 |
| | | expression of | | |
| | | functional RNA | | |
| SEQ ID NO: 269 | BBa_R0085 | T7 Consensus | taatacgactcactatagggaga | 23 |
| | | Promoter Sequence | | |
| SEQ ID NO: 270 | BBa_R0180 | T7 RNAP promoter | ttatacgactcactatagggaga | 23 |
| SEQ ID NO: 271 | BBa_R0181 | T7 RNAP promoter | gaatacgactcactatagggaga | 23 |
| SEQ ID NO: 272 | BBa_R0182 | T7 RNAP promoter | taatacgtctcactatagggaga | 23 |
| SEQ ID NO: 273 | BBa_R0183 | T7 RNAP promoter | tcatacgactcactatagggaga | 23 |
| SEQ ID NO: 274 | BBa_Z0251 | T7 strong promoter | ... | 35 |
| | | | taatacgactcactatagggagaccacaac | |
| SEQ ID NO: 275 | BBa_Z0252 | T7 weak binding and | ... | 35 |
| | | processivity | taattgaactcactaaagggagaccacagc | |
| SEQ ID NO: 276 | BBa_Z0253 | T7 weak binding | ... | 35 |
| | | promoter | cgaagtaatacgactcactattagggaaga | |

In some embodiments, the gene sequence(s) encoding an amino acid catabolism enzyme is operably linked to promoters bacteriophage SP6. Exemplary promoters from bacteriophage SP6 are listed in Table 15.

TABLE 15
Constitutive promoters from bacteriophage SP6

| | | | Promoter | |
|---|---|---|---|---|
| SEQ | Name | Description | Sequence | Length |
| SEQ ID | BBa_J64998 | consensus −10 | atttaggtgac | 19 |
| NO: 277 | | and rest from | actataga | |
| | | SP6 | | |

In some embodiments, the gene sequence(s) encoding an amino acid catabolism enzyme is operably linked to promoters from yeast. Exemplary promoters from yeast are listed in Table 16.

TABLE 16
Constitutive promoters from yeast

| SEQ | Name | Description | Promoter Sequence | Length |
|---|---|---|---|---|
| SEQ ID NO: 278 | BBa_I766555 | pCyc (Medium) | ... | 244 |
| | | Promoter | acaaacacaaatacacacactaaattaata | |
| SEQ ID NO: 279 | BBa_I766556 | pAdh (Strong) Promoter | ... | 1501 |
| | | | ccaagcatacaatcaactatctcatataca | |
| SEQ ID NO: 280 | BBa_I766557 | pSte5 (Weak) Promoter | ... | 601 |
| | | | gatacaggatacagcggaaacaacttttaa | |
| SEQ ID NO: 281 | BBa_J63005 | yeast ADH1 promoter | ... | 1445 |
| | | | tttcaagctataccaagcatacaatcaact | |
| SEQ ID NO: 282 | BBa_K105027 | cyc100 minimal | ...cctttgcagcataaattactatacttctat | 103 |
| | | promoter | | |
| SEQ ID NO: 283 | BBa_K105028 | cyc70 minimal | ...cctttgcagcataaattactatacttctat | 103 |
| | | promoter | | |
| SEQ ID NO: 284 | BBa_K105029 | cyc43 minimal | ...cctttgcagcataaattactatacttctat | 103 |
| | | promoter | | |
| SEQ ID NO: 285 | BBa_K105030 | cyc28 minimal | ...cctttgcagcataaattactatacttctat | 103 |
| | | promoter | | |
| SEQ ID NO: 286 | BBa_K105031 | cyc16 minimal | ...cctttgcagcataaattactatacttctat | 103 |
| | | promoter | | |
| SEQ ID NO: 287 | BBa_K122000 | pPGK1 | ...ttatctactttttacaacaaatataaaaca | 1497 |
| SEQ ID NO: 288 | BBa_K124000 | pCYC Yeast Promoter | ... | 288 |
| | | | acaaacacaaatacacacactaaattaata | |
| SEQ ID NO: 289 | BBa_K124002 | Yeast GPD (TDH3) | ... | 681 |
| | | Promoter | gtttcgaataaacacacataaacaaacaaa | |
| SEQ ID NO: 290 | BBa_K319005 | yeast mid-length ADH1 | ... | 720 |
| | | promoter | ccaagcatacaatcaactatctcatataca | |
| SEQ ID NO: 291 | BBa_M31201 | Yeast CLB1 promoter | ... | 500 |
| | | region, G2/M cell cycle | accatcaaaggaagattaatcnctcata | |
| | | specific | | |

In some embodiments, the gene sequence(s) encoding a amino acid catabolism enzyme is operably linked to promoters from eukaryotes. Exemplary promoters from eukaryotes are listed in Table 17.

TABLE 17
Constitutive promoters from miscellaneous eukaryotes

| SEQ | Name | Description | Promoter Sequence | Length |
|---|---|---|---|---|
| SEQ ID NO: 292 | BBa_I712004 | CMV promoter | ...agaacccactgcttactggcttatcgaaat | 654 |
| SEQ ID NO: 293 | BBa_K076017 | Ubc Promoter | ...ggccgtttttggcttttttgttagacgaag | 1219 |

Other exemplary promoters are listed in Table 18.

TABLE 18
Other Constitutive Promoters

| SEQ | Name | Sequence | Description |
|---|---|---|---|
| SEQ ID | Plpp | ataagtgccttcccatcaaaaaaatatt | The Plpp promoter is a natural promoter |
| NO: 294 | | ctcaacataaaaaactttgtgtaatactt | taken from the Nissle genome. In situ it |
| | | gtaacgcta | is used to drive production of lpp, which |
| | | | is known to be the most abundant protein |
| | | | in the cell. Also, in some previous |
| | | | RNAseq experiments I was able to |
| | | | confirm that the lpp mRNA is one of the |
| | | | most abundant mRNA in Nissle during |
| | | | exponential growth. |
| SEQ ID | PapFAB46 | AAAAAGAGTATTGACT | See, e.g., Kosuri, S., Goodman, D. B. & |
| NO: 295 | | TCGCATCTTTTTGTACC | Cambray, G. Composability of |
| | | TATAATAGATTCATTGC | regulatory sequences controlling |
| | | TA | transcription and translation in |
| | | | Escherichia coli. in 1-20 (2013). |
| | | | doi: 10.1073/pnas. |
| SEQ ID | PJ23101 + | ggaaaatttttttaaaaaaaaaactttac | UP element helps recruit RNA |
| NO: 296 | UP | agctagctcagtcctaggtattatgcta | polymerase |
| | element | gc | (ggaaaatttttttaaaaaaaaaac) |
| SEQ ID | PJ23107 + | ggaaaatttttttaaaaaaaaaactttac | UP element helps recruit RNA |
| NO: 297 | UP | ggctagctcagccctaggtattatgct | polymerase |
| | element | agc | (ggaaaatttttttaaaaaaaaaac) |
| SEQ ID | PSYN23119 | ggaaaatttttttaaaaaaaaaacTT | UP element at 5′ end; consensus −10 |
| NO: 298 | | GACAGCTAGCTCAGTC | region is TATAAT; the consensus −35 is |
| | | CTTGGTATAATGCTAG | TTGACA; the extended −10 region is |
| | | CACGAA | generally TGNTATAAT |
| | | | (TGGTATAAT in this sequence) |

In some embodiments, the constitutive promoter is at least about 80%, at least about 85%, at least about 90%, at least about 95%, or at least about 99% homologous to the sequence of SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, and/or SEQ ID NO: 298.

Host-Plasmid Mutual Dependency

In some embodiments, the genetically engineered bacteria also comprise a plasmid that has been modified to create a host-plasmid mutual dependency. In certain embodiments, the mutually dependent host-plasmid platform is GeneGuard (Wright et al., 2015). In some embodiments, the GeneGuard plasmid comprises (i) a conditional origin of replication, in which the requisite replication initiator protein is provided in trans; (ii) an auxotrophic modification that is rescued by the host via genomic translocation and is also compatible for use in rich media; and/or (iii) a nucleic acid sequence which encodes a broad-spectrum toxin. The toxin gene may be used to select against plasmid spread by making the plasmid DNA itself disadvantageous for strains not expressing the anti-toxin (e.g., a wild-type bacterium). In some embodiments, the GeneGuard plasmid is stable for at least 100 generations without antibiotic selection. In some embodiments, the GeneGuard plasmid does not disrupt growth of the host. The GeneGuard plasmid is used to greatly reduce unintentional plasmid propagation in the genetically engineered bacteria described herein.

The mutually dependent host-plasmid platform may be used alone or in combination with other biosafety mechanisms, such as those described herein (e.g., kill switches, auxotrophies). In some embodiments, the genetically engineered bacteria comprise a GeneGuard plasmid. In other embodiments, the genetically engineered bacteria comprise a GeneGuard plasmid and/or one or more kill switches. In other embodiments, the genetically engineered bacteria comprise a GeneGuard plasmid and/or one or more auxotrophies. In still other embodiments, the genetically engineered bacteria comprise a GeneGuard plasmid, one or more kill switches, and/or one or more auxotrophies.

In some embodiments, the vector comprises a conditional origin of replication. In some embodiments, the conditional origin of replication is a R6K or ColE2-P9. In embodiments where the plasmid comprises the conditional origin of replication R6K, the host cell expresses the replication initiator protein π. In embodiments where the plasmid comprises the conditional origin or replication ColE2, the host cell expresses the replication initiator protein RepA. It is understood by those of skill in the art that the expression of the replication initiator protein may be regulated so that a desired expression level of the protein is achieved in the host cell to thereby control the replication of the plasmid. For example, in some embodiments, the expression of the gene encoding the replication initiator protein may be placed under the control of a strong, moderate, or weak promoter to regulate the expression of the protein.

In some embodiments, the vector comprises a gene encoding a protein required for complementation of a host cell auxotrophy, preferably a rich-media compatible auxotrophy. In some embodiments, the host cell is auxotrophic for thymidine (ΔthyA), and the vector comprises the thymidylate synthase (thyA) gene. In some embodiments, the host cell is auxotrophic for diaminopimelic acid (ΔdapA) and the vector comprises the 4-hydroxy-tetrahydrodipicolinate synthase (dapA) gene. It is understood by those of skill in the art that the expression of the gene encoding a protein required for complementation of the host cell auxotrophy may be regulated so that a desired expression level of the protein is achieved in the host cell.

In some embodiments, the vector comprises a toxin gene. In some embodiments, the host cell comprises an anti-toxin gene encoding and/or required for the expression of an anti-toxin. In some embodiments, the toxin is Zeta and the anti-toxin is Epsilon. In some embodiments, the toxin is Kid, and the anti-toxin is Kis. In preferred embodiments, the toxin is bacteriostatic. Any of the toxin/antitoxin pairs described herein may be used in the vector systems of the present disclosure. It is understood by those of skill in the art that the expression of the gene encoding the toxin may be regulated using art known methods to prevent the expression levels of the toxin from being deleterious to a host cell that expresses the anti-toxin. For example, in some embodiments, the gene encoding the toxin may be regulated by a moderate promoter. In other embodiments, the gene encoding the toxin may be cloned adjacent to ribosomal binding site of interest to regulate the expression of the gene at desired levels (see, e.g., Wright et al. (2015)).

Integration

In some embodiments, any of the gene(s) or gene cassette(s) of the present disclosure may be integrated into the bacterial chromosome at one or more integration sites. One or more copies of the gene (for example, an amino acid catabolism gene) or gene cassette (for example, a gene cassette comprising an amino acid catabolism gene and an amino acid transporter gene) may be integrated into the bacterial chromosome. Having multiple copies of the gene or gene cassette integrated into the chromosome allows for greater production of the amino acid catabolism enzyme, and other enzymes of the gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

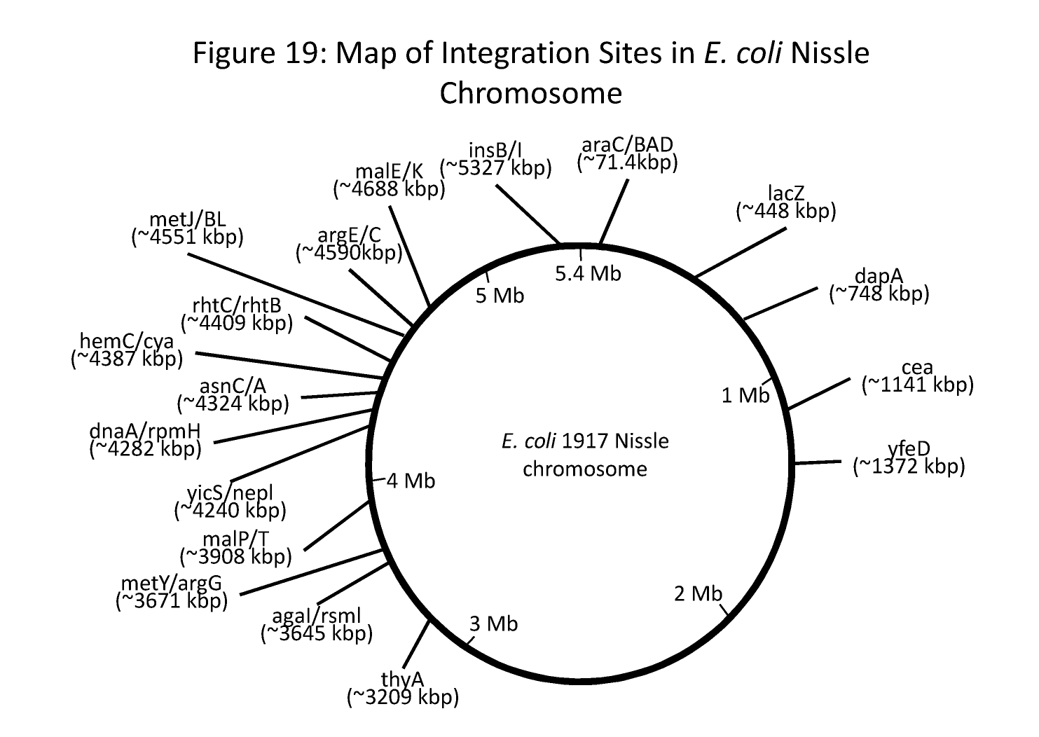

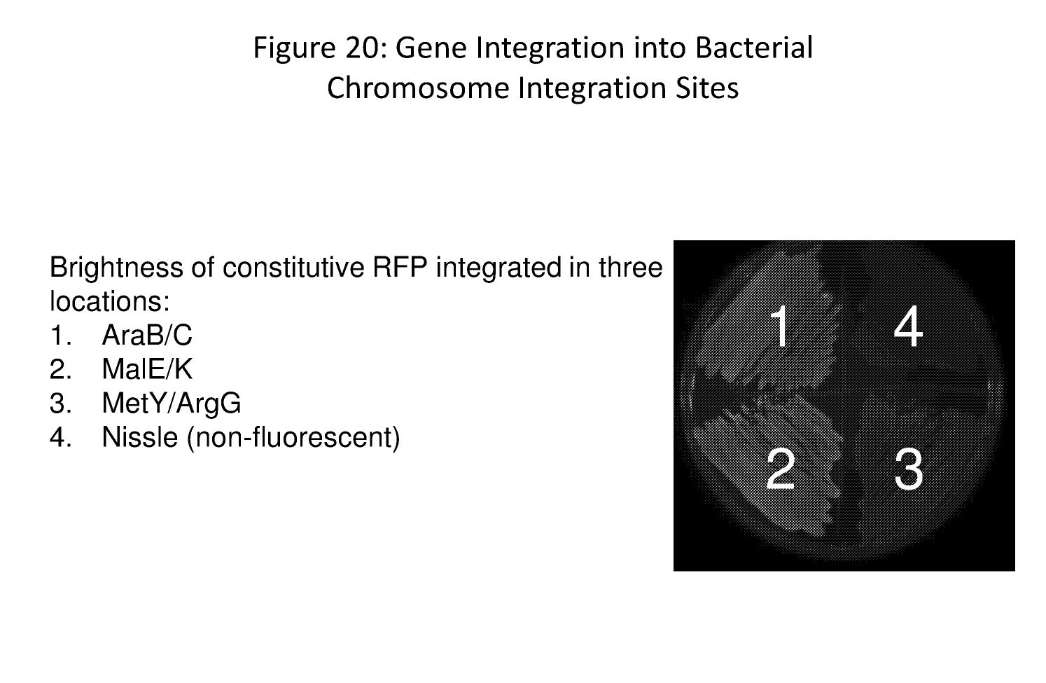

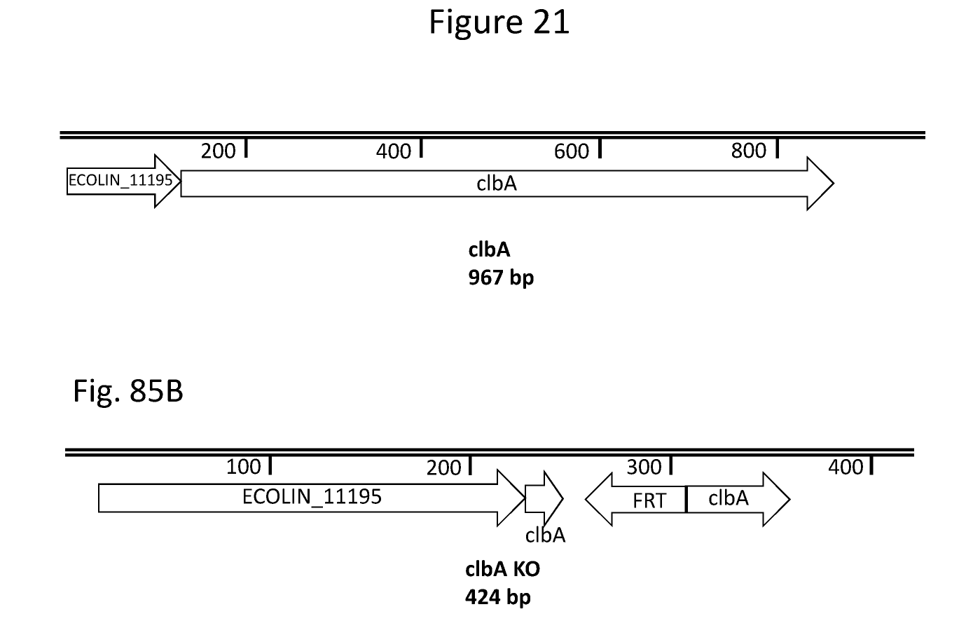

For example, FIG. 19 depicts map of integration sites within the E. coli Nissle chromosome. FIG. 20 depicts three bacterial strains wherein the RFP gene has been successfully integrated into the bacterial chromosome at an integration site.

In Vivo Models

The recombinant bacteria may be evaluated in vivo, e.g., in an animal model. Any suitable animal model of a disease or condition associated with amino acid metabolism, such as cancer, may be used.

Secretion

In some embodiments, the genetically engineered bacteria further comprise a native secretion mechanism or non-native secretion mechanism that is capable of secreting a molecule from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes. In some embodiments, the genetically engineered bacteria further comprise a non-native double membrane-spanning secretion system. Membrane-spanning secretion systems include, but are not limited to, the type I secretion system (T1SS), the type II secretion system (T2SS), the type III secretion system (T3SS), the type IV secretion system (T4SS), the type VI secretion system (T6SS), and the resistance-nodulation-division (RND) family of multi-drug efflux pumps (Pugsley 1993; Gerlach et al., 2007; Collinson et al., 2015; Costa et al., 2015; Reeves et al., 2015; WO2014138324A1, incorporated herein by reference). Mycobacteria, which have a Gram-negative-like cell envelope, may also encode a type VII secretion system (T7SS) (Stanley et al., 2003). With the exception of the T2SS, double membrane-spanning secretions generally transport substrates from the bacterial cytoplasm directly into the extracellular space or into the target cell. In contrast, the T2SS and secretion systems that span only the outer membrane may use a two-step mechanism, wherein substrates are first translocated to the periplasm by inner membrane-spanning transporters, and then transferred to the outer membrane or secreted into the extracellular space. Outer membrane-spanning secretion systems include, but are not limited to, the type V secretion or autotransporter system or autosecreter system (TSSS), the curli secretion system, and the chaperone-usher pathway for pili assembly (Saier, 2006; Costa et al., 2015).

In some embodiments, the genetically engineered bacteria of the invention further comprise a type III or a type III-like secretion system (T3SS) from Shigella, Salmonella, E. coli, Bivrio, Burkholderia, Yersinia, Chlamydia, or Pseudomonas. The T3SS is capable of transporting a protein from the bacterial cytoplasm to the host cytoplasm through a needle complex. The T3SS may be modified to secrete the molecule from the bacterial cytoplasm, but not inject the molecule into the host cytoplasm. Thus, the molecule is secreted into the gut lumen or other extracellular space. In some embodiments, the genetically engineered bacteria comprise said modified T3SS and are capable of secreting the molecule of interest from the bacterial cytoplasm. In some embodiments, the secreted molecule, such as a heterologous protein or peptide comprises a type III secretion sequence that allows the molecule of interest to be secreted from the bacteria.

In some embodiments, a flagellar type III secretion pathway is used to secrete the molecule of interest. In some embodiments, an incomplete flagellum is used to secrete a therapeutic peptide of interest by recombinantly fusing the peptide to an N-terminal flagellar secretion signal of a native flagellar component. In this manner, the intracellularly expressed chimeric peptide can be mobilized across the inner and outer membranes into the surrounding host environment. For example, a modified flagellar type III secretion apparatus in which untranslated DNA fragment upstream of the gene fliC (encoding flagellin), e.g., a 173-bp region, is fused to the gene encoding the polypeptide of interest can be used to secrete heterologous polypeptides (See, e.g., Majander et al., Extracellular secretion of polypeptides using a modified Escherichia coli flagellar secretion apparatus. Nat Biotechnol. 2005 April; 23(4):475-81). In some cases, the untranslated region from the fliC loci, may not be sufficient to mediate translocation of the passenger peptide through the flagella. Here it may be necessary to extend the N-terminal signal into the amino acid coding sequence of FliC, for example using the 173 bp of untranslated region along with the first 20 amino acids of FliC (see, e.g., Duan et al., Secretion of Insulinotropic Proteins by Commensal Bacteria: Rewiring the Gut To Treat Diabetes, Appl. Environ. Microbiol. December 2008 vol. 74 no. 23 7437-7438).

In some embodiments, a Type V Autotransporter Secretion System is used to secrete the molecule of interest, e.g., therapeutic peptide. Due to the simplicity of the machinery and capacity to handle relatively large protein fluxes, the Type V secretion system is attractive for the extracellular production of recombinant proteinsA therapeutic peptide (star) can be fused to an N-terminal secretion signal, a linker, and the beta-domain of an autotransporter. The N-terminal, Sec-dependent signal sequence directs the protein to the SecA-YEG machinery which moves the protein across the inner membrane into the periplasm, followed by subsequent cleavage of the signal sequence. The Beta-domain is recruited to the Bam complex (‘Beta-barrel assembly machinery’) where the beta-domain is folded and inserted into the outer membrane as a beta-barrel structure. The therapeutic peptide is threaded through the hollow pore of the beta-barrel structure ahead of the linker sequence. Once exposed to the extracellular environment, the therapeutic peptide can be freed from the linker system by an autocatalytic cleavage (left side of Bam complex) or by targeting of a membrane-associated peptidase (black scissors; right side of Bam complex) to a complimentary protease cut site in the linker. Thus, in some embodiments, the secreted molecule, such as a heterologous protein or peptide comprises an N-terminal secretion signal, a linker, and beta-domain of an autotransporter so as to allow the molecule to be secreted from the bacteria.

In some embodiments, a Hemolysin-based Secretion System is used to secrete the molecule of interest, e.g., therapeutic peptide. Type I Secretion systems offer the advantage of translocating their passenger peptide directly from the cytoplasm to the extracellular space, obviating the two-step process of other secretion types. The alpha-hemolysin (HlyA) of uropathogenic Escherichia coli. This pathway uses HlyB, an ATP-binding cassette transporter; HlyD, a membrane fusion protein; and TolC, an outer membrane protein. The assembly of these three proteins forms a channel through both the inner and outer membranes. Natively, this channel is used to secrete HlyA, however, to secrete the therapeutic peptide of the present disclosure, the secretion signal-containing C-terminal portion of HlyA is fused to the C-terminal portion of a therapeutic peptide (star) to mediate secretion of this peptide.

In alternate embodiments, the genetically engineered bacteria further comprise a non-native single membrane-spanning secretion system. Single membrane-spanning transporters may act as a component of a secretion system, or may export substrates independently. Such transporters include, but are not limited to, ATP-binding cassette translocases, flagellum/virulence-related translocases, conjugation-related translocases, the general secretory system (e.g., the SecYEG complex in E. coli), the accessory secretory system in mycobacteria and several types of Gram-positive bacteria (e.g., Bacillus anthracis, Lactobacillus johnsonii, Corynebacterium glutamicum, Streptococcus gordonii, Staphylococcus aureus), and the twin-arginine translocation (TAT) system (Saier, 2006; Rigel and Braunstein, 2008; Albiniak et al., 2013). It is known that the general secretory and TAT systems can both export substrates with cleavable N-terminal signal peptides into the periplasm, and have been explored in the context of biopharmaceutical production. The TAT system may offer particular advantages, however, in that it is able to transport folded substrates, thus eliminating the potential for premature or incorrect folding. In certain embodiments, the genetically engineered bacteria comprise a TAT or a TAT-like system and are capable of secreting the molecule of interest from the bacterial cytoplasm. One of ordinary skill in the art would appreciate that the secretion systems disclosed herein may be modified to act in different species, strains, and subtypes of bacteria, and/or adapted to deliver different payloads.

In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

One way to secrete properly folded proteins in gram-negative bacteria—particularly those requiring disulphide bonds—is to target the reducing-environment periplasm in conjunction with a destabilizing outer membrane. In this manner, the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the genetically engineered bacteria have a “leaky” or de-stabilized outer membrane. Destabilizing the bacterial outer membrane to induce leakiness can be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp is the most abundant polypeptide in the bacterial cell existing at ˜500,000 copies per cell and functions as the primary ‘staple’ of the bacterial cell wall to the peptidoglycan. 1. Silhavy, T.J., Kahne, D. & Walker, S. The bacterial cell envelope. Cold Spring Harb Perspect Biol 2, a000414 (2010). TolA-PAL and OmpA complexes function similarly to Lpp and are other deletion targets to generate a leaky phenotype. Additionally, leaky phenotypes have been observed when periplasmic proteases are inactivated. The periplasm is very densely packed with protein and therefore encode several periplasmic proteins to facilitate protein turnover. Removal of periplasmic proteases such as degS, degP or nlpl can induce leaky phenotypes by promoting an excessive build-up of periplasmic protein. Mutation of the proteases can also preserve the effector polypeptide by preventing targeted degradation by these proteases. Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated lpp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes, in some embodiments, the engineered bacteria have one or more deleted or mutated periplasmic protease genes. In some embodiments, the engineered bacteria have one or more deleted or mutated periplasmic protease genes selected from degS, degP, and nlpl. In some embodiments, the engineered bacteria have one or more deleted or mutated gene(s), selected from lpp, ompA, ompF, tolA, tolB, pal, degS, degP, and nlpl genes.

To minimize disturbances to cell viability, the leaky phenotype can be made inducible by placing one or more membrane or periplasmic protease genes, e.g., selected from lpp, ompA, ompF, tolA, tolB, pal, degS, degP, and nlpl, under the control of an inducible promoter. For example, expression of lpp or other cell wall stability protein or periplasmic protease can be repressed in conditions where the therapeutic polypeptide needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., over expression of colicins or the third topological domain of TolA, which peptide overexpression can be induced in conditions in which the therapeutic polypeptide needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

Table 11 and Table 12A list secretion systems for Gram positive bacteria and Gram negative bacteria. These can be used to secrete polypeptides, proteins of interest or therapeutic protein(s) from the engineered bacteria, which are reviewed in Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety.

TABLE 11
(a) Secretion systems for gram positive
bacteria

| Bacterial Strain | Relevant Secretion System |
|---|---|
| C. novyi-NT (Gram+) | Sec pathway |
| | Twin-arginine (TAT) pathway |
| C. butryicum (Gram+) | Sec pathway |
| | Twin-arginine (TAT) pathway |
| Listeria monocytogenes (Gram +) | Sec pathway |
| | Twin-arginine (TAT) pathway |

TABLE 12A
(b) Secretion Systems for Gram negative bacteria
Protein secretary pathways (SP) in gram-negative bacteria and their descendants

| | | | | | | | |
|---|---|---|---|---|---|---|---|
| Type | | | Bac- | | | # Protein/ | Energy |
| (Abbreviation) | Name | TC#² | teria | Archaea | Eukarya | System | Source |

IMPS-Gram-negative bacterial inner membrane
channel-forming translocases

| | | | | | | | |
|---|---|---|---|---|---|---|---|
| ABC | ATP binding | 3.A.1 | + | + | + | 3-4 | ATP |
| (SIP) | cassette | | | | | | |
| | translocase | | | | | | |
| SEC | General | 3.A.5 | + | + | + | ~12 | GTP |
| (IISP) | secretory | | | | | | OR |
| | translocase | | | | | | ATP + |
| | | | | | | | PMF |
| Fla/Path | Flagellum/ | 3.A.6 | + | − | − | >10 | ATP |
| (IIISP) | virulence- | | | | | | |
| | related | | | | | | |
| | translocase | | | | | | |
| Conj | Conjugation- | 3.A.7 | + | − | − | >10 | ATP |
| (IVSP) | related | | | | | | |
| | translocase | | | | | | |
| Tat (IISP) | Twin- | 2.A.64 | + | + | + | 2-4 | PMF |
| | arginine | | | | (chloro- | | |
| | targeting | | | | plasts) | | |
| | translocase | | | | | | |
| Oxa1 | Cytochrome | 2.A.9 | + | + | + | 1 | None |
| (YidC) | oxidase | | | | (mitochondria | | or |
| | biogenesis | | | | chloroplasts) | | PMF |
| | family | | | | | | |
| MscL | Large | 1.A.22 | + | + | + | 1 | None |
| | conductance | | | | | | |
| | mechano- | | | | | | |
| | sensitive channel | | | | | | |
| | family | | | | | | |
| Holins | Holin | 1.E.1 • | + | − | − | 1 | None |
| | functional | 21 | | | | | |
| | superfamily | | | | | | |

Eukaryotic Organelles

| | | | | | | | |
|---|---|---|---|---|---|---|---|
| MPT | Mitochondrial | 3.A.B | − | − | + | >20 | ATP |
| | protein | | | | (mito- | | |
| | translocase | | | | chondrial) | | |
| CEPT | Chloroplast | 3.A.9 | (+) | − | + | ≥3 | GTP |
| | envelope | | | | (chloroplasts) | | |
| | protein | | | | | | |
| | translocase | | | | | | |
| Bcl-2 | Eukaryotic | 1.A.21 | − | − | + | 1? | None |
| | Bcl-2 family | | | | | | |
| | (programmed | | | | | | |
| | cell death) | | | | | | |

Gram-negative bacterial outer membrane channel-forming translocases

| | | | | | | | |
|---|---|---|---|---|---|---|---|
| MTB | Main | 3.A.15 | +^{b} | − | − | ~14 | ATP; |
| (IISP) | terminal | | | | | | PMF |
| | branch of the | | | | | | |
| | general | | | | | | |
| | secretory | | | | | | |
| | translocase | | | | | | |
| FUP AT-1 | Fimbrial | 1.B.11 | +^{b} | − | − | 1 | None |
| | usher protein | | | | | | |
| | Autotrans- | 1.B.12 | +^{b} | | − | 1 | None |
| | porter-1 | | | | | | |
| AT-2 | Autotrans- | 1.B.40 | +^{b} | − | − | 1 | None |
| OMF | porter-2 | 1.B.17 | +^{b} | | +(?) | 1 | None |
| (ISP) | | | | | | | |
| TPS | | 1.B.20 | + | − | + | 1 | None |
| Secretin | | 1.B.22 | +^{b} | | − | 1 | None |
| (IISP and | | | | | | | |
| IISP) | | | | | | | |
| OmpIP | Outer | 1.B.33 | + | - | + | ≥4 | None |
| | membrane | | | | (mito- | | ? |
| | insertion | | | | chondria; | | |
| | porin | | | | chloroplasts) | | |

The above tables for gram positive and gram negative bacteria list secretion systems that can be used to secrete polypeptides and other molecules from the engineered bacteria, which are reviewed in Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety.

TABLE 12B.
(c) Comparison of Secretion systems for
secretion of polypeptide from engineered
bacteria

| Secretion | | | | |
|---|---|---|---|---|
| System | Tag | Cleavage | Advantages | Other features |
| Modified | mRNA | No | No peptide tag | May not be as |
| Type III | (or N- | cleavage | Endogenous | suited for larger |
| (flagellar) | terminal) | necessary | | proteins Deletion |
| | | | | of flagellar |
| | | | | genes |
| Type V | N- and | Yes | Large proteins | 2-step secretion |
| autotransport | C- | | Endogenous | |
| | terminal | | Cleavable | |
| Type I | C- | No | | Tag; Exogenous |
| | terminal | | | Machinery |
| Diffusible | N- | Yes | Disulfide bond | May affect cell |
| Outer | terminal | | formation | fragility/ |
| Membrane | | | | survivability/ |
| (DOM) | | | | growth/yield |

In some embodiments, one or more amino acid catabolic enzymes described herein are secreted. In some embodiments, the one or more amino acid catabolic enzymes described herein are further modified to improve secretion efficiency, decreased susceptibility to proteases, stability, and/or half-life.

Pharmaceutical Compositions and Formulations

Pharmaceutical compositions comprising the genetically engineered bacteria described herein may be used to treat, manage, ameliorate, and/or prevent cancer or symptom(s) associated with cancer. Pharmaceutical compositions comprising one or more genetically engineered bacteria, alone or in combination with prophylactic agents, therapeutic agents, and/or pharmaceutically acceptable carriers are provided.

Pharmaceutical compositions comprising the genetically engineered microorganisms of the invention may be used to treat, manage, ameliorate, and/or prevent a disorder associated with amino acid catabolism or symptom(s) associated with diseases or disorders associated with amino acid catabolism. Pharmaceutical compositions of the invention comprising one or more genetically engineered bacteria, and/or one or more genetically engineered virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., to express an amino acid catabolism enzyme. In alternate embodiments, the pharmaceutical composition comprises two or more species, strains, and/or subtypes of bacteria that are each engineered to comprise the genetic modifications described herein, e.g., to express an amino acid catabolism enzyme.

The pharmaceutical compositions of the invention described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 104 to 1012 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria or genetically engineered virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microroganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as par of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Methods of Treatment

Further disclosed herein are methods of treating diseases associated with amino acid metabolism. In some embodiments, disclosed herein are methods for reducing, ameliorating, or eliminating one or more symptom(s) associated with these diseases or disorders.

As used herein the terms “disease associated with amino acid metabolism” or a “disorder associated with amino acid metabolism” is a disease or disorder involving the abnormal, e.g., increased, levels of one or more amino acids in a subject. In one embodiment, a disease or disorder associated with amino acid metabolism is a cancer. In another embodiment, a disease or disorder associated with amino acid metabolism is a metabolic disease. In one embodiment, the cancer is glioma. In another embodiment, the cancer is breast cancer. In another embodiment, the cancer is melanoma. In another embodiment, the cancer is hepatocarcinoma. In another embodiment, the cancer is acute lymphoblastic leukemia (ALL). In another embodiment, the cancer is ovarian cancer. In another embodiment, the cancer is prostate cancer. In another embodiment, the cancer is lymphoblastic leukemia. In another embodiment, the cancer is non-small cell lung cancer.

In some embodiments, the disclosure provides methods for reducing, ameliorating, or eliminating one or more symptom(s) associated with these diseases.

The method may comprise preparing a pharmaceutical composition with at least one genetically engineered species, strain, or subtype of bacteria described herein, and administering the pharmaceutical composition to a subject in a therapeutically effective amount. In some embodiments, the genetically engineered bacteria disclosed herein are administered orally, e.g., in a liquid suspension. In some embodiments, the genetically engineered bacteria are lyophilized in a gel cap and administered orally. In some embodiments, the genetically engineered bacteria are administered via a feeding tube or gastric shunt. In some embodiments, the genetically engineered bacteria are administered rectally, e.g., by enema. In some embodiments, the genetically engineered bacteria are administered topically, intraintestinally, intrajejunally, intraduodenally, intraileally, and/or intracolically. In one embodiment, the genetically engineered bacteria are injected directly into a tumor.

In certain embodiments, administering the pharmaceutical composition to the subject reduces the level of an amino acid in a subject. In some embodiments, the methods of the present disclosure may reduce the level of an amino acid in a subject by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing the amino acid concentration in a subject before and after administration of the pharmaceutical composition. In some embodiments, the method of treating or ameliorating a disease or disorder allows one or more symptoms of the condition or disorder to improve by at least about 10%, 20%, 30%, 40%, 50%, 60%, 70%, 80%, 90%, 95%, or more Amino acid levels may be measured by methods known in the art (see amino acid catabolism enzyme section, supra).

Before, during, and after the administration of the pharmaceutical composition, ammonia concentrations in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce amino acid concentrations in a subject to undetectable levels, or to less than about 1%, 2%, 5%, 10%, 20%, 25%, 30%, 40%, 50%, 60%, 70%, 75%, or 80% of the subject's amino acid concentration(s) prior to treatment.

The methods disclosed herein may further comprise isolating a sample from the subject prior to administration of a composition and determining the level of the amino acid(s) in the sample. In some embodiments, the methods may further comprise isolating a sample from the subject after to administration of a composition and determining the level of amino acid(s) in the sample.

In certain embodiments, the genetically engineered bacteria comprising an amino acid catabolism enzyme is E. coli Nissle. The genetically engineered bacteria may be destroyed, e.g., by defense factors in the gut or blood serum (Sonnenborn et al., 2009), or by activation of a kill switch, several hours or days after administration. Thus, the pharmaceutical composition comprising the mutant arginine regulon may be re-administered at a therapeutically effective dose and frequency. Length of Nissle residence in vivo in mice can be determined. In alternate embodiments, the genetically engineered bacteria are not destroyed within hours or days after administration and may propagate and colonize the gut.

The methods disclosed herein may comprise administration of a composition alone or in combination with one or more additional therapies, e.g., chemotherapy. The pharmaceutical composition may be administered alone or in combination with one or more additional therapeutic agents, including but not limited to, sodium phenylbutyrate, sodium benzoate, and glycerol phenylbutyrate. The methods may also comprise following an amino acid restricted diet.

An important consideration in the selection of the one or more additional therapeutic agents is that the agent(s) should be compatible with the genetically engineered bacteria disclosed herein, e.g., the agent(s) must not kill the bacteria. In some embodiments, the pharmaceutical composition is administered with food. In alternate embodiments, the pharmaceutical composition is administered before or after eating food. The pharmaceutical composition may be administered in combination with one or more dietary modifications, e.g., low-protein diet or amino acid supplementation. The dosage of the pharmaceutical composition and the frequency of administration may be selected based on the severity of the symptoms and the progression of the disorder. The appropriate therapeutically effective dose and/or frequency of administration can be selected by a treating clinician.

### EXAMPLES

The present disclosure is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

Development of Recombinant Bacterial Cells

### Example 1. Construction of Plasmids Encoding Amino Acid Catabolism Enzymes

An amino acid catabolism gene is synthesized (Genewiz), fused to the Tet promoter, cloned into the high-copy plasmid pUC57-Kan by Gibson assembly, and transformed into E. coli DH5a as described herein to generate the plasmid pTet-AAC.

### Example 2. Generation of Recombinant Bacteria Comprising an Amino Acid Catabolism Enzyme

The pTet-AAC plasmid described above is transformed into E. coli Nissle, DH5a, or PIR1. All tubes, solutions, and cuvettes are pre-chilled to 4° C. An overnight culture of E. coli (Nissle, DH5a or PIR1) is diluted 1:100 in 4 mL of LB and grown until it reaches an OD₆₀₀ of 0.4-0.6. 1 mL of the culture is then centrifuged at 13,000 rpm for 1 min in a 1.5 mL microcentrifuge tube and the supernatant is removed. The cells are then washed three times in pre-chilled 10% glycerol and resuspended in 40 uL pre-chilled 10% glycerol. The electroporator is set to 1.8 kV. 1 uL of a pTet-AAC miniprep is added to the cells, mixed by pipetting, and pipetted into a sterile, chilled 1 mm cuvette. The dry cuvette is placed into the sample chamber, and the electric pulse is applied. 500 uL of room-temperature SOC media is immediately added, and the mixture is transferred to a culture tube and incubated at 37° C. for 1 hr. The cells are spread out on an LB plate containing 50 ug/mL Kanamycin for pTet-AAC.

Functional Assays Using Recombinant Bacterial Cells

### Example 3. Functional Assay Demonstrating that the Recombinant Bacterial Cells Decrease Amino Acid Concentration

For in vitro studies, all incubations will be performed at 37° C. Cultures of E. coli Nissle containing pTet-AAC are grown overnight in LB and then diluted 1:100 in LB. The cells are grown with shaking (250 rpm) to early log phase with the appropriate antibiotics. Anhydrous tetracycline (ATC) is added to cultures at a concentration of 100 ng/mL to induce expression of the amino acid catabolism enzyme, and bacteria are grown for another 3 hours. Culture broths are then inoculated at 20% in flasks containing fresh LB culture media containing excess amino acids and grown for 16 hours with shaking (250 rpm).

A “medium blank” for each culture condition broth is also prepared whereby the “medium blank” is not inoculated with bacteria but treated under the same conditions as the inoculated broths. Following the 16 hour incubation period, broth cultures are pasteurized at 90° C. for 15 minutes, centrifuged at 5,000 rpm for 10 minutes, and supernatants filtered with a 0.45 micron filter.

Amino acid levels and activity in the supernatants is determined. Briefly, amino acid concentrations can be assessed using the assays described above in each amino acid catabolism enzyme subsection.

### Example 4. In Vivo Studies Demonstrating that the Recombinant Bacterial Cells Decrease Amino Acid Concentration

For in vivo studies, a mouse model known in the artis used. The mice can be inoculated with recombinant bacteria comprising an amino acid catabolism enzyme (as described herein) or control bacteria. Body weight, plasma samples, and fecal samples can be taken throughout the duration of the study. Upon conclusion of the study, the mice can be killed, and internal organs (liver, spleen, intestines) and fat pads can be removed and assayed. Treatment efficacy is determined, for example, by measuring tumor size and levels of amino acids. A decrease in tumor size or levels of amino acids after treatment with the recombinant bacterial cells indicates that the recombinant bacterial cells described herein are effective for treating disorders in which amino acids are detrimental, such as cancer.

Additionally, throughout the study, phenotypes of the mice can also be analyzed. A decrease in the number of symptoms associated with disorders in which amino acids are detrimental, for example, cancer, further indicates the efficacy of the recombinant bacterial cells described herein for treating disorders associated with amino acid metabolism, such as cancer.

### Example 5. Construction of Plasmids Encoding Branched Chain Amino Acid Importers and Branched Chain Amino Acid Catabolism Enzyme

The kivD gene of Lactococcus lactis IFPL730 was synthesized (Genewiz), fused to the Tet promoter, cloned into the high-copy plasmid pUC57-Kan by Gibson assembly and transformed into E. coli DH5a to generate the plasmid pTet-kivD. The bkd operon of Pseudomonas aeruginosa PAO1 fused to the Tet promoter was synthesized (Genewiz) and cloned into the high-copy plasmid pUC57-Kan to generate the plasmid pTet-bkd. The bkd operon of Pseudomonas aeruginosa PAO1 fused to the leuDH gene from PA01 and the Tet promoter was synthesized (Genewiz) and cloned into the high-copy plasmid pUC57-Kan to generate the plasmid pTet-leuDH-bkd. The livKHMGF operon from E. coli Nissle fused to the Tet promoter was synthesized (Genewiz), cloned into the pKIKO-lacZ plasmid by Gibson assembly and transformed into E. coli PIR1 to generate the pTet-livKHMGF.

### Example 6. Generation of Recombinant Bacterial Comprising a Genetic Modification that Reduces Export of a Branched Chain Amino Acid

E. coli Nissle was transformed with the pKD46 plasmid encoding the lambda red proteins under the control of an arabinose-inducible promoter as follows. An overnight culture of E. coli Nissle grown at 37° C. was diluted 1:100 in 4 mL of lysogeny broth (LB) and grown at 37° C. until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pKD46 miniprep was added to the cells, mixed by pipetting, and pipetted into a sterile, chilled 1 mm cuvette. The cuvette was placed into the sample chamber, and the electric pulse was applied. 500 uL of room-temperature SOC media was immediately added, and the mixture was transferred to a culture tube and incubated at 30° C. for 1 hr. The cells were spread out on an LB plate containing 100 ug/mL carbenicillin and incubated at 30° C.

A ΔleuE deletion construct with 77 bp and a 100 bp flanking leuE homology regions and a kanamycin resistant cassette flanked by FRT recombination site (SEQ ID NO: 6) was generated by PCR, column-purified and transformed into E. coli Nissle pKD46 as follows. An overnight culture of E. coli Nissle pKD46 grown in 100 ug/mL carbenicillin at 30° C. was diluted 1:100 in 5 mL of LB supplemented with 100 ug/mL carbenicillin, 0.15% arabinose and grown until it reaches an OD₆₀₀ of 0.4-0.6. The bacteria were aliquoted equally in five 1.5 mL microcentrifuge tubes, centrifuged at 13,000 rpm for 1 min and the supernatant was removed. The cells were then washed three times in pre-chilled 10% glycerol and combined in 50 uL pre-chilled 10% glycerol. The electroporator was set to 1.8 kV. 2 uL of a the purified ΔleuE deletion PCR fragment are then added to the cells, mixed by pipetting, and pipetted into a sterile, chilled 1 mm cuvette. The cuvette was placed into the sample chamber, and the electric pulse was applied. 500 uL of room-temperature SOC media was immediately added, and the mixture was transferred to a culture tube and incubated at 37° C. for 1 hr. The cells were spread out on an LB plate containing 50 ug/mL kanamycin. Five kanamycin-resistant transformants were then checked by colony PCR for the deletion of the leuE locus.

The kanamycin cassette was then excised from the ΔleuE deletion strain as follows. ΔleuE was transformed with the pCP20 plasmid encoding the Flp recombinase gene. An overnight culture of ΔleuE grown at 37° C. in LB with 50 ug/mL kanamycin was diluted 1:100 in 4 mL of LB and grown at 37° C. until it reaches an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pCP20 miniprep was added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 30° C. for 1 hr. The cells were spread out on an LB plate containing 100 ug/mL carbenicillin and incubated at 30° C. Eight transformants were then streaked on an LB plate and were incubated overnight at 43° C. One colony per transformant was picked and resuspended in 10 uL LB and 3 uL of the suspension were pipetted on LB, LB with 50 ug/mL Kanamycin or LB with 100 ug/mL carbenicillin. The LB and LB Kanamycin plates were incubated at 37° C. and the LB Carbenicillin plate was incubated at 30° C. Colonies showing growth on LB alone were selected and checked by PCR for the excision of the Kanamycin cassette.

### Example 7. Generation of Recombinant Bacteria Comprising a Transporter of a Branched Chain Amino Acid and/or a Branched Chain Amino Acid Catabolism Enzyme and Lacking an Exporter of a Branched Chain Amino Acid

pTet-kivD, pTet-bkd, pTet-leuDH-bkd and pTet-livKHFGF plasmids described above were transformed into E. coli Nissle (pTet-kivD), Nissle (pTet-kivD, pTet-bkd, pTet-leuDH-bkd), DH5α (pTet-kivD, pTet-bkd, pTet-leuDH-bkd) or PIR1 (pTet-livKHMGF). All tubes, solutions, and cuvettes were pre-chilled to 4° C. An overnight culture of E. coli (Nissle, ΔleuE, DH5α or PIR1) was diluted 1:100 in 4 mL of LB and grown until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pTet-kivD, pTet-bkd, pTet-leuDH-bkd or pTet-livKHMGF miniprep was added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 37° C. for 1 hr. The cells were spread out on an LB plate containing 50 ug/mL Kanamycin for pTet-kivD, pTet-bkd and pTet-leuDH-bkd or 100 ug/mL carbenicillin for pTet-livKHMGF.

### Example 8. Generation of Recombinant Bacteria Comprising a Transporter of a Branched Chain Amino Acid and a Genetic Modification that Reduces Export of a Branched Chain Amino Acid

E. coli Nissle ΔleuE was transformed with the pKD46 plasmid encoding the lambda red proteins under the control of an arabinose-inducible promoter as follows. An overnight culture of E. coli Nissle ΔleuE grown at 37° C. was diluted 1:100 in 4 mL of LB and grown at 37° C. until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pKD46 miniprep was added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 30° C. for 1 hr. The cells were spread out on an LB plate containing 100 ug/mL carbenicillin and incubated at 30° C.

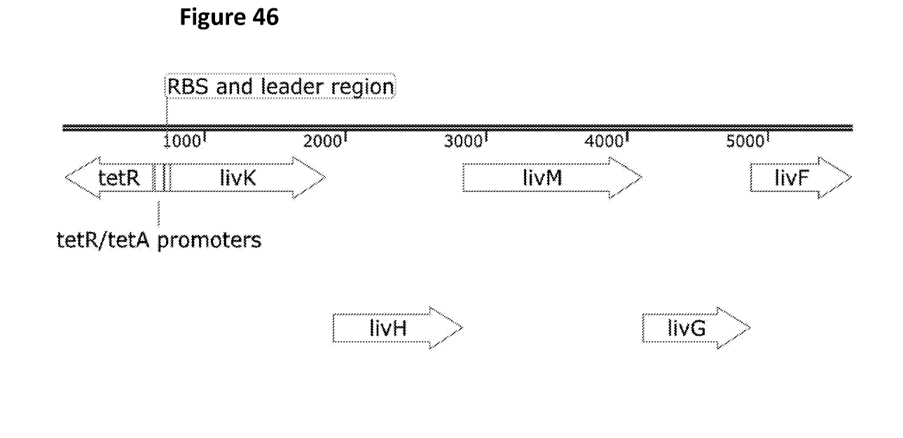

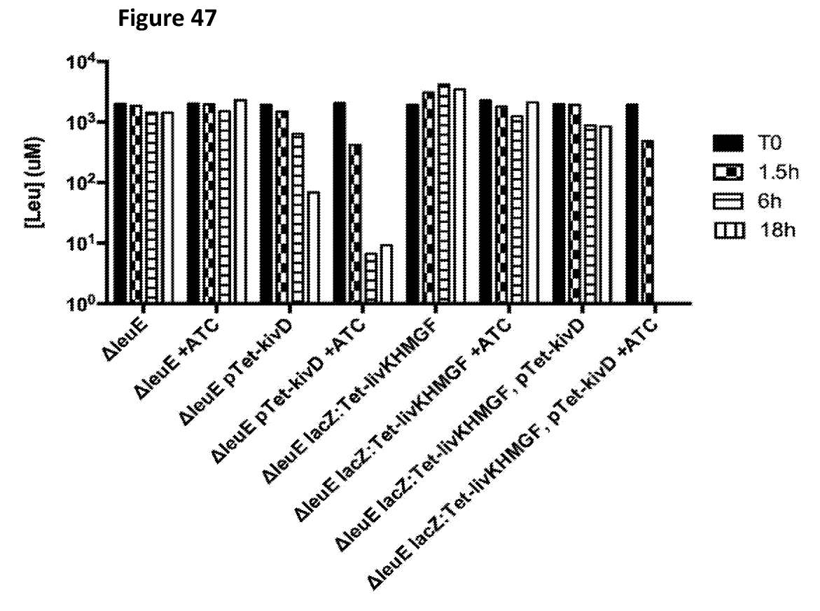

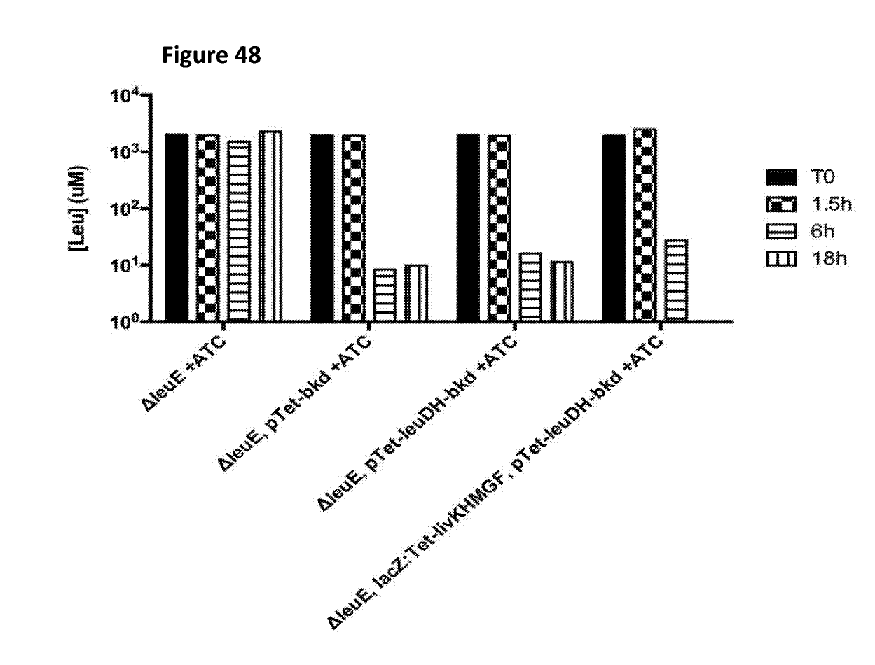

The DNA fragment used to integrate Tet-livKHMGF into E. coli Nissle lacZ (FIG. 46) was amplified by PCR from the pTet-livKHMGF plasmid, column-purified and transformed into ΔleuE pKD46 as follows. An overnight culture of the E. coli Nissle ΔleuE pKD46 strain grown in LB at 30° C. with 100 ug/mL carbenicillin was diluted 1:100 in 5 mL of lysogeny broth (LB) supplemented with 100 ug/mL carbenicillin, 0.15% arabinose and grown at 30° C. until it reached an OD₆₀₀ of 0.4-0.6. The bacteria were aliquoted equally in five 1.5 mL microcentrifuge tubes, centrifuged at 13,000 rpm for 1 min and the supernatant was removed. The cells were then washed three times in pre-chilled 10% glycerol and combined in 50 uL pre-chilled 10% glycerol. The electroporator was set to 1.8 kV. 2 uL of a the purified Tet-livKHMGF PCR fragment were then added to the cells, mixed by pipetting, and pipetted into a sterile, chilled 1 mm cuvette. The dry cuvette was placed into the sample chamber, and the electric pulse was applied. 500 uL of room-temperature SOC media was immediately added, and the mixture was transferred to a culture tube and incubated at 37° C. for 1 hr. The cells were spread out on an LB plate containing 20 ug/mL chloramphenicol, 40 ug/mL X-Gal and incubated overnight at 37° C. White chloramphenicol resistant transformants were then checked by colony PCR for integration of Tet-livKHMGF into the lacZ locus.

Functional assays using recombinant bacterial cells

### Example 9. Functional Assay Demonstrating that the Recombinant Bacterial Cells Disclosed Herein Decrease Branched Chain Amino Acid Concentration

For in vitro studies, all incubations were performed at 37° C. Cultures of E. coli Nissle ΔleuE, ΔleuE+pTet-kivD, ΔleuE+pTet-bkd, ΔleuE+pTet-leuDH-bkd, ΔleuE lacZ:Tet-livKHMGF, ΔleuE lacZ:Tet-livKHMGF+pTet-kivD, ΔleuE lacZ:Tet-livKHMGF+pTet-bkd, ΔleuE lacZ:Tet-livKHMGF+pTet-leuDH-bkd were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD, Bkd, LeuDH and LivKHFMG, and bacteria were grown for another 3 hours. Bacteria were then pelleted, washed, and resuspended in minimal media, and supplemented with 0.5% glucose and 2 mM leucine. Aliquots were removed at 0 h, 1.5 h, 6 h and 18 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in 90 uL 10% acetonitrile, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 95% A, 5% B

0.5 min: 95% A, 5% B

1 min: 10% A, 90% B

2.5 min: 10% A, 90% B

2.51 min: 95% A, 5% B

3.5 min: 95% A, 5% B

The Q1/Q3 transitions used for leucine and L-leucine-5,5,5-d₃ were 132.1/86.2 and 135.1/89.3 respectively.

Leucine was rapidly degraded by the expression of kivD in the Nissle ΔleuE strain. After 6 h of incubation, leucine concentration droped by over 99% in the presence of ATC. This effect was even more pronounced in the case of ΔleuE expressing both kivD and the leucine transporter livKHMGF where leucine is undetectable after 6 h of incubation. The expression of the bkd complex also leads rapidly to the degradation of leucine. After 6 h of incubation, 99% of leucine was degraded. The expression of the leucine transporter livKHMGF, in parallel with the expression of leuDH and bkd leads to the complete degradation of leucine after 18 h.

### Example 10. Simultaneous Degradation of Branched Chain Amino Acids by Recombinant Bacteria Expressing a Branched Chain Amino Acid Catabolism Enzyme and an Importer of a Branched Chain Amino Acid

In these studies, all incubations were performed at 37° C. Cultures of E. coli Nissle, Nissle+pTet-kivD, ΔleuE+pTet-kivD, ΔleuE lacZ:Tet-livKHMGF+pTet-kivD were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD and LivKHFMG, and bacteria were grown for another 3 hours. Bacteria were then pelleted, washed, and resuspended in minimal media, and supplemented with 0.5% glucose and the three branched chain amino acids (leucine, isoleucine and valine, 2 mM each). Aliquots were removed at Oh, 1.5 h, 6 h and 18 h for leucine, isoleucine and valine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

Isoleucine: 132.1/86.2

Valine: 118.1/72

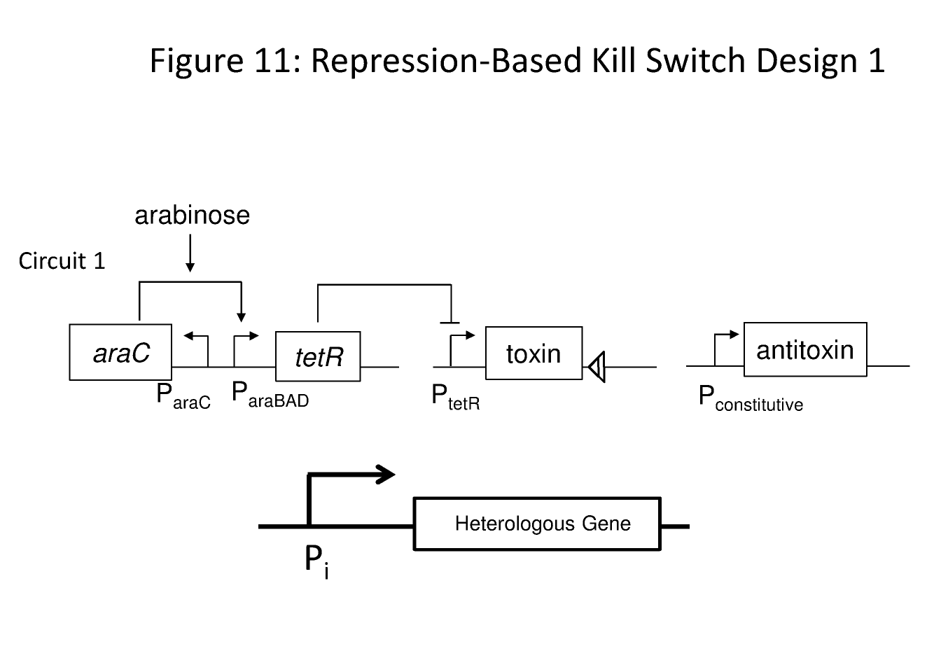

As shown in FIGS. 11A-11C, leucine, isoleucine and valine were all degraded by the expression of kivD in E. coli Nissle. At 18 h, 96.8%, 67.2% and 52.1% of leucine, isoleucine and valine respectively were degraded in Nissle expressing kivD in the presence of ATC. The efficiency of leucine and isoleucine degradation was further improved by expressing kivD in the ΔleuE background strain with a 99.8% leucine and 80.6% isoleucine degradation at 18 h. Finally, an additional increase in leucine and isoleucine degradation was achieved by expressing the leucine transporter livKHMGF in the Nissle ΔleuE pTet-kivD strain with a 99.98% leucine and 95.5% isoleucine degradation at 18 h. No significant improvement in valine degradation was observed in the ΔleuE deletion strain expressing livKHMGF.

### Example 11. Degradation of Leucine and its Ketoacid Derivative, Ketoisocaproate (KIC) by Recombinant Bacterial Cells In Vitro

Leucine and its ketoacid derivative, alpha-ketoisocaproate (KIC), are two major metabolites which accumulate in insulin resistant patients. Different synthetic probiotic E. coli Nissle strains were engineered to degrade leucine and KIC in order to determine the rate of degradation of leucine and KIC in these strains.

All strains were derived from the human probiotic strain E. coli Nissle 1917. A ΔleuE deletion strain (deleted for the leucine exporter leuE) was generated by lambda red-recombination. A copy of the high-affinity leucine ABC transporter livKHMGF under the control of a tetracycline-inducible promoter (Ptet) was inserted into the lacZ locus of the ΔleuE deletion strain by lambda-red recombination. In order to avoid endogenous production of BCAA and KIC, the biosynthetic gene ilvC was deleted in the ΔleuE; lacZ:tetR-Pₜₑₜ-livKHMGF strain by P1 transduction using the ΔilvC BW25113 E. coli strain as donor to generate the SYN469 strain (ΔleuE ΔilvC; lacZ:tetR-Pₜₑₜ-livKHMGF).

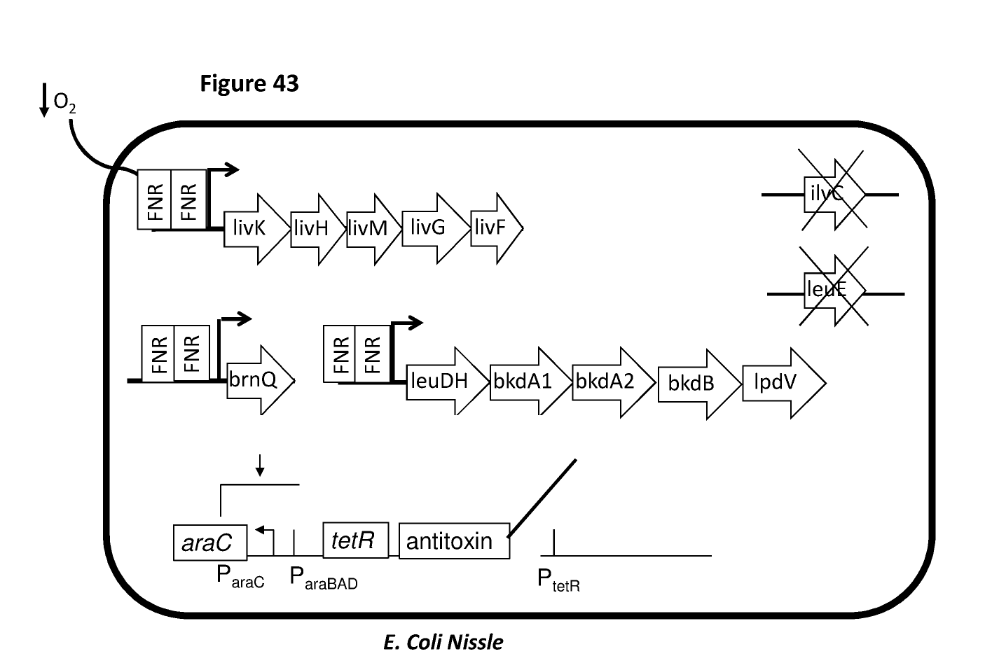

The SYN469 strain was then transformed with five different constructs under the control of Ptet on the high-copy plasmid pUC57-Kan (FIG. 43). The components of the constructs were:

the leucine dehydrogenase leuDH derived from Pseudomonas aeruginosa PAO1, which catalyzes the reversible deamination of branched chain amino acids (i.e., leucine, valine and isoleucine),

the branched chain amino acid aminotransferase ilvE from E. coli Nissle, which catalyzes the reversible deamination of branched chain amino acids (i.e., leucine, valine and isoleucine),

the ketoacid decarboxylase kivD derived from Lactococcus lactis strain IFPL730, which catalyzes the decarboxylation of branched chain amino acids, and/or

the alcohol dehydrogenase adh2 derived from Saccharomyces cerevisiae, which catalyzes the conversion of branched chain amino acid-derived aldehydes to their respective alcohols.

Specifically, the following constructs were generated: Ptet-kivD (SYN479), ptet-kivD-leuDH (SYN467), Ptet-kivD-adh2 (SYN949), ptet-leuDH-kivD-adh2 (SYN954), and Ptet-ilvE-kivD-adh2 (SYN950).

SYN467, SYN469, SYN479, SYN949, SYN950 and SYN954 were grown overnight at 37° C. and 250 rpm in 4 mL of LB supplemented with 100 μg/mL kanamycin for SYN467, SYN479, SYN949, SYN950 and SYN954. Cells were diluted 100 fold in 4 mL LB (with 100 μg/mL kanamycin for SYN467, SYN479, SYN949, SYN950 and SYN954) and grown for 2 h at 37° C. and 250 rpm. Cells were split in two 2 mL culture tubes, and one 2 mL culture tube was induced with 100 ng/mL anhydrotetracycline (ATC) to activate the Ptet promoter. After 1 h induction, the two 2 mL culture tubes were split in four 1 mL microcentrifuge tubes. The cells were spun down at maximum speed for 30 seconds in a microcentrifuge. The supernatant was removed and the pellet re-suspended in 1 mL M9 medium 0.5% glucose. The cells were spun down again at maximum speed for 30 seconds and resuspended in 1 mL M9 medium 0.5% glucose supplemented with 2 mM leucine or 2 mM KIC. Serial dilutions of the different cell suspensions were plated to determine the initial number of CFUs. The cells were transferred to a culture tube and incubated at 37° C. and 250 rpm for 3 h. 150 μL of cells were collected at 0 h, 1 h, 2 h and 3 h after addition of leucine or KIC for quantification by LC-MS/MS. Briefly, 100 uL aliquots were centrifuged at 4,500 rpm for 10 min. 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2 in positive mode

KIC: 129.1/129.1 in negative mode

The rate of degradation (in μmol/10⁹ CFUs/hr) was calculated for leucine and KIC.

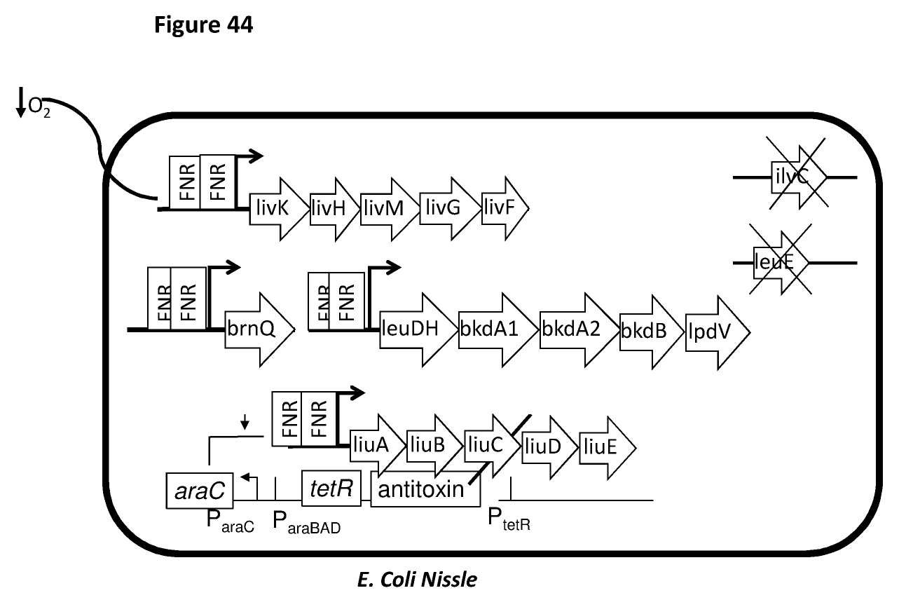

FIG. 44 and FIG. 45 demonstrate that the different recombinant bacteria are able to debrade both leucine and MC. The best performing strain was SYN950, with a 0.8 and 2.2 μmol/10⁹ CFUs/hr degradation rate for leucine and MC, respectively.

The following table summarizes other experimental data generated in the course of evaluating leucine-degrading circuits:

| Feature | Insights Gained |
|---|---|
| Branched chain aa | Intrinsic production of valine by engineered strain |
| recycling (E. coli | does not interfere with leucine degradation |
| can synthesize | |
| and excrete | |
| valine) | |
| Gene expression | High copy expression of kivD enhances degradation |
| level | rates → seeking switches with stronger activation |
| | levels |
| Co-factor | Adding exogenous thiamine does not increase |
| requirement | activity → endogenous pools sufficient |
| Environmental | pH optimum = 6.5 → reaction should work well |
| and assay pH | under GI and physiological conditions |
| Carbon source | Glucose drives optimal reaction rates → no |
| utilization and | evidence for inhibition by glycolysis (e.g., acid) |
| byproducts | byproducts |

Additional measures that may be taken to improve branched chain amino acid degradation rate include:

| Potential | |
|---|---|
| limitation | Test |
| BCAA uptake by | Test additional BCAA transporters |
| cell is rate- | Establish genetic selections for transporter mutants |
| limiting | with increased activity |
| BCAA-derived | Increase ADH2 expression/activity to convert the |
| aldehydes inhibit | aldehydes into their respective alcohol |
| KivD | |
| Slow conversion | Express leuDH on a separate transcript from kivD |
| of BCAAs into | Increase transcription rates for leuDH and kivD |
| their ketoacids | Overexpress the endogenous ilvE (BCAT) |
| | Identify KivD or LeuDH variants with increased |
| | enzymatic activity |
| Slow folding or | Increase cellular osmolytes concentration (NaCl + |
| misfolding of | betaine) Lower induction temperature |
| BCDH or KivD | Induce the expression of endogenous chaperones |
| | (heat-shock, benzyl alcohol) |
| | Express chaperones (dnaK-dnaJ-grpE, groES-groEL) |

### Example 12. Construction of Plasmids Encoding Branched Chain Amino Acid Catabolism Enzymes, Including a BCAA Deaminating Enzyme, an Alpha-Keto-Acid Decarboxylase, an Alcohol Dehydrogenase or an Aldehyde Dehydrogenase

The genes encoding the leucine dehydrogenases LeuDH_{Pa} from Pseudomonas aeruginosa, the leucine dehydrogenase LeuDH_{Bc} from Bacillus cereus, the L-amino acid deaminase LAAD_{Pv} from Proteus vulgaris, the alcohol dehydrogenase Adh2 from S. cerevisae, the alcohol dehydrogenase YqhD from E. coli Nissle and the aldehyde dehydrogenase PadA from E. coli K12 were incorporate into the pTet-kivD plasmid described herein by Gibson assembly to generate the following constructs: pTet-kivD-leuDH_{Pa}, pTet-kivD-adh2, pTet-LeuDH_{Pa}-kivD-adh2, pTet-LeuDH_{Bc}-kivD-adh2, pTet-LeuDH_{Pa}-kivD-yqhD, pTet-LeuDH_{Bc}-kivD-yqhD, pTet-LeuDH_{Pa}-kivD-padA, pTet-LeuDH_{Bc}-kivD-padA, pTet-Laad_{Pv}-kivD-adh2, pTet-Laad_{Pv}-kivD-yqhD, pTet-Laad_{Pv}-kivD-padA. Those constructs were transformed into the following E. coli Nissle strains described herein: ΔleuE, ΔleuE lacZ:tet-livKHMGF and ΔleuE ΔilvC lacZ:tet-livKHMGF

### Example 13. Improved Degradation of Leucine in Recombinant Bacteria Expressing Branched Chain Amino Acid Catabolism Enzyme by Expressing an Importer of Branched Chain Amino Acid

In these studies, all incubations were performed at 37° C. Cultures of E. coli Nissle ΔleuE lacZ:Tet-livKHMGF and Nissle ΔleuE lacZ:Tet-livKHMGF, pTet-kivD were grown overnight LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD and LivKHFMG and bacteria were grown for another 3 hours. Bacteria were then pelleted, washed, and resuspended in minimal media to an OD₆₀₀ of 1 and supplemented with 0.5% glucose and 2 mM leucine. Aliquots were removed at 0 h and 4 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

Isoleucine: 132.1/86.2

Valine: 118.1/72

The rate of leucine degradation was calculated based on the number of CFUs (colony forming units) determined at T0 by plating serial dilution on LB plates.

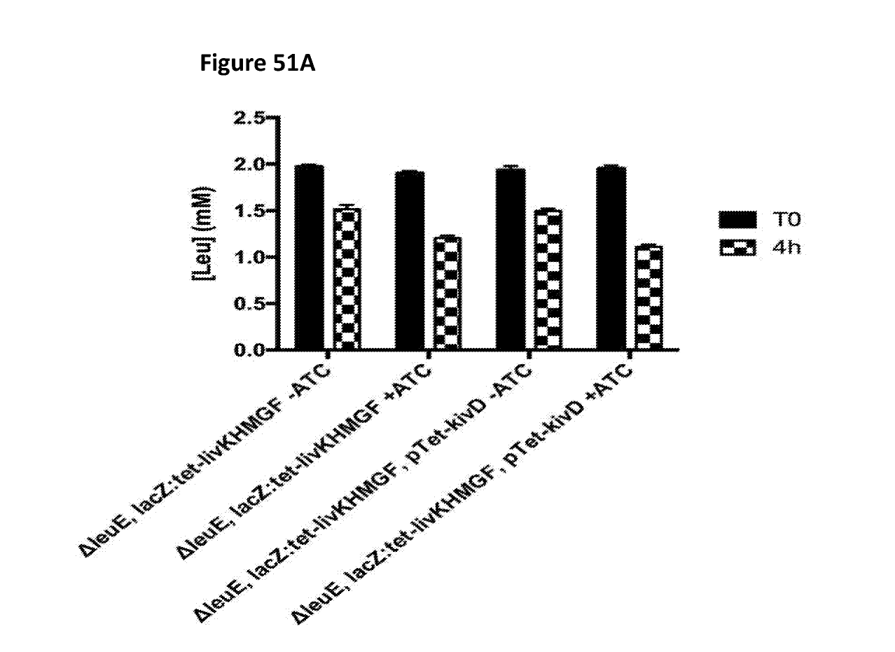

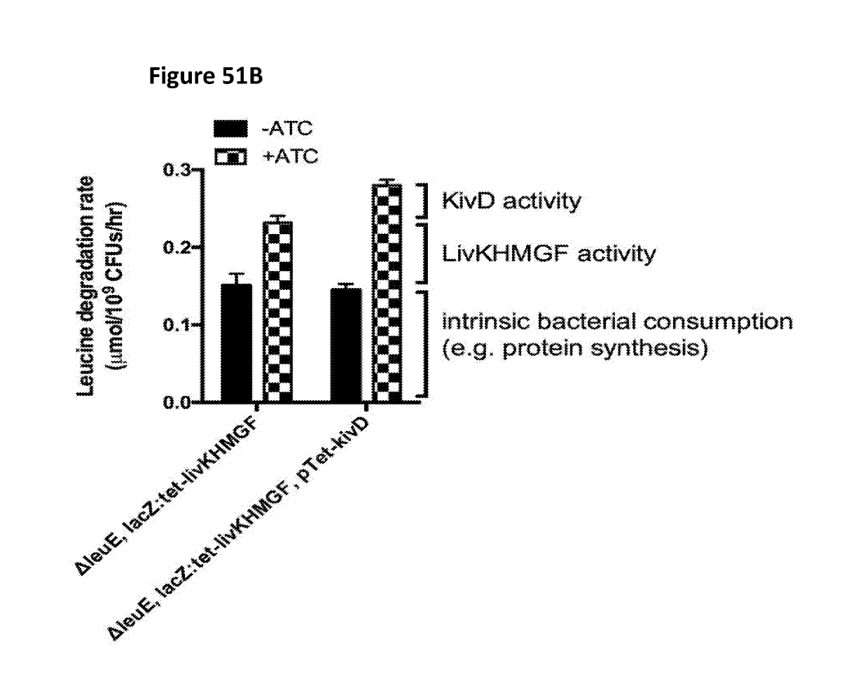

As shown in FIGS. 51A and 51B, leucine is consumed without the presence of ATC, due to normal bacterial growth during the assay. In the presence of ATC, degradation is further improved by the expression of livKHMGF and kivD.

### Example 14. Degradation of all Three Branched Chain Amino Acids by Recombinant Bacteria Expressing Branched Chain Amino Acid Catabolism Enzyme and Improved Degradation of Leucine by Expressing a Leucine Dehydrogenase

In these studies, all incubations were performed at 37° C. Cultures of E. coli Nissle ΔleuE lacZ:Tet-livKHMGF with the pTet-kivD or pTet-kivD-leuDH_{Pa} plasmid, were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD (SEQ NO:2), LeuDH_{Pa} (SEQ ID NO: 20) and LivKHMGF, and bacteria were grown for another 3 hours. Bacteria were then pelleted, washed, and resuspended in minimal media to OD₆₀₀ of 1, and supplemented with 0.5% glucose and the three branched chain amino acids (leucine, isoleucine and valine, 1 mM each). Aliquots were removed at 0 h, 3 h, 19 h for leucine, isoleucine and valine quantification by liquid chromatography-mass spectrometry (LCMS) using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

Isoleucine: 132.1/86.2

Valine: 118.1/72

The rate of leucine degradation was calculated based on the number of CFUs (colony forming units) determined at T0 by plating serial dilution on LB plates.

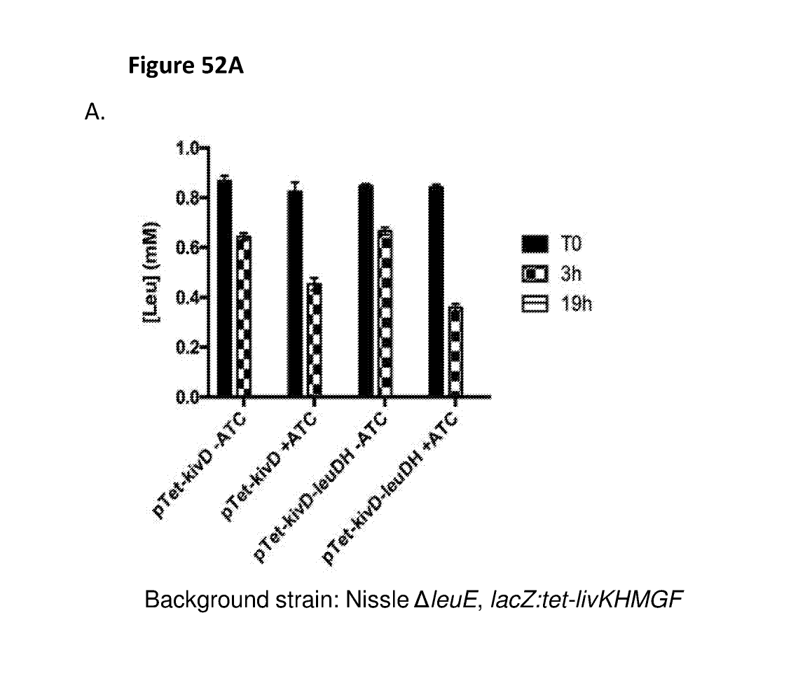

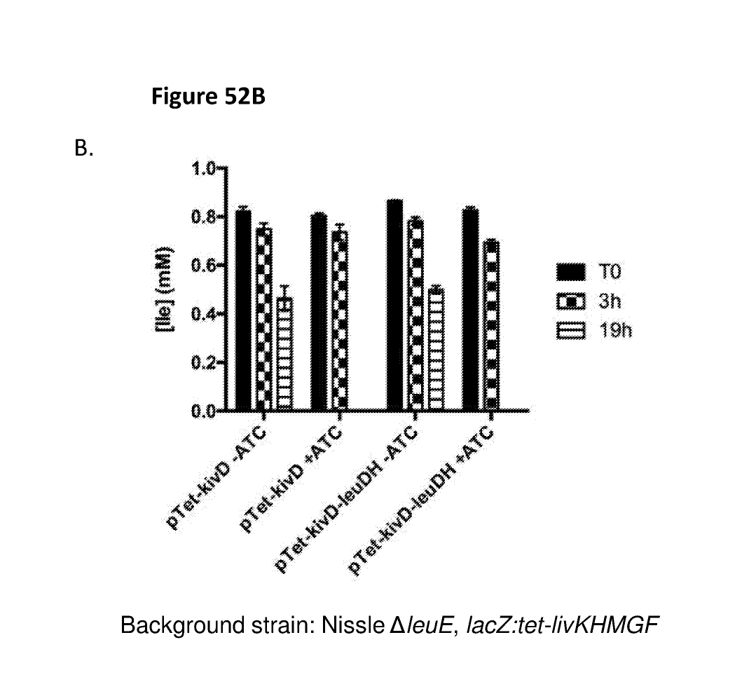

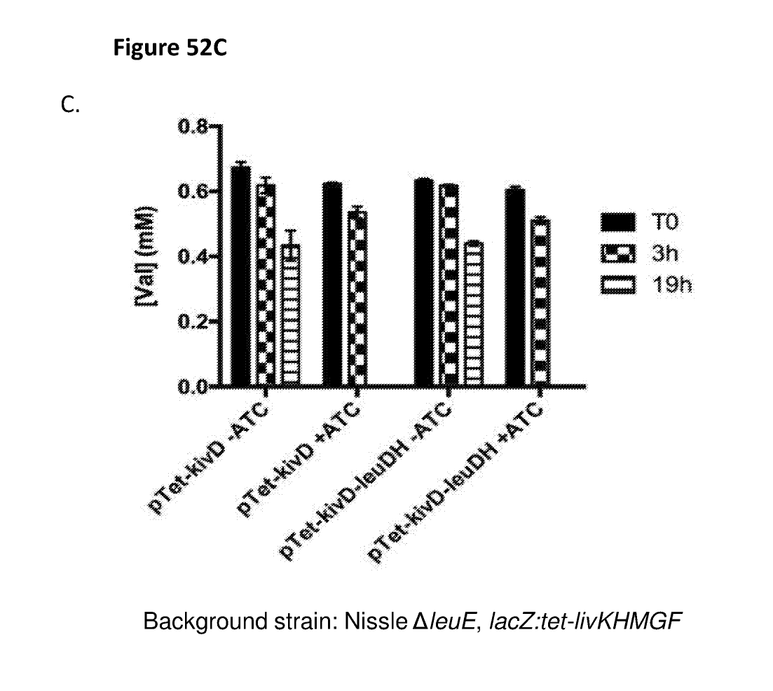

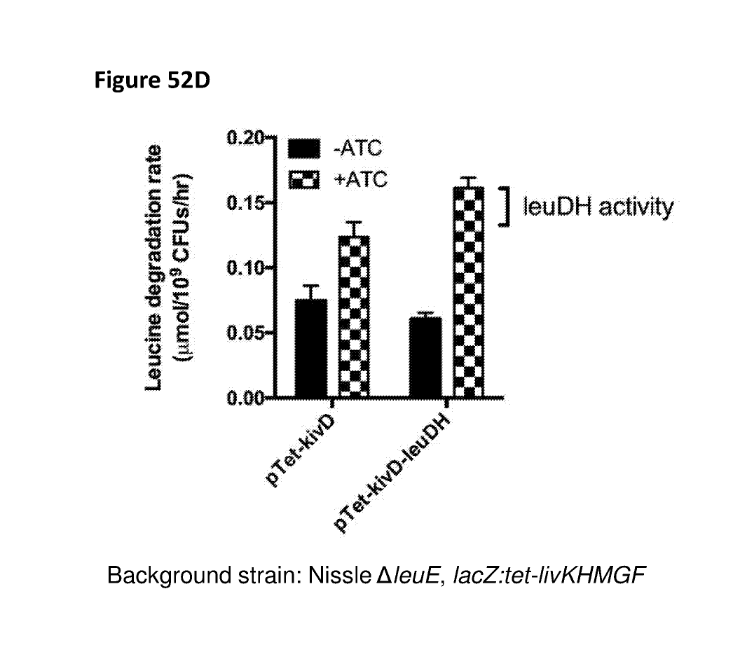

As shown in FIGS. 52A, 52B and 52C, leucine, isoleucine and valine were all degraded by the expression of kivD and kivD-leuDH_{Pa} in E. coli Nissle. The efficiency of leucine degradation was improved 25% by expressing the leucine dehydrogenase leuDH_{Pa} (FIG. 52D).

### Example 15. Enhanced Degradation of Leucine by Recombinant Bacteria Expressing an L-Amino Acid Deaminase

In these studies, all incubations were performed at 37° C. Cultures of E. coli Nissle ΔleuE ΔilvC lacZ:Tet-livKHMGF (SYN469) with the pTet-ilvE-kivD-adh2, pTet-LeuDH_{Pa}-kivD-adh2 or pTet-Laad_{Pv}-kivD-leuDHₚₐ plasmid, were grown overnight in LB for SYN469 and 50 ug/mL Kanamycin for strains containing a plasmid and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of KivD, LeuDH_{Pa}, IlvE, LAAD_{Pv}, and LivKHFMG, and bacteria were grown for another 3 hours. Bacteria were then pelleted, washed, and resuspended in minimal media to OD₆₀₀ of 1, and supplemented with 0.5% glucose and 2 mM. Aliquots were removed at 0 h and 3 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min. 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

The rate of leucine degradation was calculated based on the number of CPUs (colony forming units) determined at T0 by plating serial dilution on LB plates.

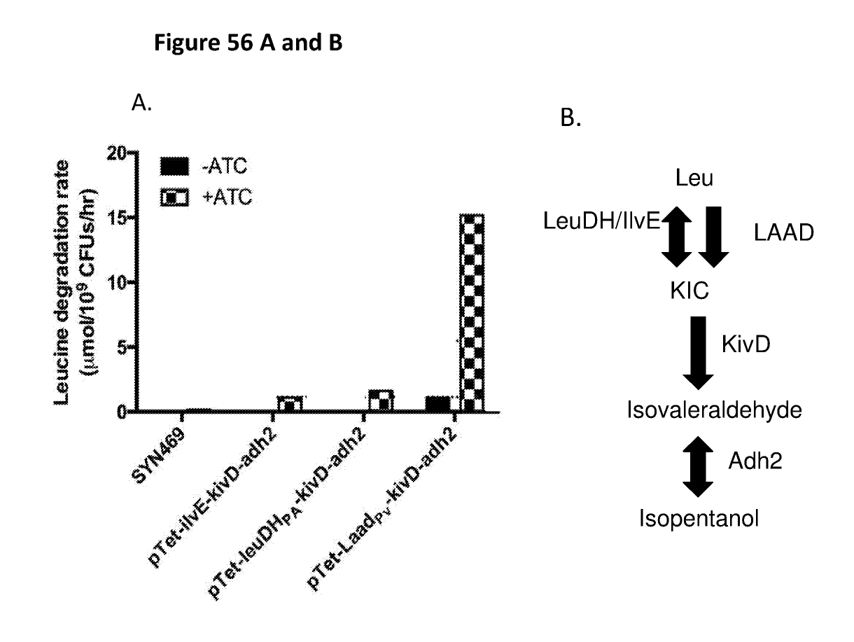

FIG. 56B depicts the leucine degradation pathway used in the strains tested. As shown in FIG. 56A, leucine degradation is greatly enhanced by the expression of LAAD_{Pv} (15-fold).

### Example 16. Degradation of Leucine by Recombinant Bacteria Expressing L-Amino Acid Deaminases from Proteus vulgaris and Proteus mirabilis

The gene encoding the L-amino acid deaminase Pma from Proteus mirabilis LAAD_{Pm} was cloned under the control of the tet promoter in the high copy plasmid pUC57-Kan to generate the pTet-Laad_{Pm} plasmid. The pTet-Laad_{Pm} plasmid was transformed in the ΔleuE ΔilvC lacZ:Tet-livKHMGF (SYN469). In these studies, all incubations were performed at 37° C. Cultures of E. coli Nissle ΔleuE ΔilvC lacZ:Tet-livKHMGF (SYN469) with the pTet-Laad_{Pv}-kivD-adh2, pTet-Laad_{Pv}-kivD-yqhD, pTet-Laad_{Pv}-kivD-padA or pTet-Laad_{Pm} plasmid, were grown overnight in LB for SYN469 and 50 ug/mL Kanamycin for strains containing a plasmid and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of the constructs, and bacteria were grown for another 3 hours. Bacteria were then pelleted, washed, and resuspended in minimal media to OD₆₀₀ of 1, and supplemented with 0.5% glucose and 2 mM leucine. Aliquots were removed at 0 h and 3 h for leucine quantification by liquid chromatography-mass spectrometry (LCMS) using a Thermo TSQ Quantum Max triple quadrupole instrument. Briefly, 100 uL aliquots were centrifuged at 4,500 rpm for 10 min. 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used are:

Leucine: 132.1/86.2

L-leucine-5,5,5-d₃: 135.1/89.3

The rate of leucine degradation was calculated based on the number of CFUs (colony forming units) determined at T0 by plating serial dilution on LB plates.

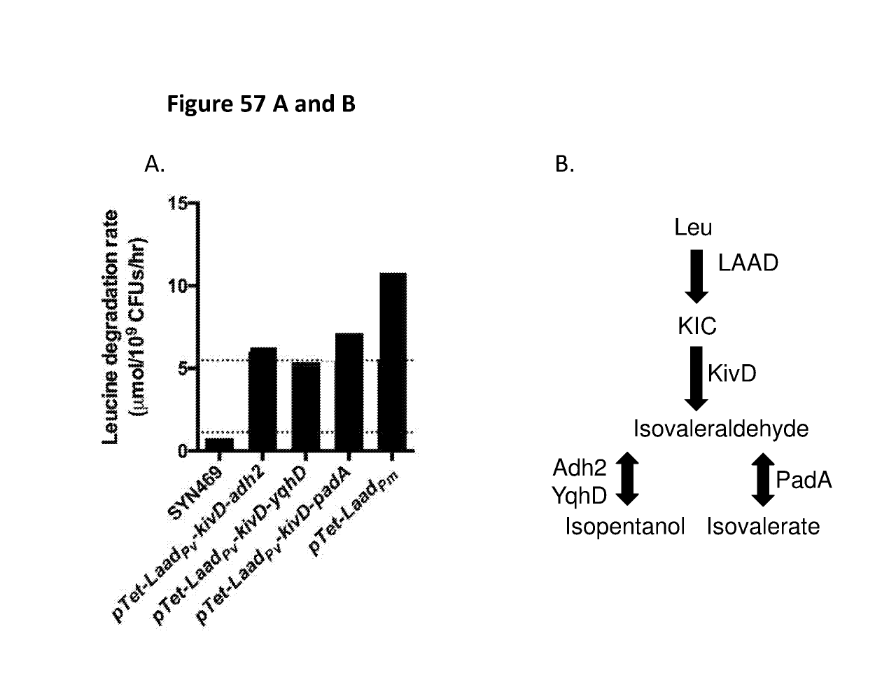

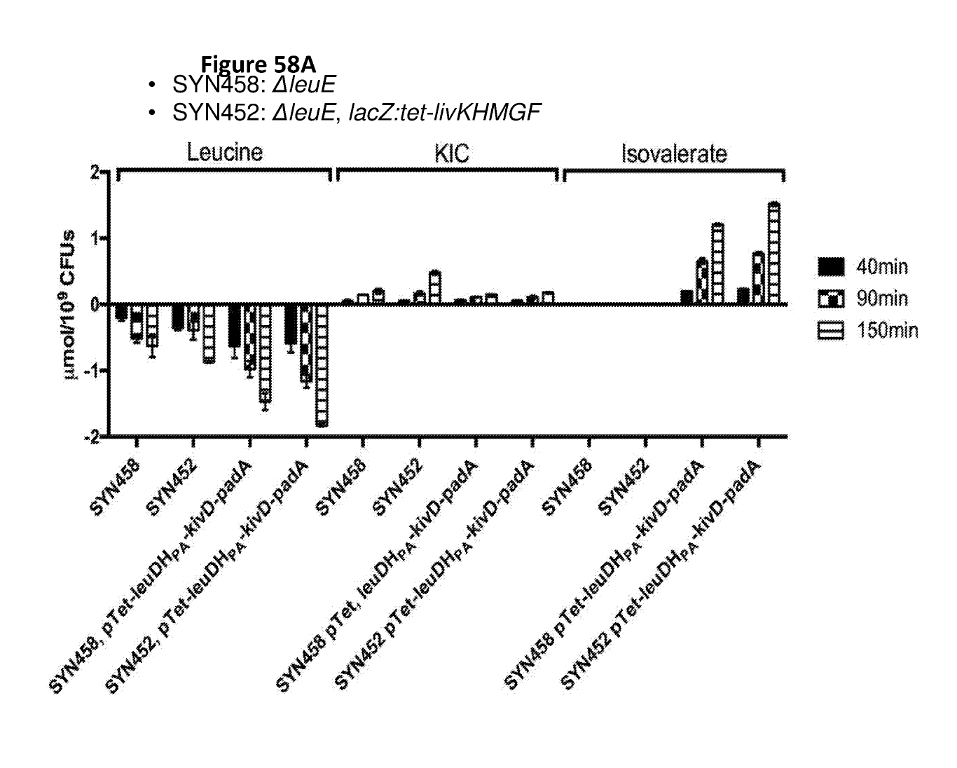

FIG. 57B depicts the leucine degradation pathway used in the strains tested. As shown in FIG. 57A, leucine degradation occurs at very efficient rates in strains expressing either LAAD_{Pv} or LAAD_{Pm}.

### Example 17. Improvement of Leucine Degradation by Recombinant Bacteria Expressing BCAA Catabolism Enzymes and a Leucine Importer

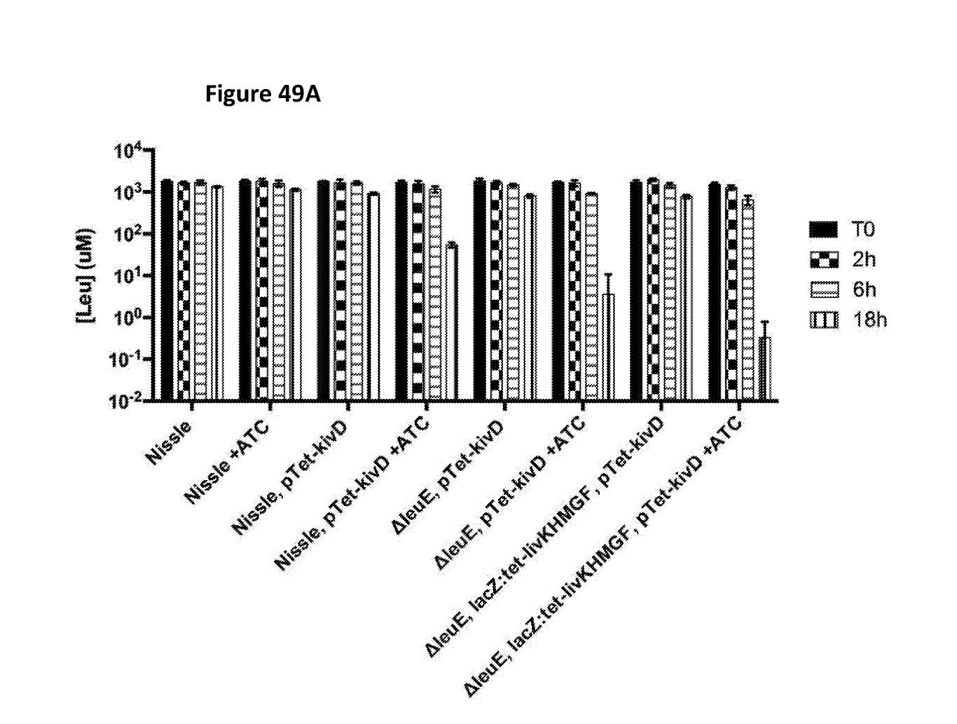

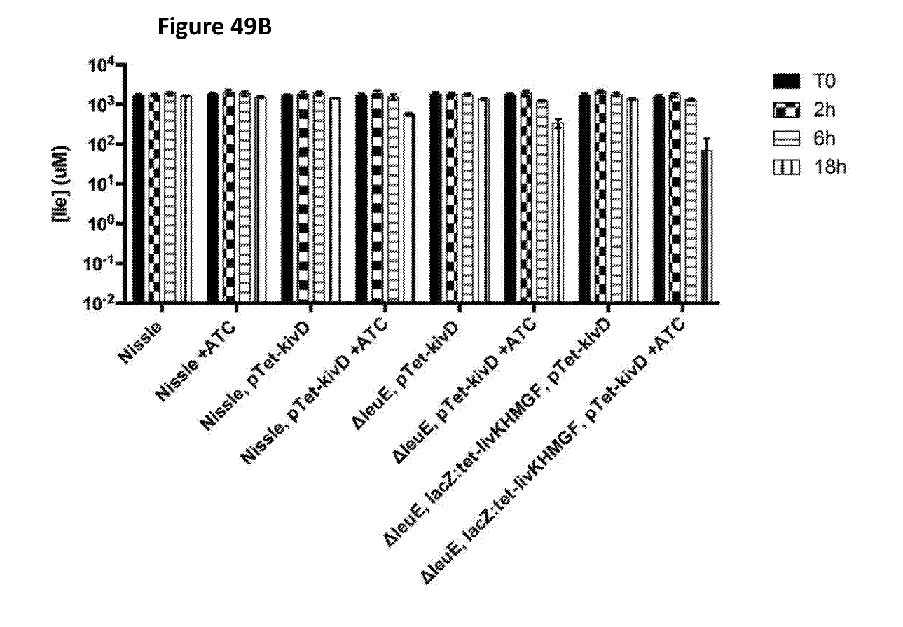

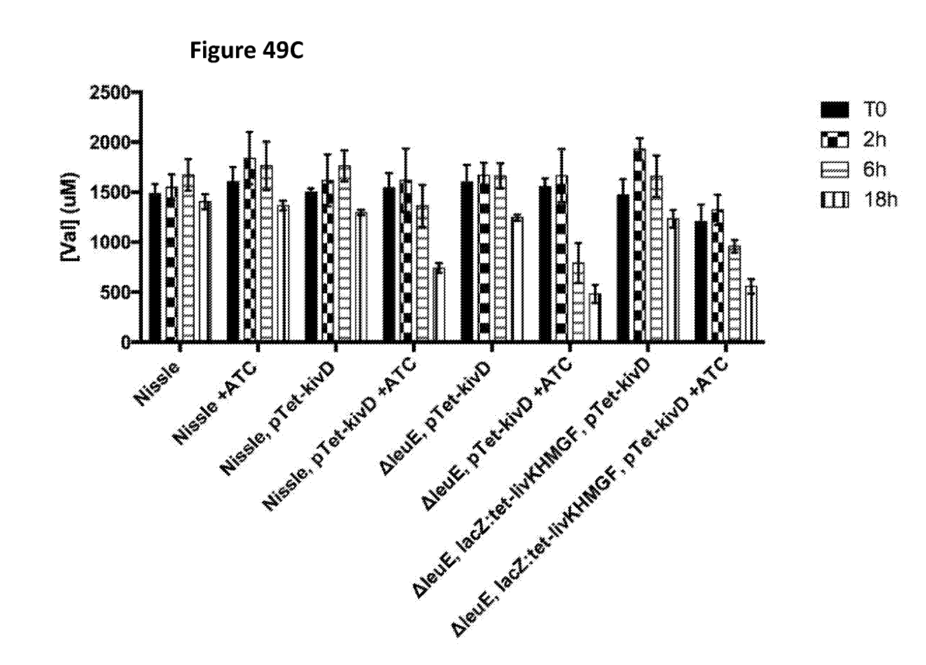

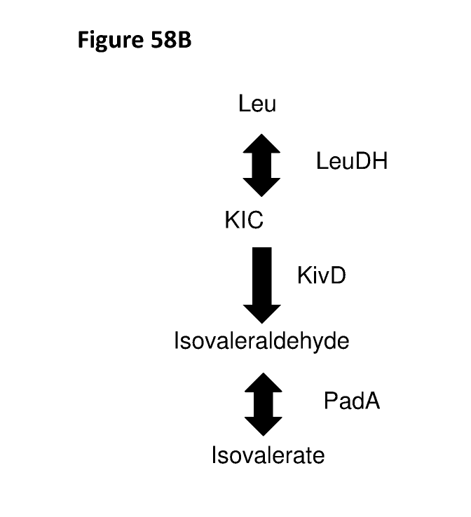

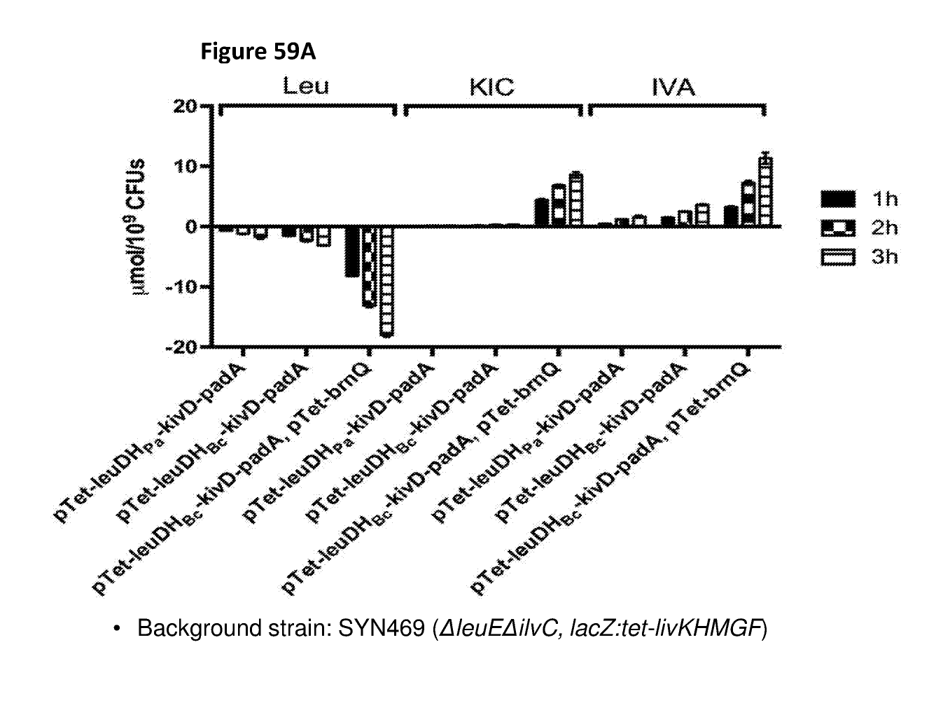

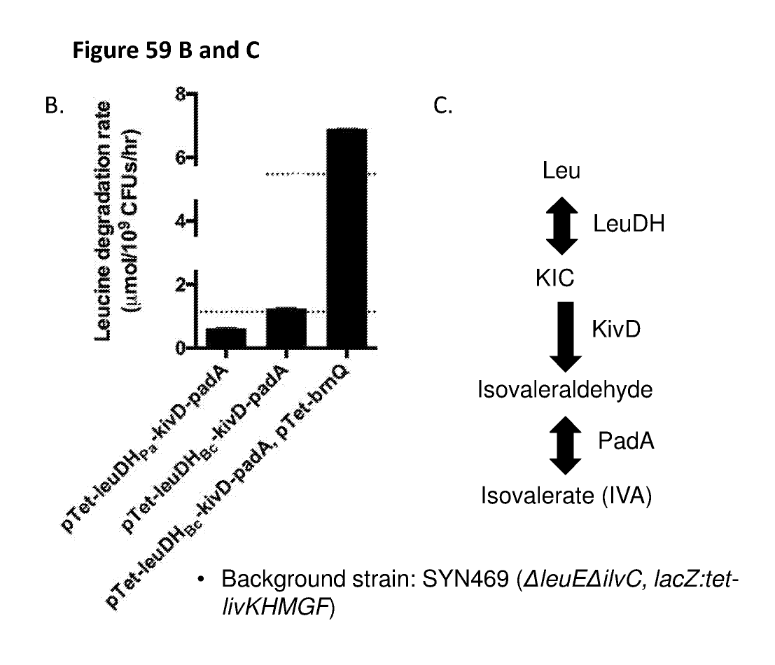

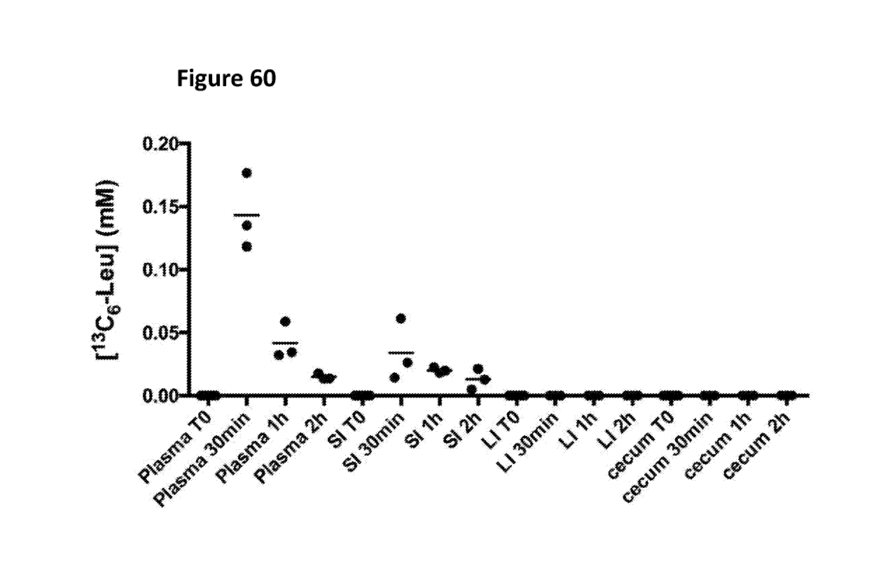

In these studies, all incubations were performed at 37° C. Cultures of E. coli Nissle ΔleuE lacZ:Tet-livKHMGF (SYN452) and ΔleuE (SYN458) with or without the pTet-LeuDH_{Pa}-kivD-padA plasmid, were grown overnight in LB for SYN452 and SYN458 or LB with 50 ug/mL Kanamycin for strains containing a plasmid and then diluted 1:100 in LB. The cells were grown with shaking (250 rpm) to early log phase with the appropriate antibiotics. Anhydrous tetracycline (ATC) was added to cultures at a concentration of 100 ng/mL to induce expression of the constructs, and bacteria were grown for another 3 hours. Bacteria were then pelleted, washed, and resuspended in minimal media, and supplemented with 0.5% glucose and 4 mM leucine. Aliquots were removed at T0, 40 min, 90 min and 150 min for leucine, KIC and isovaleric acid (IVA) quantification by liquid chromatography-mass spectrometry (LCMS) using a Thermo TSQ Quantum Max triple quadrupole instrument. The rate of leucine degradation, KIC and IVA production was calculated based on the number of CFUs (colony forming units) determined at T0 by plating serial dilution on LB plates. FIG. 49B depicts the leucine degradation pathway used in the strains tested. As shown in FIG. 58A, the expression of livKHMGF in SYN452 moderately improves the rate of leucine degradation in comparison with SYN458. This correlates with a mild increase in the production of isovalerate.

### Example 18. Improvement of Leucine Degradation by Recombinant Bacteria Expressing the Leucine Dehydrogenase from Bacillus cereus and the Low Affinity BCAA Transporter BrnQ

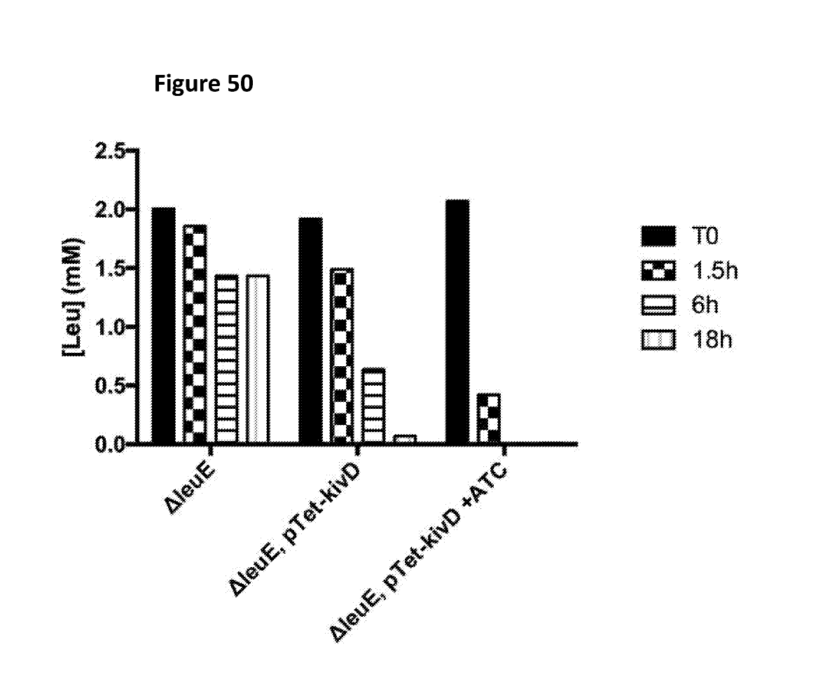

The gene encoding the leucine dehydrogenase from Bacillus cereus (LeuDHBc) (SEQ ID NO: 58) was cloned in place of the leucine dehydrogenase from Pseudomonas aeruginosa leuDHPa in the pTet-leuDHPa-kivD-padA constructs by Gibson assembly to generate the pTet-leuDHBc-kivD-padA plasmid. This plasmid was transformed into the E. coli Nissle ΔleuE ΔilvC lacZ:Tet-livKHMGF (SYN469) strain. The gene encoding E. coli Nissle low-affinity transporter BrnQ was cloned under the control of the tet promoter in the low-copy plasmid pSC101 by Gibson assembly. The generated pTet-brnQ plasmid was transformed into the newly generated E. coli Nissle ΔleuEΔilvC, lacZ:Tet-livKHMGF, pTet-leuDHBc-kivD-padA strain to generated the ΔleuEΔilvC, lacZ:Tet-livKHMGF, pTet-leuDHBc-kivD-padA, pTet-brnQ strain. In these studies, all incubations were performed at 37° C. Cultures of E. coli Nissle ΔleuEΔilvC,lacZ:Tet-livKHMGF, pTet-leuDHPa-kivD-padA, E. coli Nissle ΔleuEΔilvC,lacZ:Tet-livKHMGF, pTet-leuDHBc-kivD-padA and E. coli Nissle ΔleuEΔilvC,lacZ:Tet-livKHMGF, pTet-leuDHBc-kivD-padA,pTet-brnQ strains were grown overnight in LB with 50 ug/mL Kanamycin and 100 ug/mL carbenicillin for the for strain containing pTet-brnQ. The cells were grown with shaking (250 rpm) to early log phase with the appropriate antibiotics. Anhydrous tetracycline (ATC) was added to cultures at a concentration of 100 ng/mL to induce expression of the constructs, and bacteria were grown for another 3 hours. Bacteria were then pelleted, washed, and resuspended in minimal media, and supplemented with 0.5% glucose and 4 mM leucine. Aliquots were removed at T0, 1 h, 2 h and 3 h for leucine, KIC and isovaleric acid (IVA) quantification by liquid chromatography-mass spectrometry (LCMS) using a Thermo TSQ Quantum Max triple quadrupole instrument. The rate of leucine degradation, KIC and IVA production was calculated based on the number of CFUs (colony forming units) determined at T0 by plating serial dilution on LB plates. FIG. 50C depicts the leucine degradation pathway used in the strains tested. As shown in FIG. 50A, the expression of leuDHBc doubles the rate of leucine degradation compare to leuDHPa. The expression of the low-affinity BCAA transporter BrnQ dramatically improves the rate of leucine degradation, by 4 to 5 fold. In both cases, the increased level of leucine degradation correlates with an increased level of isovalerate production as shown in FIG. 50A. The expression of BrnQ also leads to the accumulation of KIC, suggesting that the decarboxylation of KIC by kivD becomes the limiting step in the pathway.

### Example 19. Recirculation of Isotopic Leucine into the Mouse Intestine after Subcutaneous Injection

To understand the kinetic relationship between intestinal and systemic levels of exogenously administered leucine, heavy isotope-labeled leucine (¹³C₆) was injected subcutaneously at 0.1 mg/g in BL6 mice and quantified in plasma, small intestine effluent, cecum and large intestine effluent at different times after injection (before injection (T0), 30 min, 1 h and 2 h after injection). For each time point, 3 mice were bled and dissected to collect their small intestine, cecum and large intestine content. ¹³C₆-Leu was quantified by LC-MS/MS by liquid chromatography-mass spectrometry (LCMS) using a Thermo TSQ Quantum Max triple quadrupole instrument. Briefly, 10 uL of samples were resuspended in 90 uL of derivatization mix (50 mM 2-Hydrazinoquinoline, 50 mM triphenylphosphine, 50 mM, 2,2′-dipyridyl disulfide in acetonitrile) with 1 ug/mL L-leucine-5,5,5-d₃ (isotope used as internal standard). The samples were then incubated at 60° C. for 1 h, centrifuged at 4,500 rpm at 4° C. for 5 min 20 uL was then transferred to 180 uL of water, 0.1% formic acid and placed in the LCMS autosampler. A C18 column 50×2 mm, Sum particles was used (Luna, Phenomenex). The mobile phases used were water 0.1% formic acid (solvent A) and acetonitrile 0.1% (solvent B). The mass spectrometer was run in positive mode and the Q1/Q3 transitions used for ¹³C₆-Leu quantification were 279.1/144.2 and 279.1/160.2.

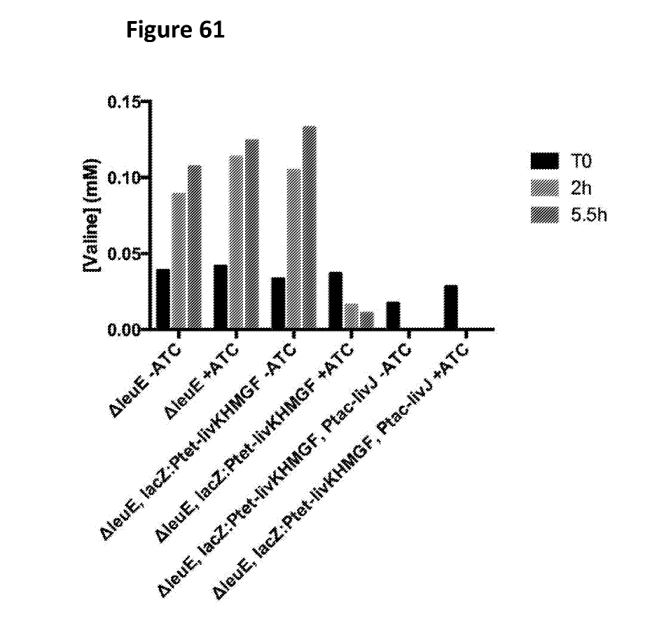

FIG. 61 shows that ¹³C₆-Leu is present in the plasma and the small intestine as early as 30 min after injection, demonstrating that leucine is able to recirculate from the periphery into the small intestine. After 30 min, the level gradually decreases. ¹³C₆-Leu remains undetectable in the cecum and the large intestine, suggesting that leucine is not able to recirculate to those parts of the gastrointestinal tract. Those results demonstrate that an increase in plasma leucine level, mimicking a transient acute MSUD state, can be obtained by subcutaneous injection of leucine and that part of this leucine can become available for an engineered BCAA-degrading bacteria residing in the gastrointestinal tract.

### Example 20. Increase of BCAA Import by Overexpressing the High Affinity BCAA Transporters livKHMGF and livJHMGF in Vitro

In these studies, all the strains are derived from the human probiotic strain E. coli Nissle ΔleuE. In the ΔleuE, lacZ:Ptet-livKHMGF strain, the endogenous promoter of livJ was swapped with the constitutive promoter Ptac by lambda-red recombination using the Ptac-livJ construct (SEQ ID NO: 11) to generate the ΔleuE, lacZ:Ptet-livKHMGF, Ptac-livJ strain. In this strain, livJ is constitutively induced. In the presence of ATC, both BCAA transporters livKHMGF and livJHMGF are expressed. ΔleuE; ΔleuE, lacZ:Ptet-livKHMGF; ΔleuE, lacZ:Ptet-livKHMGF, Ptac-livJ strains were grown overnight at 37° C. and 250 rpm in 4 mL of LB. Bacterial Cells were then diluted 100 fold in 4 mL LB and grown for 2 h at 37° C. and 250 rpm. Cells were then split in two 2 mL culture tubes. One 2 mL culture tube was induced with 100 ng/mL anhydrotetracycline (ATC) to activate the Ptet promoter. After 1 h induction, 1 mL of cells was spun down at maximum speed for 30 seconds in a microcentrifuge. The supernatant was then removed and the pellet re-suspended in 1 mL M9 medium 0.5% glucose. The cells were spun down again at maximum speed for 30 seconds and resuspended in 1 mL M9 medium 0.5% glucose. The cells were then transferred to a culture tube and incubated at 37° C. and 250 rpm for 5.5 h. 150 μL of cells were collected at 0 h, 2 h and 5.5 h and the concentration of valine in the cell supernatant at the different time points was determined by LC-MS/MS using a Thermo TSQ Quantum Max triple quadrupole instrument. Briefly, 100 uL aliquots were centrifuged at 4,500 rpm for 10 min 10 uL of the supernatant was resuspended in 90 uL water with 1 ug/mL L-leucine-5,5,5-d₃ (isotope used as internal standard). 10 uL of the samples was then resuspended in water, 0.1% formic acid and placed in the LCMS autosampler. A C18 column 100×2 mm, 3 um particles was used (Luna, Phenomenex). The mobile phases used were water 0.1% formic acid (solvent A) and acetonitrile 0.1% (solvent B). The gradient used was:

0 min: 100% A, 0% B

0.5 min: 100% A, 0% B

1.5 min: 10% A, 90% B

3.5 min: 10% A, 90% B

3.51 min: 100% A, 0% B

4.5 min: 100% A, 0% B

The Q1/Q3 transitions used is:

Valine: 118.1/72

As FIG. 61 shows, the natural secretion of valine by E. coli Nissle is observed for the ΔleuE strain. The secretion of valine is strongly reduced for ΔleuE, lacZ:Ptet-livKHMGF in the presence of ATC. This strongly suggests that the secreted valine is efficiently imported back into the cell by livKHMGF. The secretion of valine is abolished in the ΔleuE, lacZ:Ptet-livKHMGF, Ptac-livJ strain, with or without ATC. This strongly suggests that the constitutive expression of livJ is sufficient to import back the entire amount of valine secreted by the cell via the livJHMGF transporter. In conclusion, we successfully engineered E. coli Nissle to efficiently import BCAA, in this case valine, using both an inducible promoter (Ptet), and a constitutive promoter (Ptac), controlling the expression of livKHMGF and livJ respectively.

### Example 21. Improved Transport of Leucine in Recombinant Bacteria Expressing a Leucine Importer

In order to test if expressing the high-affinity leucine transporter livKHMGF increases the transport of leucine into the bacterial cell, the minimum inhibitory concentration (MIC) of the toxic analog 3-fluoroleucine was determined for the following E. coli Nissle strains: E. coli Nissle, ΔleuE and ΔleuE, lacZ:Tet-livKHMGF. Those strains were grown overnight in LB and diluted 2,000 fold in M9 minimum media supplemented with 0.5% glucose, in the presence of 250, 125, 62.5, 31.2, 15.6, 7.8, 3.9, 2, 1 or 0 ug/mL 3-fluoroleucine in the presence or absence of 100 ng/mL ATC for ΔleuE, lacZ:Tet-livKHMGF. Cells were grown at 37° C. for 20 h. The MIC for each strain, with our without ATC, was determined by looking at the presence or absence of bacterial growth for each treatment and was defined as the minimum concentration blocking bacterial growth. The following Table 15 describes the results:

MIC (ug/mL)

| Strain | −ATC | +ATC |
|---|---|---|
| Nissle | 31.25 | ND |
| ΔleuE | 62.5 | ND |
| ΔleuE, lacZ:Tet-livKHMGF | 31.25 | 2 |

The induction of the leucine importer livKHMGF by ATC in the ΔleuE, lacZ:Tet-livKHMGF strain led to a 16-fold reduction in the MIC to 3-fluoroleucine, going from 31.25 to 2 ug/mL. This dramatic increase in sensitivity to the leucine toxic analog demonstrates that the expression of livKHMGF leads to a substantial increase in leucine transport into the cell.

(d) Example 22. In Vitro Activity of Leucine Consuming Strains (with or without a Low-Copy ATC-Inducible brnQ Construct)

To test the low-copy ATC-inducible constructs and confirm the effect of brnQ on leucine degradation, strains were generated (according to methods described in Example 1 and others) as follows and tested for in vitro leucine degradation activity. SYN1992 comprises ΔleuE, ΔilvC, a tet inducible livKHMGF construct integrated into the bacterial chromosome at the LacZ locus, and a tet inducible leuDH(Bc)-kivD-adh2-rrnB ter construct on a low copy plasmid (ΔleuE, ΔilvC, lacZ:tetR-Ptet-livKHMGF, tetR-Ptet-leuDH(Bc)-kivD-adh2-rrnB ter (pSC101)). SYN1980 comprises ΔleuE, ΔilvC, a tet-inducible livKHMGF construct integrated at the lacZ locus in the bacterial chromosome, and a tet-inducible leuDH(Bc)-kivD-adh2-brnQ-rrnB ter construct on a low copy plasmid (ΔleuE, ΔilvC, lacZ:tetR-Ptet-livKHMGF, tetR-Ptet-leuDH(Bc)-kivD-adh2-brnQ-rrnB ter (pSC101)). SYN469, comprising ΔleuE, ΔilvC, and integrated lacZ:tetR-Ptet-livKHMGF, was used as a control.

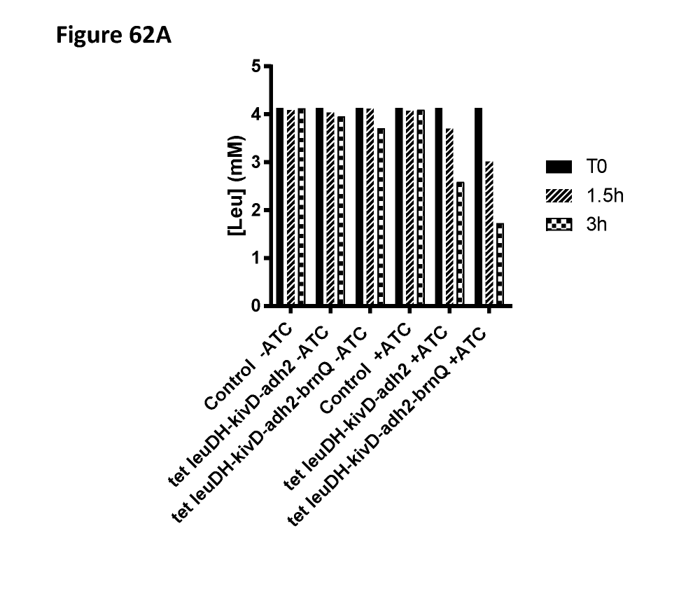

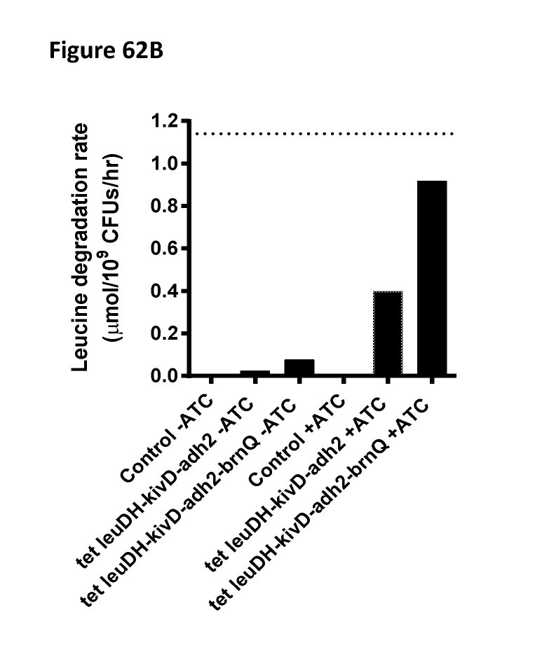

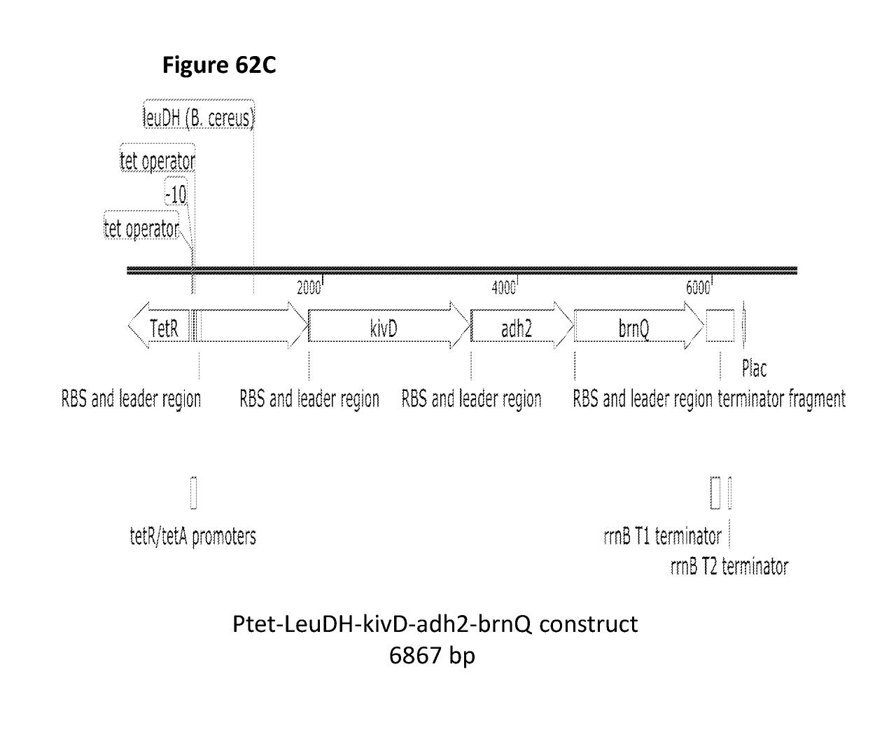

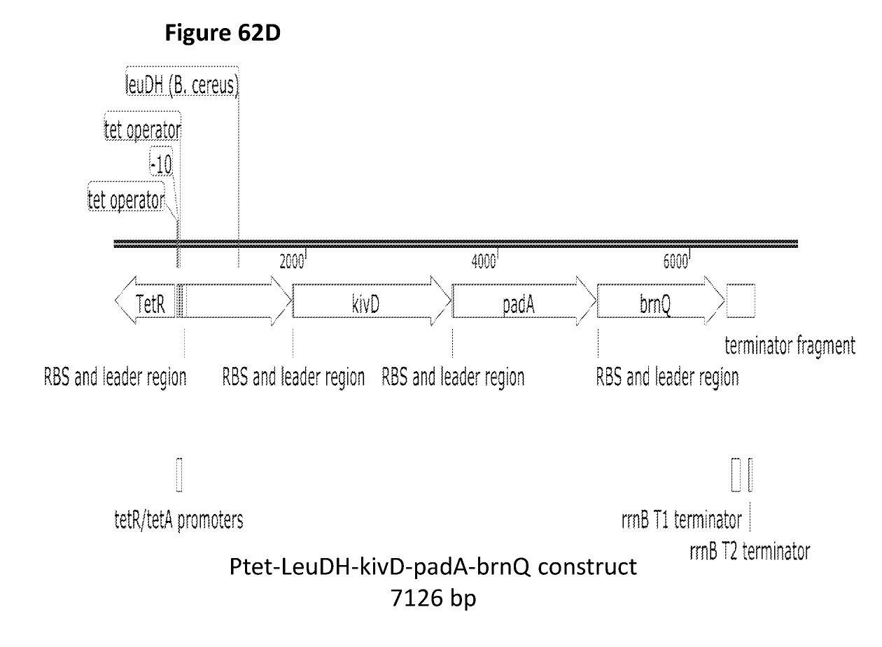

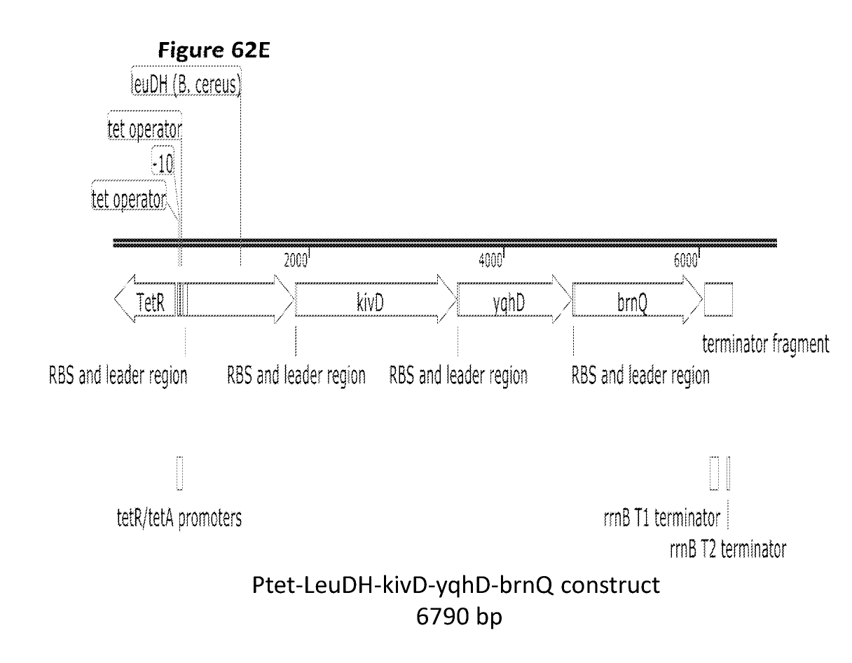

Overnight cultures were subcultured 1/100 in 5 mL LB plus carbenicillin (except for SYN469) and grown for 3 h at 37 C, 250 rpm. Cultures were either left uninduced or induced for 2 hours with ATC 100 ng/mL Bacteria (1 ml) were spun down, washed with 1 mL of M9 plus 0.5% glucose, and resuspended 1 mL of M9 medium with 0.5% glucose and 4 mM leucine. Bacteria concentration was determined using a cellometer. Bacteria were transferred to culture tubes (at 37 C, 250 rpm) and samples were taken at 1.5 and 3 h, leucine concentrations measured and degradation rates calculated. Results are shown in FIG. 62A and FIG. 62B. Leucine degradation was increased in both SYN1992 and SYN1980 upon addition of tetracycline, with SYN1980 (comprising tet-inducible BrnqQ) having a greater degradation rate.

### Example 23. In Vitro Activity of Leucine Consuming Strains (with or without a Low-Copy FNR-Inducible brnQ Construct)

To test low copy no/low oxygen inducible FNR driven constructs and confirm the effect of brnQ on leucine degradation, strains were generated (according to methods described in Example 1 and others) as follows and tested for in vitro Leucine degradation activity.

SYN1993 comprises ΔleuE, ΔilvC, a tetracycline inducible livKHMGF construct integrated into the LacZ locus of the bacterial chromosome, and a low/no oxygen inducible, FNR driven leuDH(Bc)-kivD-adh2-rrnB ter construct on a low copy plasmid (SYN1993: ΔleuE, ΔilvC, lacZ:tetR-Ptet-livKHMGF, PfnrS-leuDH(Bc)-kivD-adh2-rrnB ter (pSC101)). SYN1981 comprises ΔleuE, ΔilvC, a tetracycline inducible livKHMGF construct integrated into the LacZ locus of the bacterial chromosome, and a low/no oxygen inducible, FNR driven leuDH(Bc)-kivD-adh2-brnQ-rrnB ter construct on a low copy plasmid (SYN1981: ΔleuE, ΔilvC, lacZ:tetR-Ptet-livKHMGF, PfnrS-leuDH(Bc)-kivD-adh2-brnQ-rrnB ter (pSC101)). SYN469, comprising ΔleuE, ΔilvC, and integrated tetR-Ptet-livKHMGF at the LacZ locus, was used as a control.

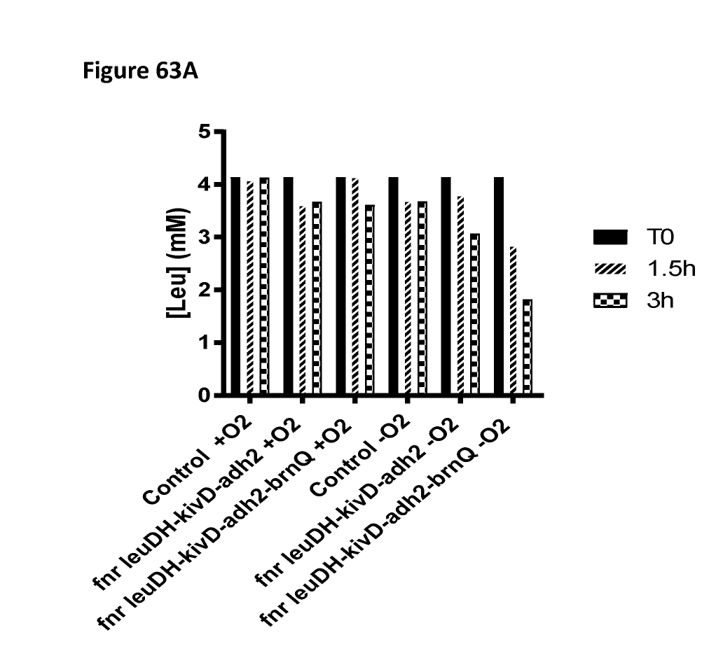

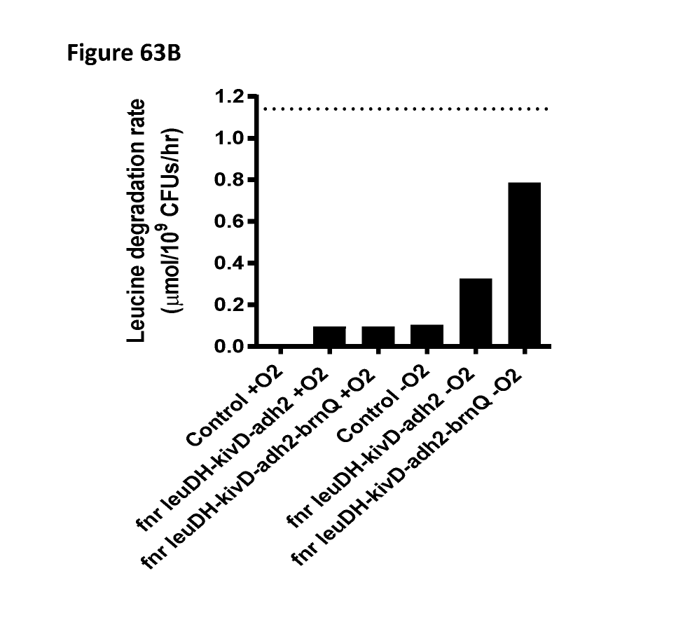

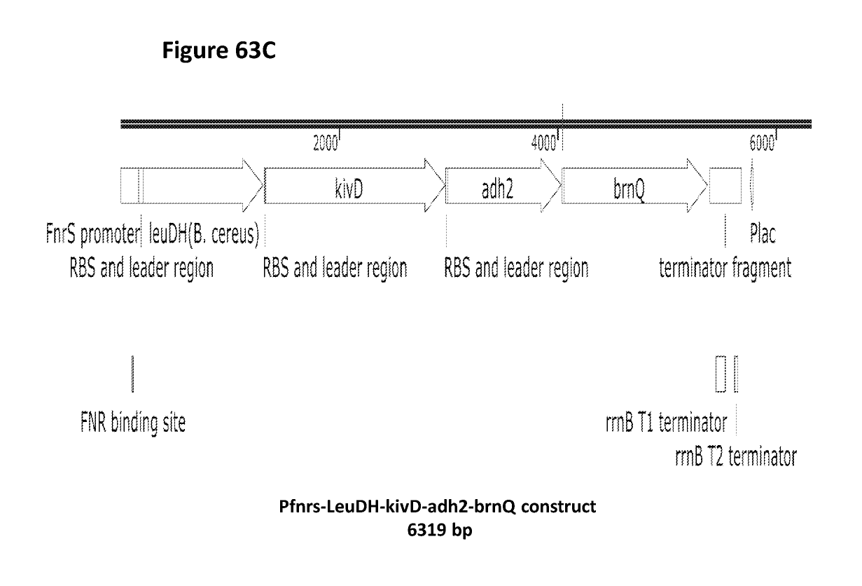

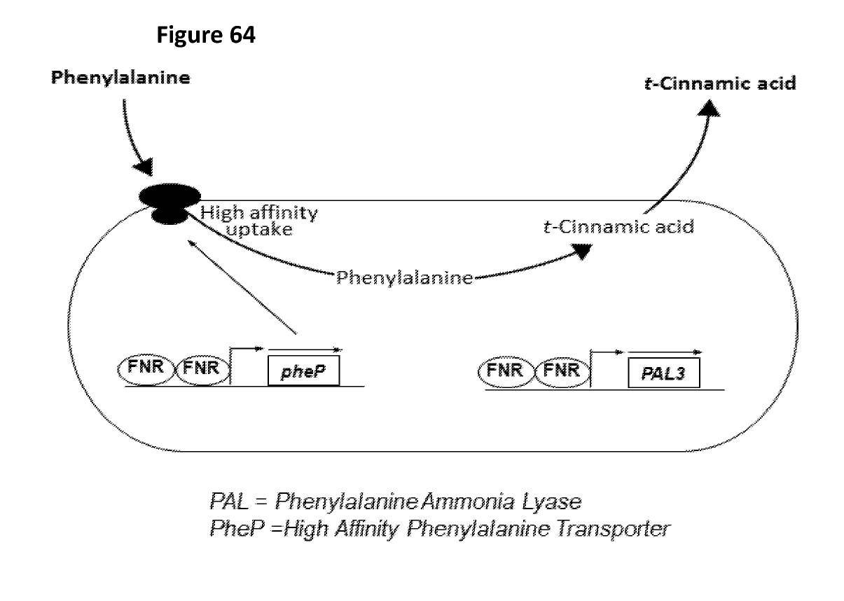

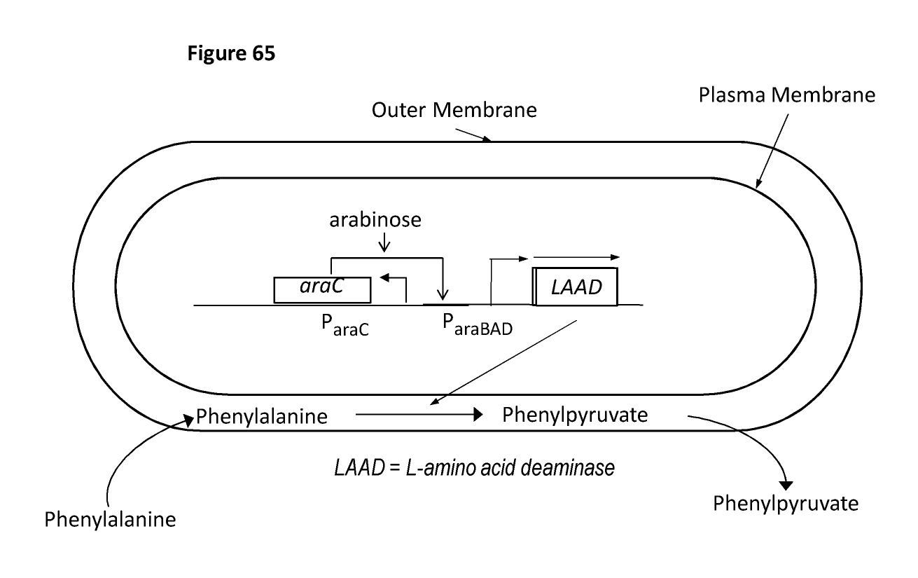

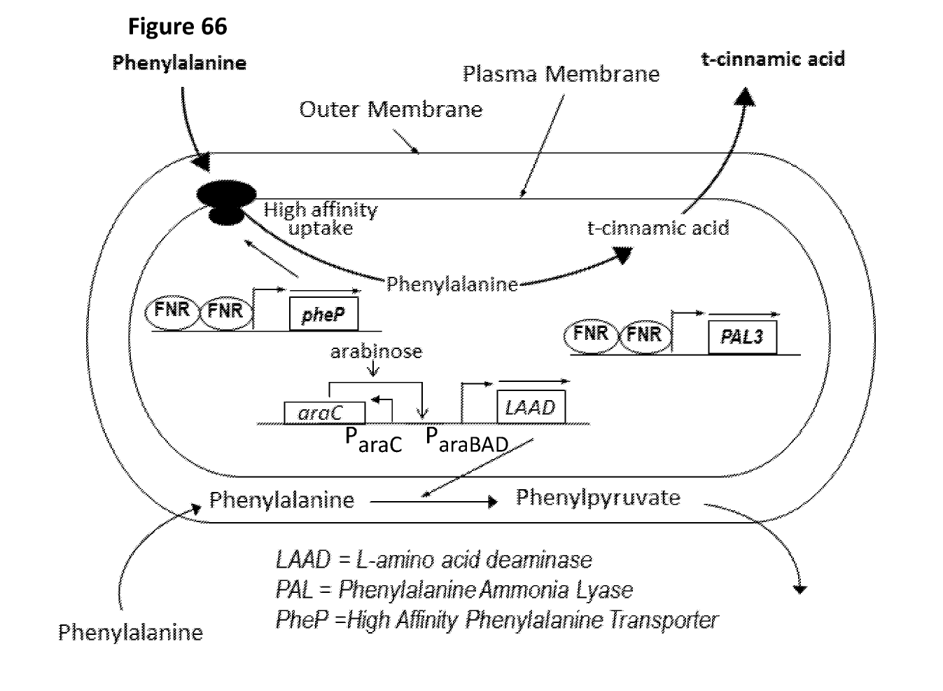

Overnight cultures were subcultured 1/100 in 5 mL LB plus carbenicillin (except for SYN469) and grown for 3 h at 37 C, 250 rpm. Cultures were either left uninduced or transferred to an Coy anaerobic chamber supplying 90% N₂, 5% CO₂, and 5% H₂. Bacteria (1 ml) were spun down, washed with 1 mL of M9 plus 0.5% glucose, and resuspended 1 mL of M9 medium with 0.5% glucose and 4 mM leucine. Bacterial concentration was determined using a cellometer. Bacteria were transferred to culture tubes (at 37 C, 250 rpm), samples were taken at 1.5 and 3 h and leucine concentrations measured and degradation rates calculated. Results are shown in FIG. 63A and FIG. 63B. Leucine degradation was increased in both SYN1993 and SYN1981 upon anaerobic induction, with SYN1981 (comprising FNR inducible BrnqQ) having a greater degradation rate.

### Example 24. Construction of PAL Plasmids

To facilitate inducible production of PAL in Escherichia coli Nissle, the PAL gene of Anabaena variabilis (“PAL1”) or Photorhabdus luminescens (“PAL3”), as well as transcriptional and translational elements, were synthesized (Gen9, Cambridge, Mass.) and cloned into vector pBR322. The PAL gene was placed under the control of an inducible promoter. Low-copy and high-copy plasmids were generated for each of PAL1 and PAL3 under the control of an inducible FNR promoter or a Tet promoter. However, as noted above, other promoters may be used to drive expression of the PAL gene, other PAL genes may be used, and other phenylalanine metabolism-regulating genes may be used.

### Example 25. Transforming E. coli

Each of the plasmids described herein was transformed into E. coli Nissle for the studies described herein according to the following steps. All tubes, solutions, and cuvettes were pre-chilled to 4° C. An overnight culture of E. coli Nissle was diluted 1:100 in 5 mL of lysogeny broth (LB) containing ampicillin and grown until it reached an OD₆₀₀ of 0.4-0.6. The E. coli cells were then centrifuged at 2,000 rpm for 5 min at 4° C., the supernatant was removed, and the cells were resuspended in 1 mL of 4° C. water. The E. coli were again centrifuged at 2,000 rpm for 5 min at 4° C., the supernatant was removed, and the cells were resuspended in 0.5 mL of 4° C. water. The E. coli were again centrifuged at 2,000 rpm for 5 min at 4° C., the supernatant was removed, and the cells were finally resuspended in 0.1 mL of 4° C. water. The electroporator was set to 2.5 kV. Plasmid (0.5 μg) was added to the cells, mixed by pipetting, and pipetted into a sterile, chilled cuvette. The dry cuvette was placed into the sample chamber, and the electric pulse was applied. One mL of room-temperature SOC media was added immediately, and the mixture was transferred to a culture tube and incubated at 37° C. for 1 hr. The cells were spread out on an LB plate containing ampicillin and incubated overnight.

### Example 26. Comparison of Phenylalanine Metabolism Between High-Copy and Low Copy Plasmids Expressing PAL1 and PAL2

Genetically engineered bacteria comprising the same PAL gene, either PAL3 on a low-copy plasmid or high copy plasmid (SYN-PKU101 and SYN-PKU102) or PAL3 on a low-copy plasmid or a high copy plasmid (SYN-PKU201 and SYN-PKU202) were assayed for phenylalanine metabolism in vitro.

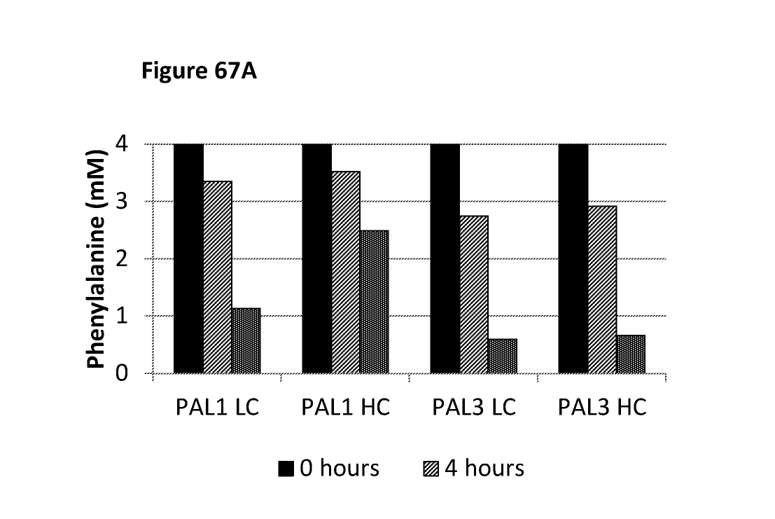

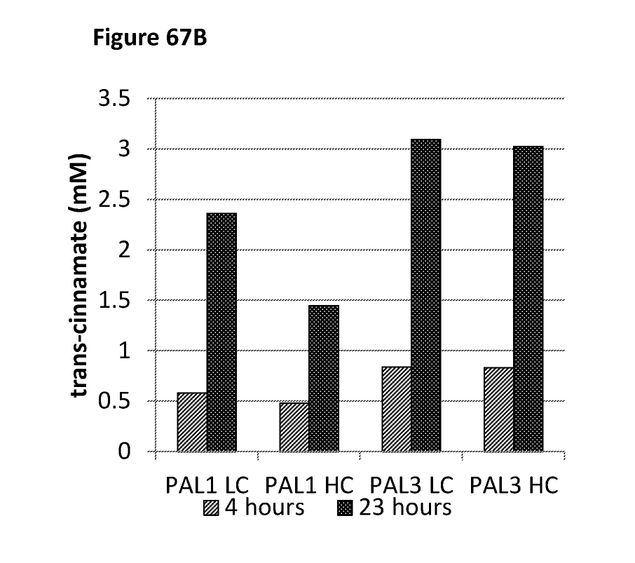

Engineered bacteria were induced with anhydrous tetracycline (ATC), and then grown in culture medium supplemented with 4 mM (660,000 ng/mL) of phenylalanine for 2 hours. Samples were removed at 0 hrs, 4 hrs, and 23 hrs, and phenylalanine (FIG. 67A) and trans-cinnamic acid(TCA) (FIG. 67B) concentrations were determined by mass spectrometry as described in Examples 24-26.

High copy plasmids and low copy plasmid strains were found to metabolize and reduce phenylalanine to similar levels. A greater reduction in phenylalanine levels and increase in TCA levels was observed in the strains expressing PAL3.

### Example 27. Phenylalanine Transporter—Integration of PheP into the Bacterial Chromosome

In some embodiments, it may be advantageous to increase phenylalanine transport into the cell, thereby enhancing phenylalanine metabolism. Therefore, a second copy of the native high affinity phenylalanine transporter, PheP, driven by an inducible promoter, was inserted into the Nissle genome through homologous recombination. The pheP gene was placed downstream of the Pₜₑₜ promoter, and the tetracycline repressor, TetR, was divergently transcribed. This sequence was synthesized by Genewiz (Cambridge, Mass.). To create a vector capable of integrating the synthesized TetR-PheP construct into the chromosome, Gibson assembly was first used to add 1000 bp sequences of DNA homologous to the Nissle lacZ locus into the R6K origin plasmid pKD3. This targets DNA cloned between these homology arms to be integrated into the lacZ locus in the Nissle genome. Gibson assembly was used to clone the TetR-PheP fragment between these arms. PCR was used to amplify the region from this plasmid containing the entire sequence of the homology arms, as well as the pheP sequence between them. This PCR fragment was used to transform electrocompetent Nissle-pKD46, a strain that contains a temperature-sensitive plasmid encoding the lambda red recombinase genes. After transformation, cells were grown for 2 hrs before plating on chloramphenicol at 20 μg/mL at 37° C. Growth at 37° C. cures the pKD46 plasmid. Transformants containing anhydrous tetracycline (ATC)-inducible pheP were lac-minus (lac-) and chloramphenicol resistant.

### Example 28. Effect of the Phenylalanine Transporter on Phenylalanine Degradation

To determine the effect of the phenylalanine transporter on phenylalanine degradation, phenylalanine degradation and trans-cinnamate accumulation achieved by genetically engineered bacteria expressing PAL1 or PAL3 on low-copy (LC) or high-copy (HC) plasmids in the presence or absence of a copy of pheP driven by the Tet promoter integrated into the chromosome was assessed.

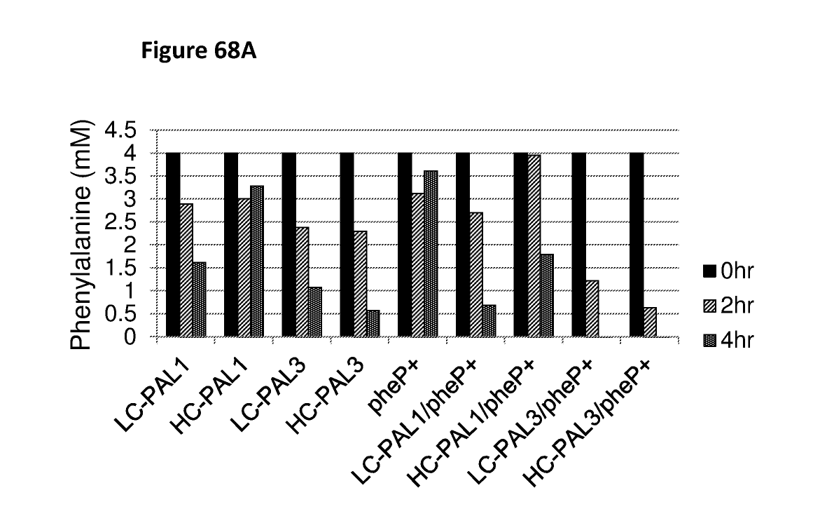

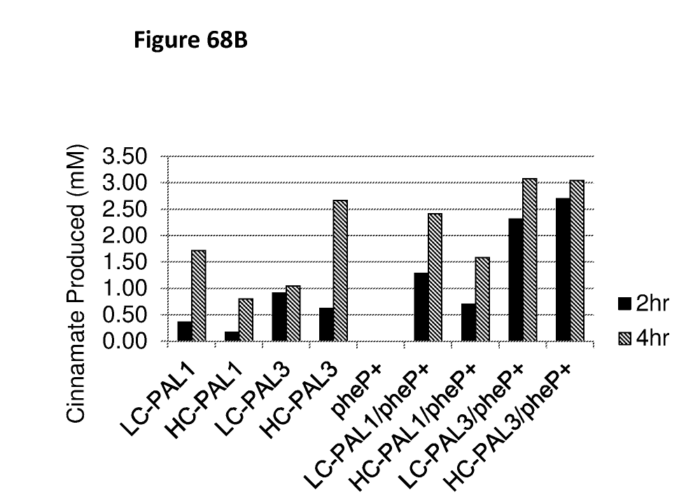

For in vitro studies, all incubations were performed at 37° C. Cultures of E. coli Nissle transformed with a plasmid comprising the PAL gene driven by the Tet promoter were grown overnight and then diluted 1:100 in LB. The cells were grown with shaking (200 rpm) to early log phase. Anhydrous tetracycline (ATC) was added to cultures at a concentration of 100 ng/mL to induce expression of PAL, and bacteria were grown for another 2 hrs. Bacteria were then pelleted, washed, and resuspended in minimal media, and supplemented with 4 mM phenylalanine. Aliquots were removed at 0 hrs, 2 hrs, and 4 hrs for phenylalanine quantification (FIG. 68A), and at 2 hrs and 4 hrs for cinnamate quantification (FIG. 68B), by mass spectrometry, as described in Examples 24-26. As shown in FIG. 68, expression of pheP in conjunction with PAL significantly enhances the degradation of phenylalanine as compared to PAL alone or pheP alone. Notably, the additional copy of pheP permitted the complete degradation of phenylalanine (4 mM) in 4 hrs (FIG. 68A). FIG. 68B depicts cinnamate levels in samples at 2 hrs and 4 hrs post-induction. Since cinnamate production is directly correlated with phenylalanine degradation, these data suggest that phenylalanine disappearance is due to phenylalanine catabolism, and that cinnamate may be used as an alternative biomarker for strain activity. PheP overexpression improves phenylalanine metabolism in engineered bacteria.

In conclusion, in conjunction with pheP, even low-copy PAL-expressing plasmids are capable of almost completely eliminating phenylalanine from a test sample (FIGS. 68A and 68B). Furthermore, without wishing to be bound by theory, in some embodiments, that incorporate pheP, there may be additional advantages to using a low-copy PAL-expressing plasmid in conjunction with pheP in order to enhance the stability of PAL expression while maintaining high phenylalanine metabolism, and to reduce negative selection pressure on the transformed bacterium. In alternate embodiments, the phenylalanine transporter is used in conjunction with a high-copy PAL-expressing plasmid.

### Example 29. Production of PAL from FNR Promoter in Recombinant E. coli

Cultures of E. coli Nissle transformed with a plasmid comprising the PAL gene driven by any of the exemplary FNR promoters were grown overnight and then diluted 1:200 in LB. The bacterial cells may further comprise the pheP gene driven by the Tet promoter and incorporated into the chromosome. ATC was added to cultures at a concentration of 100 ng/mL to induce expression of pheP, and the cells were grown with shaking at 250 rpm either aerobically or anaerobically in a Coy anaerobic chamber supplied with 90% N₂, 5% CO₂, and 5% H₂. After 4 hrs of incubation, cells were pelleted down, washed, and resuspended in M9 minimal medium supplemented with 0.5% glucose and 4 mM phenylalanine. Aliquots were collected at 0 hrs, 2 hrs, 4 hrs, and 24 hrs for phenylalanine quantification. The genetically engineered bacteria expressing PAL3 driven by the FNR promoter are more efficient at removing phenylalanine from culture medium under anaerobic conditions, compared to aerobic conditions. The expression of pheP in conjunction with PAL3 further decreased levels of phenylalanine.

### Example 30. Phenylalanine Degradation in Recombinant E. coli with and without pheP Overexpression

The SYN-PKU304 and SYN-PKU305 strains contain low-copy plasmids harboring the PAL3 gene, and a copy of pheP integrated at the lacZ locus. The SYN-PKU308 and SYN-PKU307 strains also contain low-copy plasmids harboring the PAL3 gene, but lack a copy of pheP integrated at the lacZ locus. In all four strains, expression of PAL3 and pheP (when applicable) is controlled by an oxygen level-dependent promoter.

To determine rates of phenylalanine degradation in engineered E. coli Nissle with and without pheP on the chromosome, overnight cultures of SYN-PKU304 and SYN-PKU307 were diluted 1:100 in LB containing ampicillin, and overnight cultures of SYN-PKU308 and SYN-PKU305 were diluted 1:100 in LB containing kanamycin. All strains were grown for 1.5 hrs before cultures were placed in a Coy anaerobic chamber supplying 90% N₂, 5% CO₂, and 5% H₂. After 4 hrs of induction, bacteria were pelleted, washed in PBS, and resuspended in 1 mL of assay buffer. Assay buffer contained M9 minimal media supplemented with 0.5% glucose, 8.4% sodium bicarbonate, and 4 mM of phenylalanine.

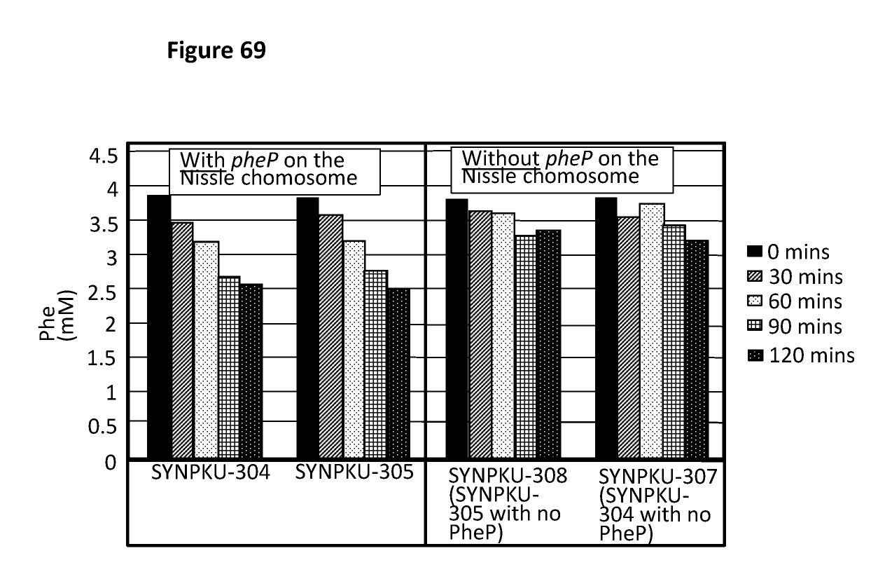

For the activity assay, starting counts of colony-forming units (cfu) were quantified using serial dilution and plating. Aliquots were removed from each cell assay every 30 min for 3 hrs for phenylalanine quantification by mass spectrometry. Specifically, 150 μL of bacterial cells were pelleted and the supernatant was harvested for LC-MS analysis, with assay media without cells used as the zero-time point. FIG. 69 shows the observed phenylalanine degradation for strains with pheP on the chromosome (SYN-PKU304 and SYN-PKU305; left), as well as strains lacking pheP on the chromosome (SYN-PKU308 and SYN-PKU307; right). These data show that pheP overexpression is important in order to increase rates of phenylalanine degradation in synthetic probiotics.

### Example 31. Activity of Strains with Single and Multiple Chromosomal PAL3 Insertions

To assess the effect of insertion site and number of insertions on the activity of the genetically engineered bacteria, in vitro activity of strains with different single insertions of PAL3 at various chromosomal locations and with multiple PAL3 insertions was measured.

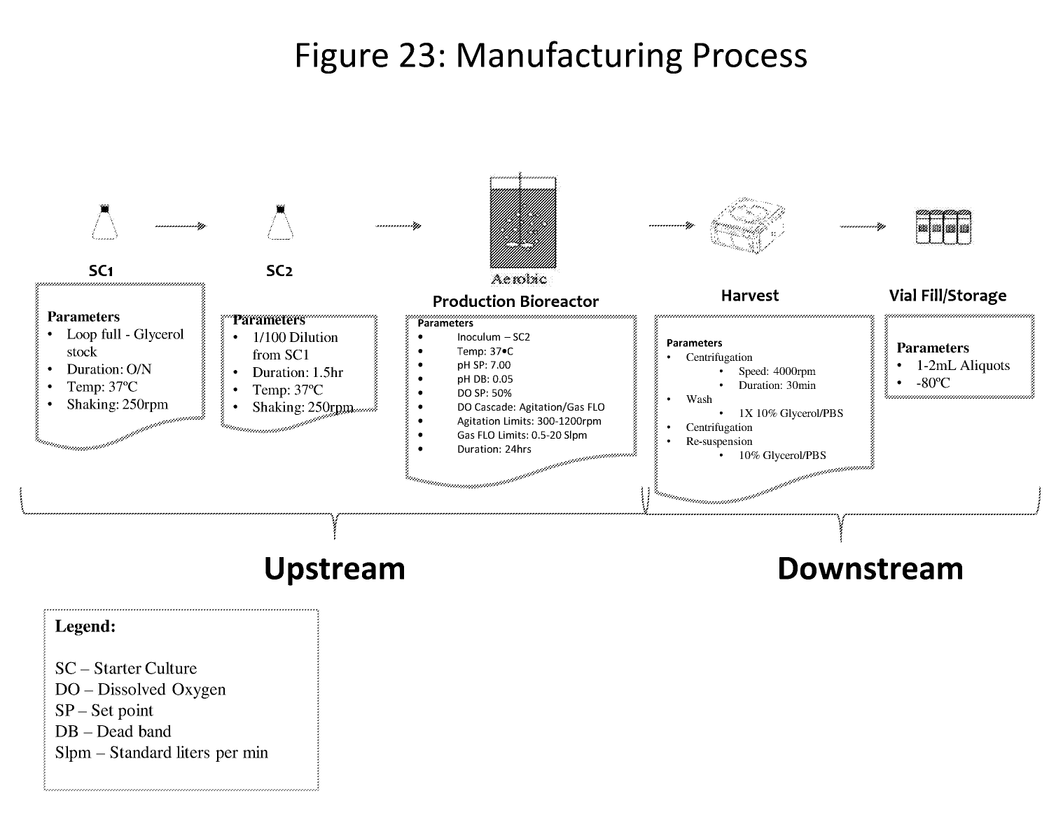

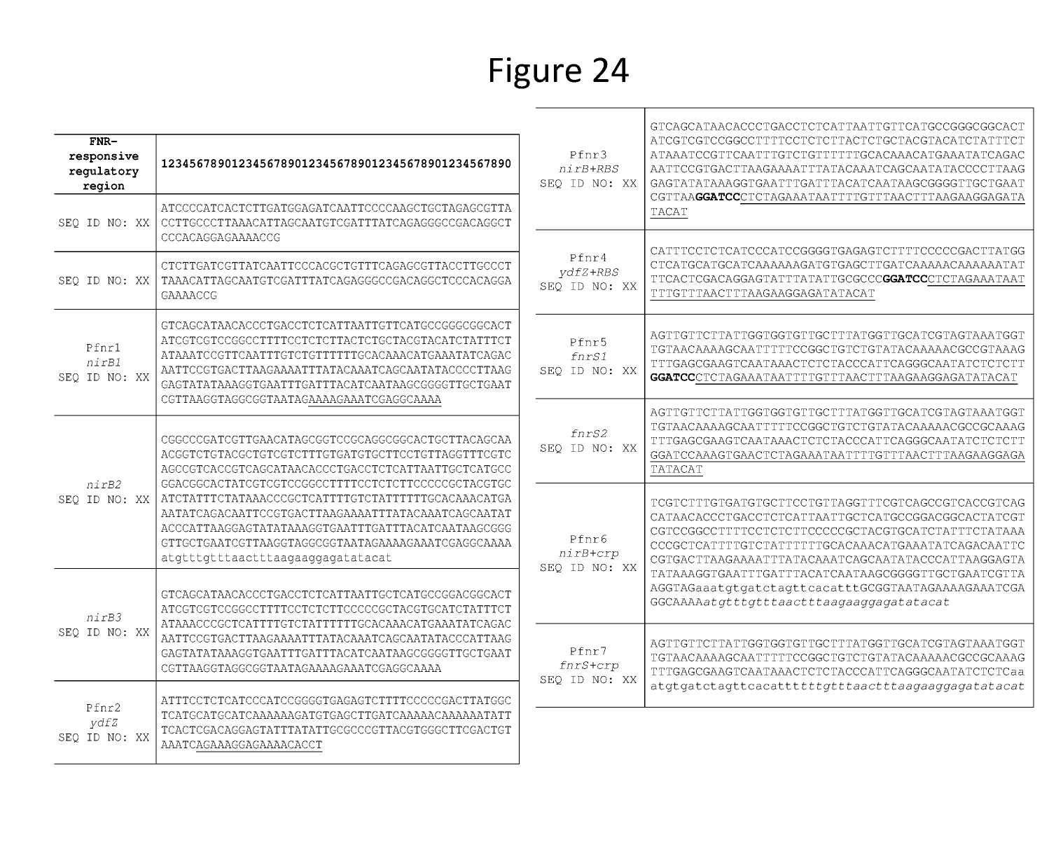

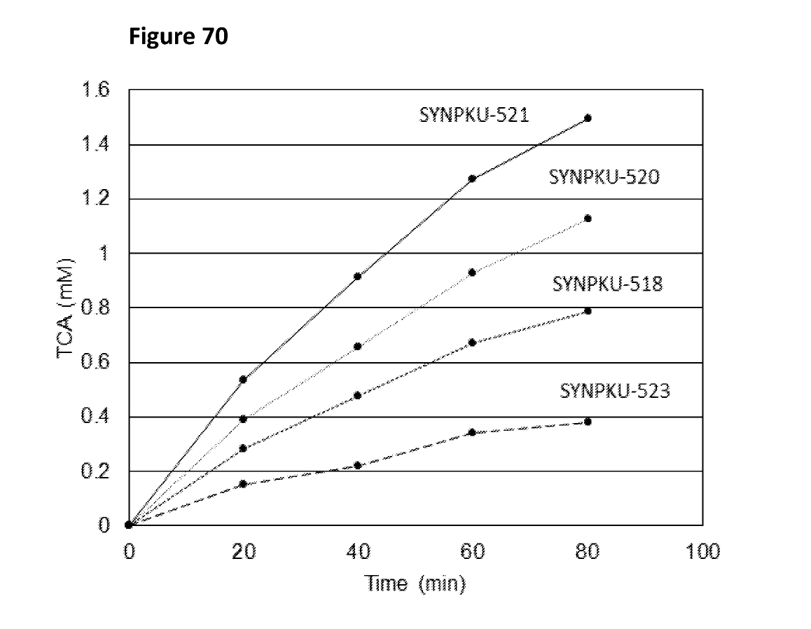

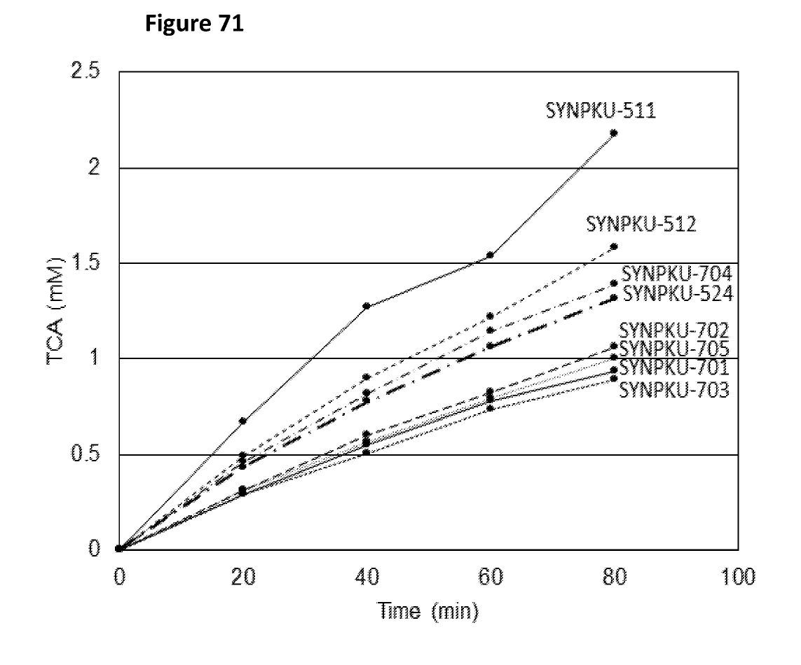

Cells were grown overnight in LB and diluted 1:100. After 1.5 hrs of growth, cultures were placed in Coy anaerobic chamber supplying 90% N₂, 5% CO₂, and 5% H₂. After 4 hrs of induction, bacteria were resuspended in assay buffer containing 50 mM phenylalanine. Aliquots were removed from cell assays every 20 min for 1.5 hrs for trans-cinnamate quantification by absorbance at 290 nm. Results are shown in FIGS. 70 and 71 and Tables below. FIG. 70 depicts trans-cinnamate concentrations (PAL activity) for strains comprising single PAL3 insertions at various locations on the chromosome. FIG. 23 depicts trans-cinnamate concentrations (PAL activity) for strains comprising multiple PAL3 insertions at various locations on the chromosome.

Activity of Various Strains Comprising a Single PAL3 Chromosomal Insertion at Various Sites

| | | rate |
|---|---|---|
| | | (umol/hr./1e9 |
| Insertion: | Strain: | cells): |
| agaI/rsmI | SYN- | 1.97 |
| | PKU520 | |
| yicS/nepI | SYN- | 2.44 |
| | PKU521 | |
| cea | SYN- | ND |
| | PKU522 | |
| malEK | SYN- | 1.66 |
| | PKU518 | |
| malPT | SYN- | 0.47 |
| | PKU523 | |

In Vitro Activity of Various Strains Comprising One or More Chromosomal PAL3 Insertions

| | | Rate |
|---|---|---|
| | | (umol/ |
| | | hr./1e9 |
| Genotypes: | Strain | cells) |
| agaI:PAL, cea:PAL, matPT:PAL, malEK:PAL, | SYN- | 6.76 |
| lacZ:pheP, thyA- | PKU512 | |
| agaI:PAL, yicS:PAL, cea:PAL, matPT:PAL, | SYN- | 7.65 |
| malEK:PAL, lacZ:pheP, thyA- | PKU511 | |
| malPT:PAL, malEK:PAL, lacZ:pheP | SYN- | 2.89 |
| | PKU524 | |
| malEK:PAL, lacZ:pheP, ara-LAAD | SYN- | 1.53 |
| | PKU702 | |
| malPT:PAL, malEK:PAL, lacZ:pheP, ara-LAAD | SYN- | 2.65 |
| | PKU701 | |
| malPT:PAL, malEK:PAL, lacZ:pheP, agaI:pheP, | SYN- | 3.14 |
| ara-LAAD | PKU703 | |
| yicS:PAL, malPT:PAL, malEK:PAL lacZ:pheP, ara- | SYN- | 3.47 |
| LAAD | PKU704 | |
| yicS:PAL, malPT:PAL, malEK:PAL, lacZ:pheP, | SYN- | 3.74 |
| agaI:pheP, ara-LAAD | PKU705 | |

### Example 32. Activity of a Strain with Five Chromosomal Copies of PAL3

The activity of a strain SYN-PKU511, a strain comprising five integrated copies of an anaerobically (FNR) controlled PAL3 and an anaerobically controlled pheP integrated in the lacZ locus, was assessed.

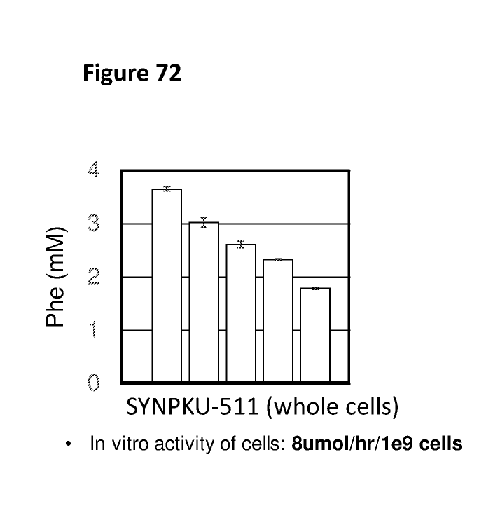

The genetically engineered bacteria were grown overnight, diluted and allowed to grow for another 2.5 hours. Cultures were then placed in Coy anaerobic chamber supplying 90% N2, 5% CO2, and 5% H2. After 3.5 hrs of induction in phenylalanine containing medium (4 mM phenylalanine), whole cell extracts were prepared every 30 min for 3 hrs and phenylalanine was quantified by mass spectrometry. Results are shown in FIG. 72. The in vitro activity of the cells was 8 umol/hr./1e9 cells. Phenylalanine levels drop to about half of the original levels after 2 hours.

### Example 33. Activity of a Strain Expressing LAAD

To assess whether LAAD expression can be used as an alternative, additional or complementary phenylalanine degradation means to PAL3, the ability of genetically engineered strain SYN-PKU401, which contains a high copy plasmid expressing LAAD driven by a Tet-inducible promoter, was measured at various cell concentrations and at varying oxygen levels.

Overnight cultures of SYN-PKU401 were diluted 1:100 and grown to early log phase before induction with ATC (100 ng/ml) for 2 hours. Cells were spun down and incubated as follows.

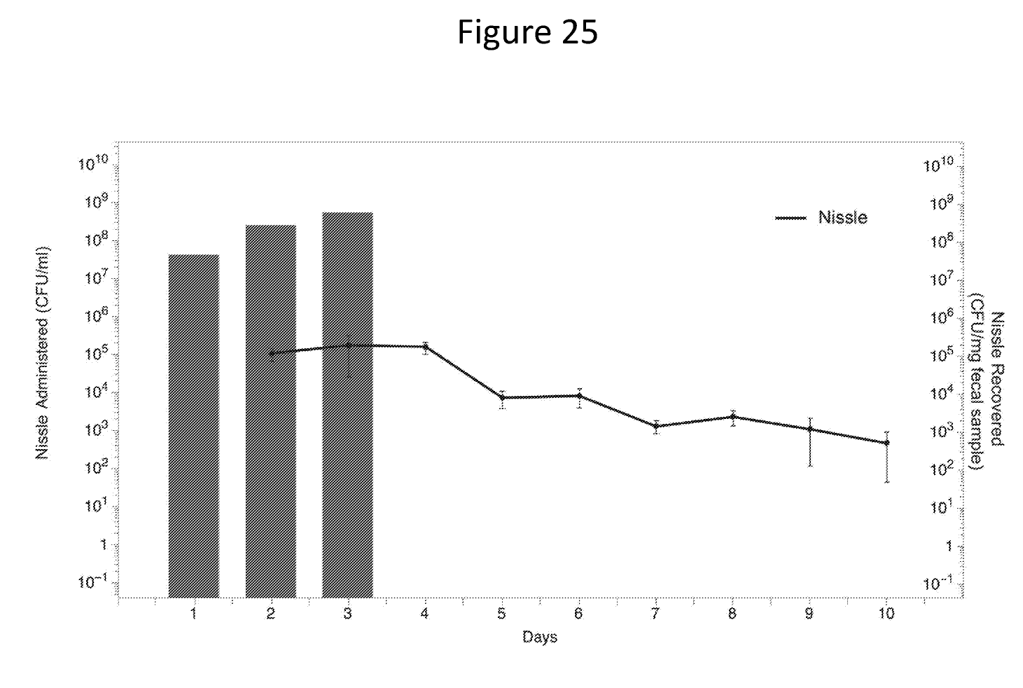

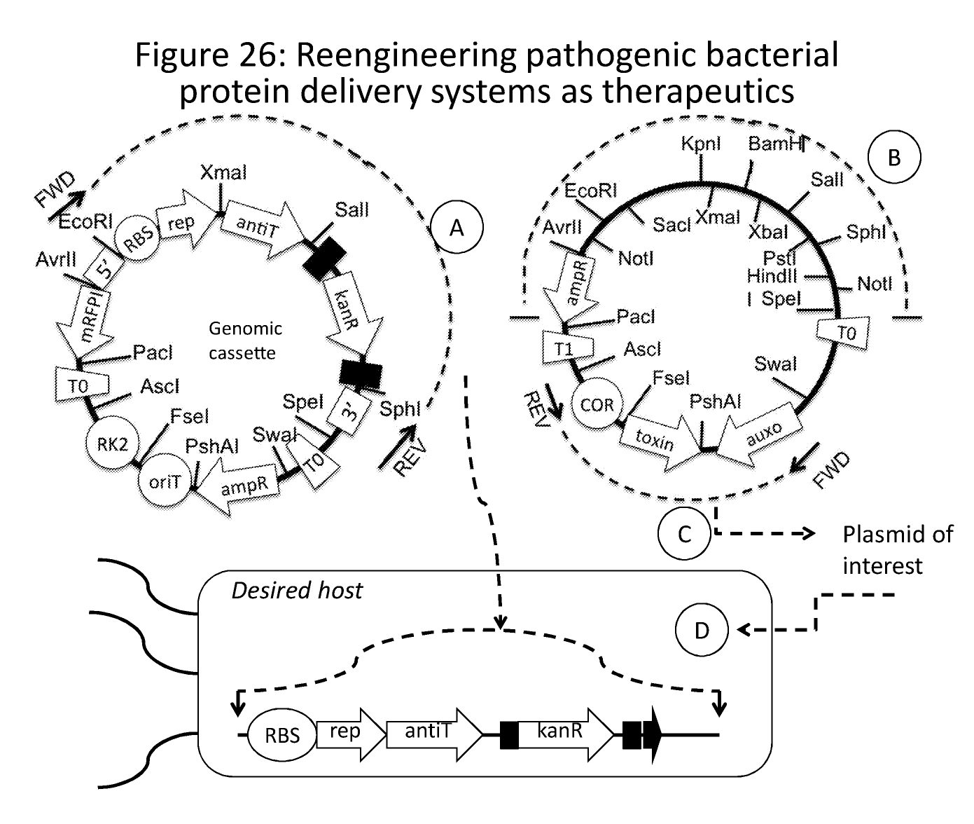

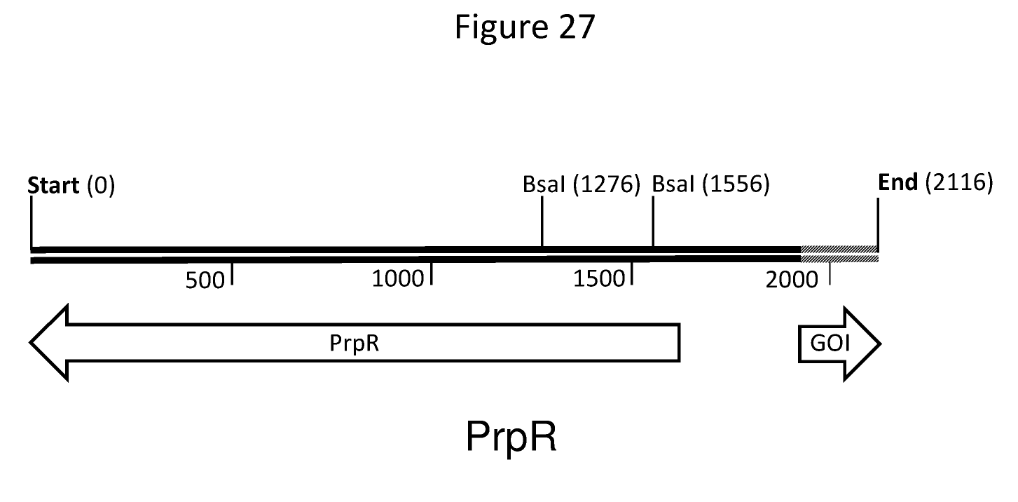

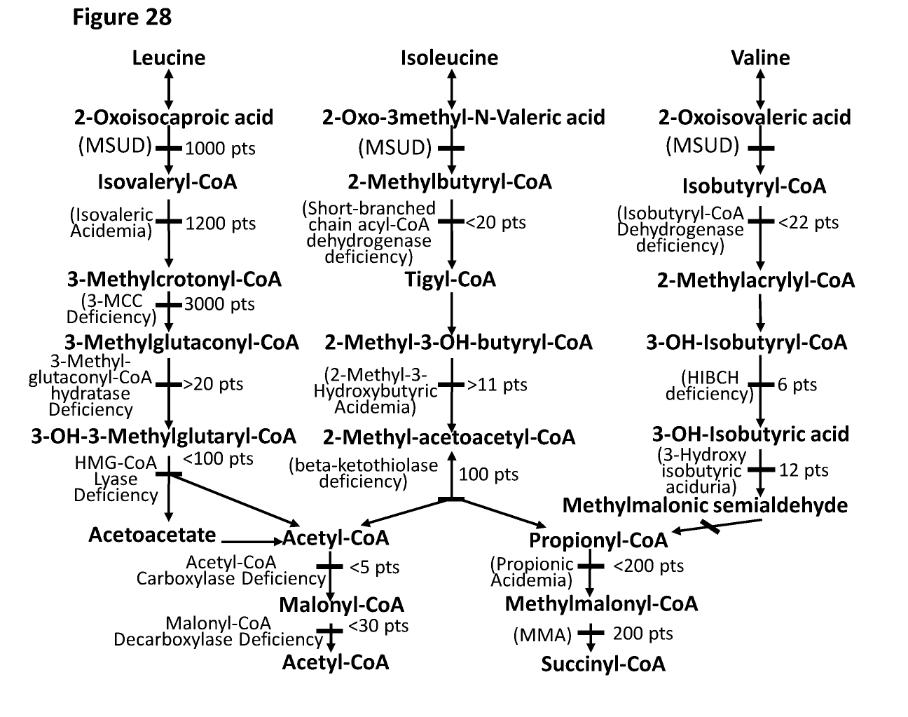

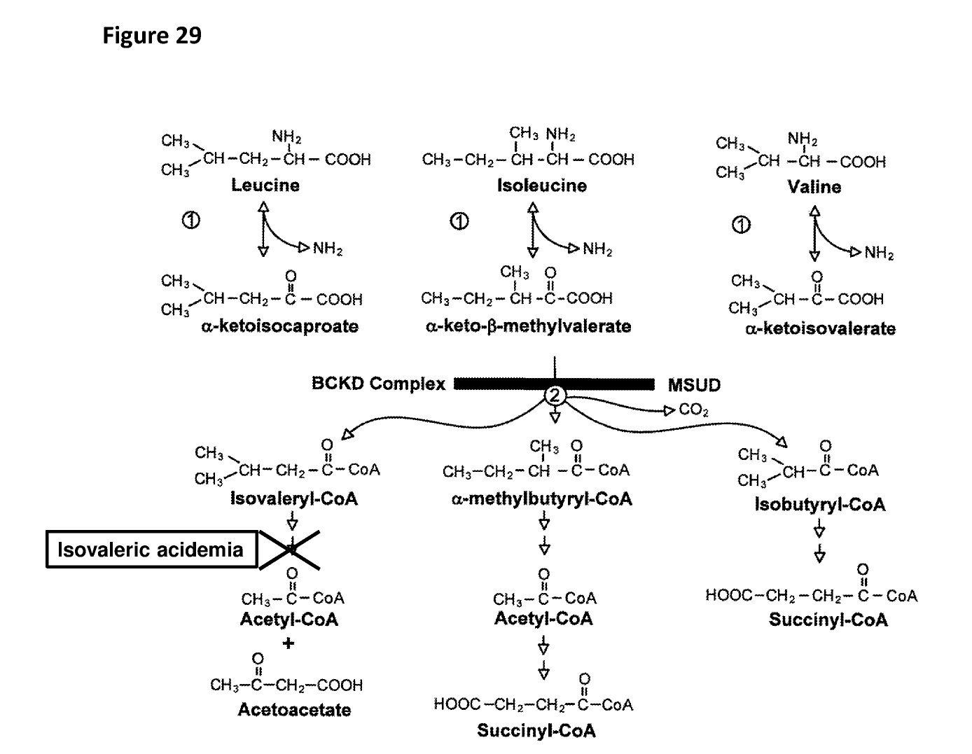

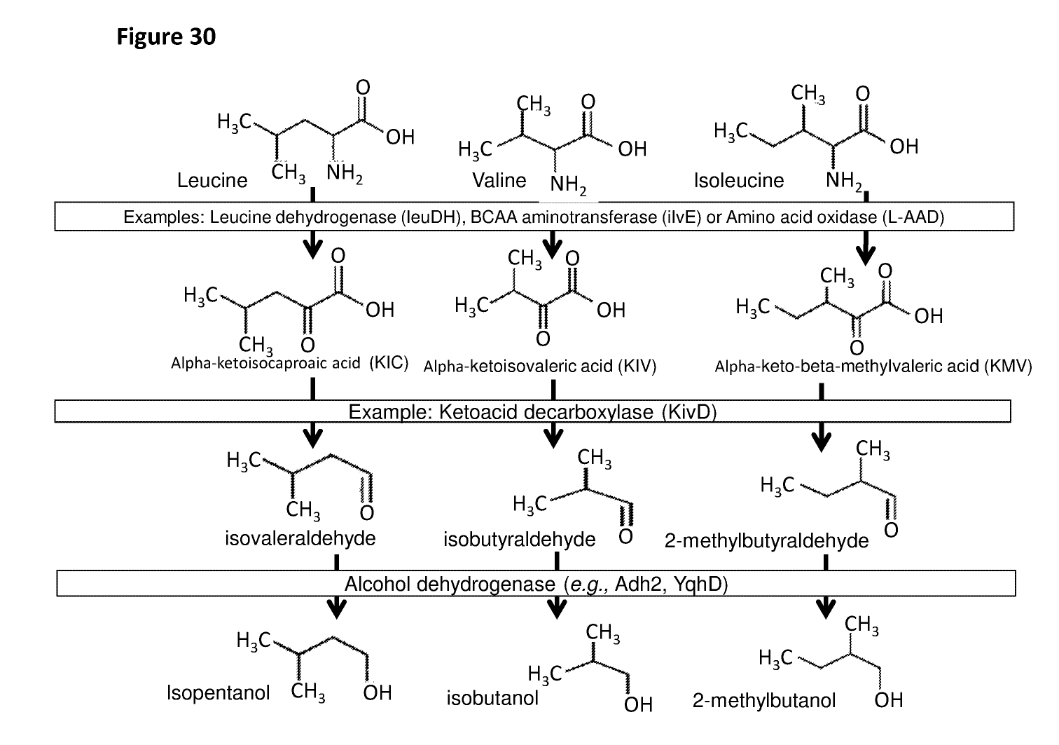

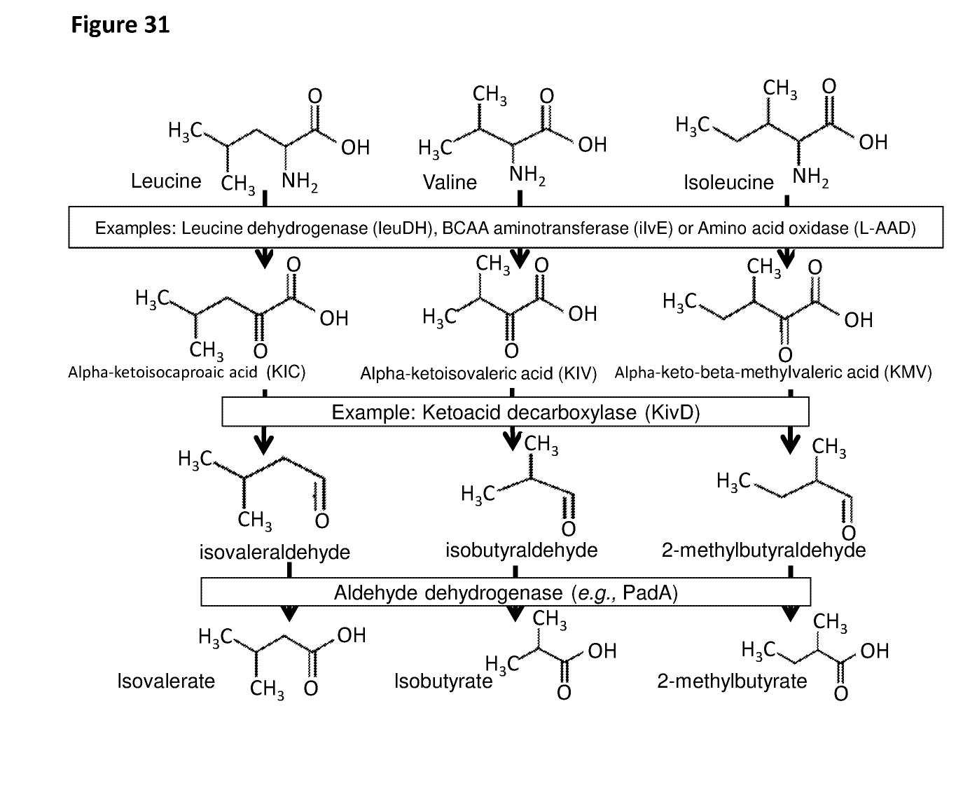

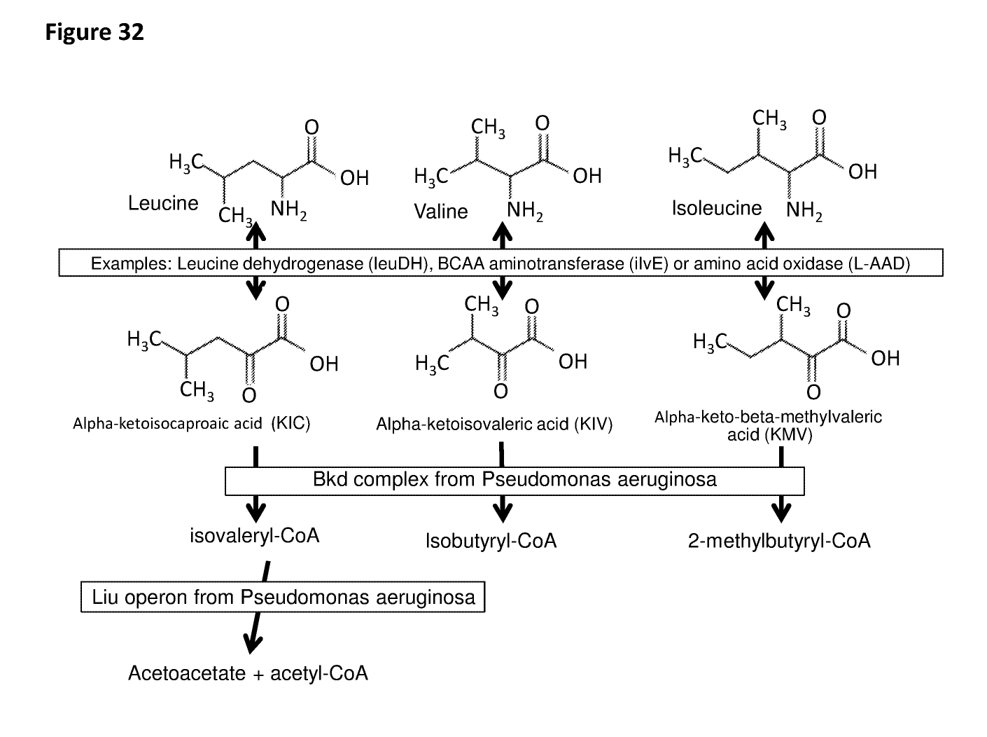

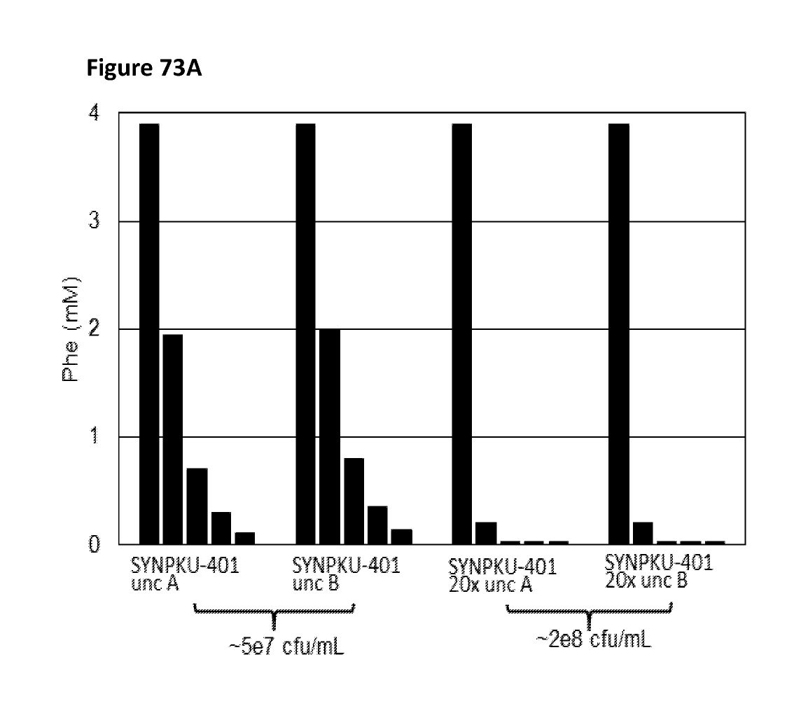

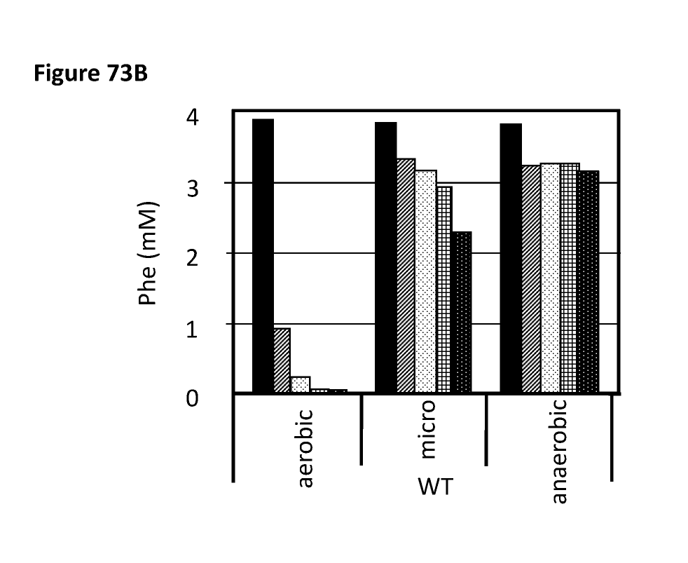

Cells (1 ml) were incubated aerobically in a 14 ml culture tube, shaking at 250 rpm (FIGS. 25 A and B). For microaerobic conditions, cells (1 ml) were incubated in a 1.7 ml conical tube without shaking. Cells were incubated anaerobically in a Coy anaerobic chamber supplying 90% N2, 5% CO2, and 5% H2 (FIG. 25B). Aliquots were removed from cell assays every 30 min for 2 hrs for phenylalanine quantification by mass spectrometry, and results are shown in FIGS. 73A and 73B. FIG. 73A shows cell concentration dependent aerobic activity. The activity in aerobic conditions is ˜50 umol/hr./1e9 cells, and some activity is retained under microaerobic conditions, which may allow for activity in environments with oxygen concentrations less than ambient air. The activity of SYN-PKU401 under microaerobic conditions is comparable to SYN-PKU304 under anaerobic conditions, however, activity seems to be dependent on cell density.

### Example 34. Efficacy of PAL-Expressing Bacteria in a Mouse Model of PKU

For in vivo studies, BTBR-Pah^{enu2} mice were obtained from Jackson Laboratory and bred to homozygosity for use as a model of PKU. Bacteria harboring a low-copy pSC101 origin plasmid expressing PAL3 from the Tet promoter, as well as a copy of pheP driven by the Tet promoter integrated into the genome (SYN-PKU302), were grown. SYN-PKU1 was induced by ATC for 2 hrs prior to administration. Bacteria were resuspended in phosphate buffered saline (PBS) and 10⁹ ATC-induced SYN-PKU302 or control Nissle bacteria were administered to mice by oral gavage.

At the beginning of the study, mice were given water that was supplemented with 100 micrograms/mL ATC and 5% sucrose. Mice were fasted by removing chow overnight (10 hrs), and blood samples were collected by mandibular bleeding the next morning in order to determine baseline phenylalanine levels. Blood samples were collected in heparinized tubes and spun at 2G for 20 min to produce plasma, which was then removed and stored at −80° C. Mice were given chow again, and were gavaged after 1 hr. with 100 μL (5×10⁹ CFU) of bacteria that had previously been induced for 2 hrs with ATC. Mice were put back on chow for 2 hrs. Plasma samples were prepared as described above.

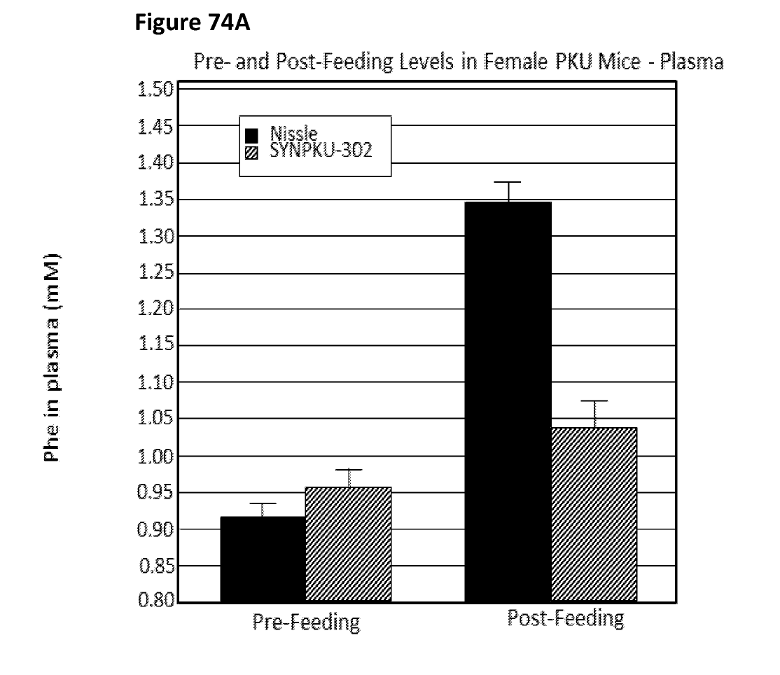

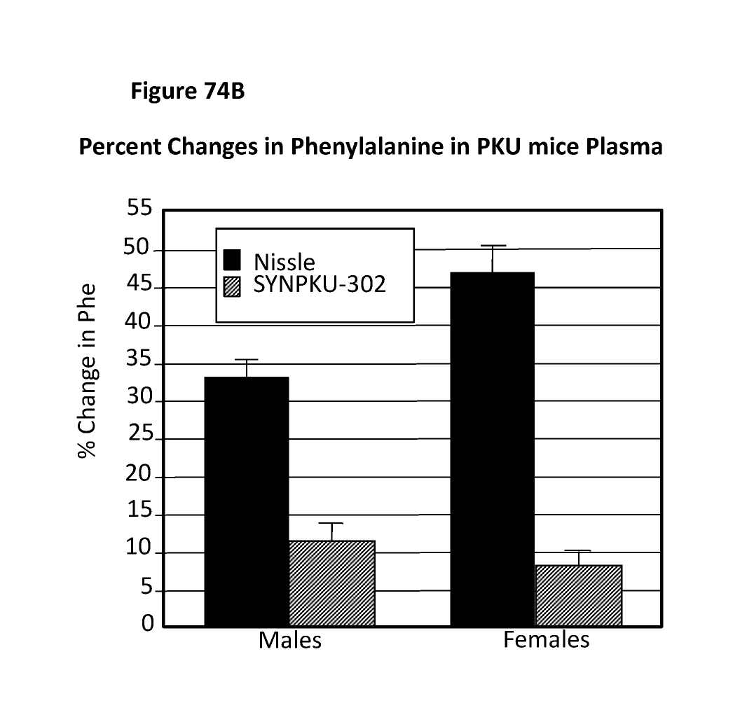

FIG. 74A shows phenylalanine levels before and after feeding, and FIG. 74B shows the percent (%) change in blood phenylalanine levels before and after feeding as a male or female group average (p<0.01). As shown in FIG. 74, PKU mice treated with SYN-PKU1 exhibit a significantly reduced post-feeding rise in serum phenylalanine levels compared to controls.

### Example 35. Efficacy of PAL-Expressing Bacteria Following Subcutaneous Phenylalanine Challenge

Streptomycin-resistant E. coli Nissle (SYN-PKU901) was grown from frozen stocks to a density of 10¹⁰ cells/mL. Bacteria containing a copy of pheP under the control of a Tet promoter integrated into the lacZ locus, as well as a high-copy plasmid expressing PAL3 under the control of a Tet promoter (SYN-PKU303) were grown to an A₆₀₀ of 0.25 and then induced by ATC (100 ng/mL) for 4 hrs. Bacteria were centrifuged, washed, and resuspended in bicarbonate buffer at density of 1×10¹⁰ cells/mL before freezing at −80° C.

Beginning at least 3 days prior to the study (i.e., Days −6 to −3), homozygous BTBR-Pah^{enu2} mice (approx. 6-12 weeks of age) were maintained on phenylalanine-free chow and water that was supplemented with 0.5 grams/L phenylalanine. On Day 1, mice were randomized into treatment groups and blood samples were collected by sub-mandibular skin puncture to determine baseline phenylalanine levels. Mice were also weighed to determine the average weight for each group. Mice were then administered single dose of phenylalanine by subcutaneous injection at 0.1 mg per gram body weight, according to the average group weight. At 30 and 90 min post-injection, 200 μL of H₂O (n=30), SYN-PKU901 (n=33), or SYN-PKU303 (n=34) were administered to mice by oral gavage. Blood samples were collected at 2 hrs and 4 hrs following phenylalanine challenge, and phenylalanine levels in the blood were measured using mass spectrometry.

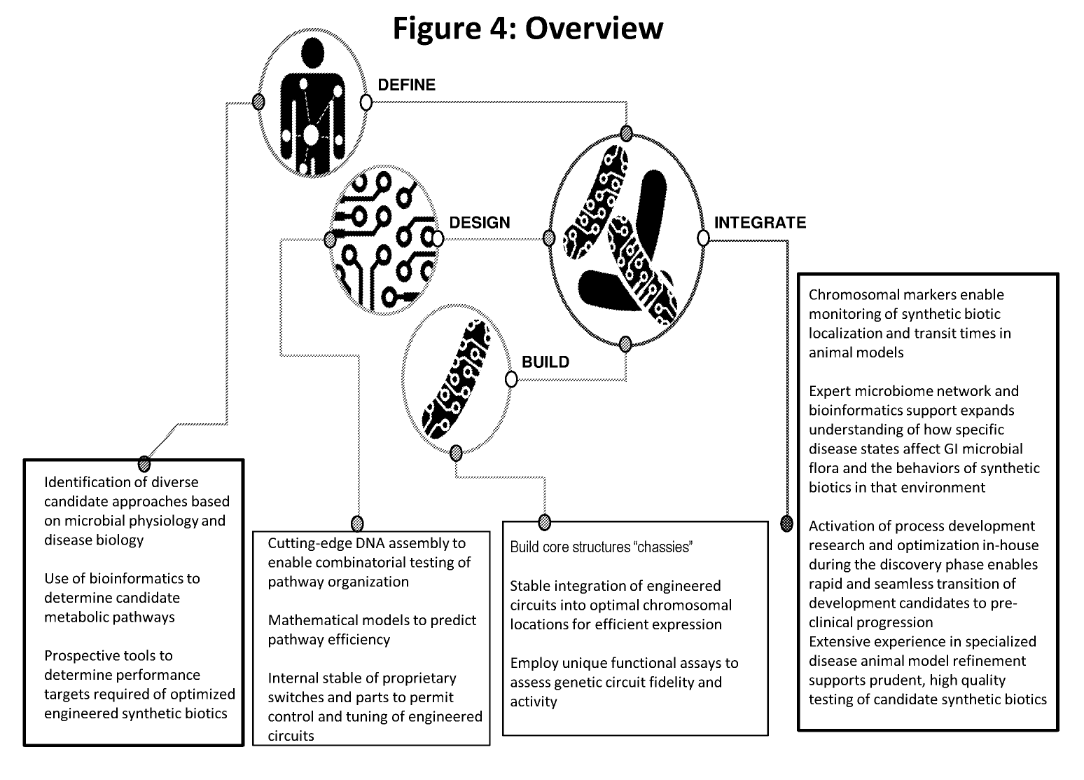

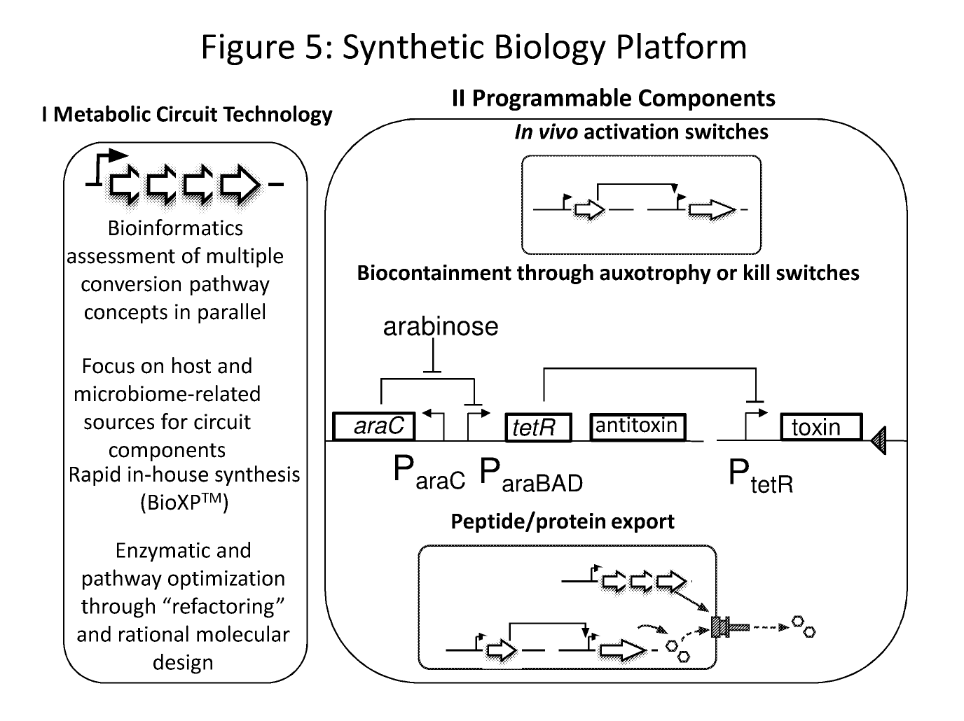

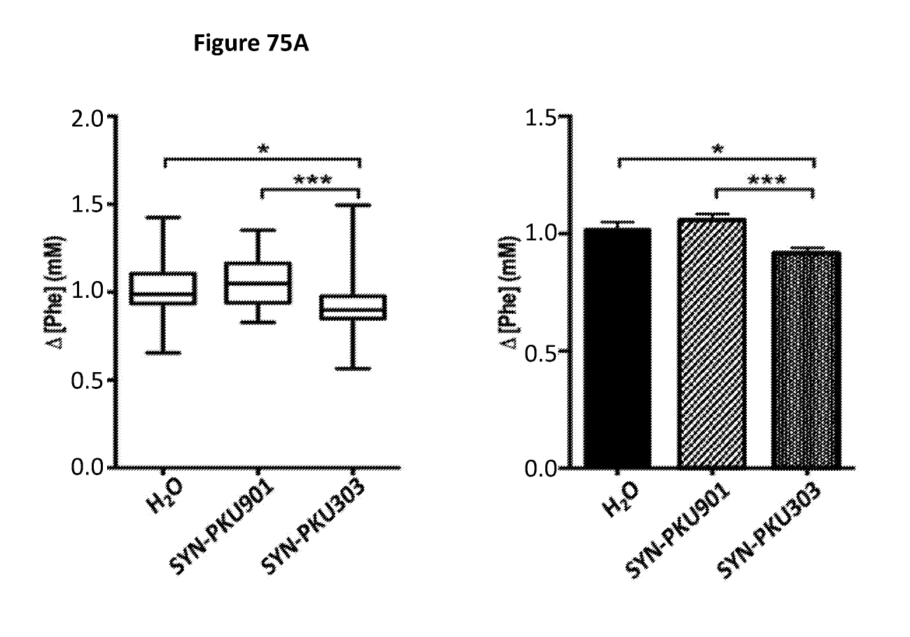

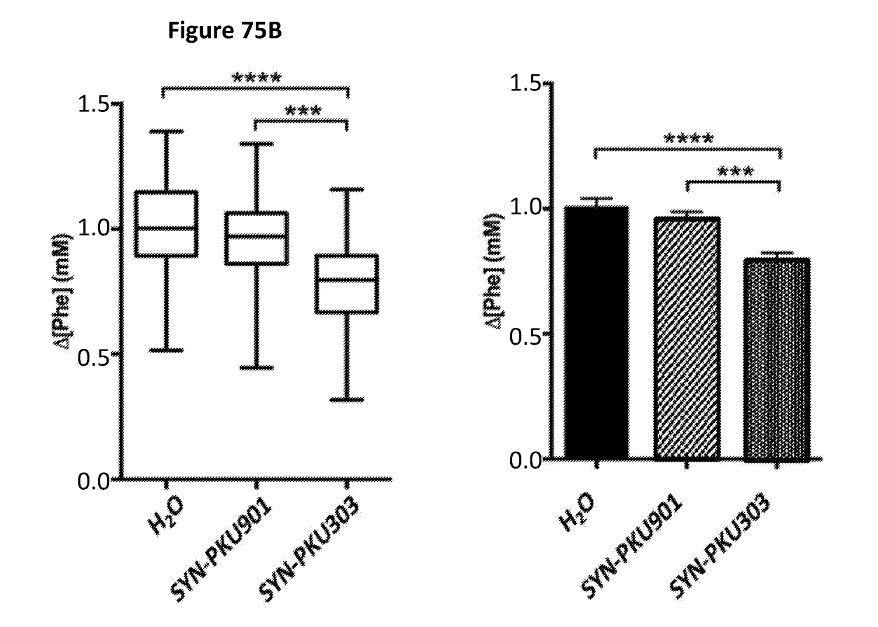

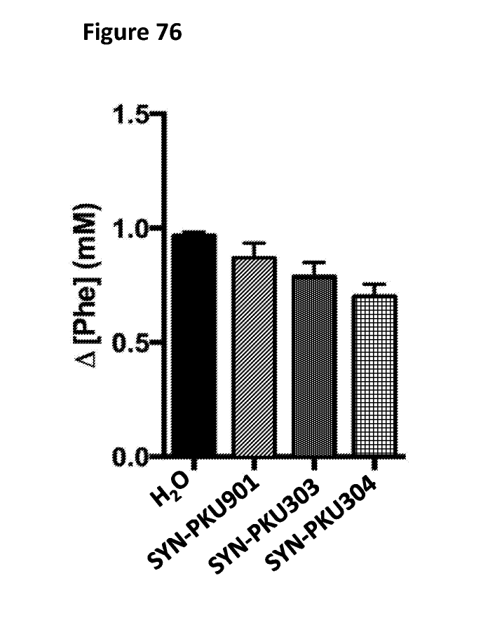

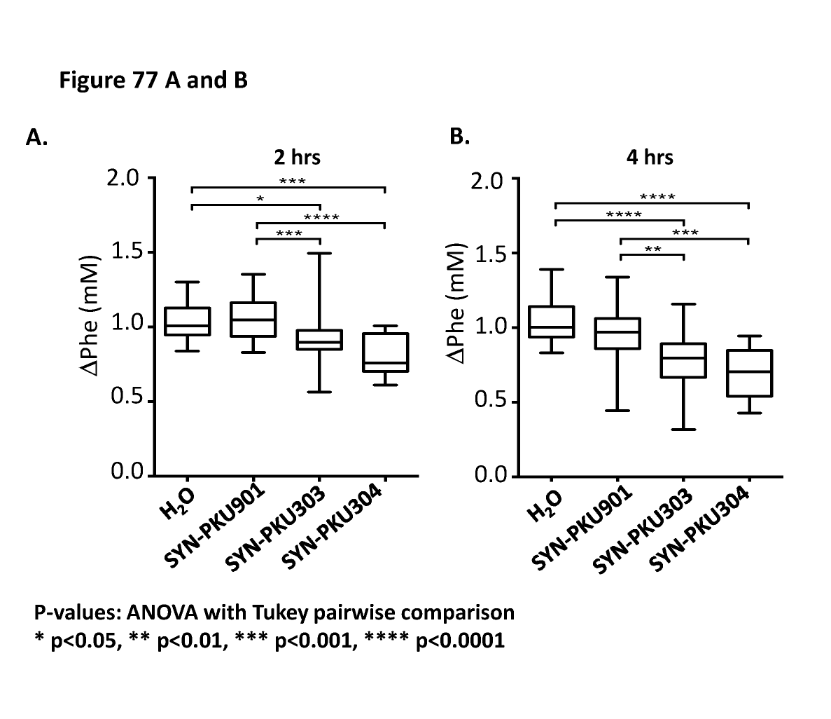

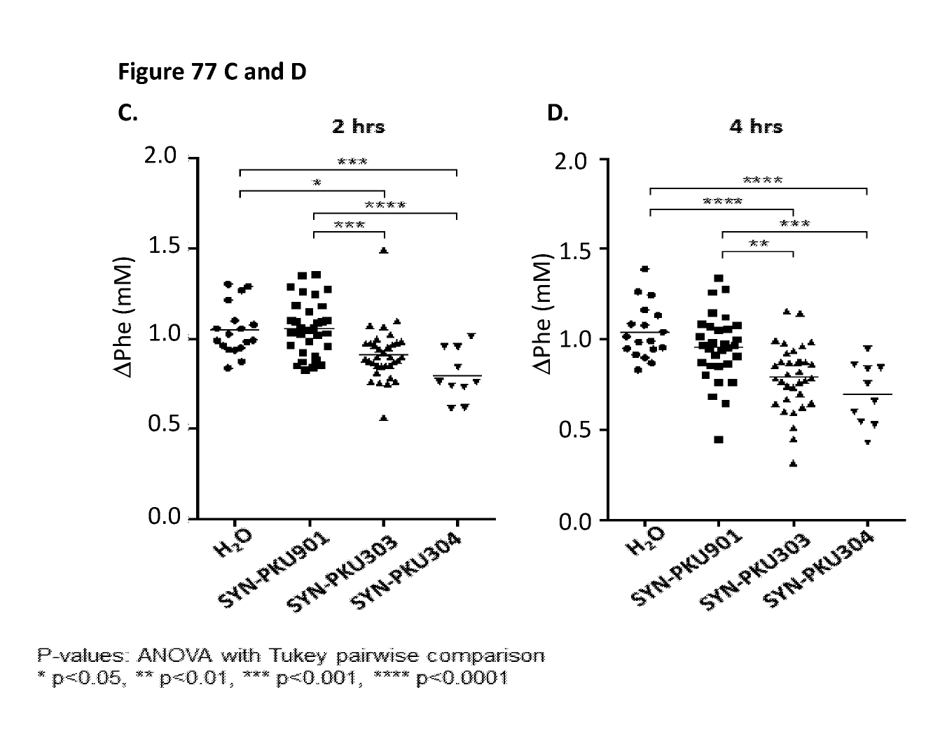

FIG. 75 shows phenylalanine blood concentrations relative to baseline concentrations at 2 hrs (FIG. 75A) and 4 hrs (FIG. 75B) post-phenylalanine injection. These data suggest that subcutaneous injection of phenylalanine causes hyperphenylalanemia in homozygous enu2/enu2 mice, and that oral administration of SYN-PKU303 significantly reduces blood phenylalanine levels following phenylalanine challenge, compared to control groups (p<0.00001 at 4 hrs). Moreover, these results confirm that the orally-administered engineered bacteria, and not the non-engineered Nissle parent, can significantly impact blood-phenylalanine levels independent of dietary exposure. Thus, a PKU-specific probiotic may not need to be co-administered in conjunction with diet.

### Example 39. Dose-Response Activity of PAL-Expressing Bacteria on Systemic Phenylalanine

Streptomycin-resistant E. coli Nissle (SYN-PKU901) were grown from frozen stocks to a density of 10¹⁰ cells/mL. Bacteria containing a copy of pheP under the control of a P_{fnrS} promoter integrated into the lacZ locus, as well as a low-copy plasmid expressing PAL3 under the control of a P_{fnrS} promoter (SYN-PKU304) were grown to an A₆₀₀ of 0.25 and then induced anaerobically by purging the bacterial fermenter with nitrogen for 4 hrs. Bacteria were centrifuged, washed, and resuspended in bicarbonate buffer at density of 5×10⁹ cells/mL before freezing at −80° C.

Beginning at least 3 days prior to the study (i.e., Days −6 to −3), mice were maintained on phenylalanine-free chow and water that was supplemented with 0.5 grams/L phenylalanine. On Day 1, mice were randomized into treatment groups and blood samples were collected by sub-mandibular skin puncture to determine baseline phenylalanine levels. Mice were also weighed to determine the average weight for each group. Mice were then administered single dose of phenylalanine by subcutaneous injection at 0.1 mg per gram body weight, according to the average group weight. At 30 and 90 min post-injection, 200 μL of H₂O (n=12), 200 μL of SYN-PKU901 (n=12), or 100 μL, 200 μL, or 400 μL of SYN-PKU304 (n=12 in each dose group) were administered to mice by oral gavage. Blood samples were collected at 2 hrs and 4 hrs following phenylalanine challenge, and phenylalanine levels in the blood were measured using mass spectrometry.

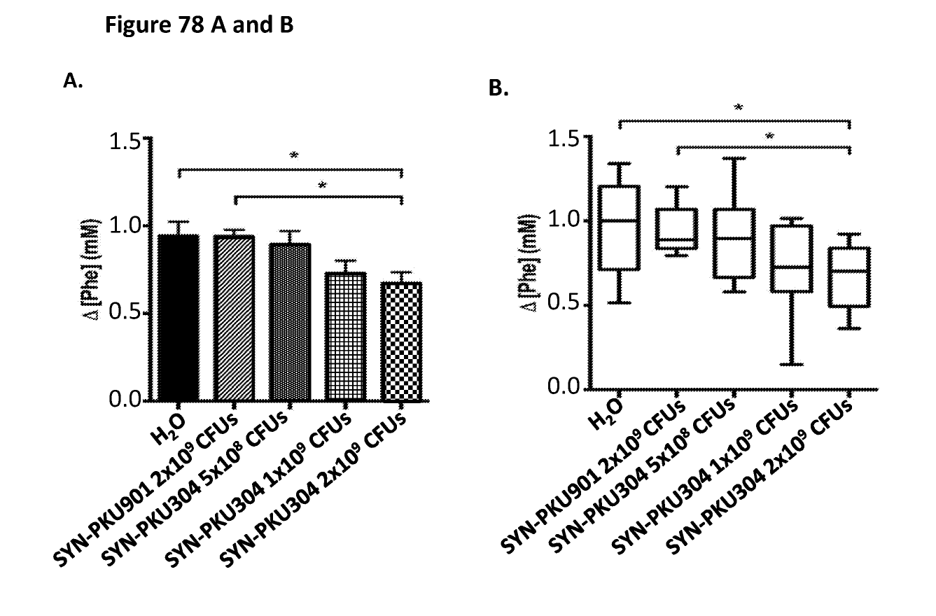

FIG. 78 shows phenylalanine blood concentrations relative to baseline concentrations post-phenylalanine injection. These data demonstrate a dose-dependent decrease in blood phenylalanine levels in SYN-PKU304-treated mice compared to mock treatment (H₂O) or administration of the parental strain (SYN-PKU901), following subcutaneous injection of phenylalanine (* 30% decrease; p<0.05).

### Example 40. Phenylalanine Degradation Activity In Vivo (PAL)

To compare the correlation between in vivo and in vitro phenylalanine activity, SYN-PKU304 (containing a low copy plasmin expressing PAL3 with a chromosomal insertion of PfnrS-pheP at the LacZ locus, was compared to SYN-PKU901, a control Nissle strain with streptomycin resistance in vivo).

Beginning at least 3 days prior to the study (i.e., Days −6 to −3), homozygous BTBR-Pah^{enu2} mice (approx. 6-12 weeks of age) were maintained on phenylalanine-free chow and water that was supplemented with 0.5 grams/L phenylalanine. On Day 1, mice were randomized into treatment groups and blood samples were collected by sub-mandibular skin puncture to determine baseline phenylalanine levels. Mice were also weighed to determine the average weight for each group. Mice were then administered single dose of phenylalanine by subcutaneous injection at 0.1 mg per gram body weight, according to the average group weight. At 30 and 90 min post-injection, the bacteria were administered to mice by oral gavage.

To prepare the cells, cells were diluted 1:100 in LB (2 L), grown for 1.5 h aerobically, then shifted to the anaerobe chamber for 4 hours. Prior to administration, cells were concentrated 200× and frozen (15% glycerol, 2 g/L glucose, in PBS). Cells were thawed on ice, and 4e10 cfu/mL and mixed 9:1 in 1M bicarbonate. Each mouse gavaged 800 uL total, or 2.9e10 cfu/mouse.

Blood samples were collected at 2 hrs and 4 hrs following phenylalanine challenge, and phenylalanine levels in the blood were measured using mass spectrometry, and the change in Phenylalanine concentration per hour was calculated. The total metabolic activity measured was 81.2 umol/hr. and the total reduction in change in phenylalanine was 45% (P<0.05). These same cells showed an in vitro activity of 2.8 umol/hr./1e9 cells.

Additionally, various metabolites were measured to determine whether secondary metabolites can be used as an additional parameter to assess the rate of phenylalanine consumption of the engineered bacteria. When PAH activity is reduced in PKU, the accumulated phenylalanine is converted into PKU specific metabolites phenylpyruvate, which can be further converted into phenyllactic acid. In the presence of the genetically engineered bacteria, phenylalanine is converted by PAL to PAL specific metabolites trans-cinnamic acid, which then can be further converted by liver enzymes to hippuric acid (data not shown). Blood samples were analyzed for phenylpyruvate, phenyllactate, trans-cinnamic acid, and hippuric acid as described in Example 24-26. Results are consistent with the phenylalanine degradation. For SYN-PKU304, PAL specific metabolites are detected at 4 hours, and moreover, lower levels of PKU specific metabolites are observed as compared to SYN-PKU901, indicating that PAL phenylalanine degradation may cause a shift away from PKU specific metabolites in favor or PAL specific metabolites.

### Example 41. Phenylalanine Degradation Activity In Vivo (PAL)

SYN-PKU517 (comprising 2 chromosomal insertions of PAL (2×fnrS-PAL (malEK, malPT)), and a chromosomal insertion of pheP (fnrS-pheP (lacZ)), thyA auxotrophy (kan/cm)) was compared to SYN-PKU901.

Mice were maintained, fed, and administered phenylalanine as described above. To prepare the bacterial cells for gavage, cells were diluted 1:100 in LB (2 L), grown for 1.5 h aerobically, then shifted to the anaerobe chamber for 4 hours. Prior to administration, cells were concentrated 200× and frozen (15% glycerol, 2 g/L glucose, in PBS). Cells were thawed on ice, and 4e10 cfu/mL was mixed 9:1 in 1M bicarbonate. Each mouse gavaged 800 uL total, or 3.6e10 cfu/mouse.

As described above, blood samples were collected, and the change in phenylalanine concentration as compared to baseline was calculated. The total metabolic activity measured was 39.6 umol/hr. and the total reduction in change in phenylalanine was 17% (P<0.05). These same cells showed an in vitro activity of 1.1 umol/hr./1e9 cells.

Absolute levels of phenylalanine and of PKU and PAL metabolites are consistent with the phenylalanine degradation. For SYN-PKU517, PAL specific metabolites were detected at 4 hours, and moreover, lower levels of PKU specific metabolites were observed as compared to SYN-PKU901, indicating that PAL phenylalanine degradation may cause a shift away from PKU specific metabolites in favor or PAL specific metabolites.

In some embodiments, urine is collected at predetermined time points, and analyzed for phenylalanine levels and levels of PAL and PKU metabolites.

### Example 42. Phenylalanine Degradation Activity in Vivo (PAL)

SYN-PKU705 (comprising 3 chromosomal insertions of PAL (3×fnrS-PAL (malEK, malPT, yicS/nepl)), and 2 chromosomal insertions of pheP (2×fnrS-pheP (lacZ, agaI/rsmI)), and LAAD (driven by the ParaBAD promoter integrated within the endogenous arabinose operon) was compared to SYN-PKU901.

Mice were maintained, fed, and administered phenylalanine as described above. To prepare the bacterial cells for gavage, cells were diluted 1:100 in LB (2 L), grown for 1.5 h aerobically, then shifted to the anaerobe chamber for 4 hours. Prior to administration, cells were concentrated 200× and frozen (15% glycerol, 2 g/L glucose, in PBS). Cells were thawed on ice, and 5e10 cfu/mL was mixed 9:1 in 1M bicarbonate. Each mouse gavaged 800 uL total, or 3.6e10 cfu/mouse. Note: Though this strain contains the LAAD gene, it was not induced in this study

As described above, blood samples were collected, and the change in phenylalanine concentration as compared to baseline was calculated. The total metabolic activity measured was 133.2 umol/hr. and the total reduction in change in phenylalanine was 30% (P<0.05). These same cells showed an in vitro activity of 3.7 umol/hr./1e9 cells.

Absolute levels of phenylalanine and of PKU and PAL metabolites are consistent with the phenylalanine degradation. PAL specific metabolites were detected at 4 hours, and moreover, lower levels of PKU specific metabolites were observed as compared to SYN-PKU901, indicating that PAL phenylalanine degradation may cause a shift away from PKU specific metabolites in favor or PAL specific metabolites, total metabolic activity measured activity was greater than the total metabolic activity measured of the PALS plasmid-based strain SYN-PKU304 and the total reduction in phenylalanine approached that of SYN-PKU304 (30% as compared to 45%).

In some embodiments, urine is collected at predetermined time points, and analyzed for phenylalanine levels and levels of PAL and PKU metabolites.

### Example 43. Phenylalanine Degradation Activity in Vivo (PAL) LAAD

The suitability of P. proteus LAAD for phenylalanine degradation by the genetically engineered bacteria is further assessed in vivo. Bacterial strain SYN-PKU401 (comprising a high copy plasmid comprising LAAD driven by a Tet-inducible promoter is compared to SYN-PKU901.

Mice are maintained, fed, and administered phenylalanine as described above. To prepare the bacterial cells for gavage, cells are diluted 1:100 in LB (2 L), grown for 1.5 h aerobically, then ATC is added and the cells are grown for another 2 hours. Prior to administration, cells are concentrated 200× and frozen for storage. Cells are thawed on ice, and resuspended. Cells are mixed 9:1 in 1M bicarbonate. Each mouse is gavaged four times with 800 uL total volume, or with a total of bacteria ranging from 2×10⁹ to 1×10¹⁰. Blood samples are collected from the mice described in the previous examples and are analyzed for phenylalanine, phenylpyruvate, phenyllactate, trans-cinnamic acid, and hippuric acid levels. Total reduction in phenylalanine and total metabolic activity are calculated.

### Example 44. Generation of DeltaThyA

An auxotrophic mutation causes bacteria to die in the absence of an exogenously added nutrient essential for survival or growth because they lack the gene(s) necessary to produce that essential nutrient. In order to generate genetically engineered bacteria with an auxotrophic modification, the thyA, a gene essential for oligonucleotide synthesis was deleted. Deletion of the thyA gene in E. coli Nissle yields a strain that cannot form a colony on LB plates unless they are supplemented with thymidine.

A thyA::cam PCR fragment was amplified using 3 rounds of PCR as follows. Sequences of the primers used at a 100 um concentration are found in Table 49.

Primer Sequences

| | | | SEQ ID |
|---|---|---|---|
| Name | Sequence | Description | NO |
| SR36 | tagaactgatgcaaaaagtgctcgacgaaggcacacagaTGT | Round 1: binds | SEQ ID |
| | GTAGGCTGGAGCTGCTTC | on pKD3 | NO: 46 |
| SR38 | gtttcgtaattagatagccaccggcgctttaatgcccggaCATA | Round 1: binds | SEQ ID |
| | TGAATATCCTCCTTAG | on pKD3 | NO: 47 |
| SR33 | caacacgtttcctgaggaaccatgaaacagtatttagaactgatgc | Round 2: binds | SEQ ID |
| | aaaaag | to round 1 PCR | NO: 48 |
| | | product | |
| SR34 | cgcacactggcgtcggctctggcaggatgtttcgtaattagatagc | Round 2: binds | SEQ ID |
| | | to round 1 PCR | NO: 49 |
| | | product | |
| SR43 | atatcgtcgcagcccacagcaacacgtttcctgagg | Round 3: binds | SEQ ID |
| | | to round 2 PCR | NO: 50 |
| | | product | |
| SR44 | aagaatttaacggagggcaaaaaaaaccgacgcacactggcgtc | Round 3: binds | SEQ ID |
| | ggc | to round 2 PCR | NO: 51 |
| | | product | |

For the first PCR round, 4×50 ul PCR reactions containing 1 ng pKD3 as template, 25 ul 2×phusion, 0.2 ul primer SR36 and SR38, and either 0, 0.2, 0.4 or 0.6 ul DMSO were brought up to 50 ul volume with nuclease free water and amplified under the following cycle conditions:

step1: 98c for 30 s

step2: 98c for 10 s

step3: 55c for 15 s

step4: 72c for 20 s

repeat step 2-4 for 30 cycles

step5: 72c for 5 min

Subsequently, 5 ul of each PCR reaction was run on an agarose gel to confirm PCR product of the appropriate size. The PCR product was purified from the remaining PCR reaction using a Zymoclean gel DNA recovery kit according to the manufacturer's instructions and eluted in 30 ul nuclease free water.

For the second round of PCR, 1 ul purified PCR product from round 1 was used as template, in 4×50 ul PCR reactions as described above except with 0.2 ul of primers SR33 and SR34. Cycle conditions were the same as noted above for the first PCR reaction. The PCR product run on an agarose gel to verify amplification, purified, and eluted in 30 ul as described above.

For the third round of PCR, 1 ul of purified PCR product from round 2 was used as template in 4×50 ul PCR reactions as described except with primer SR43 and SR44. Cycle conditions were the same as described for rounds 1 and 2. Amplification was verified, the PCR product purified, and eluted as described above. The concentration and purity was measured using a spectrophotometer. The resulting linear DNA fragment, which contains 92 bp homologous to upstream of thyA, the chloramphenicol cassette flanked by frt sites, and 98 bp homologous to downstream of the thyA gene, was transformed into a E. coli Nissle 1917 strain containing pKD46 grown for recombineering. Following electroporation, 1 ml SOC medium containing 3 mM thymidine was added, and cells were allowed to recover at 37 C for 2 h with shaking. Cells were then pelleted at 10,000×g for 1 minute, the supernatant was discarded, and the cell pellet was resuspended in 100 ul LB containing 3 mM thymidine and spread on LB agar plates containing 3 mM thy and 20 ug/ml chloramphenicol. Cells were incubated at 37 C overnight. Colonies that appeared on LB plates were restreaked. +cam 20 ug/ml + or − thy 3 mM. (thyA auxotrophs will only grow in media supplemented with thy 3 mM).

Next, the antibiotic resistance was removed with pCP20 transformation, pCP20 has the yeast Flp recombinase gene, FLP, chloramphenicol and ampicillin resistant genes, and temperature sensitive replication. Bacteria were grown in LB media containing the selecting antibiotic at 37° C. until OD600=0.4-0.6. 1 mL of cells were washed as follows: cells were pelleted at 16,000×g for 1 minute. The supernatant was discarded and the pellet was resuspended in 1 mL ice-cold 10% glycerol. This wash step was repeated 3× times. The final pellet was resuspended in 70 ul ice-cold 10% glycerol. Next, cells were electroporated with 1 ng pCP20 plasmid DNA, and 1 mL SOC supplemented with 3 mM thymidine was immediately added to the cuvette. Cells were resuspended and transferred to a culture tube and grown at 30° C. for 1 hours. Cells were then pelleted at 10,000×g for 1 minute, the supernatant was discarded, and the cell pellet was resuspended in 100 ul LB containing 3 mM thymidine and spread on LB agar plates containing 3 mM thy and 100 ug/ml carbenicillin and grown at 30° C. for 16-24 hours. Next, transformants were colony purified non-selectively (no antibiotics) at 42° C.

To test the colony-purified transformants, a colony was picked from the 42° C. plate with a pipette tip and resuspended in 10 μL LB. 3 μL of the cell suspension was pipetted onto a set of 3 plates: Cam, (37° C.; tests for the presence/absence of CamR gene in the genome of the host strain), Amp, (30° C., tests for the presence/absence of AmpR from the pCP20 plasmid) and LB only (desired cells that have lost the chloramphenicol cassette and the pCP20 plasmid), 37° C. Colonies were considered cured if there is no growth in neither the Cam or Amp plate, picked, and re-streaked on an LB plate to get single colonies, and grown overnight at 37° C.

### Example 45. Phenylalanine Quantification (Dansyl-Chloride Derivatization)

For in vitro and in vivo assays described herein, which assess the ability of the genetically engineered bacteria to degrade phenylalanine and which require quantification of phenylalanine levels in the sample, a dansyl-chloride derivatization protocol was employed as follows.

### Sample Preparation

Phenylalanine standards (1000, 500, 250, 100, 20, 4 and 0.8 μg/mL in water) were prepared. On ice, 10 μL of sample was pipetted into a V-bottom polypropylene 96-well plate, and 190 μL of 60% acetonitrile with 1 ug/mL of L-Phenyl-d₅-alanine internal standard was added. The plate was heat sealed, mixed well, and centrifuged at 4000 rpm for 5 min. Next, 5 μL of diluted samples were added to 95 μL of derivatization mix (85 μL 10 mM NaHCO₃ pH 9.7 and 10 μL 10 mg/mL dansyl-chloride (diluted in acetonitrile)) in a V-bottom 96-well polypropylene plate, and the plate was heat-sealed and mixed well. The samples were incubated at 60° C. for 45 min for derivatization and then centrifuged at 4000 rpm for 5 minutes. Next, 20 μL of the derivatized samples were added to 180 μL of water with 0.1% formic acid in a round-bottom 96-well plate, plates were heat-sealed and mixed well.

LC-MS/MS Method

Phenylalanine was measured by liquid chromatography coupled to tandem mass spectrometry (LC-MS/MS) using a Thermo TSQ Quantum Max triple quadrupole mass spectrometer. HPLC Method details are described in Tables below. Tandem Mass Spectrometry details are described below.

HPLC Method Details

| | |
|---|---|
| Column | Luna C18(2) column, 5 μm (50 × 2.1 mm) |
| Mobile Phase A | 100% H2O, 0.1% Formic Acid |
| Mobile Phase B | 100% ACN, 0.1% Formic Acid |
| Injection volume | 10 uL |

HPLC Method Details

| Total Time | Flow Rate | | |
|---|---|---|---|
| (min) | (μL/min) | A % | B % |
| 0 | 400 | 90 | 10 |
| 0.5 | 400 | 90 | 10 |
| 0.6 | 400 | 10 | 90 |
| 2 | 400 | 10 | 90 |
| 2.01 | 400 | 90 | 10 |
| 3 | 400 | 90 | 10 |

Tandem Mass Spectrometry Details

| | |
|---|---|
| Ion Source | HESI-II |
| Polarity | Positive |

SRM transitions

| | |
|---|---|
| L-Phenylalanine | 399.1/170.1 |
| L-Phenyl-d5-alanine | 404.1/170.1 |

### Example 46. Trans-Cinnamic Acid Quantification (Trifluoroethylamine Derivatization)

For in vitro and in vivo assays described herein, which assess the ability of the genetically engineered bacteria to degrade phenylalanine and which require quantification of Trans-cinnamic acid levels in the sample, a trifluoroethylamine derivatization protocol was employed as follows.

b. Sample Preparation

Trans-cinnamic acid standard (500, 250, 100, 20, 4 and 0.8 μg/mL in water) were prepared. On ice, 10 μL of sample was pipetted into a V-bottom polypropylene 96-well plate. Next, 30 μL of 80% acetonitrile with 2 ug/mL of trans-cinnamic acid-d7 internal standard was added, and the plate was heat sealed, mixed well, and centrifuged at 4000 rpm for 5 minutes. Next, 204, of diluted samples were added to 180 μL of 10 mM MES pH4, 20 mM N-(3-Dimethylaminopropyl)-N′-ethylcarbodiimide (EDC), 20 mM trifluoroethylamine in a round-bottom 96-well polypropylene plate. The plate was heat-sealed, mixed well, and samples were incubated at room temperature for 1 hour.

c. LC-MS/MS Method

Trans-cinnamic acid was measured by liquid chromatography coupled to tandem mass spectrometry (LC-MS/MS) using a Thermo TSQ Quantum Max triple quadrupole mass spectrometer. HPLC Method details are described in Tables below. Tandem Mass Spectrometry details are described.

HPLC Method Details

| | |
|---|---|
| Column | Thermo Aquasil C18 column, 5 μm (50 × 2.1 mm) |
| Mobile Phase A | 100% H2O, 0.1% Formic Acid |
| Mobile Phase B | 100% ACN, 0.1% Formic Acid |
| Injection volume | 10 uL |

HPLC Method Details

| Total Time | | | |
|---|---|---|---|
| (min) | Flow Rate (μL/min) | A % | B % |
| 0 | 500 | 100 | 0 |
| 1 | 500 | 100 | 0 |
| 2 | 500 | 10 | 90 |
| 4 | 500 | 10 | 90 |
| 4.01 | 500 | 100 | 0 |
| 5 | 500 | 100 | 0 |

Tandem Mass Spectrometry Details

| | |
|---|---|
| Ion Source: | HESI-II |
| Polarity | Positive |

SRM transitions

| | |
|---|---|
| Trans-cinnamic acid: | 230.1/131.1 |
| Trans-cinnamic acid-d7 | 237.1/137.2 |

### Example 47. Phenylalanine, Trans-Cinnamic Acid, Phenylacetic Acid, Phenylpyruvic Acid, Phenyllactic Acid, Hippuric Acid and Benzoic Acid Quantification (2-Hydrazinoquinoline Derivatization)

For in vitro and in vivo assays described herein, which assess the ability of the genetically engineered bacteria to degrade phenylalanine and which require quantification of phenylalanine, trans-cinnamic acid, phenylacetic acid, phenylpyruvic acid, phenyllactic acid, hippuric acid, and benzoic acid levels in the sample, a 2-Hydrazinoquinoline derivatization protocol was employed as follows

d. Sample Preparation

Standard solutions containing 250, 100, 20, 4, 0.8, 0.16 and 0.032 μg/mL of each standard in water were prepared. On ice, 10 μL of sample was pipetted into a V-bottom polypropylene 96-well plate, and 90 μL of the derivatizing solution containing 50 mM of 2-Hydrazinoquinoline (2-HQ), dipyridyl disulfide, and triphenylphospine in acetonitrile with 1 ug/mL of L-Phenyl-d₅-alanine, 1 ug/mL of hippuric acid-d5 and 0.25 ug/mL trans-cinnamic acid-d7 internal standards was added. The plate was heat-sealed, mixed well, and samples were incubated at 60° C. for 1 hour for derivatization, and then centrifuged at 4000 rpm for 5 min. In a round-bottom 96-well plate, 20 μL of the derivatized samples were added to 180 μL of water with 0.1% formic acid. Plates were heat-sealed and mixed well.

e. LC-MS/MS Method

Metabolites derivatized by 2-HQ were measured by liquid chromatography coupled to tandem mass spectrometry (LC-MS/MS) using a Thermo TSQ Quantum Max triple quadrupole mass spectrometer. HPLC details are described in Tables below. Tandem Mass Spectrometry details are described.

HPLC Method Details

| | |
|---|---|
| Column | Luna C18(2) column, 3 μm (150 × 2.1 mm) |
| Mobile Phase A | 100% H2O, 0.1% Formic Acid |
| Mobile Phase B | 100% ACN, 0.1% Formic Acid |
| Injection volume | 10 uL |

HPLC Method Details

| Total Time | Flow Rate | | |
|---|---|---|---|
| (min) | (μL/min) | A % | B % |
| 0 | 500 | 90 | 10 |
| 0.5 | 500 | 90 | 10 |
| 2 | 500 | 10 | 90 |
| 4 | 500 | 10 | 90 |
| 4.01 | 500 | 90 | 10 |
| 4.25 | 500 | 90 | 10 |

Tandem Mass Spectrometry Details

| | |
|---|---|
| Ion Source | HESI-II |
| Polarity | Positive |

SRM transitions

| | |
|---|---|
| L-Phenylalanine: | 307.1/186.1 |
| L-Phenyld5-alanine | 312.1/186 |
| Trans-cinnamic acid | 290.05/131.1 |
| Trans-cinnamic acid-d7 | 297.05/138.1 |
| Hippuric acid | 321.1/160.1 |
| Hippuric acid-d5 | 326/160 |
| Phenylacetic acid | 278.05/160.1 |
| Phenyllactic acid | 308.05/144.1 |
| Benzoic acid | 264.05/105.1 |
| Phenylpyruvate | 306.05/260.1 |

### Example 48. Effect of PAL Copy Number, pheP, and LAAD on Strain Activity

To illustrate the effect of pheP, various copy numbers of PAL, and the further addition of LAAD on the rate of phenylalanine degradation in vitro, strains containing different copy numbers of PAL, either in the presence or absence of pheP and LAAD were compared sided by side in an in vitro phenylalanine consumption assay.

The genetically engineered bacteria were grown overnight, diluted and allowed to grow for another 2.5 hours in the absence or presence of 0.1% arabinose (if the construct comprises LAAD). Cultures were then placed in Coy anaerobic chamber supplying 90% N2, 5% CO2, and 5% H2 for 4 hours in phenylalanine containing medium (4 mM phenylalanine). Whole cell extracts were prepared and phenylalanine was quantified by mass spectrometry and rates were calculated.

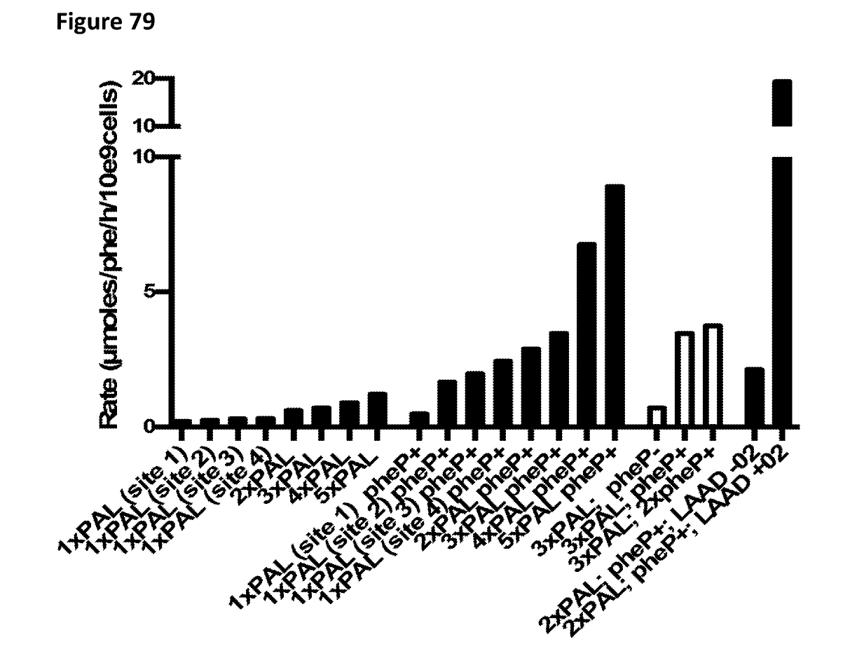

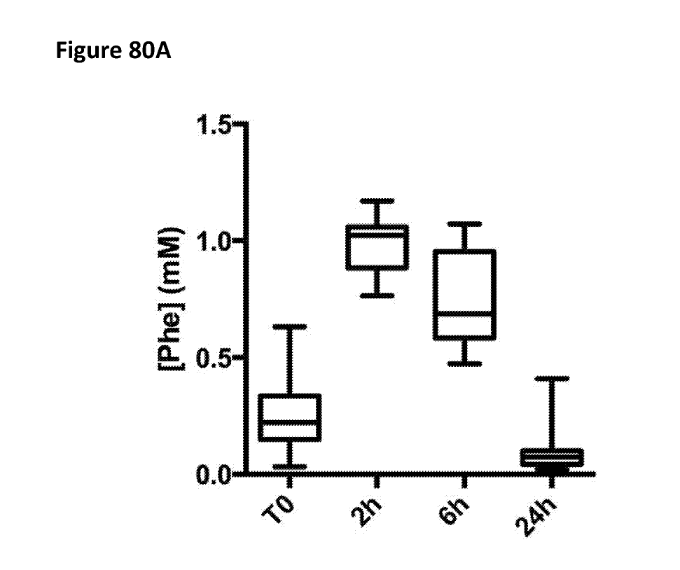

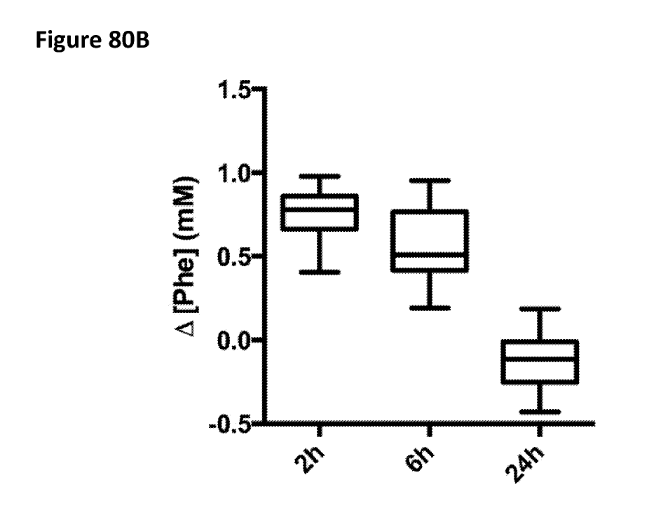

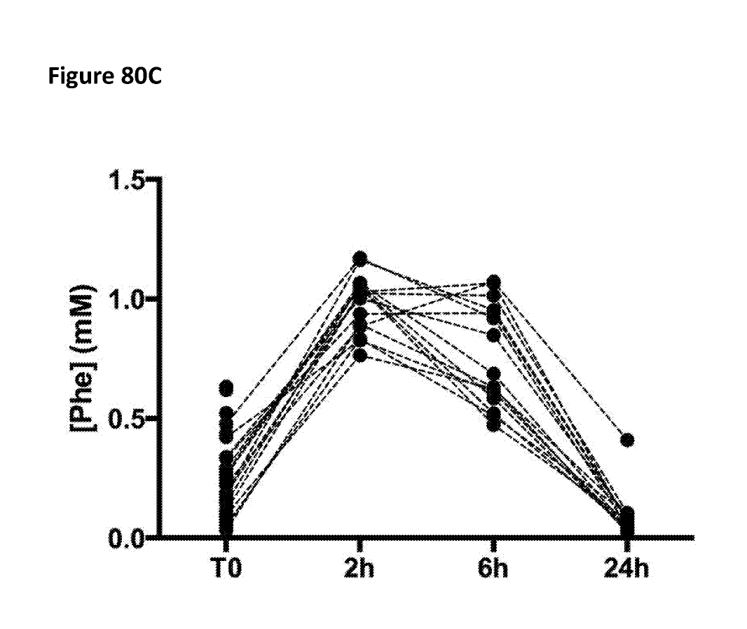

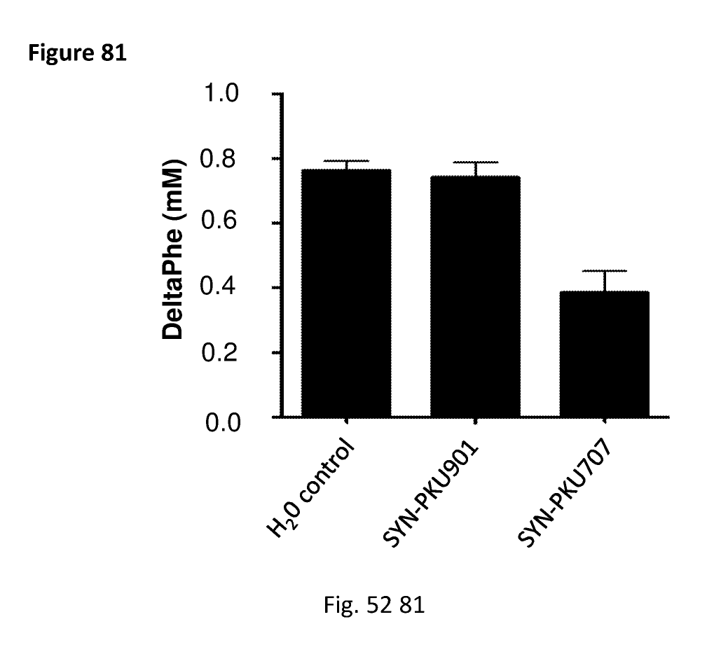

Results shown in FIG. 79 demonstrate that increasing the copy number of PAL increases the rate of phenylalanine degradation. Addition of the high affinity transporter pheP abrogates the transport limitation, allowing greater PAL activity. The transporter copy number does not increase rate (PAL, and not transport (pheP), is limiting). In the presence of oxygen, LAAD can degrade Phe at an extremely high rate.

### Example 49. PAL-specific Metabolite Detection in SYN-PKU706 in Blood and Urine

To evaluate levels of TCA and hippuric acid in the urine, and to assess the utility of TCA and hippuric acid measurements as an indicator of strain activity, levels of TCA and hippuric acid were measured in serum and urine in an in vivo mouse model (BTBR-Pahenu2 mice) following subcutaneous phenylalanine challenge. SYN-PKU706 (comprising three copies of fnrS-PAL (integrated at MalP/T, HA3/4, and MalE/K), 2 copies of fnr-PheP (integrated at HA1/2 and LacZ), and one copy of Para-LAAD (LAAD knocked into the arabinose operon (Para::LAAD)), was compared to wild type Nissle with a streptomycin resistance (SYN-PKU901) in this study.

To prepare the cells, cells were diluted 1:100 in LB (2 L), grown for 1.5 h aerobically in the presence of 0.1% arabinose, then shifted to the anaerobe chamber for 4 hours. Prior to administration, cells were concentrated and frozen (15% glycerol, 2 g/L glucose, in PBS). Cells were thawed on ice, mixed 9:1 in 1M bicarbonate. Each mouse gavaged 750 uL total, or 1×10e11 cfu/mouse total over 3 gavages.

Beginning 4 days prior to the study (i.e., Days −4-1), Pah ENU2/2 mice (˜11-15 weeks of age) were maintained on phenylalanine-free chow and water that was supplemented with 0.5 grams/L phenylalanine. On Day 1, mice were randomized into treatment groups and blood samples were collected by sub-mandibular skin puncture to determine baseline phenylalanine levels. Mice were also weighed to determine the average weight for each group. Mice were then administered single dose of phenylalanine by subcutaneous injection at 0.1 mg per gram body weight, according to the average group weight. At 1, 2 and 3 h post Phe challenge, the bacteria were administered to mice by oral gavage (3×250 ul; 1×10e11 cfu/mouse total over 3 gavages) as follows: SYN-PKU901 (streptomycin resistant Nissle n=12), SYN-PKU-706 (n=12). Sodium bicarbonate was added to final concentration of 100 mM for both strains. H₂O only was administered as control (n=12). Animals were bled and urine was collected from all animals at 4 h post Phe challenge. Blood was stored on ice for LC/MS analysis.

TCA and Hippuric acid were measured. Both serum and urine levels of TCA and Hippuric acid were increased in SYN-PKU-706 over SYN-PKU-901 as compared to the H2O controls. Similar levels of metabolites were measured in urine when administering other efficacious PKU strains. Low levels of TCA were present in both urine and serum. Lower levels of hippuric acid were detected in serum. Highest levels were detected for hippuric acid in urine, indicating that the majority of TCA generated by the bacteria is converted to hippuric acid in the liver and is excreted in the urine. These and other results described herein indicate that levels of hippuric acid in the urine can be used as an indicator or biomarker of PAL activity.

### Example 50. Hippuric Acid Measurement as a Method to Measure In Vivo Cell Activity

To determine whether hippuric acid detection in the urine is suitable as a measure for in vivo cell activity and a potential biomarker, the extent of turnover of TCA into hippuric acid was assessed by oral gavage of various concentrations of TCA in PKU mice and subsequent measurement of TCA and hippuric acid by LC/MS.

On day 1 of the study, Pah ENU2/2 mice (˜8-10 weeks) were randomized into TCA challenge treatment groups as follows: Group 1: 0.1 mg/g TCA (n=6); Group 2: 0.05 mg/g TCA (n=6); Group 3: 0.025 mg/g TCA (n=6); Group 4: 0.0125 mg/g TCA (n=6); Group 5: H₂O Control (n=6). Various TCA concentrations were administered by oral gavage.

Animals were transferred to metabolic cages (3 mice per cage, 2 cages per group) and urine and feces were collected at for 4 h post TCA dose. Urine and feces were transferred to appropriate tubes and store samples on ice until processed for MS analysis. The amount of TCA and hippuric acid recovered upon oral gavage of 0.0125, 0.025, 0.05, or 0.1 mg/g TCA at 4 hours after gavage. Insignificant amounts of TCA and hippurate were detected in blood and feces (data not shown). A nearly full recovery of TCA in the form of hippurate was observed in the urine. As a result, 1 mol of hippurate found in the urine would equal 1 mol of Phe converted to TCA in the small intestine in a PKU mouse upon administration of a PKU strain.

Next, the kinetics of conversion of TCA to hippuric acid was assessed in a time course post pure TCA oral gavage. On day 1 of the study, Pah ENU2/2 mice (˜8-10 weeks) were randomized into TCA challenge treatment groups as follows: Group 1: 0.033 mg/g TCA (n=6); Group 2: 0.1 mg/g TCA (n=6); Group 3: H₂O Control (n=6). TCA concentrations were administered by oral gavage. Animals were transferred to metabolic cages (3 mice per cage, 2 cages per group) and urine samples were collected at 1, 2, 3, 4, 5, 6 hours post TCA dose. Urine was transferred to appropriate tubes and store samples on ice until processed for MS analysis. As seen in FIG. 46, at the high dose TCA gavage, TCA is converted to hippurate and excreted in the urine by 4 hours.

### Example 51. Generation of Additional PKU Strains

The following PKU strains were generated for use in subsequent examples.

SYN-PKU707 comprises three chromosomal insertions of PAL3 (3×fnrS-PAL (malP/T, yicS/nepl, malE/K)) and two copies of pheP (2×fnrS-pheP (lacZ, agaI/rsmI)). SYN-PKU707 further comprises one copy of the mutated FNR transcription factor FNRS24Y (Para::FNRS24Y). SYN-PKU712 essentially corresponds to SYN-PKU707 with a dapA auxotrophy.

SYN-PKU708 comprises a bacterial chromosome with three chromosomal insertions of PAL3 (3×fnrS-PAL (malP/T, yicS/nepl, malE/K)) and two copies of pheP (2×fnrS-pheP (lacZ, agaI/rsmI)). SYN-PKU708 further comprises one copy of the mutated FNR transcription factor FNRS24Y (Para::FNRS24Y) and one copy of LAAD inserted at the same insertion site (the arabinose operon), which is transcribed as a bicistronic message from the endogenous arabinose promoter. The genome is further engineered to include a dapA auxotrophy, in which the dapA gene is deleted. SYN-PKU711 essentially corresponds to SYN-PKU708 without a dapA auxotrophy.

SYN-PKU709 comprises a bacterial chromosome comprising three chromosomal insertions of PAL3 (3×fnrS-PAL (malP/T, yicS/nepl, malE/K)) and two copies of pheP (2×fnrS-pheP (lacZ, agaI/rsmI)). SYN-PKU709 further comprises one copy of the LAAD inserted into the arabinose operon with expression driven by the native Para promoter (Para::LAAD). The genome is further engineered to include a dapA auxotrophy, in which the dapA gene is deleted.

SYN-PKU710 comprises a bacterial chromosome comprising three chromosomal insertions of PAL3 (3×fnrS-PAL (malP/T, yicS/nepl, malE/K)) and two copies of pheP (2×fnrS-pheP (lacZ, agaI/rsmI)). SYN-PKU710 further comprises one copy of the LAAD inserted into the arabinose operon with expression driven by the native Para promoter (Para::LAAD). SYN-PKU710 further comprises two copies of IPTG inducible PAL3 (2×LacIPAL, exo/cea and rhtC/rhtB), a dapA auxotrophy and is cured of all antibiotic resistances. Constructs and methods for the generation of these strains are described herein. Additional constructs needed for strain construction are generated according to methods described herein, e.g., Examples 1, 2, 22, and 23 and are shown in Table 70, Table 71, and Table 72.

### Example 52. Analysis of Blood Phe Levels and Metabolic Conversion to Hippurate Following Administration of PKU Strain SYN-PKU-707

The ability of engineered probiotic strain SYN-PKU-707 to convert phenylalanine to hippurate was assessed. Strain SYN-PKU-707 comprises three copies of PAL driven by the FNR promoter (inserted into the chromosome at the malE/K, yicS/nepl, an dmalP/T loci), and two copies of pheP driven by the FNR promoter (inserted into the chromosome at the LacZ and agaI/rsmI loci), and the mutant FNRS24Y.

Cultures (1:100 back-dilutions from overnight cultures) were grown to early log phase for 1.5 h before the addition of L-arabinose at 0.15% final concentration for induction. Cultures were induced for 4 hours (aerobically). Prior to administration, cells were concentrated 200× and frozen (10% glycerol, 2 g/L glucose, in PBS). Cells were thawed on ice, and mixed 9:1 in 1M bicarbonate. Each mouse was gavaged 750 uL total, or 1×10e11 cfu/mouse.

Beginning 4 days prior to the study (i.e., Days −4-1), Pah ENU2/2 mice (˜11-15 weeks of age) were maintained on phenylalanine-free chow and water that was supplemented with 0.5 grams/L phenylalanine. On Day 1, mice were randomized into treatment groups according to weight as follows: Group 1: SYN-PKU901 (n=9); Group 2: SYN-PKU-707 (n=9); Group 3: H₂O Control (n=9). Blood samples were collected by sub-mandibular skin puncture to determine baseline phenylalanine levels. Mice were then administered single dose of phenylalanine by subcutaneous injection at 0.1 mg per gram body weight, according to the average group weight. At 1, 2 and 3 h post Phe challenge, the bacteria (or water) were administered to mice by oral gavage (3×250 ul; 1×10e11 total bacteria). Sodium bicarbonate was added to final concentration of 100 mM for both strains. Animals were bled and urine was collected from all animals up to 4 h post Phe challenge. All treatment groups were bled at 4 h post phenylalanine challenge. Blood was stored on ice for LC/MS analysis. Results in FIG. 81 show that SYN-PKU707 was efficacious in reducing blood phenylalanine and that hippurate can be found specifically excreted in the cages of mice treated with SYN-PKU707, indicating that the cells are active in vivo.

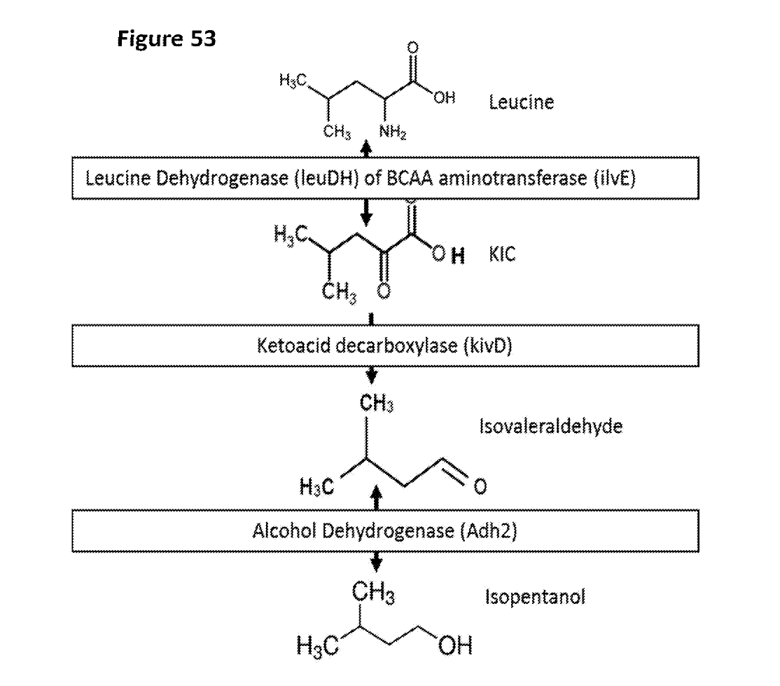

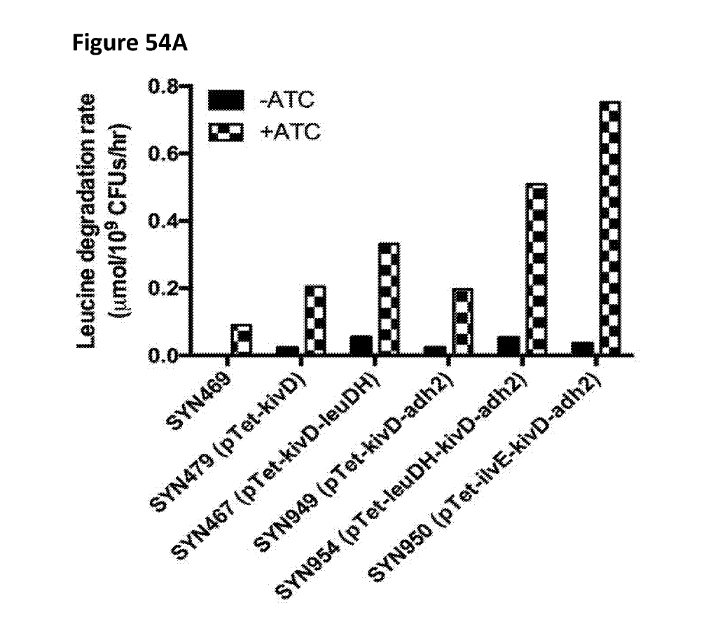

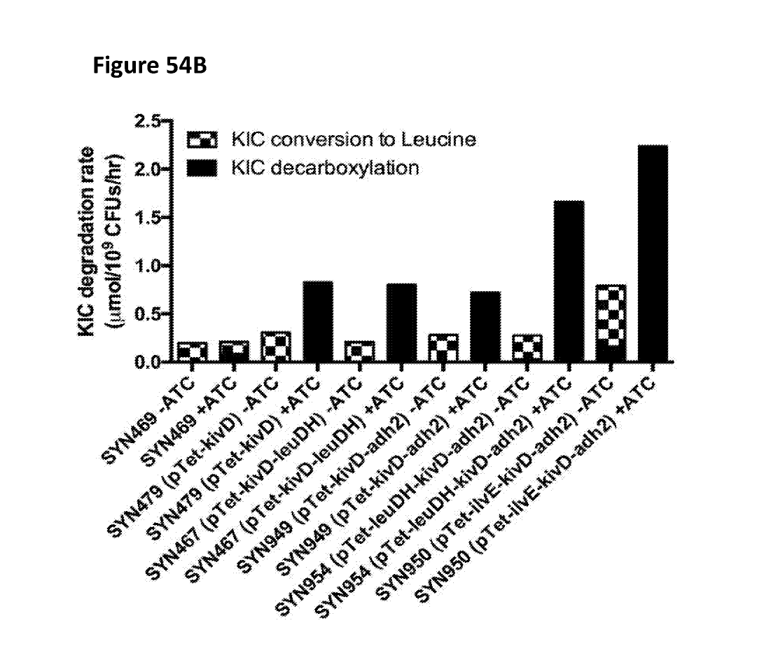

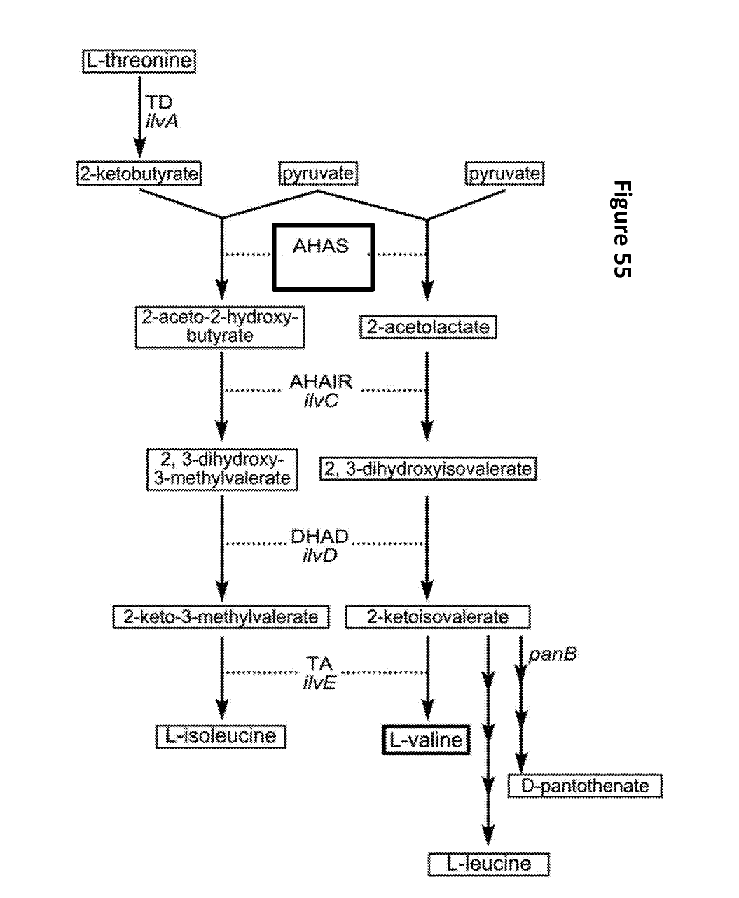

FIG. 81 depicts blood phenylalanine concentrations relative to baseline; total metabolic activity for SYN-PKU707 was calculated as 269 umol/hr and the total reduction in Δphe was =49% (P<0.05) relative to SYN-PKU901 (P<0.05). FIG. 53 depicts the urine hippurate concentration at 4 hours post phenylalanine injection. In FIG. 53, levels for each cage (three mice per cage) are shown separately and numbered 1-3 for each strain. These results indicate that approximately 15-20% of injected phenylalanine is converted to hippurate. Phenylalanine is converted to TCA in the small intestine, which is then converted into hippurate in the liver.

### Example 53. Hippuric Acid Recovered in Urine Following a Single Dose PKU Strain SYN-PKU707

To determine how cell numbers gavaged in a single gavage affect recovery of hippuric acid in the urine, PKU mice were gavaged with a single dose of SYN-PKU707 at various doses and hippuric acid levels were monitored over a period of 6 hours post-gavage. Strain SYN-PKU-707 comprises three copies of PAL driven by the FNR promoter (inserted into the chromosome at the malE/K, yicS/nepl, an dmalP/T loci), and two copies of pheP driven by the FNR promoter (inserted into the chromosome at the LacZ and agaI/rsml loci), and the mutant FNRS24Y.

To prepare the cells, cells were diluted 1:100 in LB (2 L), grown for 1.5 h aerobically, then induced aerobically in the presence of 0.15% arabinose for 4 hours. Prior to administration, cells were concentrated 200× and frozen (10% glycerol, 2 g/L glucose, in PBS). Cells were thawed on ice. Each mouse was gavaged 750 uL total, at 3e10, 6e6, 1.2e9, and 2.4e8 cfu/mouse.

Briefly, beginning 4 days prior to the study, Pah ENU2/2 mice (˜11-15 weeks of age) were maintained on phenylalanine-free chow and water that was supplemented with 0.5 grams/L phenylalanine. On Day 1, mice were randomized into treatment groups according to weight into groups as follows: Group 1: SYN-PKU-707 (n=6); Group 2: H2O Control (n=6). Blood samples were collected by sub-mandibular skin puncture to determine baseline phenylalanine levels. Mice were then administered single dose of phenylalanine by subcutaneous injection at 0.1 mg per gram body weight, according to the average group weight. At 1, 2 and 3 h post Phe challenge, the bacteria were administered to mice by oral gavage (3×250 ul) at various doses (3e10, 6e9, 1.2e9, and 2.4e8). Sodium bicarbonate was added to final concentration of 100 mM for both strains. Urine was collected from all animals up to at 1, 2, 3, 4, 5, and 6 hours post phenylalanine challenge, and total amounts of hippuric acid recovered at each time point was determined by LC/MS, the absolute amount of hippuric acid recovered in urine over the 6 hour time frame, as determined by mass spectroscopy. A dose-dependent increase in hippurate recovered in the urine of mice was observed.

### Example 54. In Vitro Activity of SYN-PKU709, SYN-PKU707, and SYN-PKU708

A 1:100 back-dilution from overnight culture of SYN-PKU709—was grown to early log phase for 1.5 h before moving to the anaerobic chamber for 4 hours for induction as described herein. To perform activity assay, 1e8 cells were resuspended and incubated in assay buffer (M9 media with 0.5% glucose, 50 mM Phe, and 50 mM MOPS). Supernatant samples were taken over time and TCA (the product of PAL) was measured by absorbance at 290 nm to determine the rate of TCA production/PAL activity.

For strains possessing FNRS24Y (SYN-PKU707 and SYN-PKU708): Cultures (1:100 back-dilutions from overnight cultures) were grown to early log phase for 1.5 h before the addition of L-arabinose at 0.15% final concentration for induction. Cultures were induced for 4 hours (aerobically). To perform activity assay, 1e8 cells were resuspended and incubated in assay buffer (M9 media with 0.5% glucose, 50 mM Phe, and 50 mM MOPS). Supernatant samples were taken over time and TCA (the product of PAL) was measured by absorbance at 290 nm to determine the rate of TCA production/PAL activity.

All cultures shared the same level of PAL activity, 4 umol TCA produced/hr/1e9 cells.

### Example 55. Determination of the Kinetics of Phenylalanine in the Enterocirculation Model

To characterize the phenylalanine enterocirculation model, first the kinetics of serum levels of phenylalanine post phenylalanine challenger were assessed.

On day −6, PKU (enu2) mice were placed on phenylalanine-free chow and water (+)Phe (0.5 g/L)

On day 1, animals were bled to get T=0 (pre Phe challenge), and animals were randomized into two groups based on phenylalanine levels measured. The first treatment group (n=15) included mice for use for the 2, 6 and 24 hour post phenylalanine challenge time points; Group 2 (n=15) included mice for the 4 and 8 hour post phenylalanine challenge time points.

Mice were pre-weighed to obtain the average weight for each group. Phenylalanine was dosed at a concentration equal to average group weight. Animals were dosed subcutaneously with 0.1 mg/g phenylalanine. In Group 1, animals were bled at 2, 6 and 24 h post Phe challenge. In Group 2, animals were bled at 4 and 8 h post Phe challenge. Whisker plots in distribution of mouse blood phenylalanine levels (both overall phenylalanine levels and change in phenylalanine levels from T0. Phenylalanine levels were stably elevated over at least a 6 hour period.

Next, subcutaneous 13C-Phe challenge was performed to determine extent of recirculation in our PKU (enu2) mice.

On day −6, PKU mice were placed on phenylalanine-free chow and water (+)Phe (0.5 g/L). Animals were pre-weighed to obtain average weight for each group. On day 1, animals were bled to obtain T=0 (pre Phe challenge). Animals were randomized into three treatment groups based on phenylalanine levels measured. Groups (n=2 per group) were as follows: Group 1=0 min (no Phe challenge); Group 2=20 min post Phe challenge time point; Group 3=2 h post Phe challenge time point Animals were dosed phenylalanine at a concentration equal to average group weight at a subcutaneous dose of 0.1 mg/g Phe. Group 1 (0 min group) did not receive a phenylalanine dose. At each time point post Phe Challenge (20 min and 2 h), animals were bled and euthanized, the GI tract was removed and sectioned into small and large intestines.

For the T=0 min group, organ harvest is in absence of any Phe challenge. Sections were flushed sections with ˜1 ml cold PBS and effluent was collected in 1 ml microfuge tubes, and samples were stored on ice. Consequently, all intestinal effluents approximately 2.5× diluted in the measurements due to the intestinal PBS flush, indicating absolute levels in vivo are likely be higher than shown).

isotopic Phe injected subcutaneously (SC) is seen in the small intestine within 20 min, and enterorecirculation of labeled 13C-Phe was confirmed to occur.

Next the overall amino acid levels were measured in blood, small and large intestine in wild type and PKU mice.

On day 1, animals were fasted for 1 h, and bled to obtain T=0. Animals were euthanized and organs were harvested for each animal. The GI tract was removed and sectioned into small and large intestines. Sections were flushed with ˜1 ml cold PBS and effluent was collected in 1 ml microfuge tubes. Samples were stored on ice (blood and intestinal effluents) until LC-MS analysis, phenylalanine levels were high in enu2 blood, but no other major differences between WT and enu2 mice were observed. Example 48. In vivo Administration and Efficacy of SYN-PKU708 at Various Doses

The ability of engineered probiotic strain SYN-PKU-708 change levels of phenylalanine post SC injection and to convert phenylalanine to hippurate was assessed at various doses. Strain SYN-PKU-707 comprises three copies of PAL driven by the FNR promoter (inserted into the chromosome at the malE/K, yicS/nepl, an dmalP/T loci), and two copies of pheP driven by the FNR promoter (inserted into the chromosome at the LacZ and agaI/rsmI loci), and the mutant FNRS24Y-LAAD knocked into the arabinose operon, which is transcribed as a bicistronic message.

Cultures (1:100 back-dilutions from overnight cultures) were grown to early log phase for 1.5 h before the addition of L-arabinose at 0.15% final concentration for induction. Cultures were induced for 4 hours (aerobically). Prior to administration, cells were concentrated 200× and frozen (10% glycerol, 2 g/L glucose, in PBS). Cells were thawed on ice, and mixed 9:1 in 1M bicarbonate. Each mouse was gavaged 750 uL total, or 5.3×10e11, 1.8×10e11, 6×10e11, 2×10e9 cfu/mouse.

Beginning 4 days prior to the study (i.e., Days ˜4-1), Pah ENU2/2 mice (˜11-15 weeks of age) were maintained on phenylalanine-free chow and water that was supplemented with 0.5 grams/L phenylalanine. On Day 1, mice were randomized into treatment groups according to weight as follows: Group 1: SYN-PKU-708 (n=9; dosing with 5.3×10e11 CFU); Group 2: SYN-PKU-708 (n=6; dosing with 1.8×10e11 CFU); Group3: SYN-PKU-708 (n=6; dosing with 6×10e11 CFU); Group 4: SYN-PKU-708 (n=6; dosing with 2×10e9 CFU); Group 5: H₂O Control (n=6).

Animals were transferred to metabolic cages (3 mice per cage, 3 cages per group) and blood samples were collected by sub-mandibular skin puncture to determine baseline phenylalanine levels. Mice were then administered single dose of phenylalanine by subcutaneous injection at 0.1 mg per gram body weight, according to the average group weight. At 1, 2 and 3 h post Phe challenge, the bacteria (or water) were administered to mice by oral gavage (3×250 ul; 5.3×10e11, 1.8×10e11, 6×10e11, 2×10e9 cfu/mouse). Sodium bicarbonate was added to final concentration of 100 mM for both strains Animals were bled and urine was collected from all animals up to 4 h post Phe challenge. All treatment groups were bled at 4 h post phenylalanine challenge. Blood and urine was stored on ice for LC/MS analysis.

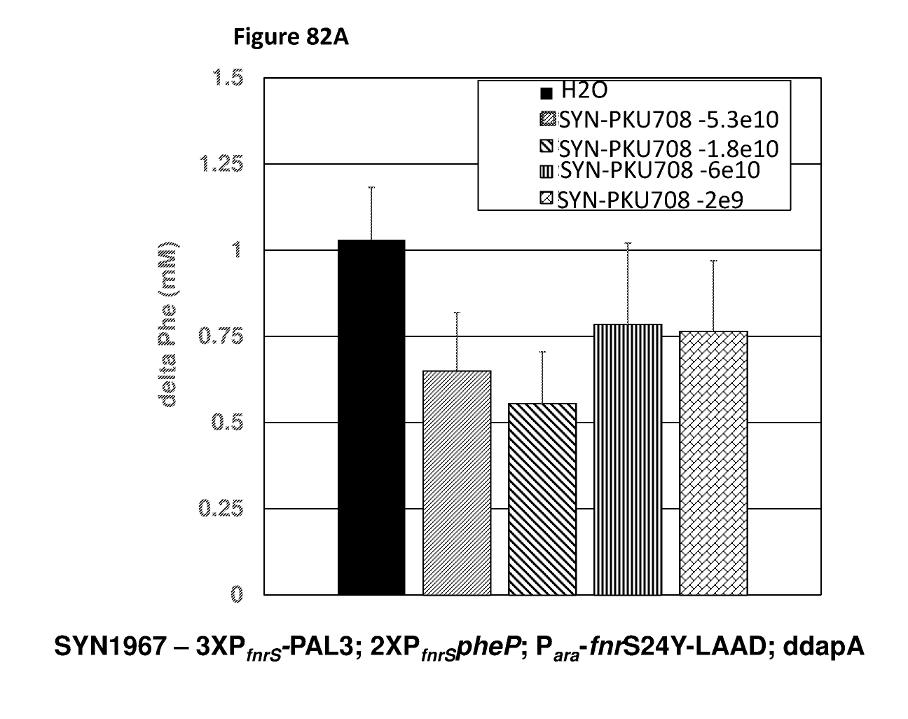

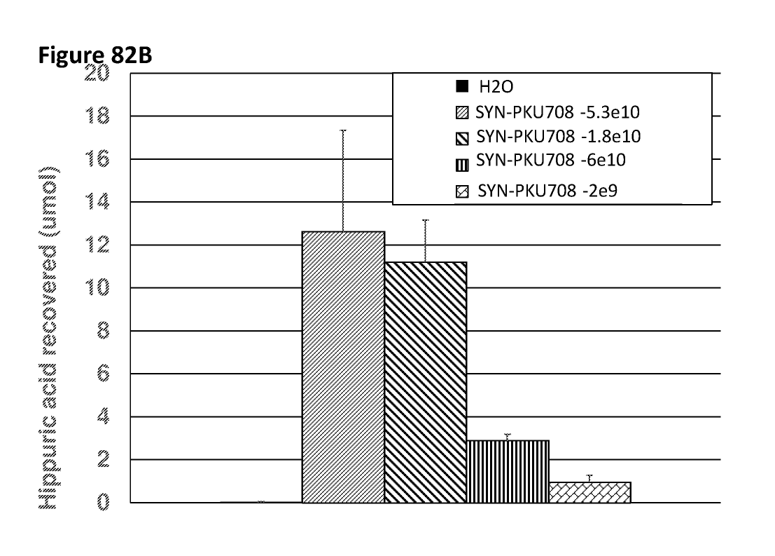

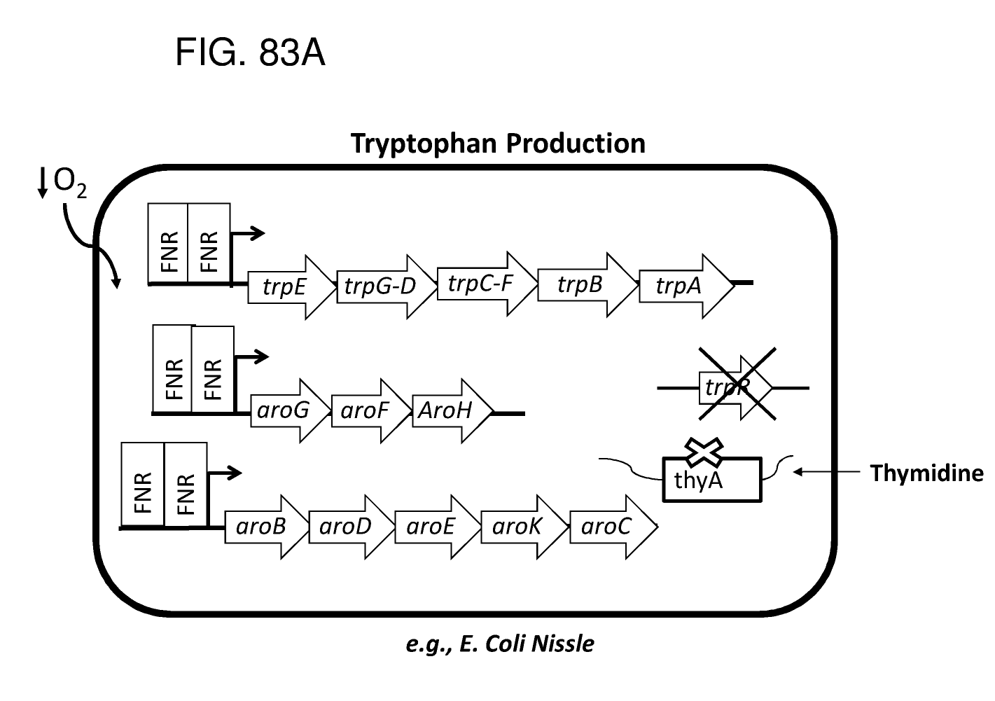

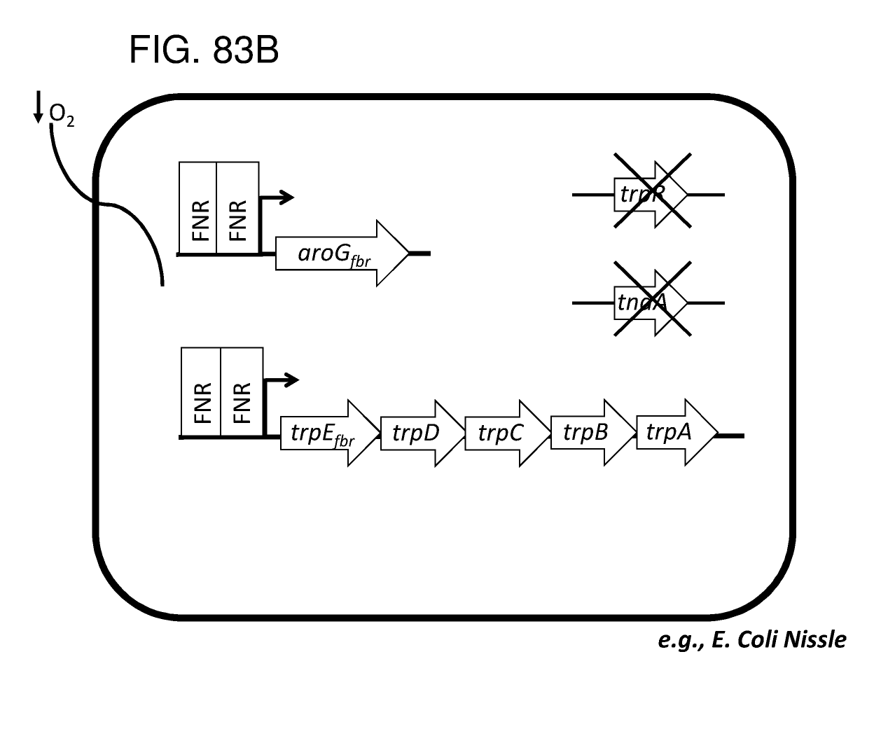

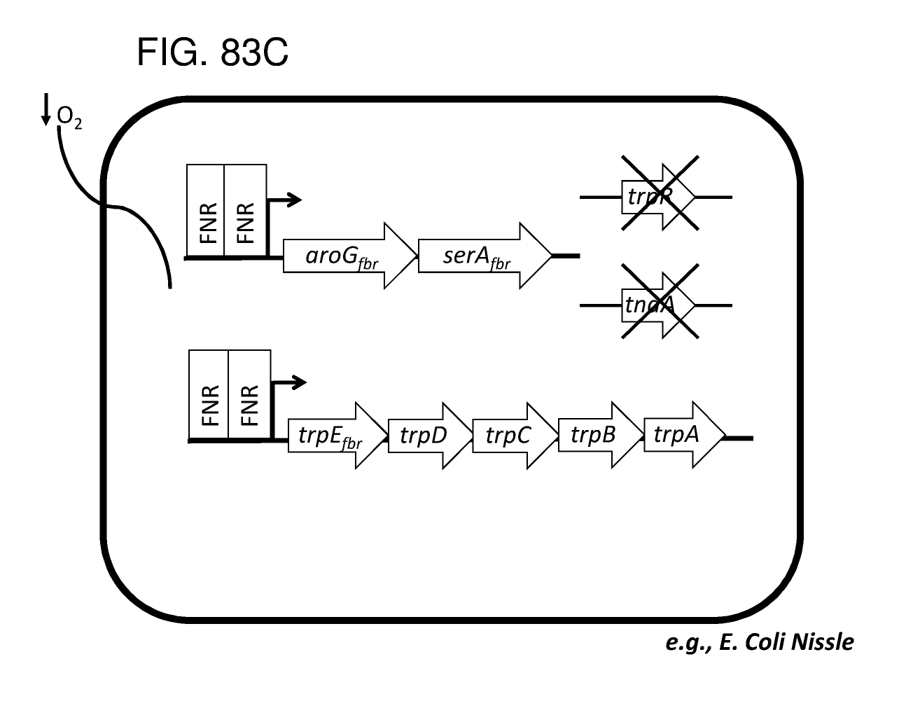

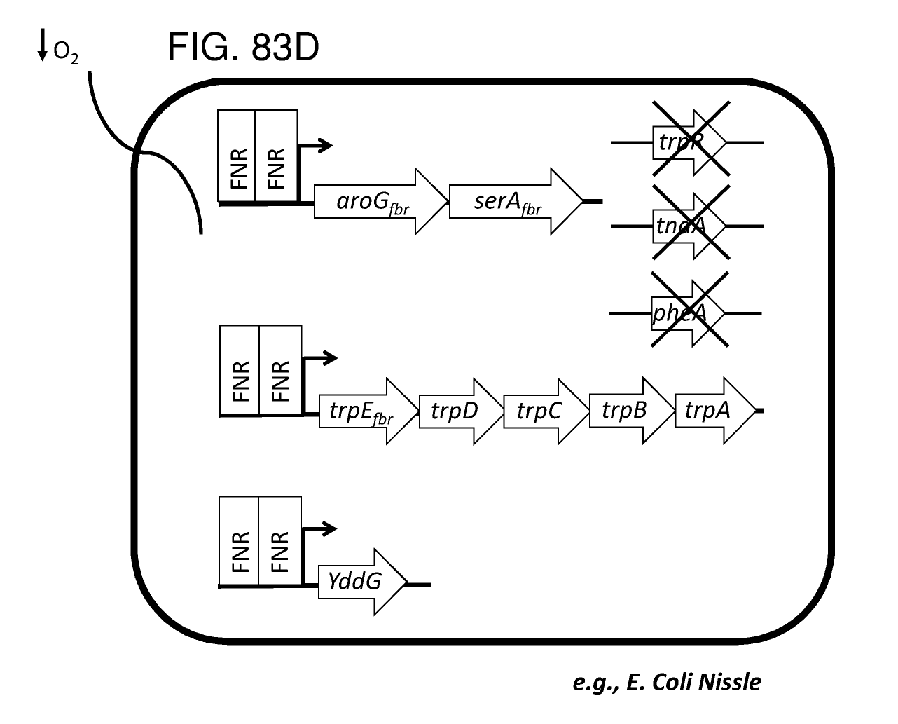

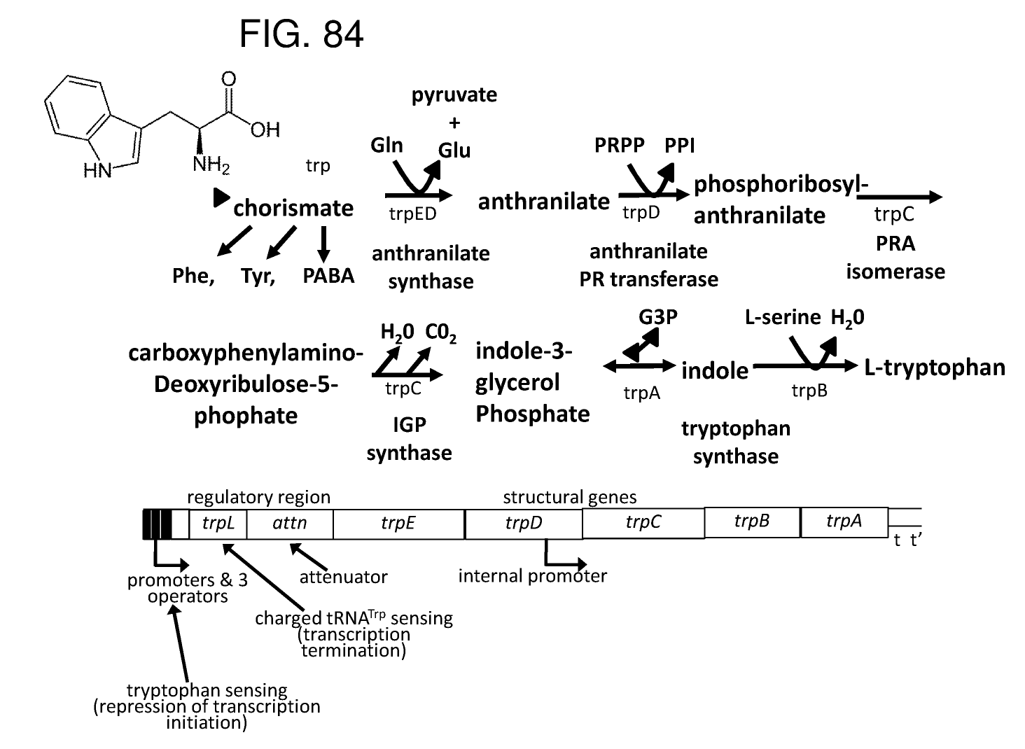

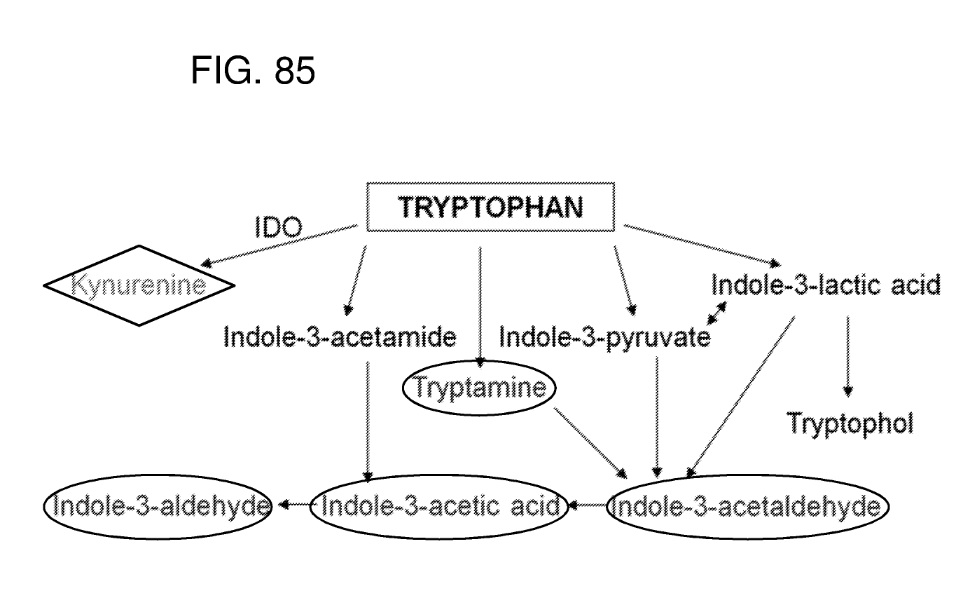

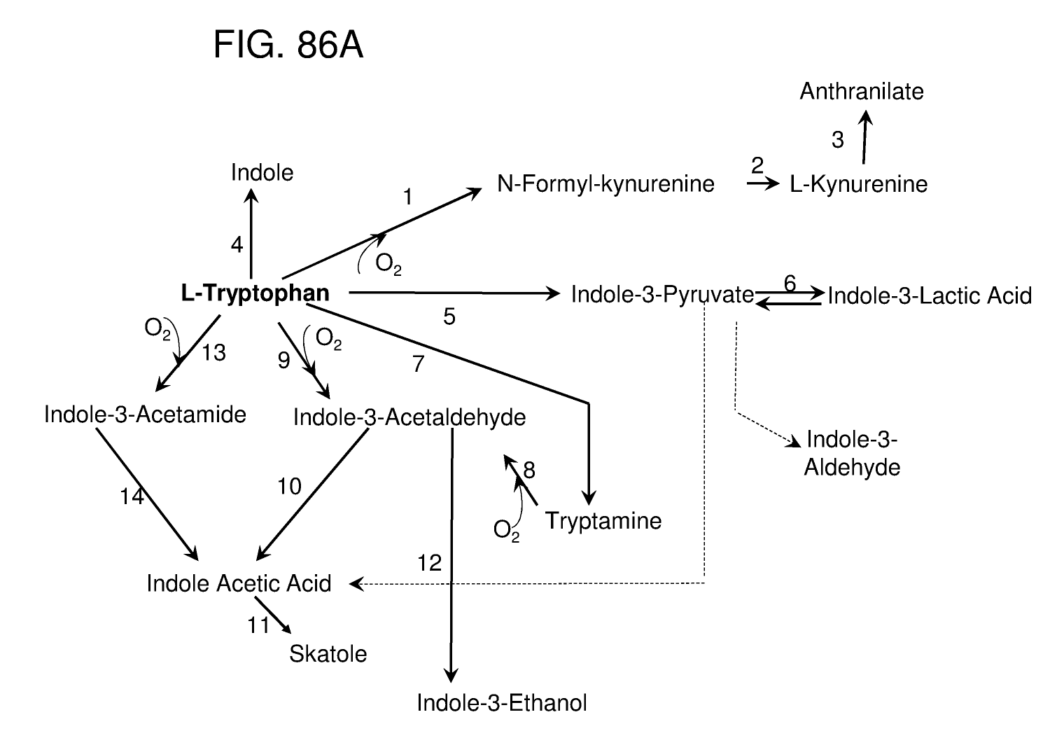

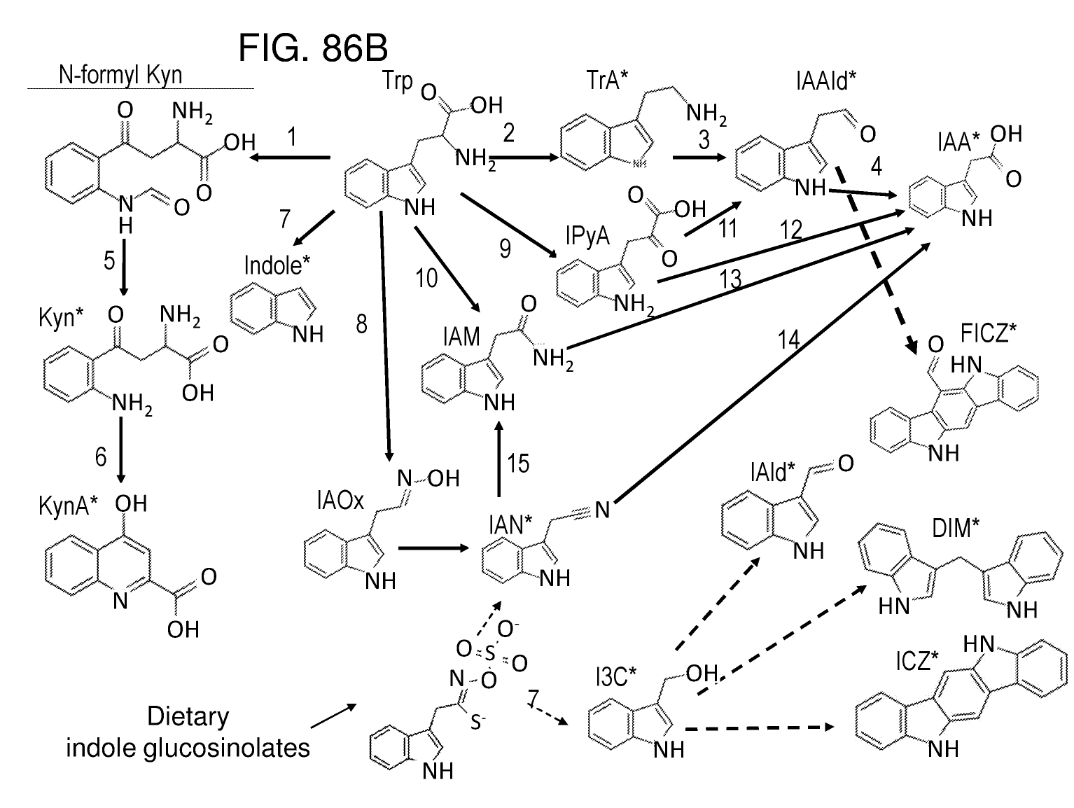

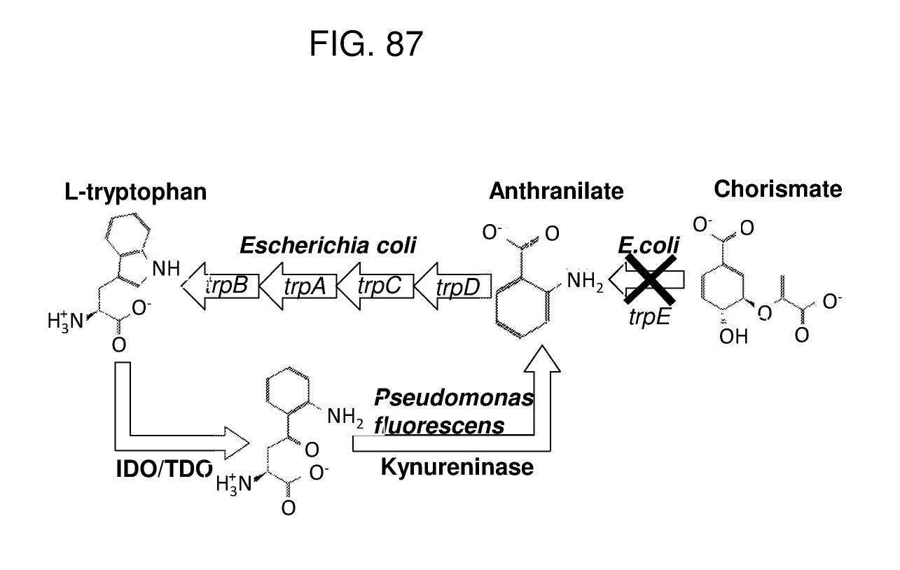

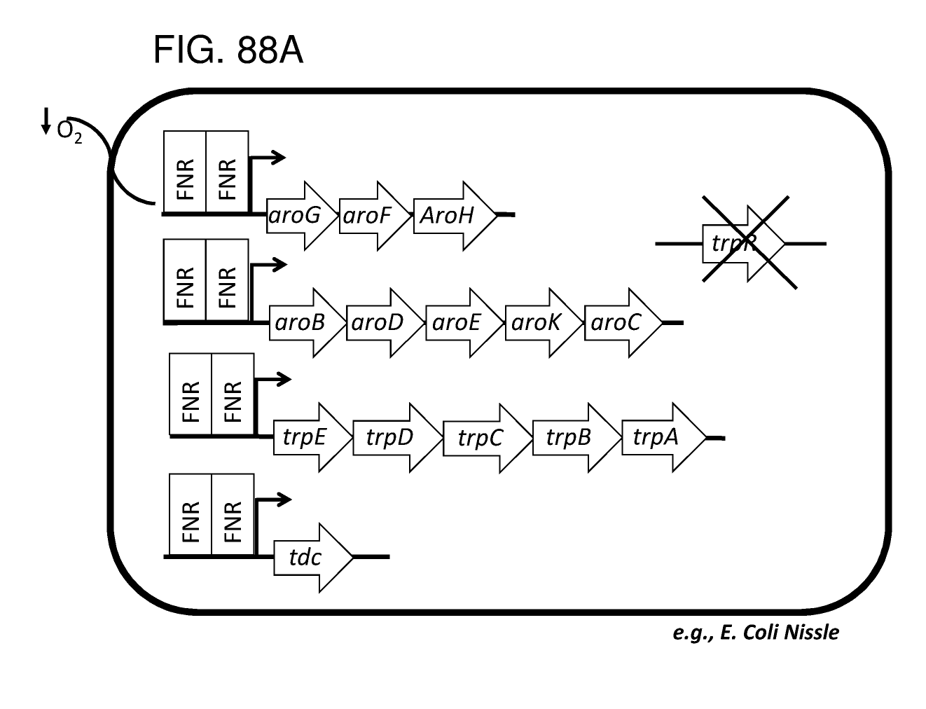

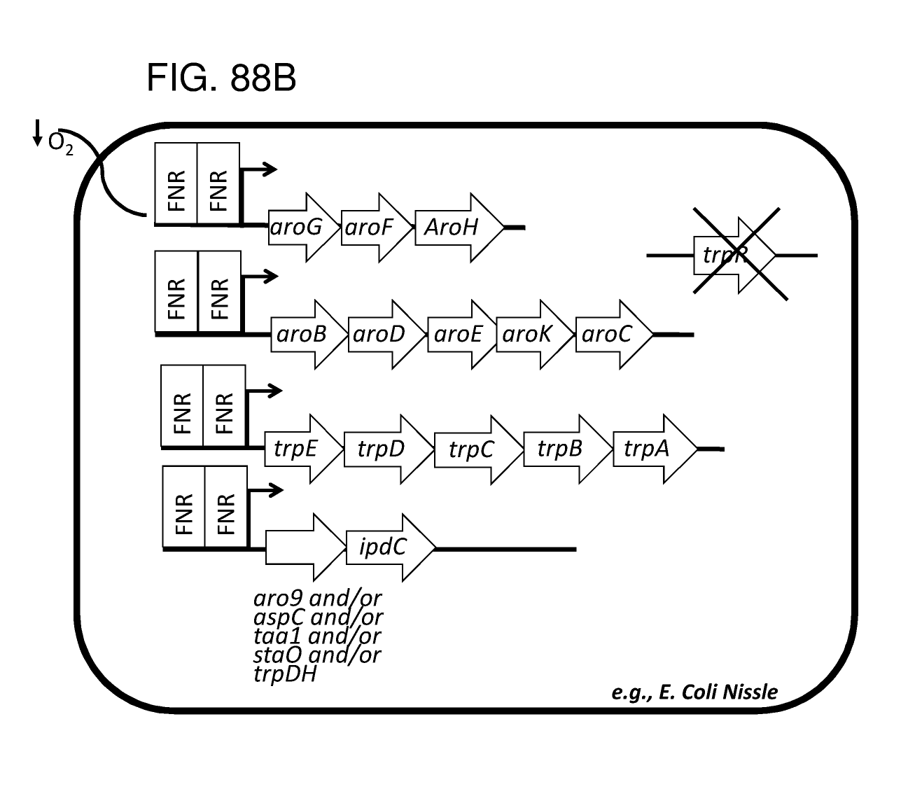

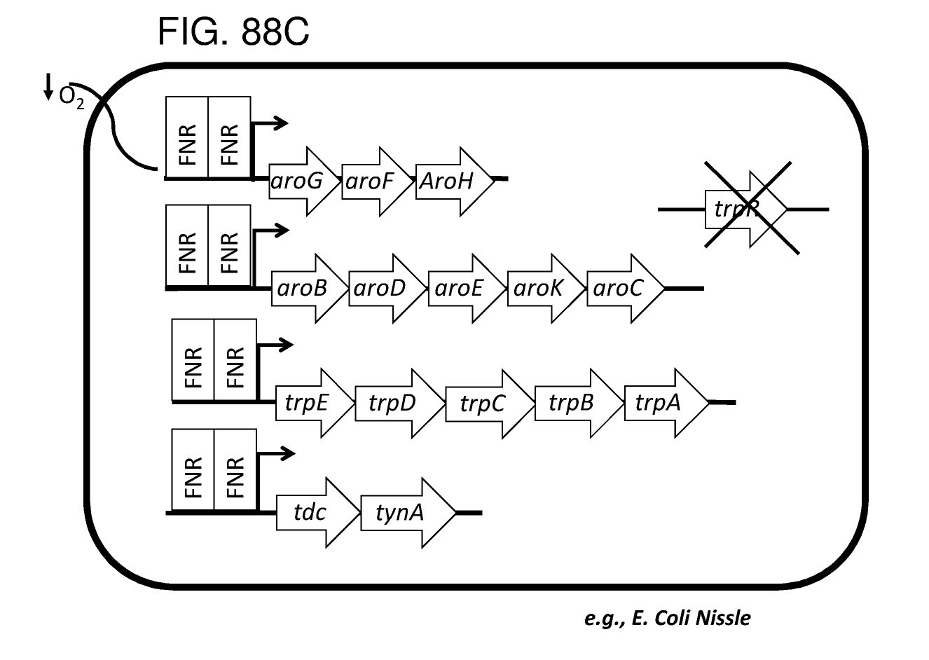

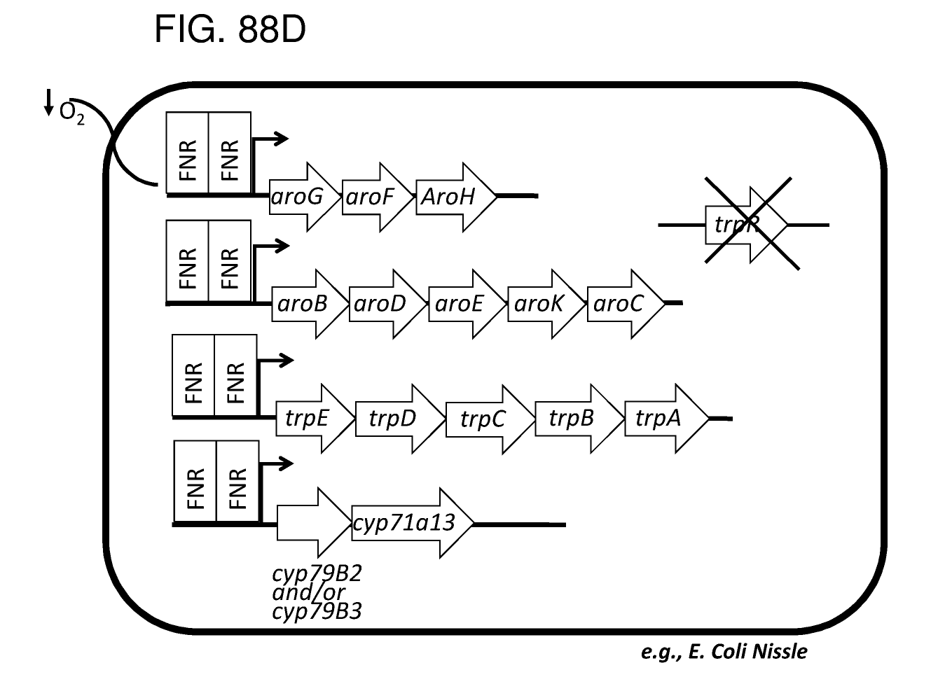

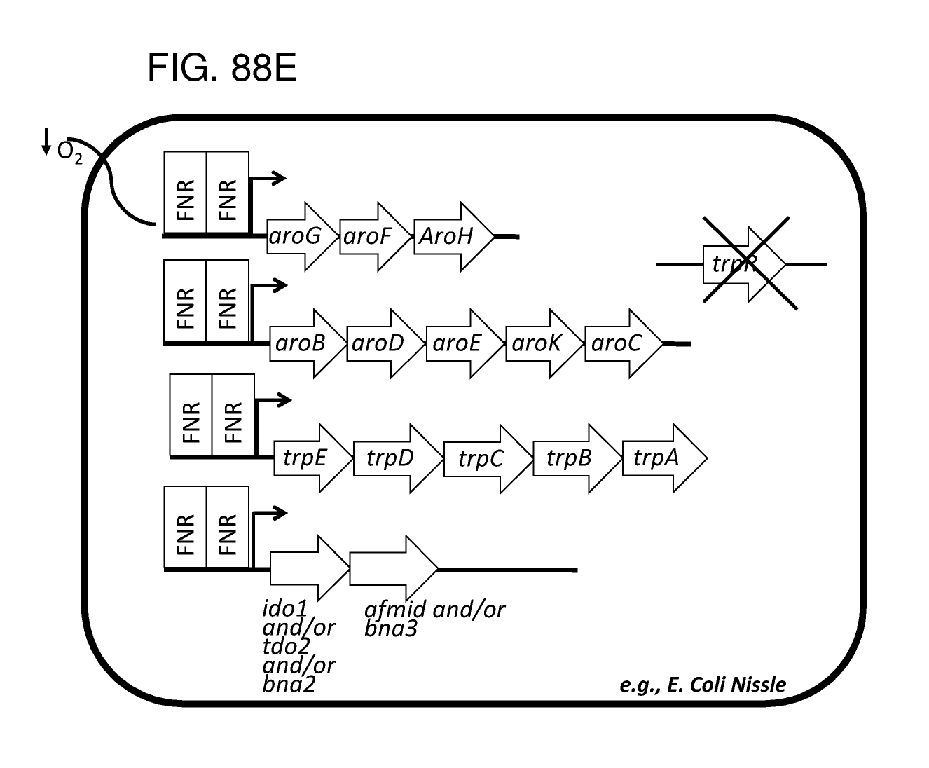

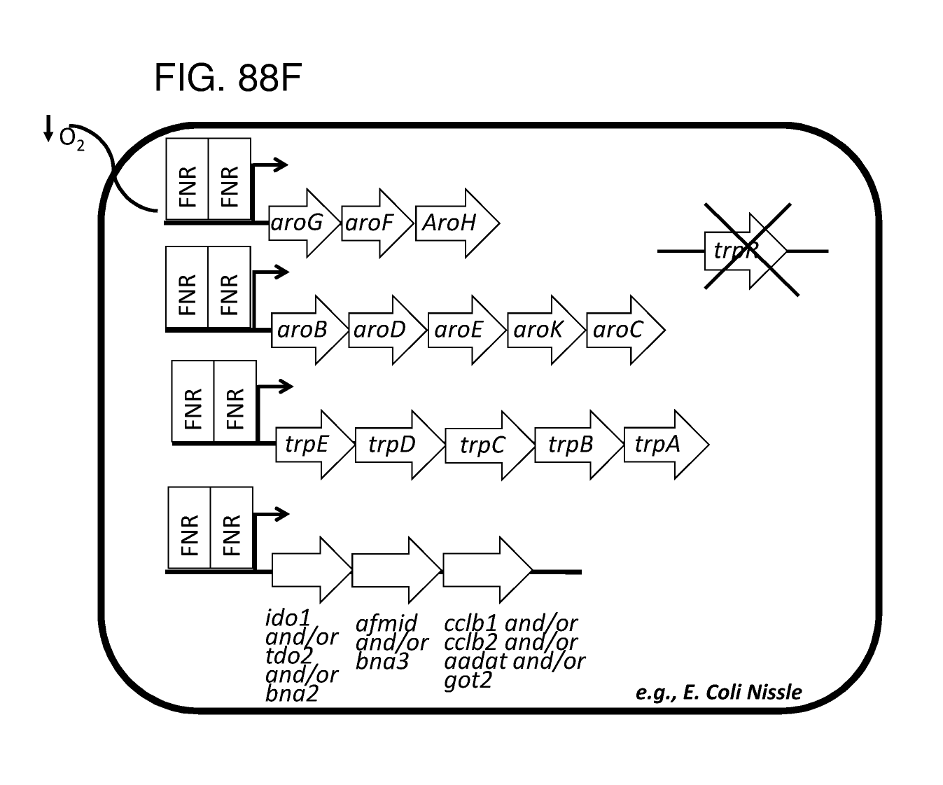

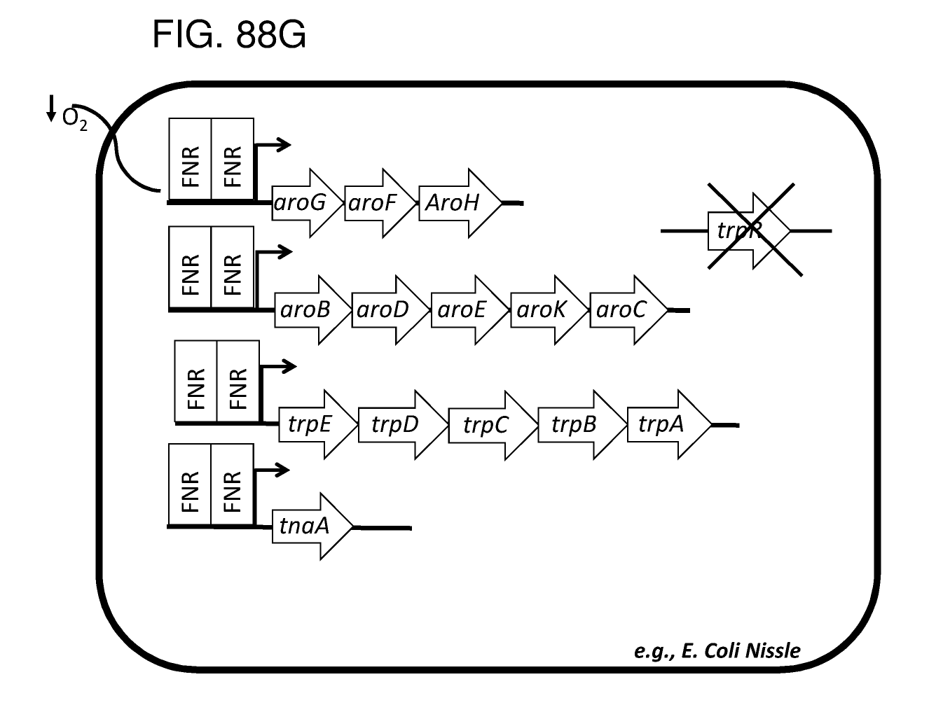

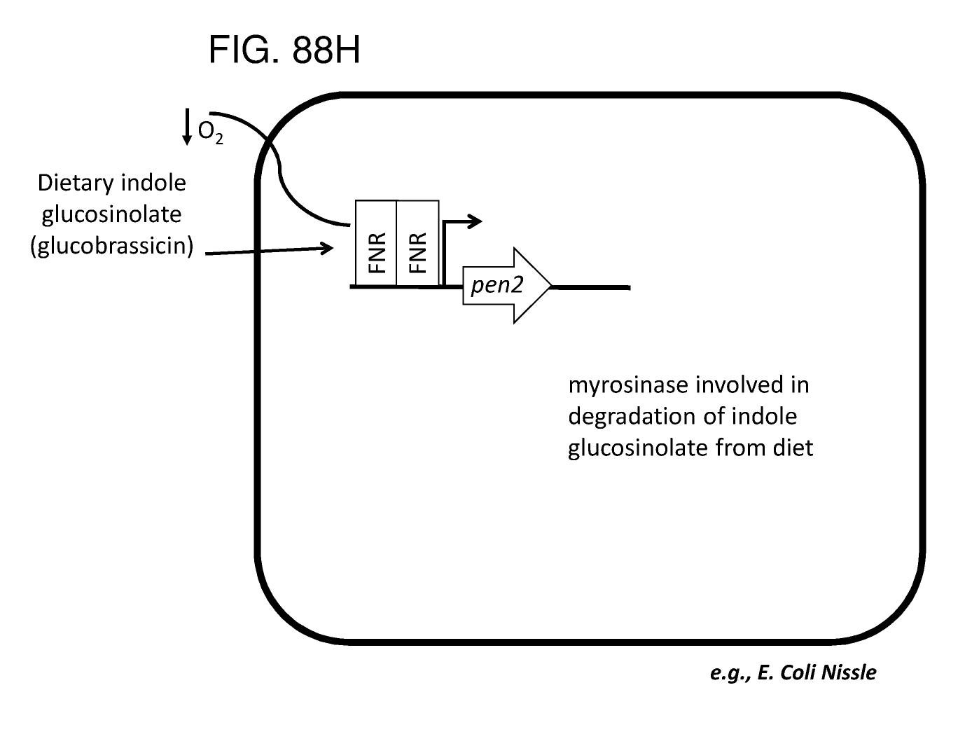

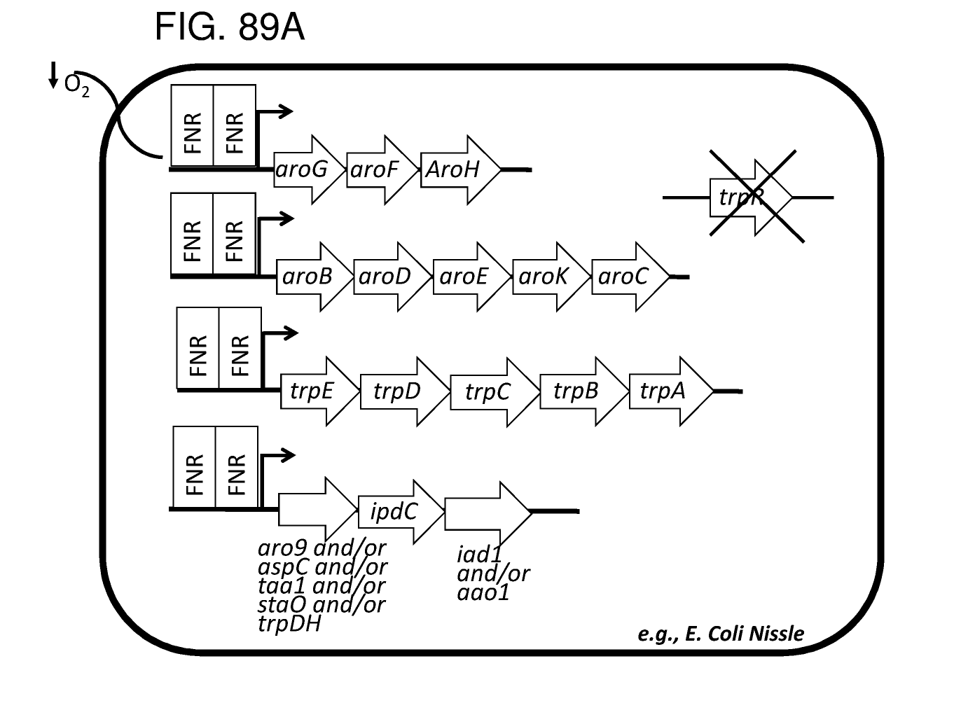

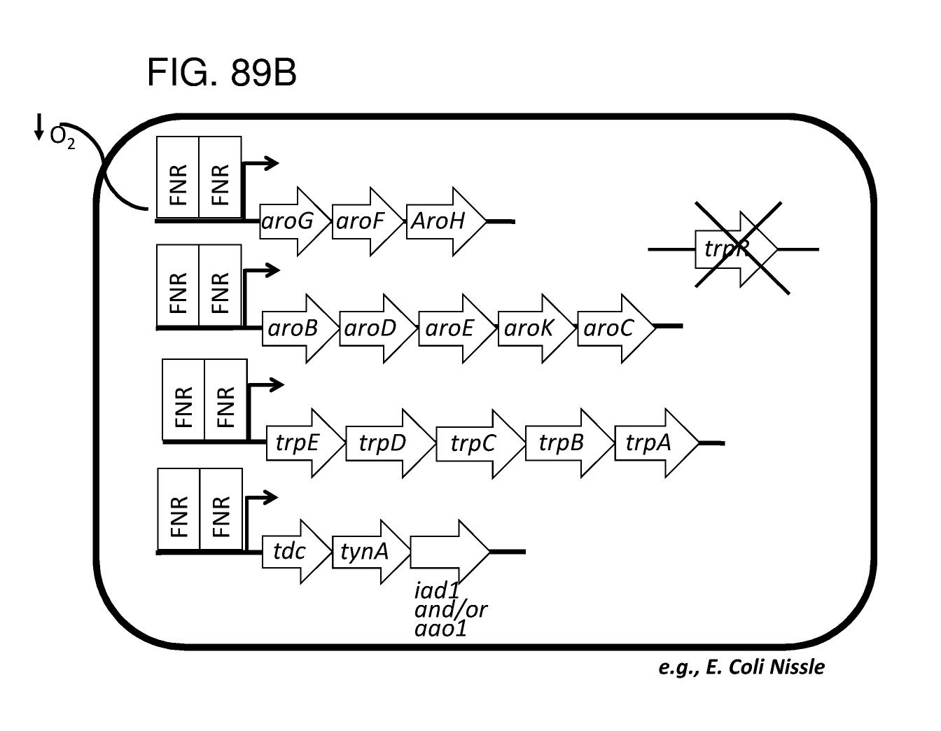

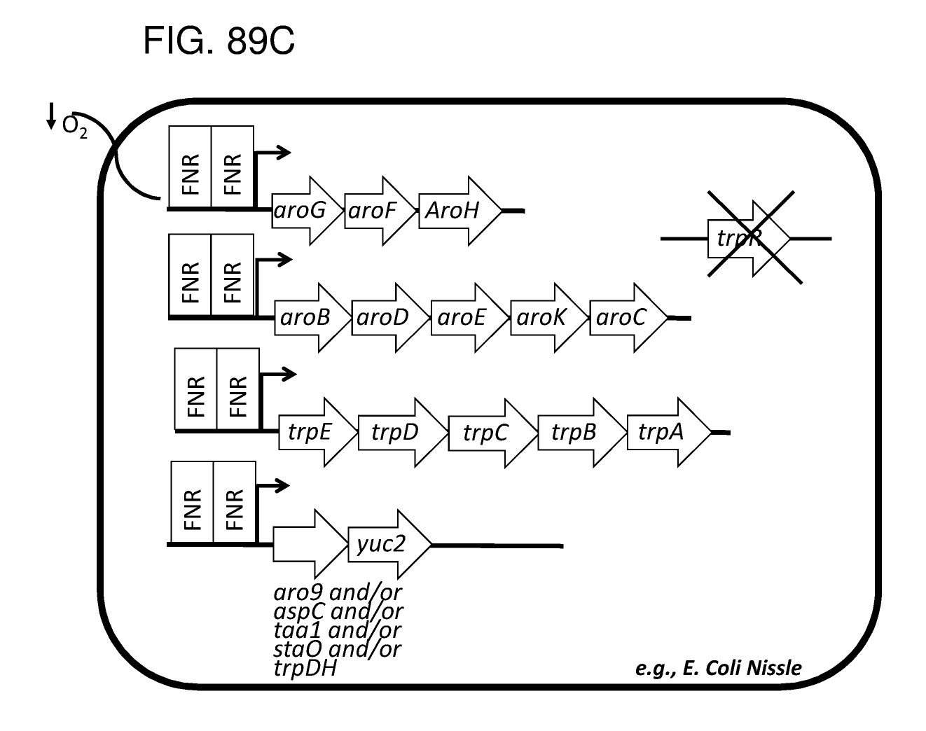

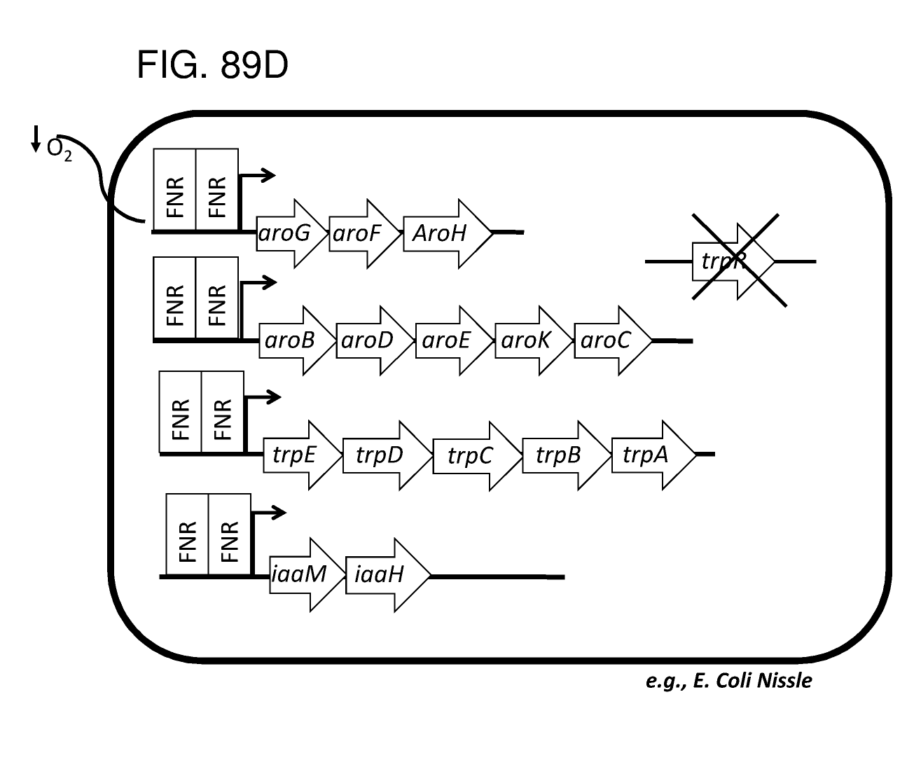

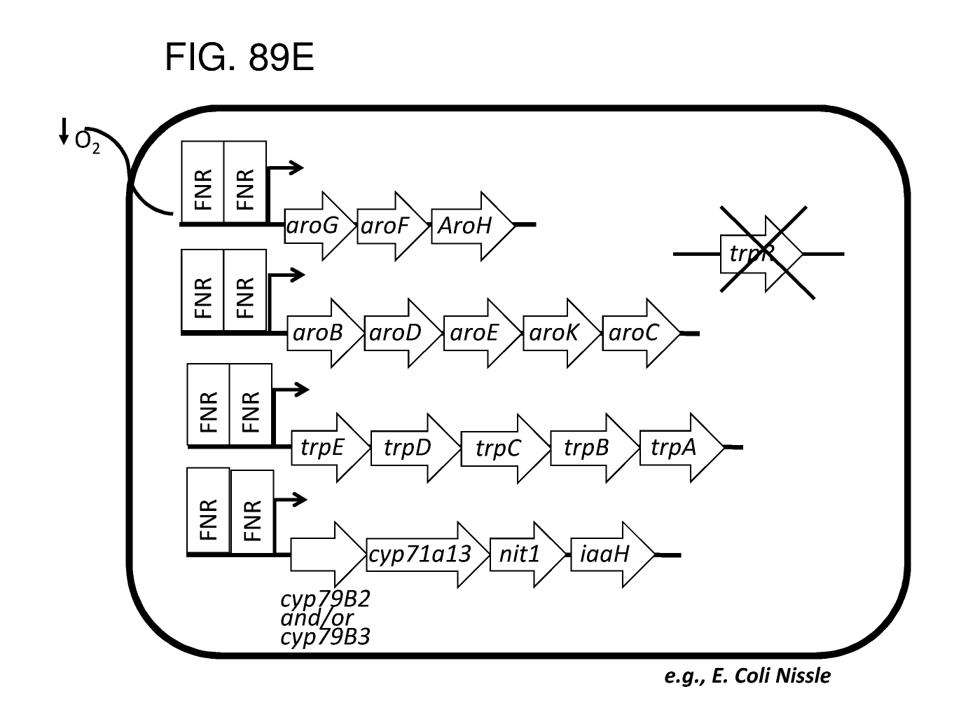

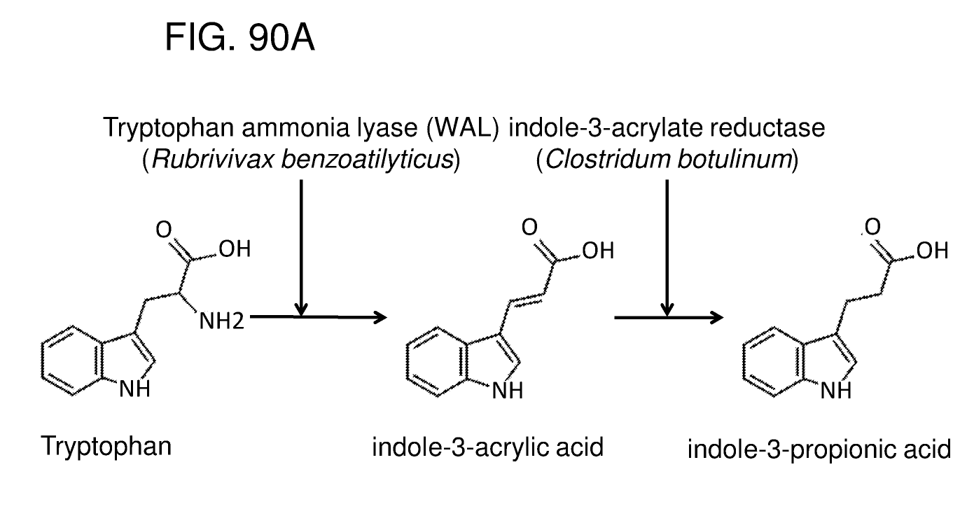

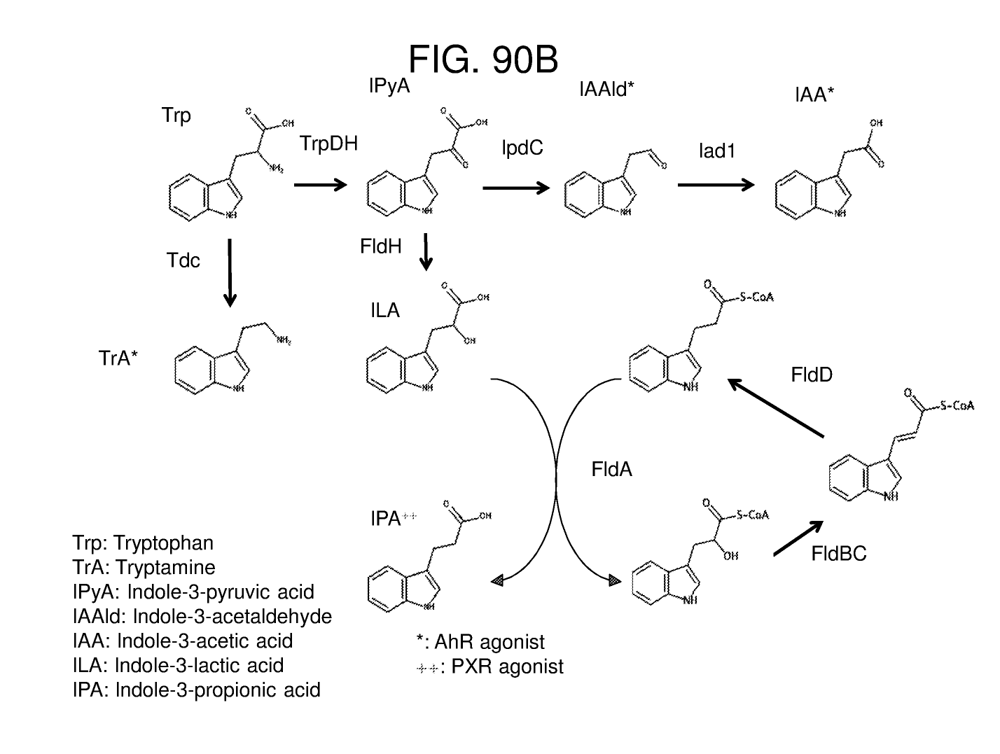

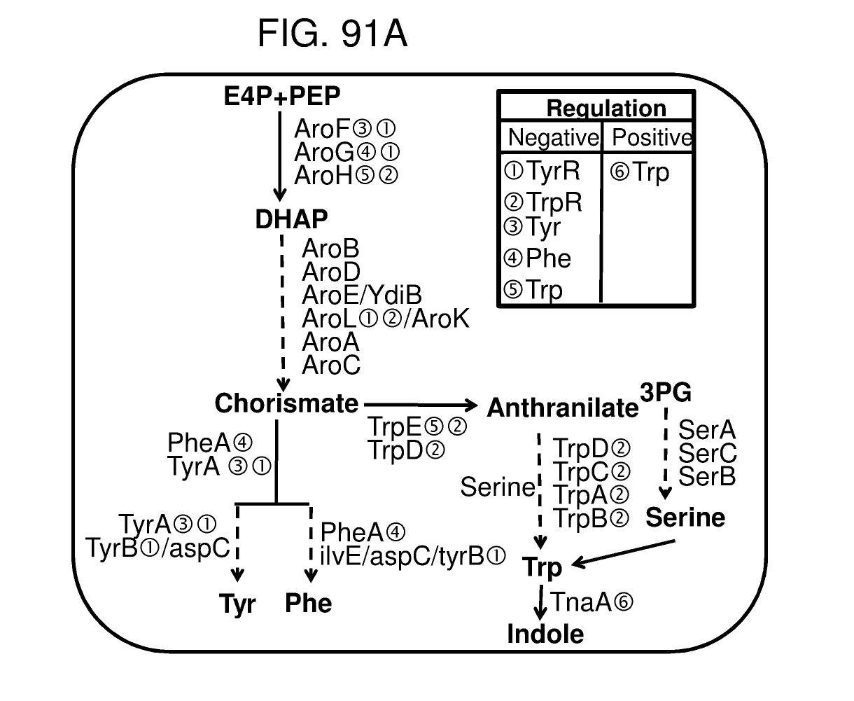

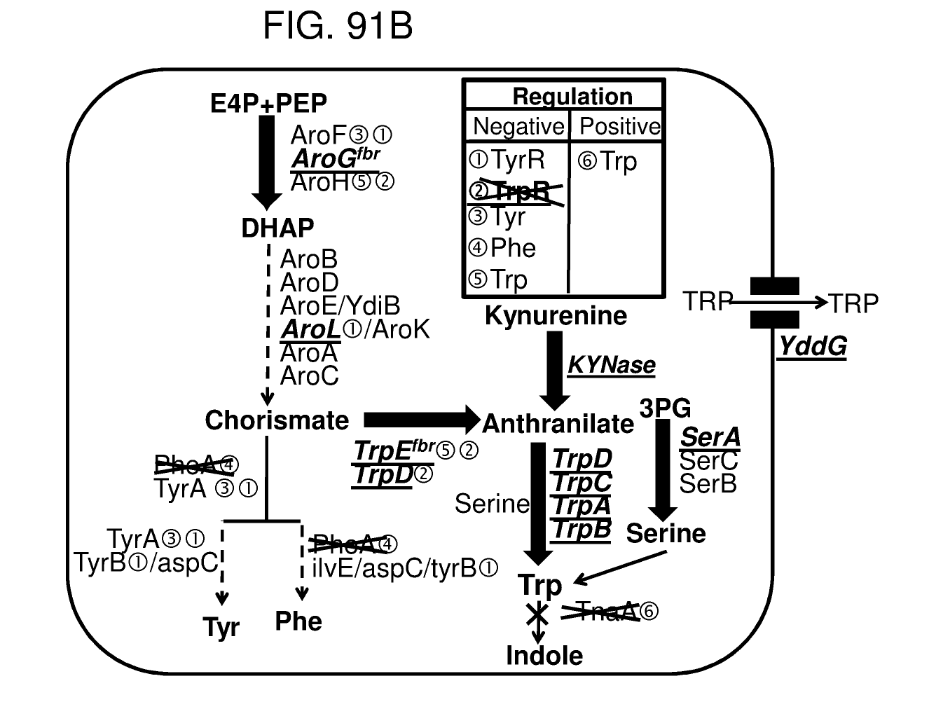

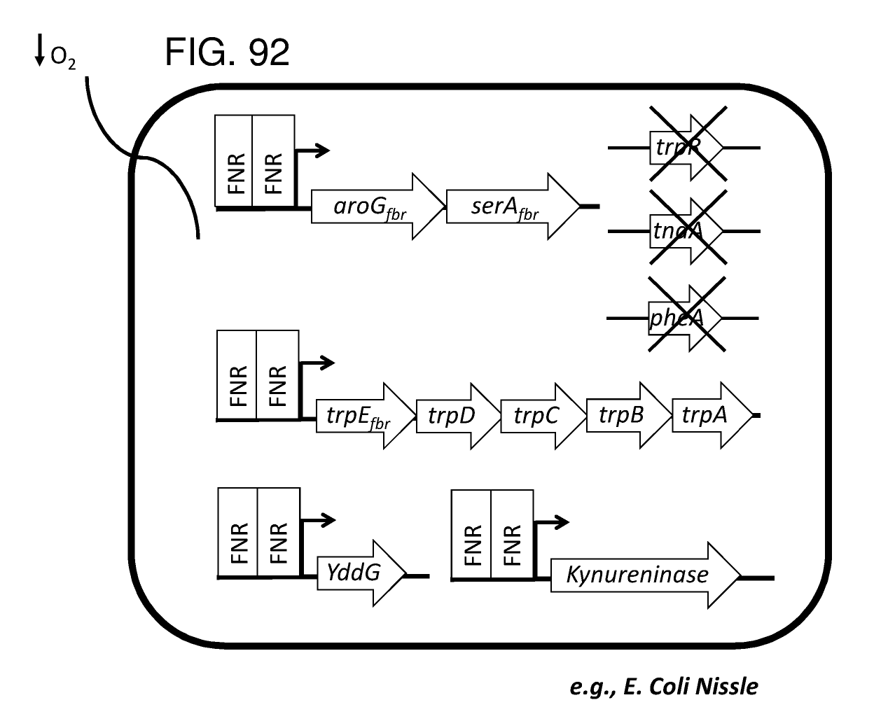

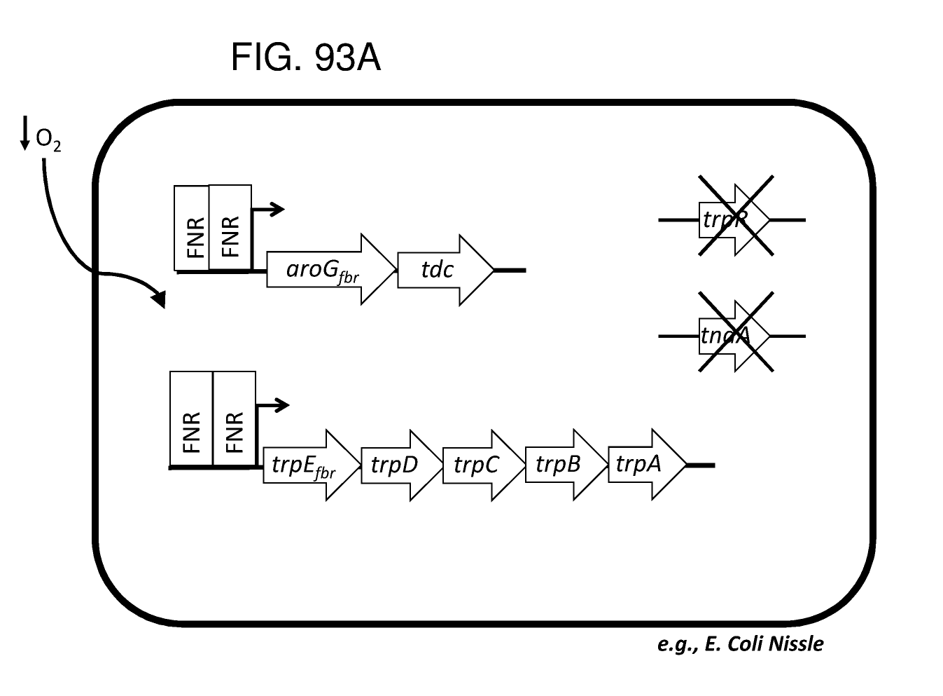

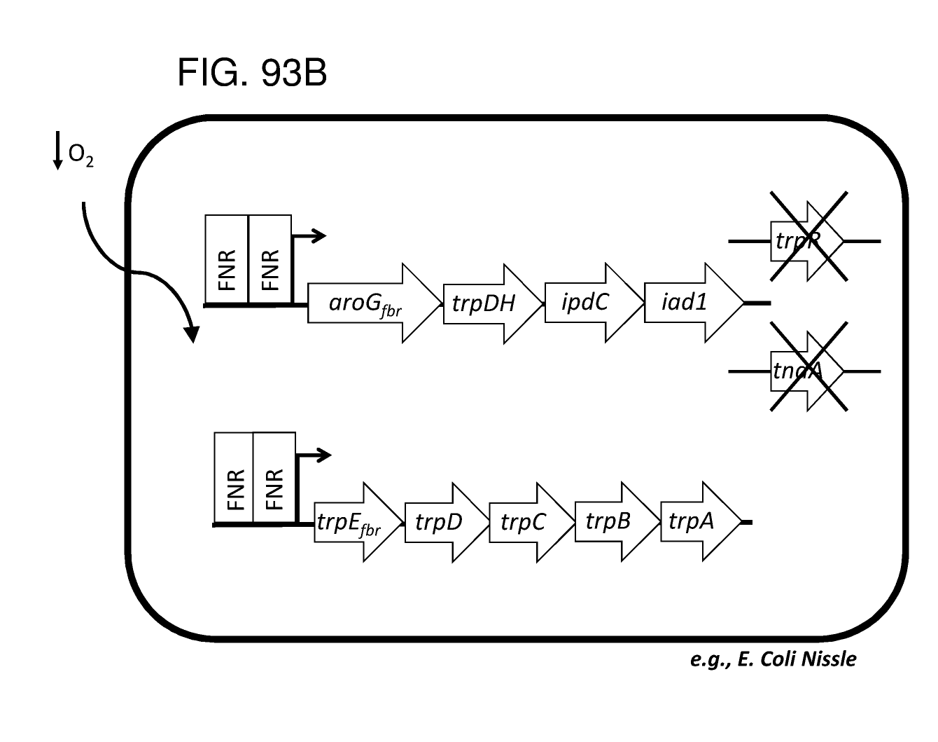

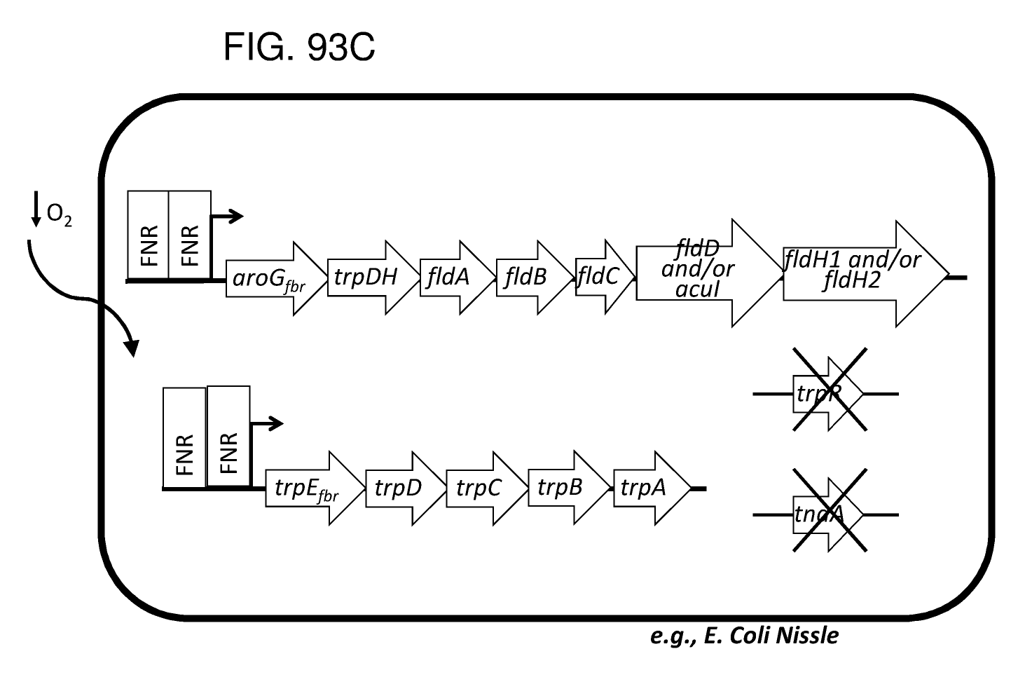

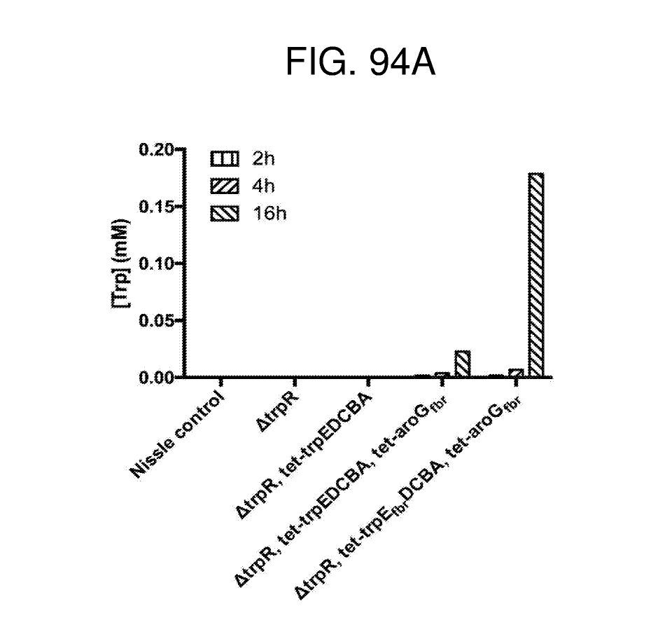

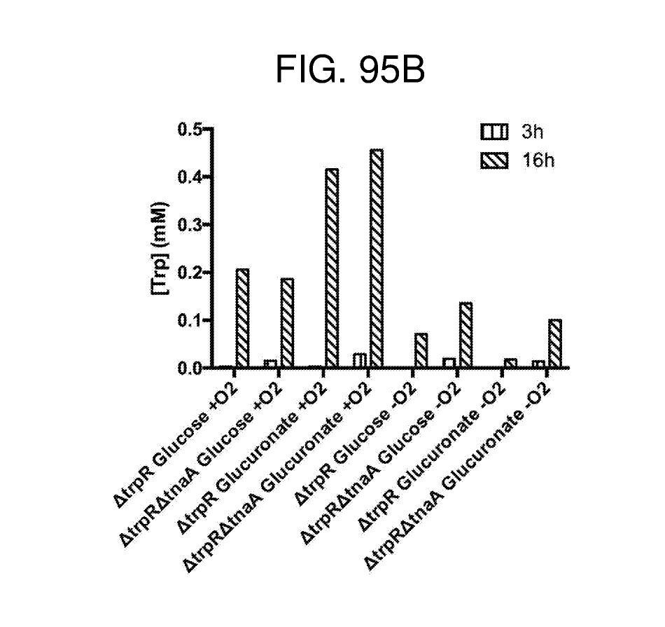

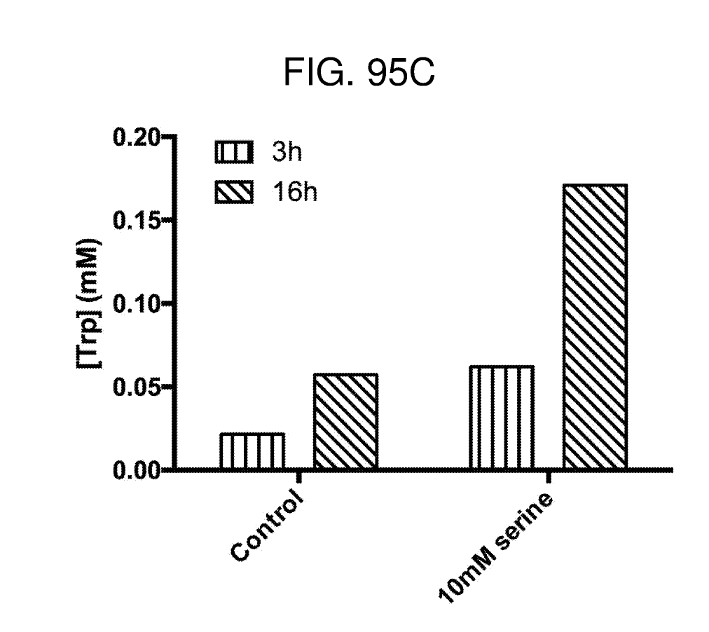

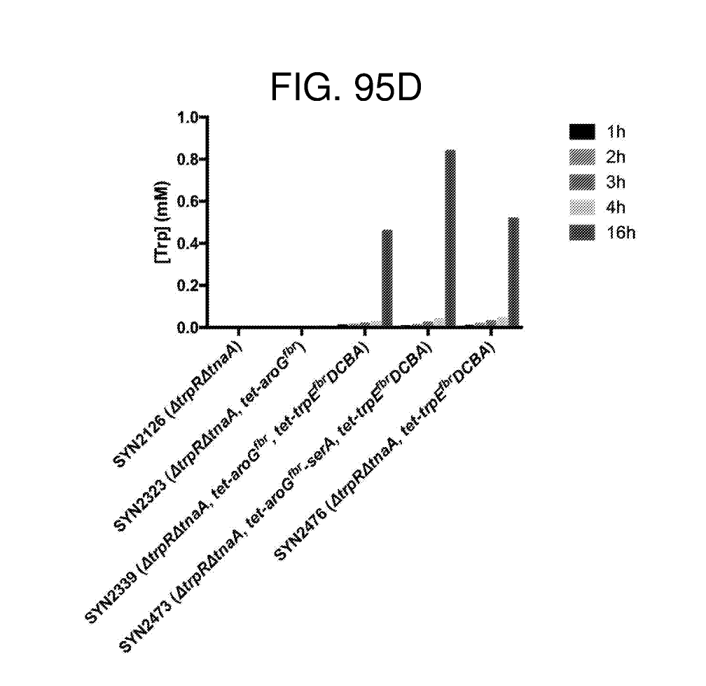

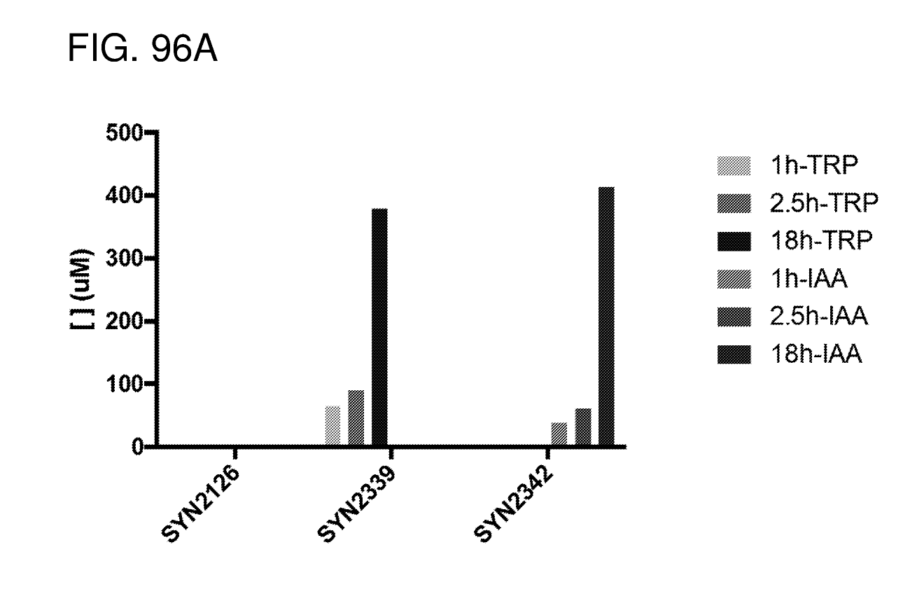

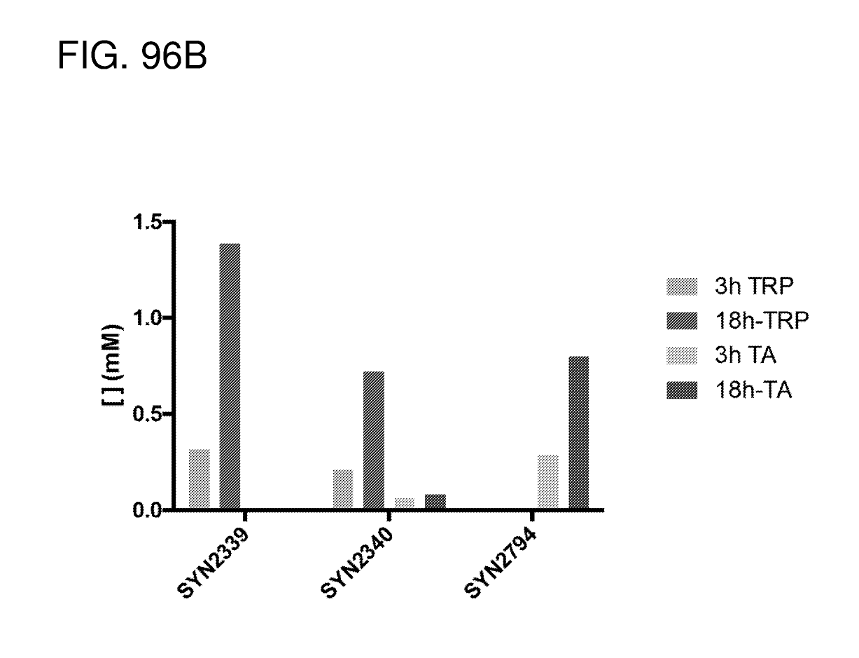

FIG. 82A depicts blood phenylalanine concentrations relative to baseline; and the total reduction in Δphe is listed in Table below.

Decrease in delta[Phe] (%)

| Dose | Decrease in delta[Phe] (%) |
|---|---|
| SYN1967 5.3e10 | 37 |
| SYN1967 1.8e10 | 46 |
| SYN1967 6e9 | 24 |
| SYN1967 2e9 | 26 |

FIG. 82B depicts the urine hippurate concentration at 4 hours post phenylalanine injection. Results in FIG. 82 show that SYN-PKU708 was efficacious in reducing blood phenylalanine and that hippurate was excreted in a dose dependent manner in the cages of mice treated with SYN-PKU708, indicating that the cells were active in vivo.

### Example 56. Activity of Inducible FNRS24Y Expressing Strain SYN-PKU707 Grown Under Aerobic Conditions

The activity of SYN-PKU707 (3XP_{fnrS}-PAL3; 2XP_{fnrS}pheP; Pₐᵣₐ-fnr^{S24Y}), a strain expressing FNRS24Y under the control of the arabinose promoter, was assessed under aerobic growth conditions and compared to the activity achieved under anaerobic conditions.

Overnight cultures of SYN-PKU707, comprising 3XP_{fnrS}-PAL3; 2XP_{fnrS}pheP; Pₐᵣₐ-fnr^{S24Y} were diluted 1:100, and were grown to early log phase for 1.5 h. Cells were grown aerobically for an additional 4 hours in the presence or absence of the inducer arabinose at 0.15% final concentration in 10 ml, 20 ml, or 30 ml flasks. In parallel, in separate samples, the strain was also induced anaerobically for 4 hours in the presence or absence of arabinose. To perform the activity assay, 1e9 cells were resuspended and incubated in assay buffer (M9 media with 0.5% glucose, 50 mM Phe, and 50 mM MOPS). Supernatant samples were taken over time and TCA (the product of PAL) was measured by absorbance at 290 nm to determine the rate of TCA production/PAL activity. As seen in FIG. 51, arabinose-induced expression of fnrS24Y results in high level activity under aerobic conditions in 10 ml, 20 ml, or 30 ml flasks. Additionally, activation in the absence of arabinose under anaerobic conditions is maintained. This indicates that this strain is efficiently pre-induced under aerobic conditions prior to in vivo administration. These results also provide an indication that anaerobic activation without arabinose “in vivo” activation is likely conserved in this strain.

### Example 57. Activity Comparison of SYN2619 vs SYN1967 in the Inducible Acute PKU Model

In vivo activity of two strains, SYN-PKU710 and SYN-PKU708 was compared in the Pah ENU2/2 PKU mouse model. SYN-PKU708, comprises three chromosomal insertions of PAL3 (3× fnrS-PAL (malP/T, yicS/nepl, malE/K)) and two chromosomal copies of pheP (2×fnrS-pheP (lacZ, agaI/rsmI)). SYN-PKU708 further comprises one knocked in copy of the mutated FNR transcription factor FNRS24Y (Para::FNRS24Y) and one copy of LAAD inserted at the same insertion site (the arabinose operon), which is transcribed as a bicistronic message from the endogenous arabinose promoter. SYN-PKU708 further comprises a deltadapA (dapA auxotrophy). SYN-PKU710 comprises three chromosomal insertions of PAL3 (3× fnrS-PAL (malP/T, yicS/nepl, malE/K)) and two chromosomal copies of pheP (2×fnrS-pheP (lacZ, agaI/rsmI)). SYN-PKU710 further comprises two copies of PAL driven by the IPTG inducible Lac-promoter (2×lac-PAL (exo/cea and rhtC/rhtB)) and one copy of the LAAD knocked into the arabinose operon with expression driven by the native Para promoter (Para::LAAD). SYN-PKU710 is a dapA auxotroph.

Beginning 4 days prior to the study (i.e., Days −4-1), Pah ENU2/2 mice (˜11-15 weeks of age) were maintained on phenylalanine-free chow and water that was supplemented with 0.5 grams/L phenylalanine. On day 1, mice were weighed to determine the average weight for each group, and were randomized into treatment groups according to weight as follows: Group 1: H₂O Control (n=12); Group 2: SYN-PKU-710 (n=12); Group3: SYN-PKU-708 (n=12). Blood samples were collected by sub-mandibular skin puncture to determine baseline phenylalanine levels.

Mice were then administered single dose of phenylalanine by subcutaneous injection at 0.1 mg per gram body weight, according to the average group weight. At 1, 2 and 3 h post Phe challenge, the bacteria (or water) were administered to mice by oral gavage (300 ul/dose, total of 3×e10 cfu/mouse administered in three doses). Sodium bicarbonate was added to final concentration of 100 mM for both strains. Urine was collected from all animals up to 4 h post Phe challenge. All treatment groups were bled at 4 h post phenylalanine challenge. Blood and urine was stored on ice for LC/MS analysis. Blood phenylalanine concentrations relative to baseline at 4 hours post SC phenylalanine injection The percentage decrease in dPhe SYN-PKU710 and SYN-PKU708 were calculated to be 29% and 40%, respectively. Total hippurate recovered in urine. Negligible hippurate was recovered in mice treated with dH20.

Cells for this study were prepared in a fermenter as follows. For SYN-PKU708, a freezer vial was thawed and used to inoculate a flask culture of fermentation medium composed of glycerol, yeast extract, soytone, and buffer, and DAP (SYN-PKU708 is deltaDapA). The flask was grown overnight and used to inoculate a fermentation tank containing the same medium at 37° C., pH7, and 60% dissolved oxygen. Following a short initial growth phase, the culture was induced with 0.6 mM arabinose to turn on expression of FNRS24Y to induce FNR-driven PAL and PheP expression. Cells were concentrated by centrifugation and resuspended in a formulation buffer comprising glycerol, sucrose, and buffer to protect the cells during freezing at <−60° C.

For SYN-PKU710, a freezer vial was and used thawed to inoculate a flask culture of fermentation medium composed of glycerol, yeast extract, soytone, buffer, and DAP (SYN-PKU708 is deltaDapA). The flask was grown overnight and used to inoculate a fermentation tank containing the same medium at 37° C., pH7, and 30% dissolved oxygen. Following a short initial growth phase, the culture was induced with 1 mM IPTG to turn on expression of the Plac promoters controlling expression of PAL. LAAD expression was not induced in this study. Following 5 hours of activation, the cells were concentrated by centrifugation and resuspended in a formulation buffer (comprising glycerol, sucrose, and buffer) to protect the cells during freezing at <−60° C.

### Example 58. Assessment of In Vitro and In Vivo Activity of Biosafety System Containing Strain

The activity of the following strains are tested:

SYN-PKU1001 comprises two chromosomal copies of pheP (lacZ::PfnrS-pheP, agaI/rsmI::PfnrS-pheP) and a construct knocked into the dapA locus on the bacterial chromosome (low copy RBS; dapA::constitutive prom1 (BBA_J26100)-Pi(R6K)-constitutive promoter 2(P1)-Kis antitoxin). The strain further comprises a plasmid, except that the bla gene is replaced with the construct of (Lad Fnrs-Ptac-PAL-PAL)

SYN-PKU1002 comprises two chromosomal copies of pheP (lacZ::PfnrS-pheP, agaI/rsmI::PfnrS-pheP) and a construct knocked into the dapA locus on the bacterial chromosome (low copy RBS; dapA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

SYN-PKU1003 comprises two chromosomal copies of pheP (lacZ::PfnrS-pheP, agaI/rsmI::PfnrS-pheP) and a construct knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid, except that the bla gene is replaced with the construct of (Lad Fnrs-Ptac-PAL-PAL).

SYN-PKU1004 comprises two chromosomal copies of pheP (lacZ::PfnrS-pheP, agaI/rsmI::PfnrS-pheP) and a construct knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

SYN-PKU1005 comprises two chromosomal copies of pheP (lacZ::PfnrS-pheP, agaI/rsmI::PfnrS-pheP) and a construct knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid, except that the bla gene is replaced with the construct of Lad Fnrs-Ptac-PAL-PAL)

SYN-PKU1006 comprises two chromosomal copies of pheP (lacZ::PfnrS-pheP, agaI/rsmI::PfnrS-pheP) and a construct knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

SYN-PKU1007 comprises two chromosomal copies of pheP (lacZ::PfnrS-pheP, agaI/rsmI::PfnrS-pheP) and a construct knocked into the dapA locus on the bacterial chromosome (medium copy RBS; thyA::constitutive prom1 (BBA_J26100)-Pi(R6K)-constitutive promoter 2(P1)-Kis antitoxin). The strain further comprises a plasmid, except that the bla gene is replaced with the construct of (Lad Fnrs-Ptac-PAL-PAL).

SYN-PKU1008 comprises two chromosomal copies of pheP (lacZ::PfnrS-pheP, agaI/rsmI::PfnrS-pheP) and a construct knocked into the thyA locus on the bacterial chromosome (medium copy RBS; thyA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

SYN-PKU1009 a construct knocked into the dapA locus on the bacterial chromosome (low copy RBS; dapA::constitutive prom1 (BBA_J26100)-Pi(R6K)-constitutive promoter 2(P1)-Kis antitoxin). The strain further comprises a plasmid, except that the bla gene is replaced with the construct of (Lad Fnrs-Ptac-PAL-PAL)

SYN-PKU1010 a construct knocked into the dapA locus on the bacterial chromosome (low copy RBS;dapA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

SYN-PKU1011 comprises a construct knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid, except that the bla gene is replaced with the construct of (Lad Fnrs-Ptac-PAL-PAL).

SYN-PKU1012 a construct knocked into the dapA locus on the bacterial chromosome (medium copy RBS; dapA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

SYN-PKU1013 comprises a construct knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid, except that the bla gene is replaced with the construct of (Lad Fnrs-Ptac-PAL-PAL)

SYN-PKU1014 comprises a construct knocked into the thyA locus on the bacterial chromosome (low copy RBS; thyA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

SYN-PKU1015 comprises a construct knocked into the dapA locus on the bacterial chromosome (medium copy RBS; thyA::constitutive prom1 (BBA_J26100)-Pi(R6K)-constitutive promoter 2(P1)-Kis antitoxin). The strain further comprises a plasmid, except that the bla gene is replaced with the construct of (Lad Fnrs-Ptac-PAL-PAL).

SYN-PKU1016 knocked into the thyA locus on the bacterial chromosome (medium copy RBS; thyA::constitutive prom1 (BBA_J26100)-Pi(R6K)-constitutive promoter 2(P1)-Kis antitoxin). The strain further comprises a plasmid, except that the bla gene is replaced with the construct of (lacI-Ptac-PAL-PAL).

Cells are grown overnight in LB and diluted 1:100. After 1.5 hrs of growth, Cells are grown for 4 hours in the presence of 1 mM IPTG 1 mM to turn on expression of the Plac promoters controlling expression of PAL (and in some cases PheP). Bacteria are spun down and are resuspended in assay buffer containing 50 mM phenylalanine. Aliquots are removed from cell assays every 20 min for 1.5 hrs for trans-cinnamate quantification by absorbance at 290 nm. In another study, the same constructs used above are employed, with the strain further comprising chromosomally integrated Para-LAAD, which is induced in parallel with PLacI. In another study the same constructs as above are employed except that the strains further comprise chromosomally integrated Para-FNRS24Y. In another study the same constructs as above are employed except that the strains further comprise chromosomally integrated Para-FNRS24Y-LAAD.

In some embodiments, the PAL3 used in the above strains is codon optimized. In other embodiments, the original PAL3 sequence from Photorhabdus chemiluminescens as described herein is used in any of the constructs described above.

### Example 59. Strain Activity

The activity of SYN-707, SYN-PKU710, and SYN-PKU708 was measured in vitro. Results are reported in Table 83.

For SYN-PKU710, cells were grown to OD 0.2 in fermentation media and induced by addition of 1 mM IPTG for one hour. Then, was added arabinose at 0.009% final concentration. Cells were induced for another for 4 hours. For SYN-PKU708 and SYN-PKU707, cells were grown to OD 0.2 in fermentation media and induced by addition of arabinose at 0.15% final concentration for 4 hours. In the presence of oxygen (shaking), high levels of activity are observed which are not limited by oxygen, glucose, pH or substrate. Under microaerobic conditions (static incubation), LAAD activity is dependent on oxygen. Results are reported in Table below.

### Example 60. Generation of E. Coli Mutants with Ability to Consume L-Kynurenine and Produce Tryptophan from Kynurenine

E. coli Nissle can be engineered to efficiently import KYN and convert it to TRP. A strain was constructed with a knock out in TrpE (tryptophan auxotroph) that also expresses exogenous Pseudomonas fluorescens kynureninase on a medium copy plasmid and driven by a tetracycline inducible promoter. In the presence of tetracycline, this strain is capable of converting L-kynurenine to anthranilate. Anthranilate can then be converted tryptophan through the enzymes of the tryptophan biosynthetic pathway.

Table of Constructs for Tet-inducible Expression of
Pseudomonas fluorescens Kynureninase

| | | SEQ ID |
|---|---|---|
| Description | Sequence | NO |
| Pseudomonas | ATGACGACCCGAAATGATTGCCTAGCGTTGGAT | |
| fluorescens | GCACAGGACAGTCTGGCTCCGCTGCGCCAACAA | |
| kynureninase, codon | TTTGCGCTGCCGGAGGGTGTGATATACCTGGAT | |
| optimized for | GGCAATTCGCTGGGCGCACGTCCGGTAGCTGCG | |
| expression in E. coli | CTGGCTCGCGCGCAGGCTGTGATCGCAGAAGA | |
| SEQ ID NO: | ATGGGGCAACGGGTTGATCCGTTCATGGAACTC | |
| | TGCGGGCTGGCGTGATCTGTCTGAACGCCTGGG | |
| | TAATCGCCTGGCTACCCTGATTGGTGCGCGCGA | |
| | TGGGGAAGTAGTTGTTACTGATACCACCTCGAT | |
| | TAATCTGTTTAAAGTGCTGTCAGCGGCGCTGCG | |
| | CGTGCAAGCTACCCGTAGCCCGGAGCGCCGTGT | |
| | TATCGTGACTGAGACCTCGAATTTCCCGACCGA | |
| | CCTGTATATTGCGGAAGGGTTGGCGGATATGCT | |
| | GCAACAAGGTTACACTCTGCGTTTGGTGGATTC | |
| | ACCGGAAGAGCTGCCACAGGCTATAGATCAGG | |
| | ACACCGCGGTGGTGATGCTGACGCACGTAAATT | |
| | ATAAAACCGGTTATATGCACGACATGCAGGCTC | |
| | TGACCGCGTTGAGCCACGAGTGTGGGGCTCTGG | |
| | CGATTTGGGATCTGGCGCACTCTGCTGGCGCTG | |
| | TGCCGGTGGACCTGCACCAAGCGGGCGCGGAC | |
| | TATGCGATTGGCTGCACGTACAAATACCTGAAT | |
| | GGCGGCCCGGGTTCGCAAGCGTTTGTTTGGGTT | |
| | TCGCCGCAACTGTGCGACCTGGTACCGCAGCCG | |
| | CTGTCTGGTTGGTTCGGCCATAGTCGCCAATTC | |
| | GCGATGGAGCCGCGCTACGAACCTTCTAACGGC | |
| | ATTGCTCGCTATCTGTGCGGCACTCAGCCTATT | |
| | ACTAGCTTGGCTATGGTGGAGTGCGGCCTGGAT | |
| | GTGTTTGCGCAGACGGATATGGCTTCGCTGCGC | |
| | CGTAAAAGTCTGGCGCTGACTGATCTGTTCATC | |
| | GAGCTGGTTGAACAACGCTGCGCTGCACACGA | |
| | ACTGACCCTGGTTACTCCACGTGAACACGCGAA | |
| | ACGCGGCTCTCACGTGTCTTTTGAACACCCCGA | |
| | GGGTTACGCTGTTATTCAAGCTCTGATTGATCG | |
| | TGGCGTGATCGGCGATTACCGTGAGCCACGTAT | |
| | TATGCGTTTCGGTTTCACTCCTCTGTATACTACT | |
| | TTTACGGAAGTTTGGGATGCAGTACAAATCCTG | |
| | GGCGAAATCCTGGATCGTAAGACTTGGGCGCA | |
| | GGCTCAGTTTCAGGTGCGCCACTCTGTTACTTAA | |
| Pseudomonas | TAATTCCTAATTTTTGTTGACACTCTATCATTGA | |
| fluorescens | TAGAGTTATTTTACCACTCCCTATCAGTGATAG | |
| kynureninase, codon | AGAAAAGTGAATTATATAAAAGTGGGAGGTGC | |
| optimized for | CCGAATGACGACCCGAAATGATTGCCTAGCGTT | |
| expression in E. coli | GGATGCACAGGACAGTCTGGCTCCGCTGCGCCA | |
| driven by a Tet | ACAATTTGCGCTGCCGGAGGGTGTGATATACCT | |
| inducible promoter | GGATGGCAATTCGCTGGGCGCACGTCCGGTAGC | |
| SEQ ID NO: | TGCGCTGGCTCGCGCGCAGGCTGTGATCGCAGA | |
| | AGAATGGGGCAACGGGTTGATCCGTTCATGGA | |
| | ACTCTGCGGGCTGGCGTGATCTGTCTGAACGCC | |
| | TGGGTAATCGCCTGGCTACCCTGATTGGTGCGC | |
| | GCGATGGGGAAGTAGTTGTTACTGATACCACCT | |
| | CGATTAATCTGTTTAAAGTGCTGTCAGCGGCGC | |
| | TGCGCGTGCAAGCTACCCGTAGCCCGGAGCGCC | |
| | GTGTTATCGTGACTGAGACCTCGAATTTCCCGA | |
| | CCGACCTGTATATTGCGGAAGGGTTGGCGGATA | |
| | TGCTGCAACAAGGTTACACTCTGCGTTTGGTGG | |
| | ATTCACCGGAAGAGCTGCCACAGGCTATAGATC | |
| | AGGACACCGCGGTGGTGATGCTGACGCACGTA | |
| | AATTATAAAACCGGTTATATGCACGACATGCAG | |
| | GCTCTGACCGCGTTGAGCCACGAGTGTGGGGCT | |
| | CTGGCGATTTGGGATCTGGCGCACTCTGCTGGC | |
| | GCTGTGCCGGTGGACCTGCACCAAGCGGGCGC | |
| | GGACTATGCGATTGGCTGCACGTACAAATACCT | |
| | GAATGGCGGCCCGGGTTCGCAAGCGTTTGTTTG | |
| | GGTTTCGCCGCAACTGTGCGACCTGGTACCGCA | |
| | GCCGCTGTCTGGTTGGTTCGGCCATAGTCGCCA | |
| | ATTCGCGATGGAGCCGCGCTACGAACCTTCTAA | |
| | CGGCATTGCTCGCTATCTGTGCGGCACTCAGCC | |
| | TATTACTAGCTTGGCTATGGTGGAGTGCGGCCT | |
| | GGATGTGTTTGCGCAGACGGATATGGCTTCGCT | |
| | GCGCCGTAAAAGTCTGGCGCTGACTGATCTGTT | |
| | CATCGAGCTGGTTGAACAACGCTGCGCTGCACA | |
| | CGAACTGACCCTGGTTACTCCACGTGAACACGC | |
| | GAAACGCGGCTCTCACGTGTCTTTTGAACACCC | |
| | CGAGGGTTACGCTGTTATTCAAGCTCTGATTGA | |
| | TCGTGGCGTGATCGGCGATTACCGTGAGCCACG | |
| | TATTATGCGTTTCGGTTTCACTCCTCTGTATACT | |
| | ACTTTTACGGAAGTTTGGGATGCAGTACAAATC | |
| | CTGGGCGAAATCCTGGATCGTAAGACTTGGGCG | |
| | CAGGCTCAGTTTCAGGTGCGCCACTCTGTTACT | |
| | TAA | |
| Pseudomonas | TTAAGACCCACTTTCACATTTAAGTTGTTTTTCT | |
| fluorescens | AATCCGCATATGATCAATTCAAGGCCGAATAAG | |
| kynureninase, codon | AAGGCTGGCTCTGCACCTTGGTGATCAAATAAT | |
| optimized for | TCGATAGCTTGTCGTAATAATGGCGGCATACTA | |
| expression in E. coli | TCAGTAGTAGGTGTTTCCCTTTCTTCTTTAGCGA | |
| (no underline, no | CTTGATGCTCTTGATCTTCCAATACGCAACCTA | |
| italic) driven by a Tet | AAGTAAAATGCCCCACAGCGCTGAGTGCATATA | |
| inducible promoter | ATGCATTCTCTAGTGAAAAACCTTGTTGGCATA | |
| (underlined italic) | AAAAGGCTAATTGATTTTCGAGAGTTTCATACT | |
| with RBS (italic, and | GTTTTTCTGTAGGCCGTGTACCTAAATGTACTTT | |
| tetR in reverse | TGCTCCATCGCGATGACTTAGTAAAGCACATCT | |
| orientation | AAAACTTTTAGCGTTATTACGTAAAAAATCTTG | |
| (underlined) | CCAGCTTTCCCCTTCTAAAGGGCAAAAGTGAGT | |
| SEQ ID NO: XXX | ATGGTGCCTATCTAACATCTCAATGGCTAAGGC | |
| | GTCGAGCAAAGCCCGCTTATTTTTTACATGCCA | |
| | ATACAATGTAGGCTGCTCTACACCTAGCTTCTG | |
| | GGCGAGTTTACGGGTTGTTAAACCTTCGATTCC | |
| | GACCTCATTAAGCAGCTCTAATGCGCTGTTAAT | |
| | CACTTTACTTTTATCTAATCTAGACATCATTAATT | |
| | CCTAATTTTTGTTGACACTCTATCATTGATAGAGTTA | |
| | TTTTACCACTCCCTATCAGTGATAGAGAAAAGTGAA | |
| | TTATATAAAAGTGGGAGGTGCCCGAATGACGACC | |
| | CGAAATGATTGCCTAGCGTTGGATGCACAGGAC | |
| | AGTCTGGCTCCGCTGCGCCAACAATTTGCGCTG | |
| | CCGGAGGGTGTGATATACCTGGATGGCAATTCG | |
| | CTGGGCGCACGTCCGGTAGCTGCGCTGGCTCGC | |
| | GCGCAGGCTGTGATCGCAGAAGAATGGGGCAA | |
| | CGGGTTGATCCGTTCATGGAACTCTGCGGGCTG | |
| | GCGTGATCTGTCTGAACGCCTGGGTAATCGCCT | |
| | GGCTACCCTGATTGGTGCGCGCGATGGGGAAGT | |
| | AGTTGTTACTGATACCACCTCGATTAATCTGTTT | |
| | AAAGTGCTGTCAGCGGCGCTGCGCGTGCAAGCT | |
| | ACCCGTAGCCCGGAGCGCCGTGTTATCGTGACT | |
| | GAGACCTCGAATTTCCCGACCGACCTGTATATT | |
| | GCGGAAGGGTTGGCGGATATGCTGCAACAAGG | |
| | TTACACTCTGCGTTTGGTGGATTCACCGGAAGA | |
| | GCTGCCACAGGCTATAGATCAGGACACCGCGGT | |
| | GGTGATGCTGACGCACGTAAATTATAAAACCGG | |
| | TTATATGCACGACATGCAGGCTCTGACCGCGTT | |
| | GAGCCACGAGTGTGGGGCTCTGGCGATTTGGGA | |
| | TCTGGCGCACTCTGCTGGCGCTGTGCCGGTGGA | |
| | CCTGCACCAAGCGGGCGCGGACTATGCGATTGG | |
| | CTGCACGTACAAATACCTGAATGGCGGCCCGGG | |
| | TTCGCAAGCGTTTGTTTGGGTTTCGCCGCAACT | |
| | GTGCGACCTGGTACCGCAGCCGCTGTCTGGTTG | |
| | GTTCGGCCATAGTCGCCAATTCGCGATGGAGCC | |
| | GCGCTACGAACCTTCTAACGGCATTGCTCGCTA | |
| | TCTGTGCGGCACTCAGCCTATTACTAGCTTGGC | |
| | TATGGTGGAGTGCGGCCTGGATGTGTTTGCGCA | |
| | GACGGATATGGCTTCGCTGCGCCGTAAAAGTCT | |
| | GGCGCTGACTGATCTGTTCATCGAGCTGGTTGA | |
| | ACAACGCTGCGCTGCACACGAACTGACCCTGGT | |
| | TACTCCACGTGAACACGCGAAACGCGGCTCTCA | |
| | CGTGTCTTTTGAACACCCCGAGGGTTACGCTGT | |
| | TATTCAAGCTCTGATTGATCGTGGCGTGATCGG | |
| | CGATTACCGTGAGCCACGTATTATGCGTTTCGG | |
| | TTTCACTCCTCTGTATACTACTTTTACGGAAGTT | |
| | TGGGATGCAGTACAAATCCTGGGCGAAATCCTG | |
| | GATCGTAAGACTTGGGCGCAGGCTCAGTTTCAG | |
| | GTGCGCCACTCTGTTACTTAAGGAG | |

In other embodiments, human kynureninase is used (Table XXX).

Table Constructs for the Expression of Human Kynureninase

| Description | Sequence |
|---|---|
| Human | ATGGAGCCTTCATCTTTAGAACTGCCAGCGGACACGGT |
| kynureninase | GCAGCGCATCGCGGCGGAACTGAAGTGCCATCCGACT |
| codon optimized | GATGAGCGTGTGGCGCTGCATCTGGACGAAGAAGATA |
| for expression in | AACTGCGCCACTTTCGTGAATGTTTTTATATTCCTAAAA |
| E coli | TTCAAGACTTGCCGCCGGTAGATTTGAGTCTCGTTAAC |
| SEQ ID NO: | AAAGATGAAAACGCGATCTACTTTCTGGGCAACTCTCT |
| XXX | GGGTCTGCAACCAAAAATGGTTAAAACGTACCTGGAG |
| | GAAGAACTGGATAAATGGGCAAAAATCGCGGCTTATG |
| | GTCACGAAGTGGGCAAGCGTCCTTGGATTACTGGCGAC |
| | GAGTCTATTGTGGGTTTGATGAAAGATATTGTGGGCGC |
| | GAATGAAAAGGAAATTGCACTGATGAATGCTCTGACC |
| | GTTAATCTGCACCTGCTGATGCTGTCTTTTTTTAAACCG |
| | ACCCCGAAACGCTACAAAATACTGCTGGAAGCGAAAG |
| | CGTTTCCGTCGGATCACTATGCTATAGAAAGTCAACTG |
| | CAGTTGCATGGTCTGAATATCGAGGAATCTATGCGCAT |
| | GATTAAACCGCGTGAGGGTGAAGAAACGCTGCGTATT |
| | GAAGACATTCTGGAAGTTATTGAAAAAGAAGGTGATT |
| | CTATCGCAGTTATACTGTTTTCTGGCGTGCACTTTTATA |
| | CAGGTCAGCACTTCAATATCCCGGCAATCACTAAAGCG |
| | GGGCAGGCAAAAGGCTGCTATGTTGGTTTTGACCTGGC |
| | GCATGCAGTGGGGAATGTTGAACTGTATCTGCACGATT |
| | GGGGCGTTGATTTCGCGTGTTGGTGTAGCTACAAATAT |
| | CTGAACGCTGGCGCGGGTGGCATTGCTGGCGCTTTTAT |
| | TCACGAAAAACACGCGCACACCATTAAACCGGCTCTG |
| | GTTGGCTGGTTCGGTCATGAGCTGAGTACTCGCTTTAA |
| | AATGGATAACAAACTGCAATTGATTCCGGGTGTTTGCG |
| | GCTTCCGTATCAGCAATCCGCCGATTCTGCTGGTTTGC |
| | AGCCTGCACGCTAGTCTGGAAATCTTTAAGCAGGCGAC |
| | TATGAAAGCGCTGCGCAAAAAATCTGTGCTGCTGACCG |
| | GCTATCTGGAGTATCTGATCAAACACAATTATGGCAAA |
| | GATAAAGCTGCAACTAAAAAACCGGTAGTGAACATTA |
| | TCACCCCCTCACACGTGGAGGAGCGCGGTTGTCAGCTG |
| | ACTATTACTTTCAGTGTACCTAATAAAGATGTGTTCCA |
| | GGAACTGGAAAAACGCGGCGTTGTTTGTGATAAACGT |
| | AACCCGAATGGTATTCGCGTGGCTCCTGTGCCGCTGTA |
| | CAATTCATTCCACGATGTTTATAAATTCACCAACCTGC |
| | TGACTTCTATTCTCGACAGTGCTGAGACTAAAAATTAA |
| Human | TAATTCCTAATTTTTGTTGACACTCTATCATTGATAGAG |
| kynureninase, | TTATTTTACCACTCCCTATCAGTGATAGAGAAAAGTGA |
| codon optimized | ATATCAAGACACGAGGAGGTAAGATTATGGAGCCTTC |
| for expression in | ATCTTTAGAACTGCCAGCGGACACGGTGCAGCGCATCG |
| E. coli driven by | CGGCGGAACTGAAGTGCCATCCGACTGATGAGCGTGT |
| a Tet inducible | GGCGCTGCATCTGGACGAAGAAGATAAACTGCGCCAC |
| promoter | TTTCGTGAATGTTTTTATATTCCTAAAATTCAAGACTTG |
| SEQ ID NO: | CCGCCGGTAGATTTGAGTCTCGTTAACAAAGATGAAAA |
| XXX | CGCGATCTACTTTCTGGGCAACTCTCTGGGTCTGCAAC |
| | CAAAAATGGTTAAAACGTACCTGGAGGAAGAACTGGA |
| | TAAATGGGCAAAAATCGCGGCTTATGGTCACGAAGTG |
| | GGCAAGCGTCCTTGGATTACTGGCGACGAGTCTATTGT |
| | GGGTTTGATGAAAGATATTGTGGGCGCGAATGAAAAG |
| | GAAATTGCACTGATGAATGCTCTGACCGTTAATCTGCA |
| | CCTGCTGATGCTGTCTTTTTTTAAACCGACCCCGAAAC |
| | GCTACAAAATACTGCTGGAAGCGAAAGCGTTTCCGTCG |
| | GATCACTATGCTATAGAAAGTCAACTGCAGTTGCATGG |
| | TCTGAATATCGAGGAATCTATGCGCATGATTAAACCGC |
| | GTGAGGGTGAAGAAACGCTGCGTATTGAAGACATTCT |
| | GGAAGTTATTGAAAAAGAAGGTGATTCTATCGCAGTTA |
| | TACTGTTTTCTGGCGTGCACTTTTATACAGGTCAGCACT |
| | TCAATATCCCGGCAATCACTAAAGCGGGGCAGGCAAA |
| | AGGCTGCTATGTTGGTTTTGACCTGGCGCATGCAGTGG |
| | GGAATGTTGAACTGTATCTGCACGATTGGGGCGTTGAT |
| | TTCGCGTGTTGGTGTAGCTACAAATATCTGAACGCTGG |
| | CGCGGGTGGCATTGCTGGCGCTTTTATTCACGAAAAAC |
| | ACGCGCACACCATTAAACCGGCTCTGGTTGGCTGGTTC |
| | GGTCATGAGCTGAGTACTCGCTTTAAAATGGATAACAA |
| | ACTGCAATTGATTCCGGGTGTTTGCGGCTTCCGTATCA |
| | GCAATCCGCCGATTCTGCTGGTTTGCAGCCTGCACGCT |
| | AGTCTGGAAATCTTTAAGCAGGCGACTATGAAAGCGCT |
| | GCGCAAAAAATCTGTGCTGCTGACCGGCTATCTGGAGT |
| | ATCTGATCAAACACAATTATGGCAAAGATAAAGCTGC |
| | AACTAAAAAACCGGTAGTGAACATTATCACCCCCTCAC |
| | ACGTGGAGGAGCGCGGTTGTCAGCTGACTATTACTTTC |
| | AGTGTACCTAATAAAGATGTGTTCCAGGAACTGGAAA |
| | AACGCGGCGTTGTTTGTGATAAACGTAACCCGAATGGT |
| | ATTCGCGTGGCTCCTGTGCCGCTGTACAATTCATTCCA |
| | CGATGTTTATAAATTCACCAACCTGCTGACTTCTATTCT |
| | CGACAGTGCTGAGACTAAAAATTAA |
| Human | TAAGACCCACTTTCACATTTAAGTTGTTTTTCTAATCCG |
| kynureninase | CATATGATCAATTCAAGGCCGAATAAGAAGGCTGGCT |
| codon optimized | CTGCACCTTGGTGATCAAATAATTCGATAGCTTGTCGT |
| for expression in | AATAATGGCGGCATACTATCAGTAGTAGGTGTTTCCCT |
| E. coli (no | TTCTTCTTTAGCGACTTGATGCTCTTGATCTTCCAATAC |
| underline, no | GCAACCTAAAGTAAAATGCCCCACAGCGCTGAGTGCA |
| italic) driven by | TATAATGCATTCTCTAGTGAAAAACCTTGTTGGCATAA |
| a Tet inducible | AAAGGCTAATTGATTTTCGAGAGTTTCATACTGTTTTTC |
| promoter | TGTAGGCCGTGTACCTAAATGTACTTTTGCTCCATCGC |
| (underlined | GATGACTTAGTAAAGCACATCTAAAACTTTTAGCGTTA |
| italic) with RBS | TTACGTAAAAAATCTTGCCAGCTTTCCCCTTCTAAAGG |
| (italic, and tetR | GCAAAAGTGAGTATGGTGCCTATCTAACATCTCAATGG |
| in reverse | CTAAGGCGTCGAGCAAAGCCCGCTTATTTTTTACATGC |
| orientation | CAATACAATGTAGGCTGCTCTACACCTAGCTTCTGGGC |
| (underlined) | GAGTTTACGGGTTGTTAAACCTTCGATTCCGACCTCAT |
| SEQ ID NO: | TAAGCAGCTCTAATGCGCTGTTAATCACTTTACTTTTAT |
| XXX | CTAATCTAGACATCATTAATTCCTAATTTTTGTTGACACTC |
| | TATCATTGATAGAGTTATTTTACCACTCCCTATCAGTGATAG |
| | AGAAAAGTGAATATCAAGACACGAGGAGGTAAGATTATGG |
| | AGCCTTCATCTTTAGAACTGCCAGCGGACACGGTGCAG |
| | CGCATCGCGGCGGAACTGAAGTGCCATCCGACTGATG |
| | AGCGTGTGGCGCTGCATCTGGACGAAGAAGATAAACT |
| | GCGCCACTTTCGTGAATGTTTTTATATTCCTAAAATTCA |
| | AGACTTGCCGCCGGTAGATTTGAGTCTCGTTAACAAAG |
| | ATGAAAACGCGATCTACTTTCTGGGCAACTCTCTGGGT |
| | CTGCAACCAAAAATGGTTAAAACGTACCTGGAGGAAG |
| | AACTGGATAAATGGGCAAAAATCGCGGCTTATGGTCA |
| | CGAAGTGGGCAAGCGTCCTTGGATTACTGGCGACGAG |
| | TCTATTGTGGGTTTGATGAAAGATATTGTGGGCGCGAA |
| | TGAAAAGGAAATTGCACTGATGAATGCTCTGACCGTTA |
| | ATCTGCACCTGCTGATGCTGTCTTTTTTTAAACCGACCC |
| | CGAAACGCTACAAAATACTGCTGGAAGCGAAAGCGTT |
| | TCCGTCGGATCACTATGCTATAGAAAGTCAACTGCAGT |
| | TGCATGGTCTGAATATCGAGGAATCTATGCGCATGATT |
| | AAACCGCGTGAGGGTGAAGAAACGCTGCGTATTGAAG |
| | ACATTCTGGAAGTTATTGAAAAAGAAGGTGATTCTATC |
| | GCAGTTATACTGTTTTCTGGCGTGCACTTTTATACAGGT |
| | CAGCACTTCAATATCCCGGCAATCACTAAAGCGGGGC |
| | AGGCAAAAGGCTGCTATGTTGGTTTTGACCTGGCGCAT |
| | GCAGTGGGGAATGTTGAACTGTATCTGCACGATTGGGG |
| | CGTTGATTTCGCGTGTTGGTGTAGCTACAAATATCTGA |
| | ACGCTGGCGCGGGTGGCATTGCTGGCGCTTTTATTCAC |
| | GAAAAACACGCGCACACCATTAAACCGGCTCTGGTTG |
| | GCTGGTTCGGTCATGAGCTGAGTACTCGCTTTAAAATG |
| | GATAACAAACTGCAATTGATTCCGGGTGTTTGCGGCTT |
| | CCGTATCAGCAATCCGCCGATTCTGCTGGTTTGCAGCC |
| | TGCACGCTAGTCTGGAAATCTTTAAGCAGGCGACTATG |
| | AAAGCGCTGCGCAAAAAATCTGTGCTGCTGACCGGCT |
| | ATCTGGAGTATCTGATCAAACACAATTATGGCAAAGAT |
| | AAAGCTGCAACTAAAAAACCGGTAGTGAACATTATCA |
| | CCCCCTCACACGTGGAGGAGCGCGGTTGTCAGCTGACT |
| | ATTACTTTCAGTGTACCTAATAAAGATGTGTTCCAGGA |
| | ACTGGAAAAACGCGGCGTTGTTTGTGATAAACGTAACC |
| | CGAATGGTATTCGCGTGGCTCCTGTGCCGCTGTACAAT |
| | TCATTCCACGATGTTTATAAATTCACCAACCTGCTGAC |
| | TTCTATTCTCGACAGTGCTGAGACTAAAAATTAA |

E. coli naturally utilizes anthranilate in its TRP biosynthetic pathway. Briefly, the TrpE (in complex with TrpD) enzyme converts chorismate into anthranilate. TrpD, TrpC, TrpA and TrpB then catalyze a five-step reaction ending with the condensation of an indole with serine to form tryptophan. Next, the kynureninase is introduced into a strain which harbors ΔtrpE (trypophan auxotrophy) deletion. By deleting the TrpE enzyme via lambda-RED recombineering, the subsequent strain of Nissle (ΔtrpE::Cm) is an auxotroph unable to grow in minimal media without supplementation of TRP or anthranilate. By expressing kynureninase in ΔtrpE::Cm (KYNase-trpE), this auxotrophy should alternatively rescued by providing KYN.

Indeed, as a proof of concept, we showed that-while Nissle does not typically utilize KYN—by introducing the kynureninase (KYNase) from Pseudomonas fluorescens (kynU) on a medium-copy plasmid under the control of the tetracycline promoter (Ptet) a new strain with this plasmid (Ptet-KYNase) was able to convert L-kynurenine into anthranilate in the presence of a Tet inducer.

TABLE 15

| | Rich | | Min + | Min + KYNU + |
|---|---|---|---|---|
| STRAIN | Media | Min Media | Anthranilate | aTc |
| Wild type | + | + | + | + |
| Nissle | | | | |
| trpE | + | − | + | − |
| trpE | + | − | + | + |
| pseudoKYNase | | | | |
| trpE hKYNase | + | − | + | − |

In a preliminary assay (Table 15), wildtype Nissle (SYN094), Nissle with a deletion of trpE, and trpE mutants expressing either the human kynureninase (hKYNase) or the Pseudomonas fluorescens kynureninase (pseudoKYNase) from a Ptet promoter on a medium-copy plasmid were grown in either rich media, minimal media (min media), minimal media with 5 mM anthranilate (Min+anthranilate) or minimal media with 10 mM kynurenine and 100 ng/uL aTc (Min+KYNU+aTc). These were grown in 1 mL of media in a deep well plate with shaking at 37° C. A positive for growth (+) in Table 15 indicates a change in optical density of >5-fold from inoculation.

The results show that in a mutant trpE (which is typically used in the tryptophan biosynthetic pathway to convert chorismate into anthranilate) background, Nissle is unable to grow in minimal media without supplementation with anthranilate (or tryptophan). When minimal media was supplemented with KYNU, the trpE mutant was also unable to grow. However, when the Pseudomonas KYNase was expressed in the trpE tryptophan-auxotroph the cells were able to grow in Min+KYNU. This indicates that Nissle is able to import L-kynurenine from the media and convert it into anthranilate using the pseudoKYNase. The human KYNase homolog was unable to support growth on M9+KYNU, most likely due to differences in substrate specificity as it has been documented that the human kynureninase prefers 3-hydroxykynurenine as a substrate (Phillips, Structure and mechanism of kynureninase. Arch Biochem Biophys. 2014 Feb. 15; 544:69-74).

Together these experiments establish that expression of the Pseudomonas fluorescens kynureninase is sufficient to rescue a trpE auxotrophy in the presence of kynurenine, as the strain is able to consume KYN into anthranilate, and upstream metabolite in the TRP biosynthetic pathway. In addition, the KYNase is also capable of providing increased resistance to the toxic tryptophan, 5-fluoro-L-tryptophan. Using the information attained here it is possible to proceed to an adapative laboratory evolution experiment to select for mutants with highly efficient and selective conversion of kynurenine to tryptophan.

### Example 61. Generation of E. Coli Mutants with Enhanced Ability to Consume L-Kynurenine and Produce Tryptophan from Kynurenine

Adaptive Laboratory Evolution was used to produce mutant bacterial strains with improved kynurenine consumption and reduced tryptophan uptake.

Prior to evolving the strains, a lower limit of kynurenine (KYN) concentration was established for use in the ALE experiment.

While lowering the KYN concentration can select for mutants capable of increasing KYN utilization, the bacterial cells still prefer to utilize free, exogenous TRP. In the tumor environment, dual-therapeutic functions can be provided by depletion of KYN and increasing local concentrations of TRP. Therefore, to evolve a strain which prefers KYN over TRP, a toxic analogue of TRP—5-fluoro-L-tryptophan (ToxTRP)—can be incorporated into the ALE experiment.

A checkerboard growth assay was performed in 96-well plates using streptomycin resistant Nissle, deltatrpE and deltatrpE pseudoKYNase with and without induction of pseudoKYNase expression using 100 ng/uL aTc. Detailed procedures used for the checkerboard assay are described in Example 14. Strains were inoculated at very dilute concentrations into M9 minimal media with varying concentrations of KYN across columns (2-fold dilutions starting at 2000 ug/mL) and varying concentrations of ToxTrp across rows (2-fold dilutions starting at 200 ug/mL). On a separate plate, the strains were grown in M9+KYN (at the same concentrations) in the absence of ToxTrp.

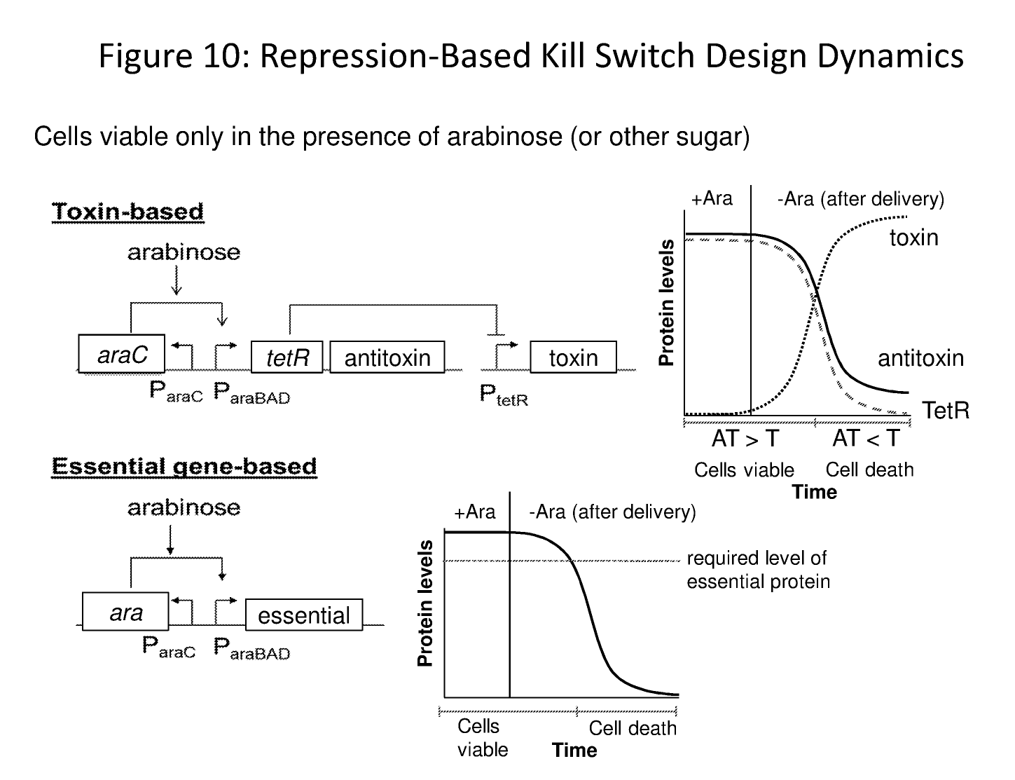

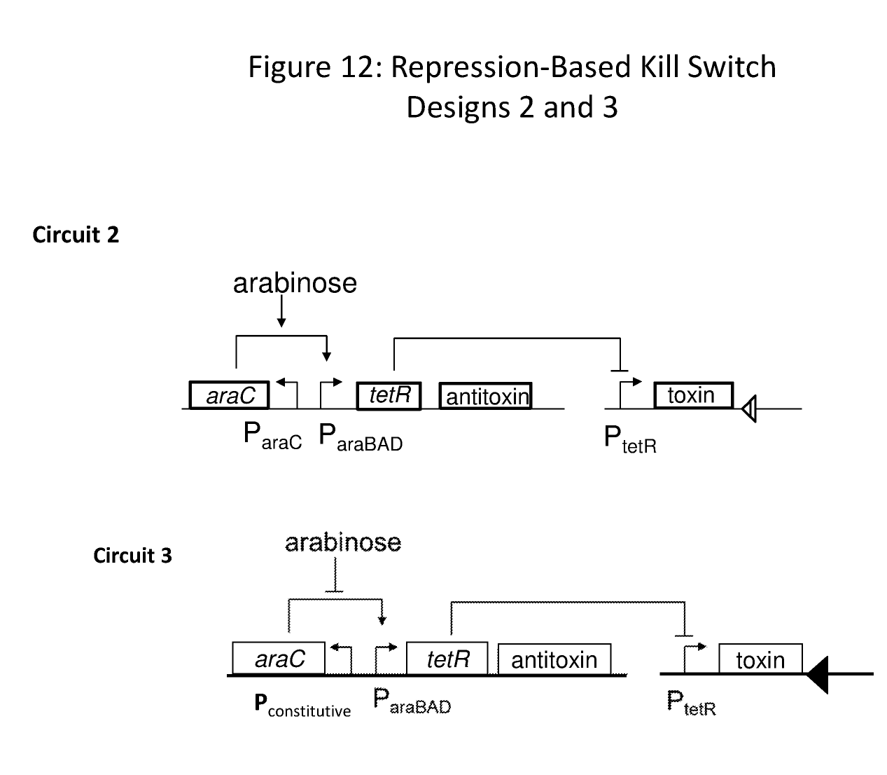

The results of the initial checkerboard assay are shown in FIG. 10, FIG. 11, and FIG. 12 as a function of optical density at 600 nm (normalized to a media blank). In FIG. 10 and FIG. 11, the X-axis shows decreasing KYNU concentration from left-to-right, while the Z-axis shows decreasing ToxTrp concentration from front-to-back with the very back row representing media with no ToxTrp. In FIG. 10, the controls and trpE strains are shown in M9+KYNU without any ToxTrp, as there was no growth detected from either strain at any concentration of ToxTrp. The results of the assay show that expression of the pseudoKYNase provides protection against toxicity of ToxTrp. More importantly, growth is permitted between 250-62.5 ug/mL of KYNU and 6.3-1.55 ug/mL of ToxTrp.

### Example 62. Checkerboard Assay and ALE Parameters

To establish the minimum concentration of L-kynurenine and maximum concentration of 5-fluoro-L-tryptophan (ToxTrp) capable of sustaining growth of the KYNase strain, using a checkerboard assay, the following protocol was used. Using a 96-well plate with M9 minimal media with glucose, KYN was supplemented decreasing across columns in 2-fold dilutions from 2000 ug/mL down to ˜1 ug/mL. In the rows, ToxTrp concentration decreased by 2-fold from 200 ug/mL down to ˜1.5 ug/mL. In one plate, Anhydrous Tetracycline (aTc) was added to a final concentration of 100 ng/uL to induce production of the KYNase. From an overnight culture, cells were diluted to an OD600=0.5 in 12 mL of TB (plus appropriate antibiotics and inducers, where applicable) and grown for 4 hours. 100 uL of cells were spun down and resuspended to an OD600=1.0. These were diluted 2000-fold and 25 uL was added to each well to bring the final volumes in each well to 100 uL. Cells were grown for roughly 20 hours with static incubation at 37 C then growth was assessed by OD600, making sure readings fell within linear range (0.05-1.0).

### Example 63. Determination of ALE Parameters

Once identified, the highest concentrations of ToxTrp and lowest concentration of kynurenine capable of supporting growth becomes the starting point for ALE. The ALE parental strain was chosen by culturing the KYNase strain on M9 minimal media supplemented with glucose and L-kynurenine (referred to as M9+KYN from here on). A single colony was selected, resuspended in 20 uL of sterile phosphate-buffered saline solution. This colony was then used to inoculate three cultures of M9+KYN, grown into late-logarithmic phase and optical density determined at 600 nm. These cultures were then diluted to 10³ in 4 rows of a 96-well deep-well plate with 1 mL of M9+KYN. Each one of the four rows has a different ToxTrp (increasing 2-fold), while each column has decreasing concentrations of KYN (by 2-fold). Each morning and evening this plate is diluted back to 10³ using the well in which the culture has grown to just below saturation so that the culture is always in logarithmic growth. This process is repeated until a change in growth rate is no longer detected. Once no growth rate increases are detected (usually around 10¹¹ Cumulative Cell Divisions) the culture is plated onto M9+KYN (Lee, et al., Cumulative Number of Cell Divisions as a Meaningful Timescale for Adaptive Laboratory Evolution of Escherichia coli. PLoS ONE 6, e26172; 2011). Individual colonies are selected and screened in M9+KYN+ToxTrp media to confirm increased growth rate phenotype. Once mutants with significantly increased growth rate on M9+KYN are isolated, genomic DNA can be isolated and sent for whole genome sequencing to reveal the mutations responsible for phenotype. All culturing is done shaking at 350 RPM at 37° C.

The resulting best performing strain can them be whole genome sequenced in order to deconvolute the contributing mutations. In some embodiments, Lambda-RED can be performed in order to reintroduce TrpE, to inactivate Trp regulation (trpR, tyrR, transcriptional attenuators) to up-regulate TrpABCDE expression and increase chorismate production. The resulting strain prefers external KYN over to external TRP, efficiently converts KYN into TRP, and also now overproduces TRP.

### Example 64. ALE

First, strains were generated, which comprise the trpE knock out and integrated constructs for the expression of Pseudomonas fluorescens KYNase driven by a constitutive promoter (Table XXX). KYNase constructs were integrated at the HA3/4 site, and two different promoters were used; the promoter of the endogenous lpp gene was used in parental strain SYN2027 (HA3/4::Plpp-pKYNase KanR TrpE::CmR) and the synthetic pSynJ23119 was used in parental strain SYN2028 (HA3/4::PSynJ23119-pKYNase KanR TrpE::CmR). These strains were generated so that a strain would be evolved, which would comprise a chromosomally integrated version of Pseudomonas fluorescens KYNase.

TABLE XXX
Constructs for Constitutive Expression of
Pseudomonas fluorescens Kynureninase

| Description | Sequence |
|---|---|
| SYN23119 promoter | GGAAAATTTTTTTAAAAAAAAAACTTGACAGCT |
| | AGCTCAGTCCTTGGTATAATGCTAGCACGAA |
| SEQ ID NO: XXX | |
| RBS | TTATATAAAAGTGGGAGGTGCCCGA |
| SEQ ID NO: XXX | |
| SYN23119 promoter with | GGAAAATTTTTTTAAAAAAAAAACTTGACAGCT |
| RBS | AGCTCAGTCCTTGGTATAATGCTAGCACGAAGT |
| SEQ ID NO: XXX | GAATTATATAAAAGTGGGAGGTGCCCGA |
| Pseudomonas | GGAAAATTTTTTTAAAAAAAAAACTTGACAGCT |
| fluorescens, codon optimized | AGCTCAGTCCTTGGTATAATGCTAGCACGAAGT |
| for expression in E. coli, | GAATTATATAAAAGTGGGAGGTGCCCGAATGA |
| driven by the SYN23119 | CGACCCGAAATGATTGCCTAGCGTTGGATGCAC |
| | AGGACAGTCTGGCTCCGCTGCGCCAACAATTTG |
| | CGCTGCCGGAGGGTGTGATATACCTGGATGGCA |
| | ATTCGCTGGGCGCACGTCCGGTAGCTGCGCTGG |
| Construct can be | CTCGCGCGCAGGCTGTGATCGCAGAAGAATGG |
| expressed from a | GGCAACGGGTTGATCCGTTCATGGAACTCTGCG |
| plasmid, e.g., p15 or | GGCTGGCGTGATCTGTCTGAACGCCTGGGTAAT |
| can be integrated into | CGCCTGGCTACCCTGATTGGTGCGCGCGATGGG |
| the chromosome, | GAAGTAGTTGTTACTGATACCACCTCGATTAAT |
| e.g., at the HA3/4 site | CTGTTTAAAGTGCTGTCAGCGGCGCTGCGCGTG |
| SEQ ID NO: XXX | CAAGCTACCCGTAGCCCGGAGCGCCGTGTTATC |
| | GTGACTGAGACCTCGAATTTCCCGACCGACCTG |
| | TATATTGCGGAAGGGTTGGCGGATATGCTGCAA |
| | CAAGGTTACACTCTGCGTTTGGTGGATTCACCG |
| | GAAGAGCTGCCACAGGCTATAGATCAGGACAC |
| | CGCGGTGGTGATGCTGACGCACGTAAATTATAA |
| | AACCGGTTATATGCACGACATGCAGGCTCTGAC |
| | CGCGTTGAGCCACGAGTGTGGGGCTCTGGCGAT |
| | TTGGGATCTGGCGCACTCTGCTGGCGCTGTGCC |
| | GGTGGACCTGCACCAAGCGGGCGCGGACTATG |
| | CGATTGGCTGCACGTACAAATACCTGAATGGCG |
| | GCCCGGGTTCGCAAGCGTTTGTTTGGGTTTCGC |
| | CGCAACTGTGCGACCTGGTACCGCAGCCGCTGT |
| | CTGGTTGGTTCGGCCATAGTCGCCAATTCGCGA |
| | TGGAGCCGCGCTACGAACCTTCTAACGGCATTG |
| | CTCGCTATCTGTGCGGCACTCAGCCTATTACTA |
| | GCTTGGCTATGGTGGAGTGCGGCCTGGATGTGT |
| | TTGCGCAGACGGATATGGCTTCGCTGCGCCGTA |
| | AAAGTCTGGCGCTGACTGATCTGTTCATCGAGC |
| | TGGTTGAACAACGCTGCGCTGCACACGAACTGA |
| | CCCTGGTTACTCCACGTGAACACGCGAAACGCG |
| | GCTCTCACGTGTCTTTTGAACACCCCGAGGGTT |
| | ACGCTGTTATTCAAGCTCTGATTGATCGTGGCG |
| | TGATCGGCGATTACCGTGAGCCACGTATTATGC |
| | GTTTCGGT |
| Lpp promoter from | ATAAGTGCCTTCCCATCAAAAAAATATTCTCAA |
| E. coli | CATAAAAAACTTTGTGTAATACTTGTAACGCTA |
| SEQ ID NO: XXX | |
| RBS | TTATATAAAAGTGGGAGGTGCCCGA |
| SEQ ID NO: XXX | |
| Lpp promoter from | ATAAGTGCCTTCCCATCAAAAAAATATTCTCAA |
| E. coli | CATAAAAAACTTTGTGTAATACTTGTAACGCTA |
| | GTGAATTATATAAAAGTGGGAGGTGCCCGA |
| SEQ ID NO: XXX | |
| Pseudomonas | ATAAGTGCCTTCCCATCAAAAAAATATTCTCAA |
| fluorescens | CATAAAAAACTTTGTGTAATACTTGTAACGCTA |
| kynureninase driven | GTGAATTATATAAAAGTGGGAGGTGCCCGAAT |
| by | GACGACCCGAAATGATTGCCTAGCGTTGGATGC |
| Lpp promoter from | ACAGGACAGTCTGGCTCCGCTGCGCCAACAATT |
| E. coli | TGCGCTGCCGGAGGGTGTGATATACCTGGATGG |
| | CAATTCGCTGGGCGCACGTCCGGTAGCTGCGCT |
| Construct can be | GGCTCGCGCGCAGGCTGTGATCGCAGAAGAAT |
| expressed from a | GGGGCAACGGGTTGATCCGTTCATGGAACTCTG |
| plasmid, e.g., p15 or | CGGGCTGGCGTGATCTGTCTGAACGCCTGGGTA |
| can be integrated into | ATCGCCTGGCTACCCTGATTGGTGCGCGCGATG |
| the chromosome, | GGGAAGTAGTTGTTACTGATACCACCTCGATTA |
| e.g., at the HA3/4 site | ATCTGTTTAAAGTGCTGTCAGCGGCGCTGCGCG |
| SEQ ID NO: XXX | TGCAAGCTACCCGTAGCCCGGAGCGCCGTGTTA |
| | TCGTGACTGAGACCTCGAATTTCCCGACCGACC |
| | TGTATATTGCGGAAGGGTTGGCGGATATGCTGC |
| | AACAAGGTTACACTCTGCGTTTGGTGGATTCAC |
| | CGGAAGAGCTGCCACAGGCTATAGATCAGGAC |
| | ACCGCGGTGGTGATGCTGACGCACGTAAATTAT |
| | AAAACCGGTTATATGCACGACATGCAGGCTCTG |
| | ACCGCGTTGAGCCACGAGTGTGGGGCTCTGGCG |
| | ATTTGGGATCTGGCGCACTCTGCTGGCGCTGTG |
| | CCGGTGGACCTGCACCAAGCGGGCGCGGACTA |
| | TGCGATTGGCTGCACGTACAAATACCTGAATGG |
| | CGGCCCGGGTTCGCAAGCGTTTGTTTGGGTTTC |
| | GCCGCAACTGTGCGACCTGGTACCGCAGCCGCT |
| | GTCTGGTTGGTTCGGCCATAGTCGCCAATTCGC |
| | GATGGAGCCGCGCTACGAACCTTCTAACGGCAT |
| | TGCTCGCTATCTGTGCGGCACTCAGCCTATTACT |
| | AGCTTGGCTATGGTGGAGTGCGGCCTGGATGTG |
| | TTTGCGCAGACGGATATGGCTTCGCTGCGCCGT |
| | AAAAGTCTGGCGCTGACTGATCTGTTCATCGAG |
| | CTGGTTGAACAACGCTGCGCTGCACACGAACTG |
| | ACCCTGGTTACTCCACGTGAACACGCGAAACGC |
| | GGCTCTCACGTGTCTTTTGAACACCCCGAGGGT |
| | TACGCTGTTATTCAAGCTCTGATTGATCGTGGC |
| | GTGATCGGCGATTACCGTGAGCCACGTATTATG |
| | CGTTTCGGTTTCACTCCTCTGTATACTACTTTTA |
| | CGGAAGTTTGGGATGCAGTACAAATCCTGGGCG |
| | AAATCCTGGATCGTAAGACTTGGGCGCAGGCTC |
| | AGTTTCAGGTGCGCCACTCTGTTACTTAA |

These strains were validated in the checkerboard assay described in EXAMPLE 15 to have similar ALE parameters to their plasmid-based Ptet counterpart. Lower limit of kynurenine (KYN) and ToxTrp concentration for use in the ALE experiment were established using the checkerboard assay described above herein, and lower limit concentrations corresponded to those observed for the strains expressing tet inducible KYNase from a medium copy plasmid.

Mutants derived from parental strains SYN2027 and SYN2028 were evolved by passaging in lowering concentrations of KYN and three different ToxTrp concentrations as follows.

The ALE parental strains were cultured on plates with M9 minimal media supplemented with glucose and L-kynurenine (M9+KYN). A single colony from each parent was selected, resuspended in 20 uL of sterile phosphate-buffered saline solution. This colony was then used to inoculate two cultures of M9+KYN, grown into late-logarithmic phase and the optical density was determined at 600 nm. These cultures were then diluted to 10³ in 3 columns of a 96-well deep-well plate with 1 mL of M9+KYNU. Each one of the three rows had different ToxTrp concentrations (increasing 2-fold), while each column had decreasing concentrations of KYN (by 2-fold). Every 12 hours, the plate was diluted back using 30 uL from the well in which the culture had grown to an OD600 of roughly 0.1. This process was repeated for five days, and then the ToxTrp concentrations were doubled to maintain selection pressure. After two weeks' time, no growth rate increases were detected and the culture was plated onto M9+KYN. All culturing was done shaking at 350 RPM at 37° C. Individual colonies were selected and screened in M9+KYN+ToxTrp media to confirm increased growth rate phenotype.

Two replicates for each parental strain (SYN20207-R1, SYN2027-R2, SYN2028-R1, and SYN2028-R2) were selected and assayed for kynurenine production.

Briefly, overnight cultures were diluted 1:100 in 400 ml LB and let grow for 4 hours. Next, 2 ml of the culture was spun down and resuspended in 2 ml M9 buffer. The OD600 of the culture was measured ( 1/100 dilution in PBS). The necessary amount of cell culture for a 3 ml assay targeting starting cell count of ˜OD 0.8 (˜1E8) was spun down. The cell pellet was resuspended in M9+0.5% glucose+75 uM KYN in the assay volume (3 ml) in a culture tube. 220 ul was removed in triplicate at each time point (t=0, 2, and 3 hours) into conical shaped 96WP, and 4 ul were removed for cfu measurement at each time point. At each time point, the sample was spun down in the conical 96WP for 5 minutes at 3000 g, and 200 ul were transferred from each well into a clear, flat-bottomed, 96WP. A kynurenine standard curve and blank sample was prepared in the same plate. Next, 40 ul of 30% Tri-Chloric Acid (v/v) was added to each well and mixed by pipetting up and down. The plat was sealed with aluminum foil and incubated at 60 C for 15 minutes. The plate was the spun down at 11500 rpm, at 4 C, for 15 minutes, and 125 ul from each well were aliquoted and mixed with 125 ul of 2% Ehrlich's reagent in glacial acetic acid in another 96WP. Samples were mixed pipetting up and down and the absorbance was measured at OD480. Growth rates are shown for parental strains SYN2027 and SYN2028 and the corresponding evolved strains in FIG. 13.

### Example 65. Kynurenine Consuming Strains Decrease Tumoral Kynurenine Levels in the CT26 Murine Tumor Model

The ability of genetically engineered bacteria comprising kynureninase from Pseudomonas fluorescens to consume kynurenine in vivo in the tumor environment was assessed. SYN1704, an E. coli Nissle strain comprising a deletion in Trp:E and a medium copy plasmid expressing kynureninase from Pseudomonas fluorescens under control of a constitutive promoter (Nissle delta TrpE::CmR+Pconstitutive-Pseudomonas KYNU KanR) was used for in a first study (Study 1).

In a second study (Study 2) the activity of SYN2028, an E. coli Nissle strain comprising a deletion in Trp:E and an integrated construct expressing kynureninase from Pseudomonas fluorescens under the control of a constitutive promoter (Nissle HA3/4::PSynJ23119-pKYNase KanR TrpE::CmR) was assessed.

In both studies, CT26 cells obtained from ATCC were cultured according to guidlelines provided. Approximately—1e6 cells/mouse in PBS were implanted subcutaneously into the right flank of each animal (BalbC/J (female, 8 weeks)), and tumor growth was monitored for approximately 10 days. When the tumors reached about ˜100-150 mm3, animals were randomized into groups for dosing.

For intratumoral injection, bacteria were grown in LB broth until reaching an absorbance at 600 nm (A600 nm) of 0.4 (corresponding to 2×108 colony-forming units (CFU)/mL) and washed twice in PBS. The suspension was diluted in PBS or saline so that 100 microL can be injected at the appropriate doses intratumorally into tumor-bearing mice.

(a) Study 1:

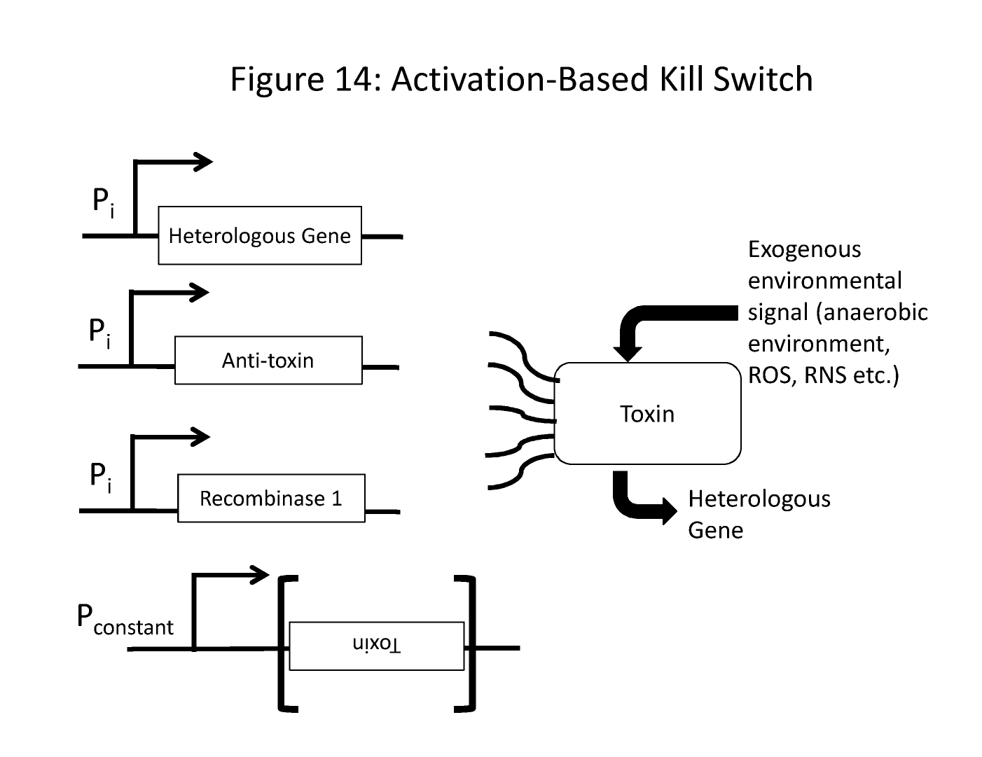

Approximately 10 days after CT 26 implanation, bacteria were suspended in 0.1 ml of PBS and mice were injected (5e6 cells/mouse) with 100 ul intratumorally as follows: Group 1-Vehicle Control (n=8), Group 2-SYN94 (n=8), and Group 3-SYN1704 (n=8). From Day 2 until study end, animals were dosed intratumorally biweekly with 100 ul of vehicle control or bacteria at 5e6 cells/mouse. Animals were weighed and the tumor volume measured twice weekly. Animals were euthanized when the tumors reached ˜2000 mm3 and kynurenine concentrations were measured by LC/MS as described herein. Results are shown in FIG. 14A. A significant reduction in intra tumor concentration was observed for the kynurenine consuming strain SYN1704 and for wild type E. coli Nissle. Intratumoral kynurenine levels were reduced in SYN1704, as compared to wild type Nissle, although the difference did not reach significance due to one outlier.

(b) Study 2:

Approximately 10 days after CT 26 implanation, bacteria were suspended in 0.1 ml of saline and mice were injected (1e8 cells/mouse) with the bacterial suspension intratumorally as follows: Group 1-Vehicle Control (n=10), Group 2-SYN94 (n=10), Group 3-SYN2028 (n=10). Group 5 (n=10) received INCB024360 (IDO inhibitor) via oral gavage as a control twice daily. From Day 2 until study end, animals were dosed intratumorally biweekly with 100 ul of vehicle control or bacteria at 1e8 cells/mouse. Animals were weighed and the tumor volume measured twice weekly. Group 5 received INCB024360 via oral gavage as a control twice daily until study end. Animals were euthanized when the tumors reached—2000 mm3 Tumor fragments were placed in pre-weighed bead-buster tubes and store don ice for analysis. Kynurenine concentrations were measured by LC/MS as described herein. Results are shown in FIG. 14B. A significant reduction in intra tumor concentration was observed for the kynurenine consuming strain SYN2028 as compared to wild type Nissle or wild type control. Intratumoral kynurenine levels seen in SYN2028 were similar to those observed for the IDO inhibitor INCB024360.

### Example 66. Generation of Indole Propionic Acid Strain and In Vitro Indole Production

To facilitate inducible production of indole propionic acid (IPA) in Escherichia coli Nissle, 6 genes allowing the production of indole propionic acid from tryptophan, as well as transcriptional and translational elements, are synthesized (Gen9, Cambridge, Mass.) and cloned into vector pBR322 under a tet inducible promoter. In other embodiments, the IPA synthesis cassette is put under the control of an FNR, RNS or ROS promoter, e.g., described herein, or other promoter induced by conditions in the healthy or diseased gut, e.g., inflammatory conditions. For efficient translation of IPA synthesis genes, each synthetic gene in the cassette is separated by a 15 base pair ribosome binding site derived from the T7 promoter/translational start site.

The IPA synthesis cassette comprises TrpDH (tryptophan dehydrogenase from Nostoc punctiforme NIES-2108), FldH1/FldH2 (indole-3-lactate dehydrogenase from Clostridium sporogenes), FldA (indole-3-propionyl-CoA:indole-3-lactate CoA transferase from Clostridium sporogenes), FldBC (indole-3-lactate dehydratase from Clostridium sporogenes), FldD (indole-3-acrylyl-CoA reductase from Clostridium sporogenes), and AcuI (acrylyl-CoA reductase from Rhodobacter sphaeroides).

The tet inducible IPA construct described above is transformed into E. coli Nissle as described herein and production of IPA is assessed. In certain embodiments, E. coli Nissle strains containing the IPA synthesis cassette described further comprise a tryptophan synthesis cassette. In certain embodiments, the strains comprise a feedback resistant version of AroG and TrpE to achieve greater Trp production. In certain embodiments, additionally, the tnaA gene (tryptophanase converting Trp into indole) is deleted.

All incubations are performed at 37° C. LB-grown overnight cultures of E. coli Nissle transformed with the IPA biosynthesis construct alone or in combination with a tryptophan biosynthis construct and feedback resistant AroG and TrpE are subcultured 1:100 into 10 mL of M9 minimal medium containing 0.5% glucose and grown shaking (200 rpm) for 2 h, at which time anhydrous tetracycline (ATC) is added to cultures at a concentration of 100 ng/mL to induce expression of the the IPA biosynthesis and tryptophan biosynthesis constructs. After 2 hours of induction, cells are spun down, supernatant is discarded, and the cells are resuspended in M9 minimal media containing 0.5% glucose. Culture supernatant is then analyzed at predetermined time points (e.g., 0 up to 24 hours) by LC-MS to assess levels of IPA.

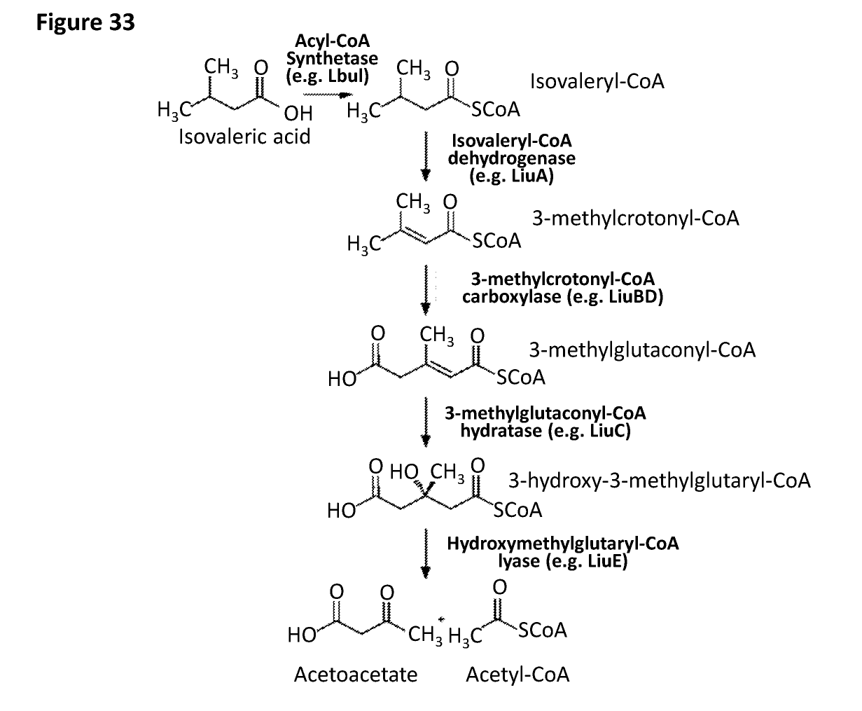

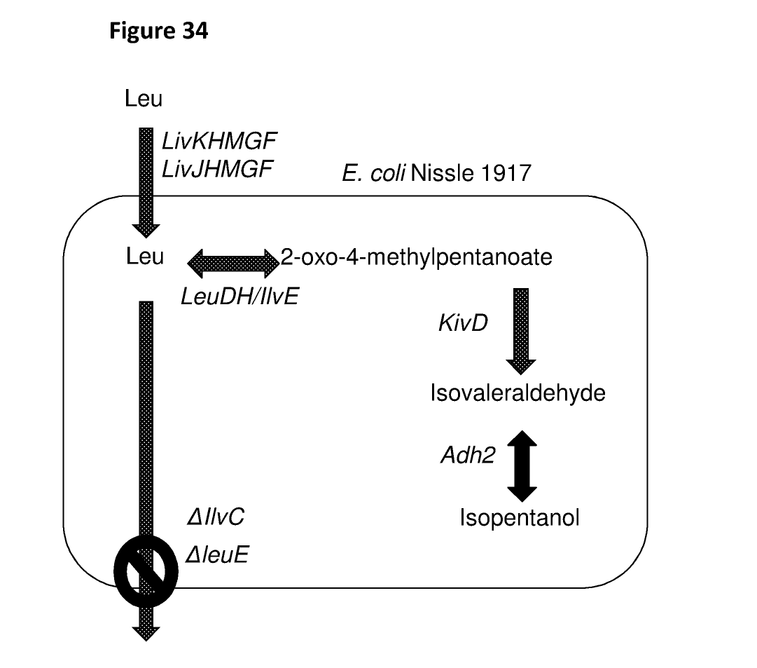

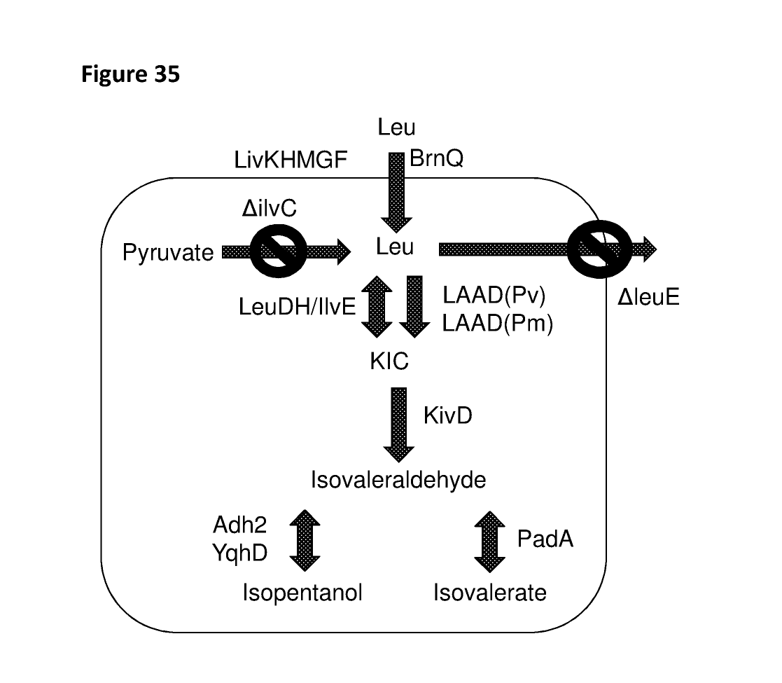

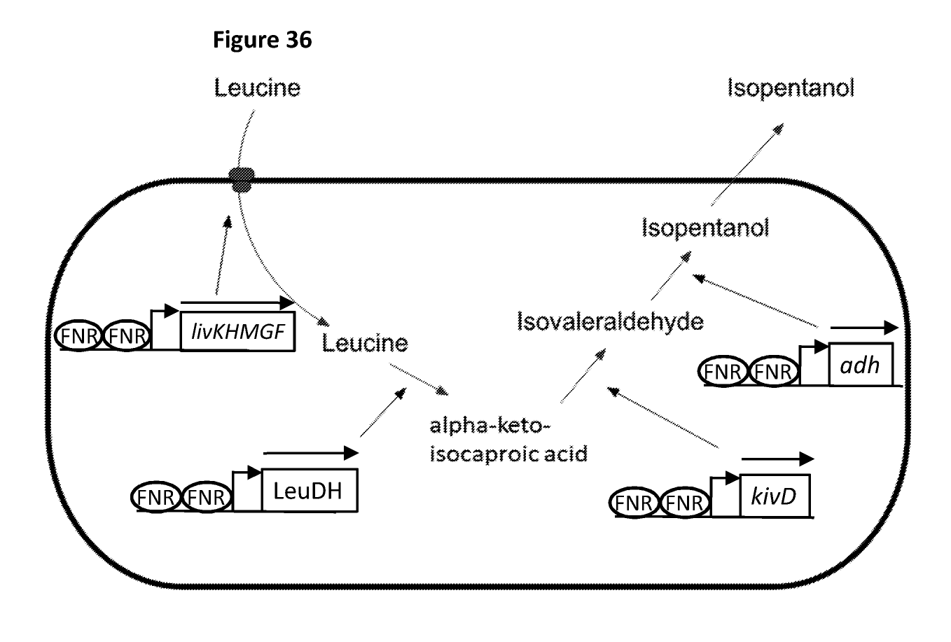

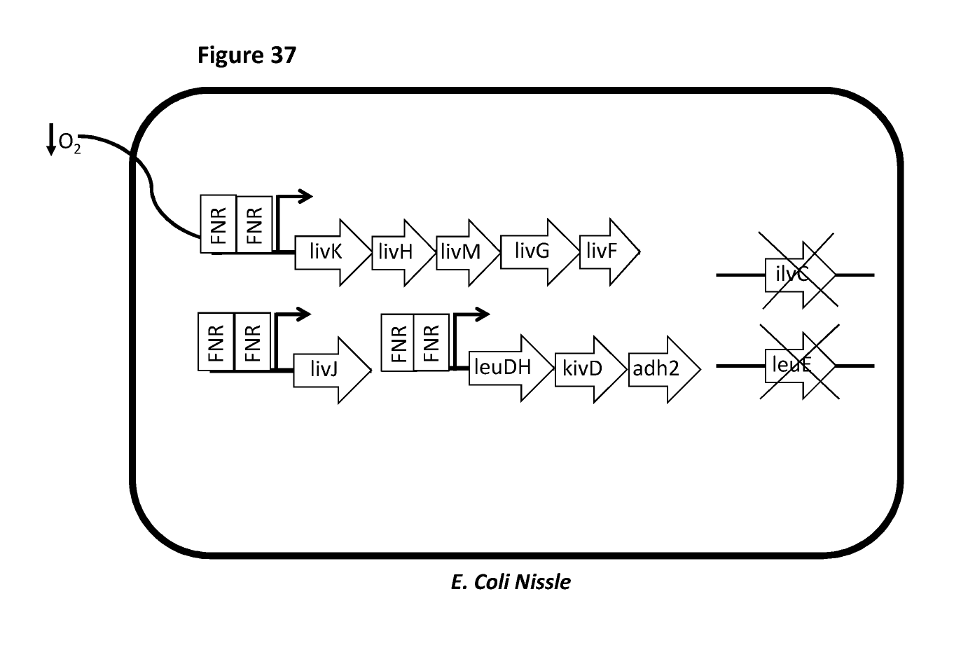

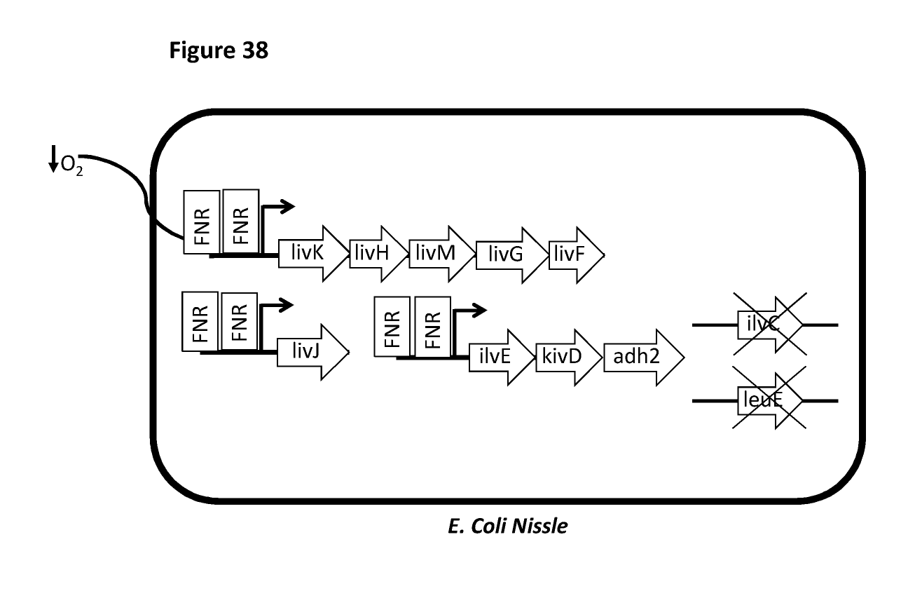

Production of IPA is also assessed in E. coli Nissle strains containing the IPA and tryptophan cassettes both driven by an RNS promoter e.g., a nsrR-norB-IPA biosynthesis construct) in order to assess nitrogen dependent induction of IPA production. Overnight bacterial cultures are diluted 1:100 into fresh LB and grown for 1.5 hrs to allow entry into early log phase. At this point, long half-life nitric oxide donor (DETA-NO; diethylenetriamine-nitric oxide adduct) w is added to cultures at a final concentration of 0.3 mM to induce expression from plasmid. After 2 hours of induction, cells are spun down, supernatant is discarded, and the cells are resuspended in M9 minimal media containing 0.5% glucose. Culture supernatant is then analyzed at predetermined time points (0 up to 24 hours, as shown in FIG. 33) to assess IPA levels.

In alternate embodiments, production of IPA is also assessed in E. coli Nissle strains containing the IPA and tryptophan cassettes both driven by the low oxygen inducible FNR promoter, e.g., FNRS, or the the reactive oxygene regulated OxyS promoter.

### Example 67. Synthesis of Constructs for Tryptophan Biosynthesis and Indole Metabolite Synthesis

Various constructs are synthesized, and cloned into vector pBR322 for transformation of E. coli. In some embodiments, the constructs encoding the effector molecules are integrated into the genome.

TABLE XXX
Exemplary Sequences and Construct Sequences ofr Tryptophan and Indole
Metabolite Synthesis

| Description | Sequence |
|---|---|
| fbrAroG (RBS and | Ctctagaaataattttgtttaactttaagaaggagatatacat |
| leader region | atgaattatcagaacgacgatttacgcatcaaagaaatcaaagagttacttcctcctgtcgcattgctgga |
| underlined) | aaaattccccgctactgaaaatgccgcgaatacggtcgcccatgcccgaaaagcgatccataagatcct |
| SEQ ID NO: 252 | gaaaggtaatgatgatcgcctgttggtggtgattggcccatgctcaattcatgatcctgtcgcggctaaag |
| | agtatgccactcgcttgctgacgctgcgtgaagagctgcaagatgagctggaaatcgtgatgcgcgtct |
| | attttgaaaagccgcgtactacggtgggctggaaagggctgattaacgatccgcatatggataacagctt |
| | ccagatcaacgacggtctgcgtattgcccgcaaattgctgctcgatattaacgacagcggtctgccagc |
| | ggcgggtgaattcctggatatgatcaccctacaatatctcgctgacctgatgagctggggcgcaattggc |
| | gcacgtaccaccgaatcgcaggtgcaccgcgaactggcgtctggtctttcttgtccggtaggtttcaaaa |
| | atggcactgatggtacgattaaagtggctatcgatgccattaatgccgccggtgcgccgcactgcttcct |
| | gtccgtaacgaaatgggggcattcggcgattgtgaataccagcggtaacggcgattgccatatcattctg |
| | cgcggcggtaaagagcctaactacagcgcgaagcacgttgctgaagtgaaagaagggctgaacaaa |
| | gcaggcctgccagcgcaggtgatgatcgatttcagccatgctaactcgtcaaaacaattcaaaaagcag |
| | atggatgtttgtactgacgtttgccagcagattgccggtggcgaaaaggccattattggcgtgatggtgg |
| | aaagccatctggtggaaggcaatcagagcctcgagagcggggaaccgctggcctacggtaagagca |
| | tcaccgatgcctgcattggctgggatgataccgatgctctgttacgtcaactggcgagtgcagtaaaagc |
| | gcgtcgcgggtaa |
| fbrAroG | atgaattatcagaacgacgatttacgcatcaaagaaatcaaagagttacttcctcctgtcgcattgctgga |
| SEQ ID NO: 258 | aaaattccccgctactgaaaatgccgcgaatacggtcgcccatgcccgaaaagcgatccataagatcct |
| | gaaaggtaatgatgatcgcctgttggtggtgattggcccatgctcaattcatgatcctgtcgcggctaaag |
| | agtatgccactcgcttgctgacgctgcgtgaagagctgcaagatgagctggaaatcgtgatgcgcgtct |
| | attttgaaaagccgcgtactacggtgggctggaaagggctgattaacgatccgcatatggataacagctt |
| | ccagatcaacgacggtctgcgtattgcccgcaaattgctgctcgatattaacgacagcggtctgccagc |
| | ggcgggtgaattcctggatatgatcaccctacaatatctcgctgacctgatgagctggggcgcaattggc |
| | gcacgtaccaccgaatcgcaggtgcaccgcgaactggcgtctggtctttcttgtccggtaggtttcaaaa |
| | atggcactgatggtacgattaaagtggctatcgatgccattaatgccgccggtgcgccgcactgcttcct |
| | gtccgtaacgaaatgggggcattcggcgattgtgaataccagcggtaacggcgattgccatatcattctg |
| | cgcggcggtaaagagcctaactacagcgcgaagcacgttgctgaagtgaaagaagggctgaacaaa |
| | gcaggcctgccagcgcaggtgatgatcgatttcagccatgctaactcgtcaaaacaattcaaaaagcag |
| | atggatgtttgtactgacgtttgccagcagattgccggtggcgaaaaggccattattggcgtgatggtgg |
| | aaagccatctggtggaaggcaatcagagcctcgagagcggggaaccgctggcctacggtaagagca |
| | tcaccgatgcctgcattggctgggatgataccgatgctctgttacgtcaactggcgagtgcagtaaaagc |
| | gcgtcgcgggtaa |
| fbr-AroG-serA | Ctctagaaataattttgtttaactttaagaaggagatatacatatgaattatcagaacgacgatttacgcatc |
| (RBS and leader | aaagaaatcaaagagttacttcctcctgtcgcattgctggaaaaattccccgctactgaaaatgccgcga |
| region underlined; | atacggtcgcccatgcccgaaaagcgatccataagatcctgaaaggtaatgatgatcgcctgttggtgg |
| SerA starts after | tgattggcccatgctcaattcatgatcctgtcgcggctaaagagtatgccactcgcttgctgacgctgcgt |
| second RBS) | gaagagctgcaagatgagctggaaatcgtgatgcgcgtctattttgaaaagccgcgtactacggtgggc |
| SEQ ID NO: 253 | tggaaagggctgattaacgatccgcatatggataacagcttccagatcaacgacggtctgcgtattgccc |
| | gcaaattgctgctcgatattaacgacagcggtctgccagcggcgggtgaattcctggatatgatcaccct |
| | acaatatctcgctgacctgatgagctggggcgcaattggcgcacgtaccaccgaatcgcaggtgcacc |
| | gcgaactggcgtctggtctttcttgtccggtaggtttcaaaaatggcactgatggtacgattaaagtggct |
| | atcgatgccattaatgccgccggtgcgccgcactgcttcctgtccgtaacgaaatgggggcattcggcg |
| | attgtgaataccagcggtaacggcgattgccatatcattctgcgcggcggtaaagagcctaactacagc |
| | gcgaagcacgttgctgaagtgaaagaagggctgaacaaagcaggcctgccagcgcaggtgatgatc |
| | gatttcagccatgctaactcgtcaaaacaattcaaaaagcagatggatgtttgtactgacgtttgccagca |
| | gattgccggtggcgaaaaggccattattggcgtgatggtggaaagccatctggtggaaggcaatcaga |
| | gcctcgagagcggggaaccgctggcctacggtaagagcatcaccgatgcctgcattggctgggatga |
| | taccgatgctctgttacgtcaactggcgagtgcagtaaaagcgcgtcgcgggtaaTACT |
| | taagaaggagatatacatatggcaaaggtatcgctggagaaagacaagattaagtttctgctggtagaa |
| | ggcgtgcaccaaaaggcgctggaaagccttcgtgcagctggttacaccaacatcgaatttcacaaagg |
| | cgcgctggatgatgaacaattaaaagaatccatccgcgatgcccacttcatcggcctgcgatcccgtac |
| | ccatctgactgaagacgtgatcaacgccgcagaaaaactggtcgctattggctgtttctgtatcggaaca |
| | aatcaggttgatctggatgcggcggcaaagcgcgggatcccggtatttaacgcaccgttctcaaatacg |
| | cgctctgttgcggagctggtgattggcgaactgctgctgctattgcgcggcgtgccagaagccaatgct |
| | aaagcgcatcgtggcgtgtggaacaaactggcggcgggttcttttgaagcgcgcggcaaaaagctgg |
| | gtatcatcggctacggtcatattggtacgcaattgggcattctggctgaatcgctgggaatgtatgtttactt |
| | ttatgatattgaaaacaaactgccgctgggcaacgccactcaggtacagcatctttctgacctgctgaata |
| | tgagcgatgtggtgagtctgcatgtaccagagaatccgtccaccaaaaatatgatgggcgcgaaagag |
| | atttcgctaatgaagcccggctcgctgctgattaatgcttcgcgcggtactgtggtggatattccagcgct |
| | gtgtgacgcgctggcgagcaaacatctggcgggggcggcaatcgacgtattcccgacggaaccggc |
| | gaccaatagcgatccatttacctctccgctgtgtgaattcgacaatgtccttctgacgccacacattggcg |
| | gttcgactcaggaagcgcaggagaatatcggcttggaagttgcgggtaaattgatcaagtattctgacaa |
| | tggctcaacgctctctgcggtgaacttcccggaagtctcgctgccactgcacggtgggcgtcgtctgat |
| | gcacatccacgaaaaccgtccgggcgtgctaactgcgctcaacaaaatttttgccgagcagggcgtca |
| | acatcgccgcgcaatatctacaaacttccgcccagatgggttatgtagttattgatattgaagccgacgaa |
| | gacgttgccgaaaaagcgctgcaggcaatgaaagctattccgggtaccattcgcgcccgtctgctgtac |
| | taa |
| SerA (RBS | atggcaaaggtatcgctggagaaagacaagattaagtttctgctggtagaaggcgtgcaccaaaaggc |
| underlined) | gctggaaagccttcgtgcagctggttacaccaacatcgaatttcacaaaggcgcgctggatgatgaaca |
| SEQ ID NO: 254 | attaaaagaatccatccgcgatgcccacttcatcggcctgcgatcccgtacccatctgactgaagacgtg |
| | atcaacgccgcagaaaaactggtcgctattggctgtttctgtatcggaacaaatcaggttgatctggatgc |
| | ggcggcaaagcgcgggatcccggtatttaacgcaccgttctcaaatacgcgctctgttgcggagctggt |
| | gattggcgaactgctgctgctattgcgcggcgtgccagaagccaatgctaaagcgcatcgtggcgtgt |
| | ggaacaaactggcggcgggttcttttgaagcgcgcggcaaaaagctgggtatcatcggctacggtcat |
| | attggtacgcaattgggcattctggctgaatcgctgggaatgtatgtttacttttatgatattgaaaacaaac |
| | tgccgctgggcaacgccactcaggtacagcatctttctgacctgctgaatatgagcgatgtggtgagtct |
| | gcatgtaccagagaatccgtccaccaaaaatatgatgggcgcgaaagagatttcgctaatgaagcccg |
| | gctcgctgctgattaatgcttcgcgcggtactgtggtggatattccagcgctgtgtgacgcgctggcgag |
| | caaacatctggcgggggcggcaatcgacgtattcccgacggaaccggcgaccaatagcgatccattta |
| | cctctccgctgtgtgaattcgacaatgtccttctgacgccacacattggcggttcgactcaggaagcgca |
| | ggagaatatcggcttggaagttgcgggtaaattgatcaagtattctgacaatggctcaacgctctctgcg |
| | gtgaacttcccggaagtctcgctgccactgcacggtgggcgtcgtctgatgcacatccacgaaaaccgt |
| | ccgggcgtgctaactgcgctcaacaaaatttttgccgagcagggcgtcaacatcgccgcgcaatatcta |
| | caaacttccgcccagatgggttatgtagttattgatattgaagccgacgaagacgttgccgaaaaagcgc |
| | tgcaggcaatgaaagctattccgggtaccattcgcgcccgtctgctgtactaa |
| fbrAroG-Tdc (tdc | ctctagaaataattttgtttaactttaagaaggagatatacatatgaattatcagaacgacgatttacgcatc |
| from C. roseus); | aaagaaatcaaagagttacttcctcctgtcgcattgctggaaaaattccccgctactgaaaatgccgcga |
| RBS and leader | atacggtcgcccatgcccgaaaagcgatccataagatcctgaaaggtaatgatgatcgcctgttggtgg |
| region underlined | tgattggcccatgctcaattcatgatcctgtcgcggctaaagagtatgccactcgcttgctgacgctgcgt |
| SEQ ID NO: 255 | gaagagctgcaagatgagctggaaatcgtgatgcgcgtctattttgaaaagccgcgtactacggtgggc |
| | tggaaagggctgattaacgatccgcatatggataacagcttccagatcaacgacggtctgcgtattgccc |
| | gcaaattgctgctcgatattaacgacagcggtctgccagcggcgggtgaattcctggatatgatcaccct |
| | acaatatctcgctgacctgatgagctggggcgcaattggcgcacgtaccaccgaatcgcaggtgcacc |
| | gcgaactggcgtctggtctttcttgtccggtaggtttcaaaaatggcactgatggtacgattaaagtggct |
| | atcgatgccattaatgccgccggtgcgccgcactgcttcctgtccgtaacgaaatgggggcattcggcg |
| | attgtgaataccagcggtaacggcgattgccatatcattctgcgcggcggtaaagagcctaactacagc |
| | gcgaagcacgttgctgaagtgaaagaagggctgaacaaagcaggcctgccagcgcaggtgatgatc |
| | gatttcagccatgctaactcgtcaaaacaattcaaaaagcagatggatgtttgtactgacgtttgccagca |
| | gattgccggtggcgaaaaggccattattggcgtgatggtggaaagccatctggtggaaggcaatcaga |
| | gcctcgagagcggggaaccgctggcctacggtaagagcatcaccgatgcctgcattggctgggatga |
| | taccgatgctctgttacgtcaactggcgagtgcagtaaaagcgcgtcgcgggtaaTACTtaagaag |
| | gagatatacatATGGGTTCTATTGACTCGACGAATGTGGCCATGTCT |
| | AATTCTCCTGTTGGCGAGTTTAAGCCCCTTGAAGCAGAAGAGT |
| | TCCGTAAACAGGCACACCGCATGGTGGATTTTATTGCGGATTA |
| | TTACAAGAACGTAGAAACATACCCGGTCCTTTCCGAGGTTGAA |
| | CCCGGCTATCTGCGCAAACGTATTCCCGAAACCGCACCATACC |
| | TGCCGGAGCCACTTGATGATATTATGAAGGATATTCAAAAGG |
| | ACATTATCCCCGGAATGACGAACTGGATGTCCCCGAACTTTTA |
| | CGCCTTCTTCCCGGCCACAGTTAGCTCAGCAGCTTTCTTGGGG |
| | GAAATGCTTTCAACGGCCCTTAACAGCGTAGGATTTACCTGGG |
| | TCAGTTCCCCGGCAGCGACTGAATTAGAGATGATCGTTATGGA |
| | TTGGCTTGCGCAAATTTTGAAACTTCCAAAAAGCTTTATGTTCT |
| | CCGGAACCGGGGGTGGTGTCATCCAAAACACTACGTCAGAGT |
| | CGATCTTGTGCACTATTATCGCGGCCCGTGAACGCGCCTTGGA |
| | AAAATTGGGCCCTGATTCAATTGGTAAGCTTGTCTGCTATGGG |
| | TCCGATCAAACGCACACAATGTTTCCGAAAACCTGTAAGTTAG |
| | CAGGAATTTATCCGAATAATATCCGCCTTATCCCTACCACGGT |
| | AGAAACCGACTTTGGCATCTCACCGCAGGTACTTCGCAAGATG |
| | GTCGAAGACGACGTCGCTGCGGGGTACGTTCCCTTATTTTTGT |
| | GTGCCACCTTGGGAACGACATCAACTACGGCAACAGATCCTGT |
| | AGATTCGCTGTCCGAAATCGCAAACGAGTTTGGTATCTGGATT |
| | CATGTCGACGCCGCATATGCTGGATCGGCTTGCATCTGCCCAG |
| | AATTTCGTCACTACCTTGATGGCATCGAACGTGTGGATTCCTT |
| | ATCGCTGTCTCCCCACAAATGGCTTTTAGCATATCTGGATTGC |
| | ACGTGCTTGTGGGTAAAACAACCTCACCTGCTGCTTCGCGCTT |
| | TAACGACTAATCCCGAATACTTGAAGAATAAACAGAGTGATTT |
| | AGATAAGGTCGTGGATTTTAAGAACTGGCAGATCGCAACAGG |
| | ACGTAAGTTCCGCTCTTTAAAACTTTGGTTAATTCTGCGTTCCT |
| | ACGGGGTAGTTAACCTGCAAAGTCATATCCGTAGTGATGTAGC |
| | GATGGGGAAGATGTTTGAGGAATGGGTCCGTTCCGATAGCCG |
| | CTTTGAAATCGTCGTGCCACGTAATTTTTCGCTTGTATGCTTTC |
| | GCTTGAAACCGGATGTATCTAGTTTACATGTCGAGGAGGTCAA |
| | CAAGAAGTTGTTGGATATGCTTAACTCCACCGGTCGCGTATAT |
| | ATGACGCATACAATTGTTGGCGGAATCTATATGTTACGTTTGG |
| | CTGTAGGTAGCAGCTTGACAGAGGAACATCACGTGCGCCGCG |
| | TTTGGGACTTGATCCAGAAGCTTACGGACGACCTGCTTAAAGA |
| | GGCGTGA |
| Tdc (tdc from | ATGGGTTCTATTGACTCGACGAATGTGGCCATGTCTAATTCTC |
| C. roseus) | CTGTTGGCGAGTTTAAGCCCCTTGAAGCAGAAGAGTTCCGTAA |
| SEQ ID NO: 256 | ACAGGCACACCGCATGGTGGATTTTATTGCGGATTATTACAAG |
| | AACGTAGAAACATACCCGGTCCTTTCCGAGGTTGAACCCGGCT |
| | ATCTGCGCAAACGTATTCCCGAAACCGCACCATACCTGCCGGA |
| | GCCACTTGATGATATTATGAAGGATATTCAAAAGGACATTATC |
| | CCCGGAATGACGAACTGGATGTCCCCGAACTTTTACGCCTTCT |
| | TCCCGGCCACAGTTAGCTCAGCAGCTTTCTTGGGGGAAATGCT |
| | TTCAACGGCCCTTAACAGCGTAGGATTTACCTGGGTCAGTTCC |
| | CCGGCAGCGACTGAATTAGAGATGATCGTTATGGATTGGCTTG |
| | CGCAAATTTTGAAACTTCCAAAAAGCTTTATGTTCTCCGGAAC |
| | CGGGGGTGGTGTCATCCAAAACACTACGTCAGAGTCGATCTTG |
| | TGCACTATTATCGCGGCCCGTGAACGCGCCTTGGAAAAATTGG |
| | GCCCTGATTCAATTGGTAAGCTTGTCTGCTATGGGTCCGATCA |
| | AACGCACACAATGTTTCCGAAAACCTGTAAGTTAGCAGGAATT |
| | TATCCGAATAATATCCGCCTTATCCCTACCACGGTAGAAACCG |
| | ACTTTGGCATCTCACCGCAGGTACTTCGCAAGATGGTCGAAGA |
| | CGACGTCGCTGCGGGGTACGTTCCCTTATTTTTGTGTGCCACCT |
| | TGGGAACGACATCAACTACGGCAACAGATCCTGTAGATTCGCT |
| | GTCCGAAATCGCAAACGAGTTTGGTATCTGGATTCATGTCGAC |
| | GCCGCATATGCTGGATCGGCTTGCATCTGCCCAGAATTTCGTC |
| | ACTACCTTGATGGCATCGAACGTGTGGATTCCTTATCGCTGTC |
| | TCCCCACAAATGGCTTTTAGCATATCTGGATTGCACGTGCTTG |
| | TGGGTAAAACAACCTCACCTGCTGCTTCGCGCTTTAACGACTA |
| | ATCCCGAATACTTGAAGAATAAACAGAGTGATTTAGATAAGG |
| | TCGTGGATTTTAAGAACTGGCAGATCGCAACAGGACGTAAGTT |
| | CCGCTCTTTAAAACTTTGGTTAATTCTGCGTTCCTACGGGGTAG |
| | TTAACCTGCAAAGTCATATCCGTAGTGATGTAGCGATGGGGAA |
| | GATGTTTGAGGAATGGGTCCGTTCCGATAGCCGCTTTGAAATC |
| | GTCGTGCCACGTAATTTTTCGCTTGTATGCTTTCGCTTGAAACC |
| | GGATGTATCTAGTTTACATGTCGAGGAGGTCAACAAGAAGTTG |
| | TTGGATATGCTTAACTCCACCGGTCGCGTATATATGACGCATA |
| | CAATTGTTGGCGGAATCTATATGTTACGTTTGGCTGTAGGTAG |
| | CAGCTTGACAGAGGAACATCACGTGCGCCGCGTTTGGGACTTG |
| | ATCCAGAAGCTTACGGACGACCTGCTTAAAGAGGCGTGA |
| fbrAroG-Tdc (tdc | ctctagaaataattttgtttaactttaagaaggagatatacatatgaattatcagaacgacgatttacgcatc |
| from Clostridium | aaagaaatcaaagagttacttcctcctgtcgcattgctggaaaaattccccgctactgaaaatgccgcga |
| sporogenes); RBS | atacggtcgcccatgcccgaaaagcgatccataagatcctgaaaggtaatgatgatcgcctgttggtgg |
| and leader region | tgattggcccatgctcaattcatgatcctgtcgcggctaaagagtatgccactcgcttgctgacgctgcgt |
| underlined | gaagagctgcaagatgagctggaaatcgtgatgcgcgtctattttgaaaagccgcgtactacggtgggc |
| SEQ ID NO: | tggaaagggctgattaacgatccgcatatggataacagcttccagatcaacgacggtctgcgtattgccc |
| XXXX | gcaaattgctgctcgatattaacgacagcggtctgccagcggcgggtgaattcctggatatgatcaccct |
| | acaatatctcgctgacctgatgagctggggcgcaattggcgcacgtaccaccgaatcgcaggtgcacc |
| | gcgaactggcgtctggtctttcttgtccggtaggtttcaaaaatggcactgatggtacgattaaagtggct |
| | atcgatgccattaatgccgccggtgcgccgcactgcttcctgtccgtaacgaaatgggggcattcggcg |
| | attgtgaataccagcggtaacggcgattgccatatcattctgcgcggcggtaaagagcctaactacagc |
| | gcgaagcacgttgctgaagtgaaagaagggctgaacaaagcaggcctgccagcgcaggtgatgatc |
| | gatttcagccatgctaactcgtcaaaacaattcaaaaagcagatggatgtttgtactgacgtttgccagca |
| | gattgccggtggcgaaaaggccattattggcgtgatggtggaaagccatctggtggaaggcaatcaga |
| | gcctcgagagcggggaaccgctggcctacggtaagagcatcaccgatgcctgcattggctgggatga |
| | taccgatgctctgttacgtcaactggcgagtgcagtaaaagcgcgtcgcgggtaaTACTtaagaag |
| | gagatatacatATGAAATTTTGGCGCAAGTATACGCAACAGGAGATG |
| | GATGAGAAAATCACAGAATCGCTTGAGAAGACATTAAATTAC |
| | GATAACACGAAAACCATCGGCATCCCAGGTACTAAGCTGGAT |
| | GATACTGTATTTTATGACGATCACTCCTTCGTTAAGCACTCTCC |
| | CTATTTACGTACGTTCATCCAAAACCCTAATCACATTGGTTGTC |
| | ACACGTACGATAAAGCAGACATCTTGTTTGGCGGCACGTTTGA |
| | CATCGAACGCGAACTGATTCAGCTTTTGGCCATCGATGTCTTA |
| | AACGGAAATGATGAGGAATTCGATGGATATGTGACACAGGGG |
| | GGAACCGAGGCGAATATTCAGGCAATGTGGGTTTATCGTAACT |
| | ATTTCAAAAAAGAACGTAAAGCAAAACATGAGGAAATCGCAA |
| | TCATCACGAGCGCGGATACCCATTACAGTGCATATAAGGGGA |
| | GCGACTTGCTGAACATTGATATTATCAAGGTCCCAGTAGACTT |
| | CTATTCGCGTAAGATCCAGGAGAACACGTTAGACTCGATTGTC |
| | AAGGAGGCGAAGGAAATTGGAAAGAAGTACTTCATTGTCATC |
| | TCAAACATGGGTACGACTATGTTTGGCAGTGTAGACGACCCTG |
| | ATCTTTATGCTAACATTTTTGATAAGTATAACTTAGAATACAA |
| | AATCCACGTCGATGGAGCTTTTGGGGGTTTCATTTATCCTATC |
| | GATAATAAGGAGTGCAAAACAGATTTCTCGAACAAGAACGTC |
| | TCATCCATCACGCTTGACGGTCACAAAATGCTTCAAGCCCCCT |
| | ATGGGACTGGTATCTTCGTGTCACGTAAGAACTTGATCCATAA |
| | CACCCTGACAAAGGAAGCAACGTATATTGAAAACCTGGACGT |
| | TACCCTGAGTGGGTCCCGCTCCGGATCCAACGCCGTTGCGATC |
| | TGGATGGTTTTAGCCTCTTATGGCCCCTACGGGTGGATGGAGA |
| | AGATTAACAAGTTGCGCAATCGCACTAAGTGGCTTTGCAAGCA |
| | GCTTAACGACATGCGCATCAAATACTATAAGGAGGATAGCAT |
| | GAATATCGTCACGATTGAAGAGCAATACGTAAATAAAGAGAT |
| | TGCAGAGAAATACTTCCTTGTGCCTGAAGTACACAATCCTACC |
| | AACAATTGGTACAAGATTGTAGTCATGGAACATGTTGAACTTG |
| | ACATCTTGAACTCCCTTGTTTATGATTTACGTAAATTCAACAA |
| | GGAGCACCTGAAGGCAATGTGA |
| Tdc (tdc from | ATGAAATTTTGGCGCAAGTATACGCAACAGGAGATGGATGAG |
| Clostridium | AAAATCACAGAATCGCTTGAGAAGACATTAAATTACGATAAC |
| sporogenes) | ACGAAAACCATCGGCATCCCAGGTACTAAGCTGGATGATACT |
| SEQ ID NO: | GTATTTTATGACGATCACTCCTTCGTTAAGCACTCTCCCTATTT |
| XXXX | ACGTACGTTCATCCAAAACCCTAATCACATTGGTTGTCACACG |
| | TACGATAAAGCAGACATCTTGTTTGGCGGCACGTTTGACATCG |
| | AACGCGAACTGATTCAGCTTTTGGCCATCGATGTCTTAAACGG |
| | AAATGATGAGGAATTCGATGGATATGTGACACAGGGGGGAAC |
| | CGAGGCGAATATTCAGGCAATGTGGGTTTATCGTAACTATTTC |
| | AAAAAAGAACGTAAAGCAAAACATGAGGAAATCGCAATCATC |
| | ACGAGCGCGGATACCCATTACAGTGCATATAAGGGGAGCGAC |
| | TTGCTGAACATTGATATTATCAAGGTCCCAGTAGACTTCTATT |
| | CGCGTAAGATCCAGGAGAACACGTTAGACTCGATTGTCAAGG |
| | AGGCGAAGGAAATTGGAAAGAAGTACTTCATTGTCATCTCAA |
| | ACATGGGTACGACTATGTTTGGCAGTGTAGACGACCCTGATCT |
| | TTATGCTAACATTTTTGATAAGTATAACTTAGAATACAAAATC |
| | CACGTCGATGGAGCTTTTGGGGGTTTCATTTATCCTATCGATA |
| | ATAAGGAGTGCAAAACAGATTTCTCGAACAAGAACGTCTCAT |
| | CCATCACGCTTGACGGTCACAAAATGCTTCAAGCCCCCTATGG |
| | GACTGGTATCTTCGTGTCACGTAAGAACTTGATCCATAACACC |
| | CTGACAAAGGAAGCAACGTATATTGAAAACCTGGACGTTACC |
| | CTGAGTGGGTCCCGCTCCGGATCCAACGCCGTTGCGATCTGGA |
| | TGGTTTTAGCCTCTTATGGCCCCTACGGGTGGATGGAGAAGAT |
| | TAACAAGTTGCGCAATCGCACTAAGTGGCTTTGCAAGCAGCTT |
| | AACGACATGCGCATCAAATACTATAAGGAGGATAGCATGAAT |
| | ATCGTCACGATTGAAGAGCAATACGTAAATAAAGAGATTGCA |
| | GAGAAATACTTCCTTGTGCCTGAAGTACACAATCCTACCAACA |
| | ATTGGTACAAGATTGTAGTCATGGAACATGTTGAACTTGACAT |
| | CTTGAACTCCCTTGTTTATGATTTACGTAAATTCAACAAGGAG |
| | CACCTGAAGGCAATGTGA |
| fbrAroG-trpDH- | Ctctagaaataattttgtttaactttaagaaggagatatacat |
| ipdC-iad1 (RBS | atgaattatcagaacgacgatttacgcatcaaagaaatcaaagagttacttcctcctgtcgcattgctgga |
| and leader region | aaaattccccgctactgaaaatgccgcgaatacggtcgcccatgcccgaaaagcgatccataagatcct |
| underlined) | gaaaggtaatgatgatcgcctgttggtggtgattggcccatgctcaattcatgatcctgtcgcggctaaag |
| SEQ ID NO: 257 | agtatgccactcgcttgctgacgctgcgtgaagagctgcaagatgagctggaaatcgtgatgcgcgtct |
| | attttgaaaagccgcgtactacggtgggctggaaagggctgattaacgatccgcatatggataacagctt |
| | ccagatcaacgacggtctgcgtattgcccgcaaattgctgctcgatattaacgacagcggtctgccagc |
| | ggcgggtgaattcctggatatgatcaccctacaatatctcgctgacctgatgagctggggcgcaattggc |
| | gcacgtaccaccgaatcgcaggtgcaccgcgaactggcgtctggtctttcttgtccggtaggtttcaaaa |
| | atggcactgatggtacgattaaagtggctatcgatgccattaatgccgccggtgcgccgcactgcttcct |
| | gtccgtaacgaaatgggggcattcggcgattgtgaataccagcggtaacggcgattgccatatcattctg |
| | cgcggcggtaaagagcctaactacagcgcgaagcacgttgctgaagtgaaagaagggctgaacaaa |
| | gcaggcctgccagcgcaggtgatgatcgatttcagccatgctaactcgtcaaaacaattcaaaaagcag |
| | atggatgtttgtactgacgtttgccagcagattgccggtggcgaaaaggccattattggcgtgatggtgg |
| | aaagccatctggtggaaggcaatcagagcctcgagagcggggaaccgctggcctacggtaagagca |
| | tcaccgatgcctgcattggctgggatgataccgatgctctgttacgtcaactggcgagtgcagtaaaagc |
| | gcgtcgcgggtaaTACTtaagaaggagatatacatATGCTGTTATTCGAGACTGT |
| | GCGTGAAATGGGTCATGAGCAAGTCCTTTTCTGTCATAGCAAG |
| | AATCCCGAGATCAAGGCAATTATCGCAATCCACGATACCACCT |
| | TAGGACCGGCTATGGGCGCAACTCGTATCTTACCTTATATTAA |
| | TGAGGAGGCTGCCCTGAAAGATGCATTACGTCTGTCCCGCGGA |
| | ATGACTTACAAAGCAGCCTGCGCCAATATTCCCGCCGGGGGC |
| | GGCAAAGCCGTCATCATCGCTAACCCCGAAAACAAGACCGAT |
| | GACCTGTTACGCGCATACGGCCGTTTCGTGGACAGCTTGAACG |
| | GCCGTTTCATCACCGGGCAGGACGTTAACATTACGCCCGACGA |
| | CGTTCGCACTATTTCGCAGGAGACTAAGTACGTGGTAGGCGTC |
| | TCAGAAAAGTCGGGAGGGCCGGCACCTATCACCTCTCTGGGA |
| | GTATTTTTAGGCATCAAAGCCGCTGTAGAGTCGCGTTGGCAGT |
| | CTAAACGCCTGGATGGCATGAAAGTGGCGGTGCAAGGACTTG |
| | GGAACGTAGGAAAAAATCTTTGTCGCCATCTGCATGAACACG |
| | ATGTACAACTTTTTGTGTCTGATGTCGATCCAATCAAGGCCGA |
| | GGAAGTAAAACGCTTATTCGGGGCGACTGTTGTCGAACCGACT |
| | GAAATCTATTCTTTAGATGTTGATATTTTTGCACCGTGTGCACT |
| | TGGGGGTATTTTGAATAGCCATACCATCCCGTTCTTACAAGCC |
| | TCAATCATCGCAGGAGCAGCGAATAACCAGCTGGAGAACGAG |
| | CAACTTCATTCGCAGATGCTTGCGAAAAAGGGTATTCTTTACT |
| | CACCAGACTACGTTATCAATGCAGGAGGACTTATCAATGTTTA |
| | TAACGAAATGATCGGATATGACGAGGAAAAAGCATTCAAACA |
| | AGTTCATAACATCTACGATACGTTATTAGCGATTTTCGAAATT |
| | GCAAAAGAACAAGGTGTAACCACCAACGACGCGGCCCGTCGT |
| | TTAGCAGAGGATCGTATCAACAACTCCAAACGCTCAAAGAGT |
| | AAAGCGATTGCGGCGTGAAATGtaagaaggagatatacatATGCGTACA |
| | CCCTACTGTGTCGCCGATTATCTTTTAGATCGTCTGACGGACTG |
| | CGGGGCCGATCACCTGTTTGGCGTACCGGGCGATTACAACTTG |
| | CAGTTTCTGGACCACGTCATTGACTCACCAGATATCTGCTGGG |
| | TAGGGTGTGCGAACGAGCTTAACGCGAGCTACGCTGCTGACG |
| | GATATGCGCGTTGTAAAGGCTTTGCTGCACTTCTTACTACCTTC |
| | GGGGTCGGTGAGTTATCGGCGATGAACGGTATCGCAGGCTCG |
| | TACGCTGAGCACGTCCCGGTATTACACATTGTGGGAGCTCCGG |
| | GTACCGCAGCTCAACAGCGCGGAGAACTGTTACACCACACGC |
| | TGGGCGACGGAGAATTCCGCCACTTTTACCATATGTCCGAGCC |
| | AATTACTGTAGCCCAGGCTGTACTTACAGAGCAAAATGCCTGT |
| | TACGAGATCGACCGTGTTTTGACCACGATGCTTCGCGAGCGCC |
| | GTCCCGGGTATTTGATGCTGCCAGCCGATGTTGCCAAAAAAGC |
| | TGCGACGCCCCCAGTGAATGCCCTGACGCATAAACAAGCTCAT |
| | GCCGATTCCGCCTGTTTAAAGGCTTTTCGCGATGCAGCTGAAA |
| | ATAAATTAGCCATGTCGAAACGCACCGCCTTGTTGGCGGACTT |
| | TCTGGTCCTGCGCCATGGCCTTAAACACGCCCTTCAGAAATGG |
| | GTCAAAGAAGTCCCGATGGCCCACGCTACGATGCTTATGGGTA |
| | AGGGGATTTTTGATGAACGTCAAGCGGGATTTTATGGAACTTA |
| | TTCCGGTTCGGCGAGTACGGGGGCGGTAAAGGAAGCGATTGA |
| | GGGAGCCGACACAGTTCTTTGCGTGGGGACACGTTTCACCGAT |
| | ACACTGACCGCTGGATTCACACACCAACTTACTCCGGCACAAA |
| | CGATTGAGGTGCAACCCCATGCGGCTCGCGTGGGGGATGTAT |
| | GGTTTACGGGCATTCCAATGAATCAAGCCATTGAGACTCTTGT |
| | CGAGCTGTGCAAACAGCACGTCCACGCAGGACTGATGAGTTC |
| | GAGCTCTGGGGCGATTCCTTTTCCACAACCAGATGGTAGTTTA |
| | ACTCAAGAAAACTTCTGGCGCACATTGCAAACCTTTATCCGCC |
| | CAGGTGATATCATCTTAGCAGACCAGGGTACTTCAGCCTTTGG |
| | AGCAATTGACCTGCGCTTACCAGCAGACGTGAACTTTATTGTG |
| | CAGCCGCTGTGGGGGTCTATTGGTTATACTTTAGCTGCGGCCT |
| | TCGGAGCGCAGACAGCGTGTCCAAACCGTCGTGTGATCGTATT |
| | GACAGGAGATGGAGCAGCGCAGTTGACCATTCAGGAGTTAGG |
| | CTCGATGTTACGCGATAAGCAGCACCCCATTATCCTGGTCCTG |
| | AACAATGAGGGGTATACAGTTGAACGCGCCATTCATGGTGCG |
| | GAACAACGCTACAATGACATCGCTTTATGGAATTGGACGCAC |
| | ATCCCCCAAGCCTTATCGTTAGATCCCCAATCGGAATGTTGGC |
| | GTGTGTCTGAAGCAGAGCAACTGGCTGATGTTCTGGAAAAAG |
| | TTGCTCATCATGAACGCCTGTCGTTGATCGAGGTAATGTTGCC |
| | CAAGGCCGATATCCCTCCGTTACTGGGAGCCTTGACCAAGGCT |
| | TTAGAAGCCTGCAACAACGCTTAAAGGTtaagaaggagatatacatATG |
| | CCCACCTTGAACTTGGACTTACCCAACGGTATTAAGAGCACGA |
| | TTCAGGCAGACCTTTTCATCAATAATAAGTTTGTGCCGGCGCT |
| | TGATGGGAAAACGTTCGCAACTATTAATCCGTCTACGGGGAA |
| | AGAGATCGGACAGGTGGCAGAGGCTTCGGCGAAGGATGTGGA |
| | TCTTGCAGTTAAGGCCGCGCGTGAGGCGTTTGAAACTACTTGG |
| | GGGGAAAACACGCCAGGTGATGCTCGTGGCCGTTTACTGATTA |
| | AGCTTGCTGAGTTGGTGGAAGCGAATATTGATGAGTTAGCGGC |
| | AATTGAATCACTGGACAATGGGAAAGCGTTCTCTATTGCTAAG |
| | TCATTCGACGTAGCTGCTGTGGCCGCAAACTTACGTTACTACG |
| | GCGGTTGGGCTGATAAAAACCACGGTAAAGTCATGGAGGTAG |
| | ACACAAAGCGCCTGAACTATACCCGCCACGAGCCGATCGGGG |
| | TTTGCGGACAAATCATTCCGTGGAATTTCCCGCTTTTGATGTTT |
| | GCATGGAAGCTGGGTCCCGCTTTAGCCACAGGGAACACAATT |
| | GTGTTAAAGACTGCCGAGCAGACTCCCTTAAGTGCTATCAAGA |
| | TGTGTGAATTAATCGTAGAAGCCGGCTTTCCGCCCGGAGTAGT |
| | TAATGTGATCTCGGGATTCGGACCGGTGGCGGGGGCCGCGAT |
| | CTCGCAACACATGGACATCGATAAGATTGCCTTTACAGGATCG |
| | ACATTGGTTGGCCGCAACATTATGAAGGCAGCTGCGTCGACTA |
| | ACTTAAAAAAGGTTACACTTGAGTTAGGAGGAAAATCCCCGA |
| | ATATCATTTTCAAAGATGCCGACCTTGACCAAGCTGTTCGCTG |
| | GAGCGCCTTCGGTATCATGTTTAACCACGGACAATGCTGCTGC |
| | GCTGGATCGCGCGTATATGTGGAAGAATCCATCTATGACGCCT |
| | TCATGGAAAAAATGACTGCGCATTGTAAGGCGCTTCAAGTTGG |
| | AGATCCTTTCAGCGCGAACACCTTCCAAGGACCACAAGTCTCG |
| | CAGTTACAATACGACCGTATCATGGAATACATCGAATCAGGG |
| | AAAAAAGATGCAAATCTTGCTTTAGGCGGCGTTCGCAAAGGG |
| | AATGAGGGGTATTTCATTGAGCCAACTATTTTTACAGACGTGC |
| | CGCACGACGCGAAGATTGCCAAAGAGGAGATCTTCGGTCCAG |
| | TGGTTGTTGTGTCGAAATTTAAGGACGAAAAAGATCTGATCCG |
| | TATCGCAAATGATTCTATTTATGGTTTAGCTGCGGCAGTCTTTT |
| | CCCGCGACATCAGCCGCGCGATCGAGACAGCACACAAACTGA |
| | AAGCAGGCACGGTCTGGGTCAACTGCTATAATCAGCTTATTCC |
| | GCAGGTGCCATTCGGAGGGTATAAGGCTTCCGGTATCGGCCGT |
| | GAGTTGGGGGAATATGCCTTGTCTAATTACACAAATATCAAGG |
| | CCGTCCACGTTAACCTTTCTCAACCGGCGCCCATTTGA |
| trpDH (RBS | Taagaaggagatatacat |
| underlined) | ATGCTGTTATTCGAGACTGTGCGTGAAATGGGTCATGAGCAAG |
| SEQ ID NO: 259 | TCCTTTTCTGTCATAGCAAGAATCCCGAGATCAAGGCAATTAT |
| | CGCAATCCACGATACCACCTTAGGACCGGCTATGGGCGCAACT |
| | CGTATCTTACCTTATATTAATGAGGAGGCTGCCCTGAAAGATG |
| | CATTACGTCTGTCCCGCGGAATGACTTACAAAGCAGCCTGCGC |
| | CAATATTCCCGCCGGGGGCGGCAAAGCCGTCATCATCGCTAAC |
| | CCCGAAAACAAGACCGATGACCTGTTACGCGCATACGGCCGT |
| | TTCGTGGACAGCTTGAACGGCCGTTTCATCACCGGGCAGGACG |
| | TTAACATTACGCCCGACGACGTTCGCACTATTTCGCAGGAGAC |
| | TAAGTACGTGGTAGGCGTCTCAGAAAAGTCGGGAGGGCCGGC |
| | ACCTATCACCTCTCTGGGAGTATTTTTAGGCATCAAAGCCGCT |
| | GTAGAGTCGCGTTGGCAGTCTAAACGCCTGGATGGCATGAAA |
| | GTGGCGGTGCAAGGACTTGGGAACGTAGGAAAAAATCTTTGT |
| | CGCCATCTGCATGAACACGATGTACAACTTTTTGTGTCTGATG |
| | TCGATCCAATCAAGGCCGAGGAAGTAAAACGCTTATTCGGGG |
| | CGACTGTTGTCGAACCGACTGAAATCTATTCTTTAGATGTTGA |
| | TATTTTTGCACCGTGTGCACTTGGGGGTATTTTGAATAGCCAT |
| | ACCATCCCGTTCTTACAAGCCTCAATCATCGCAGGAGCAGCGA |
| | ATAACCAGCTGGAGAACGAGCAACTTCATTCGCAGATGCTTGC |
| | GAAAAAGGGTATTCTTTACTCACCAGACTACGTTATCAATGCA |
| | GGAGGACTTATCAATGTTTATAACGAAATGATCGGATATGACG |
| | AGGAAAAAGCATTCAAACAAGTTCATAACATCTACGATACGT |
| | TATTAGCGATTTTCGAAATTGCAAAAGAACAAGGTGTAACCAC |
| | CAACGACGCGGCCCGTCGTTTAGCAGAGGATCGTATCAACAA |
| | CTCCAAACGCTCAAAGAGTAAAGCGATTGCGGCGTGA |
| ipdC (RBS | gaaggagatatacatATGCGTACACCCTACTGTGTCGCCGATTATCTTT |
| underlined) | TAGATCGTCTGACGGACTGCGGGGCCGATCACCTGTTTGGCGT |
| SEQ ID NO: 260 | ACCGGGCGATTACAACTTGCAGTTTCTGGACCACGTCATTGAC |
| | TCACCAGATATCTGCTGGGTAGGGTGTGCGAACGAGCTTAACG |
| | CGAGCTACGCTGCTGACGGATATGCGCGTTGTAAAGGCTTTGC |
| | TGCACTTCTTACTACCTTCGGGGTCGGTGAGTTATCGGCGATG |
| | AACGGTATCGCAGGCTCGTACGCTGAGCACGTCCCGGTATTAC |
| | ACATTGTGGGAGCTCCGGGTACCGCAGCTCAACAGCGCGGAG |
| | AACTGTTACACCACACGCTGGGCGACGGAGAATTCCGCCACTT |
| | TTACCATATGTCCGAGCCAATTACTGTAGCCCAGGCTGTACTT |
| | ACAGAGCAAAATGCCTGTTACGAGATCGACCGTGTTTTGACCA |
| | CGATGCTTCGCGAGCGCCGTCCCGGGTATTTGATGCTGCCAGC |
| | CGATGTTGCCAAAAAAGCTGCGACGCCCCCAGTGAATGCCCT |
| | GACGCATAAACAAGCTCATGCCGATTCCGCCTGTTTAAAGGCT |
| | TTTCGCGATGCAGCTGAAAATAAATTAGCCATGTCGAAACGCA |
| | CCGCCTTGTTGGCGGACTTTCTGGTCCTGCGCCATGGCCTTAA |
| | ACACGCCCTTCAGAAATGGGTCAAAGAAGTCCCGATGGCCCA |
| | CGCTACGATGCTTATGGGTAAGGGGATTTTTGATGAACGTCAA |
| | GCGGGATTTTATGGAACTTATTCCGGTTCGGCGAGTACGGGGG |
| | CGGTAAAGGAAGCGATTGAGGGAGCCGACACAGTTCTTTGCG |
| | TGGGGACACGTTTCACCGATACACTGACCGCTGGATTCACACA |
| | CCAACTTACTCCGGCACAAACGATTGAGGTGCAACCCCATGCG |
| | GCTCGCGTGGGGGATGTATGGTTTACGGGCATTCCAATGAATC |
| | AAGCCATTGAGACTCTTGTCGAGCTGTGCAAACAGCACGTCCA |
| | CGCAGGACTGATGAGTTCGAGCTCTGGGGCGATTCCTTTTCCA |
| | CAACCAGATGGTAGTTTAACTCAAGAAAACTTCTGGCGCACAT |
| | TGCAAACCTTTATCCGCCCAGGTGATATCATCTTAGCAGACCA |
| | GGGTACTTCAGCCTTTGGAGCAATTGACCTGCGCTTACCAGCA |
| | GACGTGAACTTTATTGTGCAGCCGCTGTGGGGGTCTATTGGTT |
| | ATACTTTAGCTGCGGCCTTCGGAGCGCAGACAGCGTGTCCAAA |
| | CCGTCGTGTGATCGTATTGACAGGAGATGGAGCAGCGCAGTT |
| | GACCATTCAGGAGTTAGGCTCGATGTTACGCGATAAGCAGCA |
| | CCCCATTATCCTGGTCCTGAACAATGAGGGGTATACAGTTGAA |
| | CGCGCCATTCATGGTGCGGAACAACGCTACAATGACATCGCTT |
| | TATGGAATTGGACGCACATCCCCCAAGCCTTATCGTTAGATCC |
| | CCAATCGGAATGTTGGCGTGTGTCTGAAGCAGAGCAACTGGCT |
| | GATGTTCTGGAAAAAGTTGCTCATCATGAACGCCTGTCGTTGA |
| | TCGAGGTAATGTTGCCCAAGGCCGATATCCCTCCGTTACTGGG |
| | AGCCTTGACCAAGGCTTTAGAAGCCTGCAACAACGCTTAA |
| Iad1 (RBS | gaaggagatatacatATGCCCACCTTGAACTTGGACTTACCCAACGGTA |
| underlined) | TTAAGAGCACGATTCAGGCAGACCTTTTCATCAATAATAAGTT |
| SEQ ID NO: 261 | TGTGCCGGCGCTTGATGGGAAAACGTTCGCAACTATTAATCCG |
| | TCTACGGGGAAAGAGATCGGACAGGTGGCAGAGGCTTCGGCG |
| | AAGGATGTGGATCTTGCAGTTAAGGCCGCGCGTGAGGCGTTTG |
| | AAACTACTTGGGGGGAAAACACGCCAGGTGATGCTCGTGGCC |
| | GTTTACTGATTAAGCTTGCTGAGTTGGTGGAAGCGAATATTGA |
| | TGAGTTAGCGGCAATTGAATCACTGGACAATGGGAAAGCGTT |
| | CTCTATTGCTAAGTCATTCGACGTAGCTGCTGTGGCCGCAAAC |
| | TTACGTTACTACGGCGGTTGGGCTGATAAAAACCACGGTAAA |
| | GTCATGGAGGTAGACACAAAGCGCCTGAACTATACCCGCCAC |
| | GAGCCGATCGGGGTTTGCGGACAAATCATTCCGTGGAATTTCC |
| | CGCTTTTGATGTTTGCATGGAAGCTGGGTCCCGCTTTAGCCAC |
| | AGGGAACACAATTGTGTTAAAGACTGCCGAGCAGACTCCCTT |
| | AAGTGCTATCAAGATGTGTGAATTAATCGTAGAAGCCGGCTTT |
| | CCGCCCGGAGTAGTTAATGTGATCTCGGGATTCGGACCGGTGG |
| | CGGGGGCCGCGATCTCGCAACACATGGACATCGATAAGATTG |
| | CCTTTACAGGATCGACATTGGTTGGCCGCAACATTATGAAGGC |
| | AGCTGCGTCGACTAACTTAAAAAAGGTTACACTTGAGTTAGGA |
| | GGAAAATCCCCGAATATCATTTTCAAAGATGCCGACCTTGACC |
| | AAGCTGTTCGCTGGAGCGCCTTCGGTATCATGTTTAACCACGG |
| | ACAATGCTGCTGCGCTGGATCGCGCGTATATGTGGAAGAATCC |
| | ATCTATGACGCCTTCATGGAAAAAATGACTGCGCATTGTAAGG |
| | CGCTTCAAGTTGGAGATCCTTTCAGCGCGAACACCTTCCAAGG |
| | ACCACAAGTCTCGCAGTTACAATACGACCGTATCATGGAATAC |
| | ATCGAATCAGGGAAAAAAGATGCAAATCTTGCTTTAGGCGGC |
| | GTTCGCAAAGGGAATGAGGGGTATTTCATTGAGCCAACTATTT |
| | TTACAGACGTGCCGCACGACGCGAAGATTGCCAAAGAGGAGA |
| | TCTTCGGTCCAGTGGTTGTTGTGTCGAAATTTAAGGACGAAAA |
| | AGATCTGATCCGTATCGCAAATGATTCTATTTATGGTTTAGCT |
| | GCGGCAGTCTTTTCCCGCGACATCAGCCGCGCGATCGAGACAG |
| | CACACAAACTGAAAGCAGGCACGGTCTGGGTCAACTGCTATA |
| | ATCAGCTTATTCCGCAGGTGCCATTCGGAGGGTATAAGGCTTC |
| | CGGTATCGGCCGTGAGTTGGGGGAATATGCCTTGTCTAATTAC |
| | ACAAATATCAAGGCCGTCCACGTTAACCTTTCTCAACCGGCGC |
| | CCATTTGA |
| TrpEDCBA (RBS | Ctctagaaataattttgtttaactttaagaaggagatatacat |
| and leader region | atgcaaacacaaaaaccgactctcgaactgctaacctgcgaaggcgcttatcgcgacaacccgactgc |
| underlined) | gctttttcaccagttgtgtggggatcgtccggcaacgctgctgctggaatccgcagatatcgacagcaaa |
| SEQ ID NO: 262 | gatgatttaaaaagcctgctgctggtagacagtgcgctgcgcattacagcattaagtgacactgtcacaa |
| | tccaggcgctttccggcaatggagaagccctgttgacactactggataacgccttgcctgcgggtgtgg |
| | aaaatgaacaatcaccaaactgccgcgtactgcgcttcccgcctgtcagtccactgctggatgaagacg |
| | cccgcttatgctccctttcggtttttgacgctttccgcttattacagaatctgttgaatgtaccgaaggaaga |
| | acgagaagcaatgttcttcggcggcctgttctcttatgaccttgtggcgggatttgaaaatttaccgcaact |
| | gtcagcggaaaatagctgccctgatttctgtttttatctcgctgaaacgctgatggtgattgaccatcagaa |
| | aaaaagcactcgtattcaggccagcctgtttgctccgaatgaagaagaaaaacaacgtctcactgctcg |
| | cctgaacgaactacgtcagcaactgaccgaagccgcgccgccgctgccggtggtttccgtgccgcata |
| | tgcgttgtgaatgtaaccagagcgatgaagagttcggtggtgtagtgcgtttgttgcaaaaagcgattcg |
| | cgccggagaaattttccaggtggtgccatctcgccgtttctctctgccctgcccgtcaccgctggcagcc |
| | tattacgtgctgaaaaagagtaatcccagcccgtacatgttttttatgcaggataatgatttcaccctgtttg |
| | gcgcgtcgccggaaagttcgctcaagtatgacgccaccagccgccagattgagatttacccgattgcc |
| | ggaacacgtccacgcggtcgtcgtgccgatggttcgctggacagagacctcgacagccgcatcgaac |
| | tggagatgcgtaccgatcataaagagctttctgaacatctgatgctggtggatctcgcccgtaatgacctg |
| | gcacgcatttgcacacccggcagccgctacgtcgccgatctcaccaaagttgaccgttactcttacgtga |
| | tgcacctagtctcccgcgttgttggtgagctgcgccacgatctcgacgccctgcacgcttaccgcgcct |
| | gtatgaatatggggacgttaagcggtgcaccgaaagtacgcgctatgcagttaattgccgaagcagaa |
| | ggtcgtcgacgcggcagctacggcggcgcggtaggttattttaccgcgcatggcgatctcgacacctg |
| | cattgtgatccgctcggcgctggtggaaaacggtatcgccaccgtgcaagccggtgctggcgtagtcct |
| | tgattctgttccgcagtcggaagccgacgaaactcgtaataaagcccgcgctgtactgcgcgctattgcc |
| | accgcgcatcatgcacaggagacgttctaatggctgacattctgctgctcgataatatcgactcttttacgt |
| | acaacctggcagatcagttgcgcagcaatggtcataacgtggtgatttaccgcaaccatattccggcgc |
| | agaccttaattgaacgcctggcgacgatgagcaatccggtgctgatgctttctcctggccccggtgtgcc |
| | gagcgaagccggttgtatgccggaactcctcacccgcttgcgtggcaagctgccaattattggcatttgc |
| | ctcggacatcaggcgattgtcgaagcttacgggggctatgtcggtcaggcgggcgaaattcttcacggt |
| | aaagcgtcgagcattgaacatgacggtcaggcgatgtttgccggattaacaaacccgctgccagtggc |
| | gcgttatcactcgctggttggcagtaacattccggccggtttaaccatcaacgcccattttaatggcatggt |
| | gatggcggtgcgtcacgatgcagatcgcgtttgtggattccagttccatccggaatccattcttactaccc |
| | agggcgctcgcctgctggaacaaacgctggcctgggcgcagcagaaactagagccaaccaacacgc |
| | tgcaaccgattctggaaaaactgtatcaggcacagacgcttagccaacaagaaagccaccagctgtttt |
| | cagcggtggtacgtggcgagctgaagccggaacaactggcggcggcgctggtgagcatgaaaattc |
| | gcggtgaacacccgaacgagatcgccggggcagcaaccgcgctactggaaaacgccgcgccattcc |
| | cgcgcccggattatctgtttgccgatatcgtcggtactggcggtgacggcagcaacagcatcaatatttct |
| | accgccagtgcgtttgtcgccgcggcctgcgggctgaaagtggcgaaacacggcaaccgtagcgtct |
| | ccagtaaatccggctcgtcggatctgctggcggcgttcggtattaatcttgatatgaacgccgataaatcg |
| | cgccaggcgctggatgagttaggcgtctgtttcctctttgcgccgaagtatcacaccggattccgccatg |
| | cgatgccggttcgccagcaactgaaaacccgcactctgttcaacgtgctgggaccattgattaacccgg |
| | cgcatccgccgctggcgctaattggtgtttatagtccggaactggtgctgccgattgccgaaaccttgcg |
| | cgtgctggggtatcaacgcgcggcagtggtgcacagcggcgggatggatgaagtttcattacacgcg |
| | ccgacaatcgttgccgaactacatgacggcgaaattaagagctatcaattgaccgctgaagattttggcc |
| | tgacaccctaccaccaggagcaattggcaggcggaacaccggaagaaaaccgtgacattttaacacg |
| | cttgttacaaggtaaaggcgacgccgcccatgaagcagccgtcgcggcgaatgtcgccatgttaatgc |
| | gcctgcatggccatgaagatctgcaagccaatgcgcaaaccgttcttgaggtactgcgcagtggttccg |
| | cttacgacagagtcaccgcactggcggcacgagggtaaatgatgcaaaccgttttagcgaaaatcgtc |
| | gcagacaaggcgatttgggtagaaacccgcaaagagcagcaaccgctggccagttttcagaatgagg |
| | ttcagccgagcacgcgacatttttatgatgcacttcagggcgcacgcacggcgtttattctggagtgtaaa |
| | aaagcgtcgccgtcaaaaggcgtgatccgtgatgatttcgatccggcacgcattgccgccatttataaac |
| | attacgcttcggcaatttcagtgctgactgatgagaaatattttcaggggagctttgatttcctccccatcgt |
| | cagccaaatcgccccgcagccgattttatgtaaagacttcattatcgatccttaccagatctatctggcgc |
| | gctattaccaggccgatgcctgcttattaatgctttcagtactggatgacgaacaatatcgccagcttgca |
| | gccgtcgcccacagtctggagatgggtgtgctgaccgaagtcagtaatgaagaggaactggagcgcg |
| | ccattgcattgggggcaaaggtcgttggcatcaacaaccgcgatctgcgcgatttgtcgattgatctcaa |
| | ccgtacccgcgagcttgcgccgaaactggggcacaacgtgacggtaatcagcgaatccggcatcaat |
| | acttacgctcaggtgcgcgagttaagccacttcgctaacggctttctgattggttcggcgttgatggccca |
| | tgacgatttgaacgccgccgtgcgtcgggtgttgctgggtgagaataaagtatgtggcctgacacgtgg |
| | gcaagatgctaaagcagcttatgacgcgggcgcgatttacggtgggttgatttttgttgcgacatcaccg |
| | cgttgcgtcaacgttgaacaggcgcaggaagtgatggctgcagcaccgttgcagtatgttggcgtgttc |
| | cgcaatcacgatattgccgatgtggcggacaaagctaaggtgttatcgctggcggcagtgcaactgcat |
| | ggtaatgaagatcagctgtatatcgacaatctgcgtgaggctctgccagcacacgtcgccatctggaag |
| | gctttaagtgtcggtgaaactcttcccgcgcgcgattttcagcacatcgataaatatgtattcgacaacggt |
| | cagggcgggagcggacaacgtttcgactggtcactattaaatggtcaatcgcttggcaacgttctgctg |
| | gcggggggcttaggcgcagataactgcgtggaagcggcacaaaccggctgcgccgggcttgatttta |
| | attctgctgtagagtcgcaaccgggtatcaaagacgcacgtcttttggcctcggttttccagacgctgcgc |
| | gcatattaaggaaaggaacaatgacaacattacttaacccctattttggtgagtttggcggcatgtacgtg |
| | ccacaaatcctgatgcctgctctgcgccagctggaagaagcttttgtcagcgcgcaaaaagatcctgaa |
| | tttcaggctcagttcaacgacctgctgaaaaactatgccgggcgtccaaccgcgctgaccaaatgccag |
| | aacattacagccgggacgaacaccacgctgtatctgaagcgcgaagatttgctgcacggcggcgcgc |
| | ataaaactaaccaggtgctcggtcaggctttactggcgaagcggatgggtaaaactgaaattattgccg |
| | aaaccggtgccggtcagcatggcgtggcgtcggcccttgccagcgccctgctcggcctgaaatgccg |
| | aatttatatgggtgccaaagacgttgaacgccagtcgcccaacgttttccggatgcgcttaatgggtgcg |
| | gaagtgatcccggtacatagcggttccgcgaccctgaaagatgcctgtaatgaggcgctacgcgactg |
| | gtccggcagttatgaaaccgcgcactatatgctgggtaccgcagctggcccgcatccttacccgaccat |
| | tgtgcgtgagtttcagcggatgattggcgaagaaacgaaagcgcagattctggaaagagaaggtcgcc |
| | tgccggatgccgttatcgcctgtgttggcggtggttcgaatgccatcggtatgtttgcagatttcatcaacg |
| | aaaccgacgtcggcctgattggtgtggagcctggcggccacggtatcgaaactggcgagcacggcg |
| | caccgttaaaacatggtcgcgtgggcatctatttcggtatgaaagcgccgatgatgcaaaccgaagacg |
| | ggcaaattgaagagtcttactccatttctgccgggctggatttcccgtccgtcggcccgcaacatgcgtat |
| | ctcaacagcactggacgcgctgattacgtgtctattaccgacgatgaagccctggaagcctttaaaacg |
| | ctttgcctgcatgaagggatcatcccggcgctggaatcctcccacgccctggcccatgcgctgaaaatg |
| | atgcgcgaaaatccggaaaaagagcagctactggtggttaacctttccggtcgcggcgataaagacat |
| | cttcaccgttcacgatattttgaaagcacgaggggaaatctgatggaacgctacgaatctctgtttgccca |
| | gttgaaggagcgcaaagaaggcgcattcgttcctttcgtcaccctcggtgatccgggcattgagcagtc |
| | gttgaaaattatcgatacgctaattgaagccggtgctgacgcgctggagttaggcatccccttctccgac |
| | ccactggcggatggcccgacgattcaaaacgccacactgcgtgcttttgcggcgggagtaaccccgg |
| | cgcagtgctttgagatgctggcactcattcgccagaagcacccgaccattcccatcggccttttgatgtat |
| | gccaacctggtgtttaacaaaggcattgatgagttttatgccgagtgcgagaaagtcggcgtcgattcgg |
| | tgctggttgccgatgtgcccgtggaagagtccgcgcccttccgccaggccgcgttgcgtcataatgtcg |
| | cacctatctttatttgcccgccgaatgccgacgatgatttgctgcgccagatagcctcttacggtcgtggtt |
| | acacctatttgctgtcgcgagcgggcgtgaccggcgcagaaaaccgcgccgcgttacccctcaatcat |
| | ctggttgcgaagctgaaagagtacaacgctgcgcctccattgcagggatttggtatttccgccccggatc |
| | aggtaaaagccgcgattgatgcaggagctgcgggcgcgatttctggttcggccatcgttaaaatcatcg |
| | agcaacatattaatgagccagagaaaatgctggcggcactgaaagcttttgtacaaccgatgaaagcg |
| | gcgacgcgcagtta |
| trpE | atgcaaacacaaaaaccgactctcgaactgctaacctgcgaaggcgcttatcgcgacaacccgactgc |
| SEQ ID NO: 263 | gctttttcaccagttgtgtggggatcgtccggcaacgctgctgctggaatccgcagatatcgacagcaaa |
| | gatgatttaaaaagcctgctgctggtagacagtgcgctgcgcattacagcattaagtgacactgtcacaa |
| | tccaggcgctttccggcaatggagaagccctgttgacactactggataacgccttgcctgcgggtgtgg |
| | aaaatgaacaatcaccaaactgccgcgtactgcgcttcccgcctgtcagtccactgctggatgaagacg |
| | cccgcttatgctccctttcggtttttgacgctttccgcttattacagaatctgttgaatgtaccgaaggaaga |
| | acgagaagcaatgttcttcggcggcctgttctcttatgaccttgtggcgggatttgaaaatttaccgcaact |
| | gtcagcggaaaatagctgccctgatttctgtttttatctcgctgaaacgctgatggtgattgaccatcagaa |
| | aaaaagcactcgtattcaggccagcctgtttgctccgaatgaagaagaaaaacaacgtctcactgctcg |
| | cctgaacgaactacgtcagcaactgaccgaagccgcgccgccgctgccggtggtttccgtgccgcata |
| | tgcgttgtgaatgtaaccagagcgatgaagagttcggtggtgtagtgcgtttgttgcaaaaagcgattcg |
| | cgccggagaaattttccaggtggtgccatctcgccgtttctctctgccctgcccgtcaccgctggcagcc |
| | tattacgtgctgaaaaagagtaatcccagcccgtacatgttttttatgcaggataatgatttcaccctgtttg |
| | gcgcgtcgccggaaagttcgctcaagtatgacgccaccagccgccagattgagatttacccgattgcc |
| | ggaacacgtccacgcggtcgtcgtgccgatggttcgctggacagagacctcgacagccgcatcgaac |
| | tggagatgcgtaccgatcataaagagctttctgaacatctgatgctggtggatctcgcccgtaatgacctg |
| | gcacgcatttgcacacccggcagccgctacgtcgccgatctcaccaaagttgaccgttactcttacgtga |
| | tgcacctagtctcccgcgttgttggtgagctgcgccacgatctcgacgccctgcacgcttaccgcgcct |
| | gtatgaatatggggacgttaagcggtgcaccgaaagtacgcgctatgcagttaattgccgaagcagaa |
| | ggtcgtcgacgcggcagctacggcggcgcggtaggttattttaccgcgcatggcgatctcgacacctg |
| | cattgtgatccgctcggcgctggtggaaaacggtatcgccaccgtgcaagccggtgctggcgtagtcct |
| | tgattctgttccgcagtcggaagccgacgaaactcgtaataaagcccgcgctgtactgcgcgctattgcc |
| | accgcgcatcatgcacaggagacgttcta |
| trpD | atggctgacattctgctgctcgataatatcgactcttttacgtacaacctggcagatcagttgcgcagcaat |
| SEQ ID NO: 264 | ggtcataacgtggtgatttaccgcaaccatattccggcgcagaccttaattgaacgcctggcgacgatga |
| | gcaatccggtgctgatgctttctcctggccccggtgtgccgagcgaagccggttgtatgccggaactcc |
| | tcacccgcttgcgtggcaagctgccaattattggcatttgcctcggacatcaggcgattgtcgaagcttac |
| | gggggctatgtcggtcaggcgggcgaaattcttcacggtaaagcgtcgagcattgaacatgacggtca |
| | ggcgatgtttgccggattaacaaacccgctgccagtggcgcgttatcactcgctggttggcagtaacatt |
| | ccggccggtttaaccatcaacgcccattttaatggcatggtgatggcggtgcgtcacgatgcagatcgc |
| | gtttgtggattccagttccatccggaatccattcttactacccagggcgctcgcctgctggaacaaacgct |
| | ggcctgggcgcagcagaaactagagccaaccaacacgctgcaaccgattctggaaaaactgtatcag |
| | gcacagacgcttagccaacaagaaagccaccagctgttttcagcggtggtacgtggcgagctgaagc |
| | cggaacaactggcggcggcgctggtgagcatgaaaattcgcggtgaacacccgaacgagatcgccg |
| | gggcagcaaccgcgctactggaaaacgccgcgccattcccgcgcccggattatctgtttgccgatatc |
| | gtcggtactggcggtgacggcagcaacagcatcaatatttctaccgccagtgcgtttgtcgccgcggcc |
| | tgcgggctgaaagtggcgaaacacggcaaccgtagcgtctccagtaaatccggctcgtcggatctgct |
| | ggcggcgttcggtattaatcttgatatgaacgccgataaatcgcgccaggcgctggatgagttaggcgt |
| | ctgtttcctctttgcgccgaagtatcacaccggattccgccatgcgatgccggttcgccagcaactgaaa |
| | acccgcactctgttcaacgtgctgggaccattgattaacccggcgcatccgccgctggcgctaattggt |
| | gtttatagtccggaactggtgctgccgattgccgaaaccttgcgcgtgctggggtatcaacgcgcggca |
| | gtggtgcacagcggcgggatggatgaagtttcattacacgcgccgacaatcgttgccgaactacatga |
| | cggcgaaattaagagctatcaattgaccgctgaagattttggcctgacaccctaccaccaggagcaatt |
| | ggcaggcggaacaccggaagaaaaccgtgacattttaacacgcttgttacaaggtaaaggcgacgcc |
| | gcccatgaagcagccgtcgcggcgaatgtcgccatgttaatgcgcctgcatggccatgaagatctgca |
| | agccaatgcgcaaaccgttcttgaggtactgcgcagtggttccgcttacgacagagtcaccgcactggc |
| | ggcacgagggtaa |
| trpC | atgcaaaccgttttagcgaaaatcgtcgcagacaaggcgatttgggtagaaacccgcaaagagcagca |
| SEQ ID NO: 265 | accgctggccagttttcagaatgaggttcagccgagcacgcgacatttttatgatgcacttcagggcgca |
| | cgcacggcgtttattctggagtgtaaaaaagcgtcgccgtcaaaaggcgtgatccgtgatgatttcgatc |
| | cggcacgcattgccgccatttataaacattacgcttcggcaatttcagtgctgactgatgagaaatattttc |
| | aggggagctttgatttcctccccatcgtcagccaaatcgccccgcagccgattttatgtaaagacttcatta |
| | tcgatccttaccagatctatctggcgcgctattaccaggccgatgcctgcttattaatgctttcagtactgga |
| | tgacgaacaatatcgccagcttgcagccgtcgcccacagtctggagatgggtgtgctgaccgaagtca |
| | gtaatgaagaggaactggagcgcgccattgcattgggggcaaaggtcgttggcatcaacaaccgcga |
| | tctgcgcgatttgtcgattgatctcaaccgtacccgcgagcttgcgccgaaactggggcacaacgtgac |
| | ggtaatcagcgaatccggcatcaatacttacgctcaggtgcgcgagttaagccacttcgctaacggcttt |
| | ctgattggttcggcgttgatggcccatgacgatttgaacgccgccgtgcgtcgggtgttgctgggtgaga |
| | ataaagtatgtggcctgacacgtgggcaagatgctaaagcagcttatgacgcgggcgcgatttacggtg |
| | ggttgatttttgttgcgacatcaccgcgttgcgtcaacgttgaacaggcgcaggaagtgatggctgcagc |
| | accgttgcagtatgttggcgtgttccgcaatcacgatattgccgatgtggcggacaaagctaaggtgttat |
| | cgctggcggcagtgcaactgcatggtaatgaagatcagctgtatatcgacaatctgcgtgaggctctgc |
| | cagcacacgtcgccatctggaaggctttaagtgtcggtgaaactcttcccgcgcgcgattttcagcacat |
| | cgataaatatgtattcgacaacggtcagggcgggagcggacaacgtttcgactggtcactattaaatggt |
| | caatcgcttggcaacgttctgctggcggggggcttaggcgcagataactgcgtggaagcggcacaaa |
| | ccggctgcgccgggcttgattttaattctgctgtagagtcgcaaccgggtatcaaagacgcacgtcttttg |
| | gcctcggttttccagacgctgcgcgcatattaa |
| trpB | atgacaacattacttaacccctattttggtgagtttggcggcatgtacgtgccacaaatcctgatgcctgct |
| SEQ ID NO: 266 | ctgcgccagctggaagaagcttttgtcagcgcgcaaaaagatcctgaatttcaggctcagttcaacgac |
| | ctgctgaaaaactatgccgggcgtccaaccgcgctgaccaaatgccagaacattacagccgggacga |
| | acaccacgctgtatctgaagcgcgaagatttgctgcacggcggcgcgcataaaactaaccaggtgctc |
| | ggtcaggctttactggcgaagcggatgggtaaaactgaaattattgccgaaaccggtgccggtcagcat |
| | ggcgtggcgtcggcccttgccagcgccctgctcggcctgaaatgccgaatttatatgggtgccaaaga |
| | cgttgaacgccagtcgcccaacgttttccggatgcgcttaatgggtgcggaagtgatcccggtacatag |
| | cggttccgcgaccctgaaagatgcctgtaatgaggcgctacgcgactggtccggcagttatgaaaccg |
| | cgcactatatgctgggtaccgcagctggcccgcatccttacccgaccattgtgcgtgagtttcagcggat |
| | gattggcgaagaaacgaaagcgcagattctggaaagagaaggtcgcctgccggatgccgttatcgcct |
| | gtgttggcggtggttcgaatgccatcggtatgtttgcagatttcatcaacgaaaccgacgtcggcctgatt |
| | ggtgtggagcctggcggccacggtatcgaaactggcgagcacggcgcaccgttaaaacatggtcgc |
| | gtgggcatctatttcggtatgaaagcgccgatgatgcaaaccgaagacgggcaaattgaagagtcttac |
| | tccatttctgccgggctggatttcccgtccgtcggcccgcaacatgcgtatctcaacagcactggacgcg |
| | ctgattacgtgtctattaccgacgatgaagccctggaagcctttaaaacgctttgcctgcatgaagggatc |
| | atcccggcgctggaatcctcccacgccctggcccatgcgctgaaaatgatgcgcgaaaatccggaaa |
| | aagagcagctactggtggttaacctttccggtcgcggcgataaagacatcttcaccgttcacgatattttg |
| | aaagcacgaggggaaatctg |
| trpA | atggaacgctacgaatctctgtttgcccagttgaaggagcgcaaagaaggcgcattcgttcctttcgtca |
| SEQ ID NO: 267 | ccctcggtgatccgggcattgagcagtcgttgaaaattatcgatacgctaattgaagccggtgctgacgc |
| | gctggagttaggcatccccttctccgacccactggcggatggcccgacgattcaaaacgccacactgc |
| | gtgcttttgcggcgggagtaaccccggcgcagtgctttgagatgctggcactcattcgccagaagcacc |
| | cgaccattcccatcggccttttgatgtatgccaacctggtgtttaacaaaggcattgatgagttttatgccg |
| | agtgcgagaaagtcggcgtcgattcggtgctggttgccgatgtgcccgtggaagagtccgcgcccttc |
| | cgccaggccgcgttgcgtcataatgtcgcacctatctttatttgcccgccgaatgccgacgatgatttgct |
| | gcgccagatagcctcttacggtcgtggttacacctatttgctgtcgcgagcgggcgtgaccggcgcaga |
| | aaaccgcgccgcgttacccctcaatcatctggttgcgaagctgaaagagtacaacgctgcgcctccatt |
| | gcagggatttggtatttccgccccggatcaggtaaaagccgcgattgatgcaggagctgcgggcgcga |
| | tttctggttcggccatcgttaaaatcatcgagcaacatattaatgagccagagaaaatgctggcggcact |
| | gaaagcttttgtacaaccgatgaaagcggcgacgcgcagttaa |
| fbrS40FTrpE- | ctctagaaataattttgtttaactttaagaaggagatatacatatgcaaacacaaaaaccgactctcgaact |
| DCBA (leader | gctaacctgcgaaggcgcttatcgcgacaacccgactgcgctttttcaccagttgtgtggggatcgtccg |
| region and RBS | gcaacgctgctgctggaattcgcagatatcgacagcaaagatgatttaaaaagcctgctgctggtagac |
| underlined) | agtgcgctgcgcattacagcattaagtgacactgtcacaatccaggcgctttccggcaatggagaagcc |
| SEQ ID NO: 268 | ctgttgacactactggataacgccttgcctgcgggtgtggaaaatgaacaatcaccaaactgccgcgta |
| | ctgcgcttcccgcctgtcagtccactgctggatgaagacgcccgcttatgctccctttcggtttttgacgct |
| | ttccgcttattacagaatctgttgaatgtaccgaaggaagaacgagaagcaatgttcttcggcggcctgtt |
| | ctcttatgaccttgtggcgggatttgaaaatttaccgcaactgtcagcggaaaatagctgccctgatttctg |
| | tttttatctcgctgaaacgctgatggtgattgaccatcagaaaaaaagcactcgtattcaggccagcctgtt |
| | tgctccgaatgaagaagaaaaacaacgtctcactgctcgcctgaacgaactacgtcagcaactgaccg |
| | aagccgcgccgccgctgccggtggtttccgtgccgcatatgcgttgtgaatgtaaccagagcgatgaa |
| | gagttcggtggtgtagtgcgtttgttgcaaaaagcgattcgcgccggagaaattttccaggtggtgccat |
| | ctcgccgtttctctctgccctgcccgtcaccgctggcagcctattacgtgctgaaaaagagtaatcccag |
| | cccgtacatgttttttatgcaggataatgatttcaccctgtttggcgcgtcgccggaaagttcgctcaagtat |
| | gacgccaccagccgccagattgagatttacccgattgccggaacacgtccacgcggtcgtcgtgccga |
| | tggttcgctggacagagacctcgacagccgcatcgaactggagatgcgtaccgatcataaagagctttc |
| | tgaacatctgatgctggtggatctcgcccgtaatgacctggcacgcatttgcacacccggcagccgcta |
| | cgtcgccgatctcaccaaagttgaccgttactcttacgtgatgcacctagtctcccgcgttgttggtgagct |
| | gcgccacgatctcgacgccctgcacgcttaccgcgcctgtatgaatatggggacgttaagcggtgcac |
| | cgaaagtacgcgctatgcagttaattgccgaagcagaaggtcgtcgacgcggcagctacggcggcgc |
| | ggtaggttattttaccgcgcatggcgatctcgacacctgcattgtgatccgctcggcgctggtggaaaac |
| | ggtatcgccaccgtgcaagccggtgctggcgtagtccttgattctgttccgcagtcggaagccgacgaa |
| | actcgtaataaagcccgcgctgtactgcgcgctattgccaccgcgcatcatgcacaggagacgttctaa |
| | tggctgacattctgctgctcgataatatcgactcttttacgtacaacctggcagatcagttgcgcagcaatg |
| | gtcataacgtggtgatttaccgcaaccatattccggcgcagaccttaattgaacgcctggcgacgatgag |
| | caatccggtgctgatgctttctcctggccccggtgtgccgagcgaagccggttgtatgccggaactcctc |
| | acccgcttgcgtggcaagctgccaattattggcatttgcctcggacatcaggcgattgtcgaagcttacg |
| | ggggctatgtcggtcaggcgggcgaaattcttcacggtaaagcgtcgagcattgaacatgacggtcag |
| | gcgatgtttgccggattaacaaacccgctgccagtggcgcgttatcactcgctggttggcagtaacattc |
| | cggccggtttaaccatcaacgcccattttaatggcatggtgatggcggtgcgtcacgatgcagatcgcgt |
| | ttgtggattccagttccatccggaatccattcttactacccagggcgctcgcctgctggaacaaacgctg |
| | gcctgggcgcagcagaaactagagccaaccaacacgctgcaaccgattctggaaaaactgtatcagg |
| | cacagacgcttagccaacaagaaagccaccagctgttttcagcggtggtacgtggcgagctgaagcc |
| | ggaacaactggcggcggcgctggtgagcatgaaaattcgcggtgaacacccgaacgagatcgccgg |
| | ggcagcaaccgcgctactggaaaacgccgcgccattcccgcgcccggattatctgtttgccgatatcgt |
| | cggtactggcggtgacggcagcaacagcatcaatatttctaccgccagtgcgtttgtcgccgcggcctg |
| | cgggctgaaagtggcgaaacacggcaaccgtagcgtctccagtaaatccggctcgtcggatctgctgg |
| | cggcgttcggtattaatcttgatatgaacgccgataaatcgcgccaggcgctggatgagttaggcgtctg |
| | tttcctctttgcgccgaagtatcacaccggattccgccatgcgatgccggttcgccagcaactgaaaacc |
| | cgcactctgttcaacgtgctgggaccattgattaacccggcgcatccgccgctggcgctaattggtgttta |
| | tagtccggaactggtgctgccgattgccgaaaccttgcgcgtgctggggtatcaacgcgcggcagtgg |
| | tgcacagcggcgggatggatgaagtttcattacacgcgccgacaatcgttgccgaactacatgacggc |
| | gaaattaagagctatcaattgaccgctgaagattttggcctgacaccctaccaccaggagcaattggca |
| | ggcggaacaccggaagaaaaccgtgacattttaacacgcttgttacaaggtaaaggcgacgccgccc |
| | atgaagcagccgtcgcggcgaatgtcgccatgttaatgcgcctgcatggccatgaagatctgcaagcc |
| | aatgcgcaaaccgttcttgaggtactgcgcagtggttccgcttacgacagagtcaccgcactggcggca |
| | cgagggtaaatgatgcaaaccgttttagcgaaaatcgtcgcagacaaggcgatttgggtagaaacccg |
| | caaagagcagcaaccgctggccagttttcagaatgaggttcagccgagcacgcgacatttttatgatgc |
| | acttcagggcgcacgcacggcgtttattctggagtgtaaaaaagcgtcgccgtcaaaaggcgtgatcc |
| | gtgatgatttcgatccggcacgcattgccgccatttataaacattacgcttcggcaatttcagtgctgactg |
| | atgagaaatattttcaggggagctttgatttcctccccatcgtcagccaaatcgccccgcagccgattttat |
| | gtaaagacttcattatcgatccttaccagatctatctggcgcgctattaccaggccgatgcctgcttattaat |
| | gctttcagtactggatgacgaacaatatcgccagcttgcagccgtcgcccacagtctggagatgggtgt |
| | gctgaccgaagtcagtaatgaagaggaactggagcgcgccattgcattgggggcaaaggtcgttggc |
| | atcaacaaccgcgatctgcgcgatttgtcgattgatctcaaccgtacccgcgagcttgcgccgaaactg |
| | gggcacaacgtgacggtaatcagcgaatccggcatcaatacttacgctcaggtgcgcgagttaagcca |
| | cttcgctaacggctttctgattggttcggcgttgatggcccatgacgatttgaacgccgccgtgcgtcgg |
| | gtgttgctgggtgagaataaagtatgtggcctgacacgtgggcaagatgctaaagcagcttatgacgcg |
| | ggcgcgatttacggtgggttgatttttgttgcgacatcaccgcgttgcgtcaacgttgaacaggcgcagg |
| | aagtgatggctgcagcaccgttgcagtatgttggcgtgttccgcaatcacgatattgccgatgtggcgga |
| | caaagctaaggtgttatcgctggcggcagtgcaactgcatggtaatgaagatcagctgtatatcgacaat |
| | ctgcgtgaggctctgccagcacacgtcgccatctggaaggctttaagtgtcggtgaaactcttcccgcg |
| | cgcgattttcagcacatcgataaatatgtattcgacaacggtcagggcgggagcggacaacgtttcgac |
| | tggtcactattaaatggtcaatcgcttggcaacgttctgctggcggggggcttaggcgcagataactgcg |
| | tggaagcggcacaaaccggctgcgccgggcttgattttaattctgctgtagagtcgcaaccgggtatca |
| | aagacgcacgtcttttggcctcggttttccagacgctgcgcgcatattaaggaaaggaacaatgacaac |
| | attacttaacccctattttggtgagtttggcggcatgtacgtgccacaaatcctgatgcctgctctgcgcca |
| | gctggaagaagcttttgtcagcgcgcaaaaagatcctgaatttcaggctcagttcaacgacctgctgaaa |
| | aactatgccgggcgtccaaccgcgctgaccaaatgccagaacattacagccgggacgaacaccacgc |
| | tgtatctgaagcgcgaagatttgctgcacggcggcgcgcataaaactaaccaggtgctcggtcaggctt |
| | tactggcgaagcggatgggtaaaactgaaattattgccgaaaccggtgccggtcagcatggcgtggcg |
| | tcggcccttgccagcgccctgctcggcctgaaatgccgaatttatatgggtgccaaagacgttgaacgc |
| | cagtcgcccaacgttttccggatgcgcttaatgggtgcggaagtgatcccggtacatagcggttccgcg |
| | accctgaaagatgcctgtaatgaggcgctacgcgactggtccggcagttatgaaaccgcgcactatatg |
| | ctgggtaccgcagctggcccgcatccttacccgaccattgtgcgtgagtttcagcggatgattggcgaa |
| | gaaacgaaagcgcagattctggaaagagaaggtcgcctgccggatgccgttatcgcctgtgttggcgg |
| | tggttcgaatgccatcggtatgtttgcagatttcatcaacgaaaccgacgtcggcctgattggtgtggagc |
| | ctggcggccacggtatcgaaactggcgagcacggcgcaccgttaaaacatggtcgcgtgggcatctat |
| | ttcggtatgaaagcgccgatgatgcaaaccgaagacgggcaaattgaagagtcttactccatttctgccg |
| | ggctggatttcccgtccgtcggcccgcaacatgcgtatctcaacagcactggacgcgctgattacgtgt |
| | ctattaccgacgatgaagccctggaagcctttaaaacgctttgcctgcatgaagggatcatcccggcgct |
| | ggaatcctcccacgccctggcccatgcgctgaaaatgatgcgcgaaaatccggaaaaagagcagcta |
| | ctggtggttaacctttccggtcgcggcgataaagacatcttcaccgttcacgatattttgaaagcacgagg |
| | ggaaatctgatggaacgctacgaatctctgtttgcccagttgaaggagcgcaaagaaggcgcattcgtt |
| | cctttcgtcaccctcggtgatccgggcattgagcagtcgttgaaaattatcgatacgctaattgaagccgg |
| | tgctgacgcgctggagttaggcatccccttctccgacccactggcggatggcccgacgattcaaaacg |
| | ccacactgcgtgcttttgcggcgggagtaaccccggcgcagtgctttgagatgctggcactcattcgcc |
| | agaagcacccgaccattcccatcggccttttgatgtatgccaacctggtgtttaacaaaggcattgatgag |
| | ttttatgccgagtgcgagaaagtcggcgtcgattcggtgctggttgccgatgtgcccgtggaagagtcc |
| | gcgcccttccgccaggccgcgttgcgtcataatgtcgcacctatctttatttgcccgccgaatgccgacg |
| | atgatttgctgcgccagatagcctcttacggtcgtggttacacctatttgctgtcgcgagcgggcgtgacc |
| | ggcgcagaaaaccgcgccgcgttacccctcaatcatctggttgcgaagctgaaagagtacaacgctgc |
| | gcctccattgcagggatttggtatttccgccccggatcaggtaaaagccgcgattgatgcaggagctgc |
| | gggcgcgatttctggttcggccatcgttaaaatcatcgagcaacatattaatgagccagagaaaatgctg |
| | gcggcactgaaagcttttgtacaaccgatgaaagcggcgacgcgcagttaa |
| fbrTrpE | atgcaaacacaaaaaccgactctcgaactgctaacctgcgaaggcgcttatcgcgacaacccgactgc |
| SEQ ID NO: 269 | gctttttcaccagttgtgtggggatcgtccggcaacgctgctgctggaattcgcagatatcgacagcaaa |
| | gatgatttaaaaagcctgctgctggtagacagtgcgctgcgcattacagcattaagtgacactgtcacaa |
| | tccaggcgctttccggcaatggagaagccctgttgacactactggataacgccttgcctgcgggtgtgg |
| | aaaatgaacaatcaccaaactgccgcgtactgcgcttcccgcctgtcagtccactgctggatgaagacg |
| | cccgcttatgctccctttcggtttttgacgctttccgcttattacagaatctgttgaatgtaccgaaggaaga |
| | acgagaagcaatgttcttcggcggcctgttctcttatgaccttgtggcgggatttgaaaatttaccgcaact |
| | gtcagcggaaaatagctgccctgatttctgtttttatctcgctgaaacgctgatggtgattgaccatcagaa |
| | aaaaagcactcgtattcaggccagcctgtttgctccgaatgaagaagaaaaacaacgtctcactgctcg |
| | cctgaacgaactacgtcagcaactgaccgaagccgcgccgccgctgccggtggtttccgtgccgcata |
| | tgcgttgtgaatgtaaccagagcgatgaagagttcggtggtgtagtgcgtttgttgcaaaaagcgattcg |
| | cgccggagaaattttccaggtggtgccatctcgccgtttctctctgccctgcccgtcaccgctggcagcc |
| | tattacgtgctgaaaaagagtaatcccagcccgtacatgttttttatgcaggataatgatttcaccctgtttg |
| | gcgcgtcgccggaaagttcgctcaagtatgacgccaccagccgccagattgagatttacccgattgcc |
| | ggaacacgtccacgcggtcgtcgtgccgatggttcgctggacagagacctcgacagccgcatcgaac |
| | tggagatgcgtaccgatcataaagagctttctgaacatctgatgctggtggatctcgcccgtaatgacctg |
| | gcacgcatttgcacacccggcagccgctacgtcgccgatctcaccaaagttgaccgttactcttacgtga |
| | tgcacctagtctcccgcgttgttggtgagctgcgccacgatctcgacgccctgcacgcttaccgcgcct |
| | gtatgaatatggggacgttaagcggtgcaccgaaagtacgcgctatgcagttaattgccgaagcagaa |
| | ggtcgtcgacgcggcagctacggcggcgcggtaggttattttaccgcgcatggcgatctcgacacctg |
| | cattgtgatccgctcggcgctggtggaaaacggtatcgccaccgtgcaagccggtgctggcgtagtcct |
| | tgattctgttccgcagtcggaagccgacgaaactcgtaataaagcccgcgctgtactgcgcgctattgcc |
| | accgcgcatcatgcacaggagacgttcta |
| fbrAroG-trpDH- | ctctagaaataattttgtttaactttaagaaggagatatacatatgaattatcagaacgacgatttacgcatc |
| fldABCDacuIfldH | aaagaaatcaaagagttacttcctcctgtcgcattgctggaaaaattccccgctactgaaaatgccgcga |
| (leader region and | atacggtcgcccatgcccgaaaagcgatccataagatcctgaaaggtaatgatgatcgcctgttggtgg |
| RBS underlined) | tgattggcccatgctcaattcatgatcctgtcgcggctaaagagtatgccactcgcttgctgacgctgcgt |
| SEQ ID NO: 270 | gaagagctgcaagatgagctggaaatcgtgatgcgcgtctattttgaaaagccgcgtactacggtgggc |
| | tggaaagggctgattaacgatccgcatatggataacagcttccagatcaacgacggtctgcgtattgccc |
| | gcaaattgctgctcgatattaacgacagcggtctgccagcggcgggtgaattcctggatatgatcaccct |
| | acaatatctcgctgacctgatgagctggggcgcaattggcgcacgtaccaccgaatcgcaggtgcacc |
| | gcgaactggcgtctggtctttcttgtccggtaggtttcaaaaatggcactgatggtacgattaaagtggct |
| | atcgatgccattaatgccgccggtgcgccgcactgcttcctgtccgtaacgaaatgggggcattcggcg |
| | attgtgaataccagcggtaacggcgattgccatatcattctgcgcggcggtaaagagcctaactacagc |
| | gcgaagcacgttgctgaagtgaaagaagggctgaacaaagcaggcctgccagcgcaggtgatgatc |
| | gatttcagccatgctaactcgtcaaaacaattcaaaaagcagatggatgtttgtactgacgtttgccagca |
| | gattgccggtggcgaaaaggccattattggcgtgatggtggaaagccatctggtggaaggcaatcaga |
| | gcctcgagagcggggaaccgctggcctacggtaagagcatcaccgatgcctgcattggctgggatga |
| | taccgatgctctgttacgtcaactggcgagtgcagtaaaagcgcgtcgcgggtaaTACTtaagaag |
| | gagatatacatATGCTGTTATTCGAGACTGTGCGTGAAATGGGTCAT |
| | GAGCAAGTCCTTTTCTGTCATAGCAAGAATCCCGAGATCAAGG |
| | CAATTATCGCAATCCACGATACCACCTTAGGACCGGCTATGGG |
| | CGCAACTCGTATCTTACCTTATATTAATGAGGAGGCTGCCCTG |
| | AAAGATGCATTACGTCTGTCCCGCGGAATGACTTACAAAGCA |
| | GCCTGCGCCAATATTCCCGCCGGGGGCGGCAAAGCCGTCATC |
| | ATCGCTAACCCCGAAAACAAGACCGATGACCTGTTACGCGCA |
| | TACGGCCGTTTCGTGGACAGCTTGAACGGCCGTTTCATCACCG |
| | GGCAGGACGTTAACATTACGCCCGACGACGTTCGCACTATTTC |
| | GCAGGAGACTAAGTACGTGGTAGGCGTCTCAGAAAAGTCGGG |
| | AGGGCCGGCACCTATCACCTCTCTGGGAGTATTTTTAGGCATC |
| | AAAGCCGCTGTAGAGTCGCGTTGGCAGTCTAAACGCCTGGAT |
| | GGCATGAAAGTGGCGGTGCAAGGACTTGGGAACGTAGGAAAA |
| | AATCTTTGTCGCCATCTGCATGAACACGATGTACAACTTTTTGT |
| | GTCTGATGTCGATCCAATCAAGGCCGAGGAAGTAAAACGCTT |
| | ATTCGGGGCGACTGTTGTCGAACCGACTGAAATCTATTCTTTA |
| | GATGTTGATATTTTTGCACCGTGTGCACTTGGGGGTATTTTGA |
| | ATAGCCATACCATCCCGTTCTTACAAGCCTCAATCATCGCAGG |
| | AGCAGCGAATAACCAGCTGGAGAACGAGCAACTTCATTCGCA |
| | GATGCTTGCGAAAAAGGGTATTCTTTACTCACCAGACTACGTT |
| | ATCAATGCAGGAGGACTTATCAATGTTTATAACGAAATGATCG |
| | GATATGACGAGGAAAAAGCATTCAAACAAGTTCATAACATCT |
| | ACGATACGTTATTAGCGATTTTCGAAATTGCAAAAGAACAAG |
| | GTGTAACCACCAACGACGCGGCCCGTCGTTTAGCAGAGGATC |
| | GTATCAACAACTCCAAACGCTCAAAGAGTAAAGCGATTGCGG |
| | CGTGAAATGtaagaaggagatatacatATGGAAAACAACACCAATATGT |
| | TCTCTGGAGTGAAGGTGATCGAACTGGCCAACTTTATCGCTGC |
| | TCCGGCGGCAGGTCGCTTCTTTGCTGATGGGGGAGCAGAAGTA |
| | ATTAAGATCGAATCTCCAGCAGGCGACCCGCTGCGCTACACG |
| | GCCCCATCAGAAGGACGCCCGCTTTCTCAAGAGGAAAACACA |
| | ACGTATGATTTGGAAAACGCGAATAAGAAAGCAATTGTTCTG |
| | AACTTAAAATCGGAAAAAGGAAAGAAAATTCTTCACGAGATG |
| | CTTGCTGAGGCAGACATCTTGTTAACAAATTGGCGCACGAAAG |
| | CGTTAGTCAAACAGGGGTTAGATTACGAAACACTGAAAGAGA |
| | AGTATCCAAAATTGGTATTTGCACAGATTACAGGATACGGGG |
| | AGAAAGGACCCGACAAAGACCTGCCTGGTTTCGACTACACGG |
| | CGTTTTTCGCCCGCGGAGGAGTCTCCGGTACATTATATGAAAA |
| | AGGAACTGTCCCTCCTAATGTGGTACCGGGTCTGGGTGACCAC |
| | CAGGCAGGAATGTTCTTAGCTGCCGGTATGGCTGGTGCGTTGT |
| | ATAAGGCCAAAACCACCGGACAAGGCGACAAAGTCACCGTTA |
| | GTCTGATGCATAGCGCAATGTACGGCCTGGGAATCATGATTCA |
| | GGCAGCCCAGTACAAGGACCATGGGCTGGTGTACCCGATCAA |
| | CCGTAATGAAACGCCTAATCCTTTCATCGTTTCATACAAGTCC |
| | AAAGATGATTACTTTGTCCAAGTTTGCATGCCTCCCTATGATG |
| | TGTTTTATGATCGCTTTATGACGGCCTTAGGACGTGAAGACTT |
| | GGTAGGTGACGAACGCTACAATAAGATCGAGAACTTGAAGGA |
| | TGGTCGCGCAAAAGAAGTCTATTCCATCATCGAACAACAAAT |
| | GGTAACGAAGACGAAGGACGAATGGGACAAGATTTTTCGTGA |
| | TGCAGACATTCCATTCGCTATTGCCCAAACGTGGGAAGATCTT |
| | TTAGAAGACGAGCAGGCATGGGCCAACGACTACCTGTATAAA |
| | ATGAAGTATCCCACAGGCAACGAACGTGCCCTGGTACGTTTAC |
| | CTGTGTTCTTCAAAGAAGCTGGACTTCCTGAATACAACCAGTC |
| | GCCACAGATTGCTGAGAATACCGTGGAAGTGTTAAAGGAGAT |
| | GGGATATACCGAGCAAGAAATTGAGGAGCTTGAGAAAGACAA |
| | AGACATCATGGTACGTAAAGAGAAATGAAGGTtaagaaggagatatac |
| | atATGTCAGACCGCAACAAAGAAGTGAAAGAAAAGAAGGCTA |
| | AACACTATCTGCGCGAGATCACAGCTAAACACTACAAGGAAG |
| | CGTTAGAGGCTAAAGAGCGTGGGGAGAAAGTGGGTTGGTGTG |
| | CCTCTAACTTCCCCCAAGAGATTGCAACCACGTTGGGTGTAAA |
| | GGTTGTTTATCCCGAAAACCACGCCGCCGCCGTAGCGGCACGT |
| | GGCAATGGGCAAAATATGTGCGAACACGCGGAGGCTATGGGA |
| | TTCAGTAATGATGTGTGTGGATATGCACGTGTAAATTTAGCCG |
| | TAATGGACATCGGCCATAGTGAAGATCAACCTATTCCAATGCC |
| | TGATTTCGTTCTGTGCTGTAATAATATCTGCAATCAGATGATTA |
| | AATGGTATGAACACATTGCAAAAACGTTGGATATTCCTATGAT |
| | CCTTATCGATATTCCATATAATACTGAGAACACGGTGTCTCAG |
| | GACCGCATTAAGTACATCCGCGCCCAGTTCGATGACGCTATCA |
| | AGCAACTGGAAGAAATCACTGGCAAAAAGTGGGACGAGAATA |
| | AATTCGAAGAAGTGATGAAGATTTCGCAAGAATCGGCCAAGC |
| | AATGGTTACGCGCCGCGAGCTACGCGAAATACAAACCATCAC |
| | CGTTTTCGGGCTTTGACCTTTTTAATCACATGGCTGTAGCCGTT |
| | TGTGCTCGCGGCACCCAGGAAGCCGCCGATGCATTCAAAATGT |
| | TAGCAGATGAATATGAAGAGAACGTTAAGACAGGAAAGTCTA |
| | CTTATCGCGGCGAGGAGAAGCAGCGTATCTTGTTCGAGGGCAT |
| | CGCTTGTTGGCCTTATCTGCGCCACAAGTTGACGAAACTGAGT |
| | GAATATGGAATGAACGTCACAGCTACGGTGTACGCCGAAGCT |
| | TTTGGGGTTATTTACGAAAACATGGATGAACTGATGGCCGCTT |
| | ACAATAAAGTGCCTAACTCAATCTCCTTCGAGAACGCGCTGAA |
| | GATGCGTCTTAATGCCGTTACAAGCACCAATACAGAAGGGGC |
| | TGTTATCCACATTAATCGCAGTTGTAAGCTGTGGTCAGGATTC |
| | TTATACGAACTGGCCCGTCGTTTGGAAAAGGAGACGGGGATC |
| | CCTGTTGTTTCGTTCGACGGAGATCAAGCGGATCCCCGTAACT |
| | TCTCCGAGGCTCAATATGACACTCGCATCCAAGGTTTAAATGA |
| | GGTGATGGTCGCGAAAAAAGAAGCAGAGTGAGCTTtaagaaggaga |
| | tatacatATGTCGAATAGTGACAAGTTTTTTAACGACTTCAAGGAC |
| | ATTGTGGAAAACCCAAAGAAGTATATCATGAAGCATATGGAA |
| | CAAACGGGACAAAAAGCCATCGGTTGCATGCCTTTATACACCC |
| | CAGAAGAGCTTGTCTTAGCGGCGGGTATGTTTCCTGTTGGAGT |
| | ATGGGGCTCGAATACTGAGTTGTCAAAAGCCAAGACCTACTTT |
| | CCGGCTTTTATCTGTTCTATCTTGCAAACTACTTTAGAAAACGC |
| | ATTGAATGGGGAGTATGACATGCTGTCTGGTATGATGATCACA |
| | AACTATTGCGATTCGCTGAAATGTATGGGACAAAACTTCAAAC |
| | TTACAGTGGAAAATATCGAATTCATCCCGGTTACGGTTCCACA |
| | AAACCGCAAGATGGAGGCGGGTAAAGAATTTCTGAAATCCCA |
| | GTATAAAATGAATATCGAACAACTGGAAAAAATCTCAGGGAA |
| | TAAGATCACTGACGAGAGCTTGGAGAAGGCTATTGAAATTTA |
| | CGATGAGCACCGTAAAGTCATGAACGATTTCTCTATGCTTGCG |
| | TCCAAGTACCCTGGTATCATTACGCCAACGAAACGTAACTACG |
| | TGATGAAGTCAGCGTATTATATGGACAAGAAAGAACATACAG |
| | AGAAGGTACGTCAGTTGATGGATGAAATCAAGGCCATTGAGC |
| | CTAAACCATTCGAAGGAAAACGCGTGATTACCACTGGGATCA |
| | TTGCAGATTCGGAGGACCTTTTGAAAATCTTGGAGGAGAATAA |
| | CATTGCTATCGTGGGAGATGATATTGCACACGAGTCTCGCCAA |
| | TACCGCACTTTGACCCCGGAGGCCAACACACCTATGGACCGTC |
| | TTGCTGAACAATTTGCGAACCGCGAGTGTTCGACGTTGTATGA |
| | CCCTGAAAAAAAACGTGGACAGTATATTGTCGAGATGGCAAA |
| | AGAGCGTAAGGCCGACGGAATCATCTTCTTCATGACAAAATTC |
| | TGCGATCCCGAAGAATACGATTACCCTCAGATGAAAAAAGAC |
| | TTCGAAGAAGCCGGTATTCCCCACGTTCTGATTGAGACAGACA |
| | TGCAAATGAAGAACTACGAACAAGCTCGCACCGCTATTCAAG |
| | CATTTTCAGAAACCCTTTGACGCTtaagaaggagatatacatATGCGTGC |
| | TGTCTTAATCGAGAAGTCAGATGACACCCAGAGTGTTTCAGTT |
| | ACGGAGTTGGCTGAAGACCAATTACCCGAAGGTGACGTCCTT |
| | GTGGATGTCGCGTACAGCACATTGAATTACAAGGATGCTCTTG |
| | CGATTACTGGAAAAGCACCCGTTGTACGCCGTTTTCCTATGGT |
| | CCCCGGAATTGACTTTACTGGGACTGTCGCACAGAGTTCCCAT |
| | GCTGATTTCAAGCCAGGCGACCGCGTAATTCTGAACGGATGG |
| | GGAGTTGGTGAGAAACACTGGGGCGGTCTTGCAGAACGCGCA |
| | CGCGTACGTGGGGACTGGCTTGTCCCGTTGCCAGCCCCCTTAG |
| | ACTTGCGCCAGGCTGCAATGATTGGCACTGCGGGGTACACAG |
| | CTATGCTGTGCGTGCTTGCCCTTGAGCGCCATGGAGTCGTACC |
| | TGGGAACGGCGAGATTGTCGTCTCAGGCGCAGCAGGAGGGGT |
| | AGGTTCTGTAGCAACCACACTGTTAGCAGCCAAAGGCTACGA |
| | AGTGGCCGCCGTGACCGGGCGCGCAAGCGAGGCCGAATATTT |
| | ACGCGGATTAGGCGCCGCGTCGGTCATTGATCGCAATGAATTA |
| | ACGGGGAAGGTGCGTCCATTAGGGCAGGAACGCTGGGCAGGA |
| | GGAATCGATGTAGCAGGATCAACCGTACTTGCTAATATGTTGA |
| | GCATGATGAAATACCGTGGCGTGGTGGCGGCCTGTGGCCTGG |
| | CGGCTGGAATGGACTTGCCCGCGTCTGTCGCCCCTTTTATTCTG |
| | CGTGGTATGACTTTGGCAGGGGTAGATTCAGTCATGTGCCCCA |
| | AAACTGATCGTCTGGCTGCTTGGGCACGCCTGGCATCCGACCT |
| | GGACCCTGCAAAGCTGGAAGAGATGACAACTGAATTACCGTT |
| | CTCTGAGGTGATTGAAACGGCTCCGAAGTTCTTGGATGGAACA |
| | GTGCGTGGGCGTATTGTCATTCCGGTAACACCTTGATACTtaaga |
| | aggagatatacatATGAAAATCTTGGCATACTGCGTCCGCCCAGACGA |
| | GGTAGACTCCTTTAAGAAATTTAGTGAAAAGTACGGGCATAC |
| | AGTTGATCTTATTCCAGACTCTTTTGGACCTAATGTCGCTCATT |
| | TGGCGAAGGGTTACGATGGGATTTCTATTCTGGGCAACGACAC |
| | GTGTAACCGTGAGGCACTGGAGAAGATCAAGGATTGCGGGAT |
| | CAAATATCTGGCAACCCGTACAGCCGGAGTGAACAACATTGA |
| | CTTCGATGCAGCAAAGGAGTTCGGTATTAACGTGGCTAATGTT |
| | CCCGCATATTCCCCCAACTCGGTCAGCGAATTTACCATTGGAT |
| | TGGCATTAAGTCTGACGCGTAAGATTCCATTTGCCCTGAAACG |
| | CGTGGAACTGAACAATTTTGCGCTTGGCGGCCTTATTGGTGTG |
| | GAATTGCGTAACTTAACTTTAGGAGTCATCGGTACTGGTCGCA |
| | TCGGATTGAAAGTGATTGAGGGCTTCTCTGGGTTTGGAATGAA |
| | AAAAATGATCGGTTATGACATTTTTGAAAATGAAGAAGCAAA |
| | GAAGTACATCGAATACAAATCATTAGACGAAGTTTTTAAAGA |
| | GGCTGATATTATCACTCTGCATGCGCCTCTGACAGACGACAAC |
| | TATCATATGATTGGTAAAGAATCCATTGCTAAAATGAAGGATG |
| | GGGTATTTATTATCAACGCAGCGCGTGGAGCCTTAATCGATAG |
| | TGAGGCCCTGATTGAAGGGTTAAAATCGGGGAAGATT |
| fldA | ATGGAAAACAACACCAATATGTTCTCTGGAGTGAAGGTGATC |
| SEQ ID NO: 271 | GAACTGGCCAACTTTATCGCTGCTCCGGCGGCAGGTCGCTTCT |
| | TTGCTGATGGGGGAGCAGAAGTAATTAAGATCGAATCTCCAG |
| | CAGGCGACCCGCTGCGCTACACGGCCCCATCAGAAGGACGCC |
| | CGCTTTCTCAAGAGGAAAACACAACGTATGATTTGGAAAACG |
| | CGAATAAGAAAGCAATTGTTCTGAACTTAAAATCGGAAAAAG |
| | GAAAGAAAATTCTTCACGAGATGCTTGCTGAGGCAGACATCTT |
| | GTTAACAAATTGGCGCACGAAAGCGTTAGTCAAACAGGGGTT |
| | AGATTACGAAACACTGAAAGAGAAGTATCCAAAATTGGTATT |
| | TGCACAGATTACAGGATACGGGGAGAAAGGACCCGACAAAGA |
| | CCTGCCTGGTTTCGACTACACGGCGTTTTTCGCCCGCGGAGGA |
| | GTCTCCGGTACATTATATGAAAAAGGAACTGTCCCTCCTAATG |
| | TGGTACCGGGTCTGGGTGACCACCAGGCAGGAATGTTCTTAGC |
| | TGCCGGTATGGCTGGTGCGTTGTATAAGGCCAAAACCACCGG |
| | ACAAGGCGACAAAGTCACCGTTAGTCTGATGCATAGCGCAAT |
| | GTACGGCCTGGGAATCATGATTCAGGCAGCCCAGTACAAGGA |
| | CCATGGGCTGGTGTACCCGATCAACCGTAATGAAACGCCTAAT |
| | CCTTTCATCGTTTCATACAAGTCCAAAGATGATTACTTTGTCCA |
| | AGTTTGCATGCCTCCCTATGATGTGTTTTATGATCGCTTTATGA |
| | CGGCCTTAGGACGTGAAGACTTGGTAGGTGACGAACGCTACA |
| | ATAAGATCGAGAACTTGAAGGATGGTCGCGCAAAAGAAGTCT |
| | ATTCCATCATCGAACAACAAATGGTAACGAAGACGAAGGACG |
| | AATGGGACAAGATTTTTCGTGATGCAGACATTCCATTCGCTAT |
| | TGCCCAAACGTGGGAAGATCTTTTAGAAGACGAGCAGGCATG |
| | GGCCAACGACTACCTGTATAAAATGAAGTATCCCACAGGCAA |
| | CGAACGTGCCCTGGTACGTTTACCTGTGTTCTTCAAAGAAGCT |
| | GGACTTCCTGAATACAACCAGTCGCCACAGATTGCTGAGAATA |
| | CCGTGGAAGTGTTAAAGGAGATGGGATATACCGAGCAAGAAA |
| | TTGAGGAGCTTGAGAAAGACAAAGACATCATGGTACGTAAAG |
| | AGAAATGA |
| fldB | ATGTCAGACCGCAACAAAGAAGTGAAAGAAAAGAAGGCTAA |
| SEQ ID NO: 278 | ACACTATCTGCGCGAGATCACAGCTAAACACTACAAGGAAGC |
| | GTTAGAGGCTAAAGAGCGTGGGGAGAAAGTGGGTTGGTGTGC |
| | CTCTAACTTCCCCCAAGAGATTGCAACCACGTTGGGTGTAAAG |
| | GTTGTTTATCCCGAAAACCACGCCGCCGCCGTAGCGGCACGTG |
| | GCAATGGGCAAAATATGTGCGAACACGCGGAGGCTATGGGAT |
| | TCAGTAATGATGTGTGTGGATATGCACGTGTAAATTTAGCCGT |
| | AATGGACATCGGCCATAGTGAAGATCAACCTATTCCAATGCCT |
| | GATTTCGTTCTGTGCTGTAATAATATCTGCAATCAGATGATTA |
| | AATGGTATGAACACATTGCAAAAACGTTGGATATTCCTATGAT |
| | CCTTATCGATATTCCATATAATACTGAGAACACGGTGTCTCAG |
| | GACCGCATTAAGTACATCCGCGCCCAGTTCGATGACGCTATCA |
| | AGCAACTGGAAGAAATCACTGGCAAAAAGTGGGACGAGAATA |
| | AATTCGAAGAAGTGATGAAGATTTCGCAAGAATCGGCCAAGC |
| | AATGGTTACGCGCCGCGAGCTACGCGAAATACAAACCATCAC |
| | CGTTTTCGGGCTTTGACCTTTTTAATCACATGGCTGTAGCCGTT |
| | TGTGCTCGCGGCACCCAGGAAGCCGCCGATGCATTCAAAATGT |
| | TAGCAGATGAATATGAAGAGAACGTTAAGACAGGAAAGTCTA |
| | CTTATCGCGGCGAGGAGAAGCAGCGTATCTTGTTCGAGGGCAT |
| | CGCTTGTTGGCCTTATCTGCGCCACAAGTTGACGAAACTGAGT |
| | GAATATGGAATGAACGTCACAGCTACGGTGTACGCCGAAGCT |
| | TTTGGGGTTATTTACGAAAACATGGATGAACTGATGGCCGCTT |
| | ACAATAAAGTGCCTAACTCAATCTCCTTCGAGAACGCGCTGAA |
| | GATGCGTCTTAATGCCGTTACAAGCACCAATACAGAAGGGGC |
| | TGTTATCCACATTAATCGCAGTTGTAAGCTGTGGTCAGGATTC |
| | TTATACGAACTGGCCCGTCGTTTGGAAAAGGAGACGGGGATC |
| | CCTGTTGTTTCGTTCGACGGAGATCAAGCGGATCCCCGTAACT |
| | TCTCCGAGGCTCAATATGACACTCGCATCCAAGGTTTAAATGA |
| | GGTGATGGTCGCGAAAAAAGAAGCAGAGTGA |
| fldC | ATGTCGAATAGTGACAAGTTTTTTAACGACTTCAAGGACATTG |
| SEQ ID NO: 279 | TGGAAAACCCAAAGAAGTATATCATGAAGCATATGGAACAAA |
| | CGGGACAAAAAGCCATCGGTTGCATGCCTTTATACACCCCAGA |
| | AGAGCTTGTCTTAGCGGCGGGTATGTTTCCTGTTGGAGTATGG |
| | GGCTCGAATACTGAGTTGTCAAAAGCCAAGACCTACTTTCCGG |
| | CTTTTATCTGTTCTATCTTGCAAACTACTTTAGAAAACGCATTG |
| | AATGGGGAGTATGACATGCTGTCTGGTATGATGATCACAAACT |
| | ATTGCGATTCGCTGAAATGTATGGGACAAAACTTCAAACTTAC |
| | AGTGGAAAATATCGAATTCATCCCGGTTACGGTTCCACAAAAC |
| | CGCAAGATGGAGGCGGGTAAAGAATTTCTGAAATCCCAGTAT |
| | AAAATGAATATCGAACAACTGGAAAAAATCTCAGGGAATAAG |
| | ATCACTGACGAGAGCTTGGAGAAGGCTATTGAAATTTACGAT |
| | GAGCACCGTAAAGTCATGAACGATTTCTCTATGCTTGCGTCCA |
| | AGTACCCTGGTATCATTACGCCAACGAAACGTAACTACGTGAT |
| | GAAGTCAGCGTATTATATGGACAAGAAAGAACATACAGAGAA |
| | GGTACGTCAGTTGATGGATGAAATCAAGGCCATTGAGCCTAA |
| | ACCATTCGAAGGAAAACGCGTGATTACCACTGGGATCATTGC |
| | AGATTCGGAGGACCTTTTGAAAATCTTGGAGGAGAATAACATT |
| | GCTATCGTGGGAGATGATATTGCACACGAGTCTCGCCAATACC |
| | GCACTTTGACCCCGGAGGCCAACACACCTATGGACCGTCTTGC |
| | TGAACAATTTGCGAACCGCGAGTGTTCGACGTTGTATGACCCT |
| | GAAAAAAAACGTGGACAGTATATTGTCGAGATGGCAAAAGAG |
| | CGTAAGGCCGACGGAATCATCTTCTTCATGACAAAATTCTGCG |
| | ATCCCGAAGAATACGATTACCCTCAGATGAAAAAAGACTTCG |
| | AAGAAGCCGGTATTCCCCACGTTCTGATTGAGACAGACATGCA |
| | AATGAAGAACTACGAACAAGCTCGCACCGCTATTCAAGCATTT |
| | TCAGAAACCCTTTG |
| Acul | ATGCGTGCTGTCTTAATCGAGAAGTCAGATGACACCCAGAGTG |
| SEQ ID NO: 280 | TTTCAGTTACGGAGTTGGCTGAAGACCAATTACCCGAAGGTGA |
| | CGTCCTTGTGGATGTCGCGTACAGCACATTGAATTACAAGGAT |
| | GCTCTTGCGATTACTGGAAAAGCACCCGTTGTACGCCGTTTTC |
| | CTATGGTCCCCGGAATTGACTTTACTGGGACTGTCGCACAGAG |
| | TTCCCATGCTGATTTCAAGCCAGGCGACCGCGTAATTCTGAAC |
| | GGATGGGGAGTTGGTGAGAAACACTGGGGCGGTCTTGCAGAA |
| | CGCGCACGCGTACGTGGGGACTGGCTTGTCCCGTTGCCAGCCC |
| | CCTTAGACTTGCGCCAGGCTGCAATGATTGGCACTGCGGGGTA |
| | CACAGCTATGCTGTGCGTGCTTGCCCTTGAGCGCCATGGAGTC |
| | GTACCTGGGAACGGCGAGATTGTCGTCTCAGGCGCAGCAGGA |
| | GGGGTAGGTTCTGTAGCAACCACACTGTTAGCAGCCAAAGGC |
| | TACGAAGTGGCCGCCGTGACCGGGCGCGCAAGCGAGGCCGAA |
| | TATTTACGCGGATTAGGCGCCGCGTCGGTCATTGATCGCAATG |
| | AATTAACGGGGAAGGTGCGTCCATTAGGGCAGGAACGCTGGG |
| | CAGGAGGAATCGATGTAGCAGGATCAACCGTACTTGCTAATA |
| | TGTTGAGCATGATGAAATACCGTGGCGTGGTGGCGGCCTGTGG |
| | CCTGGCGGCTGGAATGGACTTGCCCGCGTCTGTCGCCCCTTTT |
| | ATTCTGCGTGGTATGACTTTGGCAGGGGTAGATTCAGTCATGT |
| | GCCCCAAAACTGATCGTCTGGCTGCTTGGGCACGCCTGGCATC |
| | CGACCTGGACCCTGCAAAGCTGGAAGAGATGACAACTGAATT |
| | ACCGTTCTCTGAGGTGATTGAAACGGCTCCGAAGTTCTTGGAT |
| | GGAACAGTGCGTGGGCGTATTGTCATTCCGGTAACACCTTGA |
| fldH1 | ATGAAAATCTTGGCATACTGCGTCCGCCCAGACGAGGTAGACT |
| SEQ ID NO: 281 | CCTTTAAGAAATTTAGTGAAAAGTACGGGCATACAGTTGATCT |
| | TATTCCAGACTCTTTTGGACCTAATGTCGCTCATTTGGCGAAG |
| | GGTTACGATGGGATTTCTATTCTGGGCAACGACACGTGTAACC |
| | GTGAGGCACTGGAGAAGATCAAGGATTGCGGGATCAAATATC |
| | TGGCAACCCGTACAGCCGGAGTGAACAACATTGACTTCGATG |
| | CAGCAAAGGAGTTCGGTATTAACGTGGCTAATGTTCCCGCATA |
| | TTCCCCCAACTCGGTCAGCGAATTTACCATTGGATTGGCATTA |
| | AGTCTGACGCGTAAGATTCCATTTGCCCTGAAACGCGTGGAAC |
| | TGAACAATTTTGCGCTTGGCGGCCTTATTGGTGTGGAATTGCG |
| | TAACTTAACTTTAGGAGTCATCGGTACTGGTCGCATCGGATTG |
| | AAAGTGATTGAGGGCTTCTCTGGGTTTGGAATGAAAAAAATG |
| | ATCGGTTATGACATTTTTGAAAATGAAGAAGCAAAGAAGTAC |
| | ATCGAATACAAATCATTAGACGAAGTTTTTAAAGAGGCTGATA |
| | TTATCACTCTGCATGCGCCTCTGACAGACGACAACTATCATAT |
| | GATTGGTAAAGAATCCATTGCTAAAATGAAGGATGGGGTATTT |
| | ATTATCAACGCAGCGCGTGGAGCCTTAATCGATAGTGAGGCCC |
| | TGATTGAAGGGTTAAAATCGGGGAAGATTGCGGGCGCGGCTC |
| | TGGATAGCTATGAGTATGAGCAAGGTGTCTTTCACAACAATAA |
| | GATGAATGAAATTATGCAGGATGATACCTTGGAACGTCTGAA |
| | ATCTTTTCCCAACGTCGTGATCACGCCGCATTTGGGTTTTTATA |
| | CTGATGAGGCGGTTTCCAATATGGTAGAGATCACACTGATGAA |
| | CCTTCAGGAATTCGAGTTGAAAGGAACCTGTAAGAACCAGCG |
| | TGTTTGTAAATGA |
| fbrAroG-TrpDH- | Ctctagaaataattttgtttaactttaagaaggagatatacat |
| fldABCDH (RBS | atgaattatcagaacgacgatttacgcatcaaagaaatcaaagagttacttcctcctgtcgcattgctgga |
| and leader region | aaaattccccgctactgaaaatgccgcgaatacggtcgcccatgcccgaaaagcgatccataagatcct |
| SEQ ID NO: 282 | gaaaggtaatgatgatcgcctgttggtggtgattggcccatgctcaattcatgatcctgtcgcggctaaag |
| | agtatgccactcgcttgctgacgctgcgtgaagagctgcaagatgagctggaaatcgtgatgcgcgtct |
| | attttgaaaagccgcgtactacggtgggctggaaagggctgattaacgatccgcatatggataacagctt |
| | ccagatcaacgacggtctgcgtattgcccgcaaattgctgctcgatattaacgacagcggtctgccagc |
| | ggcgggtgaattcctggatatgatcaccctacaatatctcgctgacctgatgagctggggcgcaattggc |
| | gcacgtaccaccgaatcgcaggtgcaccgcgaactggcgtctggtctttcttgtccggtaggtttcaaaa |
| | atggcactgatggtacgattaaagtggctatcgatgccattaatgccgccggtgcgccgcactgcttcct |
| | gtccgtaacgaaatgggggcattcggcgattgtgaataccagcggtaacggcgattgccatatcattctg |
| | cgcggcggtaaagagcctaactacagcgcgaagcacgttgctgaagtgaaagaagggctgaacaaa |
| | gcaggcctgccagcgcaggtgatgatcgatttcagccatgctaactcgtcaaaacaattcaaaaagcag |
| | atggatgtttgtactgacgtttgccagcagattgccggtggcgaaaaggccattattggcgtgatggtgg |
| | aaagccatctggtggaaggcaatcagagcctcgagagcggggaaccgctggcctacggtaagagca |
| | tcaccgatgcctgcattggctgggatgataccgatgctctgttacgtcaactggcgagtgcagtaaaagc |
| | gcgtcgcgggtaaTACTtaagaaggagatatacatATGCTGTTATTCGAGACTGT |
| | GCGTGAAATGGGTCATGAGCAAGTCCTTTTCTGTCATAGCAAG |
| | AATCCCGAGATCAAGGCAATTATCGCAATCCACGATACCACCT |
| | TAGGACCGGCTATGGGCGCAACTCGTATCTTACCTTATATTAA |
| | TGAGGAGGCTGCCCTGAAAGATGCATTACGTCTGTCCCGCGGA |
| | ATGACTTACAAAGCAGCCTGCGCCAATATTCCCGCCGGGGGC |
| | GGCAAAGCCGTCATCATCGCTAACCCCGAAAACAAGACCGAT |
| | GACCTGTTACGCGCATACGGCCGTTTCGTGGACAGCTTGAACG |
| | GCCGTTTCATCACCGGGCAGGACGTTAACATTACGCCCGACGA |
| | CGTTCGCACTATTTCGCAGGAGACTAAGTACGTGGTAGGCGTC |
| | TCAGAAAAGTCGGGAGGGCCGGCACCTATCACCTCTCTGGGA |
| | GTATTTTTAGGCATCAAAGCCGCTGTAGAGTCGCGTTGGCAGT |
| | CTAAACGCCTGGATGGCATGAAAGTGGCGGTGCAAGGACTTG |
| | GGAACGTAGGAAAAAATCTTTGTCGCCATCTGCATGAACACG |
| | ATGTACAACTTTTTGTGTCTGATGTCGATCCAATCAAGGCCGA |
| | GGAAGTAAAACGCTTATTCGGGGCGACTGTTGTCGAACCGACT |
| | GAAATCTATTCTTTAGATGTTGATATTTTTGCACCGTGTGCACT |
| | TGGGGGTATTTTGAATAGCCATACCATCCCGTTCTTACAAGCC |
| | TCAATCATCGCAGGAGCAGCGAATAACCAGCTGGAGAACGAG |
| | CAACTTCATTCGCAGATGCTTGCGAAAAAGGGTATTCTTTACT |
| | CACCAGACTACGTTATCAATGCAGGAGGACTTATCAATGTTTA |
| | TAACGAAATGATCGGATATGACGAGGAAAAAGCATTCAAACA |
| | AGTTCATAACATCTACGATACGTTATTAGCGATTTTCGAAATT |
| | GCAAAAGAACAAGGTGTAACCACCAACGACGCGGCCCGTCGT |
| | TTAGCAGAGGATCGTATCAACAACTCCAAACGCTCAAAGAGT |
| | AAAGCGATTGCGGCGTGAAATGtaagaaggagatatacatATGGAAAAC |
| | AACACCAATATGTTCTCTGGAGTGAAGGTGATCGAACTGGCCA |
| | ACTTTATCGCTGCTCCGGCGGCAGGTCGCTTCTTTGCTGATGG |
| | GGGAGCAGAAGTAATTAAGATCGAATCTCCAGCAGGCGACCC |
| | GCTGCGCTACACGGCCCCATCAGAAGGACGCCCGCTTTCTCAA |
| | GAGGAAAACACAACGTATGATTTGGAAAACGCGAATAAGAAA |
| | GCAATTGTTCTGAACTTAAAATCGGAAAAAGGAAAGAAAATT |
| | CTTCACGAGATGCTTGCTGAGGCAGACATCTTGTTAACAAATT |
| | GGCGCACGAAAGCGTTAGTCAAACAGGGGTTAGATTACGAAA |
| | CACTGAAAGAGAAGTATCCAAAATTGGTATTTGCACAGATTAC |
| | AGGATACGGGGAGAAAGGACCCGACAAAGACCTGCCTGGTTT |
| | CGACTACACGGCGTTTTTCGCCCGCGGAGGAGTCTCCGGTACA |
| | TTATATGAAAAAGGAACTGTCCCTCCTAATGTGGTACCGGGTC |
| | TGGGTGACCACCAGGCAGGAATGTTCTTAGCTGCCGGTATGGC |
| | TGGTGCGTTGTATAAGGCCAAAACCACCGGACAAGGCGACAA |
| | AGTCACCGTTAGTCTGATGCATAGCGCAATGTACGGCCTGGGA |
| | ATCATGATTCAGGCAGCCCAGTACAAGGACCATGGGCTGGTG |
| | TACCCGATCAACCGTAATGAAACGCCTAATCCTTTCATCGTTT |
| | CATACAAGTCCAAAGATGATTACTTTGTCCAAGTTTGCATGCC |
| | TCCCTATGATGTGTTTTATGATCGCTTTATGACGGCCTTAGGAC |
| | GTGAAGACTTGGTAGGTGACGAACGCTACAATAAGATCGAGA |
| | ACTTGAAGGATGGTCGCGCAAAAGAAGTCTATTCCATCATCGA |
| | ACAACAAATGGTAACGAAGACGAAGGACGAATGGGACAAGA |
| | TTTTTCGTGATGCAGACATTCCATTCGCTATTGCCCAAACGTG |
| | GGAAGATCTTTTAGAAGACGAGCAGGCATGGGCCAACGACTA |
| | CCTGTATAAAATGAAGTATCCCACAGGCAACGAACGTGCCCT |
| | GGTACGTTTACCTGTGTTCTTCAAAGAAGCTGGACTTCCTGAA |
| | TACAACCAGTCGCCACAGATTGCTGAGAATACCGTGGAAGTG |
| | TTAAAGGAGATGGGATATACCGAGCAAGAAATTGAGGAGCTT |
| | GAGAAAGACAAAGACATCATGGTACGTAAAGAGAAATGAAG |
| | GTtaagaaggagatatacatATGTCAGACCGCAACAAAGAAGTGAAAGA |
| | AAAGAAGGCTAAACACTATCTGCGCGAGATCACAGCTAAACA |
| | CTACAAGGAAGCGTTAGAGGCTAAAGAGCGTGGGGAGAAAGT |
| | GGGTTGGTGTGCCTCTAACTTCCCCCAAGAGATTGCAACCACG |
| | TTGGGTGTAAAGGTTGTTTATCCCGAAAACCACGCCGCCGCCG |
| | TAGCGGCACGTGGCAATGGGCAAAATATGTGCGAACACGCGG |
| | AGGCTATGGGATTCAGTAATGATGTGTGTGGATATGCACGTGT |
| | AAATTTAGCCGTAATGGACATCGGCCATAGTGAAGATCAACCT |
| | ATTCCAATGCCTGATTTCGTTCTGTGCTGTAATAATATCTGCAA |
| | TCAGATGATTAAATGGTATGAACACATTGCAAAAACGTTGGAT |
| | ATTCCTATGATCCTTATCGATATTCCATATAATACTGAGAACA |
| | CGGTGTCTCAGGACCGCATTAAGTACATCCGCGCCCAGTTCGA |
| | TGACGCTATCAAGCAACTGGAAGAAATCACTGGCAAAAAGTG |
| | GGACGAGAATAAATTCGAAGAAGTGATGAAGATTTCGCAAGA |
| | ATCGGCCAAGCAATGGTTACGCGCCGCGAGCTACGCGAAATA |
| | CAAACCATCACCGTTTTCGGGCTTTGACCTTTTTAATCACATGG |
| | CTGTAGCCGTTTGTGCTCGCGGCACCCAGGAAGCCGCCGATGC |
| | ATTCAAAATGTTAGCAGATGAATATGAAGAGAACGTTAAGAC |
| | AGGAAAGTCTACTTATCGCGGCGAGGAGAAGCAGCGTATCTT |
| | GTTCGAGGGCATCGCTTGTTGGCCTTATCTGCGCCACAAGTTG |
| | ACGAAACTGAGTGAATATGGAATGAACGTCACAGCTACGGTG |
| | TACGCCGAAGCTTTTGGGGTTATTTACGAAAACATGGATGAAC |
| | TGATGGCCGCTTACAATAAAGTGCCTAACTCAATCTCCTTCGA |
| | GAACGCGCTGAAGATGCGTCTTAATGCCGTTACAAGCACCAAT |
| | ACAGAAGGGGCTGTTATCCACATTAATCGCAGTTGTAAGCTGT |
| | GGTCAGGATTCTTATACGAACTGGCCCGTCGTTTGGAAAAGGA |
| | GACGGGGATCCCTGTTGTTTCGTTCGACGGAGATCAAGCGGAT |
| | CCCCGTAACTTCTCCGAGGCTCAATATGACACTCGCATCCAAG |
| | GTTTAAATGAGGTGATGGTCGCGAAAAAAGAAGCAGAGTGAG |
| | CTTtaagaaggagatatacatATGTCGAATAGTGACAAGTTTTTTAACGA |
| | CTTCAAGGACATTGTGGAAAACCCAAAGAAGTATATCATGAA |
| | GCATATGGAACAAACGGGACAAAAAGCCATCGGTTGCATGCC |
| | TTTATACACCCCAGAAGAGCTTGTCTTAGCGGCGGGTATGTTT |
| | CCTGTTGGAGTATGGGGCTCGAATACTGAGTTGTCAAAAGCCA |
| | AGACCTACTTTCCGGCTTTTATCTGTTCTATCTTGCAAACTACT |
| | TTAGAAAACGCATTGAATGGGGAGTATGACATGCTGTCTGGTA |
| | TGATGATCACAAACTATTGCGATTCGCTGAAATGTATGGGACA |
| | AAACTTCAAACTTACAGTGGAAAATATCGAATTCATCCCGGTT |
| | ACGGTTCCACAAAACCGCAAGATGGAGGCGGGTAAAGAATTT |
| | CTGAAATCCCAGTATAAAATGAATATCGAACAACTGGAAAAA |
| | ATCTCAGGGAATAAGATCACTGACGAGAGCTTGGAGAAGGCT |
| | ATTGAAATTTACGATGAGCACCGTAAAGTCATGAACGATTTCT |
| | CTATGCTTGCGTCCAAGTACCCTGGTATCATTACGCCAACGAA |
| | ACGTAACTACGTGATGAAGTCAGCGTATTATATGGACAAGAA |
| | AGAACATACAGAGAAGGTACGTCAGTTGATGGATGAAATCAA |
| | GGCCATTGAGCCTAAACCATTCGAAGGAAAACGCGTGATTAC |
| | CACTGGGATCATTGCAGATTCGGAGGACCTTTTGAAAATCTTG |
| | GAGGAGAATAACATTGCTATCGTGGGAGATGATATTGCACAC |
| | GAGTCTCGCCAATACCGCACTTTGACCCCGGAGGCCAACACAC |
| | CTATGGACCGTCTTGCTGAACAATTTGCGAACCGCGAGTGTTC |
| | GACGTTGTATGACCCTGAAAAAAAACGTGGACAGTATATTGTC |
| | GAGATGGCAAAAGAGCGTAAGGCCGACGGAATCATCTTCTTC |
| | ATGACAAAATTCTGCGATCCCGAAGAATACGATTACCCTCAGA |
| | TGAAAAAAGACTTCGAAGAAGCCGGTATTCCCCACGTTCTGAT |
| | TGAGACAGACATGCAAATGAAGAACTACGAACAAGCTCGCAC |
| | CGCTATTCAAGCATTTTCAGAAACCCTTTGACGCTtaagaaggagatat |
| | acatATGTTCTTTACGGAGCAACACGAACTTATTCGCAAACTGGC |
| | GCGTGACTTTGCCGAACAGGAAATCGAGCCTATCGCAGACGA |
| | AGTAGATAAAACCGCAGAGTTCCCAAAAGAAATCGTGAAGAA |
| | GATGGCTCAAAATGGATTTTTCGGCATTAAAATGCCTAAAGAA |
| | TACGGAGGGGCGGGTGCGGATAACCGCGCTTATGTCACTATTA |
| | TGGAGGAAATTTCACGTGCTTCCGGGGTAGCGGGTATCTACCT |
| | GAGCTCGCCGAACAGTTTGTTAGGAACTCCCTTCTTATTGGTC |
| | GGAACCGATGAGCAAAAAGAAAAGTACCTTAAGCCTATGATC |
| | CGCGGCGAGAAGACTCTGGCGTTCGCCCTGACAGAGCCTGGT |
| | GCTGGCTCTGATGCGGGTGCGTTGGCTACTACTGCCCGTGAAG |
| | AGGGCGACTATTATATCTTAAATGGCCGCAAGACGTTTATTAC |
| | AGGGGCTCCTATTAGCGACAATATTATTGTGTTCGCAAAAACC |
| | GATATGAGCAAAGGGACCAAAGGTATCACCACTTTCATTGTG |
| | GACTCAAAGCAGGAAGGGGTAAGTTTTGGTAAGCCAGAGGAC |
| | AAAATGGGAATGATTGGTTGTCCGACAAGCGACATCATCTTGG |
| | AAAACGTTAAAGTTCATAAGTCCGACATCTTGGGAGAAGTCA |
| | ATAAGGGGTTTATTACCGCGATGAAAACACTTTCCGTTGGTCG |
| | TATCGGAGTGGCGTCACAGGCGCTTGGAATTGCACAGGCCGC |
| | CGTAGATGAGGCGGTAAAGTACGCCAAGCAACGTAAACAATT |
| | CAATCGCCCAATCGCGAAATTTCAGGCCATTCAATTTAAACTT |
| | GCCAATATGGAGACTAAATTAAATGCCGCTAAACTTCTTGTTT |
| | ATAACGCAGCGTACAAAATGGATTGTGGAGAAAAAGCCGACA |
| | AGGAAGCCTCTATGGCTAAATACTTTGCTGCTGAATCAGCGAT |
| | CCAAATCGTTAACGACGCGCTGCAAATCCATGGCGGGTATGG |
| | CTATATCAAAGACTACAAGATTGAACGTTTGTACCGCGATGTG |
| | CGTGTGATCGCTATTTATGAGGGCACTTCCGAGGTCCAACAGA |
| | TGGTTATCGCGTCCAATCTGCTGAAGTAATACTtaagaaggagatatac |
| | atATGAAAATCTTGGCATACTGCGTCCGCCCAGACGAGGTAGA |
| | CTCCTTTAAGAAATTTAGTGAAAAGTACGGGCATACAGTTGAT |
| | CTTATTCCAGACTCTTTTGGACCTAATGTCGCTCATTTGGCGAA |
| | GGGTTACGATGGGATTTCTATTCTGGGCAACGACACGTGTAAC |
| | CGTGAGGCACTGGAGAAGATCAAGGATTGCGGGATCAAATAT |
| | CTGGCAACCCGTACAGCCGGAGTGAACAACATTGACTTCGAT |
| | GCAGCAAAGGAGTTCGGTATTAACGTGGCTAATGTTCCCGCAT |
| | ATTCCCCCAACTCGGTCAGCGAATTTACCATTGGATTGGCATT |
| | AAGTCTGACGCGTAAGATTCCATTTGCCCTGAAACGCGTGGAA |
| | CTGAACAATTTTGCGCTTGGCGGCCTTATTGGTGTGGAATTGC |
| | GTAACTTAACTTTAGGAGTCATCGGTACTGGTCGCATCGGATT |
| | GAAAGTGATTGAGGGCTTCTCTGGGTTTGGAATGAAAAAAAT |
| | GATCGGTTATGACATTTTTGAAAATGAAGAAGCAAAGAAGTA |
| | CATCGAATACAAATCATTAGACGAAGTTTTTAAAGAGGCTGAT |
| | ATTATCACTCTGCATGCGCCTCTGACAGACGACAACTATCATA |
| | TGATTGGTAAAGAATCCATTGCTAAAATGAAGGATGGGGTATT |
| | TATTATCAACGCAGCGCGTGGAGCCTTAATCGATAGTGAGGCC |
| | CTGATTGAAGGGTTAAAATCGGGGAAGATTGCGGGCGCGGCT |
| | CTGGATAGCTATGAGTATGAGCAAGGTGTCTTTCACAACAATA |
| | AGATGAATGAAATTATGCAGGATGATACCTTGGAACGTCTGA |
| | AATCTTTTCCCAACGTCGTGATCACGCCGCATTTGGGTTTTTAT |
| | ACTGATGAGGCGGTTTCCAATATGGTAGAGATCACACTGATGA |
| | ACCTTCAGGAATTCGAGTTGAAAGGAACCTGTAAGAACCAGC |
| | GTGTTTGTAAATGA |
| FldD | ATGTTCTTTACGGAGCAACACGAACTTATTCGCAAACTGGCGC |
| SEQ ID NO: 283 | GTGACTTTGCCGAACAGGAAATCGAGCCTATCGCAGACGAAG |
| | TAGATAAAACCGCAGAGTTCCCAAAAGAAATCGTGAAGAAGA |
| | TGGCTCAAAATGGATTTTTCGGCATTAAAATGCCTAAAGAATA |
| | CGGAGGGGCGGGTGCGGATAACCGCGCTTATGTCACTATTATG |
| | GAGGAAATTTCACGTGCTTCCGGGGTAGCGGGTATCTACCTGA |
| | GCTCGCCGAACAGTTTGTTAGGAACTCCCTTCTTATTGGTCGG |
| | AACCGATGAGCAAAAAGAAAAGTACCTTAAGCCTATGATCCG |
| | CGGCGAGAAGACTCTGGCGTTCGCCCTGACAGAGCCTGGTGCT |
| | GGCTCTGATGCGGGTGCGTTGGCTACTACTGCCCGTGAAGAGG |
| | GCGACTATTATATCTTAAATGGCCGCAAGACGTTTATTACAGG |
| | GGCTCCTATTAGCGACAATATTATTGTGTTCGCAAAAACCGAT |
| | ATGAGCAAAGGGACCAAAGGTATCACCACTTTCATTGTGGACT |
| | CAAAGCAGGAAGGGGTAAGTTTTGGTAAGCCAGAGGACAAAA |
| | TGGGAATGATTGGTTGTCCGACAAGCGACATCATCTTGGAAAA |
| | CGTTAAAGTTCATAAGTCCGACATCTTGGGAGAAGTCAATAAG |
| | GGGTTTATTACCGCGATGAAAACACTTTCCGTTGGTCGTATCG |
| | GAGTGGCGTCACAGGCGCTTGGAATTGCACAGGCCGCCGTAG |
| | ATGAGGCGGTAAAGTACGCCAAGCAACGTAAACAATTCAATC |
| | GCCCAATCGCGAAATTTCAGGCCATTCAATTTAAACTTGCCAA |
| | TATGGAGACTAAATTAAATGCCGCTAAACTTCTTGTTTATAAC |
| | GCAGCGTACAAAATGGATTGTGGAGAAAAAGCCGACAAGGAA |
| | GCCTCTATGGCTAAATACTTTGCTGCTGAATCAGCGATCCAAA |
| | TCGTTAACGACGCGCTGCAAATCCATGGCGGGTATGGCTATAT |
| | CAAAGACTACAAGATTGAACGTTTGTACCGCGATGTGCGTGTG |
| | ATCGCTATTTATGAGGGCACTTCCGAGGTCCAACAGATGGTTA |
| | TCGCGTCCAATCTGCTGAAGTAA |

### Example 68. Tryptophan Production in an Engineered Strain of E. coli Nissle

A number of tryptophan metabolites, either host-derived (such as tryptamine or kynurerine) or intestinal bacteria-derived (such as indoleacetate or indole), have been shown to downregulate inflammation in the context of IBD, via the activation of the AhR receptor. Other tryptophan metabolites, such as the bacteria-derived indolepropionate, have been shown to help restore intestinal barrier integrity, in experimental models of colitis. In this example, the E. coli strain Nissle was engineered to produce tryptophan, the precursor to all those beneficial metabolites.

First, in order to remove the negative regulation of tryptophan biosynthetic genes mediated by the transcription factor TrpR, the trpR gene was deleted form the E. coli Nissle genome. The tryptophan operon trpEDCBA was amplified by PCR from the E. coli Nissle genomic DNA and cloned in the low-copy plasmid pSC101 under the control of the tet promoter, downstream of the tetR repressor gene. This tet-trpEDCBA plasmid was then transformed into the ΔtrpR mutant to obtain the ΔtrpR, tet-trpEDCBA strain. Subsequently, a feedback resistant version of the aroG gene (aroGG^{fbr}) from E. coli Nissle, coding for the enzyme catalyzing the first committing step towards aromatic amino acid production, was synthetized and cloned into the medium copy plasmid p15A, under the control of the tet promoter, downstream of the tetR repressor. This plasmid was transformed into the ΔtrpR, tet-trpEDCBA strain to obtain the ΔtrpR, tet-trpEDCBA, tet-aroG^{fbr} strain. Finally, a feedback resistant version of the tet-trpEBCDA construct (tet-trpE^{fbr}BCDA) was generated from the tet-trpEBCDA. Both the tet-aroG^{fbr} and the tet-trpE^{fbr}BCDA constructs were transformed into the ΔtrpR mutant to obtain the ΔtrpR, tet-trpE^{fbr}DCBA, tet-aroG^{fbr} strain.

All generated strains were grown in LB overnight with the appropriate antibiotics and subcultured 1/100 in 3 mL LB with antibiotics in culture tubes. After two hours of growth at 37 C at 250 rpm, 100 ng/mL anhydrotetracycline (ATC) was added to the culture to induce expression of the constructs. Two hours after induction, the bacterial cells were pelleted by centrifugation at 4,000 rpm for 5 min and resuspended in 3 mL M9 minimal media. Cells were spun down again at 4,000 rpm for 5 min, resuspended in 3 mL M9 minimal media with 0.5% glucose and placed at 37 C at 250 rpm. 200 uL were collected at 2 h, 4 h and 16 h and tryptophan was quantified by LC-MS/MS in the bacterial supernatant. FIG. 44A shows that tryptophan is being produced and secreted by the ΔtrpR, tet-trpEDCBA, tet-aroG^{fbr} strain. The production of tryptophan is significantly enhanced by expressing the feedback resistant version of trpE.

### Example 69. Improved Tryptophan Production by Using a Non-PTS Carbon Source and by Deleting the tnaA Gene Encoding for the Tryptophanase Enzyme Converting Tryptophan into Indole

One of the precursor molecule to tryptophan in E. coli is phosphoenolpyruvate (PEP). Only 3% of available PEP is normally used to produce aromatic acids (that include tryptophan, phenylalanine and tyrosine). When E. coli is grown using glucose as a sole carbon source, 50% of PEP is used to import glucose into the cell using the phosphotransferase system (PTS). In order to increase tryptophan production, a non-PTS oxidized sugar, glucuronate, was used to test tryptophan secretion by the engineered E. coli Nissle strain ΔtrpR, tet-trpE^{fbr} DCBA, tet-aroG^{fbr}. In addition, the tnaA gene, encoding the tryptophanase enzyme, was deleted in the ΔtrpR, tet-trpE^{fbr} DCBA, tet-aroG^{fbr} strain in order to block the conversion of tryptophan into indole to obtain the ΔtrpRΔtnaA, tet-trpE^{fbr} DCBA, tet-aroG^{fbr} strain.

the ΔtrpR, tet-trpE^{fbr} DCBA, tet-aroG^{fbr} and ΔtrpRΔtnaA, tet-trpE^{fbr} DCBA, tet-aroG^{fbr} strains were grown in LB overnight with the appropriate antibiotics and subcultured 1/100 in 3 mL LB with antibiotics in culture tubes. After two hours of growth at 37 C at 250 rpm, 100 ng/mL anhydrotetracycline (ATC) was added to the culture to induce expression of the constructs. Two hours after induction, the bacterial cells were pelleted by centrifugation at 4,000 rpm for 5 min and resuspended in 3 mL M9 minimal media. Cells were spun down again at 4,000 rpm for 5 min, resuspended in 3 mL M9 minimal media with 1% glucose or 1% glucuronate and placed at 37 C at 250 rpm or at 37 C in an anaerobic chamber. 200 uL were collected at 3 h and 16 h and tryptophan was quantified by LC-MS/MS in the bacterial supernatant. FIG. 22B shows that tryptophan production is doubled in aerobic condition when the non-PTS oxidized sugar glucoronate was used. In addition, the deletion of tnaA had a positive effect on tryptophan production at the 3 h time point in both aerobic and anaerobic conditions and at the 16 h time point, only in anaerobic condition.

### Example 70. Improved Tryptophan Production by Increasing the Rate of Serine Biosynthesis in E. coli Nissle

The last step in the tryptophan biosynthesis in E. coli consumes one molecule of serine. In this example, we demonstrate that serine availability is a limiting factor for tryptophan production and describe the construction of the tryptophan producing E. coli Nissle strains ΔtrpRΔtnaA, tet-trpE^{fbr} DCBA, tet-aroG^{fbr} serA and ΔtrpRΔtnaA, tet-trpE^{fbr} DCBA, tet-aroG^{fbr} serA^{fbr} strains.

the ΔtrpRΔtnaA, tet-trpE^{fbr} DCBA, tet-aroG^{fbr} strain was grown in LB overnight with the appropriate antibiotics and subcultured 1/100 in 3 mL LB with antibiotics in culture tubes. After two hours of growth at 37 C at 250 rpm, 100 ng/mL anhydrotetracycline (ATC) was added to the culture to induce expression of the constructs. Two hours after induction, the bacterial cells were pelleted by centrifugation at 4,000 rpm for 5 min and resuspended in 3 mL M9 minimal media. Cells were spun down again at 4,000 rpm for 5 min, resuspended in 3 mL M9 minimal media with 1% glucuronate or 1% glucuronate and 10 mM serine and placed at 37 C an anaerobic chamber. 200 uL were collected at 3 h and 16 h and tryptophan was quantified by LC-MS/MS in the bacterial supernatant. FIG. 22C shows that tryptophan production is improved three fold by serine addition.

In order to increase the rate of serine biosynthesis in the ΔtrpRΔtnaA, tet-trpE^{fbr} DCBA, tet-aroG^{fbr} strain, the serA gene from E. coli Nissle encoding the enzyme catalyzing the first step in the serine biosynthetic pathway was amplified by PCR and cloned into the tet-aroG^{fbr} plasmid by Gibson assembly. The newly generated tet-aroG^{fbr}-serA construct was then transformed into a ΔtrpRΔtnaA, tet-trpE^{fbr} DCBA strain to generate the ΔtrpRΔtnaA, tet-trpE^{fbr} DCBA, tet-aroG^{fbr}-serA strain. The tet-aroG^{fbr}-serA construct was further modified to encode a feedback resistant version of serA (serA^{fbr}). The newly generated tet-aroG^{fbr}-serA^{fbr} construct was used to produce the ΔtrpRΔtnaA, tet-trpE^{fbr} DCBA, tet-aroG^{fbr}-serA^{fbr} strain, optimized to improve the rate of serine biosynthesis and maximize tryptophan production.

### Example 71. Comparison of Various Tryptophan Producing Strains

Compare the rates of tryptophan production in the different strains generated, the following constructs and strains were generated according to methods and sequences described herein, and assayed for tryptophan production in the presence of glucuronate as a carbon source under aerobic conditions. SYN2126 comprises ΔtrpRΔtnaA (ΔtrpRΔtnaA). SYN2323 comprises ΔtrpRΔtnaA and a tetracycline inducible construct for the expression of feedback resistant aroG on a plasmid (ΔtrpRΔtnaA, tet-aroGfbr). SYN2339 comprises ΔtrpRΔtnaA and a first tetracycline inducible construct for the expression of feedback resistant aroG on a first plasmid and a second tetracycline inducible construct with the genes of the trp operon with a feedback resistant form of trpE on a second plasmid (ΔtrpRΔtnaA, tet-aroGfbr, tet-trpEfbrDCBA). SYN2473 comprises ΔtrpRΔtnaA and a first tetracycline inducible construct for the expression of feedback resistant aroG and SerA on a first plasmid and a second tetracycline inducible construct with the genes of the trp operon with a feedback resistant form of trpE on a second plasmid (ΔtrpRΔtnaA, tet-aroGfbr-serA, tet-trpEfbrDCBA). SYN2476 comprises ΔtrpRΔtnaA and a tetracycline inducible construct with the genes of the trp operon with a feedback resistant form of trpE on a plasmid (ΔtrpRΔtnaA, tet-trpEfbrDCBA).

Overnight cultures were diluted 1/100 in 3 mL LB plus antibiotics and grown for 2 hours (37C, 250 rpm). Next, cells were induced with 100 ng/mL ATC for 2 hours (37 C, 250 rpm), spun down, washed with cmL M9, spun down again and resuspended in 3 mL M9+1% glucuronate. Cells were plated for CFU counting. For the assay, the cells were placed of 37 C with shaking at 250 rpm. Supernatants were collected at 1 h, 2 h, 3 h, 4 h 16 h for HPLC analysis for tryptophan. As seen in FIG. 22D, results indicate that expressing aroG is not sufficient nor necessary under these conditions to get Trp production and that expressing serA is beneficial for tryptophan production.

### Example 71. Bacterial Production of Indole Acetic Acid (IAA)

The ability of a strain comprising tryptophan production circuits and additionally Indole-3-pyruvate decarboxylase from Enterobacter cloacae (IpdC) and Indole-3-acetaldehyde dehydrogenase from Ustilago maydis (Iad1) to produce indole acetic acid (IAA) was tested. The following strains were generated according to methods described herein and tested.

SYN2126: comprises ΔtrpR and ΔtnaA (ΔtrpRΔtnaA). SYN2339 comprises circuitry for the production of tryptophan; ΔtrpR and ΔtnaA, a first tetracline inducible trpEfbrDCBA construct on a first plasmid(pSC101), and a second tetracycline inducible aroGfbr construct on a second plasmid (ΔtrpRΔtnaA, tetR-Ptet-trpEfbrDCBA (pSC101), tetR-Ptet-aroGfbr (p15A)). SYN2342 comprises the same tryptophan production circuitry as the parental strain SYN2339, and additionally comprises trpDH-ipdC-iad1 incorporated at the end of the second construct (ΔtrpRΔtnaA, tetR-Ptet-trpEfbrDCBA (pSC101), tetR-Ptet-aroGfbr-trpDH-ipdC-iad1 (p15A)).

Overnight cultures of the strains were diluted 1/100 in 3 mL LB plus antibiotics and grown for 2 hours (37C, 250 rpm). Strains were then induced with 100 ng/mL ATC for 2 hours (37 C, 250 rpm). Cells were spun down, and resuspended in 1 mL M9+1% glucuronic acid and CFUs were quantified CFUs using the cellometer. Supernatants were collected at 1 h, 2.5 h and 18 h for LCMS analysis of tryptophan and indole acetic acid as described herein.

As seen in FIG. 23A, SYN2126 produced no tryptophan, SYN2339 produces increasing tryptophan over the time points measured, and SYN2342 containing the additional IAA producing circuitry produces amounts of IAA that are comparable to the amounts of tryptophan produced in its parent SYN2339. No tryptophan is measured, indicating that all tryptophan produced in SYN2342 is efficiently converted into IAA.

### Example 80. Tryptamine Production Comparing Two Tryptophan Decarboxylases

The efficacy of two tryptophan decarboxylases (tdc), one from Catharanthus roseus (tdc_{Cr}) and a second from Clostridium sporogenes (tdc_{Cs}) in producing tryptamine from tryptophan was tested. The following strains were generated according to methods described herein and tested.

SYN2339 comprises ΔtrpR and ΔtnaA and a tetracycline inducible trpE^{fbr}DCBA construct on a plasmid and another tetracycline inducible construct expressing aroG^{fbr} on a second plasmid (ΔtrpRΔtnaA, tetR-Rₜₑₜ-trpE^{fbr}DCBA (pSC101), tetR-Pₜₑₜ-aroG^{fbr} (p15A)). SYN2339 is used as a control which can produce tryptophan but cannot convert it to tryptamine SYN2340 comprises ΔtrpR and ΔtnaA and a tetracycline inducible trpE^{fbr}DCBA construct on a plasmid and another tetracycline inducible construct expressing aroG^{fbr} tdc_{Cr} on a second plasmid (ΔtrpRΔtnaA, tetR-Pₜₑₜ-trpE^{fbr}DCBA (pSC101), tetR-Pₜₑₜ-aroG^{fbr}-tdc_{Cr} (p15A)). SYN2794 comprises ΔtrpR and ΔtnaA and a tetracycline inducible trpE^{fbr}DCBA construct on a plasmid and another tetracycline inducible construct expressing aroG^{fbr} tdc_{Cs} on a second plasmid (ΔtrpRΔtnaA, tetR-Pₜₑₜ-trpE^{fbr}DCBA (pSC101), tetR-Pₜₑₜ-aroG^{fbr}-tdc_{Cs} (p15A)).

Overnight cultures of the strains were diluted 1/100 in 3 mL LB plus antibiotics and grown for 2 hours (37C, 250 rpm). Strains were then induced with 100 ng/mL ATC for 2 hours (37C, 250 rpm). Cells were spun down, and resuspended in 1 mL M9+1% glucuronic acid and CFUs were quantified CFUs using the cellometer. Supernatants were collected at 3 h and 18 h for LCMS analysis of tryptophan and tryptamine, as described herein.

As seen in FIG. 23B, Tdc_{Cs} from Clostridium sporogenes is more efficient than Tdc_{Cr} from Catharanthus roseus in tryptamine production and converts all the tryptophan produced into tryptamine.

### Example 81. Kynurenine Quantification in Bacterial Supernatant by LC-MS/MS

(c) Sample Preparation

Kynurenine standards (250, 100, 20, 4, 0.8, 0.16, 0.032 μg/mL) were prepared in water from Kynurenine stock in 0.5N HCl. Sample (10 μL)(and standards) were mixed with 90 μL of ACN/H₂O (60:30, v/v) in a V-bottom 96-well plate. The plate was heat-sealed with a AlumASeal foil and mixed well, and centrifuged at 4000 rpm for 5 min. 104, of the solution was transferred to a round-bottom 96-well plate, and 90 uL 0.1% formic acid in water was added to the sample. The plate was heat sealed with a ClearASeal sheet and mixed well.

(d) LC-MS/MS Method

Kynurenine was measured by liquid chromatography coupled to tandem mass spectrometry (LC-MS/MS) using a Thermo TSQ Quantum Max triple quadrupole mass spectrometer. Table 94, Table 95, and Table 96 provide the summary of the LC-MS/MS method.

TABLE 94
LC-MS/MS Method

| | |
|---|---|
| Column: | Accucore aQ column, |
| | 2.6 μm (100 × 2.1 mm) |
| Mobile Phase A: | 99.9% H2O, 0.1% |
| | Formic Acid |
| Mobile Phase B: | 99.9% ACN, 0.1% |
| | Formic Acid |
| Injection volume: | 10 uL |

TABLE 95
HPLC Method

| Time | Flow Rate | | |
|---|---|---|---|
| (min) | (μL/min) | A % | B % |
| −0.5 | 350 | 100 | 0 |
| 0.5 | 350 | 100 | 0 |
| 1.0 | 350 | 10 | 90 |
| 2.5 | 350 | 10 | 90 |
| 2.51 | 350 | 100 | 10 |

TABLE 96
Tandem Mass Spectrometry

| | |
|---|---|
| Ion Source: | HESI-II |
| Polarity: | Positive |

SRM transitions:

| | |
|---|---|
| Kynurenine: | 209.1/91.2 |
| | 209.1/146.1 |

### Example 82. Kynurenine quantification in tumor tissue by LC-MS/MS

(e) Sample Preparation

Kynurenine standards (100, 20, 4, 0.8, 0.16, 0.032, 0.0064 μg/mL) were prepared in water from Kynurenine stock in 0.5N HCl. Weighed tumor tissues were homogenized with PBS in BeadBug prefilled tubes using a FastPrep homogenizer and the homogenate was transferred into a V-bottom 96-well plate and centrifuged at 4000 rpm for 10 min. Sample (40 μL)(and standards) were mixed with 60 μL of ACN containing 1 μg/mL of Adenosine-13C₅ (used as internal standard) in the final solution in a V-bottom 96-well plate. The plate was heat-sealed with a AlumASeal foil and mixed well, and centrifuged at 4000 rpm for 5 min. 104, of the solution was transferred to a round-bottom 96-well plate, and 90 uL 0.1% formic acid in water was added to the sample. The plate was heat sealed with a ClearASeal sheet and mixed well.

(f) LC-MS/MS Method

Kynurenine was measured by liquid chromatography coupled to tandem mass spectrometry (LC-MS/MS) using a Thermo TSQ Quantum Max triple quadrupole mass spectrometer. Table 97, Table 98, and Table 99 provide the summary of the LC-MS/MS method.

TABLE 97
LC-MS/MS Method

| | |
|---|---|
| Column: | Accucore aQ column, 2.6 μm (100 × 2.1 mm) |
| Mobile Phase A: | 99.9% H2O, 0.1% Formic Acid |
| Mobile Phase B: | 99.9% ACN, 0.1% Formic Acid |
| Injection volume: | 10 uL |

TABLE 98
HPLC Method:

| Time | Flow Rate | | |
|---|---|---|---|
| (min) | (μL/min) | A % | B % |
| −0.5 | 350 | 100 | 0 |
| 0.5 | 350 | 100 | 0 |
| 1.0 | 350 | 10 | 90 |
| 2.5 | 350 | 10 | 90 |
| 2.51 | 350 | 100 | 10 |

TABLE 99
Tandem Mass Spectrometry

| | |
|---|---|
| Ion Source: | HESI-II |
| Polarity: | Positive |

SRM transitions:

| | |
|---|---|
| Kynurenine: | 209.1/91.2 |
| | 209.1/146.1 |
| Adenosine-13C₅: | 273.1/136.2 |

### Example 83. Tryptophan and Anthranilic Acid Quantification in Bacterial Supernatant by LC-MS/MS

(g) Sample Preparation

Tryptophan and Anthranilic acid stock (10 mg/mL) were prepared in 0.5N HCl and aliquoted in 1.5 mL microcentrifuge tubes (100 μL). Standards (250, 100, 20, 4, 0.8, 0.16, 0.032 μg/mL) of each were prepared in water. Sample (10 μL) (and standards) were mixed with 90 μL of ACN/H₂O (60:30, v/v) containing 1 μg/mL of Tryptophan-d5 in the final solution in a V-bottom 96-well plate. The plate was heat-sealed with a AlumASeal foil, mixed well, and centrifuged at 4000 rpm for 5 min. 10 μL of the solution was transferred into a round-bottom 96-well plate and 90 uL 0.1% formic acid in water was added to the sample. The plate was heat-sealed with a ClearASeal sheet and mixed well.

(h) LC-MS/MS Method

Tryptophan and Anthranilic acid were measured by liquid chromatography coupled to tandem mass spectrometry (LC-MS/MS) using a Thermo TSQ Quantum Max triple quadrupole mass spectrometer. Table 87 Table 88, and Table 89 provide the summary of the LC-MS/MS method.

TABLE 87
LC-MS/MS Method

| | |
|---|---|
| Column: | Accucore aQ column, 2.6 μm (100 × 2.1 mm) |
| Mobile Phase A: | 99.9% H2O, 0.1% Formic Acid |
| Mobile Phase B: | 99.9% ACN, 0.1% Formic Acid |
| Injection volume: | 10 uL |

TABLE 88
HPLC Method

| Time | Flow Rate | | |
|---|---|---|---|
| (min) | (μL/min) | A % | B % |
| −0.5 | 350 | 100 | 0 |
| 0.5 | 350 | 100 | 0 |
| 1.0 | 350 | 10 | 90 |
| 2.5 | 350 | 10 | 90 |
| 2.51 | 350 | 100 | 10 |

TABLE 89
Tandem Mass Spectrometry

| | |
|---|---|
| Ion Source: | HESI-II |
| Polarity: | Positive |

SRM transitions:

| | |
|---|---|
| Tryptophan: | 205.1/118.2 |
| Anthranilic acid: | 138.1/92.2 |
| Tryptophan-d5: | 210.1/151.1 |

### Example 84. Tryptophan and Anthranilic Acid Quantification in Tumor Tissue by LC-MS/MS

(i) Sample Preparation

Tryptophan and Anthranilic acid stock (10 mg/mL) were prepared in 0.5N HCl and aliquoted in 1.5 mL microcentrifuge tubes (100 μL). Standards (100, 20, 4, 0.8, 0.16, 0.032, 0.0064 μg/mL) of each were prepared in water. Weighed tumor tissues were homogenized with PBS in BeadBug prefilled tubes using a FastPrep homogenizer. The homogenate was transferred into a V-bottom 96-well plate and centrifuged at 4000 rpm for 10 min. 40 μL of sample (and standards) was mixed with 60 μL of ACN containing 1 μg/mL of Tryptophan-d5 in the final solution in a V-bottom 96-well plate. The plate was heat-sealed with a AlumASeal foil, mixed well, and centrifuged at 4000 rpm for 10 min. 10 μL of the solution was transferred into a round-bottom 96-well plate, and 90 uL 0.1% formic acid in water was added to the sample. The plate was heat-sealed with a ClearASeal sheet and mixed well.

(j) LC-MS/MS Method

Tryptophan and Anthranilic acid were measured by liquid chromatography coupled to tandem mass spectrometry (LC-MS/MS) using a Thermo TSQ Quantum Max triple quadrupole mass spectrometer. Table 90, Table 91, and Table 92 provide the summary of the LC-MS/MS method.

TABLE 90
LC-MS/MS Method

| | |
|---|---|
| Column: | Accucore aQ column, 2.6 μm (100 × 2.1 mm) |
| Mobile Phase A: | 99.9% H2O, 0.1% Formic Acid |
| Mobile Phase B: | 99.9% ACN, 0.1% Formic Acid |
| Injection volume: | 10 uL |

TABLE 91
HPLC Method

| Time | Flow Rate | | |
|---|---|---|---|
| (min) | (μL/min) | A % | B % |
| −0.5 | 350 | 100 | 0 |
| 0.5 | 350 | 100 | 0 |
| 1.0 | 350 | 10 | 90 |
| 2.5 | 350 | 10 | 90 |
| 2.51 | 350 | 100 | 10 |

TABLE 92
Tandem Mass Spectrometry

| | |
|---|---|
| Ion Source: | HESI-II |
| Polarity: | Positive |

SRM transitions:

| | |
|---|---|
| Tryptophan: | 205.1/118.2 |
| Anthranilic acid: | 138.1/92.2 |
| Tryptophan-d5: | 210.1/151.1 |

### Example 85. Quantification of Tryptamine in Bacterial Supernatant by Liquid Chromatography-Mass Spectrometry (LC-MS)

Tryptamine acid stock (10 mg/mL) were prepared in 0.5N HCl, aliquoted in 1.5 mL microcentrifuge tubes (100 μL), and stored at −20° C. Standards (250, 100, 20, 4, 0.8, 0.16, 0.032 μg/mL) were prepared. Samples (10 μL) and standards were mixed with 90 μL of ACN/H2O (60:30, v/v) containing 1 μg/mL of tryptamine-d5 in the final solution in a V-bottom 96-well plate. The plate was heat-sealed with a AlumASeal foil, mixed well, and centrifuged at 4000 rpm for 5 min. The solution (10 μL) was transferred into a round-bottom 96-well plate 90 uL 0.1% formic acid in water was added to the sample. The plate was again heat-sealed with a ClearASeal sheet and mixed well.

(k) LC-MS/MS Method

Tryptamine was measured by liquid chromatography coupled to tandem mass spectrometry (LC-MS/MS) using a Thermo TSQ Quantum Max triple quadrupole mass spectrometer. Table 64., Table 65, and Table 66 provide the summary of the LC-MS/MS method.

TABLE 64
ii. HPLC Method

| | |
|---|---|
| Column | Accucore aQ column, 2.6 μm (100 × 2.1 mm) |
| Mobile Phase A | 99.9% H2O, 0.1% Formic Acid |
| Mobile Phase B | 99.9% ACN, 0.1% Formic Acid |
| Injection volume | 10 uL |

TABLE 65
iii. HPLC Method:

| Time | Flow Rate | | |
|---|---|---|---|
| (min) | (μL/min) | A % | B % |
| −0.5 | 350 | 100 | 0 |
| 0.5 | 350 | 100 | 0 |
| 1.0 | 350 | 10 | 90 |
| 2.5 | 350 | 10 | 90 |
| 2.51 | 350 | 100 | 10 |

TABLE 66
iv. Tandem Mass Spectrometry

| | |
|---|---|
| Ion Source | HESI-II |
| Polarity | Positive |

SRM transitions

| | |
|---|---|
| Tryptamine | 161.1/144.1 |

### Example 86. Quantification of Tryptophan, Indole-3-Acetate, Indole-3-Lactate, Indole-3-Propionate in Bacterial Supernatant by High-Pressure Liquid Chromatography (HPLC)

Samples were thawed on ice and centrifuged at 3,220×g for 5 min at 4° C. 80 μL of the supernatant was pipetted, mixed with 20 μL 0.5% formic acid in water, and analyzed by HPLC using a Shimadzu Prominence-I. HPLC conditions used for the quantification of tryptophan, indole-3-acetate, indole-3-lactate and indole-3-propionate are described in Table 67.

TABLE 67
v. HPLC Analysis
Chromatography

| Calibration standards | 250, 100, 20, 4, 0.8 μg/mL | | |
|---|---|---|---|
| Column | Luna 3 μm C18(2) 100 Å , 100 × 2 mm (catalog# 00D-4251-B0) | | |
| Column Temperature | 40° C. | | |
| Injection Volume | 10 μL | | |
| Autosampler Temperature | 10° C. | | |
| Flow Rate | 0.5 mL/min | | |
| Mobile Phases | A: water, 0.1% FA | | |
| | B: acetonitrile, 0.1% FA | | |
| Gradient | Time (min) | % A | % B |
| | 0 | 90 | 10 |
| | 0.5 | 90 | 10 |
| | 3 | 10 | 90 |
| | 5 | 10 | 90 |
| | 5.01 | 90 | 10 |

| | |
|---|---|
| 7 | (end) |

Detection: Photodiode Array Detector (PDA)

| Polarity | Positive | |
|---|---|---|
| Start Wavelength | 190 nm | |
| End Wavelength | 800 nm | |
| Spectrum resolution | 512 | |
| Slit Width | 8 nm | |
| Compound | Wavelength (nm) | Retention time (min) |
| Tryptophan | 274 | 1.3 |
| Indole-3-acetate | 274 | 3.5 |
| Indole-lactate | 274 | 3.3 |
| Indole-3-propionate | 274 | 3.7 |

Strain Activity Table

| | PAL activity | | LAAD activity | | Total Phe degradation | |
|---|---|---|---|---|---|---|
| | (umol/he/1e9 cfu) | | (umol/he/1e9 cfu) | | (umol/he/1e9 cfu) | |
| | static (micro) | +O2 | static (micro) | +O2 | static (micro) | +O2 |
| SYN- | 4.09 ± 0.53 | 4.37 ± 0.69 | 0 | 0 | 4.1 | 4.4 |
| PKU707 | | | | | | |
| SYN- | 1.83 ± 0.13 | 2.41 ± 0.24 | 7.56 ± 0.93 | 31.23 ± 4.94 | 9.9 | 33.7 |
| PKU710 | | | | | | |
| SYN- | NT | 3.67 ± 0.27 | 8.11 ± 1.22 | 47.81 ± 3.41 | NT | 51.5 |
| PKU708 | | | | | | |

1. A recombinant bacterial cell comprising a heterologous gene sequence encoding an amino acid catabolism enzyme operably linked to a first promoter that is not associated with the gene encoding the amino acid catabolism enzyme in nature.

2. (canceled)

3. (canceled)

4. The recombinant bacterial cell of claim 1, wherein the first promoter is directly or indirectly induced by environmental conditions specific to the gut of a mammal.

5. The recombinant bacterial cell of claim 4, wherein the first promoter is directly or indirectly induced by low oxygen or anaerobic conditions.

6. The recombinant bacterial cell of claim 5, wherein the first promoter is an FNR responsive promoter.

7. The recombinant bacterial cell of claim 1, wherein the first promoter is a constitutive promoter.

8. The recombinant bacterial cell of claim 1 further comprising a heterologous gene encoding an amino acid transporter.

9. The recombinant bacterial cell of claim 8 wherein the heterologous gene encoding the amino acid transporter is operably linked to a second promoter that is not associated with the amino acid transporter gene in nature.

10. (canceled)

11. (canceled)

12. The recombinant bacterial cell of claim 9, wherein the second promoter is directly or indirectly induced by environmental conditions specific to the gut of a mammal.

13. (canceled)

14. The recombinant bacterial cell of claim 12, wherein the second promoter is an FNR responsive promoter.

15. The recombinant bacterial cell of claim 9, wherein the second promoter is a constitutive promoter.

16. The recombinant bacterial cell of claim 1, wherein the recombinant bacterial cell further comprises a genetic modification that reduces export of an amino acid from the bacterial cell.

17. (canceled)

18. The recombinant bacterial cell of claim 1, wherein the heterologous gene encoding the amino acid catabolism enzyme is located on a plasmid or a chromosome in the bacterial cell.

19. (canceled)

20. The recombinant bacterial cell of claim 8, wherein the heterologous gene encoding the amino acid transporter is located on a plasmid or a chromosome in the bacterial cell.

21. (canceled)

22. The recombinant bacterial cell of claim 9, wherein the first inducible promoter and the second inducible promoter are separate copies of the same inducible promoter; or wherein the first inducible promoter and the second inducible promoter are different promoters.

23. (canceled)

24. The recombinant bacterial cell of claim 1, wherein the recombinant bacterial cell is a recombinant probiotic bacterial cell.

25. (canceled)

26. (canceled)

27. The recombinant bacterial cell of claim 24, wherein the recombinant bacterial cell is of the species Escherichia coli strain Nissle.

28. The recombinant bacterial cell of claim 1, wherein the recombinant bacterial cell is an auxotroph in a gene that is complemented when the recombinant bacterial cell is present in a mammalian gut.

29. (canceled)

30. The recombinant bacterial cell of claim 28, wherein the recombinant bacterial cell is an auxotroph in diaminopimelic acid or an enzyme in the thymine biosynthetic pathway.

31. A pharmaceutical composition comprising the recombinant bacterial cell of claim 1 and a pharmaceutically acceptable carrier.

32. A method for treating a disease associated with amino acid metabolism in a subject, the method comprising administering the pharmaceutical composition of claim 31 to the subject.


SEQUENCE LISTING (298 sequences)

SEQ ID NO: 1 - DNA, 290 bp, Artificial Sequence
  Synthetic: FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 2 - DNA, 173 bp, Artificial Sequence
  Synthetic: FNR Responsive Promoter
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 3 - DNA, 305 bp, Artificial Sequence
  Synthetic: FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 4 - DNA, 180 bp, Artificial Sequence
  Synthetic: FNR Responsive Promoter
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 5 - DNA, 199 bp, Artificial Sequence
  Synthetic: FNR Responsive Promoter
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 6 - protein, 490 aa, Ruminococcus gnavus
  misc_feature (1)..(490): Tryptophan Decarboxylase (EC 4.1.1.28) Chain A, Ruminococcus Gnavus Tryptophan
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 7 - DNA, 18 bp, Artificial Sequence
  Synthetic: consensus sequence
ttgttgayryrtcaacwa

SEQ ID NO: 8 - DNA, 15 bp, Artificial Sequence
  Synthetic: consensus sequence
  misc_feature (8)..(8): n is a, c, g, or t
ttataatnattataa

SEQ ID NO: 9 - DNA, 24 bp, Artificial Sequence
  Synthetic: UP element helps recruit RNA polymerase
ggaaaatttttttaaaaaaaaaac

SEQ ID NO: 10 - DNA, 1251 bp, Artificial Sequence
  Synthetic: Pseudomonas fluorescens kynureninase, codon optimized for expression in E. coli
atgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 11 - DNA, 1354 bp, Artificial Sequence
  Synthetic: Pseudomonas fluorescens kynureninase, codon optimized for expression in E. coli driven by a Tet inducible promoter
taattcctaatttttgttgacactctatcattgatagagttattttaccactccctatcagtgatagagaaaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 12 - DNA, 1985 bp, Artificial Sequence
  Synthetic: Pseudomonas fluorescens kynureninase, codon optimized for expression in E. coli driven by a Tet inducible promoter with RBS and tetR in reverse orientation
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaaggag

SEQ ID NO: 13 - DNA, 1398 bp, Artificial Sequence
  Synthetic: Human kynureninase codon optimized for expression in E coli
atggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 14 - DNA, 1501 bp, Artificial Sequence
  Synthetic: Human kynureninase, codon optimized for expression in E. coli driven by a Tet inducible promoter
taattcctaatttttgttgacactctatcattgatagagttattttaccactccctatcagtgatagagaaaagtgaatatcaagacacgaggaggtaagattatggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 15 - DNA, 2127 bp, Artificial Sequence
  Synthetic: Human kynureninase codon optimized for expression in E. coli driven by a Tet inducible promoter with RBS and tetR in reverse orientation
taagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaatatcaagacacgaggaggtaagattatggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaa

SEQ ID NO: 16 - DNA, 64 bp, Artificial Sequence
  Synthetic: SYN23119 promoter
ggaaaatttttttaaaaaaaaaacttgacagctagctcagtccttggtataatgctagcacgaa

SEQ ID NO: 17 - DNA, 25 bp, Artificial Sequence
  Synthetic: RBS
ttatataaaagtgggaggtgcccga

SEQ ID NO: 18 - DNA, 94 bp, Artificial Sequence
  Synthetic: SYN23119 promoter with RBS
ggaaaatttttttaaaaaaaaaacttgacagctagctcagtccttggtataatgctagcacgaagtgaattatataaaagtgggaggtgcccga

SEQ ID NO: 19 - DNA, 951 bp, Artificial Sequence
  Synthetic: Wild-type clbA
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagatttttttacctttccatactatagatacagtagccgtgctattagttctcactgcgagcttggtgtcgatattgaacaaatagagatttagacaactctatctgaatatcagtcagcattttttactccacaggaagctactacatagtttcacttcctcgttatgaaggtcaattactttttggaaatgtggacgctcaaagagcttacatcaatatcgaggtaaggcctatctttaggactggattgtattgaatttcatttaacaaataaaaactaactcaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 20 - DNA, 424 bp, Artificial Sequence
  Synthetic: clbA knockout
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 21 - DNA, 1225 bp, Artificial Sequence
  Synthetic: Pseudomonas fluorescens, codon optimized for expression in E. coli, driven by the SYN23119; Construct can be expressed from a plasmid, e.g., p15 or can be integrated into the chromosome, e.g., at the HA3/4 site
ggaaaatttttttaaaaaaaaaacttgacagctagctcagtccttggtataatgctagcacgaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggt

SEQ ID NO: 22 - DNA, 66 bp, Artificial Sequence
  Synthetic: Lpp promoter from E. coli
ataagtgccttcccatcaaaaaaatattctcaacataaaaaactttgtgtaatacttgtaacgcta

SEQ ID NO: 23 - DNA, 25 bp, Artificial Sequence
  Synthetic: RBS
ttatataaaagtgggaggtgcccga

SEQ ID NO: 24 - DNA, 96 bp, Artificial Sequence
  Synthetic: Lpp promoter from E. coli
ataagtgccttcccatcaaaaaaatattctcaacataaaaaactttgtgtaatacttgtaacgctagtgaattatataaaagtgggaggtgcccga

SEQ ID NO: 25 - DNA, 1347 bp, Artificial Sequence
  Synthetic: Pseudomonas fluorescens kynureninase driven by Lpp promoter from E. coli;  Construct can be expressed from a plasmid, e.g., p15 or can be integrated into the chromosome, e.g., at the HA3/4 site
ataagtgccttcccatcaaaaaaatattctcaacataaaaaactttgtgtaatacttgtaacgctagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaa

SEQ ID NO: 26 - DNA, 2372 bp, Clostridium sporogenes
  misc_feature (1)..(2372): fbrAroG-Tdc (tdc from Clostridium sporogenes)
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgaaattttggcgcaagtatacgcaacaggagatggatgagaaaatcacagaatcgcttgagaagacattaaattacgataacacgaaaaccatcggcatcccaggtactaagctggatgatactgtattttatgacgatcactccttcgttaagcactctccctatttacgtacgttcatccaaaaccctaatcacattggttgtcacacgtacgataaagcagacatcttgtttggcggcacgtttgacatcgaacgcgaactgattcagcttttggccatcgatgtcttaaacggaaatgatgaggaattcgatggatatgtgacacaggggggaaccgaggcgaatattcaggcaatgtgggtttatcgtaactatttcaaaaaagaacgtaaagcaaaacatgaggaaatcgcaatcatcacgagcgcggatacccattacagtgcatataaggggagcgacttgctgaacattgatattatcaaggtcccagtagacttctattcgcgtaagatccaggagaacacgttagactcgattgtcaaggaggcgaaggaaattggaaagaagtacttcattgtcatctcaaacatgggtacgactatgtttggcagtgtagacgaccctgatctttatgctaacatttttgataagtataacttagaatacaaaatccacgtcgatggagcttttgggggtttcatttatcctatcgataataaggagtgcaaaacagatttctcgaacaagaacgtctcatccatcacgcttgacggtcacaaaatgcttcaagccccctatgggactggtatcttcgtgtcacgtaagaacttgatccataacaccctgacaaaggaagcaacgtatattgaaaacctggacgttaccctgagtgggtcccgctccggatccaacgccgttgcgatctggatggttttagcctcttatggcccctacgggtggatggagaagattaacaagttgcgcaatcgcactaagtggctttgcaagcagcttaacgacatgcgcatcaaatactataaggaggatagcatgaatatcgtcacgattgaagagcaatacgtaaataaagagattgcagagaaatacttccttgtgcctgaagtacacaatcctaccaacaattggtacaagattgtagtcatggaacatgttgaacttgacatcttgaactcccttgtttatgatttacgtaaattcaacaaggagcacctgaaggcaatgtga

SEQ ID NO: 27 - DNA, 1254 bp, Clostridium sporogenes
  misc_feature (1)..(1254): Tdc (tdc from Clostridium sporogenes)
atgaaattttggcgcaagtatacgcaacaggagatggatgagaaaatcacagaatcgcttgagaagacattaaattacgataacacgaaaaccatcggcatcccaggtactaagctggatgatactgtattttatgacgatcactccttcgttaagcactctccctatttacgtacgttcatccaaaaccctaatcacattggttgtcacacgtacgataaagcagacatcttgtttggcggcacgtttgacatcgaacgcgaactgattcagcttttggccatcgatgtcttaaacggaaatgatgaggaattcgatggatatgtgacacaggggggaaccgaggcgaatattcaggcaatgtgggtttatcgtaactatttcaaaaaagaacgtaaagcaaaacatgaggaaatcgcaatcatcacgagcgcggatacccattacagtgcatataaggggagcgacttgctgaacattgatattatcaaggtcccagtagacttctattcgcgtaagatccaggagaacacgttagactcgattgtcaaggaggcgaaggaaattggaaagaagtacttcattgtcatctcaaacatgggtacgactatgtttggcagtgtagacgaccctgatctttatgctaacatttttgataagtataacttagaatacaaaatccacgtcgatggagcttttgggggtttcatttatcctatcgataataaggagtgcaaaacagatttctcgaacaagaacgtctcatccatcacgcttgacggtcacaaaatgcttcaagccccctatgggactggtatcttcgtgtcacgtaagaacttgatccataacaccctgacaaaggaagcaacgtatattgaaaacctggacgttaccctgagtgggtcccgctccggatccaacgccgttgcgatctggatggttttagcctcttatggcccctacgggtggatggagaagattaacaagttgcgcaatcgcactaagtggctttgcaagcagcttaacgacatgcgcatcaaatactataaggaggatagcatgaatatcgtcacgattgaagagcaatacgtaaataaagagattgcagagaaatacttccttgtgcctgaagtacacaatcctaccaacaattggtacaagattgtagtcatggaacatgttgaacttgacatcttgaactcccttgtttatgatttacgtaaattcaacaaggagcacctgaaggcaatgtga

SEQ ID NO: 28 - protein, 417 aa, Clostridium sporogenes
  misc_feature (1)..(417): TDC: Tryptophan decarboxylase from Clostridium sporogenes
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 29 - DNA, 60 bp, Artificial Sequence
  Synthetic: SR36
tagaactgatgcaaaaagtgctcgacgaaggcacacagatgtgtaggctggagctgcttc

SEQ ID NO: 30 - DNA, 60 bp, Artificial Sequence
  Synthetic: SR38
gtttcgtaattagatagccaccggcgctttaatgcccggacatatgaatatcctccttag

SEQ ID NO: 31 - DNA, 52 bp, Artificial Sequence
  Synthetic: SR33
caacacgtttcctgaggaaccatgaaacagtatttagaactgatgcaaaaag

SEQ ID NO: 32 - DNA, 46 bp, Artificial Sequence
  Synthetic: SR34
cgcacactggcgtcggctctggcaggatgtttcgtaattagatagc

SEQ ID NO: 33 - DNA, 1096 bp, Artificial Sequence
  Synthetic: fbrAroG
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaa

SEQ ID NO: 34 - DNA, 1053 bp, Artificial Sequence
  Synthetic: fbrAroG
atgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaa

SEQ ID NO: 35 - DNA, 2351 bp, Artificial Sequence
  Synthetic: fbr-AroG-serA
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaatcaggttgatctggatgcggcggcaaagcgcgggatcccggtatttaacgcaccgttctcaaatacgcgctctgttgcggagctggtgattggcgaactgctgctgctattgcgcggcgtgccagaagccaatgctaaagcgcatcgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaacaaactgccgctgggcaacgccactcaggtacagcatctttctgacctgctgaatatgagcgatgtggtgagtctgcatgtaccagagaatccgtccaccaaaaatatgatgggcgcgaaagagatttcgctaatgaagcccggctcgctgctgattaatgcttcgcgcggtactgtggtggatattccagcgctgtgtgacgcgctggcgagcaaacatctggcgggggcggcaatcgacgtattcccgacggaaccggcgaccaatagcgatccatttacctctccgctgtgtgaattcgacaatgtccttctgacgccacacattggcggttcgactcaggaagcgcaggagaatatcggcttggaagttgcgggtaaattgatcaagtattctgacaatggctcaacgctctctgcggtgaacttcccggaagtctcgctgccactgcacggtgggcgtcgtctgatgcacatccacgaaaaccgtccgggcgtgctaactgcgctcaacaaaatttttgccgagcagggcgtcaacatcgccgcgcaatatctacaaacttccgcccagatgggttatgtagttattgatattgaagccgacgaagacgttgccgaaaaagcgctgcaggcaatgaaagctattccgggtaccattcgcgcccgtctgctgtactaa

SEQ ID NO: 36 - DNA, 1233 bp, Artificial Sequence
  Synthetic: SerA
atggcaaaggtatcgctggagaaagacaagattaagtttctgctggtagaaggcgtgcaccaaaaggcgctggaaagccttcgtgcagctggttacaccaacatcgaatttcacaaaggcgcgctggatgatgaacaattaaaagaatccatccgcgatgcccacttcatcggcctgcgatcccgtacccatctgactgaagacgtgatcaacgccgcagaaaaactggtcgctattggctgtttctgtatcggaacaaatcaggttgatctggatgcggcggcaaagcgcgggatcccggtatttaacgcaccgttctcaaatacgcgctctgttgcggagctggtgattggcgaactgctgctgctattgcgcggcgtgccagaagccaatgctaaagcgcatcgtggcgtgtggaacaaactggcggcgggttcttttgaagcgcgcggcaaaaagctgggtatcatcggctacggtcatattggtacgcaattgggcattctggctgaatcgctgggaatgtatgtttacttttatgatattgaaaacaaactgccgctgggcaacgccactcaggtacagcatctttctgacctgctgaatatgagcgatgtggtgagtctgcatgtaccagagaatccgtccaccaaaaatatgatgggcgcgaaagagatttcgctaatgaagcccggctcgctgctgattaatgcttcgcgcggtactgtggtggatattccagcgctgtgtgacgcgctggcgagcaaacatctggcgggggcggcaatcgacgtattcccgacggaaccggcgaccaatagcgatccatttacctctccgctgtgtgaattcgacaatgtccttctgacgccacacattggcggttcgactcaggaagcgcaggagaatatcggcttggaagttgcgggtaaattgatcaagtattctgacaatggctcaacgctctctgcggtgaacttcccggaagtctcgctgccactgcacggtgggcgtcgtctgatgcacatccacgaaaaccgtccgggcgtgctaactgcgctcaacaaaatttttgccgagcagggcgtcaacatcgccgcgcaatatctacaaacttccgcccagatgggttatgtagttattgatattgaagccgacgaagacgttgccgaaaaagcgctgcaggcaatgaaagctattccgggtaccattcgcgcccgtctgctgtactaa

SEQ ID NO: 37 - DNA, 2621 bp, C. roseus
  misc_feature (1)..(2621): fbrAroG-Tdc  (tdc from C. roseus)
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgggttctattgactcgacgaatgtggccatgtctaattctcctgttggcgagtttaagccccttgaagcagaagagttccgtaaacaggcacaccgcatggtggattttattgcggattattacaagaacgtagaaacatacccggtcctttccgaggttgaacccggctatctgcgcaaacgtattcccgaaaccgcaccatacctgccggagccacttgatgatattatgaaggatattcaaaaggacattatccccggaatgacgaactggatgtccccgaacttttacgccttcttcccggccacagttagctcagcagctttcttgggggaaatgctttcaacggcccttaacagcgtaggatttacctgggtcagttccccggcagcgactgaattagagatgatcgttatggattggcttgcgcaaattttgaaacttccaaaaagctttatgttctccggaaccgggggtggtgtcatccaaaacactacgtcagagtcgatcttgtgcactattatcgcggcccgtgaacgcgccttggaaaaattgggccctgattcaattggtaagcttgtctgctatgggtccgatcaaacgcacacaatgtttccgaaaacctgtaagttagcaggaatttatccgaataatatccgccttatccctaccacggtagaaaccgactttggcatctcaccgcaggtacttcgcaagatggtcgaagacgacgtcgctgcggggtacgttcccttatttttgtgtgccaccttgggaacgacatcaactacggcaacagatcctgtagattcgctgtccgaaatcgcaaacgagtttggtatctggattcatgtcgacgccgcatatgctggatcggcttgcatctgcccagaatttcgtcactaccttgatggcatcgaacgtgtggattccttatcgctgtctccccacaaatggcttttagcatatctggattgcacgtgcttgtgggtaaaacaacctcacctgctgcttcgcgctttaacgactaatcccgaatacttgaagaataaacagagtgatttagataaggtcgtggattttaagaactggcagatcgcaacaggacgtaagttccgctctttaaaactttggttaattctgcgttcctacggggtagttaacctgcaaagtcatatccgtagtgatgtagcgatggggaagatgtttgaggaatgggtccgttccgatagccgctttgaaatcgtcgtgccacgtaatttttcgcttgtatgctttcgcttgaaaccggatgtatctagtttacatgtcgaggaggtcaacaagaagttgttggatatgcttaactccaccggtcgcgtatatatgacgcatacaattgttggcggaatctatatgttacgtttggctgtaggtagcagcttgacagaggaacatcacgtgcgccgcgtttgggacttgatccagaagcttacggacgacctgcttaaagaggcgtga

SEQ ID NO: 38 - DNA, 1503 bp, C. roseus
  misc_feature (1)..(1503): Tdc (tdc from C. roseus)
atgggttctattgactcgacgaatgtggccatgtctaattctcctgttggcgagtttaagccccttgaagcagaagagttccgtaaacaggcacaccgcatggtggattttattgcggattattacaagaacgtagaaacatacccggtcctttccgaggttgaacccggctatctgcgcaaacgtattcccgaaaccgcaccatacctgccggagccacttgatgatattatgaaggatattcaaaaggacattatccccggaatgacgaactggatgtccccgaacttttacgccttcttcccggccacagttagctcagcagctttcttgggggaaatgctttcaacggcccttaacagcgtaggatttacctgggtcagttccccggcagcgactgaattagagatgatcgttatggattggcttgcgcaaattttgaaacttccaaaaagctttatgttctccggaaccgggggtggtgtcatccaaaacactacgtcagagtcgatcttgtgcactattatcgcggcccgtgaacgcgccttggaaaaattgggccctgattcaattggtaagcttgtctgctatgggtccgatcaaacgcacacaatgtttccgaaaacctgtaagttagcaggaatttatccgaataatatccgccttatccctaccacggtagaaaccgactttggcatctcaccgcaggtacttcgcaagatggtcgaagacgacgtcgctgcggggtacgttcccttatttttgtgtgccaccttgggaacgacatcaactacggcaacagatcctgtagattcgctgtccgaaatcgcaaacgagtttggtatctggattcatgtcgacgccgcatatgctggatcggcttgcatctgcccagaatttcgtcactaccttgatggcatcgaacgtgtggattccttatcgctgtctccccacaaatggcttttagcatatctggattgcacgtgcttgtgggtaaaacaacctcacctgctgcttcgcgctttaacgactaatcccgaatacttgaagaataaacagagtgatttagataaggtcgtggattttaagaactggcagatcgcaacaggacgtaagttccgctctttaaaactttggttaattctgcgttcctacggggtagttaacctgcaaagtcatatccgtagtgatgtagcgatggggaagatgtttgaggaatgggtccgttccgatagccgctttgaaatcgtcgtgccacgtaatttttcgcttgtatgctttcgcttgaaaccggatgtatctagtttacatgtcgaggaggtcaacaagaagttgttggatatgcttaactccaccggtcgcgtatatatgacgcatacaattgttggcggaatctatatgttacgtttggctgtaggtagcagcttgacagaggaacatcacgtgcgccgcgtttgggacttgatccagaagcttacggacgacctgcttaaagaggcgtga

SEQ ID NO: 39 - DNA, 5377 bp, Artificial Sequence
  Synthetic: fbrAroG-trpDH-ipdC-iad1
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatgcgtacaccctactgtgtcgccgattatcttttagatcgtctgacggactgcggggccgatcacctgtttggcgtaccgggcgattacaacttgcagtttctggaccacgtcattgactcaccagatatctgctgggtagggtgtgcgaacgagcttaacgcgagctacgctgctgacggatatgcgcgttgtaaaggctttgctgcacttcttactaccttcggggtcggtgagttatcggcgatgaacggtatcgcaggctcgtacgctgagcacgtcccggtattacacattgtgggagctccgggtaccgcagctcaacagcgcggagaactgttacaccacacgctgggcgacggagaattccgccacttttaccatatgtccgagccaattactgtagcccaggctgtacttacagagcaaaatgcctgttacgagatcgaccgtgttttgaccacgatgcttcgcgagcgccgtcccgggtatttgatgctgccagccgatgttgccaaaaaagctgcgacgcccccagtgaatgccctgacgcataaacaagctcatgccgattccgcctgtttaaaggcttttcgcgatgcagctgaaaataaattagccatgtcgaaacgcaccgccttgttggcggactttctggtcctgcgccatggccttaaacacgcccttcagaaatgggtcaaagaagtcccgatggcccacgctacgatgcttatgggtaaggggatttttgatgaacgtcaagcgggattttatggaacttattccggttcggcgagtacgggggcggtaaaggaagcgattgagggagccgacacagttctttgcgtggggacacgtttcaccgatacactgaccgctggattcacacaccaacttactccggcacaaacgattgaggtgcaaccccatgcggctcgcgtgggggatgtatggtttacgggcattccaatgaatcaagccattgagactcttgtcgagctgtgcaaacagcacgtccacgcaggactgatgagttcgagctctggggcgattccttttccacaaccagatggtagtttaactcaagaaaacttctggcgcacattgcaaacctttatccgcccaggtgatatcatcttagcagaccagggtacttcagcctttggagcaattgacctgcgcttaccagcagacgtgaactttattgtgcagccgctgtgggggtctattggttatactttagctgcggccttcggagcgcagacagcgtgtccaaaccgtcgtgtgatcgtattgacaggagatggagcagcgcagttgaccattcaggagttaggctcgatgttacgcgataagcagcaccccattatcctggtcctgaacaatgaggggtatacagttgaacgcgccattcatggtgcggaacaacgctacaatgacatcgctttatggaattggacgcacatcccccaagccttatcgttagatccccaatcggaatgttggcgtgtgtctgaagcagagcaactggctgatgttctggaaaaagttgctcatcatgaacgcctgtcgttgatcgaggtaatgttgcccaaggccgatatccctccgttactgggagccttgaccaaggctttagaagcctgcaacaacgcttaaaggttaagaaggagatatacatatgcccaccttgaacttggacttacccaacggtattaagagcacgattcaggcagaccttttcatcaataataagtttgtgccggcgcttgatgggaaaacgttcgcaactattaatccgtctacggggaaagagatcggacaggtggcagaggcttcggcgaaggatgtggatcttgcagttaaggccgcgcgtgaggcgtttgaaactacttggggggaaaacacgccaggtgatgctcgtggccgtttactgattaagcttgctgagttggtggaagcgaatattgatgagttagcggcaattgaatcactggacaatgggaaagcgttctctattgctaagtcattcgacgtagctgctgtggccgcaaacttacgttactacggcggttgggctgataaaaaccacggtaaagtcatggaggtagacacaaagcgcctgaactatacccgccacgagccgatcggggtttgcggacaaatcattccgtggaatttcccgcttttgatgtttgcatggaagctgggtcccgctttagccacagggaacacaattgtgttaaagactgccgagcagactcccttaagtgctatcaagatgtgtgaattaatcgtagaagccggctttccgcccggagtagttaatgtgatctcgggattcggaccggtggcgggggccgcgatctcgcaacacatggacatcgataagattgcctttacaggatcgacattggttggccgcaacattatgaaggcagctgcgtcgactaacttaaaaaaggttacacttgagttaggaggaaaatccccgaatatcattttcaaagatgccgaccttgaccaagctgttcgctggagcgccttcggtatcatgtttaaccacggacaatgctgctgcgctggatcgcgcgtatatgtggaagaatccatctatgacgccttcatggaaaaaatgactgcgcattgtaaggcgcttcaagttggagatcctttcagcgcgaacaccttccaaggaccacaagtctcgcagttacaatacgaccgtatcatggaatacatcgaatcagggaaaaaagatgcaaatcttgctttaggcggcgttcgcaaagggaatgaggggtatttcattgagccaactatttttacagacgtgccgcacgacgcgaagattgccaaagaggagatcttcggtccagtggttgttgtgtcgaaatttaaggacgaaaaagatctgatccgtatcgcaaatgattctatttatggtttagctgcggcagtcttttcccgcgacatcagccgcgcgatcgagacagcacacaaactgaaagcaggcacggtctgggtcaactgctataatcagcttattccgcaggtgccattcggagggtataaggcttccggtatcggccgtgagttgggggaatatgccttgtctaattacacaaatatcaaggccgtccacgttaacctttctcaaccggcgcccatttga

SEQ ID NO: 40 - DNA, 1080 bp, Artificial Sequence
  Synthetic: trpDH
taagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtga

SEQ ID NO: 41 - DNA, 1674 bp, Artificial Sequence
  Synthetic: ipdC
gaaggagatatacatatgcgtacaccctactgtgtcgccgattatcttttagatcgtctgacggactgcggggccgatcacctgtttggcgtaccgggcgattacaacttgcagtttctggaccacgtcattgactcaccagatatctgctgggtagggtgtgcgaacgagcttaacgcgagctacgctgctgacggatatgcgcgttgtaaaggctttgctgcacttcttactaccttcggggtcggtgagttatcggcgatgaacggtatcgcaggctcgtacgctgagcacgtcccggtattacacattgtgggagctccgggtaccgcagctcaacagcgcggagaactgttacaccacacgctgggcgacggagaattccgccacttttaccatatgtccgagccaattactgtagcccaggctgtacttacagagcaaaatgcctgttacgagatcgaccgtgttttgaccacgatgcttcgcgagcgccgtcccgggtatttgatgctgccagccgatgttgccaaaaaagctgcgacgcccccagtgaatgccctgacgcataaacaagctcatgccgattccgcctgtttaaaggcttttcgcgatgcagctgaaaataaattagccatgtcgaaacgcaccgccttgttggcggactttctggtcctgcgccatggccttaaacacgcccttcagaaatgggtcaaagaagtcccgatggcccacgctacgatgcttatgggtaaggggatttttgatgaacgtcaagcgggattttatggaacttattccggttcggcgagtacgggggcggtaaaggaagcgattgagggagccgacacagttctttgcgtggggacacgtttcaccgatacactgaccgctggattcacacaccaacttactccggcacaaacgattgaggtgcaaccccatgcggctcgcgtgggggatgtatggtttacgggcattccaatgaatcaagccattgagactcttgtcgagctgtgcaaacagcacgtccacgcaggactgatgagttcgagctctggggcgattccttttccacaaccagatggtagtttaactcaagaaaacttctggcgcacattgcaaacctttatccgcccaggtgatatcatcttagcagaccagggtacttcagcctttggagcaattgacctgcgcttaccagcagacgtgaactttattgtgcagccgctgtgggggtctattggttatactttagctgcggccttcggagcgcagacagcgtgtccaaaccgtcgtgtgatcgtattgacaggagatggagcagcgcagttgaccattcaggagttaggctcgatgttacgcgataagcagcaccccattatcctggtcctgaacaatgaggggtatacagttgaacgcgccattcatggtgcggaacaacgctacaatgacatcgctttatggaattggacgcacatcccccaagccttatcgttagatccccaatcggaatgttggcgtgtgtctgaagcagagcaactggctgatgttctggaaaaagttgctcatcatgaacgcctgtcgttgatcgaggtaatgttgcccaaggccgatatccctccgttactgggagccttgaccaaggctttagaagcctgcaacaacgcttaa

SEQ ID NO: 42 - DNA, 1509 bp, Artificial Sequence
  Synthetic: Iad1
gaaggagatatacatatgcccaccttgaacttggacttacccaacggtattaagagcacgattcaggcagaccttttcatcaataataagtttgtgccggcgcttgatgggaaaacgttcgcaactattaatccgtctacggggaaagagatcggacaggtggcagaggcttcggcgaaggatgtggatcttgcagttaaggccgcgcgtgaggcgtttgaaactacttggggggaaaacacgccaggtgatgctcgtggccgtttactgattaagcttgctgagttggtggaagcgaatattgatgagttagcggcaattgaatcactggacaatgggaaagcgttctctattgctaagtcattcgacgtagctgctgtggccgcaaacttacgttactacggcggttgggctgataaaaaccacggtaaagtcatggaggtagacacaaagcgcctgaactatacccgccacgagccgatcggggtttgcggacaaatcattccgtggaatttcccgcttttgatgtttgcatggaagctgggtcccgctttagccacagggaacacaattgtgttaaagactgccgagcagactcccttaagtgctatcaagatgtgtgaattaatcgtagaagccggctttccgcccggagtagttaatgtgatctcgggattcggaccggtggcgggggccgcgatctcgcaacacatggacatcgataagattgcctttacaggatcgacattggttggccgcaacattatgaaggcagctgcgtcgactaacttaaaaaaggttacacttgagttaggaggaaaatccccgaatatcattttcaaagatgccgaccttgaccaagctgttcgctggagcgccttcggtatcatgtttaaccacggacaatgctgctgcgctggatcgcgcgtatatgtggaagaatccatctatgacgccttcatggaaaaaatgactgcgcattgtaaggcgcttcaagttggagatcctttcagcgcgaacaccttccaaggaccacaagtctcgcagttacaatacgaccgtatcatggaatacatcgaatcagggaaaaaagatgcaaatcttgctttaggcggcgttcgcaaagggaatgaggggtatttcattgagccaactatttttacagacgtgccgcacgacgcgaagattgccaaagaggagatcttcggtccagtggttgttgtgtcgaaatttaaggacgaaaaagatctgatccgtatcgcaaatgattctatttatggtttagctgcggcagtcttttcccgcgacatcagccgcgcgatcgagacagcacacaaactgaaagcaggcacggtctgggtcaactgctataatcagcttattccgcaggtgccattcggagggtataaggcttccggtatcggccgtgagttgggggaatatgccttgtctaattacacaaatatcaaggccgtccacgttaacctttctcaaccggcgcccatttga

SEQ ID NO: 43 - DNA, 6573 bp, Artificial Sequence
  Synthetic: TrpEDCBA
ctctagaaataattttgtttaactttaagaaggagatatacatatgcaaacacaaaaaccgactctcgaactgctaacctgcgaaggcgcttatcgcgacaacccgactgcgctttttcaccagttgtgtggggatcgtccggcaacgctgctgctggaatccgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttctaatggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagtta

SEQ ID NO: 44 - DNA, 1562 bp, Artificial Sequence
  Synthetic: trpE
atgcaaacacaaaaaccgactctcgaactgctaacctgcgaaggcgcttatcgcgacaacccgactgcgctttttcaccagttgtgtggggatcgtccggcaacgctgctgctggaatccgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttcta

SEQ ID NO: 45 - DNA, 1596 bp, Artificial Sequence
  Synthetic: trpD
atggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaa

SEQ ID NO: 46 - DNA, 355 bp, Artificial Sequence
  Synthetic: katG
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 47 - DNA, 228 bp, Artificial Sequence
  Synthetic: dps
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 48 - DNA, 334 bp, Artificial Sequence
  Synthetic: ahpC
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 49 - DNA, 134 bp, Artificial Sequence
  Synthetic: oxyS
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 50 - DNA, 36 bp, Artificial Sequence
  Synthetic: SR43
atatcgtcgcagcccacagcaacacgtttcctgagg

SEQ ID NO: 51 - DNA, 47 bp, Artificial Sequence
  Synthetic: SR44
aagaatttaacggagggcaaaaaaaaccgacgcacactggcgtcggc

SEQ ID NO: 52 - DNA, 1359 bp, Artificial Sequence
  Synthetic: trpC
atgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaa

SEQ ID NO: 53 - DNA, 1193 bp, Artificial Sequence
  Synthetic: trpB
atgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctg

SEQ ID NO: 54 - DNA, 807 bp, Artificial Sequence
  Synthetic: trpA
atggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagttaa

SEQ ID NO: 55 - DNA, 6574 bp, Artificial Sequence
  Synthetic: fbrS40FTrpE-DCBA
ctctagaaataattttgtttaactttaagaaggagatatacatatgcaaacacaaaaaccgactctcgaactgctaacctgcgaaggcgcttatcgcgacaacccgactgcgctttttcaccagttgtgtggggatcgtccggcaacgctgctgctggaattcgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttctaatggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaaatgatgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaaggaaaggaacaatgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctgatggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagttaa

SEQ ID NO: 56 - DNA, 1562 bp, Artificial Sequence
  Synthetic: fbrTrpE
atgcaaacacaaaaaccgactctcgaactgctaacctgcgaaggcgcttatcgcgacaacccgactgcgctttttcaccagttgtgtggggatcgtccggcaacgctgctgctggaattcgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttcta

SEQ ID NO: 57 - DNA, 7615 bp, Artificial Sequence
  Synthetic: fbrAroG-trpDH-fldABCDacuIfldH
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatgaaggttaagaaggagatatacatatgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtgagctttaagaaggagatatacatatgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttgacgcttaagaaggagatatacatatgcgtgctgtcttaatcgagaagtcagatgacacccagagtgtttcagttacggagttggctgaagaccaattacccgaaggtgacgtccttgtggatgtcgcgtacagcacattgaattacaaggatgctcttgcgattactggaaaagcacccgttgtacgccgttttcctatggtccccggaattgactttactgggactgtcgcacagagttcccatgctgatttcaagccaggcgaccgcgtaattctgaacggatggggagttggtgagaaacactggggcggtcttgcagaacgcgcacgcgtacgtggggactggcttgtcccgttgccagcccccttagacttgcgccaggctgcaatgattggcactgcggggtacacagctatgctgtgcgtgcttgcccttgagcgccatggagtcgtacctgggaacggcgagattgtcgtctcaggcgcagcaggaggggtaggttctgtagcaaccacactgttagcagccaaaggctacgaagtggccgccgtgaccgggcgcgcaagcgaggccgaatatttacgcggattaggcgccgcgtcggtcattgatcgcaatgaattaacggggaaggtgcgtccattagggcaggaacgctgggcaggaggaatcgatgtagcaggatcaaccgtacttgctaatatgttgagcatgatgaaataccgtggcgtggtggcggcctgtggcctggcggctggaatggacttgcccgcgtctgtcgccccttttattctgcgtggtatgactttggcaggggtagattcagtcatgtgccccaaaactgatcgtctggctgcttgggcacgcctggcatccgacctggaccctgcaaagctggaagagatgacaactgaattaccgttctctgaggtgattgaaacggctccgaagttcttggatggaacagtgcgtgggcgtattgtcattccggtaacaccttgatacttaagaaggagatatacatatgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagatt

SEQ ID NO: 58 - DNA, 1239 bp, Artificial Sequence
  Synthetic: fldA
atggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatga

SEQ ID NO: 59 - DNA, 1224 bp, Artificial Sequence
  Synthetic: fldB
atgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtga

SEQ ID NO: 60 - DNA, 1124 bp, Artificial Sequence
  Synthetic: fldC
atgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttg

SEQ ID NO: 61 - DNA, 981 bp, Artificial Sequence
  Synthetic: Acul
atgcgtgctgtcttaatcgagaagtcagatgacacccagagtgtttcagttacggagttggctgaagaccaattacccgaaggtgacgtccttgtggatgtcgcgtacagcacattgaattacaaggatgctcttgcgattactggaaaagcacccgttgtacgccgttttcctatggtccccggaattgactttactgggactgtcgcacagagttcccatgctgatttcaagccaggcgaccgcgtaattctgaacggatggggagttggtgagaaacactggggcggtcttgcagaacgcgcacgcgtacgtggggactggcttgtcccgttgccagcccccttagacttgcgccaggctgcaatgattggcactgcggggtacacagctatgctgtgcgtgcttgcccttgagcgccatggagtcgtacctgggaacggcgagattgtcgtctcaggcgcagcaggaggggtaggttctgtagcaaccacactgttagcagccaaaggctacgaagtggccgccgtgaccgggcgcgcaagcgaggccgaatatttacgcggattaggcgccgcgtcggtcattgatcgcaatgaattaacggggaaggtgcgtccattagggcaggaacgctgggcaggaggaatcgatgtagcaggatcaaccgtacttgctaatatgttgagcatgatgaaataccgtggcgtggtggcggcctgtggcctggcggctggaatggacttgcccgcgtctgtcgccccttttattctgcgtggtatgactttggcaggggtagattcagtcatgtgccccaaaactgatcgtctggctgcttgggcacgcctggcatccgacctggaccctgcaaagctggaagagatgacaactgaattaccgttctctgaggtgattgaaacggctccgaagttcttggatggaacagtgcgtgggcgtattgtcattccggtaacaccttga

SEQ ID NO: 62 - DNA, 996 bp, Artificial Sequence
  Synthetic: fldH1
atgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagattgcgggcgcggctctggatagctatgagtatgagcaaggtgtctttcacaacaataagatgaatgaaattatgcaggatgataccttggaacgtctgaaatcttttcccaacgtcgtgatcacgccgcatttgggtttttatactgatgaggcggtttccaatatggtagagatcacactgatgaaccttcaggaattcgagttgaaaggaacctgtaagaaccagcgtgtttgtaaatga

SEQ ID NO: 63 - DNA, 8008 bp, Artificial Sequence
  Synthetic: fbrAroG-TrpDH-fldABCDH
ctctagaaataattttgtttaactttaagaaggagatatacatatgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcgggtaatacttaagaaggagatatacatatgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcgtgaaatgtaagaaggagatatacatatggaaaacaacaccaatatgttctctggagtgaaggtgatcgaactggccaactttatcgctgctccggcggcaggtcgcttctttgctgatgggggagcagaagtaattaagatcgaatctccagcaggcgacccgctgcgctacacggccccatcagaaggacgcccgctttctcaagaggaaaacacaacgtatgatttggaaaacgcgaataagaaagcaattgttctgaacttaaaatcggaaaaaggaaagaaaattcttcacgagatgcttgctgaggcagacatcttgttaacaaattggcgcacgaaagcgttagtcaaacaggggttagattacgaaacactgaaagagaagtatccaaaattggtatttgcacagattacaggatacggggagaaaggacccgacaaagacctgcctggtttcgactacacggcgtttttcgcccgcggaggagtctccggtacattatatgaaaaaggaactgtccctcctaatgtggtaccgggtctgggtgaccaccaggcaggaatgttcttagctgccggtatggctggtgcgttgtataaggccaaaaccaccggacaaggcgacaaagtcaccgttagtctgatgcatagcgcaatgtacggcctgggaatcatgattcaggcagcccagtacaaggaccatgggctggtgtacccgatcaaccgtaatgaaacgcctaatcctttcatcgtttcatacaagtccaaagatgattactttgtccaagtttgcatgcctccctatgatgtgttttatgatcgctttatgacggccttaggacgtgaagacttggtaggtgacgaacgctacaataagatcgagaacttgaaggatggtcgcgcaaaagaagtctattccatcatcgaacaacaaatggtaacgaagacgaaggacgaatgggacaagatttttcgtgatgcagacattccattcgctattgcccaaacgtgggaagatcttttagaagacgagcaggcatgggccaacgactacctgtataaaatgaagtatcccacaggcaacgaacgtgccctggtacgtttacctgtgttcttcaaagaagctggacttcctgaatacaaccagtcgccacagattgctgagaataccgtggaagtgttaaaggagatgggatataccgagcaagaaattgaggagcttgagaaagacaaagacatcatggtacgtaaagagaaatgaaggttaagaaggagatatacatatgtcagaccgcaacaaagaagtgaaagaaaagaaggctaaacactatctgcgcgagatcacagctaaacactacaaggaagcgttagaggctaaagagcgtggggagaaagtgggttggtgtgcctctaacttcccccaagagattgcaaccacgttgggtgtaaaggttgtttatcccgaaaaccacgccgccgccgtagcggcacgtggcaatgggcaaaatatgtgcgaacacgcggaggctatgggattcagtaatgatgtgtgtggatatgcacgtgtaaatttagccgtaatggacatcggccatagtgaagatcaacctattccaatgcctgatttcgttctgtgctgtaataatatctgcaatcagatgattaaatggtatgaacacattgcaaaaacgttggatattcctatgatccttatcgatattccatataatactgagaacacggtgtctcaggaccgcattaagtacatccgcgcccagttcgatgacgctatcaagcaactggaagaaatcactggcaaaaagtgggacgagaataaattcgaagaagtgatgaagatttcgcaagaatcggccaagcaatggttacgcgccgcgagctacgcgaaatacaaaccatcaccgttttcgggctttgacctttttaatcacatggctgtagccgtttgtgctcgcggcacccaggaagccgccgatgcattcaaaatgttagcagatgaatatgaagagaacgttaagacaggaaagtctacttatcgcggcgaggagaagcagcgtatcttgttcgagggcatcgcttgttggccttatctgcgccacaagttgacgaaactgagtgaatatggaatgaacgtcacagctacggtgtacgccgaagcttttggggttatttacgaaaacatggatgaactgatggccgcttacaataaagtgcctaactcaatctccttcgagaacgcgctgaagatgcgtcttaatgccgttacaagcaccaatacagaaggggctgttatccacattaatcgcagttgtaagctgtggtcaggattcttatacgaactggcccgtcgtttggaaaaggagacggggatccctgttgtttcgttcgacggagatcaagcggatccccgtaacttctccgaggctcaatatgacactcgcatccaaggtttaaatgaggtgatggtcgcgaaaaaagaagcagagtgagctttaagaaggagatatacatatgtcgaatagtgacaagttttttaacgacttcaaggacattgtggaaaacccaaagaagtatatcatgaagcatatggaacaaacgggacaaaaagccatcggttgcatgcctttatacaccccagaagagcttgtcttagcggcgggtatgtttcctgttggagtatggggctcgaatactgagttgtcaaaagccaagacctactttccggcttttatctgttctatcttgcaaactactttagaaaacgcattgaatggggagtatgacatgctgtctggtatgatgatcacaaactattgcgattcgctgaaatgtatgggacaaaacttcaaacttacagtggaaaatatcgaattcatcccggttacggttccacaaaaccgcaagatggaggcgggtaaagaatttctgaaatcccagtataaaatgaatatcgaacaactggaaaaaatctcagggaataagatcactgacgagagcttggagaaggctattgaaatttacgatgagcaccgtaaagtcatgaacgatttctctatgcttgcgtccaagtaccctggtatcattacgccaacgaaacgtaactacgtgatgaagtcagcgtattatatggacaagaaagaacatacagagaaggtacgtcagttgatggatgaaatcaaggccattgagcctaaaccattcgaaggaaaacgcgtgattaccactgggatcattgcagattcggaggaccttttgaaaatcttggaggagaataacattgctatcgtgggagatgatattgcacacgagtctcgccaataccgcactttgaccccggaggccaacacacctatggaccgtcttgctgaacaatttgcgaaccgcgagtgttcgacgttgtatgaccctgaaaaaaaacgtggacagtatattgtcgagatggcaaaagagcgtaaggccgacggaatcatcttcttcatgacaaaattctgcgatcccgaagaatacgattaccctcagatgaaaaaagacttcgaagaagccggtattccccacgttctgattgagacagacatgcaaatgaagaactacgaacaagctcgcaccgctattcaagcattttcagaaaccctttgacgcttaagaaggagatatacatatgttctttacggagcaacacgaacttattcgcaaactggcgcgtgactttgccgaacaggaaatcgagcctatcgcagacgaagtagataaaaccgcagagttcccaaaagaaatcgtgaagaagatggctcaaaatggatttttcggcattaaaatgcctaaagaatacggaggggcgggtgcggataaccgcgcttatgtcactattatggaggaaatttcacgtgcttccggggtagcgggtatctacctgagctcgccgaacagtttgttaggaactcccttcttattggtcggaaccgatgagcaaaaagaaaagtaccttaagcctatgatccgcggcgagaagactctggcgttcgccctgacagagcctggtgctggctctgatgcgggtgcgttggctactactgcccgtgaagagggcgactattatatcttaaatggccgcaagacgtttattacaggggctcctattagcgacaatattattgtgttcgcaaaaaccgatatgagcaaagggaccaaaggtatcaccactttcattgtggactcaaagcaggaaggggtaagttttggtaagccagaggacaaaatgggaatgattggttgtccgacaagcgacatcatcttggaaaacgttaaagttcataagtccgacatcttgggagaagtcaataaggggtttattaccgcgatgaaaacactttccgttggtcgtatcggagtggcgtcacaggcgcttggaattgcacaggccgccgtagatgaggcggtaaagtacgccaagcaacgtaaacaattcaatcgcccaatcgcgaaatttcaggccattcaatttaaacttgccaatatggagactaaattaaatgccgctaaacttcttgtttataacgcagcgtacaaaatggattgtggagaaaaagccgacaaggaagcctctatggctaaatactttgctgctgaatcagcgatccaaatcgttaacgacgcgctgcaaatccatggcgggtatggctatatcaaagactacaagattgaacgtttgtaccgcgatgtgcgtgtgatcgctatttatgagggcacttccgaggtccaacagatggttatcgcgtccaatctgctgaagtaatacttaagaaggagatatacatatgaaaatcttggcatactgcgtccgcccagacgaggtagactcctttaagaaatttagtgaaaagtacgggcatacagttgatcttattccagactcttttggacctaatgtcgctcatttggcgaagggttacgatgggatttctattctgggcaacgacacgtgtaaccgtgaggcactggagaagatcaaggattgcgggatcaaatatctggcaacccgtacagccggagtgaacaacattgacttcgatgcagcaaaggagttcggtattaacgtggctaatgttcccgcatattcccccaactcggtcagcgaatttaccattggattggcattaagtctgacgcgtaagattccatttgccctgaaacgcgtggaactgaacaattttgcgcttggcggccttattggtgtggaattgcgtaacttaactttaggagtcatcggtactggtcgcatcggattgaaagtgattgagggcttctctgggtttggaatgaaaaaaatgatcggttatgacatttttgaaaatgaagaagcaaagaagtacatcgaatacaaatcattagacgaagtttttaaagaggctgatattatcactctgcatgcgcctctgacagacgacaactatcatatgattggtaaagaatccattgctaaaatgaaggatggggtatttattatcaacgcagcgcgtggagccttaatcgatagtgaggccctgattgaagggttaaaatcggggaagattgcgggcgcggctctggatagctatgagtatgagcaaggtgtctttcacaacaataagatgaatgaaattatgcaggatgataccttggaacgtctgaaatcttttcccaacgtcgtgatcacgccgcatttgggtttttatactgatgaggcggtttccaatatggtagagatcacactgatgaaccttcaggaattcgagttgaaaggaacctgtaagaaccagcgtgtttgtaaatga

SEQ ID NO: 64 - DNA, 1134 bp, Artificial Sequence
  Synthetic: FldD
atgttctttacggagcaacacgaacttattcgcaaactggcgcgtgactttgccgaacaggaaatcgagcctatcgcagacgaagtagataaaaccgcagagttcccaaaagaaatcgtgaagaagatggctcaaaatggatttttcggcattaaaatgcctaaagaatacggaggggcgggtgcggataaccgcgcttatgtcactattatggaggaaatttcacgtgcttccggggtagcgggtatctacctgagctcgccgaacagtttgttaggaactcccttcttattggtcggaaccgatgagcaaaaagaaaagtaccttaagcctatgatccgcggcgagaagactctggcgttcgccctgacagagcctggtgctggctctgatgcgggtgcgttggctactactgcccgtgaagagggcgactattatatcttaaatggccgcaagacgtttattacaggggctcctattagcgacaatattattgtgttcgcaaaaaccgatatgagcaaagggaccaaaggtatcaccactttcattgtggactcaaagcaggaaggggtaagttttggtaagccagaggacaaaatgggaatgattggttgtccgacaagcgacatcatcttggaaaacgttaaagttcataagtccgacatcttgggagaagtcaataaggggtttattaccgcgatgaaaacactttccgttggtcgtatcggagtggcgtcacaggcgcttggaattgcacaggccgccgtagatgaggcggtaaagtacgccaagcaacgtaaacaattcaatcgcccaatcgcgaaatttcaggccattcaatttaaacttgccaatatggagactaaattaaatgccgctaaacttcttgtttataacgcagcgtacaaaatggattgtggagaaaaagccgacaaggaagcctctatggctaaatactttgctgctgaatcagcgatccaaatcgttaacgacgcgctgcaaatccatggcgggtatggctatatcaaagactacaagattgaacgtttgtaccgcgatgtgcgtgtgatcgctatttatgagggcacttccgaggtccaacagatggttatcgcgtccaatctgctgaagtaa

SEQ ID NO: 65 - DNA, 117 bp, Artificial Sequence
  Synthetic: FNR-responsive regulatory region
atccccatcactcttgatggagatcaattccccaagctgctagagcgttaccttgcccttaaacattagcaatgtcgatttatcagagggccgacaggctcccacaggagaaaaccg

SEQ ID NO: 66 - DNA, 108 bp, Artificial Sequence
  Synthetic: FNR-responsive regulatory region
ctcttgatcgttatcaattcccacgctgtttcagagcgttaccttgcccttaaacattagcaatgtcgatttatcagagggccgacaggctcccacaggagaaaaccg

SEQ ID NO: 67 - DNA, 433 bp, Artificial Sequence
  Synthetic: FNR-responsive regulatory region, nirB2
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 68 - DNA, 290 bp, Artificial Sequence
  Synthetic: FNR-responsive regulatory region, nirB3
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 69 - DNA, 207 bp, Artificial Sequence
  Synthetic: FNR-responsive regulatory region, fnrS2
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 70 - DNA, 390 bp, Artificial Sequence
  Synthetic: FNR-responsive regulatory region, Pfnr6 nirB+crp
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 71 - DNA, 200 bp, Artificial Sequence
  Synthetic: FNR-responsive regulatory region, Pfnr7 fnrS+crp
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 72-95 - skipped (24 entries)

SEQ ID NO: 96 - DNA, 1470 bp, Artificial Sequence
  Synthetic: Tryptophan Decarboxylase (EC 4.1.1.28) Chain A, Ruminococcus Gnavus Tryptophan Decarboxylase Rumgna_01526 (alpha-fmt); codon optimized for the expression in E. coli
atgagtcaagtgattaagaagaaacgtaacacctttatgatcggaacggagtacattcttaacagtacacaattggaggaagcgattaaatcattcgtacatgatttctgcgcagagaagcatgagatccatgatcaacctgtggtagtagaagctaaagaacatcaggaggacaaaatcaaacaaatcaaaatcccggaaaagggacgtcctgtaaatgaagtcgtttctgagatgatgaatgaagtgtatcgctaccgcggagacgccaaccatcctcgctttttttcttttgtgcccggacctgcaagcagtgtgtcgtggttgggggatattatgacgtccgcctacaatattcatgctggaggctcaaagctggcaccgatggttaactgcattgagcaggaagttctgaagtggttagcaaagcaagtggggttcacagaaaatccaggtggcgtatttgtgtcgggcggttcaatggcgaatattacggcacttactgcggctcgtgacaataaactgaccgacattaaccttcatttgggaactgcttatattagtgaccagactcatagttcagttgcgaaaggattacgcattattggaatcactgacagtcgcatccgtcgcattcccactaactcccacttccagatggataccaccaagctggaggaagccatcgagaccgacaagaagtctggctacattccgttcgtcgttatcggaacagcaggtaccaccaacactggttcgattgaccccctgacagaaatctctgcgttatgtaagaagcatgacatgtggtttcatatcgacggagcgtatggagctagtgttctgctgtcacctaagtacaagagccttcttaccggaaccggcttggctgacagtatttcgtgggatgctcataaatggttgttccaaacgtacggctgtgcaatggtacttgtcaaagatatccgtaatttattccactcttttcatgtgaatcccgagtatcttaaggatctggaaaacgacatcgataacgttaatacatgggacatcggcatggagctgacgcgccctgcacgcggtcttaaattgtggcttactttacaggtccttggatctgacttgattgggagtgccattgaacacggtttccagctggcagtttgggctgaggaagcattgaatccaaagaaagactgggagatcgtttctccagctcagatggctatgattaatttccgttatgcccctaaggatttaaccaaagaggaacaggatattctgaatgaaaagatctcccaccgcattttagagagcggatacgctgcaattttcactactgtattaaacggcaagaccgttttacgcatctgtgcaattcacccggaggcaactcaagaggatatgcaacacacaatcgacttattagaccaatacggtcgtgaaatctataccgagatgaagaaagcg

SEQ ID NO: 97 - protein, 444 aa, Cryptococcus deuterogattii R265
  misc_feature (1)..(444): Trp aminotransferase (EC 2.6.1.27); tryptophan aminotransferase [Cryptococcus deuterogattii R265]
MTATTISIETVPQAPAAGTKTNGTSGKYNPRTYLSDRAKVTEIDGSDAGRPNPDTFPFNSITLNLKPPLGLPESSNNMPVSITIEDPDLATALQYAPSAGIPKLREWLADLQAHVHERPRGDYAISVGSGSQDLMFKGFQAVLNPGDPVLLETPMYSGVLPALRILKADYAEVDVDDQGLSAKNLEKVLSEWPADKKRPRVLYTSPIGSNPSGCSASKERKLEVLKVCKKYDVLIFEDDPYYYLAQELIPSYFALEKQVYPEGGHVVRFDSFSKLLSAGMRLGFATGPKEILHAIDVSTAGANLHTSAVSQGVALRLMQYWGIEGFLAHGRAVAKLYTERRAQFEATAHKYLDGLATWVSPVAGMFLWIDLRPAGIEDSYELIRHEALAKGVLGVPGMAFYPTGRKSSHVRVSFSIVDLEDESDLGFQRLAEAIKDKRKALGLA

SEQ ID NO: 98 - DNA, 1332 bp, Artificial Sequence
  Synthetic: Trp aminotransferase (EC 2.6.1.27); tryptophan aminotransferase [Cryptococcus deuterogattii R265], codon optimized for expression in E. coli
atgacggcaactacaatttctattgagaccgtacctcaggccccggcggcggggaccaaaactaatgggacttcaggaaaatacaacccccgcacttacctgtccgaccgcgccaaagtcactgagattgatggatctgacgccggtcgccccaatcccgatactttcccatttaactcgattaccttaaatttgaaaccacctttaggcttgcccgagagttcaaataacatgccggtctctatcacgattgaagaccccgatttagcgacggccttacaatatgcacctagcgccggtattcctaagctgcgcgaatggctggctgacttacaagctcacgttcatgagcgcccccgtggcgattatgccatctcggtcgggtcggggtcacaggatttgatgtttaagggcttccaagctgtcttgaatccaggtgatccagtccttctggaaaccccaatgtattcaggtgttctgccagcgctgcgcattctgaaggcggattatgcagaagttgatgtagacgaccaggggttatctgctaaaaaccttgaaaaagttttatcagagtggcccgcagataagaagcgtcctcgtgtcctgtatacgtcgccaatcggctccaatccttccggatgttcagcatccaaggaacgcaagttagaggtactgaaagtctgtaagaagtacgatgtgctgatcttcgaagacgatccgtattattaccttgctcaagagcttattccatcctattttgcgttggaaaaacaagtttatccggagggtgggcacgttgtacgctttgactcatttagtaaattgctttctgctgggatgcgcttgggatttgctacagggccgaaggaaattcttcatgcgattgacgtcagtacagcaggcgcaaatttacatacttcagcggtctctcaaggtgtcgctcttcgcctgatgcagtattgggggatcgagggattccttgcacatggccgcgcggtggccaaactttacacggagcgccgcgctcagttcgaggcaaccgcacataagtacctggacgggctggccacttgggtatctcccgtagcgggaatgtttttatggatcgatcttcgtccagcaggaatcgaagattcttacgaattaattcgccatgaagcattagccaaaggcgttttaggcgttccagggatggcgttttatccgacaggccgtaagtcttcccatgttcgtgtcagtttcagtatcgtcgacctggaagacgaatctgaccttggttttcaacgcctggctgaagctattaaggataaacgcaaggctttagggctggct

SEQ ID NO: 99 - protein, 500 aa, Catharanthus roseus
  misc_feature (1)..(500): TDC: Tryptophan decarboxylase from Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 100 - protein, 757 aa, E. coli
  misc_feature (1)..(757): TYNA: Monoamine oxidase from E. coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK

SEQ ID NO: 101 - protein, 1368 aa, Arabidopsis thaliana
  misc_feature (1)..(1368): AAO1: Indole-3-acetaldehyde oxidase from Arabidopsis thaliana
MGEKAIDEDKVEAMKSSKTSLVFAINGQRFELELSSIDPSTTLVDFLRNKTPFKSVKLGCGEGGCGACVVLLSKYDPLLEKVDEFTISSCLTLLCSIDGCSITTSDGLGNSRVGFHAVHERIAGFHATQCGFCTPGMSVSMFSALLNADKSHPPPRSGFSNLTAVEAEKAVSGNLCRCTGYRPLVDACKSFAADVDIEDLGFNAFCKKGENRDEVLRRLPCYDHTSSHVCTFPEFLKKEIKNDMSLHSRKYRWSSPVSVSELQGLLEVENGLSVKLVAGNTSTGYYKEEKERKYERFIDIRKIPEFTMVRSDEKGVELGACVTISKAIEVLREEKNVSVLAKIATHMEKIANRFVRNTGTIGGNIMMAQRKQFPSDLATILVAAQATVKIMTSSSSQEQFTLEEFLQQPPLDAKSLLLSLEIPSWHSAKKNGSSEDSILLFETYRAAPRPLGNALAFLNAAFSAEVTEALDGIVVNDCQLVFGAYGTKHAHRAKKVEEFLTGKVISDEVLMEAISLLKDEIVPDKGTSNPGYRSSLAVTFLFEFFGSLTKKNAKTTNGWLNGGCKEIGFDQNVESLKPEAMLSSAQQIVENQEHSPVGKGITKAGACLQASGEAVYVDDIPAPENCLYGAFIYSTMPLARIKGIRFKQNRVPEGVLGIITYKDIPKGGQNIGTNGFFTSDLLFAEEVTHCAGQIIAFLVADSQKHADIAANLVVIDYDTKDLKPPILSLEEAVENFSLFEVPPPLRGYPVGDITKGMDEAEHKILGSKISFGSQYFFYMETQTALAVPDEDNCMVVYSSTQTPEFVHQTIAGCLGVPENNVRVITRRVGGGFGGKAVKSMPVAAACALAASKMQRPVRTYVNRKTDMITTGGRHPMKVTYSVGFKSNGKITALDVEVLLDAGLTEDISPLMPKGIQGALMKYDWGALSFNVKVCKTNTVSRTALRAPGDVQGSYIGEAIIEKVASYLSVDVDEIRKVNLHTYESLRLFHSAKAGEFSEYTLPLLWDRIDEFSGFNKRRKVVEEFNASNKWRKRGISRVPAVYAVNMRSTPGRVSVLGDGSIVVEVQGIEIGQGLWTKVKQMAAYSLGLIQCGTTSDELLKKIRVIQSDTLSMVQGSMTAGSTTSEASSEAVRICCDGLVERLLPVKTALVEQTGGPVTWDSLISQAYQQSINMSVSSKYMPDSTGEYLNYGIAASEVEVNVLTGETTILRTDIIYDCGKSLNPAVDLGQIEGAFVQGLGFFMLEEFLMNSDGLVVTDSTWTYKIPTVDTIPRQFNVEILNSGQHKNRVLSSKASGEPPLLLAASVHCAVRAAVKEARKQILSWNSNKQGTDMYFELPVPATMPIVKEFCGLDVVEKYLEWKIQQRKNV

SEQ ID NO: 102 - protein, 513 aa, S. cerevisae
  misc_feature (1)..(513): ARO9: L-tryptophan aminotransferase from S. cerevisae
MTAGSAPPVDYTSLKKNFQPFLSRRVENRSLKSFWDASDISDDVIELAGGMPNERFFPIESMDLKISKVPFNDNPKWHNSFTTAHLDLGSPSELPIARSFQYAETKGLPPLLHFVKDFVSRINRPAFSDETESNWDVILSGGSNDSMFKVFETICDESTTVMIEEFTFTPAMSNVEATGAKVIPIKMNLTFDRESQGIDVEYLTQLLDNWSTGPYKDLNKPRVLYTIATGQNPTGMSVPQWKREKIYQLAQRHDFLIVEDDPYGYLYFPSYNPQEPLENPYHSSDLTTERYLNDFLMKSFLTLDTDARVIRLETFSKIFAPGLRLSFIVANKFLLQKILDLADITTRAPSGTSQAIVYSTIKAMAESNLSSSLSMKEAMFEGWIRWIMQIASKYNHRKNLTLKALYETESYQAGQFTVMEPSAGMFIIIKINWGNFDRPDDLPQQMDILDKFLLKNGVKVVLGYKMAVCPNYSKQNSDFLRLTIAYARDDDQLIEASKRIGSGIKEFFDNYKS

SEQ ID NO: 103 - protein, 396 aa, E. coli
  misc_feature (1)..(396): aspC: aspartate aminotransferase from E. coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 104 - protein, 391 aa, Arabidopsis thaliana
  misc_feature (1)..(391): TAA1: L-tryptophan-pyruvate aminotransferase from Arabidopsis thaliana
MVKLENSRKPEKISNKNIPMSDFVVNLDHGDPTAYEEYWRKMGDRCTVTIRGCDLMSYFSDMTNLCWFLEPELEDAIKDLHGVVGNAATEDRYIVVGTGSTQLCQAAVHALSSLARSQPVSVVAAAPFYSTYVEETTYVRSGMYKWEGDAWGFDKKGPYIELVTSPNNPDGTIRETVVNRPDDDEAKVIHDFAYYWPHYTPITRRQDHDIMLFTFSKITGHAGSRIGWALVKDKEVAKKMVEYIIVNSIGVSKESQVRTAKILNVLKETCKSESESENFFKYGREMMKNRWEKLREVVKESDAFTLPKYPEAFCNYFGKSLESYPAFAWLGTKEETDLVSELRRHKVMSRAGERCGSDKKHVRVSMLSREDVFNVFLERLANMKLIKSIDL

SEQ ID NO: 105 - protein, 504 aa, Streptomyces sp. TP-A0274
  misc_feature (1)..(504): STAO: L-tryptophan oxidase from streptomyces sp. TP-A0274
MTAPLQDSDGPDDAIGGPKQVTVIGAGIAGLVTAYELERLGHHVQIIEGSDDIGGRIHTHRFSGAGGPGPFAEMGAMRIPAGHRLTMHYIAELGLQNQVREFRTLFSDDAAYLPSSAGYLRVREAHDTLVDEFATGLPSAHYRQDTLLFGAWLDASIRAIAPRQFYDGLHNDIGVELLNLVDDIDLTPYRCGTARNRIDLHALFADHPRVRASCPPRLERFLDDVLDETSSSIVRLKDGMDELPRRLASRIRGKISLGQEVTGIDVHDDTVTLTVRQGLRTVTRTCDYVVCTIPFTVLRTLRLTGFDQDKLDIVHETKYWPATKIAFHCREPFWEKDGISGGASFTGGHVRQTYYPPAEGDPALGAVLLASYTIGPDAEALARMDEAERDALVAKELSVMHPELRRPGMVLAVAGRDWGARRWSRGAATVRWGQEAALREAERRECARPQKGLFFAGEHCSSKPAWIEGAIESAIDAAHEIEWYEPRASRVFAASRLSRSDRSA

SEQ ID NO: 106 - protein, 552 aa, Enterobacter cloacae
  misc_feature (1)..(552): ipdC: Indole-3-pyruvate decarboxylase from Enterobacter cloacae
MRTPYCVADYLLDRLTDCGADHLFGVPGDYNLQFLDHVIDSPDICWVGCANELNASYAADGYARCKGFAALLTTFGVGELSAMNGIAGSYAEHVPVLHIVGAPGTAAQQRGELLHHTLGDGEFRHFYHMSEPITVAQAVLTEQNACYEIDRVLTTMLRERRPGYLMLPADVAKKAATPPVNALTHKQAHADSACLKAFRDAAENKLAMSKRTALLADFLVLRHGLKHALQKWVKEVPMAHATMLMGKGIFDERQAGFYGTYSGSASTGAVKEAIEGADTVLCVGTRFTDTLTAGFTHQLTPAQTIEVQPHAARVGDVWFTGIPMNQAIETLVELCKQHVHAGLMSSSSGAIPFPQPDGSLTQENFWRTLQTFIRPGDIILADQGTSAFGAIDLRLPADVNFIVQPLWGSIGYTLAAAFGAQTACPNRRVIVLTGDGAAQLTIQELGSMLRDKQHPIILVLNNEGYTVERAIHGAEQRYNDIALWNWTHIPQALSLDPQSECWRVSEAEQLADVLEKVAHHERLSLIEVMLPKADIPPLLGALTKALEACNNA

SEQ ID NO: 107 - protein, 497 aa, Ustilago maydis
  misc_feature (1)..(497): IAD1: Indole-3-acetaldehyde dehydrogenase from Ustilago maydis
MPTLNLDLPNGIKSTIQADLFINNKFVPALDGKTFATINPSTGKEIGQVAEASAKDVDLAVKAAREAFETTWGENTPGDARGRLLIKLAELVEANIDELAAIESLDNGKAFSIAKSFDVAAVAANLRYYGGWADKNHGKVMEVDTKRLNYTRHEPIGVCGQIIPWNFPLLMFAWKLGPALATGNTIVLKTAEQTPLSAIKMCELIVEAGFPPGVVNVISGFGPVAGAAISQHMDIDKIAFTGSTLVGRNIMKAAASTNLKKVTLELGGKSPNIIFKDADLDQAVRWSAFGIMFNHGQCCCAGSRVYVEESIYDAFMEKMTAHCKALQVGDPFSANTFQGPQVSQLQYDRIMEYIESGKKDANLALGGVRKGNEGYFIEPTIFTDVPHDAKIAKEEIFGPVVVVSKFKDEKDLIRIANDSIYGLAAAVFSRDISRAIETAHKLKAGTVWVNCYNQLIPQVPFGGYKASGIGRELGEYALSNYTNIKAVHVNLSQPAPI

SEQ ID NO: 108 - protein, 415 aa, Arabidopsis thaliana
  misc_feature (1)..(415): YUC2: indole-3-pyruvate monoxygenase from Arabidopsis thaliana
MEFVTETLGKRIHDPYVEETRCLMIPGPIIVGSGPSGLATAACLKSRDIPSLILERSTCIASLWQHKTYDRLRLHLPKDFCELPLMPFPSSYPTYPTKQQFVQYLESYAEHFDLKPVFNQTVEEAKFDRRCGLWRVRTTGGKKDETMEYVSRWLVVATGENAEEVMPEIDGIPDFGGPILHTSSYKSGEIFSEKKILVVGCGNSGMEVCLDLCNFNALPSLVVRDSVHVLPQEMLGISTFGISTSLLKWFPVHVVDRFLLRMSRLVLGDTDRLGLVRPKLGPLERKIKCGKTPVLDVGTLAKIRSGHIKVYPELKRVMHYSAEFVDGRVDNFDAIILATGYKSNVPMWLKGVNMFSEKDGFPHKPFPNGWKGESGLYAVGFTKLGLLGAAIDAKKIAEDIEVQRHFLPLARPQHC

SEQ ID NO: 109 - protein, 557 aa, Pseudomonas savastanoi
  misc_feature (1)..(557): IaaM: Tryptophan 2-monooxygenase from Pseudomonas savastanoi
MYDHFNSPSIDILYDYGPFLKKCEMTGGIGSYSAGTPTPRVAIVGAGISGLVAATELLRAGVKDVVLYESRDRIGGRVWSQVFDQTRPRYIAEMGAMRFPPSATGLFHYLKKFGISTSTTFPDPGVVDTELHYRGKRYHWPAGKKPPELFRRVYEGWQSLLSEGYLLEGGSLVAPLDITAMLKSGRLEEAAIAWQGWLNVFRDCSFYNAIVCIFTGRHPPGGDRWARPEDFELFGSLGIGSGGFLPVFQAGFTEILRMVINGYQSDQRLIPDGISSLAARLADQSFDGKALRDRVCFSRVGRISREAEKIIIQTEAGEQRVFDRVIVTSSNRAMQMIHCLTDSESFLSRDVARAVRETHLTGSSKLFILTRTKFWIKNKLPTTIQSDGLVRGVYCLDYQPDEPEGHGVVLLSYTWEDDAQKMLAMPDKKTRCQVLVDDLAAIHPTFASYLLPVDGDYERYVLHHDWLTDPHSAGAFKLNYPGEDVYSQRLFFQPMTANSPNKDTGLYLAGCSCSFAGGWIEGAVQTALNSACAVLRSTGGQLSKGNPLDCINASYRY

SEQ ID NO: 110 - protein, 455 aa, Pseudomonas savastanoi
  misc_feature (1)..(455): iaaH: Indoleacetamide hydrolase from Pseudomonas savastanoi
MHEIITLESLCQALADGEIAAAELRERALDTEARLARLNCFIREGDAVSQFGEADHAMKGTPLWGMPVSFKDNICVRGLPLTAGTRGMSGFVSDQDAAIVSQLRALGAVVAGKNNMHELSFGVTSINPHWGTVGNPVAPGYCAGGSSGGSAAAVASGIVPLSVGTDTGGSIRIPAAFCGITGFRPTTGRWSTAGIIPVSHTKDCVGLLTRTAGDAGFLYGLLSGKQQSFPLSRTAPCRIGLPVSMWSDLDGEVERACVNALSLLRKTGFEFIEIDDADIVELNQTLTFTVPLYEFFADLAQSLLSLGWKHGIHHIFAQVDDANVKGIINHHLGEGAIKPAHYLSSLQNGELLKRKMDELFARHNIELLGYPTVPCRVPHLDHADRPEFFSQAIRNTDLASNAMLPSITIPVGPEGRLPVGLSFDALRGRDALLLSRVSAIEQVLGFVRKVLPHTT

SEQ ID NO: 111 - protein, 353 aa, Nostoc punctiforme NIES-2108
  misc_feature (1)..(353): TrpDH: Tryptophan dehydrogenase from Nostoc punctiforme NIES-2108
MLLFETVREMGHEQVLFCHSKNPEIKAIIAIHDTTLGPAMGATRILPYINEEAALKDALRLSRGMTYKAACANIPAGGGKAVIIANPENKTDDLLRAYGRFVDSLNGRFITGQDVNITPDDVRTISQETKYVVGVSEKSGGPAPITSLGVFLGIKAAVESRWQSKRLDGMKVAVQGLGNVGKNLCRHLHEHDVQLFVSDVDPIKAEEVKRLFGATVVEPTEIYSLDVDIFAPCALGGILNSHTIPFLQASIIAGAANNQLENEQLHSQMLAKKGILYSPDYVINAGGLINVYNEMIGYDEEKAFKQVHNIYDTLLAIFEIAKEQGVTTNDAARRLAEDRINNSKRSKSKAIAA

SEQ ID NO: 112 - protein, 541 aa, Arabidopsis thaliana
  misc_feature (1)..(541): CYP79B2: tryptophan N-monooxygenase from Arabidopsis thaliana
MNTFTSNSSDLTTTATETSSFSTLYLLSTLQAFVAITLVMLLKKLMTDPNKKKPYLPPGPTGWPIIGMIPTMLKSRPVFRWLHSIMKQLNTEIACVKLGNTHVITVTCPKIAREILKQQDALFASRPLTYAQKILSNGYKTCVITPFGDQFKKMRKVVMTELVCPARHRWLHQKRSEENDHLTAWVYNMVKNSGSVDFRFMTRHYCGNAIKKLMFGTRTFSKNTAPDGGPTVEDVEHMEAMFEALGFTFAFCISDYLPMLTGLDLNGHEKIMRESSAIMDKYHDPIIDERIKMWREGKRTQIEDFLDIFISIKDEQGNPLLTADEIKPTIKELVMAAPDNPSNAVEWAMAEMVNKPEILRKAMEEIDRVVGKERLVQESDIPKLNYVKAILREAFRLHPVAAFNLPHVALSDTTVAGYHIPKGSQVLLSRYGLGRNPKVWADPLCFKPERHLNECSEVTLTENDLRFISFSTGKRGCAAPALGTALTTMMLARLLQGFTWKLPENETRVELMESSHDMFLAKPLVMVGDLRLPEHLYPTVK

SEQ ID NO: 113 - protein, 543 aa, Arabidopsis thaliana
  misc_feature (1)..(543): CYP79B3: tryptophan N-monooxygenase from Arabidopsis thaliana
MDTLASNSSDLTTKSSLGMSSFTNMYLLTTLQALAALCFLMILNKIKSSSRNKKLHPLPPGPTGFPIVGMIPAMLKNRPVFRWLHSLMKELNTEIACVRLGNTHVIPVTCPKIAREIFKQQDALFASRPLTYAQKILSNGYKTCVITPFGEQFKKMRKVIMTEIVCPARHRWLHDNRAEETDHLTAWLYNMVKNSEPVDLRFVTRHYCGNAIKRLMFGTRTFSEKTEADGGPTLEDIEHMDAMFEGLGFTFAFCISDYLPMLTGLDLNGHEKIMRESSAIMDKYHDPIIDERIKMWREGKRTQIEDFLDIFISIKDEAGQPLLTADEIKPTIKELVMAAPDNPSNAVEWAIAEMINKPEILHKAMEEIDRVVGKERFVQESDIPKLNYVKAIIREAFRLHPVAAFNLPHVALSDTTVAGYHIPKGSQVLLSRYGLGRNPKVWSDPLSFKPERHLNECSEVTLTENDLRFISFSTGKRGCAAPALGTAITTMMLARLLQGFKWKLAGSETRVELMESSHDMFLSKPLVLVGELRLSEDLYPMVK

SEQ ID NO: 114 - protein, 503 aa, Arabidopis thaliana
  misc_feature (1)..(503): CYP71A13: indoleacetaldoxime dehydratase from Arabidopis thaliana
MSNIQEMEMILSISLCLTTLITLLLLRRFLKRTATKVNLPPSPWRLPVIGNLHQLSLHPHRSLRSLSLRYGPLMLLHFGRVPILVVSSGEAAQEVLKTHDHKFANRPRSKAVHGLMNGGRDVVFAPYGEYWRQMKSVCILNLLTNKMVESFEKVREDEVNAMIEKLEKASSSSSSENLSELFITLPSDVTSRVALGRKHSEDETARDLKKRVRQIMELLGEFPIGEYVPILAWIDGIRGFNNKIKEVSRGFSDLMDKVVQEHLEASNDKADFVDILLSIEKDKNSGFQVQRNDIKFMILDMFIGGTSTTSTLLEWTMTELIRSPKSMKKLQDEIRSTIRPHGSYIKEKEVENMKYLKAVIKEVLRLHPSLPMILPRLLSEDVKVKGYNIAAGTEVIINAWAIQRDTAIWGPDAEEFKPERHLDSGLDYHGKNLNYIPFGSGRRICPGINLALGLAEVTVANLVGRFDWRVEAGPNGDQPDLTEAIGIDVCRKFPLIAFPSSVV

SEQ ID NO: 115 - protein, 560 aa, Arabidopsis thaliana
  misc_feature (1)..(560): PEN2: myrosinase from Arabidopsis thaliana
MAHLQRTFPTEMSKGRASFPKGFLFGTASSSYQYEGAVNEGARGQSVWDHFSNRFPHRISDSSDGNVAVDFYHRYKEDIKRMKDINMDSFRLSIAWPRVLPYGKRDRGVSEEGIKFYNDVIDELLANEITPLVTIFHWDIPQDLEDEYGGFLSEQIIDDFRDYASLCFERFGDRVSLWCTMNEPWVYSVAGYDTGRKAPGRCSKYVNGASVAGMSGYEAYIVSHNMLLAHAEAVEVFRKCDHIKNGQIGIAHNPLWYEPYDPSDPDDVEGCNRAMDFMLGWHQHPTACGDYPETMKKSVGDRLPSFTPEQSKKLIGSCDYVGINYYSSLFVKSIKHVDPTQPTWRTDQGVDWMKTNIDGKQIAKQGGSEWSFTYPTGLRNILKYVKKTYGNPPILITENGYGEVAEQSQSLYMYNPSIDTERLEYIEGHIHAIHQAIHEDGVRVEGYYVWSLLDNFEWNSGYGVRYGLYYIDYKDGLRRYPKMSALWLKEFLRFDQEDDSSTSKKEEKKESYGKQLLHSVQDSQFVHSIKDSGALPAVLGSLFVVSATVGTSLFFKGANN

SEQ ID NO: 116 - protein, 346 aa, Arabidopsis thaliana
  misc_feature (1)..(346): Nit1: Nitrilase from Arabidopsis thaliana
MSSTKDMSTVQNATPFNGVAPSTTVRVTIVQSSTVYNDTPATIDKAEKYIVEAASKGAELVLFPEGFIGGYPRGFRFGLAVGVHNEEGRDEFRKYHASAIHVPGPEVARLADVARKNHVYLVMGAIEKEGYTLYCTVLFFSPQGQFLGKHRKLMPTSLERCIWGQGDGSTIPVYDTPIGKLGAAICWENRMPLYRTALYAKGIELYCAPTADGSKEWQSSMLHIAIEGGCFVLSACQFCQRKHFPDHPDYLFTDWYDDKEHDSIVSQGGSVIISPLGQVLAGPNFESEGLVTADIDLGDIARAKLYFDSVGHYSRPDVLHLTVNEHPRKSVTFVTKVEKAEDDSNK

SEQ ID NO: 117 - protein, 403 aa, homo sapiens [sequence omitted]

SEQ ID NO: 118 - protein, 406 aa, homo sapiens
  misc_feature (1)..(406): TDO2: tryptophan 2,3-dioxygenase from homo sapiens
MSGCPFLGNNFGYTFKKLPVEGSEEDKSQTGVNRASKGGLIYGNYLHLEKVLNAQELQSETKGNKIHDEHLFIITHQAYELWFKQILWELDSVREIFQNGHVRDERNMLKVVSRMHRVSVILKLLVQQFSILETMTALDFNDFREYLSPASGFQSLQFRLLENKIGVLQNMRVPYNRRHYRDNFKGEENELLLKSEQEKTLLELVEAWLERTPGLEPHGFNFWGKLEKNITRGLEEEFIRIQAKEESEEKEEQVAEFQKQKEVLLSLFDEKRHEHLLSKGERRLSYRALQGALMIYFYREEPRFQVPFQLLTSLMDIDSLMTKWRYNHVCMVHRMLGSKAGTGGSSGYHYLRSTVSDRYKVFVDLFNLSTYLIPRHWIPKMNPTIHKFLYTAEYCDSSYFSSDESD

SEQ ID NO: 119 - protein, 453 aa, S. cerevisiae
  misc_feature (1)..(453): BNA2: indoleamine 2,3-dioxygenase from S. cerevisiae
MNNTSITGPQVLHRTKMRPLPVLEKYCISPHHGFLDDRLPLTRLSSKKYMKWEEIVADLPSLLQEDNKVRSVIDGLDVLDLDETILGDVRELRRAYSILGFMAHAYIWASGTPRDVLPECIARPLLETAHILGVPPLATYSSLVLWNFKVTDECKKTETGCLDLENITTINTFTGTVDESWFYLVSVRFEKIGSACLNHGLQILRAIRSGDKGDANVIDGLEGLAATIERLSKALMEMELKCEPNVFYFKIRPFLAGWTNMSHMGLPQGVRYGAEGQYRIFSGGSNAQSSLIQTLDILLGVKHTANAAHSSQGDSKINYLDEMKKYMPREHREFLYHLESVCNIREYVSRNASNRALQEAYGRCISMLKIFRDNHIQIVTKYIILPSNSKQHGSNKPNVLSPIEPNTKASGCLGHKVASSKTIGTGGTRLMPFLKQCRDETVATADIKNEDKN

SEQ ID NO: 120 - protein, 305 aa, Mus musculus
  misc_feature (1)..(305): Afmid: Kynurenine formamidase from mouse
MAFPSLSAGQNPWRNLSSEELEKQYSPSRWVIHTKPEEVVGNFVQIGSQATQKARATRRNQLDVPYGDGEGEKLDIYFPDEDSKAFPLFLFLHGGYWQSGSKDDSAFMVNPLTAQGIVVVIVAYDIAPKGTLDQMVDQVTRSVVFLQRRYPSNEGIYLCGHSAGAHLAAMVLLARWTKHGVTPNLQGFLLVSGIYDLEPLIATSQNDPLRMTLEDAQRNSPQRHLDVVPAQPVAPACPVLVLVGQHDSPEFHRQSKEFYETLLRVGWKASFQQLRGVDHFDIIENLTREDDVLTQIILKTVFQKL

SEQ ID NO: 121 - protein, 444 aa, S. cerevisae
  misc_feature (1)..(444): BNA3: kynurenine--oxoglutarate transaminase from S. cerevisae
MKQRFIRQFTNLMSTSRPKVVANKYFTSNTAKDVWSLTNEAAAKAANNSKNQGRELINLGQGFFSYSPPQFAIKEAQKALDIPMVNQYSPTRGRPSLINSLIKLYSPIYNTELKAENVTVTTGANEGILSCLMGLLNAGDEVIVFEPFFDQYIPNIELCGGKVVYVPINPPKELDQRNTRGEEWTIDFEQFEKAITSKTKAVIINTPHNPIGKVFTREELTTLGNICVKHNVVIISDEVYEHLYFTDSFTRIATLSPEIGQLTLTVGSAGKSFAATGWRIGWVLSLNAELLSYAAKAHTRICFASPSPLQEACANSINDALKIGYFEKMRQEYINKFKIFTSIFDELGLPYTAPEGTYFVLVDFSKVKIPEDYPYPEEILNKGKDFRISHWLINELGVVAIPPTEFYIKEHEKAAENLLRFAVCKDDAYLENAVERLKLLKDYL

SEQ ID NO: 122 - protein, 430 aa, Homo sapiens
  misc_feature (1)..(430): GOT2: Aspartate aminotransferase, mitochondrial from homo sapiens
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 123 - protein, 425 aa, Unknown
  AADAT: Kynurenine/alpha-aminoadipate aminotransferase, mitochondrial
MNYARFITAASAARNPSPIRTMTDILSRGPKSMISLAGGLPNPNMFPFKTAVITVENGKTIQFGEEMMKRALQYSPSAGIPELLSWLKQLQIKLHNPPTIHYPPSQGQMDLCVTSGSQQGLCKVFEMIINPGDNVLLDEPAYSGTLQSLHPLGCNIINVASDESGIVPDSLRDILSRWKPEDAKNPQKNTPKFLYTVPNGNNPTGNSLTSERKKEIYELARKYDFLIIEDDPYYFLQFNKFRVPTFLSMDVDGRVIRADSFSKIISSGLRIGFLTGPKPLIERVILHIQVSTLHPSTFNQLMISQLLHEWGEEGFMAHVDRVIDFYSNQKDAILAAADKWLTGLAEWHVPAAGMFLWIKVKGINDVKELIEEKAVKMGVLMLPGNAFYVDSSAPSPYLRASFSSASPEQMDVAFQVLAQLIKESL

SEQ ID NO: 124 - protein, 422 aa, Homo sapiens
  misc_feature (1)..(422): CCLB1: Kynurenine--oxoglutarate transaminase 1 from homo sapiens
MAKQLQARRLDGIDYNPWVEFVKLASEHDVVNLGQGFPDFPPPDFAVEAFQHAVSGDFMLNQYTKTFGYPPLTKILASFFGELLGQEIDPLRNVLVTVGGYGALFTAFQALVDEGDEVIIIEPFFDCYEPMTMMAGGRPVFVSLKPGPIQNGELGSSSNWQLDPMELAGKFTSRTKALVLNTPNNPLGKVFSREELELVASLCQQHDVVCITDEVYQWMVYDGHQHISIASLPGMWERTLTIGSAGKTFSATGWKVGWVLGPDHIMKHLRTVHQNSVFHCPTQSQAAVAESFEREQLLFRQPSSYFVQFPQAMQRCRDHMIRSLQSVGLKPIIPQGSYFLITDISDFKRKMPDLPGAVDEPYDRRFVKWMIKNKGLVAIPVSIFYSVPHQKHFDHYIRFCFVKDEATLQAMDEKLRKWKVEL

SEQ ID NO: 125 - protein, 454 aa, Homo sapiens
  misc_feature (1)..(454): CCLB2: kynurenine--oxoglutarate transaminase 3 from homo sapiens
MFLAQRSLCSLSGRAKFLKTISSSKILGFSTSAKMSLKFTNAKRIEGLDSNVWIEFTKLAADPSVVNLGQGFPDISPPTYVKEELSKIAAIDSLNQYTRGFGHPSLVKALSYLYEKLYQKQIDSNKEILVTVGAYGSLFNTIQALIDEGDEVILIVPFYDCYEPMVRMAGATPVFIPLRSKPVYGKRWSSSDWTLDPQELESKFNSKTKAIILNTPHNPLGKVYNREELQVIADLCIKYDTLCISDEVYEWLVYSGNKHLKIATFPGMWERTITIGSAGKTFSVTGWKLGWSIGPNHLIKHLQTVQQNTIYTCATPLQEALAQAFWIDIKRMDDPECYFNSLPKELEVKRDRMVRLLESVGLKPIVPDGGYFIIADVSLLDPDLSDMKNNEPYDYKFVKWMTKHKKLSAIPVSAFCNSETKSQFEKFVRFCFIKKDSTLDAAEEIIKAWSVQKS

SEQ ID NO: 126 - protein, 471 aa, E. coli
  misc_feature (1)..(471): TnaA: tryptophanase from E. coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 127 - protein, 412 aa, Clostridium sporogenes
  misc_feature (1)..(412): FldA: indole-3-propionyl-CoA:indole-3-lactate CoA transferase from Clostridium sporogenes
MENNTNMFSGVKVIELANFIAAPAAGRFFADGGAEVIKIESPAGDPLRYTAPSEGRPLSQEENTTYDLENANKKAIVLNLKSEKGKKILHEMLAEADILLTNWRTKALVKQGLDYETLKEKYPKLVFAQITGYGEKGPDKDLPGFDYTAFFARGGVSGTLYEKGTVPPNVVPGLGDHQAGMFLAAGMAGALYKAKTTGQGDKVTVSLMHSAMYGLGIMIQAAQYKDHGLVYPINRNETPNPFIVSYKSKDDYFVQVCMPPYDVFYDRFMTALGREDLVGDERYNKIENLKDGRAKEVYSIIEQQMVTKTKDEWDKIFRDADIPFAIAQTWEDLLEDEQAWANDYLYKMKYPTGNERALVRLPVFFKEAGLPEYNQSPQIAENTVEVLKEMGYTEQEIEELEKDKDIMVRKEK

SEQ ID NO: 128 - protein, 407 aa, Clostridium sporogenes
  misc_feature (1)..(407): FldB: subunit of indole-3-lactate dehydratase from Clostridium sporogenes
MSDRNKEVKEKKAKHYLREITAKHYKEALEAKERGEKVGWCASNFPQEIATTLGVKVVYPENHAAAVAARGNGQNMCEHAEAMGFSNDVCGYARVNLAVMDIGHSEDQPIPMPDFVLCCNNICNQMIKWYEHIAKTLDIPMILIDIPYNTENTVSQDRIKYIRAQFDDAIKQLEEITGKKWDENKFEEVMKISQESAKQWLRAASYAKYKPSPFSGFDLFNHMAVAVCARGTQEAADAFKMLADEYEENVKTGKSTYRGEEKQRILFEGIACWPYLRHKLTKLSEYGMNVTATVYAEAFGVIYENMDELMAAYNKVPNSISFENALKMRLNAVTSTNTEGAVIHINRSCKLWSGFLYELARRLEKETGIPVVSFDGDQADPRNFSEAQYDTRIQGLNEVMVAKKEAE

SEQ ID NO: 129 - protein, 374 aa, Clostridium sporogenes
  misc_feature (1)..(374): FldC: subunit of indole-3-lactate dehydratase from Clostridium sporogenes
MSNSDKFFNDFKDIVENPKKYIMKHMEQTGQKAIGCMPLYTPEELVLAAGMFPVGVWGSNTELSKAKTYFPAFICSILQTTLENALNGEYDMLSGMMITNYCDSLKCMGQNFKLTVENIEFIPVTVPQNRKMEAGKEFLKSQYKMNIEQLEKISGNKITDESLEKAIEIYDEHRKVMNDFSMLASKYPGIITPTKRNYVMKSAYYMDKKEHTEKVRQLMDEIKAIEPKPFEGKRVITTGIIADSEDLLKILEENNIAIVGDDIAHESRQYRTLTPEANTPMDRLAEQFANRECSTLYDPEKKRGQYIVEMAKERKADGIIFFMTKFCDPEEYDYPQMKKDFEEAGIPHVLIETDMQMKNYEQARTAIQAFSETL

SEQ ID NO: 130 - protein, 377 aa, Clostridium sporogenes
  misc_feature (1)..(377): FldD: indole-3-acrylyl-CoA reductase from Clostridium sporogenes
MFFTEQHELIRKLARDFAEQEIEPIADEVDKTAEFPKEIVKKMAQNGFFGIKMPKEYGGAGADNRAYVTIMEEISRASGVAGIYLSSPNSLLGTPFLLVGTDEQKEKYLKPMIRGEKTLAFALTEPGAGSDAGALATTAREEGDYYILNGRKTFITGAPISDNIIVFAKTDMSKGTKGITTFIVDSKQEGVSFGKPEDKMGMIGCPTSDIILENVKVHKSDILGEVNKGFITAMKTLSVGRIGVASQALGIAQAAVDEAVKYAKQRKQFNRPIAKFQAIQFKLANMETKLNAAKLLVYNAAYKMDCGEKADKEASMAKYFAAESAIQIVNDALQIHGGYGYIKDYKIERLYRDVRVIAIYEGTSEVQQMVIASNLLK

SEQ ID NO: 131 - protein, 331 aa, Clostridium sporogenes
  misc_feature (1)..(331): FldH1: indole-3-lactate dehydrogenase from Clostridium sporogenes
MKILAYCVRPDEVDSFKKFSEKYGHTVDLIPDSFGPNVAHLAKGYDGISILGNDTCNREALEKIKDCGIKYLATRTAGVNNIDFDAAKEFGINVANVPAYSPNSVSEFTIGLALSLTRKIPFALKRVELNNFALGGLIGVELRNLTLGVIGTGRIGLKVIEGFSGFGMKKMIGYDIFENEEAKKYIEYKSLDEVFKEADIITLHAPLTDDNYHMIGKESIAKMKDGVFIINAARGALIDSEALIEGLKSGKIAGAALDSYEYEQGVFHNNKMNEIMQDDTLERLKSFPNVVITPHLGFYTDEAVSNMVEITLMNLQEFELKGTCKNQRVCK

SEQ ID NO: 132 - protein, 334 aa, Clostridium sporogenes
  misc_feature (1)..(334): FldH2: indole-3-lactate dehydrogenase from Clostridium sporogenes
MKILMYSVREHEKPAIKKWLEANPGVQIDLCNNALSEDTVCKAKEYDGIAIQQTNSIGGKAVYSTLKEYGIKQIASRTAGVDMIDLKMASDSNILVTNVPAYSPNAIAELAVTHTMNLLRNIKTLNKRIAYGDYRWSADLIAREVRSVTVGVVGTGKIGRTSAKLFKGLGANVIGYDAYPDKKLEENNLLTYKESLEDLLREADVVTLHTPLLESTKYMINKNNLKYMKPDAFIVNTGRGGIINTEDLIEALEQNKIAGAALDTFENEGLFLNKVVDPTKLPDSQLDKLLKMDQVLITHHVGFFTTTAVQNIVDTSLDSVVEVLKTNNSVNKVN

SEQ ID NO: 133 - protein, 326 aa, Rhodobacter sphaeroides
  misc_feature (1)..(326): AcuI: acrylyl-CoA reductase from Rhodobacter sphaeroides
MRAVLIEKSDDTQSVSVTELAEDQLPEGDVLVDVAYSTLNYKDALAITGKAPVVRRFPMVPGIDFTGTVAQSSHADFKPGDRVILNGWGVGEKHWGGLAERARVRGDWLVPLPAPLDLRQAAMIGTAGYTAMLCVLALERHGVVPGNGEIVVSGAAGGVGSVATTLLAAKGYEVAAVTGRASEAEYLRGLGAASVIDRNELTGKVRPLGQERWAGGIDVAGSTVLANMLSMMKYRGVVAACGLAAGMDLPASVAPFILRGMTLAGVDSVMCPKTDRLAAWARLASDLDPAKLEEMTTELPFSEVIETAPKFLDGTVRGRIVIPVTP

SEQ ID NO: 134-151 - skipped (18 entries)

SEQ ID NO: 152 - DNA, 30 bp, Artificial Sequence
  Synthetic: BBa_I14018, P(Bla)
gtttatacataggcgagtactctgttatgg

SEQ ID NO: 153 - DNA, 30 bp, Artificial Sequence
  Synthetic: BBa_I14033, P(Cat)
agaggttccaactttcaccataatgaaaca

SEQ ID NO: 154 - DNA, 30 bp, Artificial Sequence
  Synthetic: BBa_I14034, P(Kat)
taaacaactaacggacaattctacctaaca

SEQ ID NO: 155 - DNA, 30 bp, Artificial Sequence
  Synthetic: BBa_I732021, Template for Building Primer Family Member
acatcaagccaaattaaacaggattaacac

SEQ ID NO: 156 - DNA, 30 bp, Artificial Sequence
  Synthetic: BBa_I742126, Reverse lambda cI-regulated promoter
gaggtaaaatagtcaacacgcacggtgtta

SEQ ID NO: 157 - DNA, 30 bp, Artificial Sequence
  Synthetic: BBa_J01006, Key Promoter absorbs 3
caggccggaataactccctataatgcgcca

SEQ ID NO: 158 - DNA, 30 bp, Artificial Sequence
  Synthetic: BBa_J23100, constitutive promoter family member
ggctagctcagtcctaggtacagtgctagc

SEQ ID NO: 159 - DNA, 30 bp, Artificial Sequence
  Synthetic: BBa_J23101, constitutive promoter family member
agctagctcagtcctaggtattatgctagc

SEQ ID NO: 160 - DNA, 30 bp, Artificial Sequence
  Synthetic: BBa_J23102, constitutive promoter family member
agctagctcagtcctaggtactgtgctagc

SEQ ID NO: 161 - DNA, 30 bp, Artificial Sequence
  Synthetic: BBa_J23103, constitutive promoter family member
agctagctcagtcctagggattatgctagc

SEQ ID NO: 162 - DNA, 30 bp, Artificial Sequence
  Synthetic: BBa_J23104, constitutive promoter family member
agctagctcagtcctaggtattgtgctagc

SEQ ID NO: 163 - DNA, 30 bp, Artificial Sequence
  Synthetic: BBa_J23105, constitutive promoter family member
ggctagctcagtcctaggtactatgctagc

SEQ ID NO: 164 - DNA, 30 bp, Artificial Sequence
  Synthetic: BBa_J23106, constitutive promoter family member
ggctagctcagtcctaggtatagtgctagc

SEQ ID NO: 165 - DNA, 30 bp, Artificial Sequence
  Synthetic: BBa_J23107, constitutive promoter family member
ggctagctcagccctaggtattatgctagc

SEQ ID NO: 166 - DNA, 30 bp, Artificial Sequence
  Synthetic: BBa_J23108, constitutive promoter family member
agctagctcagtcctaggtataatgctagc

SEQ ID NO: 167 - DNA, 30 bp, Artificial Sequence
  Synthetic: BBa_J23109, constitutive promoter family member
agctagctcagtcctagggactgtgctagc

SEQ ID NO: 168 - DNA, 30 bp, Artificial Sequence
  Synthetic: BBa_J23110, constitutive promoter family member
ggctagctcagtcctaggtacaatgctagc

SEQ ID NO: 169 - DNA, 30 bp, Artificial Sequence; identical to SEQ ID NO: 164
  Synthetic: BBa_J23111, constitutive promoter family member

SEQ ID NO: 170 - DNA, 30 bp, Artificial Sequence; identical to SEQ ID NO: 161
  Synthetic: BBa_J23112, constitutive promoter family member

SEQ ID NO: 171 - DNA, 30 bp, Artificial Sequence
  Synthetic: BBa_J23113, constitutive promoter family member
ggctagctcagtcctagggattatgctagc

SEQ ID NO: 172 - DNA, 30 bp, Artificial Sequence; identical to SEQ ID NO: 168
  Synthetic: BBa_J23114, constitutive promoter family member

SEQ ID NO: 173 - DNA, 30 bp, Artificial Sequence
  Synthetic: BBa_J23115, constitutive promoter family member
agctagctcagcccttggtacaatgctagc

SEQ ID NO: 174 - DNA, 30 bp, Artificial Sequence
  Synthetic: BBa_J23116, constitutive promoter family member
agctagctcagtcctagggactatgctagc

SEQ ID NO: 175 - DNA, 30 bp, Artificial Sequence
  Synthetic: BBa_J23117, constitutive promoter family member
agctagctcagtcctagggattgtgctagc

SEQ ID NO: 176 - DNA, 30 bp, Artificial Sequence
  Synthetic: BBa_J23118, constitutive promoter family member
ggctagctcagtcctaggtattgtgctagc

SEQ ID NO: 177 - DNA, 30 bp, Artificial Sequence; identical to SEQ ID NO: 166
  Synthetic: BBa_J23119, constitutive promoter family member

SEQ ID NO: 178 - DNA, 30 bp, Artificial Sequence
  Synthetic: BBa_J23150, 1bp mutant from J23107
ggctagctcagtcctaggtattatgctagc

SEQ ID NO: 179 - DNA, 30 bp, Artificial Sequence; identical to SEQ ID NO: 168
  Synthetic: BBa_J23151, 1bp mutant from J23114

SEQ ID NO: 180 - DNA, 30 bp, Artificial Sequence
  Synthetic: BBa_J44002, pBAD reverse
aaagtgtgacgccgtgcaaataatcaatgt

SEQ ID NO: 181 - DNA, 30 bp, Artificial Sequence
  Synthetic: BBa_J48104, NikR promoter, a protein of the ribbon helix-helix family of trancription factors that repress expre
gacgaatacttaaaatcgtcatacttattt

SEQ ID NO: 182 - DNA, 30 bp, Artificial Sequence
  Synthetic: BBa_J54200, lacq_Promoter
aaacctttcgcggtatggcatgatagcgcc

SEQ ID NO: 183 - DNA, 30 bp, Artificial Sequence
  Synthetic: BBa_J56015, lacIQ - promoter sequence
tgatagcgcccggaagagagtcaattcagg

SEQ ID NO: 184 - DNA, 30 bp, Artificial Sequence
  Synthetic: BBa_J64951, E. Coli CreABCD phosphate sensing operon promoter
ttatttaccgtgacgaactaattgctcgtg

SEQ ID NO: 185 - DNA, 30 bp, Artificial Sequence
  Synthetic: BBa_K088007, GlnRS promoter
catacgccgttatacgttgtttacgctttg

SEQ ID NO: 186 - DNA, 30 bp, Artificial Sequence
  Synthetic: BBa_K119000, Constitutive weak promoter of lacZ
ttatgcttccggctcgtatgttgtgtggac

SEQ ID NO: 187 - DNA, 30 bp, Artificial Sequence
  Synthetic: BBa_K119001, Mutated LacZ promoter
ttatgcttccggctcgtatggtgtgtggac

SEQ ID NO: 188 - DNA, 30 bp, Artificial Sequence; identical to SEQ ID NO: 163
  Synthetic: BBa_K1330002, Constitutive promoter (J23105)

SEQ ID NO: 189 - DNA, 30 bp, Artificial Sequence
  Synthetic: BBa_K137029, constitutive promoter with (TA)10 between -10 and -35 elements
atatatatatatatataatggaagcgtttt

SEQ ID NO: 190 - DNA, 30 bp, Artificial Sequence; identical to SEQ ID NO: 189
  Synthetic: BBa_K137030, constitutive promoter with (TA)9 between -10 and -35 elements

SEQ ID NO: 191 - DNA, 30 bp, Artificial Sequence
  Synthetic: BBa_K137031, constitutive promoter with (C)10 between  -10 and -35 elements
ccccgaaagcttaagaatataattgtaagc

SEQ ID NO: 192 - DNA, 30 bp, Artificial Sequence; identical to SEQ ID NO: 191
  Synthetic: BBa_K137032, constitutive promoter with (C)12 between -10 and -35 elements

SEQ ID NO: 193 - DNA, 30 bp, Artificial Sequence
  Synthetic: BBa_K137085, optimized (TA) repeat constitutive promoter with 13 bp between -10 and -35 elements
tgacaatatatatatatatataatgctagc

SEQ ID NO: 194 - DNA, 30 bp, Artificial Sequence
  Synthetic: BBa_K137086, optimized (TA) repeat constitutive promoter with 15 bp between -10 and -35 elements
acaatatatatatatatatataatgctagc

SEQ ID NO: 195 - DNA, 30 bp, Artificial Sequence
  Synthetic: BBa_K137087, optimized (TA) repeat constitutive promoter with 17 bp between -10 and -35 elements
aatatatatatatatatatataatgctagc

SEQ ID NO: 196 - DNA, 30 bp, Artificial Sequence
  Synthetic: BBa_K137088, optimized (TA) repeat constitutive promoter with 19 bp between -10 and -35 elements
tatatatatatatatatatataatgctagc

SEQ ID NO: 197 - DNA, 30 bp, Artificial Sequence; identical to SEQ ID NO: 196
  Synthetic: BBa_K137089, optimized (TA) repeat constitutive promoter with 21 bp between -10 and -35 elements

SEQ ID NO: 198 - DNA, 30 bp, Artificial Sequence
  Synthetic: BBa_K137090, optimized (A) repeat constitutive promoter with 17 bp between -10 and -35 elements
aaaaaaaaaaaaaaaaaatataatgctagc

SEQ ID NO: 199 - DNA, 30 bp, Artificial Sequence; identical to SEQ ID NO: 198
  Synthetic: BBa_K137091, optimized (A) repeat constitutive promoter with 18 bp between -10 and -35 elements

SEQ ID NO: 200 - DNA, 30 bp, Artificial Sequence
  Synthetic: BBa_K1585100, Anderson Promoter with lacI binding site
ggaattgtgagcggataacaatttcacaca

SEQ ID NO: 201 - DNA, 30 bp, Artificial Sequence; identical to SEQ ID NO: 200
  Synthetic: BBa_K1585101, Anderson Promoter with lacI binding site

SEQ ID NO: 202 - DNA, 30 bp, Artificial Sequence; identical to SEQ ID NO: 200
  Synthetic: BBa_K1585102, Anderson Promoter with lacI binding site

SEQ ID NO: 203 - DNA, 30 bp, Artificial Sequence; identical to SEQ ID NO: 200
  Synthetic: BBa_K1585103, Anderson Promoter with lacI binding site

SEQ ID NO: 204 - DNA, 30 bp, Artificial Sequence; identical to SEQ ID NO: 200
  Synthetic: BBa_K1585104, Anderson Promoter with lacI binding site

SEQ ID NO: 205 - DNA, 30 bp, Artificial Sequence; identical to SEQ ID NO: 200
  Synthetic: BBa_K1585105, Anderson Promoter with lacI binding site

SEQ ID NO: 206 - DNA, 30 bp, Artificial Sequence; identical to SEQ ID NO: 200
  Synthetic: BBa_K1585106, Anderson Promoter with lacI binding site

SEQ ID NO: 207 - DNA, 30 bp, Artificial Sequence; identical to SEQ ID NO: 200
  Synthetic: BBa_K1585110, Anderson Promoter with lacI binding site

SEQ ID NO: 208 - DNA, 30 bp, Artificial Sequence; identical to SEQ ID NO: 200
  Synthetic: BBa_K1585113, Anderson Promoter with  lacI binding site

SEQ ID NO: 209 - DNA, 30 bp, Artificial Sequence; identical to SEQ ID NO: 200
  Synthetic: BBa_K1585115, Anderson Promoter with lacI binding site

SEQ ID NO: 210 - DNA, 30 bp, Artificial Sequence; identical to SEQ ID NO: 200
  Synthetic: BBa_K1585116, Anderson Promoter with lacI binding site

SEQ ID NO: 211 - DNA, 30 bp, Artificial Sequence; identical to SEQ ID NO: 200
  Synthetic: BBa_K1585117, Anderson Promoter with lacI binding site

SEQ ID NO: 212 - DNA, 30 bp, Artificial Sequence; identical to SEQ ID NO: 200
  Synthetic: BBa_K1585118, Anderson Promoter with lacI binding site

SEQ ID NO: 213 - DNA, 30 bp, Artificial Sequence; identical to SEQ ID NO: 200
  Synthetic: BBa_K1585119, Anderson Promoter with lacI binding site

SEQ ID NO: 214 - DNA, 30 bp, Artificial Sequence
  Synthetic: BBa_K1824896, J23100 + RBS
gattaaagaggagaaatactagagtactag

SEQ ID NO: 215 - DNA, 30 bp, Artificial Sequence
  Synthetic: BBa_K256002, J23101:GFP
caccttcgggtgggcctttctgcgtttata

SEQ ID NO: 216 - DNA, 30 bp, Artificial Sequence; identical to SEQ ID NO: 215
  Synthetic: BBa_K256018, J23119:IFP

SEQ ID NO: 217 - DNA, 30 bp, Artificial Sequence; identical to SEQ ID NO: 215
  Synthetic: BBa_K256020, J23119:HO1

SEQ ID NO: 218 - DNA, 30 bp, Artificial Sequence; identical to SEQ ID NO: 215
  Synthetic: BBa_K256033, Infrared signal reporter (J23119:IFP:J23119:HO1)

SEQ ID NO: 219 - DNA, 30 bp, Artificial Sequence; identical to SEQ ID NO: 158
  Synthetic: BBa_K292000, Double terminator + constitutive promoter

SEQ ID NO: 220 - DNA, 30 bp, Artificial Sequence
  Synthetic: BBa_K292001, Double terminator + Constitutive promoter + Strong RBS
tgctagctactagagattaaagaggagaaa

SEQ ID NO: 221 - DNA, 30 bp, Artificial Sequence
  Synthetic: BBa_K418000, IPTG inducible Lac promoter cassette
ttgtgagcggataacaagatactgagcaca

SEQ ID NO: 222 - DNA, 30 bp, Artificial Sequence; identical to SEQ ID NO: 221
  Synthetic: BBa_K418002, IPTG inducible Lac promoter cassette

SEQ ID NO: 223 - DNA, 30 bp, Artificial Sequence; identical to SEQ ID NO: 221
  Synthetic: BBa_K418003, IPTG inducible Lac promoter cassette

SEQ ID NO: 224 - DNA, 30 bp, Artificial Sequence; identical to SEQ ID NO: 158
  Synthetic: BBa_K823004, Anderson promoter J23100

SEQ ID NO: 225 - DNA, 30 bp, Artificial Sequence; identical to SEQ ID NO: 159
  Synthetic: BBa_K823005, Anderson promoter J23101

SEQ ID NO: 226 - DNA, 30 bp, Artificial Sequence; identical to SEQ ID NO: 160
  Synthetic: BBa_K823006, Anderson promoter J23102

SEQ ID NO: 227 - DNA, 30 bp, Artificial Sequence; identical to SEQ ID NO: 161
  Synthetic: BBa_K823007, Anderson promoter J23103

SEQ ID NO: 228 - DNA, 30 bp, Artificial Sequence; identical to SEQ ID NO: 164
  Synthetic: BBa_K823008, Anderson promoter J23106

SEQ ID NO: 229 - DNA, 30 bp, Artificial Sequence; identical to SEQ ID NO: 171
  Synthetic: BBa_K823010, Anderson promoter J23113

SEQ ID NO: 230 - DNA, 30 bp, Artificial Sequence; identical to SEQ ID NO: 168
  Synthetic: BBa_K823011, Anderson promoter J23114

SEQ ID NO: 231 - DNA, 30 bp, Artificial Sequence; identical to SEQ ID NO: 175
  Synthetic: BBa_K823013, Anderson promoter J23117

SEQ ID NO: 232 - DNA, 30 bp, Artificial Sequence; identical to SEQ ID NO: 176
  Synthetic: BBa_K823014, Anderson promoter J23118

SEQ ID NO: 233 - DNA, 30 bp, Artificial Sequence
  Synthetic: BBa_M13101, M13K07 gene I promoter
cctgtttttatgttattctctctgtaaagg

SEQ ID NO: 234 - DNA, 30 bp, Artificial Sequence
  Synthetic: BBa_M13102, M13K07 gene II promoter
aaatatttgcttatacaatcttcctgtttt

SEQ ID NO: 235 - DNA, 30 bp, Artificial Sequence
  Synthetic: BBa_M13103, M13K07 gene III promoter
gctgataaaccgatacaattaaaggctcct

SEQ ID NO: 236 - DNA, 30 bp, Artificial Sequence
  Synthetic: BBa_M13104, M13K07 gene IV promoter
ctcttctcagcgtcttaatctaagctatcg

SEQ ID NO: 237 - DNA, 30 bp, Artificial Sequence
  Synthetic: BBa_M13105, M13K07 gene V promoter
atgagccagttcttaaaatcgcataaggta

SEQ ID NO: 238 - DNA, 30 bp, Artificial Sequence
  Synthetic: BBa_M13106, M13K07 gene VI promoter
ctattgattgtgacaaaataaacttattcc

SEQ ID NO: 239 - DNA, 30 bp, Artificial Sequence
  Synthetic: BBa_M13108, M13K07 gene VIII promoter
gtttcgcgcttggtataatcgctgggggtc

SEQ ID NO: 240 - DNA, 30 bp, Artificial Sequence
  Synthetic: BBa_M13110, M13110
ctttgcttctgactataatagtcagggtaa

SEQ ID NO: 241 - DNA, 30 bp, Artificial Sequence
  Synthetic: BBa_M31519, Modified promoter sequence of g3.
aaaccgatacaattaaaggctcctgctagc

SEQ ID NO: 242 - DNA, 30 bp, Artificial Sequence
  Synthetic: BBa_R1074, Constitutive Promoter I
caccacactgatagtgctagtgtagatcac

SEQ ID NO: 243 - DNA, 30 bp, Artificial Sequence
  Synthetic: BBa_R1075, Constitutive Promoter II
gccggaataactccctataatgcgccacca

SEQ ID NO: 244 - DNA, 30 bp, Artificial Sequence
  Synthetic: BBa_S03331, --Specify Parts List--
ttgacaagcttttcctcagctccgtaaact

SEQ ID NO: 245 - DNA, 30 bp, Artificial Sequence
  Synthetic: BBa_J45992, Full-length stationary phase osmY promoter
ggtttcaaaattgtgatctatatttaacaa

SEQ ID NO: 246 - DNA, 30 bp, Artificial Sequence; identical to SEQ ID NO: 245
  Synthetic: BBa_J45993, Minimal stationary phase osmY promoter

SEQ ID NO: 247 - DNA, 30 bp, Artificial Sequence
  Synthetic: BBa_J45504, htpG Heat Shock Promoter
tctattccaataaagaaatcttcctgcgtg

SEQ ID NO: 248 - DNA, 30 bp, Artificial Sequence
  Synthetic: BBa_K1895002, dnaK Promoter
gaccgaatatatagtggaaacgtttagatg

SEQ ID NO: 249 - DNA, 30 bp, Artificial Sequence
  Synthetic: BBa_K1895003, htpG Promoter
ccacatcctgtttttaaccttaaaatggca

SEQ ID NO: 250 - DNA, 30 bp, Artificial Sequence
  Synthetic: BBa_K143012, Promoter veg a constitutive promoter for B. subtilis
aaaaatgggctcgtgttgtacaataaatgt

SEQ ID NO: 251 - DNA, 30 bp, Artificial Sequence
  Synthetic: BBa_K143013, Promoter 43 a constitutive promoter for B. subtilis
aaaaaaagcgcgcgattatgtaaaatataa

SEQ ID NO: 252 - DNA, 30 bp, Artificial Sequence
  Synthetic: BBa_K780003, Strong constitutive promoter for Bacillus subtilis
aattgcagtaggcatgacaaaatggactca

SEQ ID NO: 253 - DNA, 30 bp, Artificial Sequence
  Synthetic: BBa_K823000, PliaG
caagcttttcctttataatagaatgaatga

SEQ ID NO: 254 - DNA, 30 bp, Artificial Sequence
  Synthetic: BBa_K823002, PlepA
tctaagctagtgtattttgcgtttaatagt

SEQ ID NO: 255 - DNA, 30 bp, Artificial Sequence
  Synthetic: BBa_K823003, Pveg
aatgggctcgtgttgtacaataaatgtagt

SEQ ID NO: 256 - DNA, 30 bp, Artificial Sequence
  Synthetic: BBa_K143010, Promoter ctc for B. subtilis
atccttatcgttatgggtattgtttgtaat

SEQ ID NO: 257 - DNA, 30 bp, Artificial Sequence
  Synthetic: BBa_K143011, Promoter gsiB for B. subtilis
taaaagaattgtgagcgggaatacaacaac

SEQ ID NO: 258 - DNA, 30 bp, Artificial Sequence; identical to SEQ ID NO: 251
  Synthetic: BBa_K143013, Promoter 43 a constitutive promoter for B. subtilis

SEQ ID NO: 259 - DNA, 30 bp, Artificial Sequence
  Synthetic: BBa_K112706, Pspv2 fromSalmonella
tacaaaataattcccctgcaaacattatca

SEQ ID NO: 260 - DNA, 30 bp, Artificial Sequence
  Synthetic: BBa_K112707, Pspv fromSalmonella
tacaaaataattcccctgcaaacattatcg

SEQ ID NO: 261 - DNA, 30 bp, Artificial Sequence
  Synthetic: BBa_I712074, T7 promoter (strong promoter from T7 bacteriophage)
agggaatacaagctacttgttctttttgca

SEQ ID NO: 262 - DNA, 23 bp, Artificial Sequence
  Synthetic: BBa_I719005, T7 Promoter
taatacgactcactatagggaga

SEQ ID NO: 263 - DNA, 28 bp, Artificial Sequence
  Synthetic: BBa_J34814, T7 Promoter
gaatttaatacgactcactatagggaga

SEQ ID NO: 264 - DNA, 19 bp, Artificial Sequence
  Synthetic: BBa_J64997, T7 consensus -10 and rest
taatacgactcactatagg

SEQ ID NO: 265 - DNA, 30 bp, Artificial Sequence
  Synthetic: BBa_K113010, overlapping T7 promoter
gagtcgtattaatacgactcactatagggg

SEQ ID NO: 266 - DNA, 30 bp, Artificial Sequence
  Synthetic: BBa_K113011, more overlapping T7 promoter
agtgagtcgtactacgactcactatagggg

SEQ ID NO: 267 - DNA, 30 bp, Artificial Sequence
  Synthetic: BBa_K113012, weaken overlapping T7 promoter
gagtcgtattaatacgactctctatagggg

SEQ ID NO: 268 - DNA, 18 bp, Artificial Sequence
  Synthetic: BBa_K1614000, T7 promoter for expression of functional RNA
taatacgactcactatag

SEQ ID NO: 269 - DNA, 23 bp, Artificial Sequence
  Synthetic: BBa_R0085, T7 Consensus Promoter Sequence
taatacgactcactatagggaga

SEQ ID NO: 270 - DNA, 23 bp, Artificial Sequence
  Synthetic: BBa_R0180, T7 RNAP promoter
ttatacgactcactatagggaga

SEQ ID NO: 271 - DNA, 23 bp, Artificial Sequence
  Synthetic: BBa_R0181, T7 RNAP promoter
gaatacgactcactatagggaga

SEQ ID NO: 272 - DNA, 23 bp, Artificial Sequence
  Synthetic: BBa_R0182, T7 RNAP promoter
taatacgtctcactatagggaga

SEQ ID NO: 273 - DNA, 23 bp, Artificial Sequence
  Synthetic: BBa_R0183, T7 RNAP promoter
tcatacgactcactatagggaga

SEQ ID NO: 274 - DNA, 30 bp, Artificial Sequence
  Synthetic: BBa_Z0251, T7 strong promoter
taatacgactcactatagggagaccacaac

SEQ ID NO: 275 - DNA, 30 bp, Artificial Sequence
  Synthetic: BBa_Z0252, T7 weak binding and processivity
taattgaactcactaaagggagaccacagc

SEQ ID NO: 276 - DNA, 30 bp, Artificial Sequence
  Synthetic: BBa_Z0253, T7 weak binding promoter
cgaagtaatacgactcactattagggaaga

SEQ ID NO: 277 - DNA, 19 bp, Artificial Sequence
  Synthetic: BBa_J64998, consensus -10 and rest from SP6
atttaggtgacactataga

SEQ ID NO: 278 - DNA, 30 bp, Artificial Sequence
  Synthetic: BBa_I766555, pCyc (Medium) Promoter
acaaacacaaatacacacactaaattaata

SEQ ID NO: 279 - DNA, 30 bp, Artificial Sequence
  Synthetic: BBa_I766556, pAdh (Strong) Promoter
ccaagcatacaatcaactatctcatataca

SEQ ID NO: 280 - DNA, 30 bp, Artificial Sequence
  Synthetic: BBa_I766557, pSte5 (Weak) Promoter
gatacaggatacagcggaaacaacttttaa

SEQ ID NO: 281 - DNA, 30 bp, Artificial Sequence
  Synthetic: BBa_J63005, yeast ADH1 promoter
tttcaagctataccaagcatacaatcaact

SEQ ID NO: 282 - DNA, 30 bp, Artificial Sequence
  Synthetic: BBa_K105027, cyc100 minimal promoter
cctttgcagcataaattactatacttctat

SEQ ID NO: 283 - DNA, 30 bp, Artificial Sequence; identical to SEQ ID NO: 282
  Synthetic: BBa_K105028, cyc70 minimal promoter

SEQ ID NO: 284 - DNA, 30 bp, Artificial Sequence; identical to SEQ ID NO: 282
  Synthetic: BBa_K105029, cyc43 minimal promoter

SEQ ID NO: 285 - DNA, 30 bp, Artificial Sequence; identical to SEQ ID NO: 282
  Synthetic: BBa_K105030, cyc28 minimal promoter

SEQ ID NO: 286 - DNA, 30 bp, Artificial Sequence; identical to SEQ ID NO: 282
  Synthetic: BBa_K105031, cyc16 minimal promoter

SEQ ID NO: 287 - DNA, 30 bp, Artificial Sequence
  Synthetic: BBa_K122000, pPGK1
ttatctactttttacaacaaatataaaaca

SEQ ID NO: 288 - DNA, 30 bp, Artificial Sequence; identical to SEQ ID NO: 278
  Synthetic: BBa_K124000, pCYC Yeast Promoter

SEQ ID NO: 289 - DNA, 30 bp, Artificial Sequence
  Synthetic: BBa_K124002, Yeast GPD (TDH3) Promoter
gtttcgaataaacacacataaacaaacaaa

SEQ ID NO: 290 - DNA, 30 bp, Artificial Sequence; identical to SEQ ID NO: 279
  Synthetic: BBa_K319005, yeast mid-length ADH1 promoter

SEQ ID NO: 291 - DNA, 30 bp, Artificial Sequence
  Synthetic: BBa_M31201, Yeast CLB1 promoter region, G2/M cell cycle specific
accatcaaaggaagctttaatcttctcata

SEQ ID NO: 292 - DNA, 30 bp, Artificial Sequence
  Synthetic: BBa_I712004, CMV promoter
agaacccactgcttactggcttatcgaaat

SEQ ID NO: 293 - DNA, 30 bp, Artificial Sequence
  Synthetic: BBa_K076017, Ubc Promoter
ggccgtttttggcttttttgttagacgaag

SEQ ID NO: 294 - DNA, 66 bp, Artificial Sequence; identical to SEQ ID NO: 22
  Synthetic: Plpp

SEQ ID NO: 295 - DNA, 52 bp, Artificial Sequence
  Synthetic: PapFAB46
aaaaagagtattgacttcgcatctttttgtacctataatagattcattgcta

SEQ ID NO: 296 - DNA, 59 bp, Artificial Sequence
  Synthetic: PJ23101+UP element
ggaaaatttttttaaaaaaaaaactttacagctagctcagtcctaggtattatgctagc

SEQ ID NO: 297 - DNA, 59 bp, Artificial Sequence
  Synthetic: PJ23107+UP element
ggaaaatttttttaaaaaaaaaactttacggctagctcagccctaggtattatgctagc

SEQ ID NO: 298 - DNA, 64 bp, Artificial Sequence; identical to SEQ ID NO: 16
  Synthetic: PSYN23119